(19) United States
(12) Patent Application Publication
(10) Pub. No.: US 2024/0327412 A1
(43) Pub. Date: Oct. 3, 2024

(54) FACTOR XIIA INHIBITORS

(71) Applicant: KALVISTA PHARMACEUTICALS LIMITED, Wiltshire (GB)

(72) Inventors: Rebecca Louise DAVIE, Wiltshire (GB); Hannah Joy EDWARDS, Wiltshire (GB); Christophe FROMONT, Wiltshire (GB); William Jack GREVES, Wiltshire (GB); Sally Louise MARSH, Wiltshire (GB); Carl Leslie NORTH, Wiltshire (GB); Alicja Stela OBARA, Wiltshire (GB); Jake Simon PEAT, Wiltshire (GB); Stephen John PETHEN, Wiltshire (GB); David Philip ROOKER, Wiltshire (GB); David Edward CLARK, Essex (GB)

(21) Appl. No.: 18/546,793

(22) PCT Filed: Feb. 18, 2022
(86) PCT No.: PCT/GB2022/050447

§ 371 Date: Aug. 17, 2023

Related U.S. Application Data
(60) Provisional application No. 63/151,178, filed on Feb. 19, 2021.
(60) Provisional application No. 63/169,607, filed on Apr. 1, 2021.
(60) Provisional application No. 63/182,283, filed on Apr. 30, 2021.

(30) Foreign Application Priority Data
Feb. 19, 2021 (GB) 2102384.1
Apr. 1, 2021 (GB) 2104788.1
Apr. 30, 2021 (GB) 2106284.9

(51) Int. Cl. C07D 487/04; A61K 31/437; A61K 31/4725; A61K 31/4985; A61K 31/4995; A61K 31/5025; A61K 31/506; A61K 31/517; A61K 31/519; C07D 401/12; C07D 401/14; C07D 405/14; C07D 413/14; C07D 471/04; C07D 487/08; C07D 519/00

(52) U.S. Cl. CPC C07D 487/04; A61K 31/437; A61K 31/4725; A61K 31/4985; A61K 31/4995; A61K 31/5025; A61K 31/506; A61K 31/517; A61K 31/519; C07D 401/12; C07D 401/14; C07D 405/14; C07D 413/14; C07D 471/04; C07D 487/08; C07D 519/00

(57) ABSTRACT

The present invention provides compounds of formula (I): compositions comprising such compounds: the use of such compounds in medicine; and methods of treating patients with such compounds: wherein A, W, V, Z, U, X, Y and B are as defined herein.

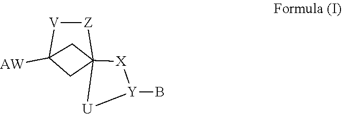

This invention relates to enzyme inhibitors that are inhibitors of Factor XIIa (FXIIa), and to pharmaceutical compositions comprising, and uses of, such inhibitors.

### BACKGROUND TO THE INVENTION

The compounds of the present invention are inhibitors of factor XIIa (FXIIa) and thus have a number of possible therapeutic applications, particularly in the treatment of diseases or conditions in which factor XIIa inhibition is implicated.

FXIIa is a serine protease (EC 3.4.21.38) derived from its zymogen precursor, factor XII (FXII), which is expressed by the F12 gene. Single chain FXII has a low level of amidolytic activity that is increased upon interaction with negatively charged surfaces and has been implicated in its activation (see Invanov et al., Blood. 2017 Mar. 16; 129(11):1527-1537. doi: 10.1182/blood-2016-10-744110). Proteolytic cleavage of FXII to heavy and light chains of FXIIa dramatically increases catalytic activity. FXIIa that retains its full heavy chain is αFXIIa. FXIIa that retains a small fragment of its heavy chain is βFXIIa. The separate catalytic activities of αFXIIa and βFXIIa contribute to the activation and biochemical functions of FXIIa. Mutations and polymorphisms in the F12 gene can alter the cleavage of FXII and FXIIa.

FXIIa has a unique and specific structure that is different from many other serine proteases. For instance, the Tyr99 in FXIIa points towards the active site, partially blocking the S2 pocket and giving it a closed characteristic. Other serine proteases containing a Tyr99 residue (e.g. FXa, tPA and FIXa) have a more open S2 pocket. Moreover, in several trypsin-like serine proteases the P4 pocket is lined by an “aromatic box” which is responsible for the P4-driven activity and selectivity of the corresponding inhibitors. However, FXIIa has an incomplete “aromatic box” resulting in more open P4 pocket. See e.g. “Crystal structures of the recombinant β-factor XIIa protease with bound Thr-Arg and Pro-Arg substrate mimetics” M. Pathak et al., Acta. Cryst. 2019, D75, 1-14; “Structures of human plasma β-factor XIIa cocrystallized with potent inhibitors” A Dementiev et al., Blood Advances 2018, 2(5), 549-558; “Design of Small-Molecule Active-Site Inhibitors of the S1A Family Proteases as Procoagulant and Anticoagulant Drugs” P. M. Fischer, J. Med. Chem., 2018, 61(9), 3799-3822; “Assessment of the protein interaction between coagulation factor XII and corn trypsin inhibitor by molecular docking and biochemical validation” B. K. Hamad et al. Journal of Thrombosis and Haemostasis, 15: 1818-1828.

FXIIa converts plasma prekallikrein (PK) to plasma kallikrein (PKa), which provides positive feedback activation of FXII to FXIIa. FXII, PK, and high molecular weight kininogen (HK) together represent the contact system. FXIIa mediated conversion of plasma prekallikrein to plasma kallikrein can cause subsequent cleavage of HK to generate bradykinin, a potent inflammatory hormone that can also increase vascular permeability, which has been implicated in disorders such as hereditary angioedema (HAE). The contact system is activated via a number of mechanisms, including interactions with negatively charged surfaces, negatively charged molecules, unfolded proteins, artificial surfaces, foreign tissue (e.g. biological transplants, that include bio-prosthetic heart valves, and organ/tissue transplants), bacteria, and biological surfaces (including endothelium and extracellular matrix) that mediate assembly of contact system components. In addition, the contact system is activated by plasmin, and cleavage of FXII by other enzymes can facilitate its activation.

Activation of the contact system leads to activation of the kallikrein kinin system (KKS), complement system, and intrinsic coagulation pathway (see https://www.genome.jp/kegg-bin/show_pathway?map04610). In addition, FXIIa has additional substrates both directly, and indirectly via PKa, including Proteinase-activated receptors (PARs), plasminogen, and neuropeptide Y (NPY) which can contribute to the biological activity of FXIIa. Inhibition of FXIIa could provide clinical benefits by treating diseases and conditions associated with these systems, pathways, receptors, and hormones.

PKa activation of PAR2 mediates neuroinflammation and may contribute to neuroinflammatory disorders including multiple sclerosis (see Göbel et al., Proc Natl Acad Sci USA. 2019 Jan. 2; 116(1):271-276. doi: 10.1073/pnas.1810020116). PKa activation of PAR1 and PAR2 on vascular smooth muscle cells has been implicated in vascular hypertrophy and atherosclerosis (see Abdallah et al., J Biol Chem. 2010 Nov. 5; 285(45):35206-15. doi: 10.1074/jbc.M110.171769). FXIIa activation of plasminogen to plasmin contributes to fibrinolysis (see Konings et al., Thromb Res. 2015 August; 136(2):474-80. doi: 10.1016/j.thromres.2015.06.028). PKa proteolytically cleaves NPY and thereby alters its binding to NPY receptors (Abid et al., J Biol Chem. 2009 Sep. 11; 284(37):24715-24. doi: 10.1074/jbc.M109.035253). Inhibition of FXIIa could provide clinical benefits by treating diseases and conditions caused by PAR signaling, NPY metabolism, and plasminogen activation.

FXIIa-mediated activation of the KKS results in the production of bradykinin (BK), which can mediate, for example, angioedema, pain, inflammation, vascular hyperpermeability, and vasodilatation (see Kaplan et al., Adv Immunol. 2014; 121:41-89. doi: 10.1016/B978-0-12-800100-4.00002-7; and Hopp et al., J Neuroinflammation. 2017 Feb. 20; 14(1):39. doi: 10.1186/s12974-017-0815-8). Garadacimab (CSL-312), a monoclonal antibody inhibitory against FXIIa, recently completed a phase 2 study where monthly prophylactic subcutaneous treatment was reported to be well tolerated and effective in preventing attacks in patients with type I/II hereditary angioedema (HAE), which results in intermittent swelling of face, hands, throat, gastro-intestinal tract and genitals (see https://www.clinicaltrials.gov/ct2/show/NCT03712228 and Craig et al., 1451, Allergy. 2020; 75 (Suppl. 109):5-99. doi: 10.1111/all.14504). Mutations in FXII that facilitate its activation to FXIIa have been identified as a cause of HAE (see Björkqvist et al., J Clin Invest. 2015 Aug. 3; 125(8):3132-46. doi: 10.1172/JCI77139; and de Maat et al., J Allergy Clin Immunol. 2016 November; 138(5):1414-1423.e9. doi: 10.1016/j.jaci.2016.02.021). Since FXIIa mediates the generation of PK to PKa, inhibitors of FXIIa could provide protective effects of all form of BK-mediated angioedema, including HAE and non-hereditary bradykinin-mediated angioedema (BK-AEnH).

“Hereditary angioedema” can be defined as any disorder characterised by recurrent episodes of bradykinin-mediated angioedema (e.g. severe swelling) caused by an inherited genetic dysfunction/fault/mutation. There are currently three known categories of HAE: (i) HAE type 1, (ii) HAE type 2, and (iii) normal C1 inhibitor HAE (normal C1-Inh HAE). However, work on characterizing the etiologies of HAE is ongoing so it is expected that further types of HAE might be defined in the future.

Without wishing to be bound by theory, it is thought that HAE type 1 is caused by mutations in the SERPING1 gene that lead to reduced levels of C1 inhibitor in the blood. Without wishing to be bound by theory, it is thought that HAE type 2 is caused by mutations in the SERPING1 gene that lead to dysfunction of the C1 inhibitor in the blood. Without wishing to be bound by theory, the cause of normal C1-Inh HAE is less well defined and the underlying genetic dysfunction/fault/mutation can sometimes remain unknown. What is known is that the cause of normal C1-Inh HAE is not related to reduced levels or dysfunction of the C1 inhibitor (in contrast to HAE types 1 and 2). Normal C1-Inh HAE can be diagnosed by reviewing the family history and noting that angioedema has been inherited from a previous generation (and thus it is hereditary angioedema). Normal C1-Inh HAE can also be diagnosed by determining that there is a dysfunction/fault/mutation in a gene other than those related to C1 inhibitor. For example, it has been reported that dysfunction/fault/mutation with plasminogen can cause normal C1-Inh HAE (see e.g. Veronez et al., Front Med (Lausanne). 2019 Feb. 21; 6:28. doi: 10.3389/fmed.2019.00028; or Recke et al., Clin Transl Allergy. 2019 Feb. 14; 9:9. doi: 10.1186/s13601-019-0247-x.). It has also been reported that dysfunction/fault/mutation with Factor XII can cause normal C1-Inh HAE (see e.g. Mansi et al. 2014 The Association for the Publication of the Journal of Internal Medicine Journal of Internal Medicine, 2015, 277; 585-593; or Maat et al. J Thromb Haemost. 2019 January; 17(1):183-194. doi: 10.1111/jth.14325).

However, angioedemas are not necessarily inherited. Indeed, another class of angioedema is bradykinin mediated angioedema non-hereditary (BK-AEnH), which is not caused by an inherited genetic dysfunction/fault/mutation. Often the underlying cause of BK-AEnH is unknown and/or undefined. However, the signs and symptoms of BK-AEnH are similar to those of HAE, which, without being bound by theory, is thought to be on account of the shared bradykinin mediated pathway between HAE and BK-AEnH. Specifically, BK-AEnH is characterised by recurrent acute attacks where fluids accumulate outside of the blood vessels, blocking the normal flow of blood or lymphatic fluid and causing rapid swelling of tissues such as in the hands, feet, limbs, face, intestinal tract, airway or genitals.

Specific types of BK-AEnH include: non hereditary angioedema with normal C1 Inhibitor (AE-nC1 Inh), which can be environmental, hormonal, or drug induced; acquired angioedema; anaphylaxis associated angioedema; angiotensin converting enzyme (ACE) inhibitor induced angioedema; dipeptidyl peptidase 4 inhibitor induced angioedema; and tPA induced angioedema (tissue plasminogen activator induced angioedema). However, reasons why these factors and conditions cause angioedema in only a relatively small proportion of individuals are unknown.

Environmental factors that can induce AE-nC1 Inh include air pollution (Kedarisetty et al, Otolaryngol Head Neck Surg. 2019 Apr. 30:194599819846446. doi: 10.1177/0194599819846446) and silver nanoparticles such as those used as antibacterial components in healthcare, biomedical and consumer products (Long et al., Nanotoxicology. 2016; 10(4):501-11. doi: 10.3109/17435390.2015.1088589).

Various publications suggest a link between the bradykinin and contact system pathways and BK-AEnHs, and also the potential efficacy of treatments, see e.g.: Bas et al. (N Engl J Med 2015; Leibfried and Kovary. J Pharm Pract 2017); van den Elzen et al. (Clinic Rev Allerg Immunol 2018); Han et al (JCI 2002).

For instance, BK-medicated AE can be caused by thrombolytic therapy. For example, tPA induced angioedema is discussed in various publications as being a potentially life threatening complication following thrombolytic therapy in acute stroke victims (see e.g. Simão et al., Blood. 2017 Apr. 20; 129(16):2280-2290. doi: 10.1182/blood-2016-09-740670; Fröhlich et al., Stroke. 2019 Jun. 11:STROKEAHA119025260. doi: 10.1161/STROKEAHA.119.025260; Rathbun, Oxf Med Case Reports. 2019 Jan. 24; 2019(1):omyl12. doi: 10.1093/omcr/omyl12; Lekoubou et al., Neurol Res. 2014 July; 36(7):687-94. doi: 10.1179/1743132813Y.0000000302; Hill et al., Neurology. 2003 May 13; 60(9):1525-7).

Stone et al. (Immunol Allergy Clin North Am. 2017 August; 37(3):483-495.) reports that certain drugs can cause angioedema.

Scott et al. (Curr Diabetes Rev. 2018; 14(4):327-333. doi: 10.2174/1573399813666170214113856) reports cases of dipeptidyl Peptidase-4 Inhibitor induced angioedema.

Hermanrud et al., (BMJ Case Rep. 2017 Jan. 10; 2017. pii: bcr2016217802) reports recurrent angioedema associated with pharmacological inhibition of dipeptidyl peptidase IV and also discusses acquired angioedema related to angiotensin-converting enzyme inhibitors (ACEI-AAE). Kim et al. (Basic Clin Pharmacol Toxicol. 2019 January; 124(1):115-122. doi: 10.1111/bcpt.13097) reports angiotensin II receptor blocker (ARB)-related angioedema. Reichman et al., (Pharmacoepidemiol Drug Saf. 2017 October; 26(10):1190-1196. doi: 10.1002/pds.4260) also reports angioedema risk for patients taking ACE inhibitors, ARB inhibitors and beta blockers. Diestro et al. (J Stroke Cerebrovasc Dis. 2019 May; 28(5):e44-e45. doi: 10.1016/j.jstrokecerebrovasdis.2019.01.030) also reports a possible association between certain angioedemas and ARBs.

Giard et al. (Dermatology. 2012; 225(1):62-9. doi: 10.1159/000340029) reports that bradykinin mediated angioedema can be precipitated by estrogen contraception, so called “oestrogen associated angioedema”.

Contact system mediated activation of the KKS has also been implicated in retinal edema and diabetic retinopathy (see Liu et al., Biol Chem. 2013 March; 394(3):319-28. doi: 10.1515/hsz-2012-0316). FXIIa concentrations are increased in the vitreous fluid from patients with advance diabetic retinopathy and in Diabetic Macular Edema (DME) (see Gao et al., Nat Med. 2007 February; 13(2):181-8. Epub 2007 Jan. 28 and Gao et al., J Proteome Res. 2008 June; 7(6):2516-25. doi: 10.1021/pr800112g). FXIIa has been implicated in mediating both vascular endothelial growth factor (VEGF) independent DME (see Kita et al., Diabetes. 2015 October; 64(10):3588-99. doi: 10.2337/db15-0317) and VEGF mediated DME (see Clermont et al., Invest Ophthalmol V is Sci. 2016 May 1; 57(6):2390-9. doi: 10.1167/iovs.15-18272). FXII deficiency is protective against VEGF induced retinal edema in mice (Clermont et al., ARVO talk 2019). Therefore, it has been proposed that FXIIa inhibition will provide therapeutic effects for diabetic retinopathy and retinal edema caused by retinal vascular hyperpermeability, including DME, retinal vein occlusion, age-related macular degeneration (AMD).

As noted above, the contact system can be activated by interaction with bacteria, and therefore FXIIa has been implicated in the treatment of sepsis and bacterial sepsis (see Morrison et al., J Exp Med. 1974 Sep. 1; 140(3):797-811). Therefore, FXIIa inhibitors could provide therapeutic benefits in treating sepsis, bacterial sepsis and disseminated intravascular coagulation (DIC).

FXIIa mediated activation of the KKS and production of BK have been implicated in neurodegenerative diseases including Alzheimer's disease, multiple sclerosis, epilepsy and migraine (see Zamolodchikov et al., Proc Natl Acad Sci USA. 2015 Mar. 31; 112(13):4068-73. doi: 10.1073/pnas.1423764112; Simões et al., J Neurochem. 2019 August; 150(3):296-311. doi: 10.1111/jnc.14793; Göbel et al., Nat Commun. 2016 May 18; 7:11626. doi: 10.1038/ncomms11626; and https://clinicaltrials.gov/ct2/show/NCT03108469). Therefore, FXIIa inhibitors could provide therapeutic benefits in reducing the progression and clinical symptoms of these neurodegenerative diseases.

FXIIa has also been implicated in anaphylaxis (see Bender et al., Front Immunol. 2017 Sep. 15; 8:1115. doi: 10.3389/fimmu.2017.01115; and Sala-Cunill et al., J Allergy Clin Immunol. 2015 April; 135(4):1031-43.e6. doi: 10.1016/j.jaci.2014.07.057). Therefore, FXIIa inhibitors could provide therapeutic benefits in reducing the clinical severity and incidence of anaphylactic reactions.

The role of FXIIa in coagulation was identified over 50 years ago, and has been extensively documented in publications using biochemical, pharmacological, genetic and molecular studies (see Davie et al., Science. 1964 Sep. 18; 145(3638):1310-2). FXIIa mediated activation of factor XI (FXI) triggers the intrinsic coagulation pathway. In addition, FXIIa can increase coagulation in a FXI independent manner (see Radcliffe et al., Blood. 1977 October; 50(4):611-7; and Puy et al., J Thromb Haemost. 2013 July; 11(7):1341-52. doi: 10.1111/jth.12295). Studies on both humans and experimental animal models have demonstrated that FXII deficiency prolongs activated partial prothrombin time (APTT) without adversely affecting hemostasis (see Renne et al., J Exp Med. 2005 Jul. 18; 202(2):271-81; and Simão et al., Front Med (Lausanne). 2017 Jul. 31; 4:121. doi: 10.3389/fmed.2017.00121). Pharmacological inhibition of FXIIa also prolongs APTT without increasing bleeding (see Worm et al., Ann Transl Med. 2015 October; 3(17):247. doi: 10.3978/j.issn.2305-5839.2015.09.07). These data suggest that inhibition of FXIIa could provide therapeutic effects against thrombosis without inhibiting bleeding. Therefore, FXIIa inhibitors could be used to treat a spectrum of prothrombotic conditions including venous thromboembolism (VTE); cancer associated thrombosis; complications caused by mechanical and bioprosthetic heart valves, catheters, extracorporeal membrane oxygenation (ECMO), left ventricular assisted devices (LVAD), dialysis, cardiopulmonary bypass (CPB); sickle cell disease, joint arthroplasty, thrombosis induced by tPA, Paget-Schroetter syndrome and Budd-Chari syndrome. FXIIa inhibitor could be used for the treatment and/or prevention of thrombosis, edema, and inflammation associated with these conditions.

Surfaces of medical devices that come into contact with blood can cause thrombosis. FXIIa inhibitors may also be useful for treating or preventing thromboembolism by lowering the propensity of devices that come into contact with blood to clot blood. Examples of devices that come into contact with blood include vascular grafts, stents, in-dwelling catheters, external catheters, orthopedic prosthesis, cardiac prosthesis, and extracorporeal circulation systems.

Preclinical studies have shown that FXIIa has been shown to contribute to stroke and its complications following both ischemic stroke, and hemorrhagic accidents (see Barbieri et al., J Pharmacol Exp Ther. 2017 March; 360(3):466-475. doi: 10.1124/jpet.116.238493; Krupka et al., PLOS One. 2016 Jan. 27; 11(1):e0146783. doi: 10.1371/journal.pone.0146783; Leung et al., Transl Stroke Res. 2012 September; 3(3):381-9. doi: 10.1007/s12975-012-0186-5; Simão et al., Blood. 2017 Apr. 20; 129(16):2280-2290. doi: 10.1182/blood-2016-09-740670; and Liu et al., Nat Med. 2011 February; 17(2):206-10. doi: 10.1038/nm.2295). Therefore, FXIIa inhibition may improve clinical neurological outcomes in the treatment of patients with stroke.

FXII deficiency has been shown to reduce the formation of atherosclerotic lesions in Apoe^{-/-} mice (Didiasova et al., Cell Signal. 2018 November; 51:257-265. doi: 10.1016/j.cellsig.2018.08.006). Therefore, FXIIa inhibitors could be used in the treatment of atherosclerosis.

FXIIa, either directly, or indirectly via PKa, has been shown to activate the complement system (Ghebrehiwet et al., Immunol Rev. 2016 November; 274(1):281-289. doi: 10.1111/imr.12469). BK increases complement C3 in the retina, and an in vitreous increase in complement C3 is associated with DME (Murugesan et al., Exp Eye Res. 2019 Jul. 24; 186:107744. doi: 10.1016/j.exer.2019.107744). Both FXIIa and PKa activate the complement system (see Irmscher et al., J Innate Immun. 2018; 10(2):94-105. doi: 10.1159/000484257; and Ghebrehiwet et al., J Exp Med. 1981 Mar. 1; 153(3):665-76).

A phase 2 study to assess the safety and efficacy of CSL312, a FXIIa inhibitor, in the treatment of COVID-19 has been assigned clinicaltrials.gov identifier NCT04409509. Shatzel et al. (Res Pract Thromb Haemost, 2020 May 15; 4(4):500-505. doi: 10.1002/rth2.12349) also relates to investigating the contact system's role in COVID-19.

Wygrecka et al. (“Coagulation factor XII regulates inflammatory responses in human lungs”, European Respiratory Journal 2017 50: PA339; DOI: 10.1183/1393003.congress-2017.PA339) relates to the effect of an accumulation of FXII in acute respiratory distress syndrome (ARDS) lungs.

Wong et al. (“CSL312, a Novel Anti-FXII Antibody, Blocks FXII-Induced IL-6 Production from Primary Non-Diseased and Idiopathic Pulmonary Fibrosis Fibroblasts”, American Journal of Respiratory and Critical Care Medicine 2020; 201:A6363) reports that activated FXII may contribute to lung fibrosis (e.g. idiopathic Pulmonary Fibrosis) through direct stimulation of fibroblasts to produce pro-fibrotic cytokine IL-6.

Göbel et al. (The Coagulation Factors Fibrinogen, Thrombin, and Factor XII in Inflammatory Disorders—A Systematic Review, Front. Immunol., 26 Jul. 2018|https://doi.org/10.3389/fimmu.2018.01731) relates to FXII's role in the rheumatoid arthritis (RA).

Scheffel et al. (Cold-induced urticarial autoinflammatory syndrome related to factor XII activation, Nature Communications volume 11, Article number: 179 (2020)) reports that there is a link between contact system activation and cytokine-mediated inflammation, such as cold-induced urticarial autoinflammatory syndrome.

Peyrou et al. (The kallikrein-kinin pathway as a mechanism for auto-control of brown adipose tissue activity, NATURE COMMUNICATIONS, (2020) 11:2132, https://doi.org/10.1038/s41467-020-16009-x), reports a pathway for controlling brown adipose tissue (BAT) thermogenic activity mediated by the kallikrein-kinin system, which may contribute to expanding the range of potential pharmacological candidates in therapeutic strategies against obesity and associated diseases designed to improve energy expenditure and remove excess blood metabolites through activation of BAT. Impaired BAT activity is associated with obesity and insulin resistance.

Compounds that are said to be FXIIa inhibitors have been described by Rao et al. (“Factor XIIa Inhibitors” WO2018/093695), Hicks et al. (“Factor XIIa Inhibitors” WO2018/093716), Breslow et al. (“Aminotriazole immunomodulators for treating autoimmune diseases” WO2017/123518) and Ponda et al. (“Aminacylindazole immunomodulators for treatment of autoimmune diseases” WO2017/205296 and “Pyranopyrazole and pyrazolopyridine immunomodulators for treatment of autoimmune diseases” WO2019/108565). FXII/FXIIa inhibitors are said to have been described by Nolte et al. (“Factor XII inhibitors for the administration with medical procedures comprising contact with artificial surfaces” WO2012/120128).

Compounds that are said to be modulators of FXIIa have been described by Philippou et al. (“Factor XIIa Inhibitors” WO 2019/211585 and WO 2019/186164). Macrocylic peptides that are said to be inhibitors of FXIIa have been described by Wilbs et al. (Nat Commun 11, 3890 (2020). Doi: 10.1038/s41467-020-17648-w).

To date, no FXIIa inhibitors have been approved for medical use, and there are no small molecule FXIIa inhibitors in clinical development. Although certain known compounds are said to be modulators or inhibitors of FXIIa, these compounds can suffer from limitations such as being non-reversible or covalent binders, being poorly selective for FXIIa over other related enzymes, or not having demonstrated pharmacokinetic properties suitable for oral therapy. For example, compounds with acylating reactivity e.g. acylated aminotriazoles, are typically non-reversible covalent binders, and can sometimes also be unstable in water and/or blood plasma due to their inherent reactivity. Poor selectivity for FXIIa over other serine proteases (such as thrombin, FXa, FXIa, KLK1, plasmin, trypsin) increases the risk of off-target effects, which can be made even worse (i.e. there is typically a higher likelihood of poor selectivity and off-target effects) if the inhibitor is a covalent binder. Therefore, there remains a need to develop new FXIIa inhibitors that are not covalent inhibitors and/or are highly selective for FXIIa in order to e.g. mitigate the risks of non-selectivity and cytotoxicity. There is a particular need to develop a small molecule FXIIa inhibitors as an oral therapy.

In view of the above, there also remains a need to develop new FXIIa inhibitors that will have utility to treat a wide range of disorders, in particular angioedema; HAE, including: (i) HAE type 1, (ii) HAE type 2, and (iii) normal C1 inhibitor HAE (normal C1-Inh HAE); BK-AEnH, including AE-nC1 Inh, ACE and tPA induced angioedema; vascular hyperpermeability; stroke including ischemic stroke and haemorrhagic accidents; retinal edema; diabetic retinopathy; impaired visual acuity; DME; retinal vein occlusion; AMD; neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; anaphylaxis; thrombosis; thromboembolism caused by increased propensity of medical devices that come into contact with blood to clot blood; prothrombotic conditions including disseminated intravascular coagulation (DIC), venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; acute respiratory distress syndrome (ARDS); idiopathic pulmonary fibrosis (IPF); rheumatoid arthritis (RA); cold-induced urticarial autoinflammatory syndrome; obesity; and diabetes. In particular, there remains a need to develop new FXIIa inhibitors.

### DESCRIPTION OF THE INVENTION

The present invention relates to a series of inhibitors of Factor XIIa (FXIIa). The compounds of the invention are potentially useful in the treatment of diseases or conditions in which factor XIIa inhibition is implicated. The invention further relates to pharmaceutical compositions of the inhibitors, to the use of the compositions as therapeutic agents, and to methods of treatment using these compositions.

Specifically, the invention provides compounds of formula (I)

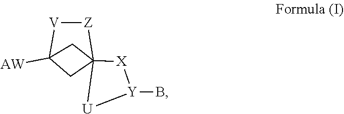

wherein:
U is absent-C(R16)(R17)-, CH₂C(R16)(R17) or C(R16)(R17)CH₂;
-V-Z- is:
   absent, —CH₂—, or —CH₂—O—CH₂; or
V is selected from CH₂, O and NR18, and Z is selected from —C(R16)(R17)-CH₂— and —C(R16)(R17)-; or,
V is selected from —CH₂—C(R16)(R17)- and —C(R16)(R17)-, and Z is selected from CH₂, O and NR18;
   wherein R18 is selected from H, alkyl, (CH₂)₀₋₆-aryl, (CH₂)₀₋₆-heteroaryl^{a}, C(═O)R19, C(═O)OR19, C(═O)NHR19, C(═O)N(alkyl)(R19), C(═O)SR19 and SO₂R19;
   wherein R19 is selected from alkyl, cycloalkyl, (CH₂)₀₋₆-aryl and (CH₂)₀₋₆-heteroaryl^{a};
X is selected from a bond, O, CR1R2, C═O and NR12;
Y is, where possible, selected from O, CR1R2, CR1, C═O, N and NR12;
   R1 is selected from H, alkyl, alkoxy, OH, halo and NR13R14;
   R2 is selected from H and small alkyl;
wherein when one of X or Y is C═O, the other is, where possible, O, CR1R2, CR1, N or NR12;
wherein when X is NR12, Y is, where possible, CR1R2, CR1 or C═O;
wherein when Y is, where possible, NR12 or N, X is a bond, CR1R2 or C═O;
wherein when X is O, Y is, where possible, CR1R2, CR1 or C═O;
wherein when Y, where possible, is O, X is a bond, CR1R2 or C═O;
wherein when X is a bond, Y is, where possible, O, N or NR12;
wherein when U is not absent -V-Z- is absent;
wherein when -V-Z- is not absent, U is absent;
B is selected from:
   (i) heteroaryl^{a};
   (ii) aryl;
   (iii) a 5- to 6-membered non-aromatic heterocyclic ring containing one N ring member, which is unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃; and
   (iv) a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring may be optionally substituted with 1, 2, or 3 substituted by up to three substituents independently selected from alkyl, alkoxy, OH, OCF₃, halo, oxo, CN, and CF₃, wherein the 6,5-bicyclic ring may be attached via the 6- or 5-membered ring;
AW- is selected from:
—(CH₂)₀₋₆—(CHR15)-(CH₂)₀₋₆-A, —(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—NR12-(CH₂)₁₋₆—C(═O)-A, —(CH₂)₀₋₆—NH—C(═O)—(CH₂)₀₋₆-A, —C(═O)NR12-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A and —SO₂—NH-A;
A is a 4- to 15-membered mono-, bi-, or tri-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein when A is a bicyclic ring system, the bicyclic ring system is fused, bridged or spiro;
wherein when A is a tricyclic ring system, each of the three rings in the tricyclic ring system is either fused, bridged or spiro to at least one of the other rings in the tricyclic ring system;
wherein when -V-Z- is —CH₂—, U is absent, and AW- is A-(C═O)—, A may not be substituted by —(CH₂)₀-heteroaryl;
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from (C₁-C6)alkoxy, OH, —NR13R14, —C(═O)OR13, —C(═O)NR13R14, CN, CF₃, halo;
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from (C₁-C₅)alkoxy, OH, CN, CF₃, halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from (C₁-C₆)alkoxy, OH, NR13R14, C(═O)OR13, C(═O)NR13R14, CN, CF₃, halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—O-heteroaryl^{a}, aryl^{b}, —O-aryl^{b}, —(CH₂)₁₋₃-aryl^{b}, —(CH₂)₀₋₃-heteroaryl^{a}, —C(═O)OR13, —C(═O)NR13R14, —(CH₂)₀₋₃—NR13R14, OCF₃ and CF₃;
aryl^{b} is phenyl, biphenyl or naphthyl; aryl^{b} may be optionally substituted with 1, 2 or 3 substituents independently selected from alkyl^{b}, alkoxy, OH, halo, CN, and CF₃;
cycloalkyl is a monocyclic saturated hydrocarbon ring of between 3 and 6 carbon atoms (C₃-C₆);
cycloalkyl may optionally be substituted with 1 or 2 substituents independently selected from alkyl, (C₁-C₆)alkoxy, OH, CN, CF₃, halo;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, and fluoro;
halo is F, Cl, Br, or I;
heteroaryl is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃;
heteroaryl^{a} is a 5, 6, 9 or 10 membered mono- or bi-cyclic aromatic ring, containing, where possible, 1, 2, 3 or 4 ring members independently selected from N, NR12, S and O; heteroaryl^{a} may be optionally substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃;
heteroaryl^{b} is a 5, 6, 9 or 10 membered mono- or bi-cyclic aromatic ring, containing, where possible, 1, 2 or 3 ring members independently selected from N, NR12, S and O; wherein heteroaryl^{b} may be optionally substituted with 1, 2 or 3 substituents independently selected from alkyl^{b}, alkoxy, OH, halo, CN, aryl^{b}, —(CH₂)₁₋₃-aryl^{b}, and CF₃;
R8 is independently selected from H, alkyl, cycloalkyl, and heterocycloalkyl;
heterocycloalkyl is a non-aromatic carbon-containing monocyclic ring containing 3, 4, 5, or 6, ring members, wherein at least one ring member is independently selected from N, NR12, S, and O;
heterocycloalkyl may be optionally be substituted with 1 or 2 substituents independently selected from alkyl, (C₁-C₆)alkoxy, OH, CN, CF₃, halo;
R12 is independently selected from H, alkyl, and cycloalkyl;
R13 and R14 are independently selected from H, alkyl^{b}, aryl^{b} and heteroaryl^{b} or R13 and R14 together with the nitrogen atom to which they are attached form a carbon-containing 4-, 5-, 6- or 7-membered heterocyclic ring, optionally containing an additional heteroatom selected from N, NR12, S, SO, SO₂, and O, which may be saturated or unsaturated with 1 or 2 double bonds and which may be optionally mono- or di-substituted with substituents selected from oxo, alkyl^{b}, alkoxy, OH, halo and CF₃;
R15 is selected from alkyl, halo, CF₃, CN, OH, alkoxy, NR13R14, and CONR13R14;
R16 and R17 are independently selected from H and small alkyl;
and tautomers, isomers, stereoisomers (including enantiomers, diastereoisomers and racemic and scalemic mixtures thereof), deuterated isotopes, and pharmaceutically acceptable salts and/or solvates thereof;
wherein the compound is not N-(2-chlorophenyl)-3-((5-cyano-1H-indazol-1-yl)-methyl)-N-methylbicyclo-[1.1.1]pentane-1-carboxamide.

The compounds of the formula (I) have been developed to be inhibitors of FXIIa, which as noted above, has a unique and specific binding site and there is a need for small molecule FXIIa inhibitors. Compounds of formula (I) can possess characteristics that can be considered suitable for oral delivery e.g. a suitable oral availability profile. The compounds of formula (I) can also avoid including groups associated with covalent binding properties e.g. groups with acylating reactivity such as acylated aminotriazoles, and thus can provide compounds that are reversible inhibitors, to further reduce the risk of off-target effects and cytotoxicity.

The present invention also provides a prodrug of a compound as herein defined, or a pharmaceutically acceptable salt and/or solvate thereof.

The present invention also provides an N-oxide of a compound as herein defined, or a prodrug or pharmaceutically acceptable salt and/or solvate thereof.

It will be understood that “pharmaceutically acceptable salts and/or solvates thereof” means “pharmaceutically acceptable salts thereof”, “pharmaceutically acceptable solvates thereof”, and “pharmaceutically acceptable solvates of salts thereof”.

The compounds of the present invention can be provided as mixtures of more than one stereoisomer. When provided as a mixture of stereoisomers, one stereoisomer can be present at a purity >90% relative to the remaining stereoisomers. More specifically, when provided as a mixture of stereoisomers, one stereoisomer can be present at a purity >95% relative to the remaining stereoisomers.

It will be understood that substituents may be named as its free unbonded structure (e.g. piperidine) or by its bonded structure (e.g. piperidinyl). No difference is intended.

It will be understood that the compounds of the invention comprise several substituents. When any of these substituents is defined more specifically herein, the substituents/optional substituents to these groups described above also apply, unless stated otherwise. For example, B can be heteroaryl^{a}, which more specifically can be isoquinolinyl. In this case, isoquinolinyl can be optionally substituted in the same manner as “heteroaryl^{a}”.

It will be understood that the term “where possible” means that the group, atom, or substituent in question may be present if it is chemically possible to do so, e.g. does not exceed the valencies of chemically stable compounds. For example, Y can, where possible, be N, but only in the instance where

U is not absent. This is because, when U is not absent, the N is already trivalent by virtue of its connection to all of X, U and B, and therefore there is no spare valency for a further substituent (such as an R12 group). Likewise, Y can, where possible, be NR12, but only in the instance where U is absent. This is because, when U is absent, the N of the NR12 is connected to X and B, and therefore has one available valency for the R12 group substituent.

It will be understood that when when U is not absent, -V-Z- is absent, and therefore that pentavalent carbon atoms are not covered by the invention. Similarly, it will be understood that when -V-Z- is not absent, U is absent, and therefore that pentavalent carbon atoms are not covered by the invention. It will be understood that when U is absent, the covalent bonds between U and Y, and between U and the carbon which is attached to X and Z, are also absent. It will be understood from claim 1 that for the carbon which is attached to all of U, X and Z, no configuration of the claim allows for a pentavalent carbon. For example, if -V-Z- is —CH₂—CH₂—, U cannot be CH₂ as this would result in the carbon which is attached to all of U, X and Z having five covalent bonds which is not allowable in any configuration of the claim. Therefore, either U, or -V-Z-, and the covalent bonds which attach them must be absent (or both U, -V-Z-, and the covalent bonds which attach them can be absent).

It will be understood that “X is a bond” means that X does not contain an atom, and provides a covalent bond directly from Y to the carbon which is attached to all of U, X and Z. For example, when X is a bond (i.e. Y is connected to the adjacent carbon by X as a covalent bond), the compound of formula (I) is

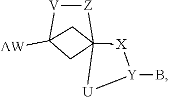

(with A, W, V, Z, U, Y and B as defined in claim 1).

It will be understood that a fused ring system refers to a ring system where two rings in the ring system share two adjacent atoms (i.e one common covalent bond). For example,

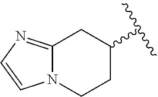

is a fused ring system (specifically a fused bicyclic ring system) which can be considered as an imidazole ring and a piperidine ring sharing a common bond.

It will be understood that a bridged ring system refers to a ring system having two rings sharing three or more atoms. For example,

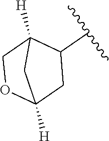 [SMILES: *C1C[C@@H]2C[C@H]1CO2]

is a bridged ring system (specifically a bridged bicyclic ring system) which can be considered as a tetrahydrofuran ring and a pyrrolidine ring joined at a bridge and sharing three common atoms.

It will be understood that a spiro ring system refers to a ring system where two rings in the ring system share one common atom. For example,

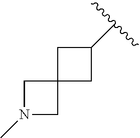 [SMILES: *C1CC2(C1)CN(C)C2]

is a spiro ring system (specifically a spiro bicyclic ring system) which can be considered as a cyclobutane ring and an azetidine ring sharing a common carbon atom.

It will be understood that the ring system A, as defined in formula (I), can be fully saturated, or have any degree of unsaturation. For example, the ring system can be fully saturated, partially unsaturated, aromatic, non-aromatic, or have an aromatic ring bridged, fused or spiro to a non-aromatic ring.

It will be understood that ring system A can contain non-carbon ring members, and that these non-carbon ring members can, where possible, be optionally substituted themselves (as well, or as opposed to the carbon ring members), with the optional substituents included in the definition of A.

It will be understood that when any variable (e.g. alkyl) occurs more than once, its definition on each occurrence is independent of every other occurrence.

It will be understood that combinations of substituents and variables are permissible only if such combinations result in stable compounds.

As used herein the term “bradykinin-mediated angioedema” means hereditary angioedema, and any non-hereditary bradykinin-mediated angioedema. For example, “bradykinin-mediated angioedema” encompasses hereditary angioedema and acute bradykinin-mediated angioedema of unknown origin.

As used herein, the term “hereditary angioedema” means any bradykinin-mediated angioedema caused by an inherited genetic dysfunction, fault, or mutation. As a result, the term “HAE” includes at least HAE type 1, HAE type 2, and normal C1 inhibitor HAE (normal C1-Inh HAE).

More specifically, the invention provides compounds of formula (I), or a tautomer, isomer, stereoisomer (including an enantiomer, a diastereoisomer and a racemic and scalemic mixture thereof), a deuterated isotope, and a pharmaceutically acceptable salt and/or solvate thereof, wherein R18 is selected from alkyl, (CH₂)₀₋₆-aryl, (CH₂)₀₋₆-heteroaryl^{a}, C(═O)R19, C(═O)OR19, C(═O)NHR19, C(═O)N(alkyl)(R19), C(═O)SR19 and SO₂R19.

More specifically, the invention also provides compounds of formula (I) wherein U is absent, which are compounds of formula (Ia)

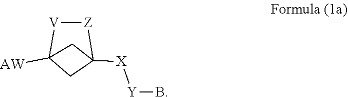

The invention also provides compounds of formula (I) wherein -V-Z- is absent, which are compounds of formula (Ib)

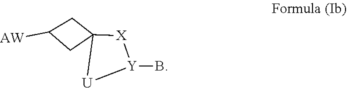

Specifically, the invention provides compounds of formula (I) wherein when -V-Z- is absent and U is absent, and AW-and-XYB are trans to one another which are compounds of formula (1c)

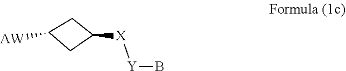

Preferably, when not absent, -V-Z- is selected from:
CH₂—, or
V is selected from CH₂, O and NR18, and Z is selected from —C(R16)(R17)-CH₂— and —C(R16)(R17)-; or,
V is selected from —CH₂—C(R16)(R17)- and —C(R16)(R17)-, and Z is selected from CH₂, O and NR18; or
wherein when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂—, —CH₂—N(R18)- and —N(R18)-CH₂—; or
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂—; or
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—,
—C(CH₃)₂—O—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂—, —CH₂—N(R18)- and —N(R18)-CH₂—; wherein R18 is selected from:
alkyl, (CH₂)₀₋₆-aryl, (CH₂)₀₋₆-heteroaryl^{a}, C(═O)R19, C(═O)OR19, C(═O)NHR19, C(═O)N(alkyl)(R19), C(═O)SR19 and SO₂R19; or
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂—; wherein R18 is selected from:
alkyl, (CH₂)₀₋₆-aryl, (CH₂)₀₋₆-heteroaryl^{a}, C(═O)R19, C(═O)OR19, C(═O)NHR19, C(═O)N(alkyl)(R19), C(═O)SR19 and SO₂R19; or
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂—, —CH₂—N(R18)- and —N(R18)-CH₂— wherein R18 is selected from:
   (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
   wherein R19 is selected from alkyl, (CH₂)₀₋₆-aryl and (CH₂)₀₋₆-heteroaryl^{a}; or
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂— wherein R18 is selected from:
   (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
   wherein R19 is selected from alkyl, (CH₂)₀₋₆-aryl and (CH₂)₀₋₆-heteroaryl^{a}; or
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂,
—CH₂—N(COCH₃), —CH₂—CH₂—, —O—CH₂—CH₂, —CH₂—O—CH₂—CH₂—CH₂—O—, —CH₂—CH₂—CH₂—, —CH₂—N(R18)- and —N(R18)-CH₂—, wherein R18 is selected from:

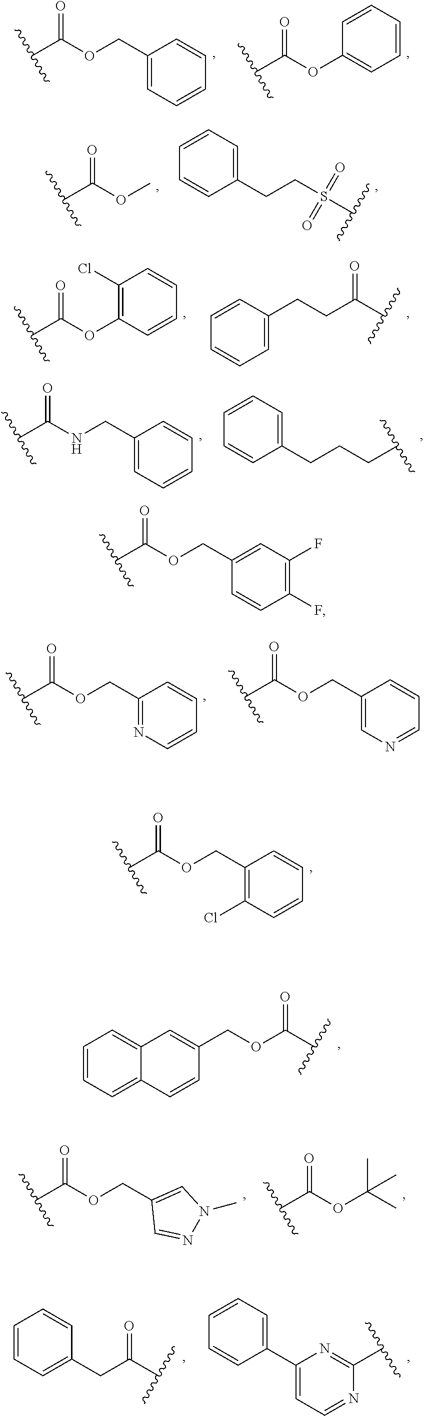

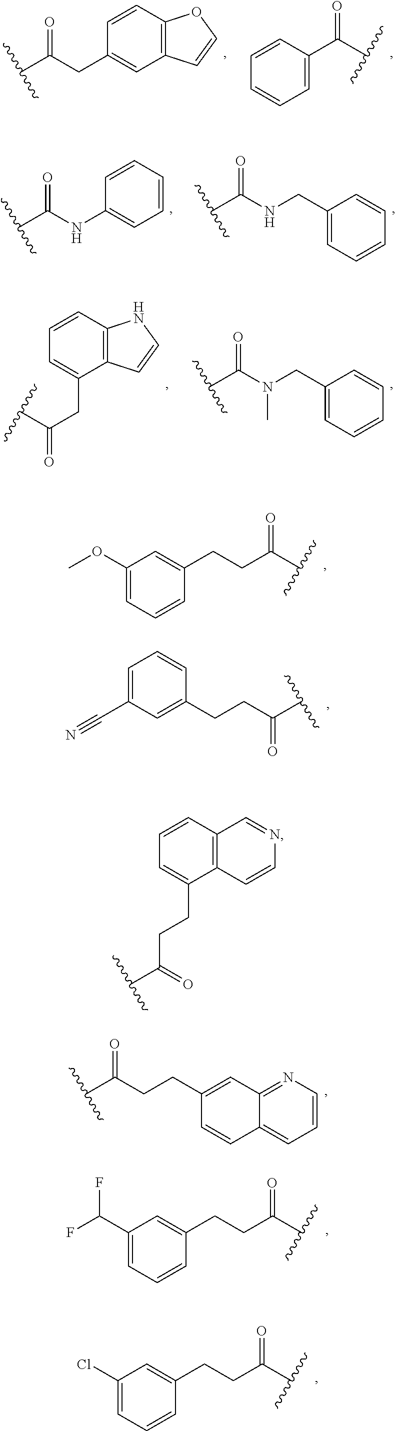

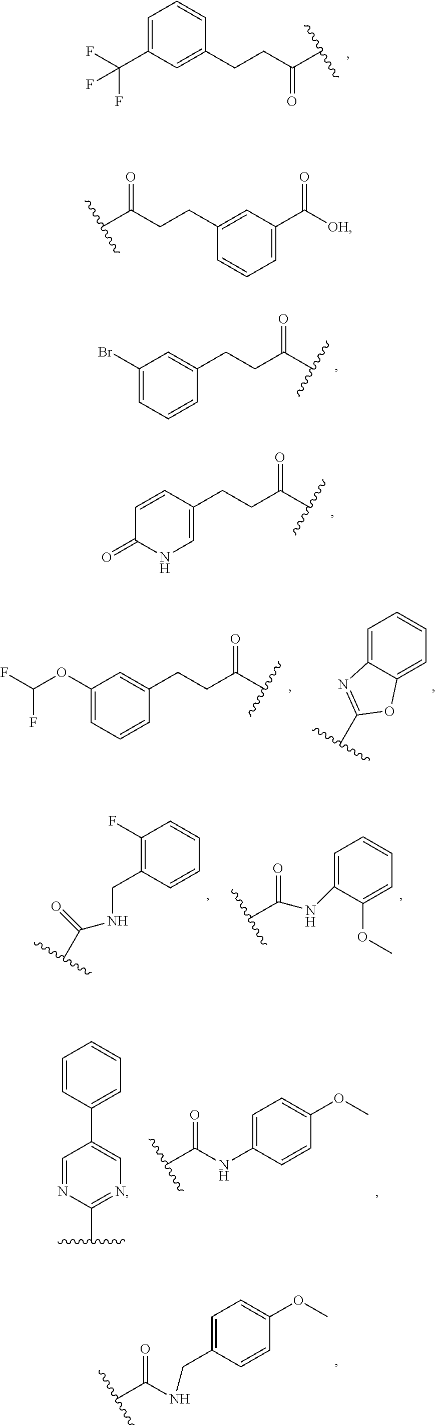

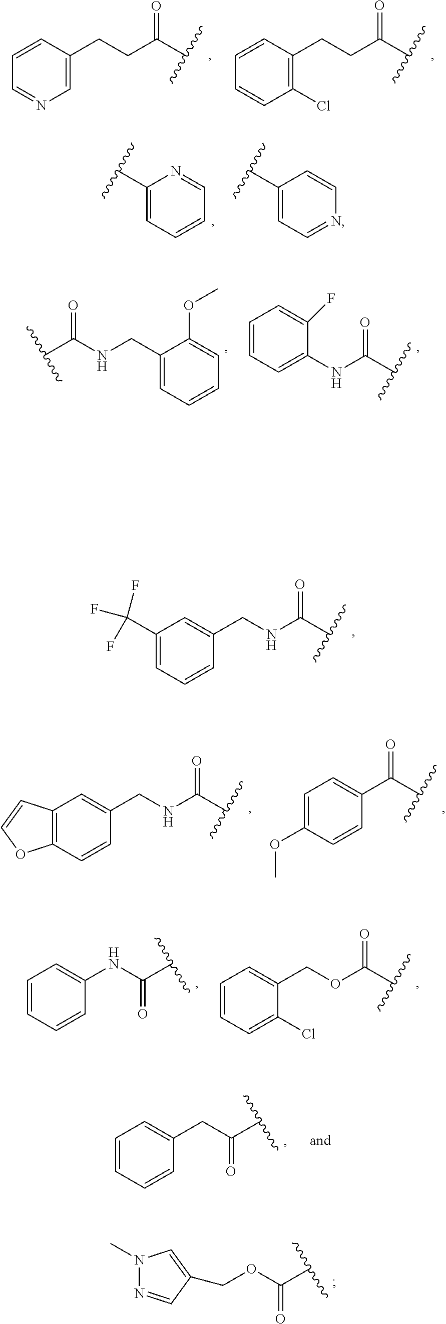

or
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂,
—CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂—, wherein R18 is selected from:

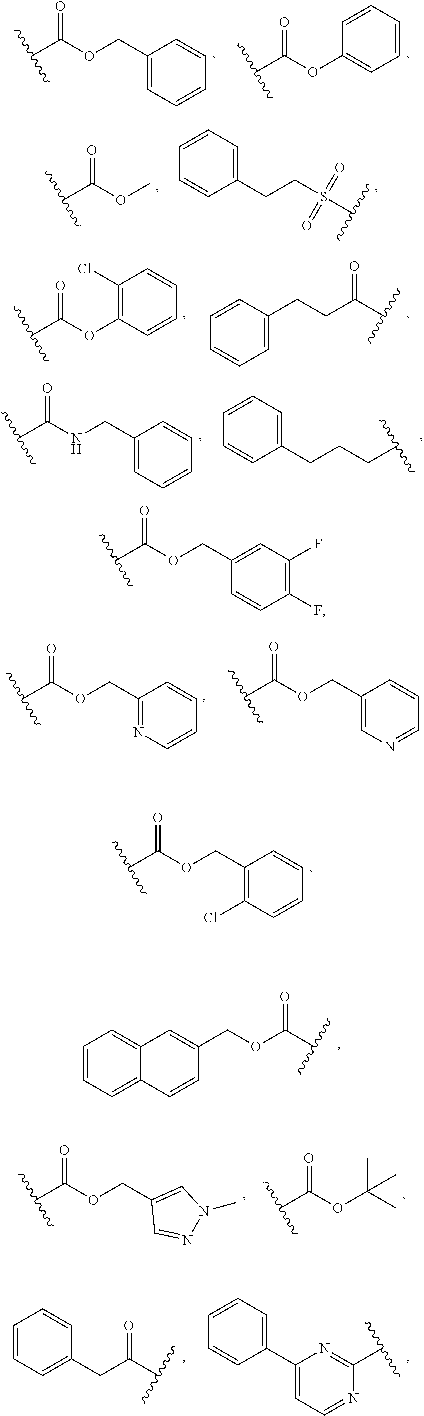

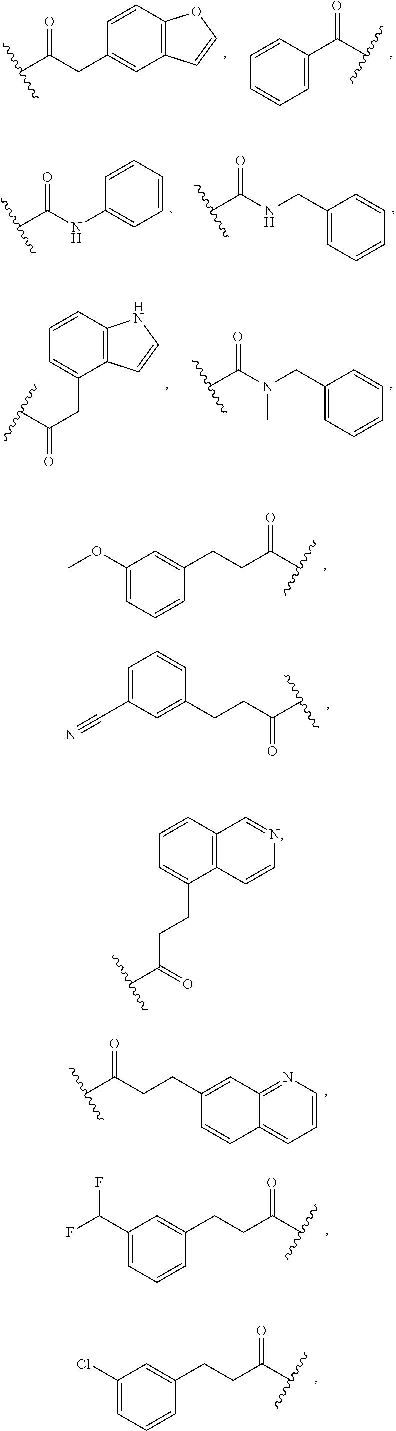

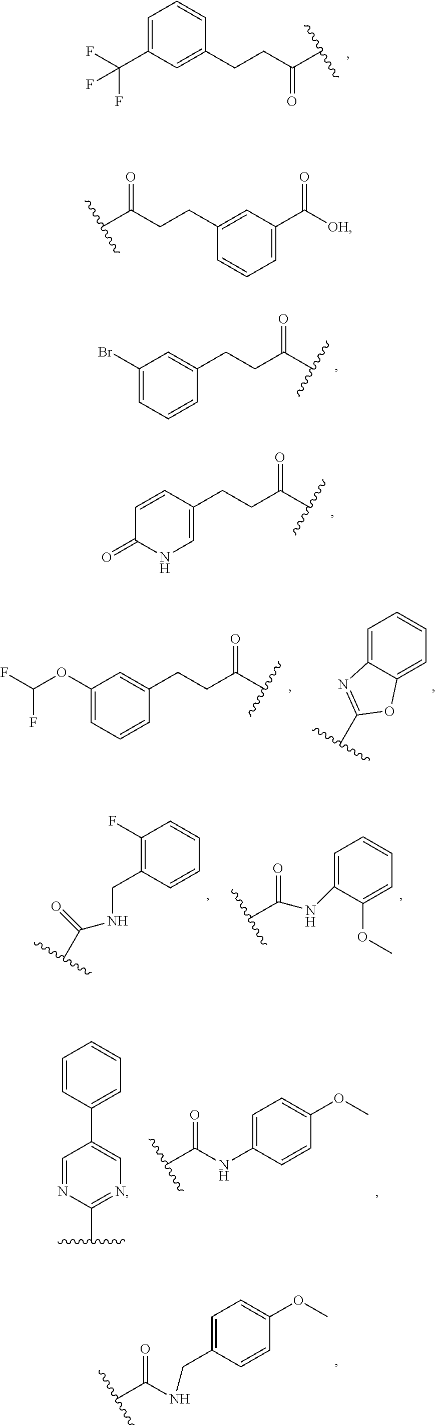

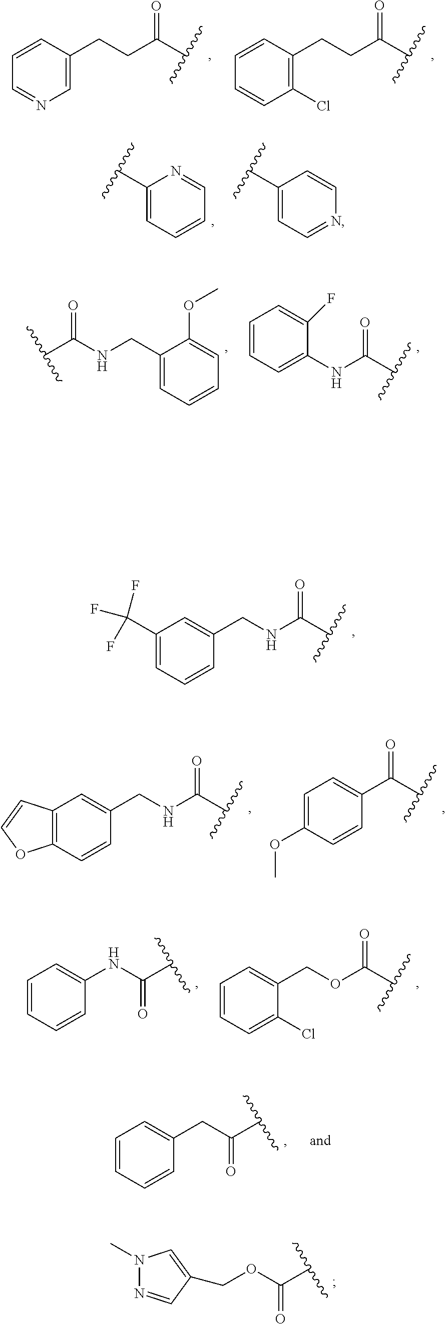

or
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂,
—CH₂—N(COCH₃), —CH₂—CH₂—, —O—CH₂—CH₂, —CH₂—CH₂—O—, and —CH₂—CH₂—CH₂—; or
when -V-Z- is absent:
U is absent, CH₂ or —CH₂CH₂—.

More preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂,
—CH₂—N(COCH₃), —CH₂—CH₂—, —CH₂—CH₂—O—, —CH₂—CH₂—CH₂—, —CH₂—N(R18)- and —N(R18)-CH₂— wherein R18 is selected from:
   (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
   wherein R19 is selected from alkyl, (CH₂)₀₋₆-aryl and (CH₂)₀₋₆-heteroaryl^{a}; or
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —N(COCH₃)—CH₂,
—CH₂—N(COCH₃), —CH₂—CH₂—, —CH₂—CH₂—O—, —CH₂—CH₂—CH₂—, —CH₂—N(R18)- and —N(R18)-CH₂— wherein R18 is selected from:
   (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
   wherein R19 is selected from alkyl, (CH₂)₀₋₆-aryl and (CH₂)₀₋₆-heteroaryl^{a}; or
when -V-Z- is absent:
U is absent, CH₂ or —CH₂CH₂—.

More preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂,
—CH₂—N(COCH₃), —CH₂—CH₂—, —CH₂—CH₂—O—, —CH₂—CH₂—CH₂—, —CH₂—N(R18)- and —N(R18)-CH₂—, wherein R18 is selected from:

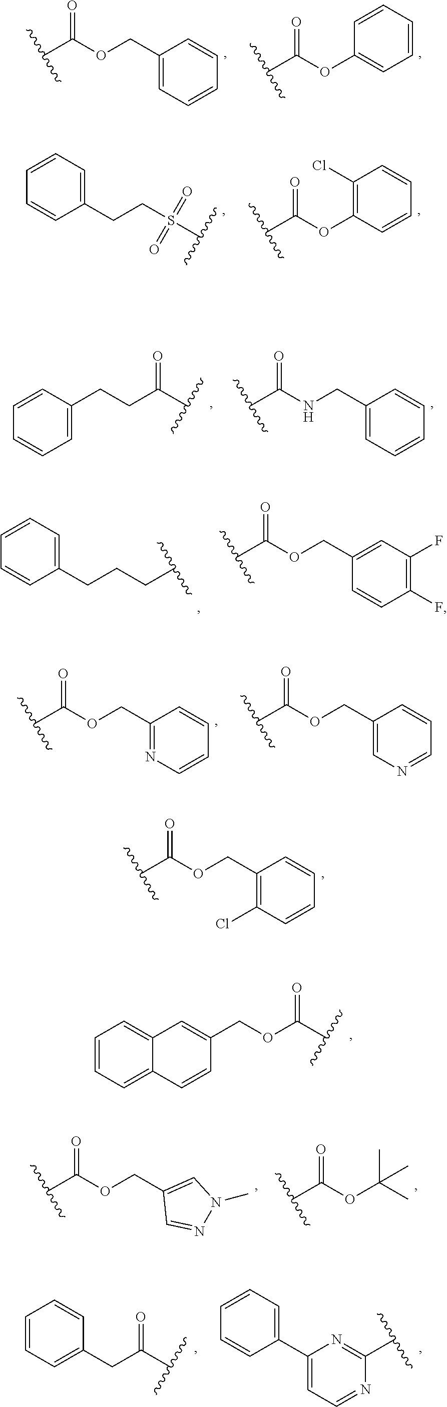

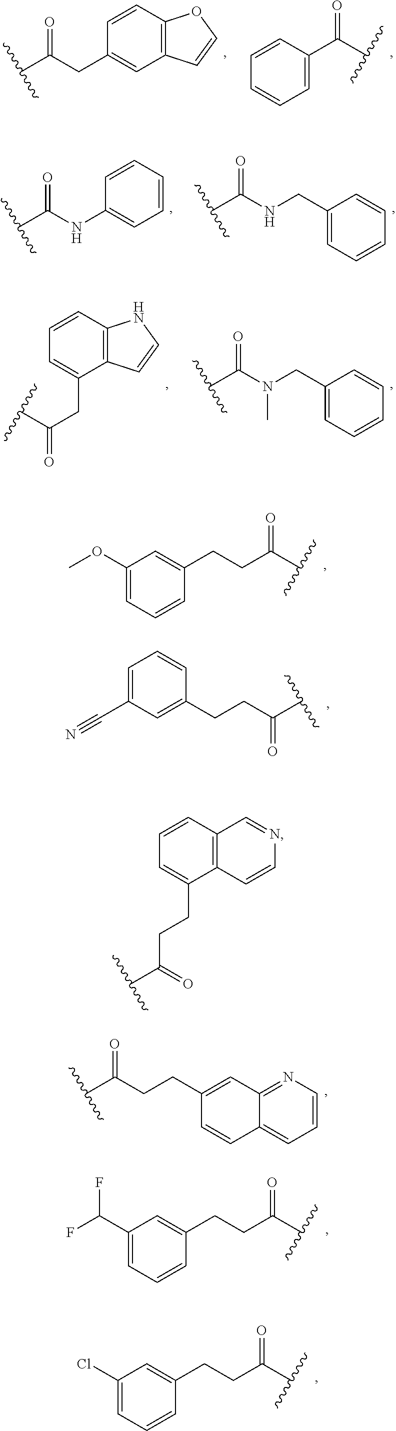

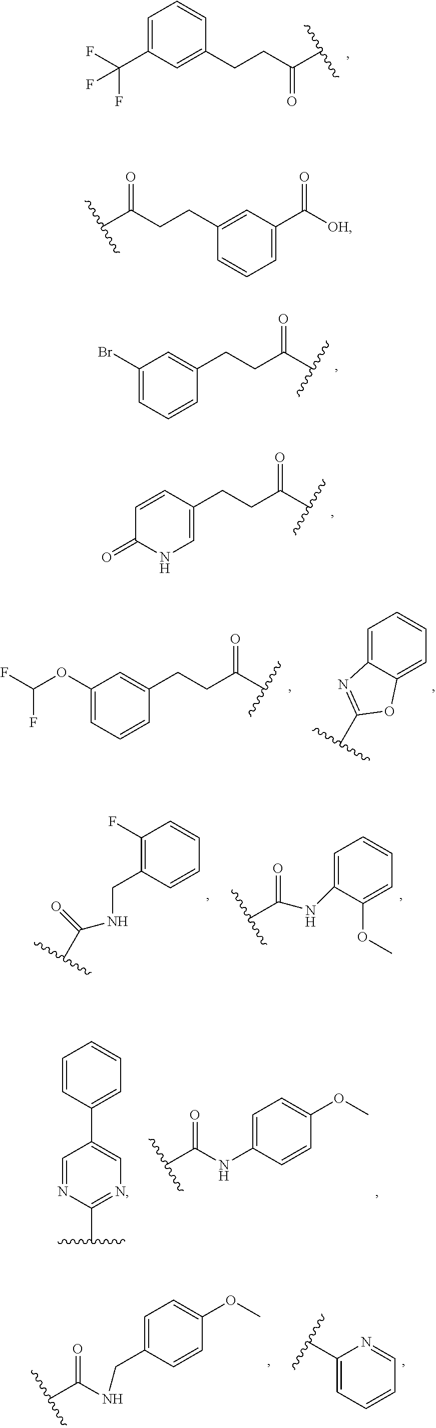

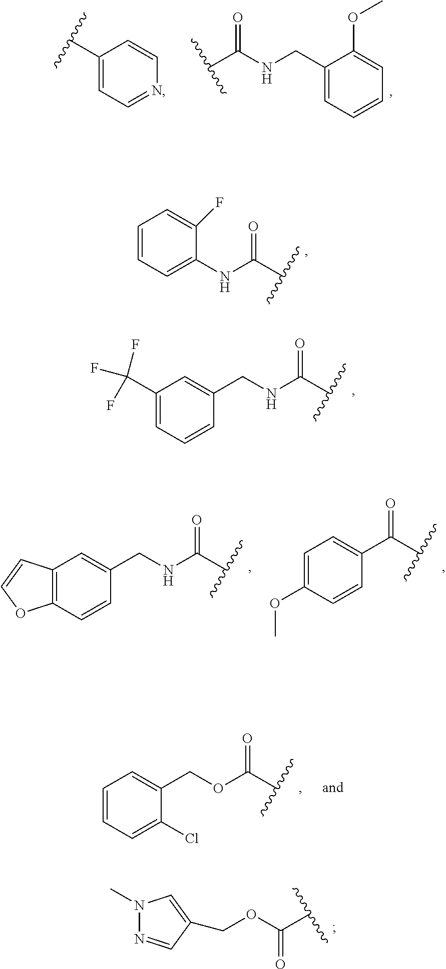

or,
when -V-Z- is absent:
U is absent, CH₂ or —CH₂CH₂—.

Preferably, when not absent, -V-Z- is selected from:
—CH₂—, —O—CH₂—, —O—C(CH₃)₂—, —CH₂—O—, —C(CH₃)₂—O—, —N(COCH₃)—CH₂,
—CH₂—N(COCH₃), —CH₂—CH₂—, —CH₂—CH₂—O—, —CH₂—CH₂—CH₂—, —CH₂—N(R18)- and —N(R18)-CH₂— wherein R18 is selected from:

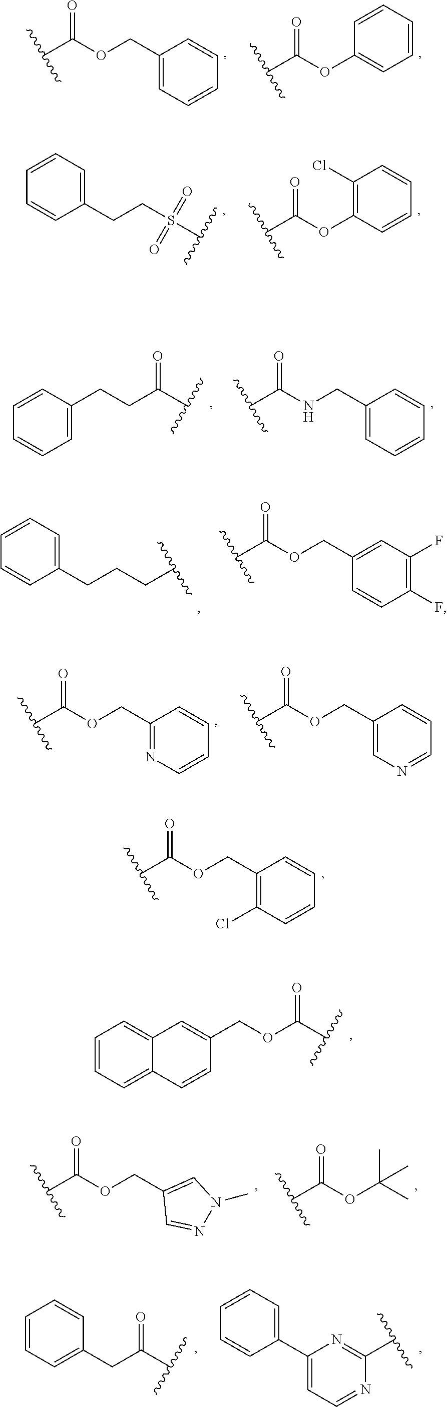

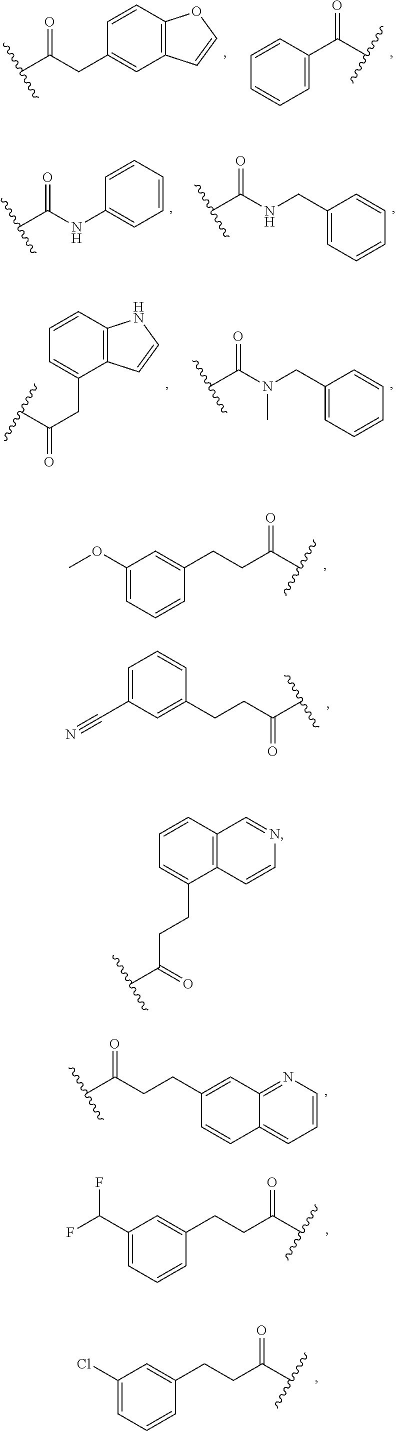

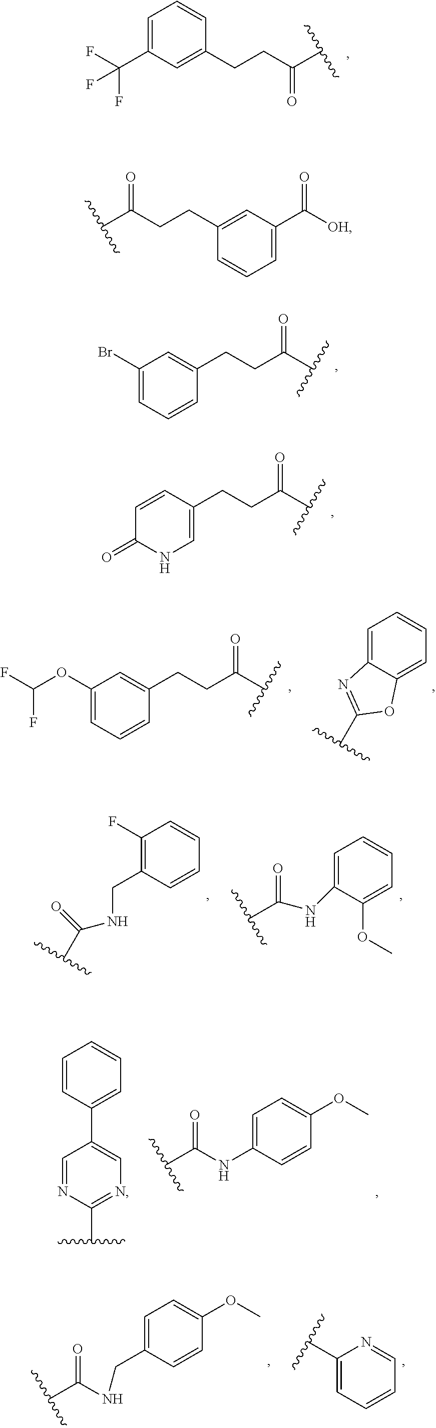

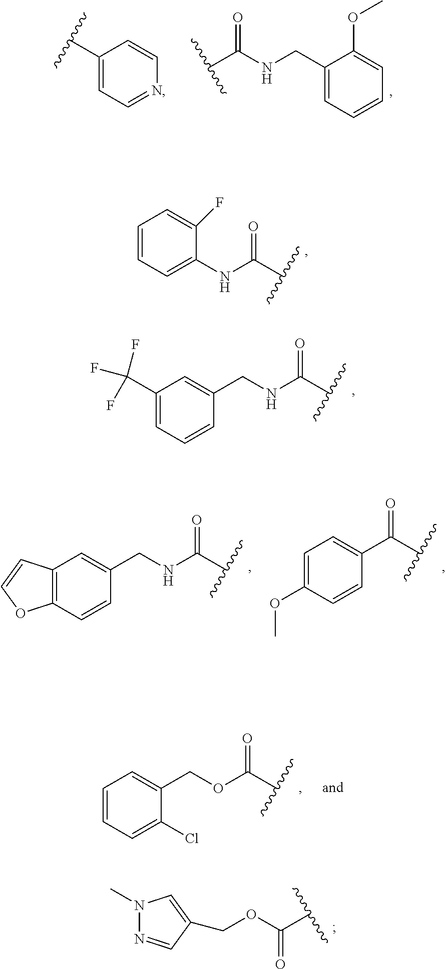

or
wherein when -V-Z- is absent:
U is absent, CH₂ or —CH₂CH₂—.

More preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂,
—CH₂—N(COCH₃), —CH₂—CH₂—, —CH₂—CH₂—O—, and —CH₂—CH₂—CH₂—; or,
when -V-Z- is absent:
U is absent, CH₂ or —CH₂CH₂—.

Yet more preferably, -V-Z- is selected from —CH₂—, —O—CH₂—, —CH₂—O—, —CH₂—CH₂—O—, —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or
when -V-Z- is absent:
U is absent, CH₂ or —CH₂CH₂—.

Yet more preferably, -V-Z- is selected from —CH₂—, —O—CH₂—, —CH₂—O—, —CH₂—CH₂—O—, —CH₂—N(R18)- and —N(R18)-CH₂— wherein R18 is selected from:

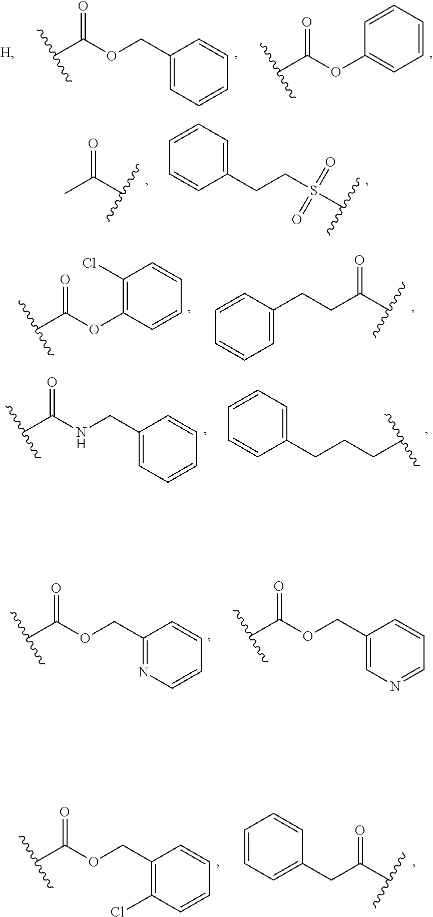

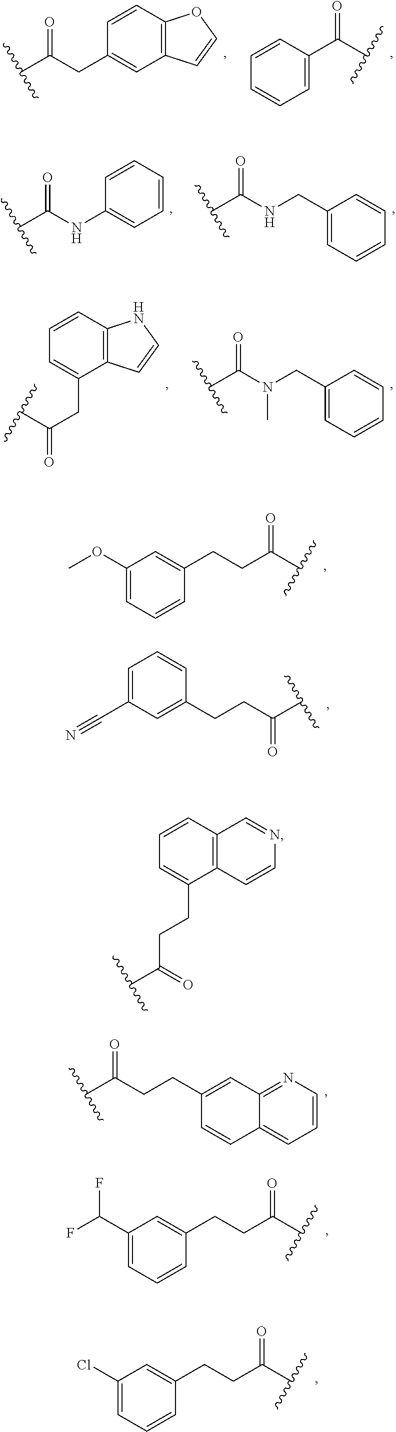

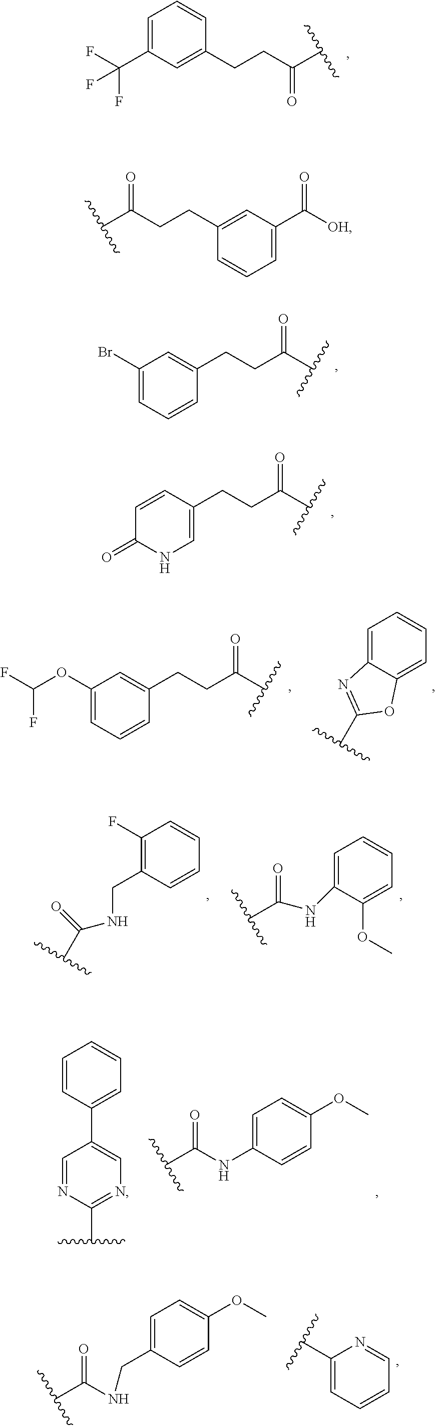

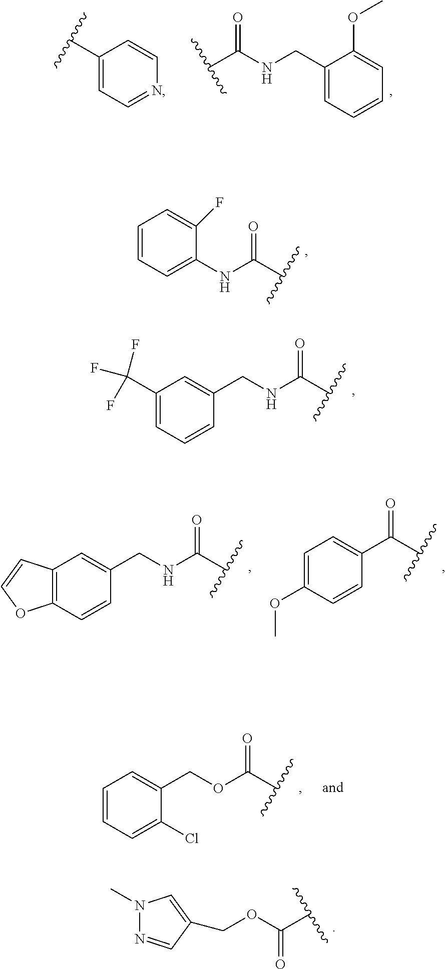

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a}; or
when -V-Z- is absent:
U is absent, CH₂ or —CH₂CH₂—.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

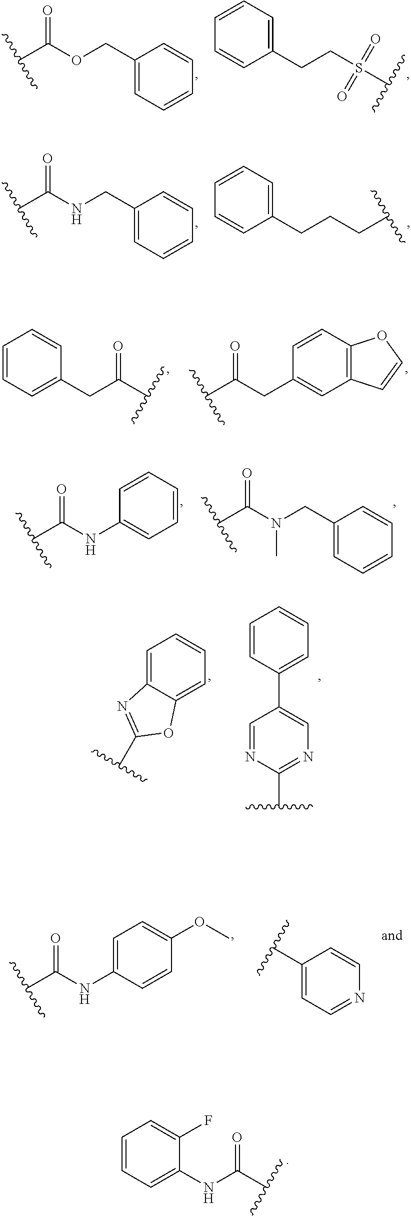

Yet more preferably, -V-Z- is selected from —CH₂—, —O—CH₂—, —CH₂—O— and —CH₂—CH₂—O—.

More preferably, -V-Z- is selected from —O—CH₂— and —CH₂—O—.

More specifically, the invention provides compounds of formula (I) wherein U is absent;

X and Y are independently selected from O, CR1R2, C═O and NR12;
wherein when one of X or Y is C═O, the other is O, CR1R2 or NR12;
wherein when one of X or Y is NR12, the other is CR1R2 or C═O;
wherein when one of X or Y is O, the other is CR1R2 or C═O;
-V-Z- is —CH₂— or;
V is O and Z is CR16R17;
which are compounds of formula (1d)

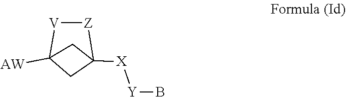

Yet more specifically, the invention provides compounds of formula (I) wherein U is absent and -V-Z- is —CH₂—, which are compounds of formula (1e)

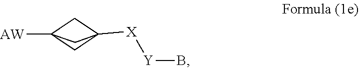

and tautomers, isomers, stereoisomers (including enantiomers, diastereoisomers and racemic and scalemic mixtures thereof), deuterated isotopes, and pharmaceutically acceptable salts and/or solvates thereof.

Alternatively, the invention provides compounds of formula (I) wherein U is absent, V is O and Z is CR16R17 which are compounds of formula (If)

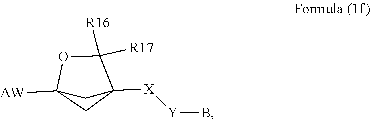

and tautomers, isomers, stereoisomers (including enantiomers, diastereoisomers and racemic and scalemic mixtures thereof), deuterated isotopes, and pharmaceutically acceptable salts and/or solvates thereof.

Preferably, R16 and R17 are both H, or R 16 and R17 are both-CH₃. More preferably, R16 and R17 are both H.

X can be as defined above. In particular, X can be selected from a bond and CR1R2. Preferably, X can be selected from a bond and CH₂. Preferably X is CR1R2. More preferably, X is CH₂.

Y can be as defined above. In particular, Y can be, where possible, selected from O, CR1R2, N and NR12. Preferably Y is, where possible, selected from O, CH₂, N and NH.

Preferably, -V-Z- is —CH₂—, X is CH₂ and Y is NH; -V-Z- is —O—CH₂—, X is CH₂ and Y is NH; -V-Z- is —CH₂—O—, X is CH₂ and Y is NH; -V-Z- is —CH₂—CH₂—O—, X is CH₂ and Y is NH; or -V-Z- is —CH₂—N(R18)-, X is CH₂ and Y is NH; or V-Z- is —N(R18)-CH₂—, X is CH₂ and Y is NH; wherein R18 is selected from:

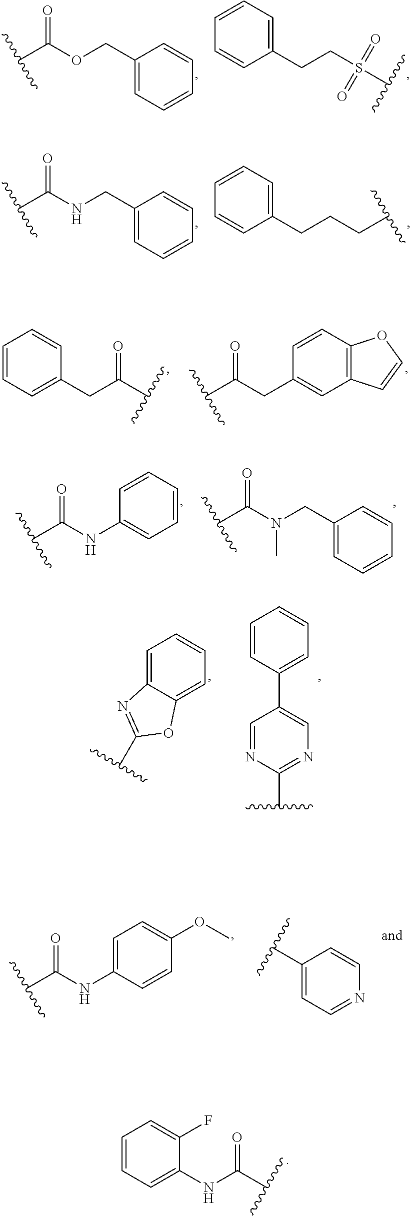

Preferably, -V-Z- is —CH₂—, X is CH₂ and Y is NH; -V-Z- is —O—CH₂—, X is CH₂ and Y is NH; -V-Z- is —CH₂—O—, X is CH₂ and Y is NH; or -V-Z- is —CH₂—CH₂—O—, X is CH₂ and Y is NH.

As noted above, X is selected from a bond, O, CR1R2, C═O and NR12. Y can be, where possible, selected from O, CR1R2, CR1, C═O, N and NR12. When one of X or Y is C═O, the other is, where possible, O, CR1R2, CR1, N or NR12. When X is NR12, Y is, where possible, CR1R2, CR1 or C═O. When Y is, where possible, NR12 or N, X is a bond, CR1R2 or C═O. When X is O, Y is, where possible, CR1R2, CR1 or C═O. When Y, where possible, is O, X is a bond, CR1R2 or C═O. When X is a bond, Y is, where possible, O, N or NR12.

X can be CR1R2. Y can be CR1R2. X and Y can be CR1R2.

R1 can be H. R1 can be alkyl, for example small alkyl such as methyl or ethyl, which can be optionally substituted as for alkyl. R1 can be alkoxy, for example methoxy or ethoxy, which can be optionally substituted as for alkoxy. R1 can be OH. R1 can be halo, for example chloro. R1 can be NR13R14, for example NH₂.

R2 can be H. R2 can be alkyl, for example small alkyl such as methyl or ethyl, which can be optionally substituted as for alkyl. R2 can be alkoxy, for example methoxy or ethoxy, which can be optionally substituted as for alkyl. R2 can be OH. R2 can be halo, for example chloro. R2 can be NR13R14, for example NH₂.

R1 can be H and R2 can be alkyl, for example small alkyl such as methyl or ethyl, which can be optionally substituted as for alkyl. R2 can be alkoxy, for example methoxy or ethoxy, which can be optionally substituted as for alkyl. R2 can be OH. R2 can be halo, for example chloro. R2 can be NR13R14, for example NH₂.

At least one of R1 and R2 can be other than H. At least one of R1 and R2 can be H. Preferably, both R1 and R2 are H.

X can be NR12. Y can be NR12 or N. When X is NR12, Y is, where possible, CR1R2, CR1 or C═O. When Y is, where possible, NR12 or N, X is a bond, CR1R2 or C═O. X can be NR12 and Y can, where possible, be CR1R2 (as defined above). Alternatively, X can be CR1R2 (as defined above) and Y can, where possible, be NR12 or N. X can be NR12 and Y can, where possible, be C═O. Alternatively, Y can, where possible, be NR12 or N and X can be C═O.

R12 can be alkyl, for example small alkyl such as methyl or ethyl, which can be optionally substituted as for alkyl. R12 can be cycloalkyl, for example cyclopropyl, which can be substituted as for cycloalkyl. Preferably, R12 is H.

X can be C═O. Y can, where possible, be C═O. When one of X or Y is C═O, the other is, where possible, O, CR1R2, CR1, N or NR12. X can be C═O and Y can, where possible, be CR1R2 (as defined above). Alternatively, X can be CR1R2 (as defined above) and Y can, where possible, be C═O.

X can be O. Y can, where possible, be O. When X is O, Y is, where possible, CR1R2, CR1 or C═O. When Y, where possible, is O, X is a bond, CR1R2 or C═O. X can be O and Y can, where possible, be CR1R2 (as defined above). Alternatively, X can be CR1R2 (as defined above) and Y can, where possible, be O. X can be O and Y can, where possible, be C═O. Alternatively, X can be C═O and Y can, where possible, be O. X can be O and Y can, where possible, be CR1. X can be a bond and Y can, where possible, be O.

X can be a bond. When X is a bond, Y is, where possible, O, N or NR12. When X is a bond, Y can be, where possible, O. When X is a bond, Y can be, where possible, N. When X is a bond, Y can be, where possible, NR12. Preferably, when X is a bond, Y is, where possible, N or NR12. More preferably, when X is a bond, Y is, where possible, N or NH.

Preferably X is CH₂, and Y is, where possible, N or NR12.

Preferably X is CH₂, and Y is, where possible, N or NH.

Preferably X is CH₂, Y is NR12, and U is absent.

Preferably X is CH₂ and Y is NH.

Preferably X is CH₂, Y is NH and U is absent.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂—, —CH₂—N(R18)- and —N(R18)-CH₂—; or, when -V-Z- is absent: U is absent, CH₂ or —CH₂CH₂—; X is CH₂; and Y is NH or N.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂—; or, when -V-Z- is absent: U is absent, CH₂ or —CH₂CH₂—; X is CH₂; and Y is NH or N.

Preferably, when not absent, -V-Z- is selected from: —CH₂—, —O—CH₂—, —O—C(CH₃)₂—, —CH₂—O—, —C(CH₃)₂—O—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂—, —CH₂—N(R18)- and —N(R18)-CH₂—; wherein R18 is selected from: alkyl, (CH₂)₀₋₆-aryl, (CH₂)₀₋₆-heteroaryl^{a}, C(═O)R19, C(═O)OR19, C(═O)NHR19, C(═O)N(alkyl)(R19), C(═O)SR19 and SO₂R19; or,
when -V-Z- is absent: U is absent, CH₂ or —CH₂CH₂—; X is CH₂; and Y is NH or N.

Preferably, when not absent, -V-Z- is selected from: —CH₂—, —O—CH₂—, —O—C(CH₃)₂—, —CH₂—O—, —C(CH₃)₂—O—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂—; wherein R18 is selected from:
alkyl, (CH₂)₀₋₆-aryl, (CH₂)₀₋₆-heteroaryl^{a}, C(═O)R19, C(═O)OR19, C(═O)NHR19, C(═O)N(alkyl)(R19), C(═O)SR19 and SO₂R19; or,
when -V-Z- is absent: U is absent, CH₂ or —CH₂CH₂—; X is CH₂; and Y is NH or N.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or,
when -V-Z- is absent: U is absent, CH₂ or —CH₂CH₂—; X is CH₂; and Y is NH or N.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CH₂; and Y is NH.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, and —CH₂—CH₂—CH₂—; or,
when -V-Z- is absent: U is absent, CH₂ or —CH₂CH₂—; X is CH₂; and Y is NH.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, and —CH₂—CH₂—CH₂—; or,
when -V-Z- is absent: U is absent, CH₂ or —CH₂CH₂—; X is CH₂; and Y is NH or N.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂—, —CH₂—N(R18)- and —N(R18)-CH₂— wherein R18 is selected from:
   (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
   wherein R19 is selected from alkyl, (CH₂)₀₋₆-aryl and (CH₂)₀₋₆-heteroaryl^{a}; or,
when -V-Z- is absent: U is absent, CH₂ or —CH₂CH₂—; X is CH₂; and Y is NH or N.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂— wherein R18 is selected from:
   (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
   wherein R19 is selected from alkyl, (CH₂)₀₋₆-aryl and (CH₂)₀₋₆-heteroaryl^{a}; or,
when -V-Z- is absent: U is absent, CH₂ or —CH₂CH₂—; X is CH₂; and Y is NH or N.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂—, wherein R18 is selected from:

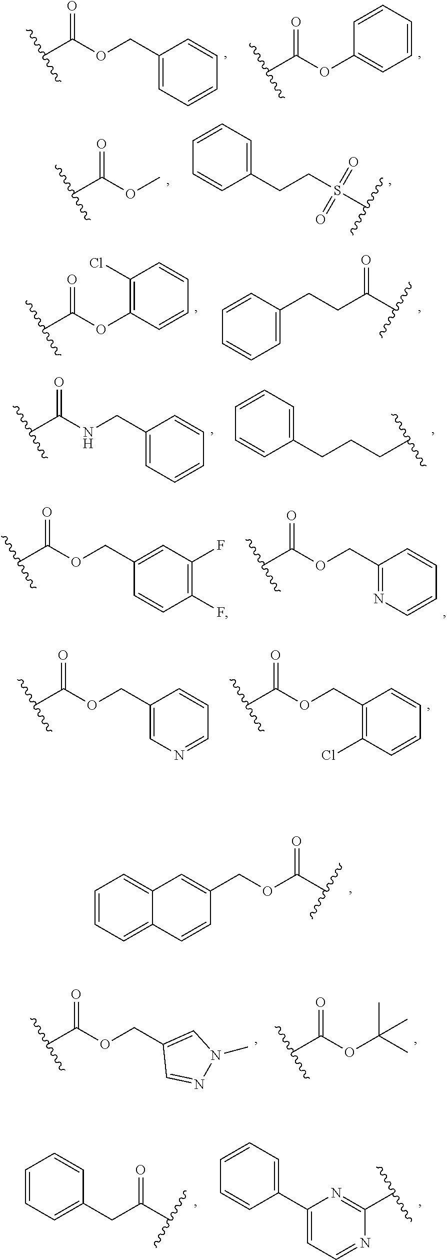

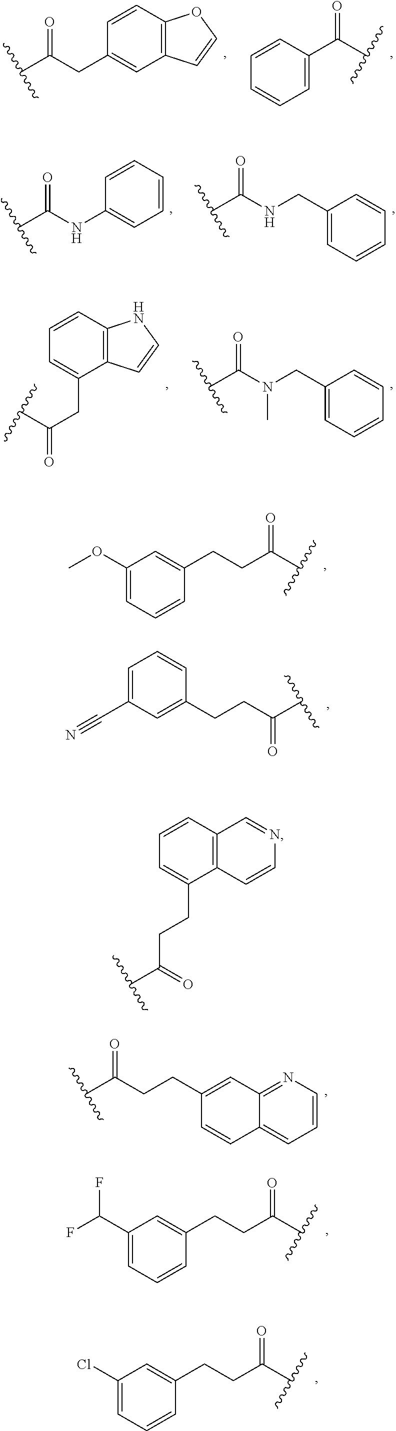

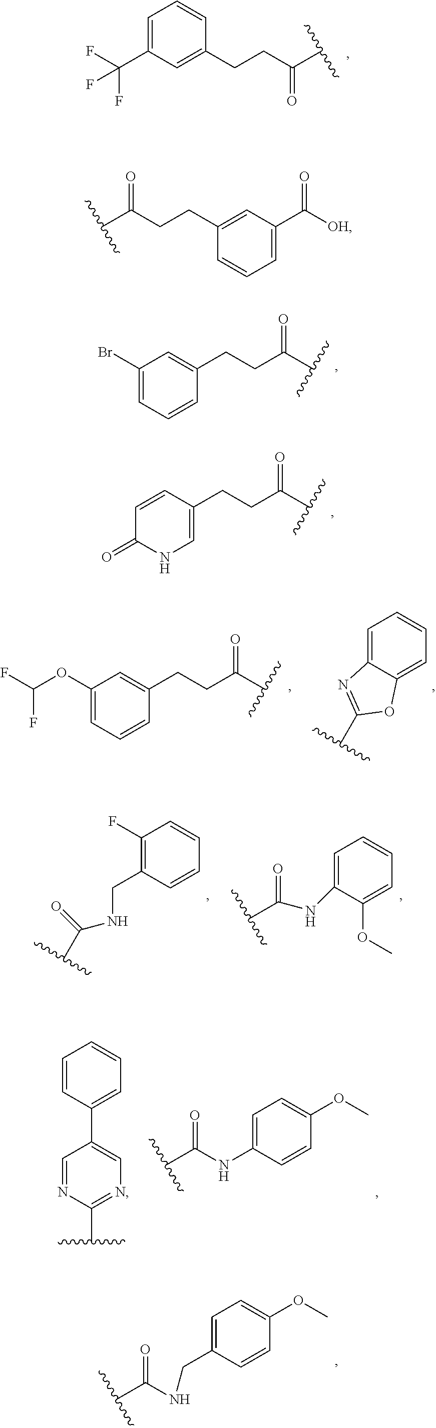

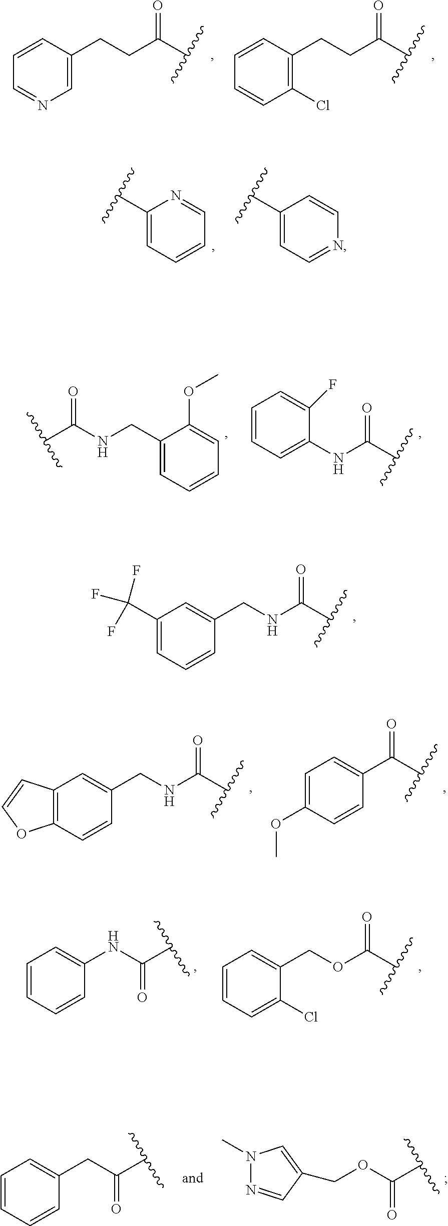

or,
when -V-Z- is absent: U is absent, CH₂ or —CH₂CH₂—; X is CH₂; and Y is NH or N.

Preferably, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂—, wherein R18 is selected from:

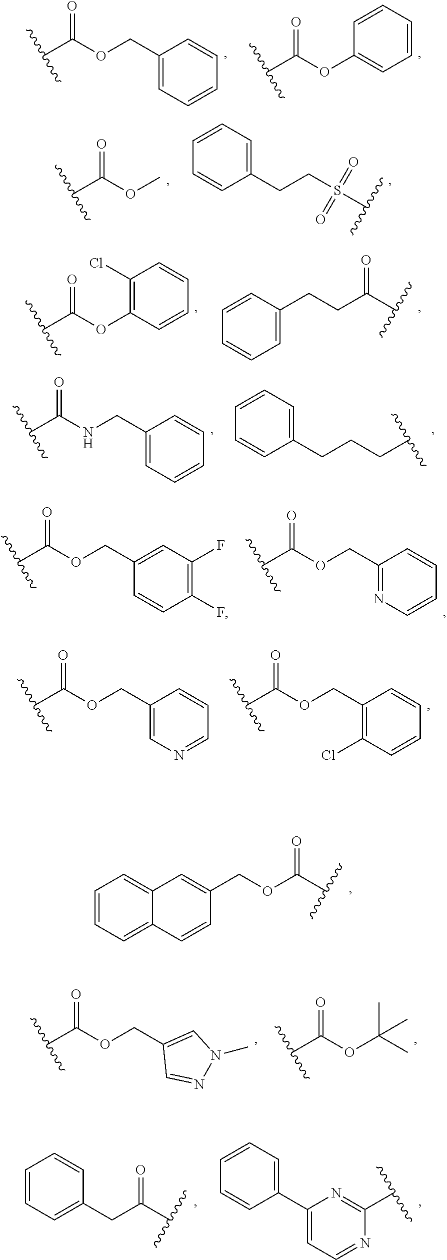

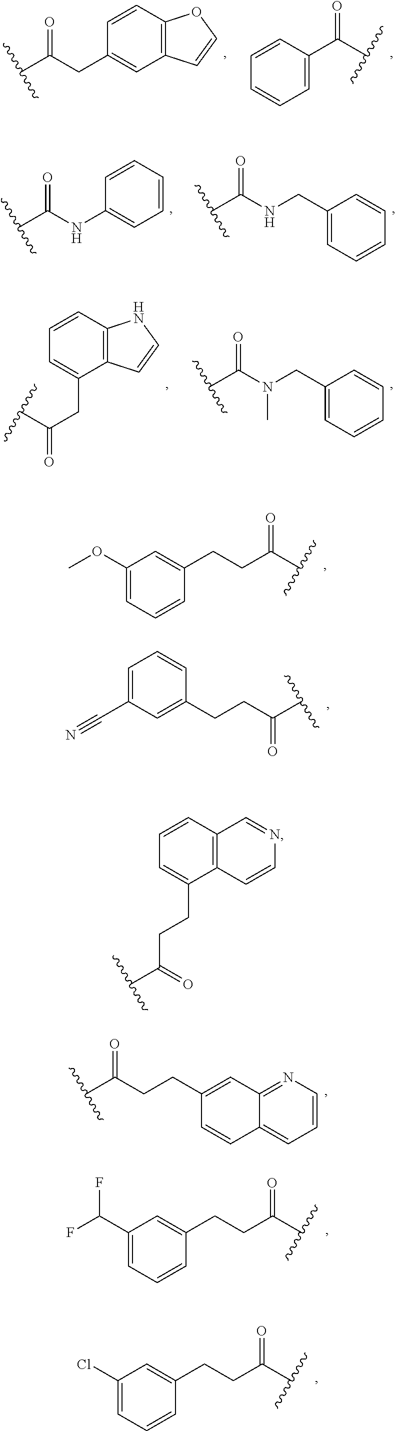

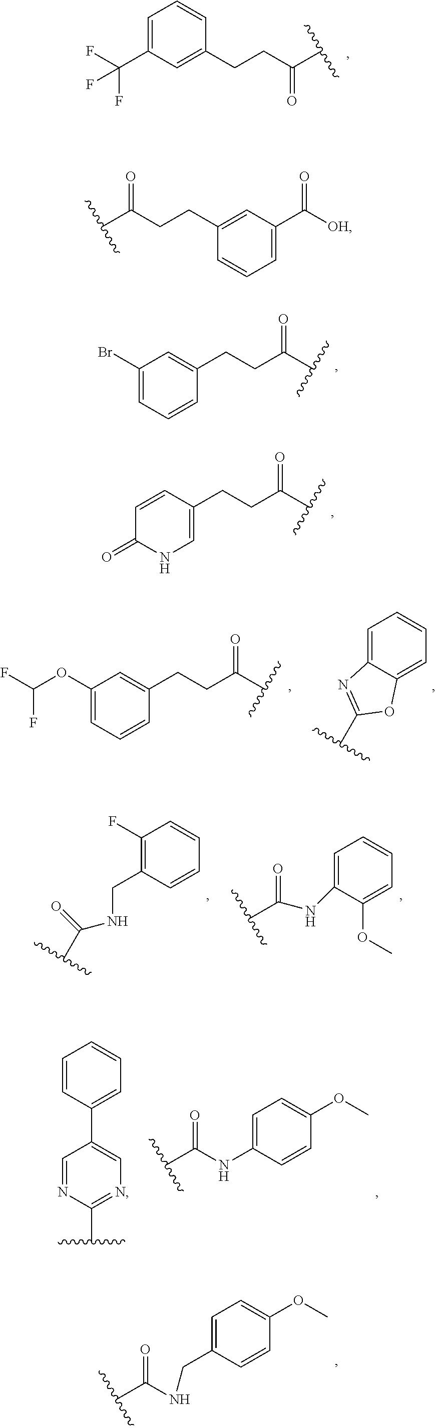

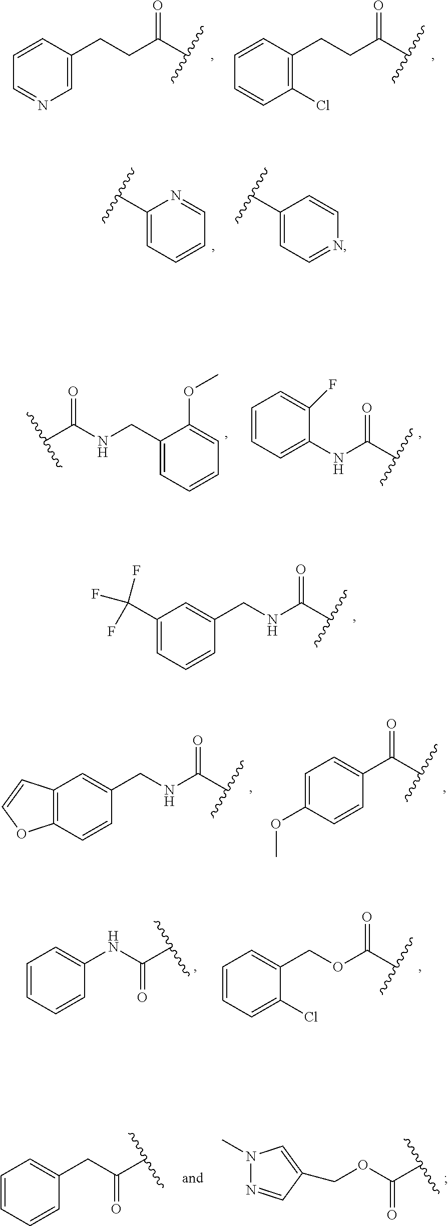

or,
when -V-Z- is absent: U is absent, CH₂ or —CH₂CH₂—; X is CH₂; and Y is NH or N.

Yet more preferably, -V-Z- is selected from —CH₂—, —O—CH, —CH₂—O—, —CH₂—CH₂—O—, —CH₂—N(R18)- and —N(R18)-CH₂, wherein R18 is selected from:
(CH₂)₀₋₆-aryl, (CH₂)₀₋₆-heteroary^{a}, C(═O)R19, C(═O)OR19, C(═O)NHR19, C(═O)N(alkyl)(R19), and SO₂R19;
   wherein R19 is selected from (CH₂)₀₋₆-aryl and (CH₂)-heteroary^{a};
X is CH₂; and Y is NH.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

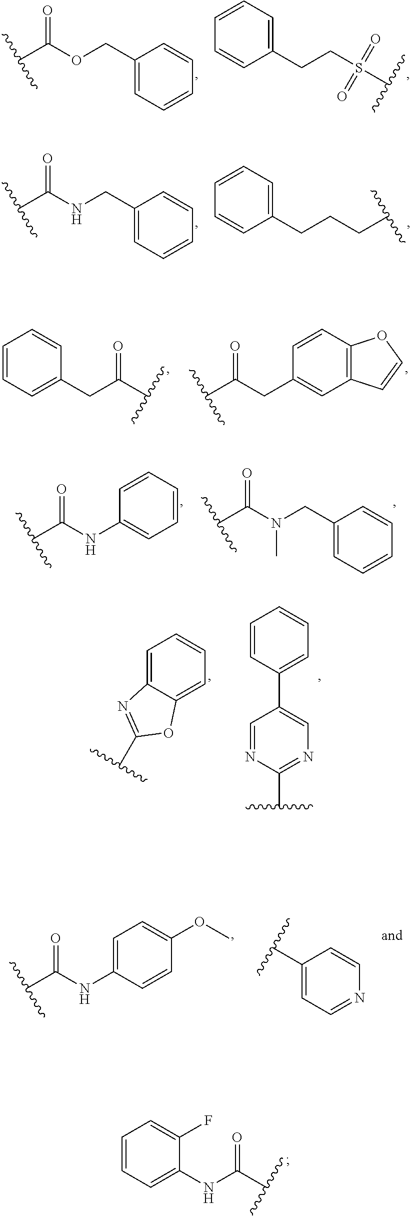

X is CH₂; and Y is NH.

More preferably, -V-Z- is selected from —CH₂—, —O—CH₂—, —CH₂—O— and —CH₂—CH₂—O—; X is CH₂; and Y is NH.

Alternatively, for compounds of formula (1d), X and Y are independently selected from O, CR1R2, C═O and NR12. When one of X or Y is C═O, the other is O, CR1R2 or NR12. When one of X or Y is NH, the other is CR1R2 or C═O. When one of X or Y is O, the other is CR1R2 or C═O.

X can be CR1R2. Y can be CR1R2. X and Y can be CR1R2.

R1 can be H. R1 can be alkyl, for example small alkyl such as methyl or ethyl, which can be optionally substituted as for alkyl. R1 can be alkoxy, for example methoxy or ethoxy, which can be optionally substituted as for alkoxy. R1 can be OH. R1 can be halo, for example chloro. R1 can be NR13R14, for example NH₂.

R2 can be H. R2 can be alkyl, for example small alkyl such as methyl or ethyl, which can be optionally substituted as for alkyl. R2 can be alkoxy, for example methoxy or ethoxy, which can be optionally substituted as for alkyl. R2 can be OH. R2 can be halo, for example chloro. R2 can be NR13R14, for example NH₂.

R1 can be H and R2 can be alkyl, for example small alkyl such as methyl or ethyl, which can be optionally substituted as for alkyl. R2 can be alkoxy, for example methoxy or ethoxy, which can be optionally substituted as for alkyl. R2 can be OH. R2 can be halo, for example chloro. R2 can be NR13R14, for example NH₂.

At least one of R1 and R2 can be other than H. At least one of R1 and R2 can be H. Preferably, both R1 and R2 are H.

X can be NR12. Y can be NR12. When one of X or Y is NR12, the other is CR1R2 or C═O. X can be NR12 and Y can be CR1R2 (as defined above). Alternatively, X can be CR1R2 (as defined above) and Y can be NR12. X can be NR12 and Y can be C═O. Alternatively, Y can be NR12 and X can be C═O.

R12 can be alkyl, for example small alkyl such as methyl or ethyl, which can be optionally substituted as for alkyl. R12 can be cycloalkyl, for example cyclopropyl, which can be substituted as for cycloalkyl. Preferably, R12 is H.

X can be C═O. Y can be C═O. When one of X or Y is C═O, the other is O, CR1R2 or NR12. X can be C═O and Y can be CR1R2 (as defined above). Alternatively, X can be CR1R2 (as defined above) and Y can be C═O.

X can be O. Y can be O. When one of X or Y is O, the other is CR1R2 or C═O. X can be O and Y can be CR1R2 (as defined above). Alternatively, X can be CR1R2 (as defined above) and Y can be O. X can be O and Y can be C═O. Alternatively, X can be C═O and Y can be O.

Preferably, X is CH₂ and Y is NH.

As noted above, B can be selected from:
(i) heteroaryl^{a};
(ii) aryl;
(iii) a 5- to 6-membered non-aromatic heterocyclic ring containing one N ring member, which is unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃; and
(iv) a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring may be optionally substituted with 1, 2, or 3 substituted by up to three substituents independently selected from alkyl, alkoxy, OH, OCF₃, halo, oxo, CN, and CF₃, wherein the 6,5-bicyclic ring may be attached via the 6- or 5-membered ring.

B can be selected from heteroaryl^{a} and aryl.

B can be heteroaryl^{a} and Y can be attached to B at a carbon atom on the heteroaryl^{a} ring.

B can be heteroaryl^{a} and Y can be attached to B at a carbon atom on the heteroaryl^{a} ring, and the two ring atoms adjacent to the carbon atom on the heteroaryl^{a} ring to which Y attaches can both be carbon.

Preferably, B is heteroaryl^{a}. When B is heteroaryl^{a}, B can be substituted with NH₂, and optionally substituted with 1 or 2 further substituents as for heteroaryl^{a}. When B is heteroaryl^{a}, B can be substituted with halo (e.g. chloro), and optionally substituted with 1 or 2 further substituents as for heteroaryl^{a}. B can be substituted with NH₂ and halo (e.g. chloro), and optionally 1 further substituent as for heteroaryl^{a}.

When B is heteroaryl^{a}, B can be a 5, 6, 9 or 10 membered mono- or bi-cyclic aromatic ring, containing, where possible, 1, 2, 3 or 4 ring members independently selected from N, NR12, S and O; wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be a 5, 6, 9 or 10 membered mono- or bi-cyclic aromatic ring, containing, where possible, 1, 2, 3 or 4 ring members independently selected from N, NR12, S and O; wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be a 9 or 10 membered bi-cyclic aromatic ring, containing, where possible, 1, 2, 3 or 4 ring members independently selected from N, NR12, S and O, optionally substituted as for heteroaryl^{a}. B can be a 9 or 10 membered bi-cyclic aromatic ring, containing, where possible, 1, 2, 3 or 4 ring members independently selected from N, NR12, S and O, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be a 9 or 10 membered bi-cyclic aromatic ring, containing, where possible, 1, 2, 3 or 4 ring members independently selected from N, NR12, S and O, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be a 9 or 10 membered bi-cyclic aromatic ring, containing, where possible, 1 or 2 ring members independently selected from N, NR12, S and O, optionally substituted as for heteroaryl^{a}. B can be a 9 or 10 membered bi-cyclic aromatic ring, containing, where possible, 1 or 2 ring members independently selected from N, NR12, S and O, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be a 9 or 10 membered bi-cyclic aromatic ring, containing, where possible, 1 or 2 ring members independently selected from N, NR12, S and O, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be a 9 or 10 membered bi-cyclic aromatic ring, containing, where possible, 1 or 2 ring members independently selected from N and NR12, optionally substituted as for heteroaryl^{a}. B can be a 9 or 10 membered bi-cyclic aromatic ring, containing, where possible, 1 or 2 ring members independently selected from N and NR12, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be a 9 or 10 membered bi-cyclic aromatic ring, containing, where possible, 1 or 2 ring members independently selected from N and NR12, wherein B may be optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B is preferably isoquinolinyl or azaindole, optionally substituted as for heteroaryl^{a}. B is preferably isoquinolinyl or azaindole, optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B is preferably isoquinolinyl or azaindole, optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃. B is preferably isoquinolinyl or azaindole, optionally substituted with 1, 2 or 3 substituents independently selected from alkyl, halo, and —(CH₂)₀₋₃—NR13R14.

When B is heteroaryl^{a}, B is preferably isoquinolinyl or azaindole (specifically 7-azaindole), optionally substituted as for heteroaryl^{a}. B is preferably isoquinolinyl or azaindole (specifically 7-azaindole), optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B is preferably isoquinolinyl or azaindole (specifically 7-azaindole), optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B is preferably isoquinolinyl substituted with —(CH₂)₀₋₃—NR13R14 or azaindole optionally substituted as for heteroaryl^{a}. B is preferably isoquinolinyl substituted with —NR13R14 or azaindole optionally substituted as for heteroaryl^{a}. B is preferably isoquinolinyl substituted with —NH₂ or azaindole optionally substituted as for heteroaryl^{a}. B is preferably isoquinolinyl substituted with —NH₂ or azaindole optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B is preferably isoquinolinyl substituted with —NH₂ or azaindole optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃. B is preferably isoquinolinyl substituted with —NH₂ or azaindole optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B is preferably isoquinolinyl substituted with —(CH₂)₀₋₃—NR13R14 or azaindole (specifically 7-azaindole) optionally substituted as for heteroaryl^{a}. B is preferably isoquinolinyl substituted with —NR13R14 or 7-azaindole optionally heteroaryl^{a}. B is preferably isoquinolinyl substituted with —NH₂ or azaindole (specifically 7-azaindole) optionally substituted as for heteroaryl^{a}. B is preferably isoquinolinyl substituted with —NH₂ or azaindole (specifically 7-azaindole) optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B is preferably isoquinolinyl substituted with —NH₂ or azaindole (specifically 7-azaindole) optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be selected from isoquinolinyl

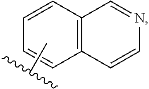

optionally substituted as for heteroaryl^{a}; 6-azaindolyl

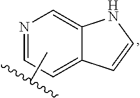

optionally substituted as for heteroaryl^{a}; and 7-azaindolyl

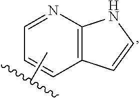

optionally substituted as for heteroaryl^{a}. B can be isoquinolinyl

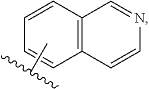

optionally substituted as for heteroaryl^{a}. B can be 6-azaindolyl

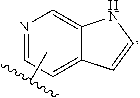

optionally substituted as for heteroaryl^{a}. B can be 7-azaindolyl

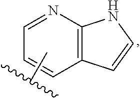

optionally substituted as for heteroaryl^{a}.

When B is heteroaryl^{a}, B is preferably selected from isoquinolinyl

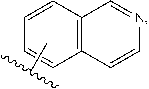

optionally substituted as for heteroaryl^{a}; and 7-azaindolyl

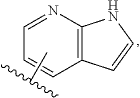

optionally substituted as for heteroaryl^{a}.

When B is heteroaryl^{a}, B is preferably isoquinolinyl or azaindole, wherein Y is attached to B at a carbon atom on the heteroaryl^{a} ring. When B is heteroaryl^{a}, B is preferably isoquinolinyl or azaindole, wherein Y is attached to B at a carbon atom on the heteroaryl^{a} ring, and the two ring atoms adjacent to the carbon atom on the heteroaryl^{a} ring to which Y attaches are both carbon.

When B is heteroaryl^{a}, B is preferably isoquinolinyl or azaindole (specifically 7-azaindole), wherein Y is attached to B at a carbon atom on the heteroaryl^{a} ring. When B is heteroaryl^{a}, B is preferably isoquinolinyl or azaindole, wherein Y is attached to B at a carbon atom on the heteroaryl^{a} ring, and the two ring atoms adjacent to the carbon atom on the heteroaryl^{a} ring to which Y attaches are both carbon.

It will be understood that, in the instance when Y is attached to B at a carbon atom on the heteroaryl^{a} ring, the attachment of Y to B can be at any carbon on the heteroaryl^{a} ring, so long as the remainder of the ring is still a heteroaryl ring. For example, if B is 7-azaindole, the attachment to Y can be at any of the following ring atoms:

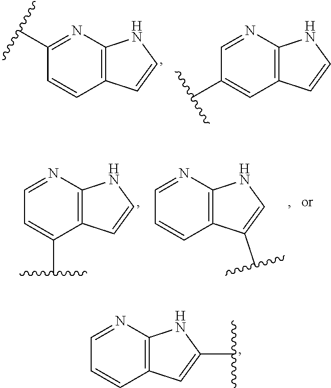

but not at a nitrogen ring atom:

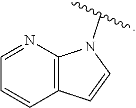 [SMILES: *n1ccc2cccnc21]

It will be understood that, in the instance when Y is attached to B at a carbon atom on the heteroaryl^{a} ring, and the two ring atoms adjacent to the carbon atom on the heteroaryl^{a} ring to which Y attaches are both carbon, these adjacent ring atoms can be, where possible, substituted or unsubstituted as defined in the embodiment or claim. Further, for example, if B is 7-azaindole, the attachment to Y can be at any of the following ring atoms:

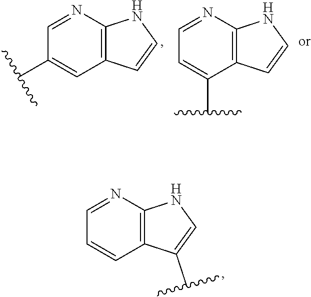

but not at the following ring atoms:

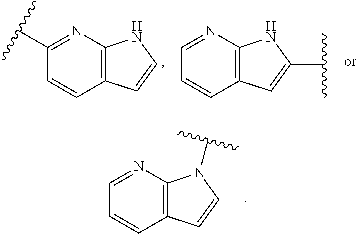

B can be selected from:
isoquinolinyl, selected from

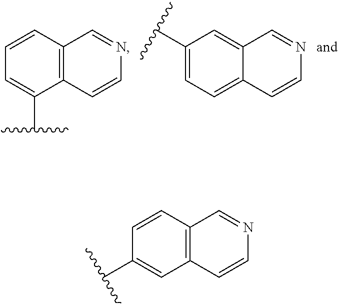

optionally substituted as for heteroaryl^{a};
7-azaindolyl optionally substituted as for heteroaryl^{a}; and

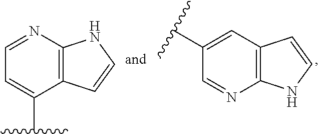 [SMILES: *c1ccnc2[nH]ccc12 ; *c1cnc2[nH]ccc2c1]

6-azaindolyl

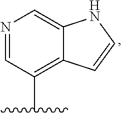 [SMILES: *c1cncc2[nH]ccc12]

optionally substituted as for heteroaryl^{a}.

B can preferably be selected from:
isoquinolinyl, selected from

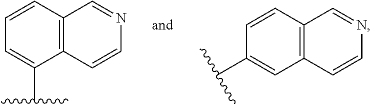 [SMILES: *c1cccc2cnccc12 ; "and" ; *c1ccc2cnccc2c1]

optionally substituted as for heteroaryl^{a}; and
7-azaindolyl

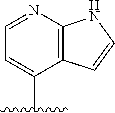 [SMILES: *c1ccnc2[nH]ccc12]

optionally substituted as for heteroaryl^{a}.

When B is isoquinolinyl or azaindole, B can be selected from

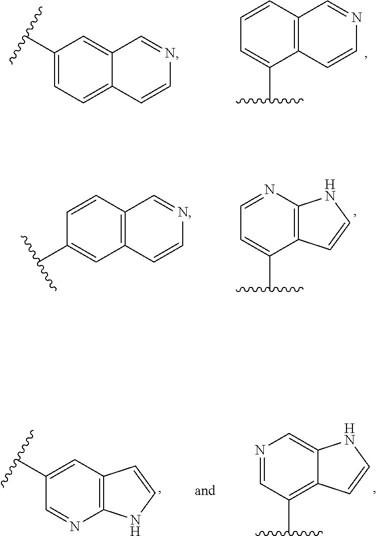

optionally substituted as for heteroaryl^{a}.

When B is isoquinolinyl or azaindole, B is preferably selected from

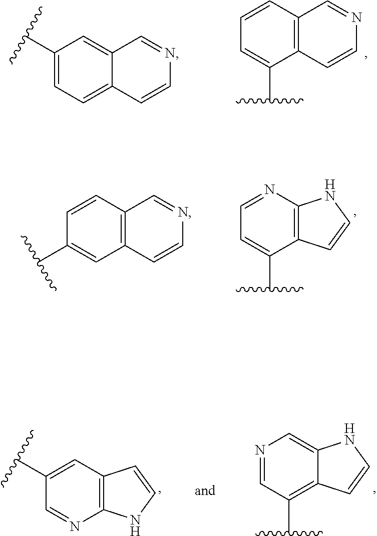

optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is isoquinolinyl or azaindole, B is preferably selected from

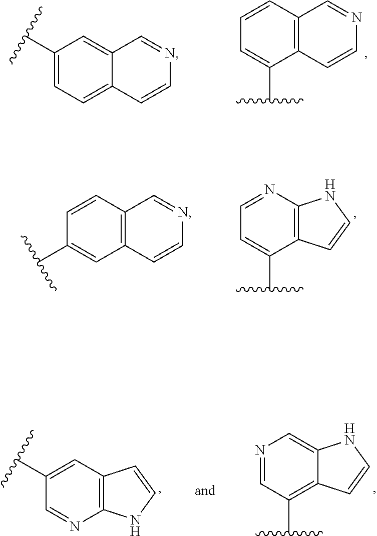

optionally substituted with 1, 2 or 3 substituents independently selected from alkyl, halo, and —(CH₂)₀₋₃—NR13R14.

When B is isoquinolinyl or azaindole, B is preferably selected from

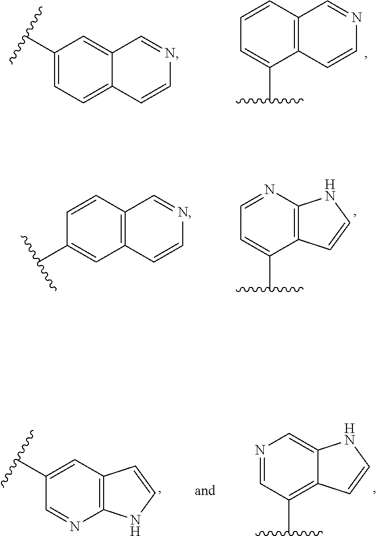

optionally substituted with 1, 2 or 3 substituents independently selected from alkyl, halo, and —NH₂.

More specifically, B is selected from isoquinolinyl, selected from

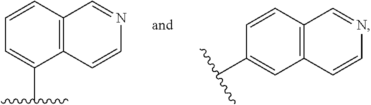 [SMILES: *c1cccc2cnccc12 ; "and" ; *c1ccc2cnccc2c1]

substituted with NH₂, optionally further substituted with 1 or 2 substituents as for heteroaryl^{a}; 6-azaindolyl

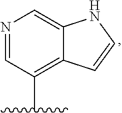 [SMILES: *c1cncc2[nH]ccc12]

optionally substituted as for heteroaryl^{a}; and 7-azaindolyl selected from

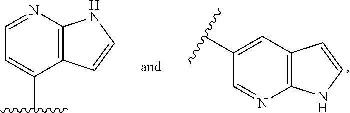 [SMILES: *c1ccnc2[nH]ccc12 ; "and" ; *c1cnc2[nH]ccc2c1]

optionally substituted as for heteroaryl^{a}. B can be isoquinolinyl, selected from

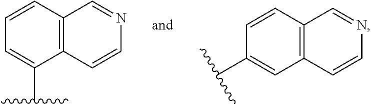 [SMILES: *c1cccc2cnccc12 ; "and" ; *c1ccc2cnccc2c1]

substituted with NH₂, optionally further substituted with 1 or 2 substituents as for heteroaryl^{a}. B can be 6-azaindolyl

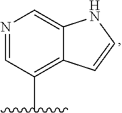 [SMILES: *c1cncc2[nH]ccc12]

optionally substituted as for heteroaryl^{a}. B can be 7-azaindolyl selected from

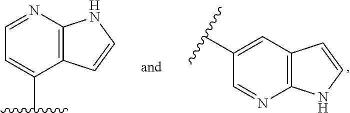 [SMILES: *c1ccnc2[nH]ccc12 ; "and" ; *c1cnc2[nH]ccc2c1]

optionally substituted as for heteroaryl^{a}.

More specifically, B is selected from isoquinolinyl, selected from

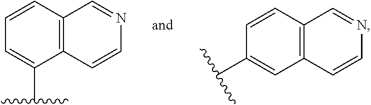 [SMILES: *c1cccc2cnccc12 ; "and" ; *c1ccc2cnccc2c1]

substituted with NH₂, optionally further substituted with 1 or 2 substituents as for heteroaryl^{a}; and 7-azaindolyl

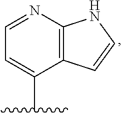 [SMILES: *c1ccnc2[nH]ccc12]

optionally substituted as for heteroaryl^{a}.

Yet more specifically, B is selected from: isoquinolinyl, substituted with NH₂ at the 1-position

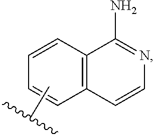

optionally further substituted with 1 or 2 substituents as for heteroaryl^{a}; 6-azaindolyl

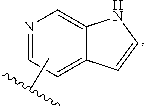

optionally substituted as for heteroaryl^{a}; and 7-azaindolyl

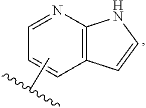

optionally substituted as for heteroaryl^{a}. B can be isoquinolinyl, substituted with NH₂ at the 1-position

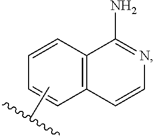

optionally further substituted with 1 or 2 substituents as for heteroaryl^{a}. B can be 6-azaindolyl

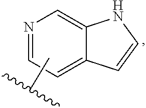

optionally substituted as for heteroaryl^{a}. B can be 7-azaindolyl

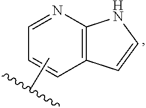

optionally substituted as for heteroaryl^{a}.

Yet more specifically, B is selected from: isoquinolinyl, substituted with NH₂ at the 1-position

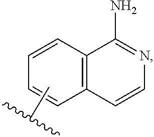

optionally further substituted with 1 or 2 substituents as for heteroaryl^{a}; and 7-azaindolyl,

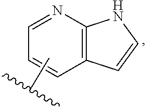

optionally substituted as for heteroaryl^{a}.

Preferably, when B is heteroaryl^{a}, B is selected from: isoquinolinyl, substituted with NH₂ at the 1-position, selected from

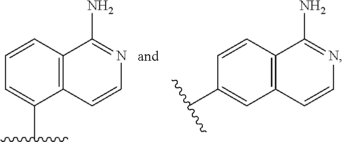 [SMILES: *c1cccc2c(N)nccc12 ; "and" ; *c1ccc2c(N)nccc2c1]

optionally further substituted with 1 or 2 substituents as for heteroaryl^{a}; 6-azaindolyl

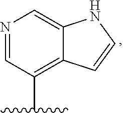 [SMILES: *c1cncc2[nH]ccc12]

optionally substituted as for heteroaryl^{a}; and 7-azaindolyl selected from

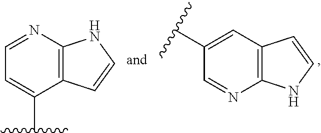 [SMILES: *c1ccnc2[nH]ccc12 ; *c1cnc2[nH]ccc2c1]

optionally substituted as for heteroaryl^{a}.

Preferably, when B is heteroaryl^{a}, B is selected from: isoquinolinyl, substituted with NH₂ at the 1-position, selected from

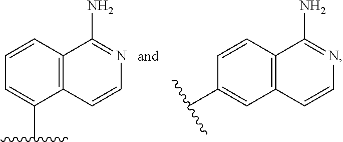 [SMILES: *c1cccc2c(N)nccc12 ; "and" ; *c1ccc2c(N)nccc2c1]

optionally further substituted with 1 or 2 substituents as for heteroaryl^{a}; and 7-azaindolyl selected from

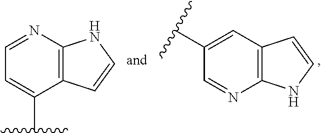 [SMILES: *c1ccnc2[nH]ccc12 ; *c1cnc2[nH]ccc2c1]

optionally substituted as for heteroaryl^{a}.

Preferably, when B is heteroaryl^{a}, B is selected from: isoquinolinyl, substituted with NH₂ at the 1-position, selected from

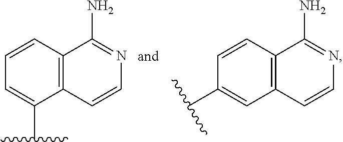 [SMILES: *c1cccc2c(N)nccc12 ; "and" ; *c1ccc2c(N)nccc2c1]

optionally further substituted with 1 or 2 substituents as for heteroaryl^{a}; and 7-azaindolyl

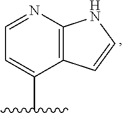 [SMILES: *c1ccnc2[nH]ccc12]

optionally substituted as for heteroaryl^{a}.

Specifically, B can be isoquinolinyl, substituted with NH₂ at the 1-position, selected from

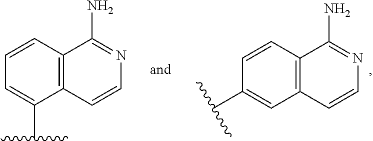 [SMILES: *c1cccc2c(N)nccc12 ; *c1ccc2c(N)nccc2c1]

optionally further substituted with 1 or 2 substituents as for heteroaryl^{a}. B can be isoquinolinyl, substituted with NH₂ at the 1-position

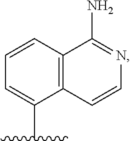 [SMILES: *c1cccc2c(N)nccc12]

optionally further substituted with 1 or 2 substituents as for heteroaryl^{a}. B can be isoquinolinyl, substituted with NH₂ at the 1-position

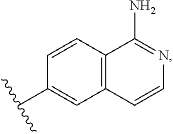 [SMILES: *c1ccc2c(N)nccc2c1]

optionally further substituted with 1 or 2 substituents as for heteroaryl^{a}. B can be 6-azaindolyl

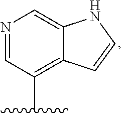 [SMILES: *c1cncc2[nH]ccc12]

optionally substituted as for heteroaryl^{a}. B can be 7-azaindolyl

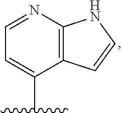 [SMILES: *c1ccnc2[nH]ccc12]

optionally substituted as for heteroaryl^{a}.

When B is isoquinolinyl or azaindole B can be selected from

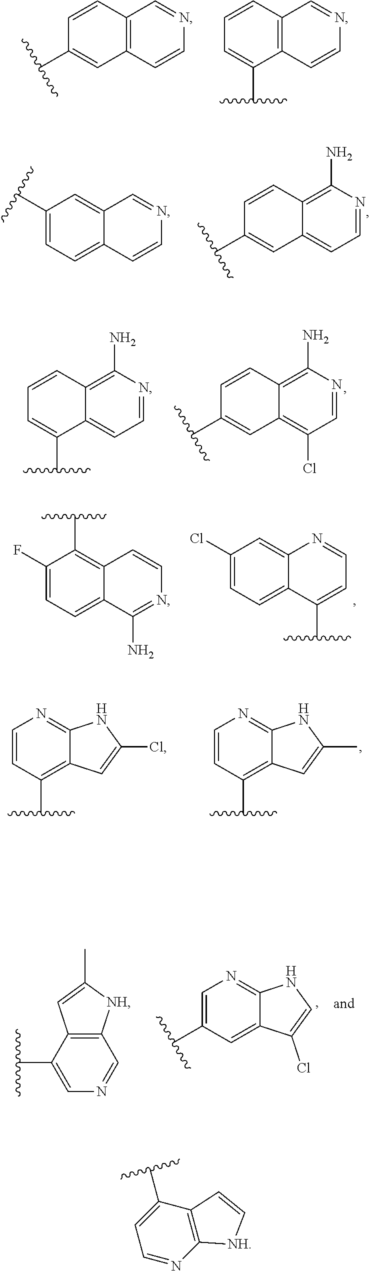

When B is isoquinolinyl, B can be selected from

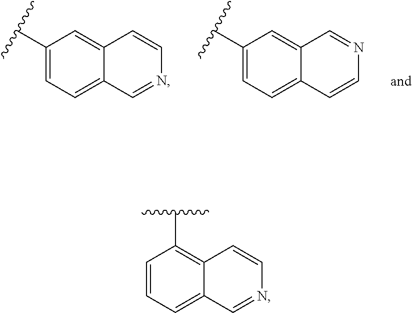 [SMILES: *c1ccc2cnccc2c1 ; *c1ccc2ccncc2c1 ; "and" ; *c1cccc2cnccc12]

optionally substituted as for heteroaryl^{a}. B can be selected from

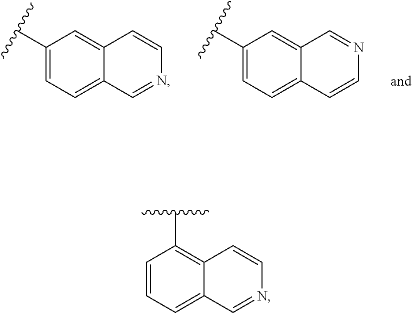 [SMILES: *c1ccc2cnccc2c1 ; *c1ccc2ccncc2c1 ; "and" ; *c1cccc2cnccc12]

optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be selected from

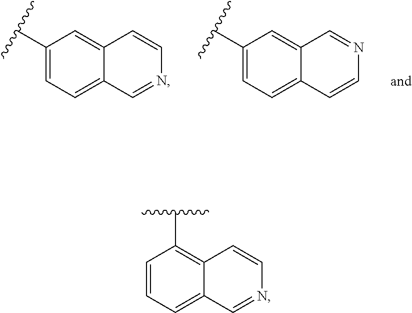 [SMILES: *c1ccc2cnccc2c1 ; *c1ccc2ccncc2c1 ; "and" ; *c1cccc2cnccc12]

optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is isoquinolinyl, B can be selected from

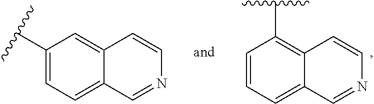 [SMILES: *c1ccc2cnccc2c1 ; *c1cccc2cnccc12]

optionally substituted as for heteroaryl^{a}. B can be selected from

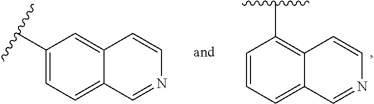 [SMILES: *c1ccc2cnccc2c1 ; *c1cccc2cnccc12]

optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be selected from

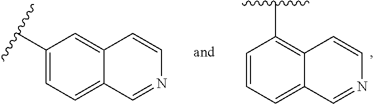 [SMILES: *c1ccc2cnccc2c1 ; *c1cccc2cnccc12]

optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is isoquinolinyl, B can be

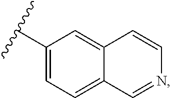 [SMILES: *c1ccc2cnccc2c1]

optionally substituted as for heteroaryl^{a}. B can be

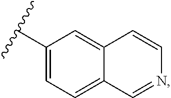 [SMILES: *c1ccc2cnccc2c1]

optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be

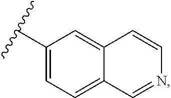 [SMILES: *c1ccc2cnccc2c1]

optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is isoquinolinyl, B can be

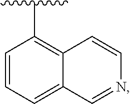 [SMILES: *c1cccc2cnccc12]

optionally substituted as for heteroaryl^{a}. B can be

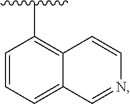 [SMILES: *c1cccc2cnccc12]

optionally substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be

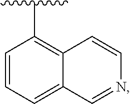 [SMILES: *c1cccc2cnccc12]

optionally substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B is preferably isoquinolinyl, substituted with NH₂, and optionally substituted with 1 or 2 further substituents as for heteroaryl^{a}. B is preferably isoquinolinyl, substituted with NH₂, and optionally substituted with 1, or 2 further substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B is preferably isoquinolinyl, substituted with NH₂, and optionally substituted with 1, or 2 further substituents independently selected from alkyl, alkoxy, OH, OCF₃, halo, aryl^{b}, —(CH₂)₀₋₃—NR13R14, heteroaryl^{b} and CF₃.

When B is isoquinolinyl, substituted with NH₂, B can be selected from

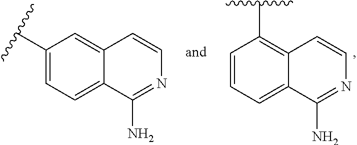 [SMILES: *c1ccc2c(N)nccc2c1 ; *c1cccc2c(N)nccc12]

optionally substituted with 1 or 2 further substituents as for heteroaryl^{a}. B can be selected from

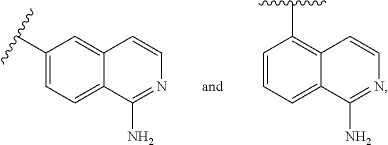 [SMILES: *c1ccc2c(N)nccc2c1 ; "and" ; *c1cccc2c(N)nccc12]

optionally substituted with 1, or 2 further substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be selected from

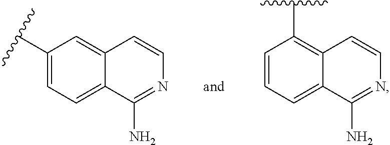 [SMILES: *c1ccc2c(N)nccc2c1 ; "and" ; *c1cccc2c(N)nccc12]

optionally substituted with 1, or 2 further substituents independently selected from alkyl, alkoxy, OH, OCF₃, halo, aryl^{b}, —(CH₂)₀₋₃—NR13R14, heteroaryl^{b} and CF₃.

When B is isoquinolinyl, substituted with NH₂, B can be

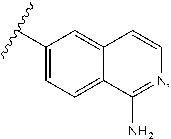 [SMILES: *c1ccc2c(N)nccc2c1]

optionally substituted with 1 or 2 further substituents as for heteroaryl^{a}. B can be

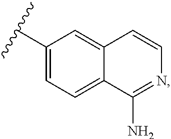 [SMILES: *c1ccc2c(N)nccc2c1]

optionally substituted with 1, or 2 further substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be

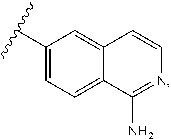 [SMILES: *c1ccc2c(N)nccc2c1]

optionally substituted with 1, or 2 further substituents independently selected from alkyl, alkoxy, OH, OCF₃, halo, aryl^{b}, —(CH₂)₀₋₃—NR13R14, heteroaryl^{b} and CF₃.

When B is isoquinolinyl, substituted with NH₂, B can be

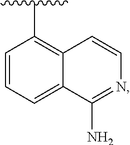 [SMILES: *c1cccc2c(N)nccc12]

optionally substituted with 1 or 2 further substituents as for heteroaryl^{a}. B can be

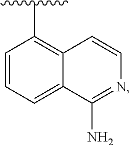 [SMILES: *c1cccc2c(N)nccc12]

optionally substituted with 1, or 2 further substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be

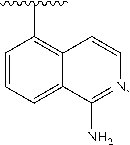 [SMILES: *c1cccc2c(N)nccc12]

optionally substituted with 1, or 2 further substituents independently selected from alkyl, alkoxy, OH, OCF₃, halo, aryl^{b}, —(CH₂)₀₋₃—NR13R14, heteroaryl^{b} and CF₃.

When B is isoquinolinyl, substituted with NH₂, B can be selected from

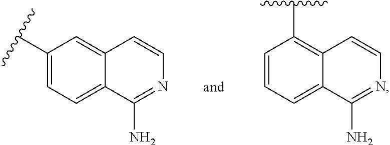 [SMILES: *c1ccc2c(N)nccc2c1 ; "and" ; *c1cccc2c(N)nccc12]

optionally substituted with a further substituent selected from halo.

When B is isoquinolinyl, substituted with NH₂, B can be

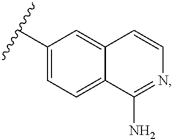 [SMILES: *c1ccc2c(N)nccc2c1]

optionally substituted with a further substituent selected from halo (e.g. chloro).

When B is isoquinolinyl, substituted with NH₂, B can be

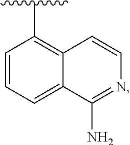 [SMILES: *c1cccc2c(N)nccc12]

optionally substituted with a further substituent selected from halo (e.g. chloro).

When B isoquinolinyl, substituted with NH₂, B can be selected from

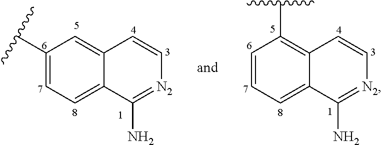

optionally substituted with a further substituent selected from halo (e.g. chloro) at the carbon marked as 4.

When B is isoquinolinyl, substituted with NH₂, B can be

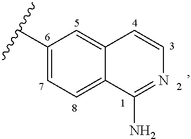 [SMILES: *c1ccc2c(N)nccc2c1]

optionally substituted with a further substituent selected from halo (e.g. chloro) at the carbon marked as 4.

When B is isoquinolinyl, substituted with NH₂, B can be

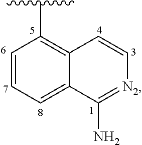

optionally substituted with a further substituent selected from halo (e.g. chloro), at the carbon marked as 4.

Preferably, B is selected from:

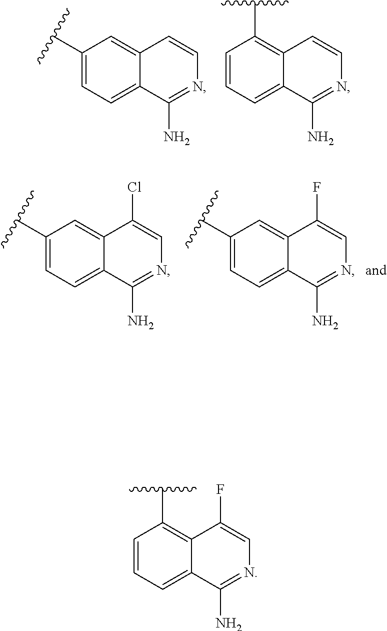

More preferably, B is selected from:

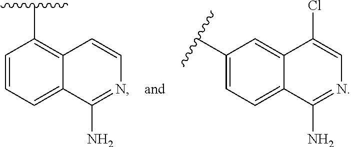 [SMILES: *c1cccc2c(N)nccc12 ; *c1ccc2c(N)ncc(Cl)c2c1]

When B is heteroaryl^{a}, B can be a 5, 6, 9 or 10 membered mono- or bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃.

When B is heteroaryl^{a}, B can be a 9 or 10 membered bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃

When B is heteroaryl^{a}, B can be a 5, 6, 9 or 10 membered mono- or bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃.

When B is heteroaryl^{a}, B can be a 9 or 10 membered bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃.

When B is heteroaryl^{a}, B can be a 5, 6, 9 or 10 membered mono- or bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be a 9 or 10 membered bi-cyclic aromatic ring, containing, where possible, 1, 2, 3 or 4 ring members independently selected from N, NR12, S and O which is substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be quinolinyl or isoquinolinyl which is substituted with 1, 2 or 3 substituents independently selected from alkyl, alkoxy, OH, OCF₃, CN, halo, aryl^{b}, —(CH₂)₀₋₃—NR13R14, heteroaryl^{b}, —C(═O)OR12, —C(═O)NR13R14 and CF₃. B can be quinolinyl or isoquinolinyl which is substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be quinolinyl or isoquinolinyl which is substituted with 1, 2 or 3 substituents independently selected from alkyl, alkoxy, OH, OCF₃, halo, aryl^{b}, —(CH₂)₀₋₃—NR13R14, heteroaryl^{b} and CF₃. When B is heteroaryl^{a}, B can be isoquinolinyl which is substituted with 1, 2 or 3 substituents independently selected from alkyl, alkoxy, OH, OCF₃, CN, halo, aryl^{b}, —(CH₂)₀₋₃—NR13R14, heteroaryl^{b}, —C(═O)OR12, —C(═O)NR13R14 and CF₃. B can be isoquinolinyl which is substituted with 1, 2 or 3 substituents independently selected from alkyl, alkoxy, OH, OCF₃, halo, aryl^{b}, —(CH₂)₀₋₃—NR13R14, heteroaryl^{b}, —C(═O)OR12, —C(═O)NR13R14 and CF₃. B can be isoquinolinyl which is substituted with 1, 2 or 3 substituents independently selected from alkyl, alkoxy, OH, OCF₃, halo, aryl^{b}, —(CH₂)₀₋₃—NR13R14, heteroaryl^{b} and CF₃.

When B is heteroaryl^{a}, B can be isoquinolinyl, optionally substituted as for heteroaryl^{a}.

When B is heteroaryl^{a}, B can be isoquinolinyl substituted with 1, 2 or 3 substituents independently selected from alkyl, alkoxy, OH, OCF₃, halo, CN, aryl^{b}, —(CH₂)₁₋₃—NR13R14, heteroaryl^{b}, —C(═O)OR12, —C(═O)NR13R14 and CF₃.

When B is heteroaryl^{a}, B can be isoquinolinyl substituted with 1, 2 or 3 substituents independently selected from alkoxy.

When B is heteroaryl^{a}, B can be isoquinolinyl substituted with 1, 2 or 3 substituents selected from —OMe.

When B is heteroaryl^{a}, B can be isoquinolinyl substituted with —OMe. B can be selected from:

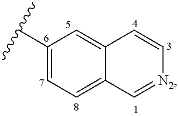

substituted with —OMe at one of the carbons marked as 3, 4, 5, 7 or 8; and

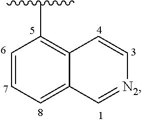

substituted with —OMe at one of the carbons marked as 3, 4, 6, 7 or 8. B can be selected from

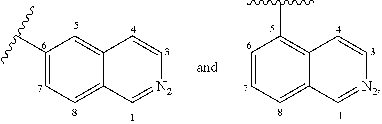

substituted with —OMe at the carbon marked as 8. B can be

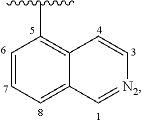

substituted with —OMe at one of the carbons marked as 3, 4, 6, 7 or 8. B can be

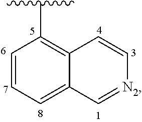

substituted with —OMe at the carbon marked as 8. B can be

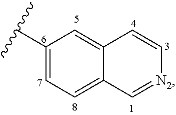

substituted with —OMe at one of the carbons marked as 3, 4, 5, 7 or 8. B can be

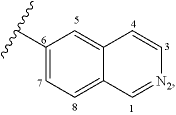

substituted with —OMe at the carbon marked as 8.

When B is heteroaryl^{a}, B can be isoquinolinyl substituted with —Me. B can be selected from:

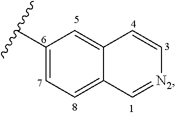

substituted with —Me at one of the carbons marked as 3, 4, 5, 7 or 8; and

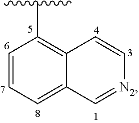

substituted with —Me at one of the carbons marked as 3, 4, 6, 7 or 8. B can be selected from

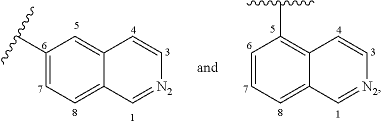

substituted with —Me at the carbon marked as 8. B can be

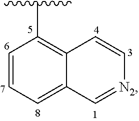

substituted with —Me at one of the carbons marked as 3, 4, 6, 7 or 8. B can be

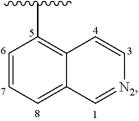

substituted with —Me at the carbon marked as 8. B can be

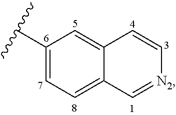

substituted with —Me at one of the carbons marked as 3, 4, 5, 7 or 8. B can be

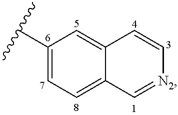

substituted with —Me at the carbon marked as 8.

When B is heteroaryl^{a}, B can be a 9-membered, bi-cyclic aromatic ring containing 1 or 2 ring members independently selected from N, NR12, S and O; wherein B may be optionally substituted as for heteroaryl^{a}.

When B is heteroaryl^{a}, B can be a 9-membered, bi-cyclic aromatic ring containing 1 or 2 ring members independently selected from N, NR12, S and O; wherein B is substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃; wherein the substituents on B are attached to carbon ring members only.

As noted above, B can preferably be azaindole, optionally substituted as for heteroaryl^{a}. Specifically, B can be selected from 4-azaindole, 5-azaindole, 6-azaindole and 7-azaindole, each optionally substituted as for heteroaryl^{a}. Preferably, B is 7-azaindole.

B can be 7-azaindole optionally substituted as for heteroaryl^{a}.

When B is azaindole, B can be selected from

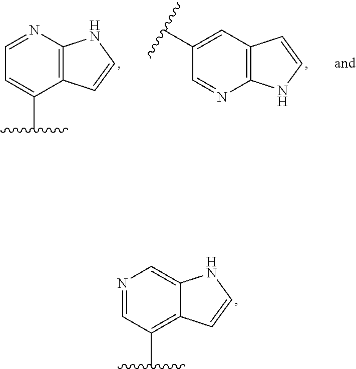 [SMILES: *c1ccnc2[nH]ccc12 ; *c1cnc2[nH]ccc2c1 ; *c1cncc2[nH]ccc12]

optionally substituted as for heteroaryl^{a}.

When B is 7-azaindole, B can be selected from

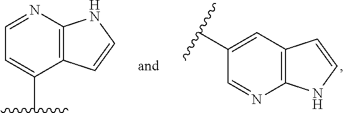 [SMILES: *c1ccnc2[nH]ccc12 ; *c1cnc2[nH]ccc2c1]

optionally substituted as for heteroaryl^{a}.

When B is 7-azaindole, B can be

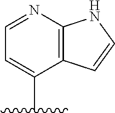 [SMILES: *c1ccnc2[nH]ccc12]

optionally substituted as for heteroaryl^{a}.

When B is azaindole, B can be selected from

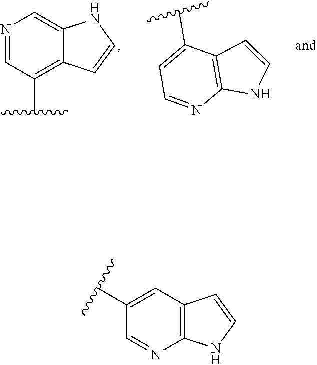 [SMILES: *c1cncc2[nH]ccc12 ; *c1ccnc2[nH]ccc12 ; *c1cnc2[nH]ccc2c1]

substituted with methyl, and optionally substituted with 1 or 2 further substituents as for heteroaryl^{a}.

When B is azaindole (particularly 7-azaindole), B can be substituted with NH₂, and optionally substituted with 1 or 2 further substituents as for heteroaryl^{a}. When B is azaindole (particularly 7-azaindole), B can be substituted with alkyl, and optionally substituted with 1 or 2 further substituents as for heteroaryl^{a}. For example, when B is azaindole (particularly 7-azaindole), B can be substituted with methyl, and optionally substituted with 1 or 2 further substituents as for heteroaryl^{a}. When B is azaindole (particularly 7-azaindole), B can be substituted with halo (e.g. chloro), and optionally substituted with 1 or 2 further substituents as for heteroaryl^{a}. When B is azaindole (particularly 7-azaindole), B can be substituted with NH₂ and halo (e.g. chloro), and optionally substituted with 1 or 2 further substituents as for heteroaryl^{a}.

For example, B can be selected from:

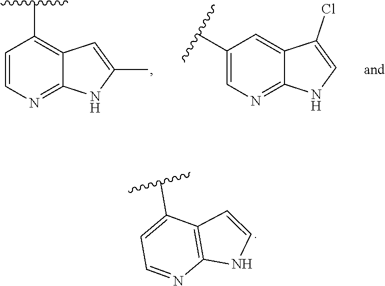 [SMILES: *c1ccnc2[nH]c(C)cc12 ; *c1cnc2[nH]cc(Cl)c2c1 ; *c1ccnc2[nH]ccc12]

When B is azaindole (particularly 7-azaindole), B can be substituted with NH₂, and optionally substituted with 1 or 2 further substituents as for heteroaryl^{a}. When B is azaindole (particularly 7-azaindole), B can be substituted with halo (e.g. chloro), and optionally substituted with 1 or 2 further substituents as for heteroaryl^{a}. When B is azaindole (particularly 7-azaindole), B can be substituted with NH₂ and halo (e.g. chloro), and optionally substituted with 1 or 2 further substituents as for heteroaryl^{a}. For example, B can be selected from:

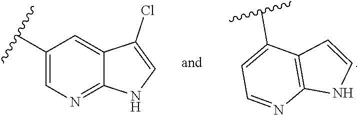 [SMILES: *c1cnc2[nH]cc(Cl)c2c1 ; *c1ccnc2[nH]ccc12]

When B is 7-azaindole, B can be substituted with methyl, and optionally substituted with 1 or 2 further substituents as for heteroaryl^{a}.

When B is 7-azaindole, B can be selected from

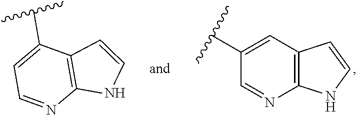 [SMILES: *c1ccnc2[nH]ccc12 ; *c1cnc2[nH]ccc2c1]

substituted with methyl, and optionally substituted with 1 or 2 further substituents as for heteroaryl^{a}.

When B is 7-azaindole, B can be

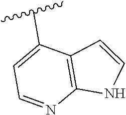 [SMILES: *c1ccnc2[nH]ccc12]

substituted with methyl, and optionally substituted with 1 or 2 further substituents as for heteroaryl^{a}.

When B is 7-azaindole, B can be substituted with chloro, and optionally substituted with 1 or 2 further substituents as for heteroaryl^{a}.

When B is 7-azaindole, B can be selected from

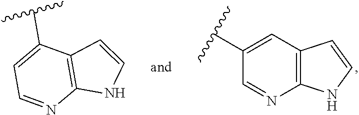 [SMILES: *c1ccnc2[nH]ccc12 ; *c1cnc2[nH]ccc2c1]

substituted with chloro, and optionally substituted with 1 or 2 further substituents as for heteroaryl^{a}.

When B is 7-azaindole, B can be

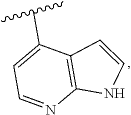 [SMILES: *c1ccnc2[nH]ccc12]

substituted with chloro, and optionally substituted with 1 or 2 further substituents as for heteroaryl^{a}.

When B is azaindole, B can be selected from

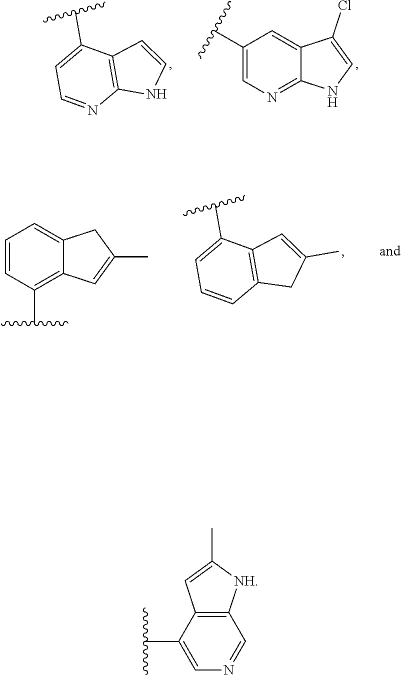 [SMILES: *c1ccnc2[nH]ccc12 ; *c1cnc2[nH]cc(Cl)c2c1 ; *c1cccc2c1C=C(C)C2 ; *c1cccc2c1C=C(C)C2 ; *c1cncc2[nH]c(C)cc12]

Preferably when B is 7-azaindole, B can be selected from

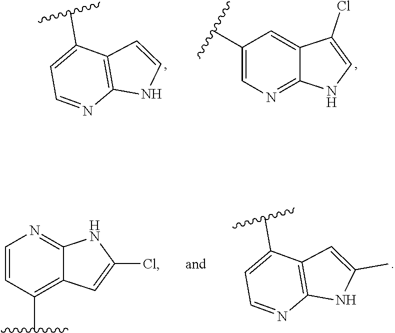 [SMILES: *c1ccnc2[nH]ccc12 ; *c1cnc2[nH]cc(Cl)c2c1 ; *c1ccnc2[nH]c(Cl)cc12 ; "and" ; *c1ccnc2[nH]c(C)cc12]

When B is heteroaryl^{a}, B can selected from

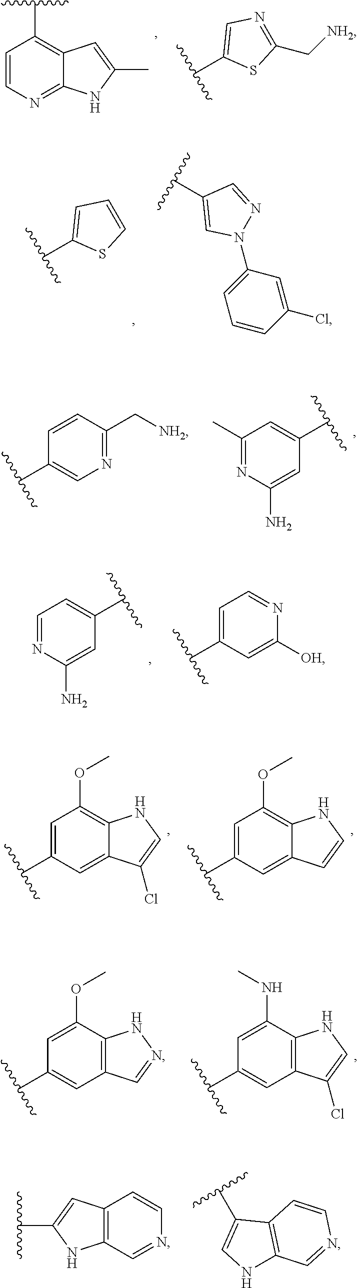

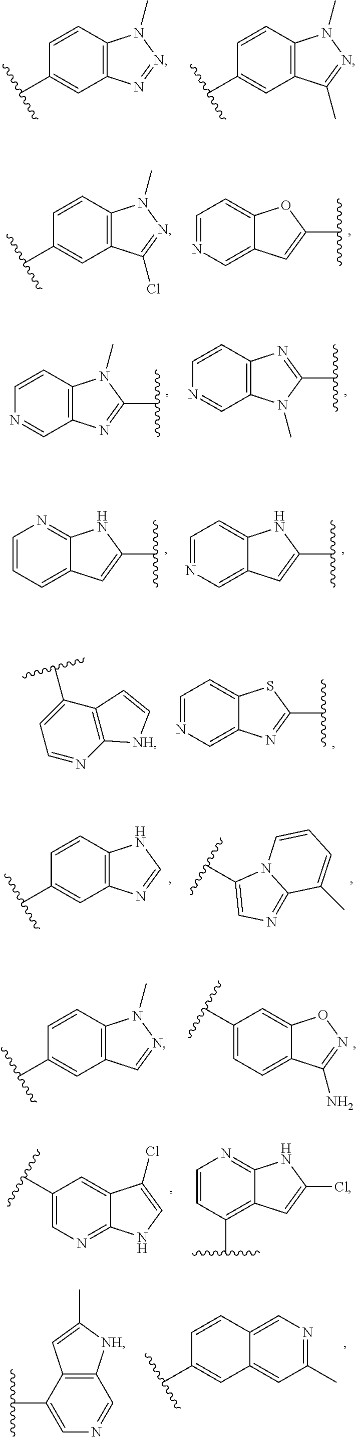

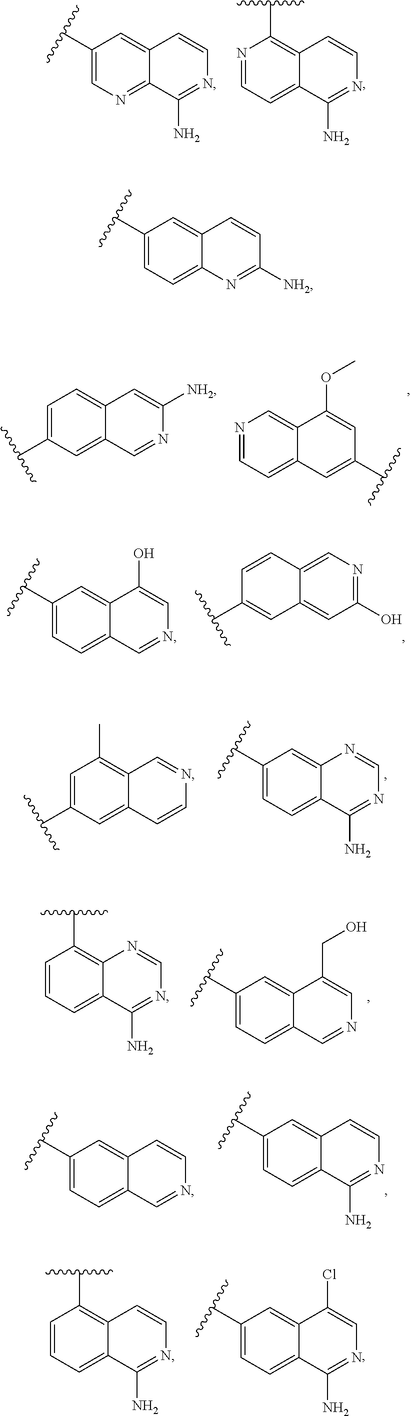

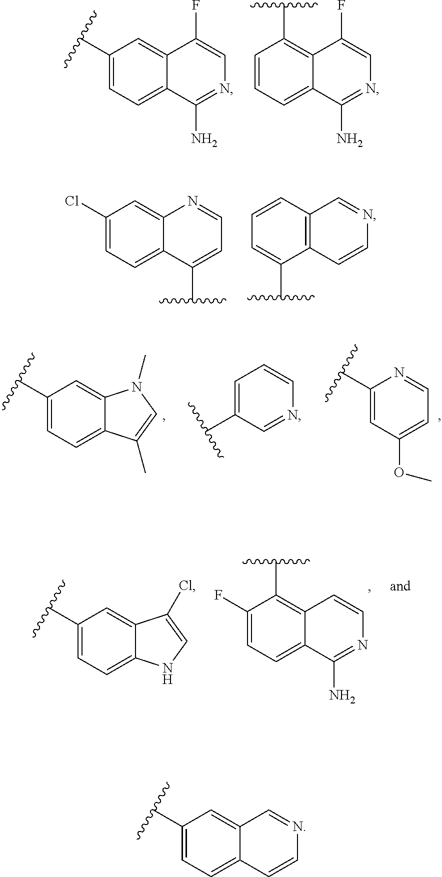

When B is heteroaryl^{a}, B can be selected from

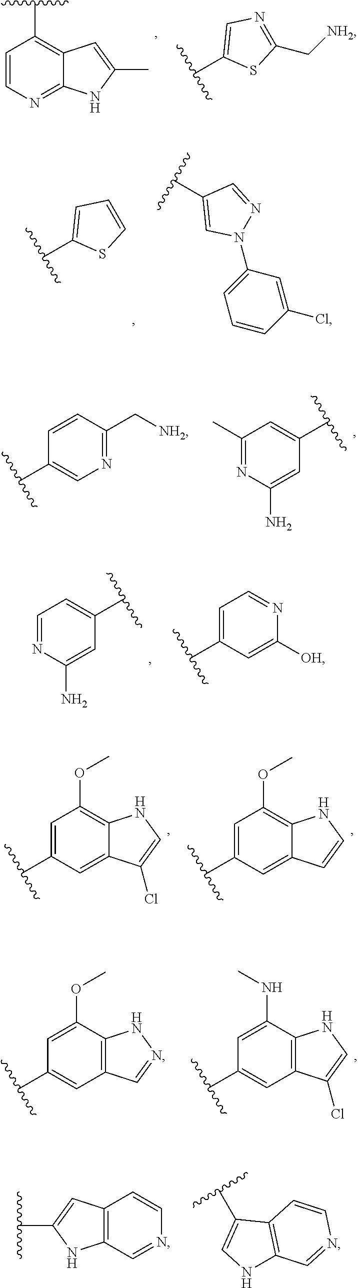

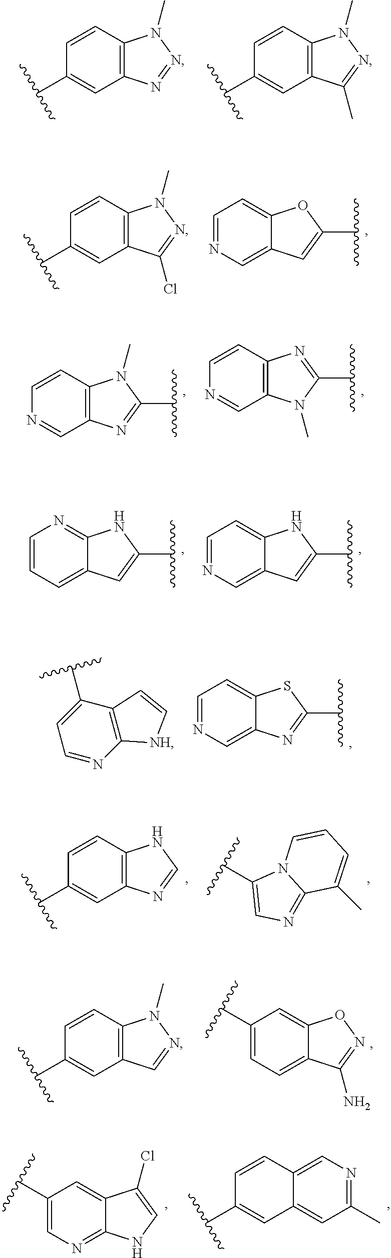

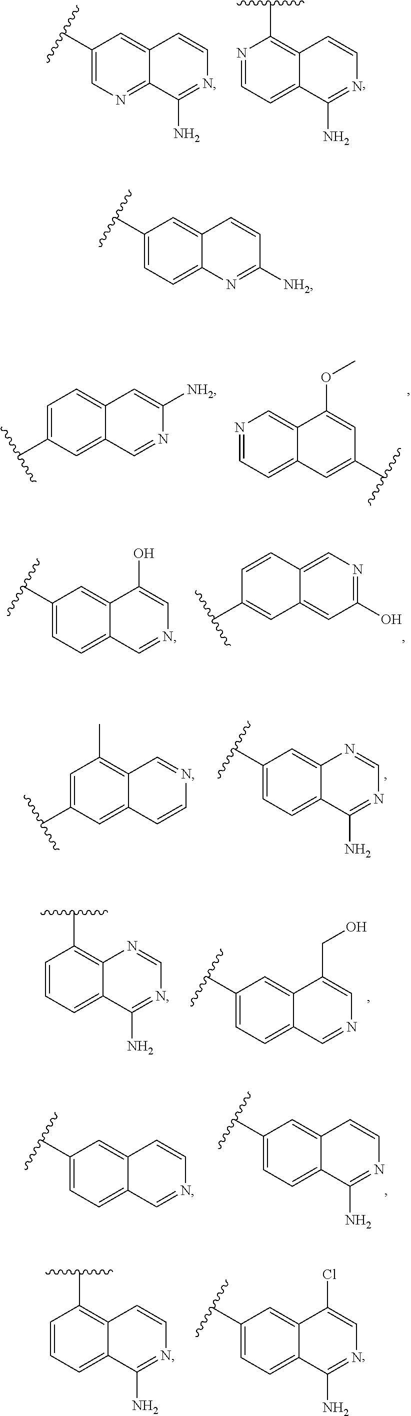

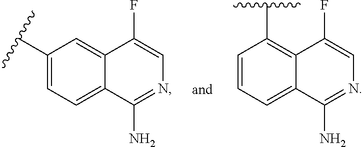 [SMILES: *c1ccc2c(N)ncc(F)c2c1 ; *c1cccc2c(N)ncc(F)c12]

When B is heteroaryl^{a}, B can selected from

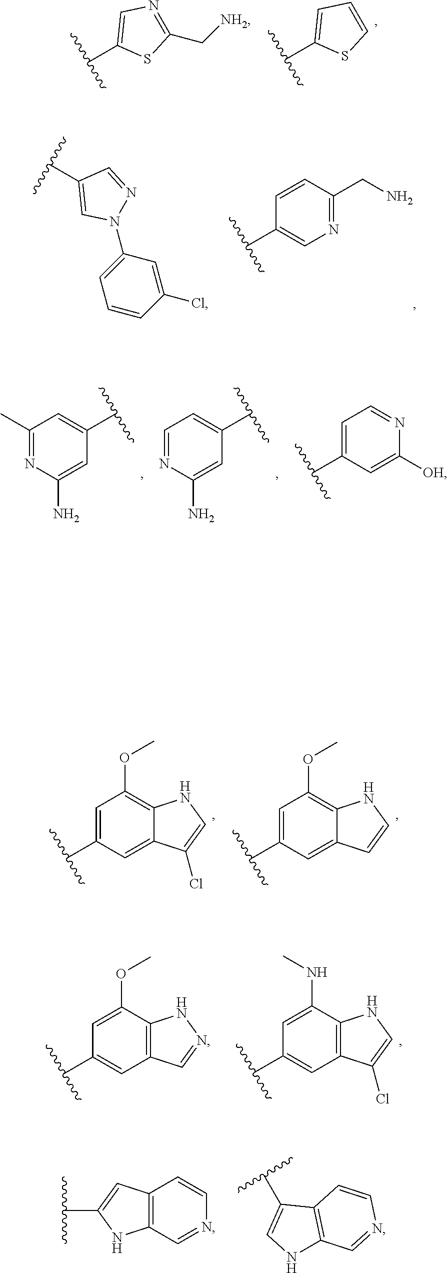

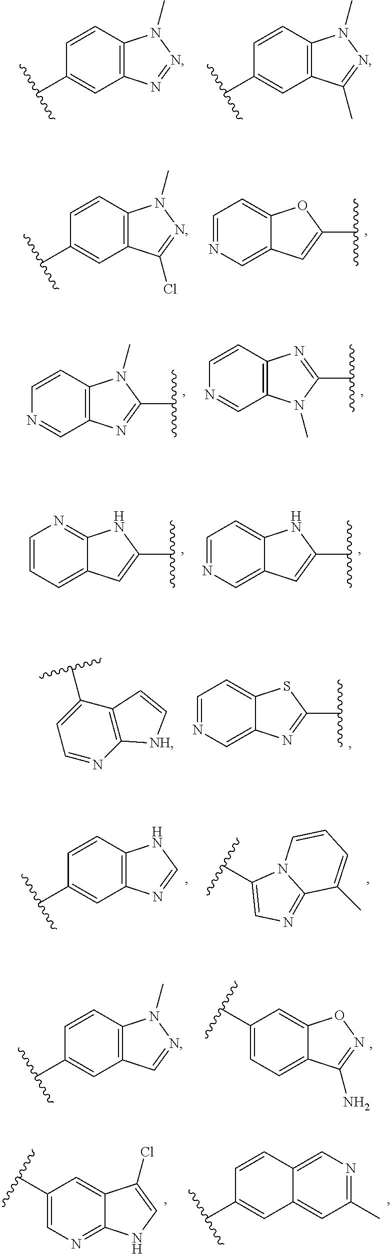

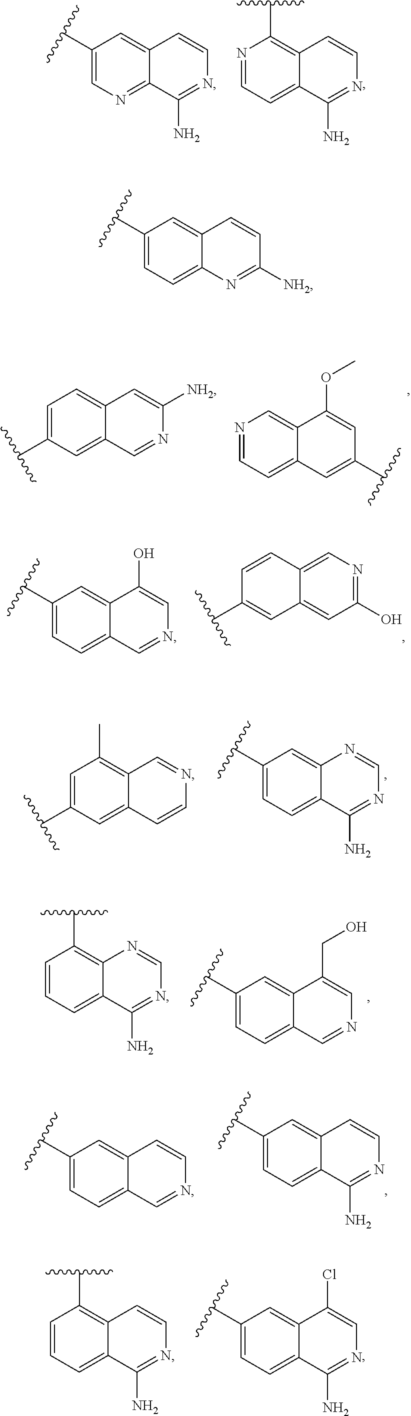

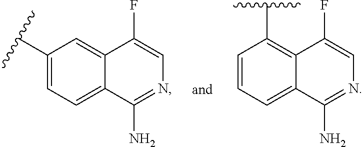 [SMILES: *c1ccc2c(N)ncc(F)c2c1 ; *c1cccc2c(N)ncc(F)c12]

B can be selected from

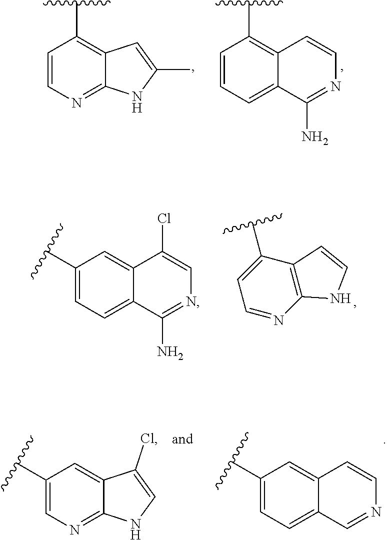

B can be selected from

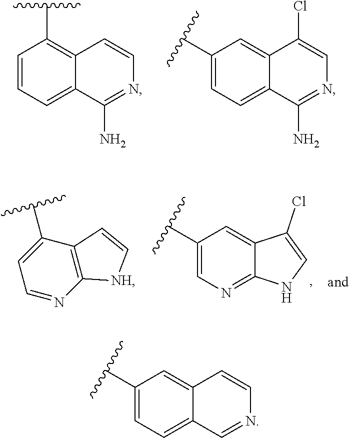

B can be aryl. B can be phenyl or naphthyl, wherein B may be optionally substituted as for aryl. When B is aryl, preferably B is phenyl, wherein B may be optionally substituted as for aryl.

B can be selected from:

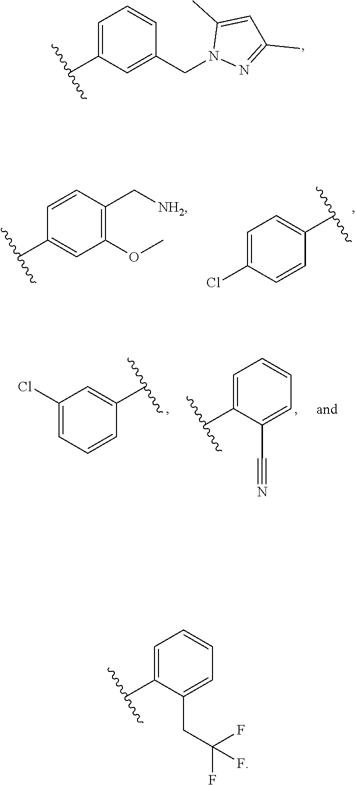 [SMILES: *c1cccc(Cn2nc(C)cc2C)c1 ; *c1ccc(CN)c(OC)c1 ; *c1ccc(Cl)cc1 ; *c1cccc(Cl)c1 ; *c1ccccc1C#N ; *c1ccccc1CC(F)(F)F]

B can be selected from:

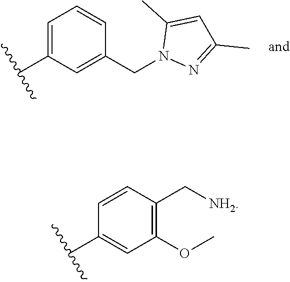 [SMILES: *c1cccc(Cn2nc(C)cc2C)c1 ; *c1ccc(CN)c(OC)c1]

B can be a 5- to 6-membered non-aromatic heterocyclic ring containing one N ring member, which is unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃.

When B is a 5- to 6-membered non-aromatic heterocyclic ring containing one N ring member, which is unsaturated with 1 or 2 double bonds, it is preferably pyridone (e.g. 2-pyridone or 4-pyridone). B can be pyridone which is unsaturated with 2 double bonds, which may be optionally substituted by 1, 2 or 3 substituents independently selected from alkyl, alkoxy, aryl^{b}, OH, OCF₃, halo, oxo, CN, and CF₃. B can be pyridone which is unsaturated with 2 double bonds, substituted by two alkyl groups.

B can be:

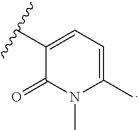 [SMILES: *c1ccc(C)n(C)c1=O]

B can be a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring may be optionally substituted with 1, 2, or 3 substituted by up to three substituents independently selected from alkyl, alkoxy, OH, OCF₃, halo, oxo, CN, and CF₃, wherein the 6,5-bicyclic ring may be attached via the 6- or 5-membered ring.

B can be selected from:

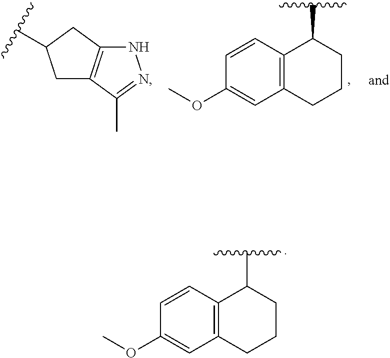

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is N or NH; and B is heteroaryl^{a}. More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is N or NH; and B is isoquinolinyl, optionally substituted as for heteroaryl^{a}.

Yet more preferably, when not absent, -V-Z- is selected from —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is N or NH; and B is isoquinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is N or NH; and B is isoquinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is N or NH; and B is heteroaryl^{a}. More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is N or NH; and B is isoquinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is N or NH; and B is isoquinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is N or NH; and B is isoquinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is N or NH; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is N or NH; and B is isoquinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is N or NH; and B is isoquinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is N or NH; and B is isoquinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is isoquinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is isoquinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is isoquinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is isoquinolinyl, optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is isoquinolinyl, substituted with NH₂, and optionally substituted with 1 or 2 further substituents as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is isoquinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

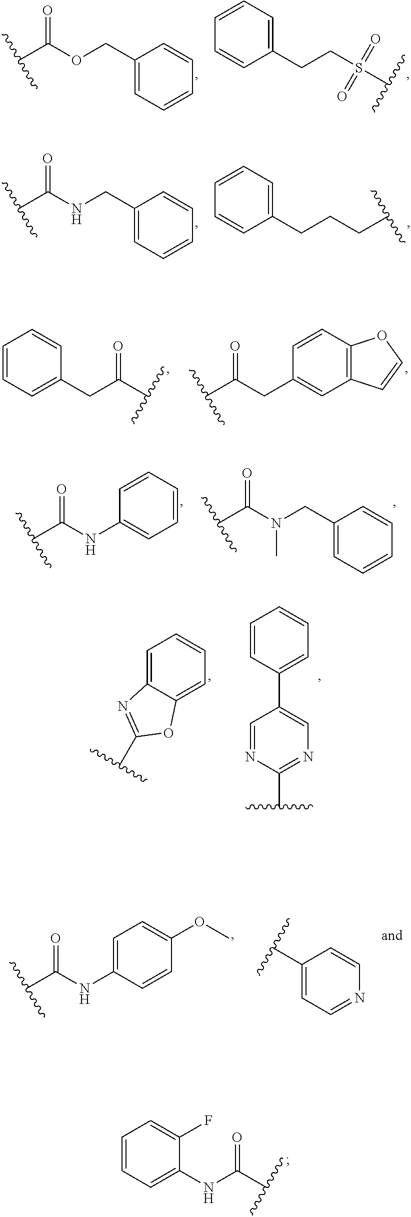

X is CR1R2; R1 is H; R2 is H; Y is NH; and B is heteroaryl^{a}.

More preferably, -V-Z- is selected from —CH₂—, —O—CH₂—, —CH₂—O— and —CH₂—CH₂—O—; X is CR1R2; R1 is H; R2 is H; Y is NH; and B is isoquinolinyl, optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

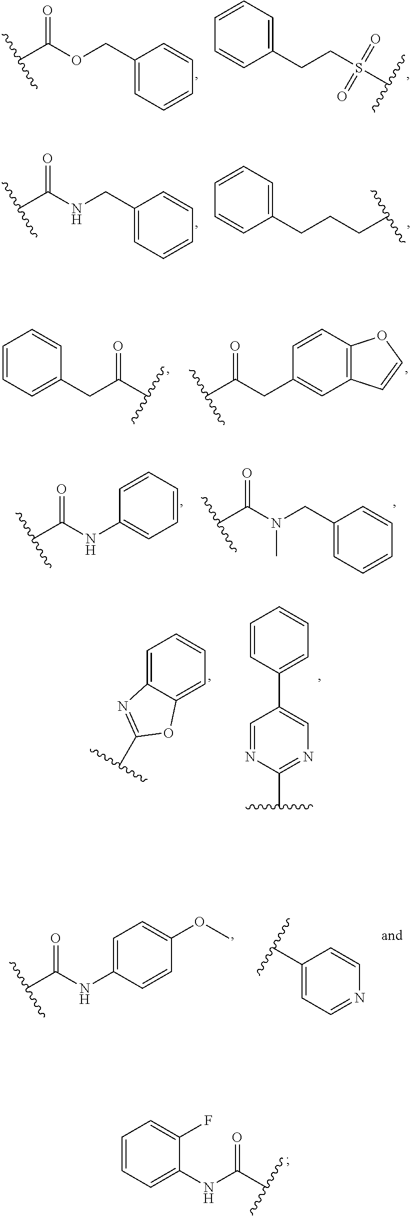

X is CR1R2; R1 is H; R2 is H; Y is NH; and B is isoquinolinyl, substituted with NH₂, and optionally substituted with 1 or 2 further substituents as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:

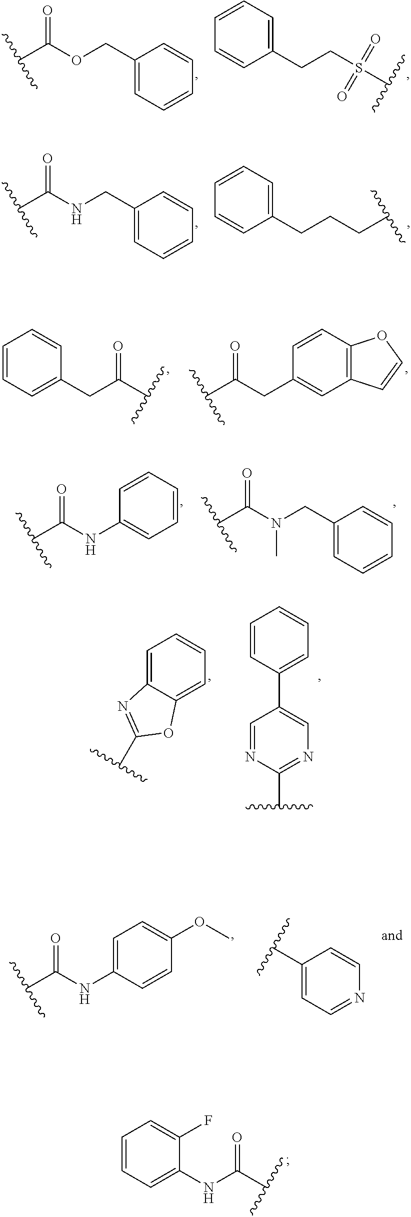

X is CR1R2; R1 is H; R2 is H; Y is NH; and B is isoquinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O— and —CH₂—CH₂—O—; X is CR1R2; R1 is H; R2 is H; Y is NH; and B is heteroaryl^{a}.

More preferably, -V-Z- is selected from —CH₂—, —O—CH₂—, —CH₂—O— and —CH₂—CH₂—O—; X is CR1R2; R1 is H; R2 is H; Y is NH; and B is isoquinolinyl, optionally substituted as for heteroaryl^{a}.

Yet more preferably, -V-Z- is selected from —CH₂—, —O—CH₂—, —CH₂—O— and —CH₂—CH₂—O—; X is CR1R2; R1 is H; R2 is H; Y is NH; and B is isoquinolinyl, substituted with NH₂, and optionally substituted with 1 or 2 further substituents as for heteroaryl^{a}.

More specifically, -V-Z- is selected from —CH₂—, —O—CH₂—, —CH₂—O— and —CH₂—CH₂—O—; X is CR1R2; R1 is H; R2 is H; Y is NH; and B is isoquinolinyl, substituted with NH₂ and halo (e.g. chloro), and optionally 1 further substituent as for heteroaryl^{a}.

Preferably, X is CR1R2; R1 is H; R2 is H; Y is NH; and B is heteroaryl^{a}. More preferably, X is CR1R2; R1 is H; R2 is H; Y is NH; and B is isoquinolinyl, optionally substituted as for heteroaryl^{a}. Yet more preferably, X is CR1R2; R1 is H; R2 is H; Y is NH; and B is isoquinolinyl, substituted with NH₂, and optionally substituted with 1 or 2 further substituents as for heteroaryl^{a}. More specifically, X is CR1R2; R1 is H; R2 is H; Y is NH; and B is isoquinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is quinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is quinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is quinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is quinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is quinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is quinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is quinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is quinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is quinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is quinolinyl, optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is quinolinyl, substituted with NH₂, and optionally substituted with 1 or 2 further substituents as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is quinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

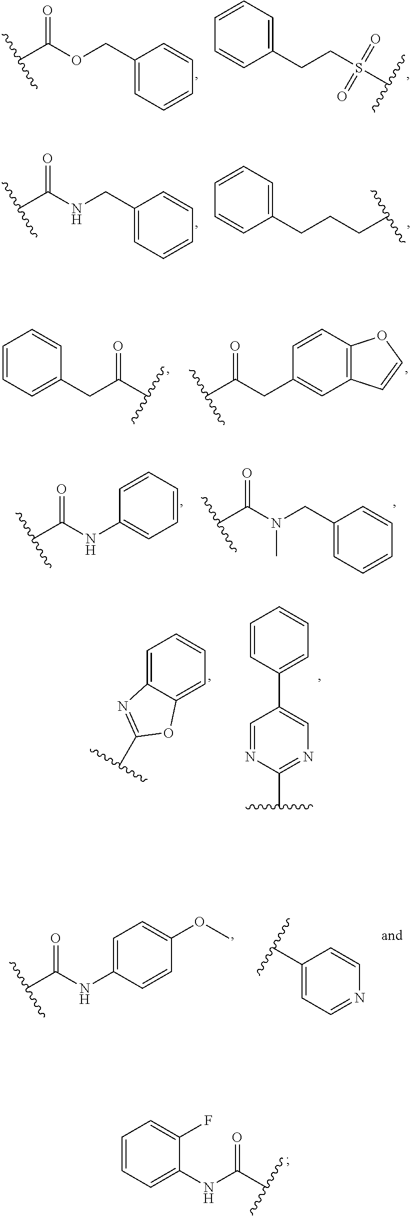

X is CR1R2; R1 is H; R2 is H; Y is NH; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

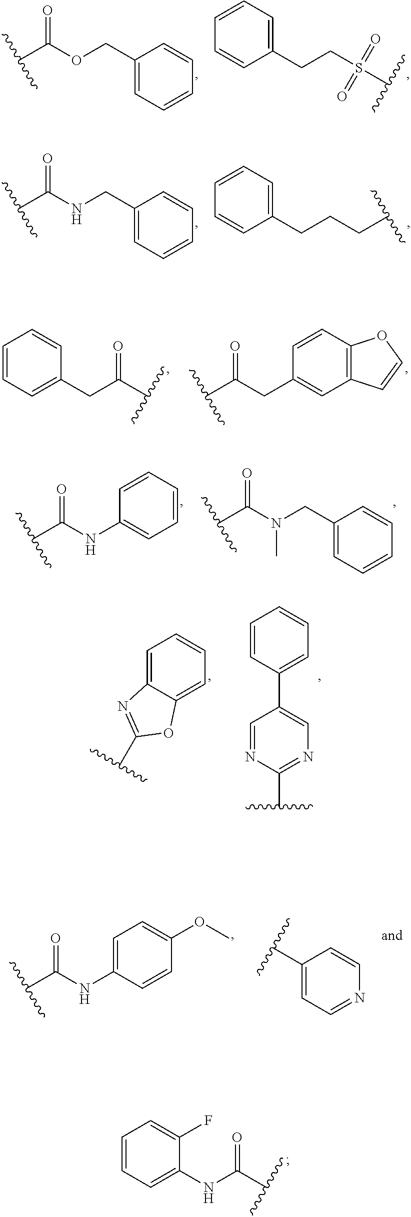

X is CR1R2; R1 is H; R2 is H; Y is NH; and B is quinolinyl, optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

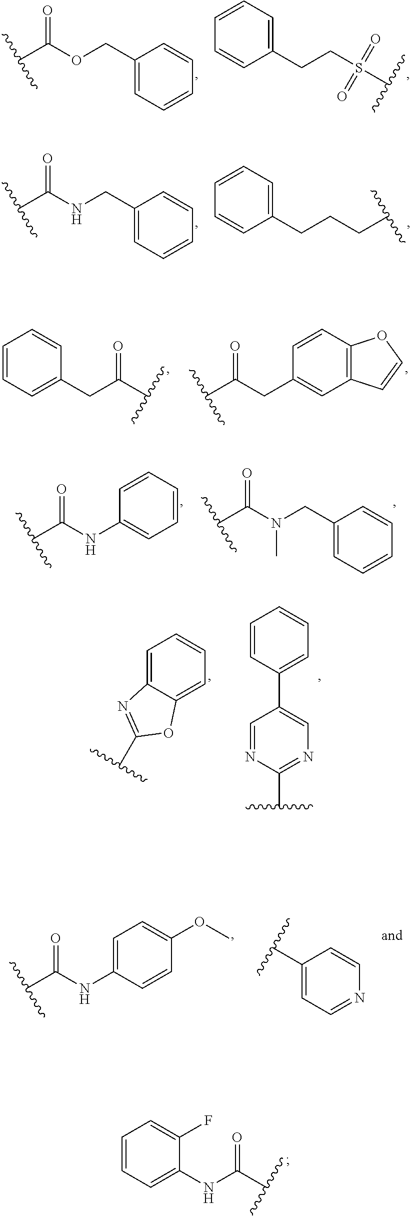

X is CR1R2; R1 is H; R2 is H; Y is NH; and B is quinolinyl, substituted with NH₂, and optionally substituted with 1 or 2 further substituents as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:

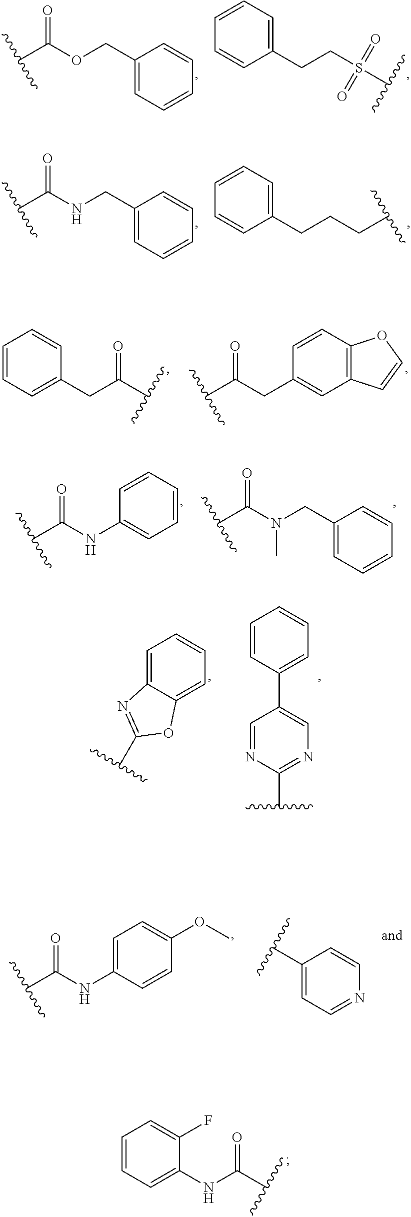

X is CR1R2; R1 is H; R2 is H; Y is NH; and B is quinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O— and —CH₂—CH₂—O—; X is CR1R2; R1 is H; R2 is H; Y is NH; and B is heteroaryl^{a}.

More preferably, -V-Z- is selected from —CH₂—, —O—CH₂—, —CH₂—O— and —CH₂—CH₂—O—; X is CR1R2; R1 is H; R2 is H; Y is NH; and B is quinolinyl, optionally substituted as for heteroaryl^{a}.

Yet more preferably, -V-Z- is selected from —CH₂—, —O—CH₂—, —CH₂—O— and —CH₂—CH₂—O—; X is CR1R2; R1 is H; R2 is H; Y is NH; and B is quinolinyl, substituted with NH₂, and optionally substituted with 1 or 2 further substituents as for heteroaryl^{a}.

More specifically, -V-Z- is selected from —CH₂—, —O—CH₂—, —CH₂—O— and —CH₂—CH₂—O—; X is CR1R2; R1 is H; R2 is H; Y is NH; and B is quinolinyl, substituted with NH₂ and halo (e.g. chloro), and optionally 1 further substituent as for heteroaryl^{a}.

Preferably, X is CR1R2; R1 is H; R2 is H; Y is NH; and B is heteroaryl^{a}. More preferably, X is CR1R2; R1 is H; R2 is H; Y is NH; and B is quinolinyl, optionally substituted as for heteroaryl^{a}. Yet more preferably, X is CR1R2; R1 is H; R2 is H; Y is NH; and B is quinolinyl, substituted with NH₂, and optionally substituted with 1 or 2 further substituents as for heteroaryl^{a}. More specifically, X is CR1R2; R1 is H; R2 is H; Y is NH; and B is quinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is azaindole (particularly 7-azaindole) substituted with alkyl, and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is 7-azaindole), substituted with NH₂, and optionally substituted with 1 or 2 further substituents as for heteroaryl^{b}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is azaindole (particularly 7-azaindole) substituted with methyl, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is azaindole (particularly 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is azaindole (particularly 7-azaindole) substituted with alkyl, and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is azaindole (particularly 7-azaindole), substituted with NH₂, and optionally substituted with 1 or 2 further substituents as for heteroaryl^{b}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—; X is CR1R2; R1 is H; R2 is H; Y is NH or N; and B is azaindole (particularly 7-azaindole) substituted with methyl, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:

(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is azaindole (particularly 7-azaindole) substituted with alkyl, and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is azaindole (particularly 7-azaindole), substituted with NH₂, and optionally substituted with 1 or 2 further substituents as for heteroaryl^{b}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is azaindole (particularly 7-azaindole) substituted with methyl, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is azaindole (particularly 7-azaindole), substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is azaindole (particularly 7-azaindole), optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is azaindole (particularly 7-azaindole) substituted with alkyl, and optionally substituted with 1 or 2 further substituents as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is azaindole (particularly 7-azaindole), substituted with NH₂, and optionally substituted with 1 or 2 further substituents as for heteroaryl^{b}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is azaindole (particularly 7-azaindole) substituted with methyl, and optionally substituted with 1 or 2 further substituents as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is azaindole (particularly 7-azaindole), substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

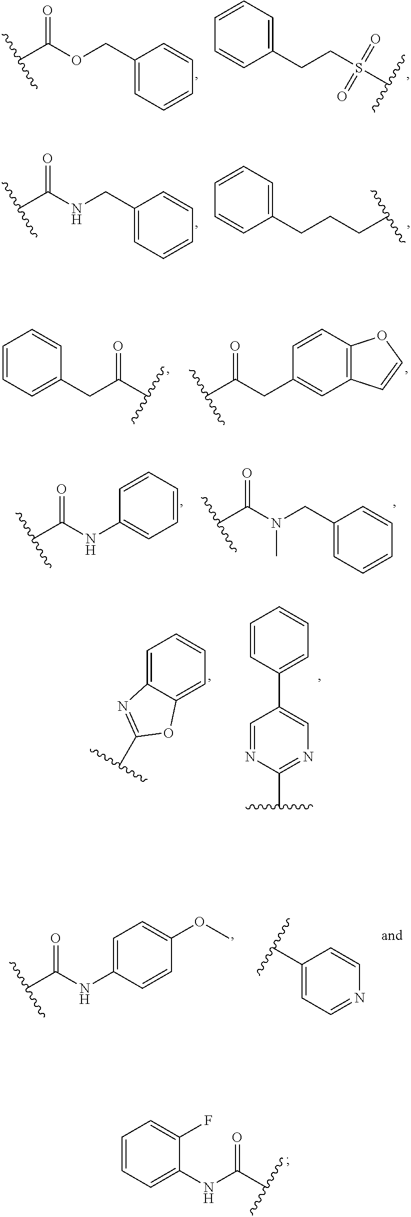

X is CR1R2; R1 is H; R2 is H; Y is NH; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

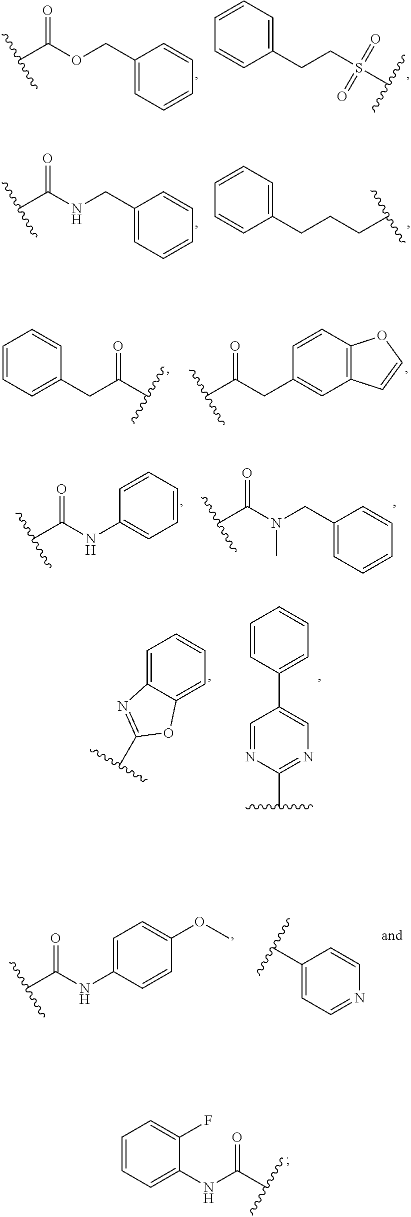

X is CR1R2; R1 is H; R2 is H; Y is NH; and B is azaindole (particularly 7-azaindole), optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

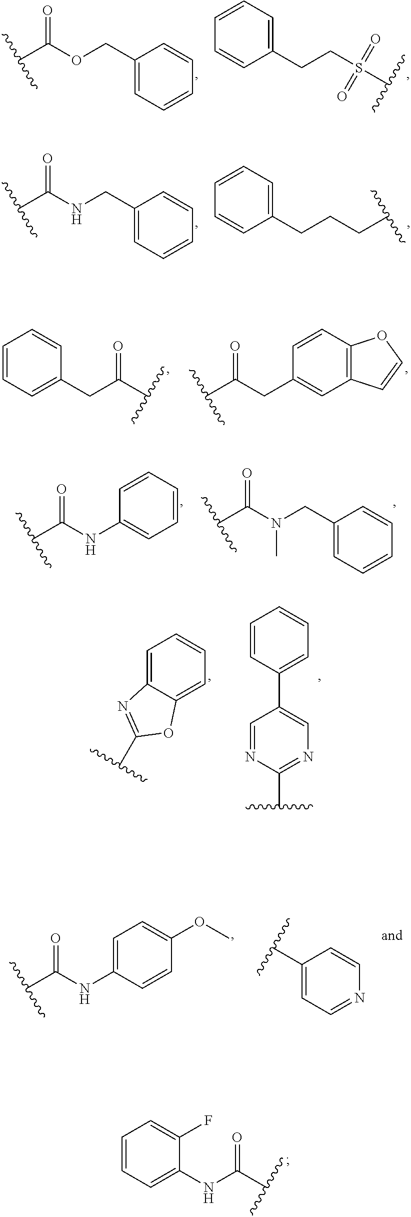

X is CR1R2; R1 is H; R2 is H; Y is NH; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

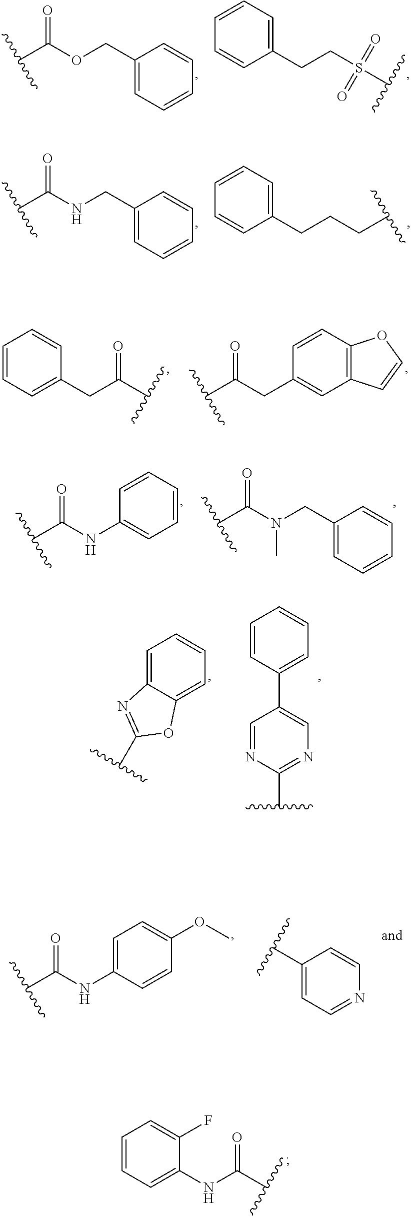

X is CR1R2; R1 is H; R2 is H; Y is NH; and B is azaindole (particularly 7-azaindole) substituted with alkyl, and optionally substituted with 1 or 2 further substituents as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

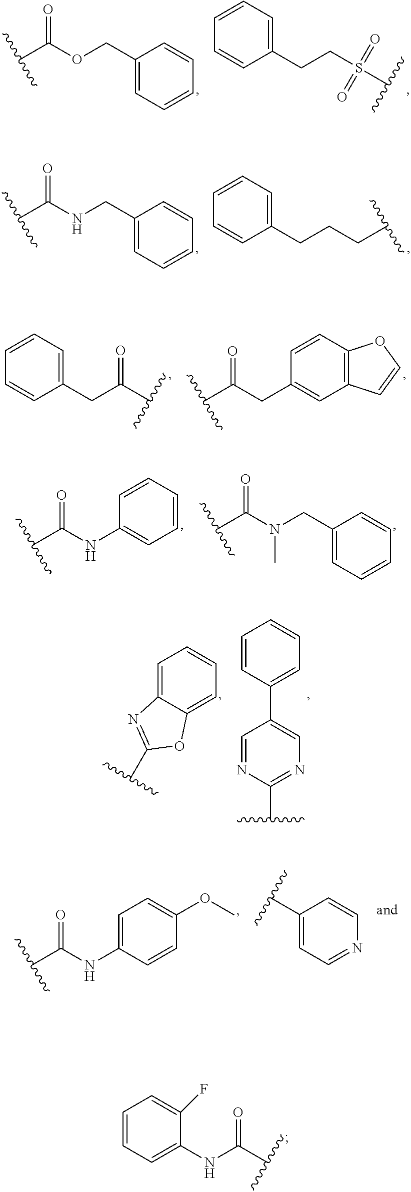

X is CR1R2; R1 is H; R2 is H; Y is NH; and B is azaindole (particularly 7-azaindole), substituted with NH₂, and optionally substituted with 1 or 2 further substituents as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

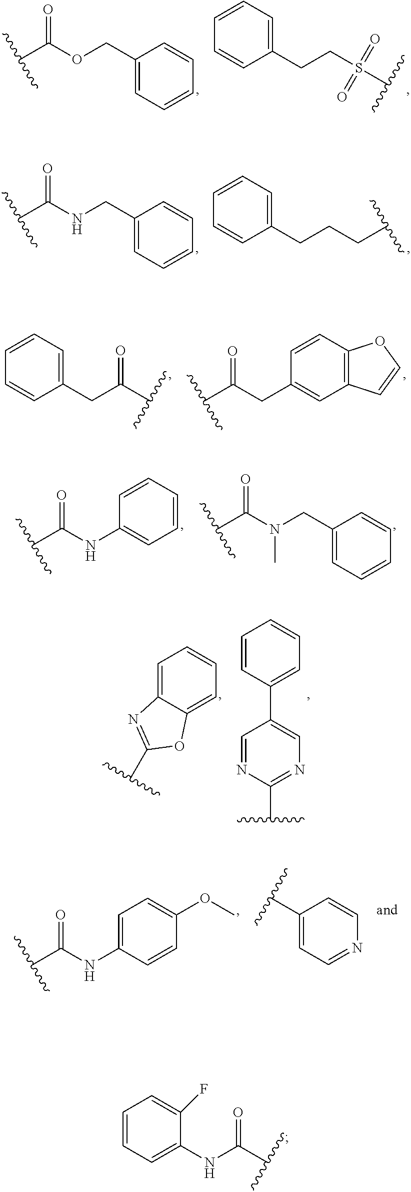

X is CR1R2; R1 is H; R2 is H; Y is NH; and B is azaindole (particularly 7-azaindole) substituted with methyl, and optionally substituted with 1 or 2 further substituents as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:

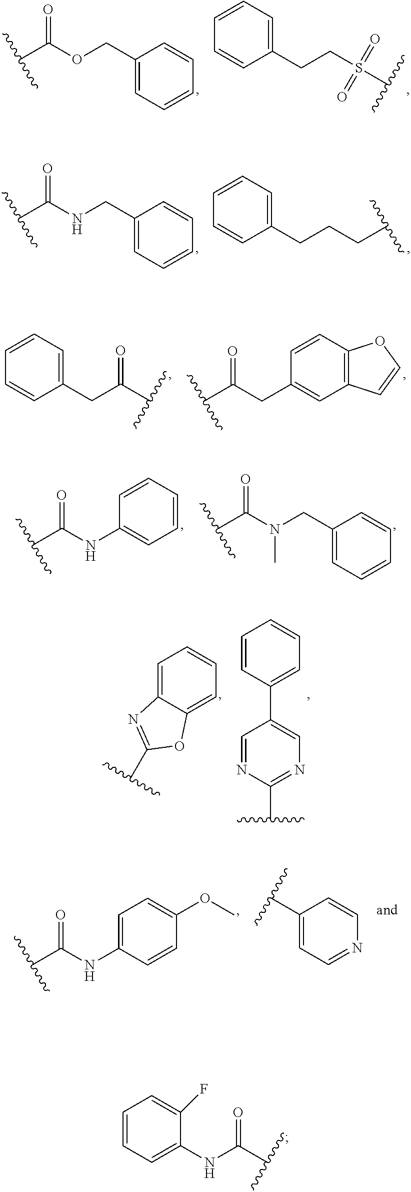

X is CR1R2; R1 is H; R2 is H; Y is NH; and B is azaindole (particularly 7-azaindole), substituted with NH₂ and halo (e.g. chloro), and optionally 1 further substituent as for heteroaryl^{a}.

Preferably, -V-Z- is selected from —CH₂—, —O—CH₂—, —CH₂—O— and —CH₂—CH₂—O—; X is CR1R2; R1 is H; R2 is H; Y is NH; and B is heteroaryl^{a}.

More preferably, -V-Z- is selected from —CH₂—, —O—CH₂—, —CH₂—O— and —CH₂—CH₂—O—; X is CR1R2; R1 is H; R2 is H; Y is NH; and B is azaindole (particularly 7-azaindole), optionally substituted as for heteroaryl^{a}.

Yet more preferably, -V-Z- is selected from —CH₂—, —O—CH₂—, —CH₂—O— and —CH₂—CH₂—O—; X is CR1R2; R1 is H; R2 is H; Y is NH; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O— and —CH₂—CH₂—O—; X is CR1R2; R1 is H; R2 is H; Y is NH; and B is azaindole (particularly 7-azaindole) substituted with alkyl, and optionally substituted with 1 or 2 further substituents as for heteroaryl^{a}.

Alternatively, -V-Z- is selected from —CH₂—, —O—CH₂—, —CH₂—O— and —CH₂—CH₂—O—; X is CR1R2; R1 is H; R2 is H; Y is NH; and B is azaindole (particularly 7-azaindole), substituted with NH₂, and optionally substituted with 1 or 2 further substituents as for heteroaryl^{b}.

Preferably, -V-Z- is selected from —CH₂—, —O—CH₂—, —CH₂—O— and —CH₂—CH₂—O—; X is CR1R2; R1 is H; R2 is H; Y is NH; and B is azaindole (particularly 7-azaindole) substituted with methyl, and optionally substituted with 1 or 2 further substituents as for heteroaryl^{a}.

More specifically, -V-Z- is selected from —CH₂—, —O—CH₂—, —CH₂—O— and —CH₂—CH₂—O—; X is CR1R2; R1 is H; R2 is H; Y is NH; and B is azaindole (particularly 7-azaindole), substituted with NH₂ and halo (e.g. chloro), and optionally 1 further substituent as for heteroaryl^{a}.

Preferably, X is CR1R2; R1 is H; R2 is H; Y is NH; and B is heteroaryl^{a}. More preferably, X is CR1R2; R1 is H; R2 is H; Y is NH; and B is azaindole (particularly 7-azaindole), optionally substituted as for heteroaryl^{a}. Yet more preferably, X is CR1R2; R1 is H; R2 is H; Y is NH; and B is azaindole (particularly 7-azaindole), substituted with halo (e.g. chloro), and optionally substituted with 1 or 2 further substituents as for heteroaryl^{a}. Alternatively, X is CR1R2; R1 is H; R2 is H; Y is NH; and B is azaindole (particularly 7-azaindole) substituted with alkyl, and optionally substituted with 1 or 2 further substituents as for heteroaryl^{a}. Alternatively, X is CR1R2; R1 is H; R2 is H; Y is NH; and B is azaindole (particularly 7-azaindole), substituted with NH₂, and optionally substituted with 1 or 2 further substituents as for heteroaryl^{b}. Preferably, X is CR1R2; R1 is H; R2 is H; Y is NH; and B is azaindole (particularly 7-azaindole) substituted with methyl, and optionally substituted with 1 or 2 further substituents as for heteroaryl^{a}. More specifically, X is CR1R2; R1 is H; R2 is H; Y is NH; and B is azaindole (particularly 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
X is CR1R2; R1 is H; R2 is H; Y is NH or N; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
X is CR1R2; R1 is H; R2 is H; Y is NH or N; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
X is CR1R2; R1 is H; R2 is H; Y is NH or N; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
X is CR1R2; R1 is H; R2 is H; Y is NH or N; and B is azaindole (particularly 7-azaindole), substituted with NH₂, and optionally substituted with 1 or 2 further substituents as for heteroaryl^{b}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
X is CR1R2; R1 is H; R2 is H; Y is NH or N; and B is azaindole (particularly 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
X is CR1R2; R1 is H; R2 is H; Y is NH or N; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
X is CR1R2; R1 is H; R2 is H; Y is NH or N; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
X is CR1R2; R1 is H; R2 is H; Y is NH or N; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
X is CR1R2; R1 is H; R2 is H; Y is NH or N; and B is azaindole (particularly 7-azaindole), substituted with NH₂, and optionally substituted with 1 or 2 further substituents as for heteroaryl^{b}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
X is CR1R2; R1 is H; R2 is H; Y is NH or N; and B is azaindole (particularly 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is azaindole (particularly 7-azaindole), substituted with NH₂, and optionally substituted with 1 or 2 further substituents as for heteroaryl^{b}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is NH; and B is azaindole (particularly 7-azaindole), substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is azaindole (particularly 7-azaindole), optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is azaindole (particularly 7-azaindole), substituted with NH₂, and optionally substituted with 1 or 2 further substituents as for heteroaryl^{b}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is NH; and B is azaindole (particularly 7-azaindole), substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

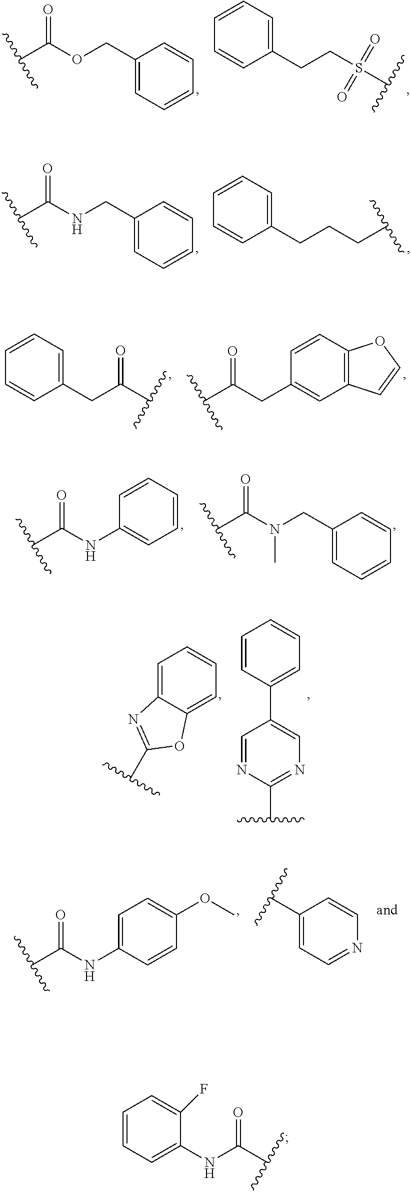

X is CR1R2; R1 is H; R2 is H; Y is NH; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂, wherein R18 is selected from:

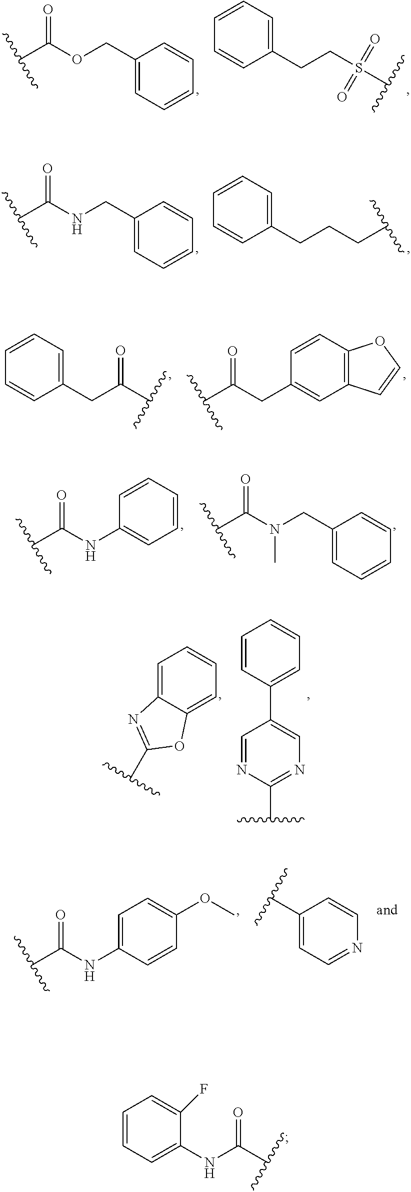

X is CR1R2; R1 is H; R2 is H; Y is NH; and B is azaindole (particularly 7-azaindole), optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

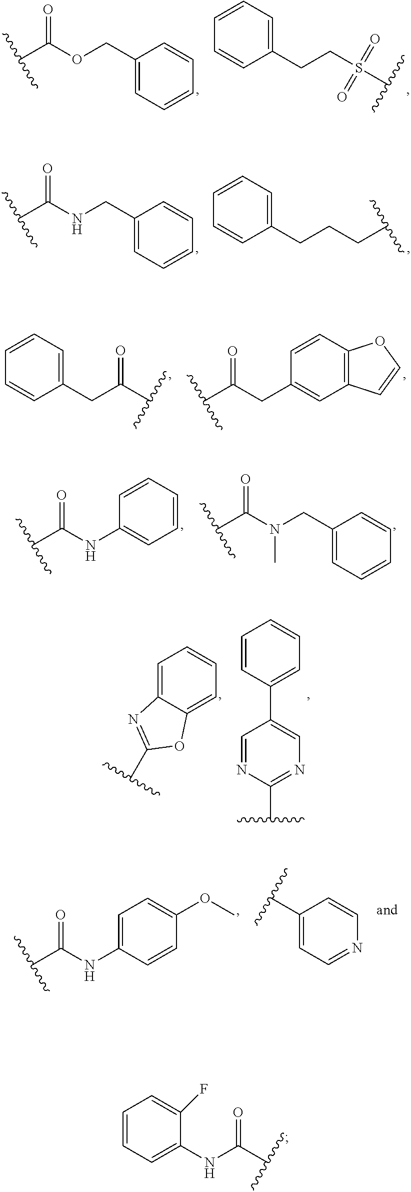

X is CR1R2; R1 is H; R2 is H; Y is NH; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

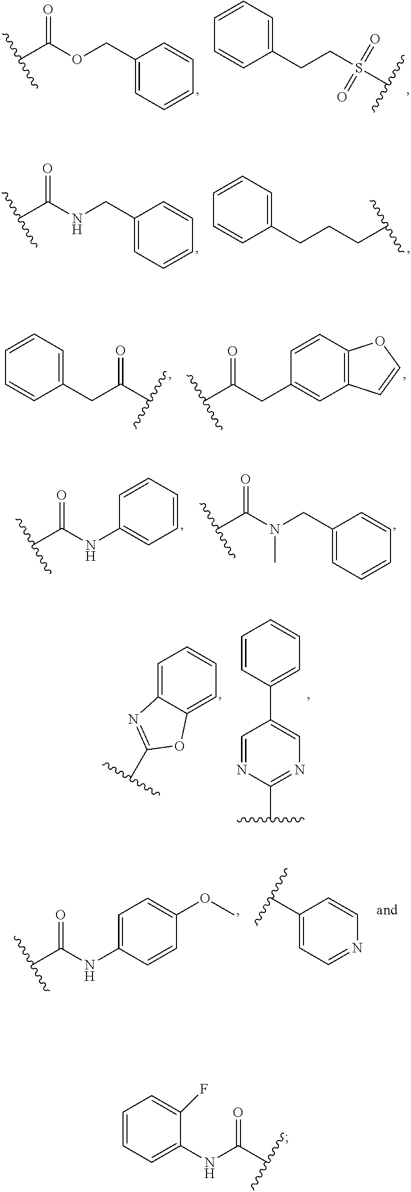

X is CR1R2; R1 is H; R2 is H; Y is NH; and B is azaindole (particularly 7-azaindole), substituted with NH₂, and optionally substituted with 1 or 2 further substituents as for heteroaryl^{b}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:

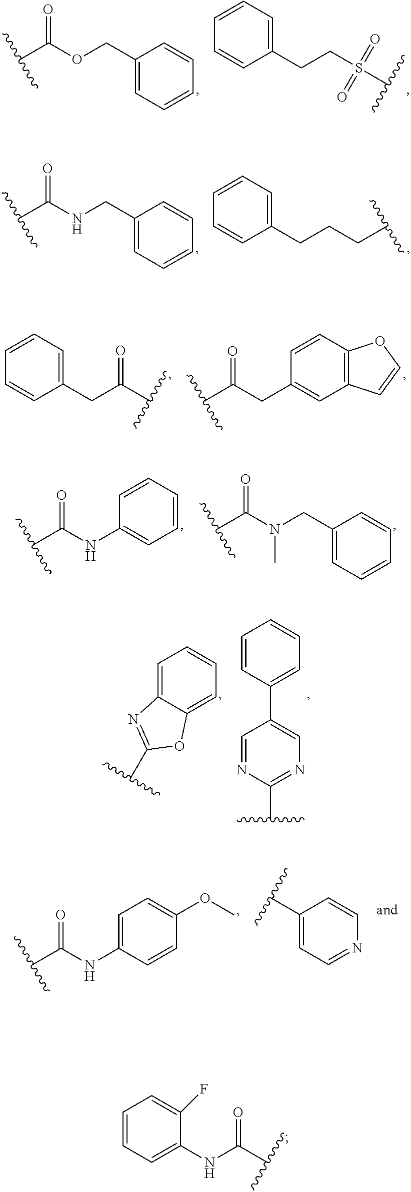

X is CR1R2; R1 is H; R2 is H; Y is NH; and B is azaindole (particularly 7-azaindole), substituted with NH₂ and halo (e.g. chloro), and optionally 1 further substituent as for heteroaryl^{a}.

Preferably, -V-Z- is selected from —CH₂—, —O—CH₂—, —CH₂—O— and —CH₂—CH₂—O—; X is CR1R2; R1 is H; R2 is H; Y is NH; and B is heteroaryl^{a}.

More preferably, -V-Z- is selected from —CH₂—, —O—CH₂—, —CH₂—O— and —CH₂—CH₂—O—; X is CR1R2; R1 is H; R2 is H; Y is NH; and B is azaindole (particularly 7-azaindole), optionally substituted as for heteroaryl^{a}.

Yet more preferably, -V-Z- is selected from —CH₂—, —O—CH₂—, —CH₂—O— and —CH₂—CH₂—O—; X is CR1R2; R1 is H; R2 is H; Y is NH; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O— and —CH₂—CH₂—O—; X is CR1R2; R1 is H; R2 is H; Y is NH; and B is azaindole (particularly 7-azaindole), substituted with NH₂, and optionally substituted with 1 or 2 further substituents as for heteroaryl^{b}.

More specifically, -V-Z- is selected from —CH₂—, —O—CH₂—, —CH₂—O— and —CH₂—CH₂—O—; X is CR1R2; R1 is H; R2 is H; Y is NH; and B is azaindole (particularly 7-azaindole), substituted with NH₂ and halo (e.g. chloro), and optionally 1 further substituent as for heteroaryl^{a}.

Preferably, X is CR1R2; R1 is H; R2 is H; Y is NH; and B is heteroaryl^{a}. More preferably, X is CR1R2; R1 is H; R2 is H; Y is NH; and B is azaindole (particularly 7-azaindole), optionally substituted as for heteroaryl^{a}. Yet more preferably, X is CR1R2; R1 is H; R2 is H; Y is NH; and B is azaindole (particularly 7-azaindole), substituted with halo (e.g. chloro), and optionally substituted with 1 or 2 further substituents as for heteroaryl^{a}. Alternatively, X is CR1R2; R1 is H; R2 is H; Y is NH; and B is azaindole (particularly 7-azaindole), substituted with NH₂, and optionally substituted with 1 or 2 further substituents as for heteroaryl^{b}. More specifically, X is CR1R2; R1 is H; R2 is H; Y is NH; and B is azaindole (particularly 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is isoquinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is isoquinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is isoquinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is isoquinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is isoquinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is isoquinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is isoquinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is isoquinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is isoquinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is isoquinolinyl, optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is isoquinolinyl, substituted with NH₂, and optionally substituted with 1 or 2 further substituents as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is isoquinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O— and —CH₂—CH₂—O—; X is CR1R2; R1 is H; R2 is H; Y is O; and B is heteroaryl^{a}.

More preferably, -V-Z- is selected from —CH₂—, —O—CH₂—, —CH₂—O— and —CH₂—CH₂—O—; X is CR1R2; R1 is H; R2 is H; Y is O; and B is isoquinolinyl, optionally substituted as for heteroaryl^{a}.

Yet more preferably, -V-Z- is selected from —CH₂—, —O—CH₂—, —CH₂—O— and —CH₂—CH₂—O—; X is CR1R2; R1 is H; R2 is H; Y is O; and B is isoquinolinyl, substituted with NH₂, and optionally substituted with 1 or 2 further substituents as for heteroaryl^{a}.

More specifically, -V-Z- is selected from —CH₂—, —O—CH₂—, —CH₂—O— and —CH₂—CH₂—O—; X is CR1R2; R1 is H; R2 is H; Y is O; and B is isoquinolinyl, substituted with NH₂ and halo (e.g. chloro), and optionally 1 further substituent as for heteroaryl^{a}.

Preferably, X is CR1R2; R1 is H; R2 is H; Y is O; and B is heteroaryl^{a}. More preferably, X is CR1R2; R1 is H; R2 is H; Y is O; and B is isoquinolinyl, optionally substituted as for heteroaryl^{a}. Yet more preferably, X is CR1R2; R1 is H; R2 is H; Y is O; and B is isoquinolinyl, substituted with NH₂, and optionally substituted with 1 or 2 further substituents as for heteroaryl^{a}. More specifically, X is CR1R2; R1 is H; R2 is H; Y is O; and B is isoquinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is quinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is quinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is quinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is quinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is quinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is quinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is quinolinyl, optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is quinolinyl, substituted with NH₂, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is quinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is quinolinyl, optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is quinolinyl, substituted with NH₂, and optionally substituted with 1 or 2 further substituents as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is quinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

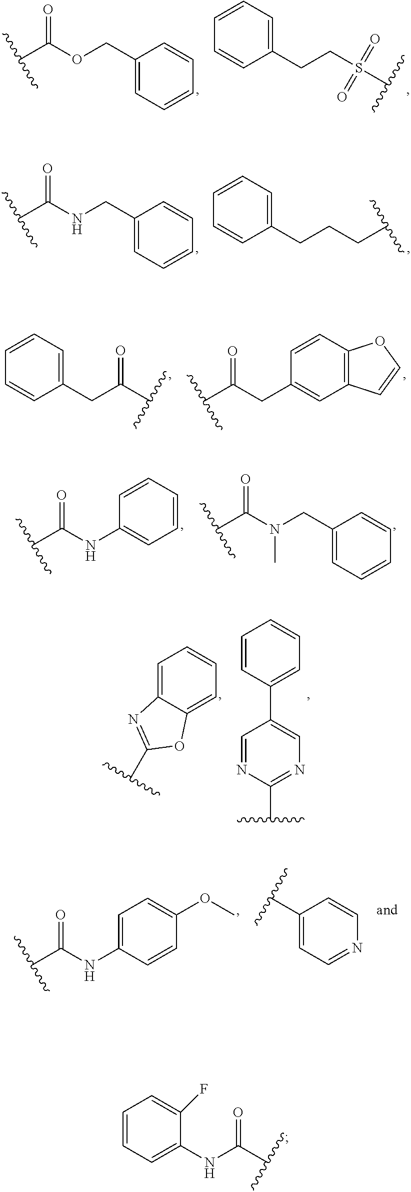

X is CR1R2; R1 is H; R2 is H; Y is O; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

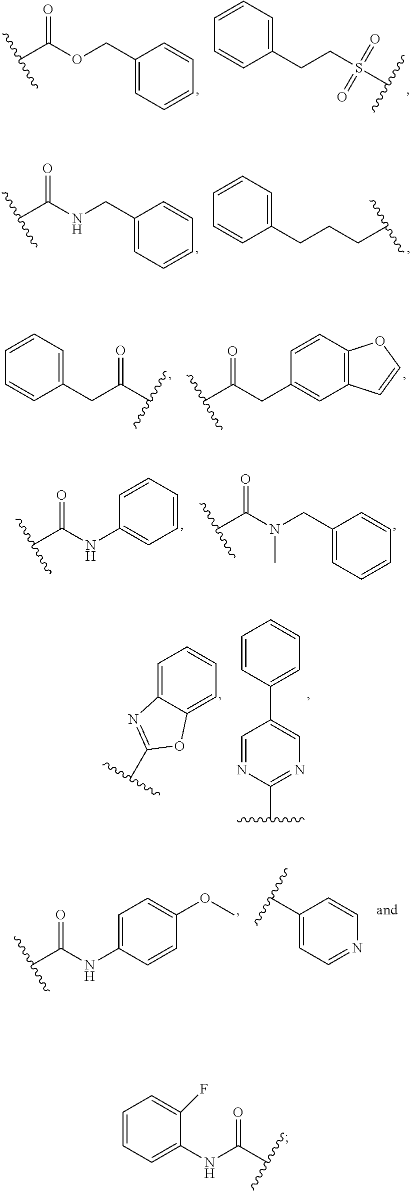

X is CR1R2; R1 is H; R2 is H; Y is O; and B is quinolinyl, optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

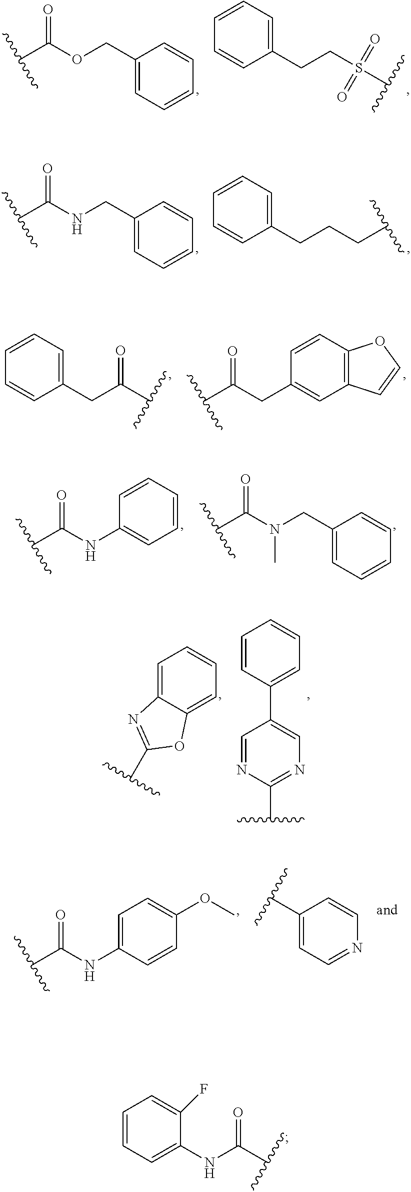

X is CR1R2; R1 is H; R2 is H; Y is O; and B is quinolinyl, substituted with NH₂, and optionally substituted with 1 or 2 further substituents as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:

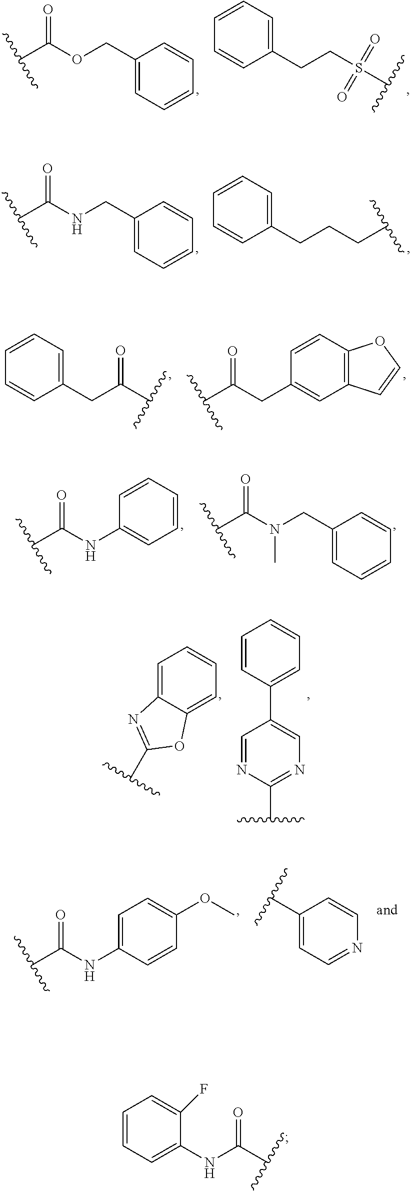

X is CR1R2; R1 is H; R2 is H; Y is O; and B is quinolinyl, substituted with NH₂ and halo (e.g. chloro), and optionally 1 further substituent as for heteroaryl^{a}.

Preferably, -V-Z- is selected from —CH₂—, —O—CH₂—, —CH₂—O— and —CH₂—CH₂—O—; X is CR1R2; R1 is H; R2 is H; Y is O; and B is heteroaryl^{a}.

More preferably, -V-Z- is selected from —CH₂—, —O—CH₂—, —CH₂—O— and —CH₂—CH₂—O—; X is CR1R2; R1 is H; R2 is H; Y is O; and B is quinolinyl, optionally substituted as for heteroaryl^{a}.

Yet more preferably, -V-Z- is selected from —CH₂—, —O—CH₂—, —CH₂—O— and —CH₂—CH₂—O—; X is CR1R2; R1 is H; R2 is H; Y is O; and B is quinolinyl, substituted with NH₂, and optionally substituted with 1 or 2 further substituents as for heteroaryl^{a}.

More specifically, -V-Z- is selected from —CH₂—, —O—CH₂—, —CH₂—O— and —CH₂—CH₂—O—; X is CR1R2; R1 is H; R2 is H; Y is O; and B is quinolinyl, substituted with NH₂ and halo (e.g. chloro), and optionally 1 further substituent as for heteroaryl^{a}.

Preferably, X is CR1R2; R1 is H; R2 is H; Y is O; and B is heteroaryl^{a}. More preferably, X is CR1R2; R1 is H; R2 is H; Y is O; and B is quinolinyl, optionally substituted as for heteroaryl^{a}. Yet more preferably, X is CR1R2; R1 is H; R2 is H; Y is O; and B is quinolinyl, substituted with NH₂, and optionally substituted with 1 or 2 further substituents as for heteroaryl^{a}. More specifically, X is CR1R2; R1 is H; R2 is H; Y is O; and B is quinolinyl, substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a}; CH₂ or —CH₂CH₂—; X is CR1R2; R1 is H; R2 is H; Y is O; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
CH₂ or —CH₂CH₂—; X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with alkyl, and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂, and optionally substituted with 1 or 2 further substituents as for heteroaryl^{b}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with methyl, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
CH₂ or —CH₂CH₂—; X is CR1R2; R1 is H; R2 is H; Y is O; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
CH₂ or —CH₂CH₂—; X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19; wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with alkyl, and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂, and optionally substituted with 1 or 2 further substituents as for heteroaryl^{b}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with methyl, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), substituted with alkyl, and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), substituted with NH₂, and optionally substituted with 1 or 2 further substituents as for heteroaryl^{b}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), substituted with methyl, and optionally substituted with 1 or 2 further substituents as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, -V-Z- is selected from CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is heteroaryl^{a}.

More preferably, -V-Z- is selected from CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is azaindole (particularly 7-azaindole), optionally substituted as for heteroaryl^{a}.

Yet more preferably, -V-Z- is selected from CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, -V-Z- is selected from CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is azaindole (particularly 7-azaindole), substituted with alkyl, and optionally substituted with 1 or 2 further substituents as for heteroaryl^{a}.

Alternatively, -V-Z- is selected from CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂, and optionally substituted with 1 or 2 further substituents as for heteroaryl^{b}.

Preferably, -V-Z- is selected from CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is azaindole (particularly 7-azaindole), substituted with methyl, and optionally substituted with 1 or 2 further substituents as for heteroaryl^{a}.

More specifically, -V-Z- is selected from CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

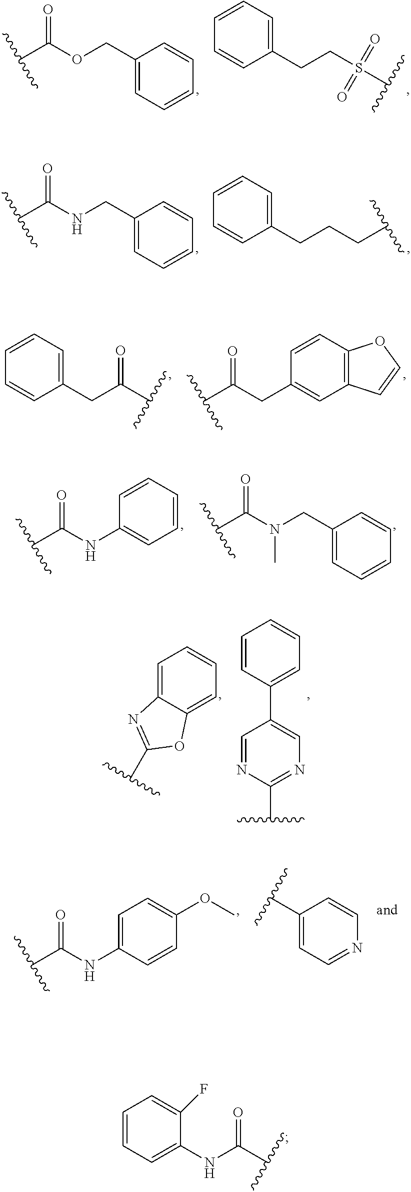

X is CR1R2; R1 is H; R2 is H; Y is O; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

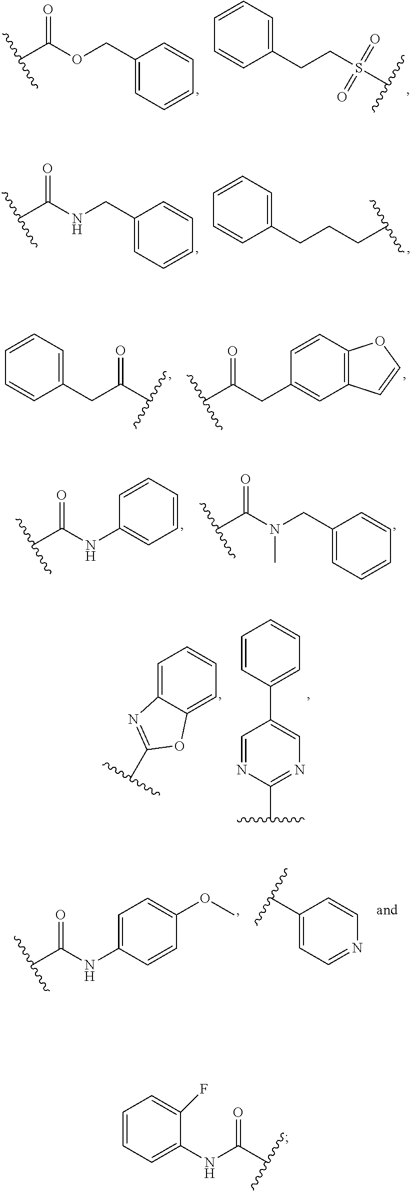

X is CR1R2; R1 is H; R2 is H; Y is O; and B is azaindole (particularly 7-azaindole), optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

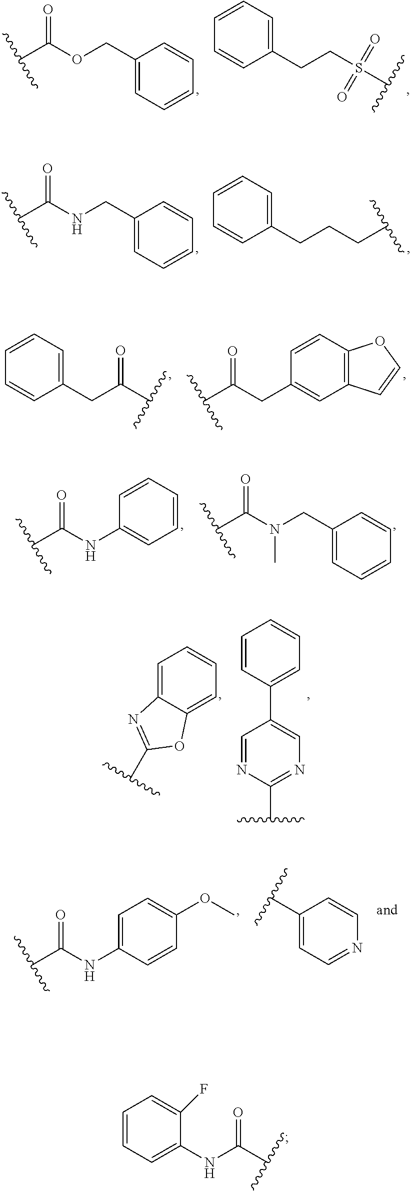

X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

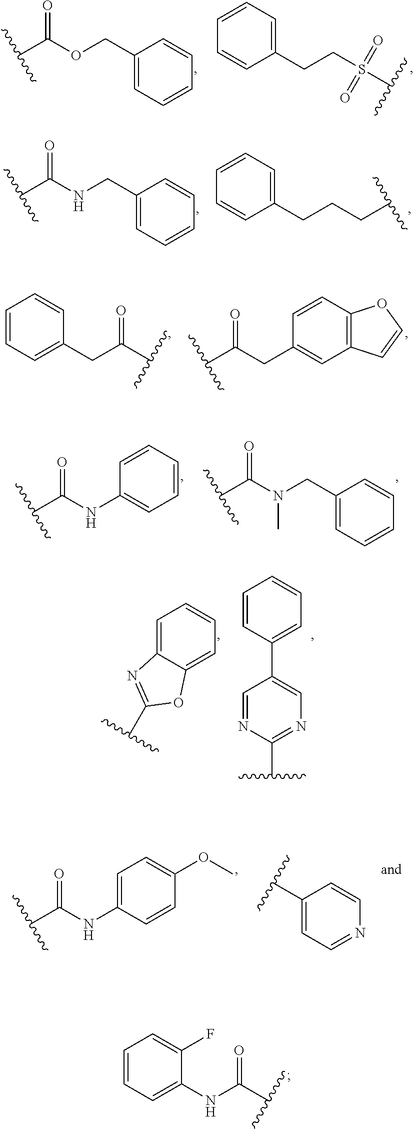

X is CR1R2; R1 is H; R2 is H; Y is O; and B is azaindole (particularly 7-azaindole), substituted with alkyl, and optionally substituted with 1 or 2 further substituents as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

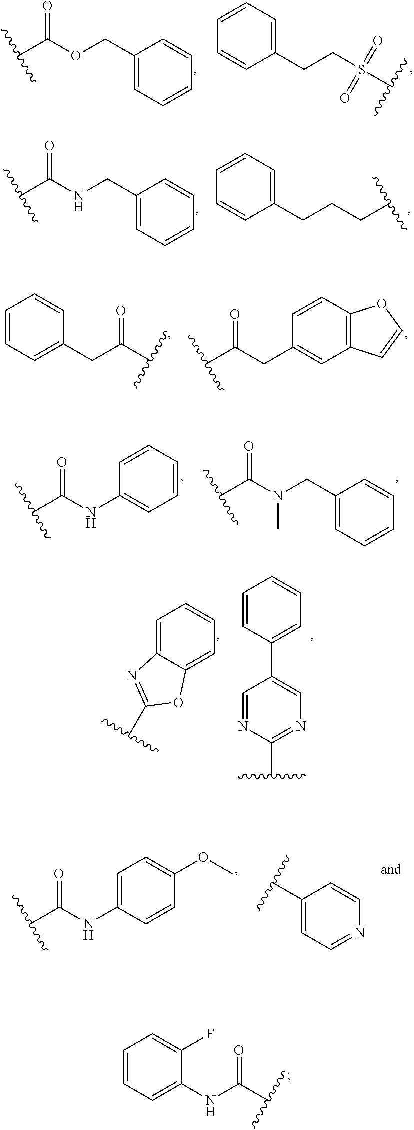

X is CR1R2; R1 is H; R2 is H; Y is O; and B is azaindole (particularly 7-azaindole), substituted with NH₂, and optionally substituted with 1 or 2 further substituents as for heteroaryl^{b}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

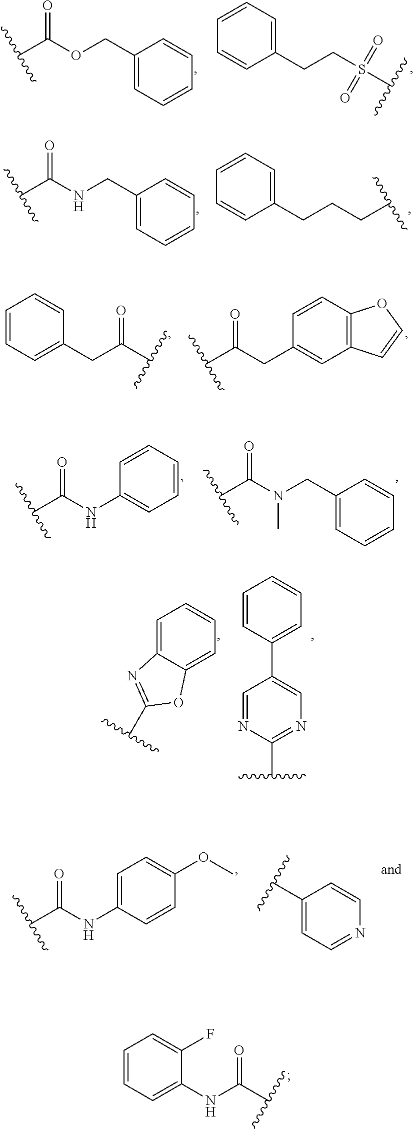

X is CR1R2; R1 is H; R2 is H; Y is O; and B is azaindole (particularly 7-azaindole), substituted with methyl, and optionally substituted with 1 or 2 further substituents as for heteroaryl^{a}.

Preferably, -V-Z- is selected from —CH₂—, —O—CH₂—, —CH₂—O— and —CH₂—CH₂—O—; X is CR1R2; R1 is H; R2 is H; Y is O; and B is heteroaryl^{a}.

More preferably, -V-Z- is selected from —CH₂—, —O—CH₂—, —CH₂—O— and —CH₂—CH₂—O—; X is CR1R2; R1 is H; R2 is H; Y is O; and B is azaindole (particularly 7-azaindole), optionally substituted as for heteroaryl^{a}.

Yet more preferably, -V-Z- is selected from —CH₂—, —O—CH₂—, —CH₂—O— and —CH₂—CH₂—O—; X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O— and —CH₂—CH₂—O—; X is CR1R2; R1 is H; R2 is H; Y is O; and B is azaindole (particularly 7-azaindole), substituted with alkyl, and optionally substituted with 1 or 2 further substituents as for heteroaryl^{a}.

Alternatively, -V-Z- is selected from —CH₂—, —O—CH₂—, —CH₂—O— and —CH₂—CH₂—O—; X is CR1R2; R1 is H; R2 is H; Y is O; and B is azaindole (particularly 7-azaindole), substituted with NH₂, and optionally substituted with 1 or 2 further substituents as for heteroaryl^{b}.

Preferably, -V-Z- is selected from —CH₂—, —O—CH₂—, —CH₂—O— and —CH₂—CH₂—O—; X is CR1R2; R1 is H; R2 is H; Y is O; and B is azaindole (particularly 7-azaindole), substituted with methyl, and optionally substituted with 1 or 2 further substituents as for heteroaryl^{a}.

More specifically, -V-Z- is selected from —CH₂—, —O—CH₂—, —CH₂—O— and —CH₂—CH₂—O—; X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, X is CR1R2; R1 is H; R2 is H; Y is O; and B is heteroaryl^{a}. More preferably, X is CR1R2; R1 is H; R2 is H; Y is O; and B is azaindole (particularly 7-azaindole), optionally substituted as for heteroaryl^{a}. Yet more preferably, X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}. Alternatively, X is CR1R2; R1 is H; R2 is H; Y is O; and B is azaindole (particularly 7-azaindole), substituted with alkyl, and optionally substituted with 1 or 2 further substituents as for heteroaryl^{a}. Alternatively, X is CR1R2; R1 is H; R2 is H; Y is O; and B is azaindole (particularly 7-azaindole), substituted with NH₂, and optionally substituted with 1 or 2 further substituents as for heteroaryl^{b}. Preferably, X is CR1R2; R1 is H; R2 is H; Y is O; and B is azaindole (particularly 7-azaindole), substituted with methyl, and optionally substituted with 1 or 2 further substituents as for heteroaryl^{a}. More specifically, X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂, and optionally substituted with 1 or 2 further substituents as for heteroaryl^{b}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂, and optionally substituted with 1 or 2 further substituents as for heteroaryl^{b}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), optionally substituted as for heteroaryl^{a}.

Yet more preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), substituted with NH₂, and optionally substituted with 1 or 2 further substituents as for heteroaryl^{b}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—; X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is azaindole (particularly 7-azaindole), optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
X is CR1R2; R1 is H; R2 is H; Y is O; and B is azaindole (particularly 7-azaindole), substituted with NH₂, and optionally substituted with 1 or 2 further substituents as for heteroaryl^{b}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀-aryl and (CH₂)₀₋₆-heteroaryl^{a};
X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

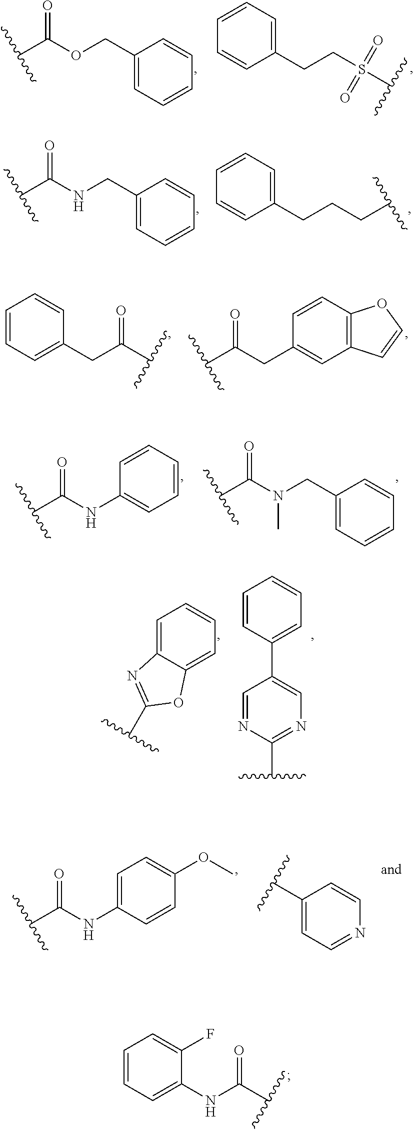

X is CR1R2; R1 is H; R2 is H; Y is O; and B is heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

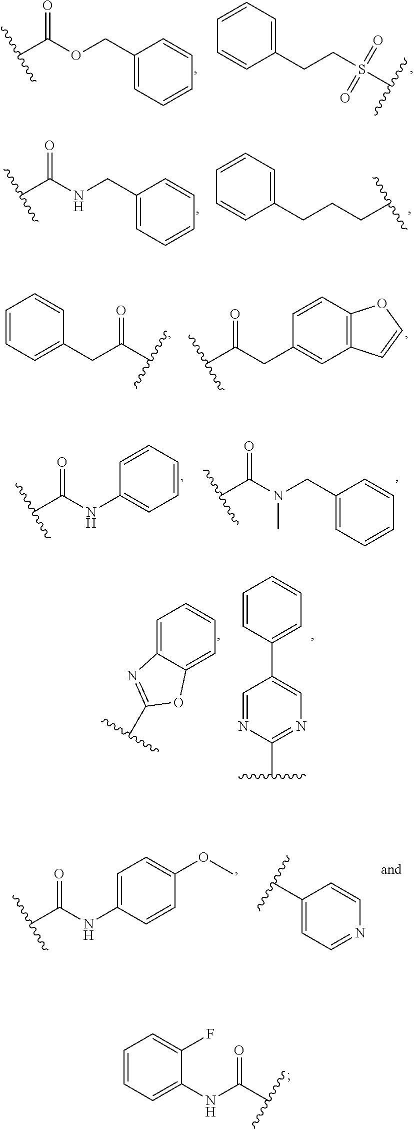

X is CR1R2; R1 is H; R2 is H; Y is O; and B is azaindole (particularly 7-azaindole), optionally substituted as for heteroaryl^{a}.

Yet more preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

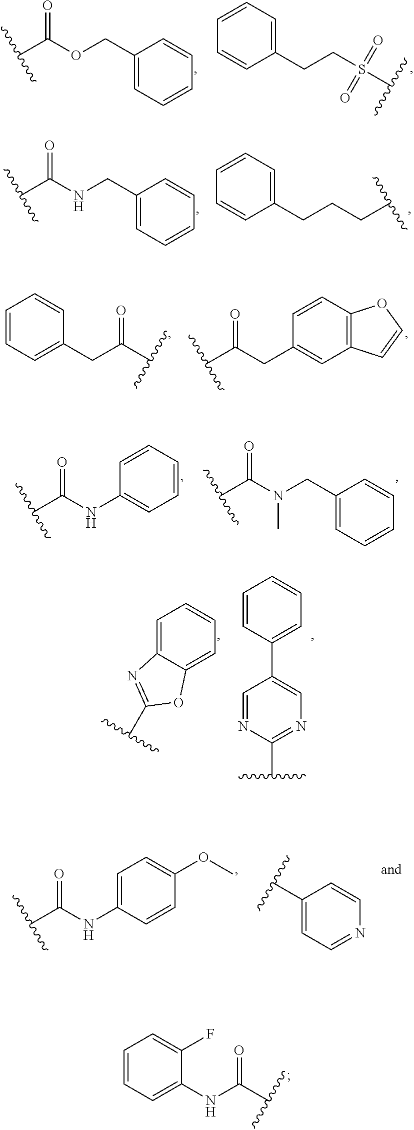

X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

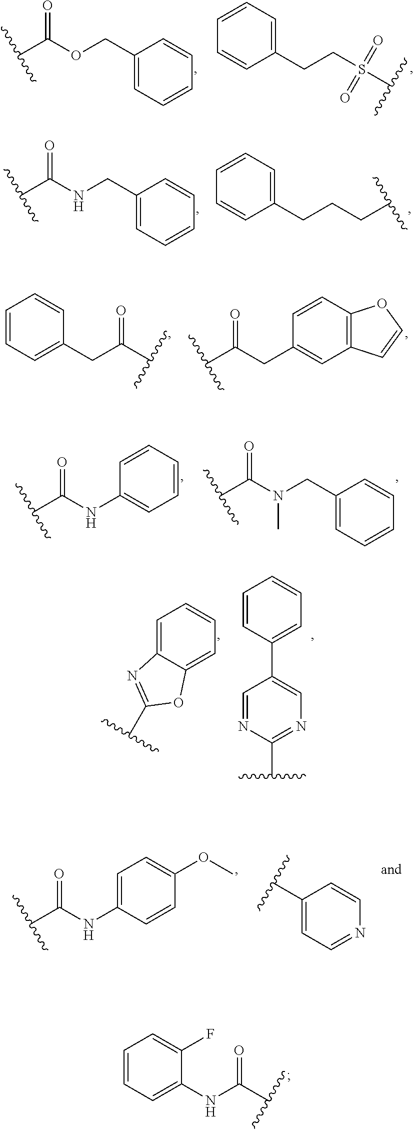

X is CR1R2; R1 is H; R2 is H; Y is O; and B is azaindole (particularly 7-azaindole), substituted with NH₂, and optionally substituted with 1 or 2 further substituents as for heteroaryl^{b}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:

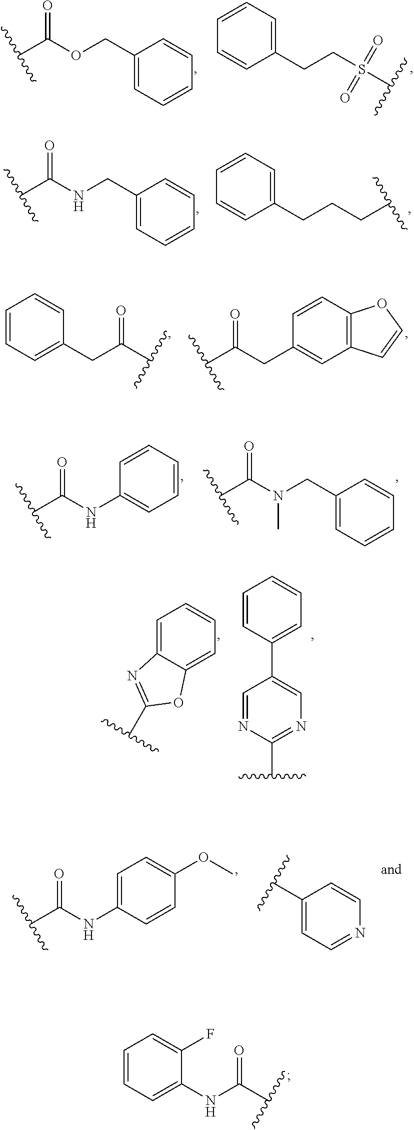

X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, -V-Z- is selected from —CH₂—, —O—CH₂—, —CH₂—O— and —CH₂—CH₂—O—; X is CR1R2; R1 is H; R2 is H; Y is O; and B is heteroaryl^{a}.

More preferably, -V-Z- is selected from —CH₂—, —O—CH₂—, —CH₂—O— and —CH₂—CH₂—O—; X is CR1R2; R1 is H; R2 is H; Y is O; and B is azaindole (particularly 7-azaindole), optionally substituted as for heteroaryl^{a}.

Yet more preferably, -V-Z- is selected from —CH₂—, —O—CH₂—, —CH₂—O— and —CH₂—CH₂—O—; X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}.

Alternatively, -V-Z- is selected from —CH₂—, —O—CH₂—, —CH₂—O— and —CH₂—CH₂—O—; X is CR1R2; R1 is H; R2 is H; Y is O; and B is azaindole (particularly 7-azaindole), substituted with NH₂, and optionally substituted with 1 or 2 further substituents as for heteroaryl^{b}.

More specifically, -V-Z- is selected from —CH₂—, —O—CH₂—, —CH₂—O— and —CH₂—CH₂—O—; X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

Preferably, X is CR1R2; R1 is H; R2 is H; Y is O; and B is heteroaryl^{a}. More preferably, X is CR1R2; R1 is H; R2 is H; Y is O; and B is azaindole (particularly 7-azaindole), optionally substituted as for heteroaryl^{a}. Yet more preferably, X is CR1R2; R1 is H; R2 is H; Y is O; and B is azaindole (particularly 7-azaindole), substituted with halo (e.g. chloro), and optionally substituted with 1 or 2 further substituents as for heteroaryl^{a}. Alternatively, X is CR1R2; R1 is H; R2 is H; Y is O; and B is azaindole (particularly 7-azaindole), substituted with NH₂, and optionally substituted with 1 or 2 further substituents as for heteroaryl^{b}. More specifically, X is CR1R2; R1 is H; R2 is H; Y is O; and B is azaindole (particularly 7-azaindole), substituted with NH₂ and halo (e.g. chloro), and optionally 1 further substituent as for heteroaryl^{a}.

AW- can be selected from:
—(CH₂)₀₋₆—(CHR15)-(CH₂)₀₋₆-A, —(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—NR12-(CH₂)₁₋₆—C(═O)-A, —(CH₂)₀₋₆—NH—C(═O)—(CH₂)₀₋₆-A, —C(═O)NR12-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A and —SO₂—NH-A.

R15 is selected from alkyl, halo, CF₃, CN, OH, alkoxy, NR13R14, and CONR13R14. R15 can be alkyl (e.g. methyl or ethyl). R15 can be halo (e.g. fluoro or chloro). R15 can be CF₃. R15 can be CN. R15 can be OH. R15 can be alkoxy (e.g. methoxy or ethoxy). R15 can be NR13R14, particularly NH₂. R15 can be CONR13R14, particularly CONH2.

AW- can be selected from:
—(CHR12)-A, —O—(CHR12)-A, —(CH₂)₀₋₅-A, —(CH₂)₀₋₅—O—(CH₂)₀₋₅-A, —(CH₂)₀₋₅—NH—(CH₂)₀₋₅-A, —(CH₂)₀₋₅—NR12-(CH₂)₁₋₅—C(═O)-A, —(CH₂)₀₋₅—NH—C(═O)—(CH₂)₀₋₅-A, —C(═O)NR12-(CH₂)₀₋₅-A, —(CH₂)₀₋₅—C(═O)—(CH₂)₀₋₅-A, —(CH₂)₀₋₅-(phenyl)-(CH₂)₀₋₅-A, —NH—SO₂-A and —SO₂—NH-A.

AW- can be selected from:
—(CHR12)-A, —O—(CHR12)-A, —(CH₂)₀₋₄-A, —(CH₂)₀₋₄—O—(CH₂)₀₋₄-A, —(CH₂)₀₋₄—NH—(CH₂)₀₋₄-A, —(CH₂)₀₋₄—NR12-(CH₂)₁₋₄—C(═O)-A, —(CH₂)₀₋₄—NH—C(═O)—(CH₂)₀₋₄-A, —C(═O)NR12-(CH₂)₀₋₄-A, —(CH₂)₀₋₄—C(═O)—(CH₂)₀₋₄-A, —(CH₂)₀₋₄-(phenyl)-(CH₂)₀₋₄-A, —NH—SO₂-A and —SO₂—NH-A.

AW- can be selected from:
—(CHR12)-A, —O—(CHR12)-A, —(CH₂)₀₋₃-A, —(CH₂)₀₋₃—O—(CH₂)₀₋₃-A, —(CH₂)₀₋₃—NH—(CH₂)₀₋₃-A, —(CH₂)₀₋₃—NR12-(CH₂)₁₋₃—C(═O)-A, —(CH₂)₀₋₃—NH—C(═O)—(CH₂)₀₋₃-A, —C(═O)NR12-(CH₂)₀₋₃-A, —(CH₂)₀₋₃—C(═O)—(CH₂)₀₋₃-A, —(CH₂)₀₋₃-(phenyl)-(CH₂)₀₋₃-A, —NH—SO₂-A and —SO₂—NH-A.

Preferably AW- can be selected from —(CHR12)-A, —(CH₂)₀₋₆—C(═O)—(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₃-(phenyl)-(CH₂)₀₋₃-A and —(CH₂)₀₋₆—NH—C(═O)—(CH₂)₀₋₆-A. AW- can be selected from —(CHR12)-A, —(CH₂)₀₋₆—C(═O)—(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₃-(phenyl)-(CH₂)₀₋₃-A and —(CH₂)₀₋₆—NH—C(═O)—(CH₂)₀₋₆-A.

Preferably, AW- can be selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A.

More specifically, AW- can be selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A.

Preferably AW- is selected from —(CHR12)₀₋₆-A and —(CH₂)₀₋₆—O—(CH₂)₀₋₆-A. AW- can be selected from —(CHR12)₀₋₃-A and —(CH₂)₀₋₃—O—(CH₂)₀₋₃-A.

Preferably AW- is selected from —(CHR12)-A, —(CH₂)₀₋₆—C(═O)—(CH₂)₀₋₆-A, and —(CH₂)₀₋₆—O—(CH₂)₀₋₆-A. AW- can be selected from —(CHR12)₀₋₃-A, —(CH₂)₀₋₃—C(═O)—(CH₂)₀₋₃-A, and —(CH₂)₀₋₃—O—(CH₂)₀₋₃-A.

Preferably AW- is selected from —(CHR12)-A and —(CH₂)₀₋₆—O—(CH₂)₀₋₆-A. AW- can be selected from —(CHR12)₀₋₃-A and —(CH₂)₀₋₃—O—(CH₂)₀₋₃-A.

Preferably AW- can be —(CH₂)₀₋₆—O—(CH₂)₀₋₆-A.

Preferably AW- can be —CH₂—O-A.

AW- can be —(CHR12)-A. AW- can be —(CH₂)₀₋₆—C(═O)—(CH₂)₀₋₆-A. AW- can be —(CH₂)₀₋₆—O—(CH₂)₀₋₆-A. AW- can be —(CH₂)₀₋₆—NH—(CH₂)₀₋₆-A. AW- can be —(CH₂)₀₋₆—NH—C(═O)—(CH₂)₀₋₆-A. AW- can be —(CH₂)₀₋₃-(phenyl)-(CH₂)₀₋₃-A.

AW- can be —(CH₂)₀₋₆—O—(CH₂)₀₋₆-A.

AW- can be —CH₂—O-A.

AW- can be —(CHR12)₀₋₆-A. AW- can be —(CHR12)₀₋₃-A. AW- can be —(CHR12)-A. AW- can be —(CH₂)₀₋₆-A. AW- can be —(CH₂)₀₋₃-A. AW- can be —(CH₂)-A.

AW- can be —(CHR12)-A. AW- can be —C(═O)—(CH₂)₀₋₆-A. AW- can be —(CH₂)₀₋₆—C(═O)-A. AW- can be —C(═O)—(CH₂)₀₋₃-A. AW- can be —(CH₂)₀₋₃—C(═O)-A. AW- can be —C(═O)-A. AW- can be —(CH₂)₀₋₆-A. AW- can be —(CH₂)₀₋₃-A. AW- can be —(CH₂)-A.

AW- can be —(CHR12)-A. AW- can be —(CH₂)₀₋₆-A. AW- can be —(CH₂)₀₋₃-A. AW- can be —(CH₂)-A.

AW- can be —(CH₂)₀₋₆—O—(CH₂)₀₋₆-A. AW- can be —(CH₂)₀₋₃—O—(CH₂)₀₋₆-A. AW- can be —(CH₂)₀₋₆—O—(CH₂)₀₋₃-A. AW- can be —(CH₂)₀₋₃—O—(CH₂)₀₋₃-A. AW can be —(CH₂)—O—(CH₂)₀₋₆-A. AW- can be —(CH₂)₀₋₆—O-A. AW can be —(CH₂)₀₋₆—O—(CH₂)-A. AW can be —O—(CH₂)₀₋₆-A. AW- can be —(CH₂)₀₋₃—O-A. AW- can be —O—(CH₂)₀₋₃-A. AW- can be —(CH₂)—O—(CH₂)-A. AW- can be —O—(CH₂)-A. Preferably AW- is —(CH₂)—O-A.

AW- can be —(CH₂)₀₋₆—O—(CH₂)₀₋₆-A. AW- can be —(CH₂)₀₋₃—O—(CH₂)₀₋₆-A. AW- can be —(CH₂)₀₋₆—O—(CH₂)₀₋₃-A. AW- can be —(CH₂)₀₋₃—O—(CH₂)₀₋₃-A. AW- can be —(CH₂)₀₋₆—O-A. AW can be —O—(CH₂)₀₋₆-A. AW- can be —(CH₂)₀₋₃—O-A. AW- can be —O—(CH₂)₀₋₃-A. AW- can be —(CH₂)—O—(CH₂)-A. AW- can be —O—(CH₂)-A. Preferably AW- is —(CH₂)—O-A.

A can be a 4- to 15-membered mono-, bi-, or tri-cyclic ring system, containing one N ring member and optionally one, two or three further ring members independently selected from N, O and S, optionally wherein the ring system is substituted, where possible, with 1, 2, 3 or 4 substituents independently C(═O)OR13, C(═O)NR13R14, NR13R14, CF₃, CN; wherein when A is a bicyclic ring system, the bicyclic ring system is fused, bridged or spiro; wherein when A is a tricyclic ring system, each of the three rings in the tricyclic ring system is either fused, bridged or spiro to at least one of the other rings in the tricyclic ring system.

A can be a 4- to 15-membered mono-, bi-, or tri-cyclic ring system, containing one N ring member and optionally one, two or three further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; wherein when A is a tricyclic ring system, each of the three rings in the tricyclic ring system is either fused, bridged or spiro to at least one of the other rings in the tricyclic ring system.

A can be a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro.

A can be a 4- to 12-membered mono- or bi-cyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro. A can be a 4- to 12-membered mono- or bi-cyclic ring system, containing one N ring member and optionally one or two further ring members independently selected from N, optionally wherein the ring system is substituted, where possible, with 1, 2, 3 or 4 substituents independently selected from alkyl and cycloalkyl; wherein when A is a bicyclic ring system, the bicyclic ring system is fused, bridged or spiro. A can be a 4- to 12-membered mono- or bi-cyclic ring system, containing one N ring member and optionally one further ring member independently selected from N, optionally wherein the ring system is substituted, where possible, with 1, 2, 3 or 4 substituents independently selected from alkyl and cycloalkyl; wherein when A is a bicyclic ring system, the bicyclic ring system is fused, bridged or spiro.

A can be a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl.

A can be a 6-membered monocyclic ring system containing one N ring member, wherein the ring system is substituted with 1 substituent selected from alkyl and cycloalkyl. More preferably, A is a 6-membered monocyclic ring system containing one N ring member, wherein the ring system is substituted with 1 alkyl substituent selected from methyl, ethyl, iso-propyl and cyclopropyl. Preferably, the 6-membered monocyclic ring system containing one N ring member is joined to W at the carbon para to the nitrogen.

A can be a 6-membered monocyclic ring system containing one N ring member and optionally one further ring member selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. Preferably, A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. More preferably, A is a 6-membered monocyclic ring system containing one N ring member, optionally wherein the ring system is substituted, where possible, with 1 or 2 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. Most preferably, A is a 6-membered monocyclic ring system containing one N ring member, wherein the ring system is substituted with 2 substituents independently selected from alkyl and oxo. For example, A can be

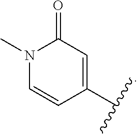 [SMILES: *c1ccn(C)c(=O)c1]

A can be a 4- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein the bicyclic ring system is fused, bridged or spiro.

A can be a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein the bicyclic ring system is fused, bridged or spiro.

A can be a fused 6- to 12-membered bicyclic ring system containing one N ring member and optionally one, two or three further ring members independently selected from N, O and S, wherein the fused ring system consists of an aromatic ring fused to a non-aromatic ring, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a fused 6- to 12-membered bicyclic ring system containing one N ring member and optionally one, two or three further ring members independently selected from N, O and S, wherein the fused ring system consists of an aromatic ring fused to a non-aromatic ring, optionally wherein the ring system is substituted, where possible, with 1, 2, 3 or 4 substituents independently selected from alkyl and CF₃.

A can be a fused 6- to 12-membered bicyclic ring system containing one N ring member and optionally one, two or three further ring members independently selected from N, O and S, wherein the fused ring system consists of a 5-membered aromatic ring fused to a 6-membered non-aromatic ring, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a fused 6- to 12-membered bicyclic ring system containing one N ring member and optionally one, two or three further ring members independently selected from N, O and S, wherein the fused ring system consists of a 5-membered aromatic ring fused to a 6-membered non-aromatic ring, optionally wherein the ring system is substituted, where possible, with 1, 2, 3 or 4 substituents independently selected from alkyl and CF₃.

A can be a 9- or 10-membered bicyclic ring system (particularly 9-membered), containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro.

A can be a fused 9- or 10-membered bicyclic ring system (particularly 9-membered) containing one N ring member and optionally one, two or three further ring members independently selected from N, O and S, wherein the fused ring system consists of an aromatic ring fused to a non-aromatic ring, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a fused 9- or 10-membered bicyclic ring system (particularly 9-membered) containing one N ring member and optionally one, two or three further ring members independently selected from N, O and S, wherein the fused ring system consists of an aromatic ring fused to a non-aromatic ring, optionally wherein the ring system is substituted, where possible, with 1, 2, 3 or 4 substituents independently selected from alkyl and CF₃.

A can be a fused 9- or 10-membered bicyclic ring system (particularly 9-membered) containing one N ring member and optionally one, two or three further ring members independently selected from N, O and S, wherein the fused ring system consists of a 5-membered aromatic ring fused to a 6-membered non-aromatic ring, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a fused 9- or 10-membered bicyclic ring system (particularly 9-membered) containing one N ring member and optionally one, two or three further ring members independently selected from N, O and S, wherein the fused ring system consists of a 5-membered aromatic ring fused to a 6-membered non-aromatic ring, optionally wherein the ring system is substituted, where possible, with 1, 2, 3 or 4 substituents independently selected from alkyl and CF₃.

A can be a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently C(═O)OR13, C(═O)NR13R14, NR13R14, CF₃, CN. One of the rings in the fused bicyclic ring system can be aromatic. Both of the rings in the fused bicyclic ring system can be aromatic.

A can be a fused 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. One of the rings in the fused bicyclic ring system can be aromatic. Both of the rings in the fused bicyclic ring system can be aromatic.

A can be selected from:

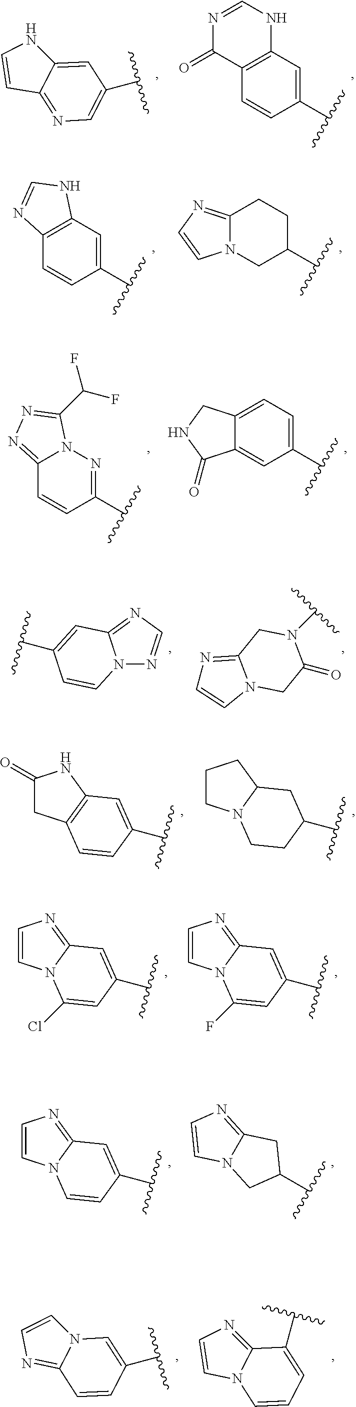

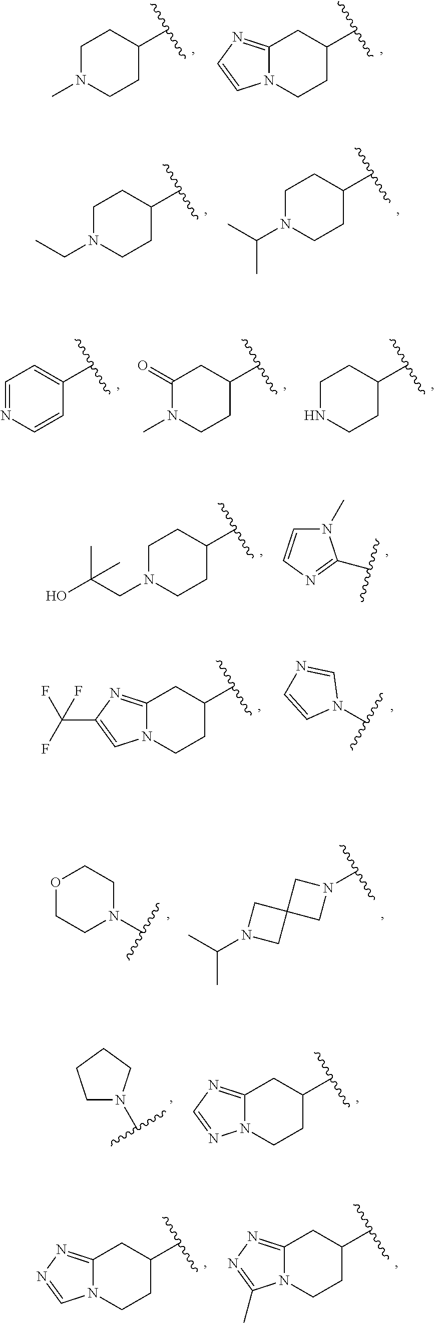

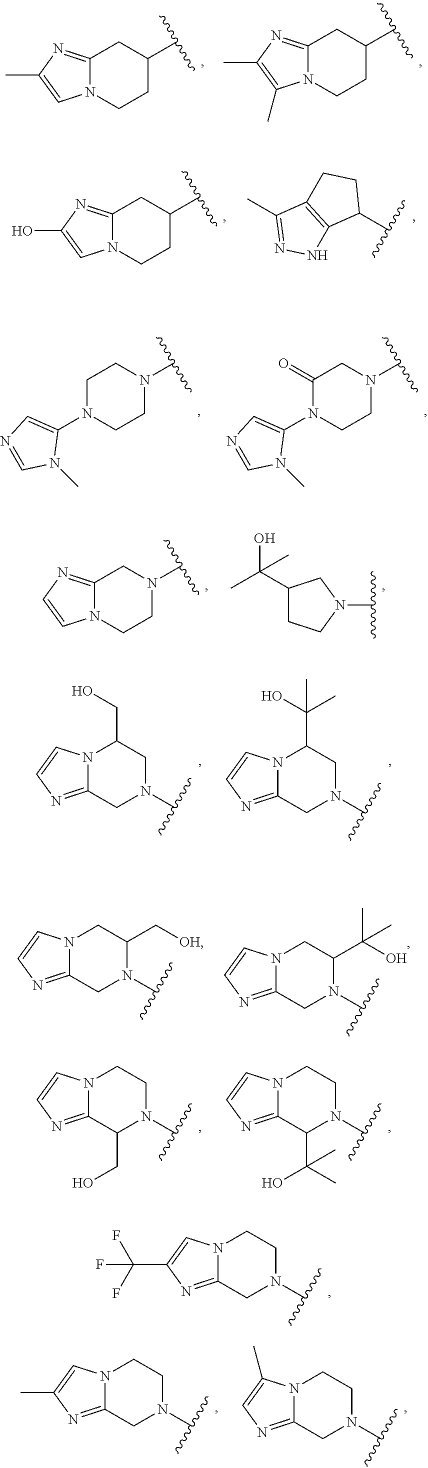

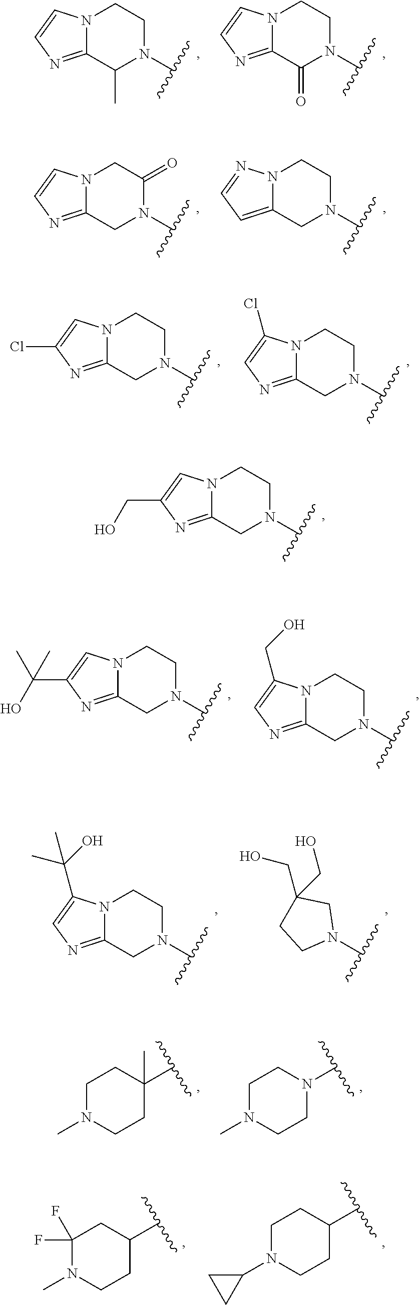

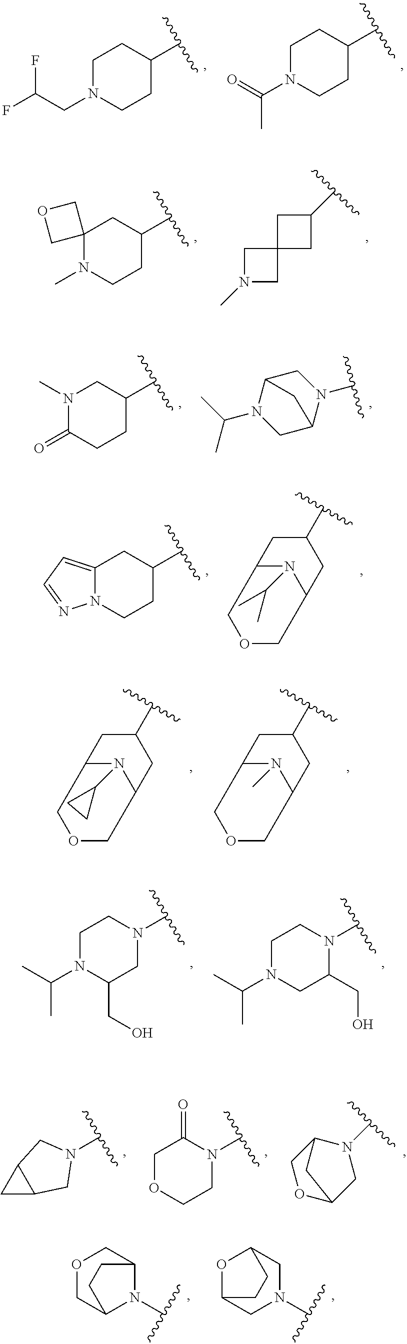

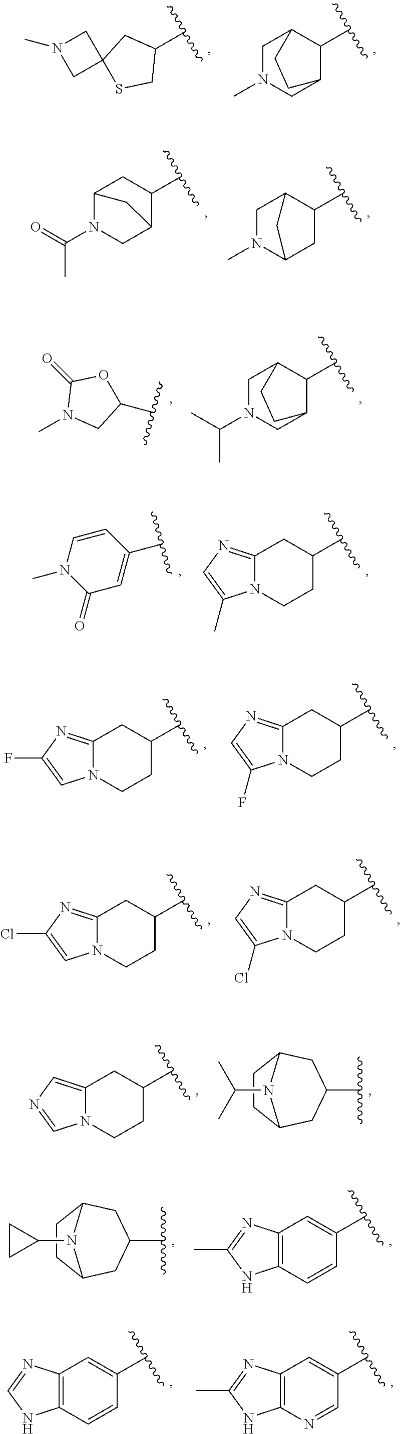

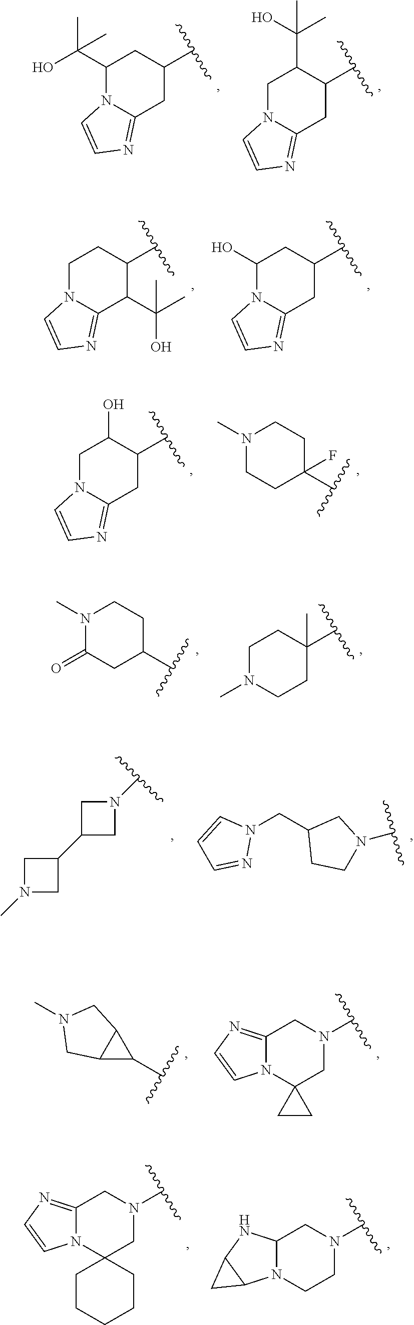

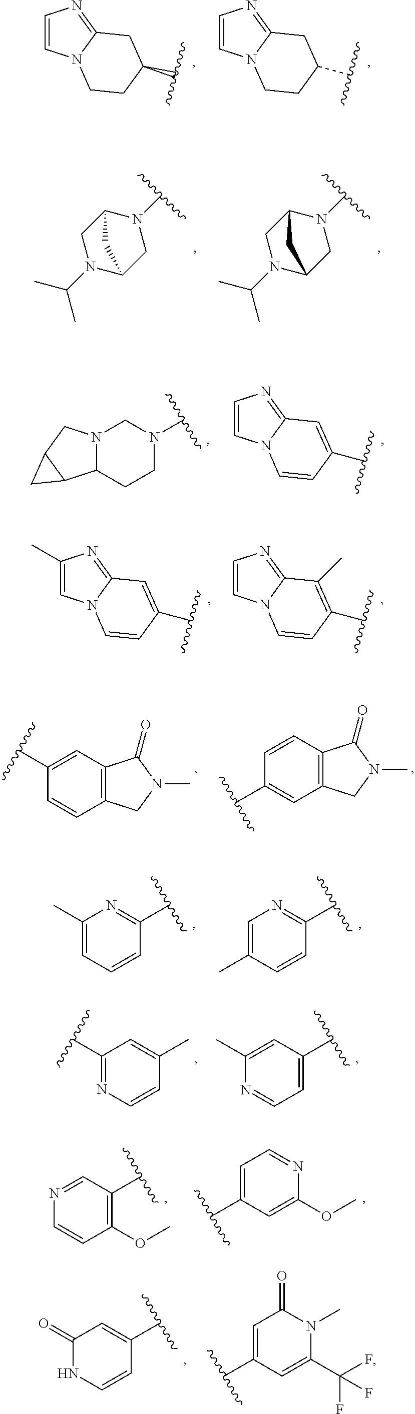

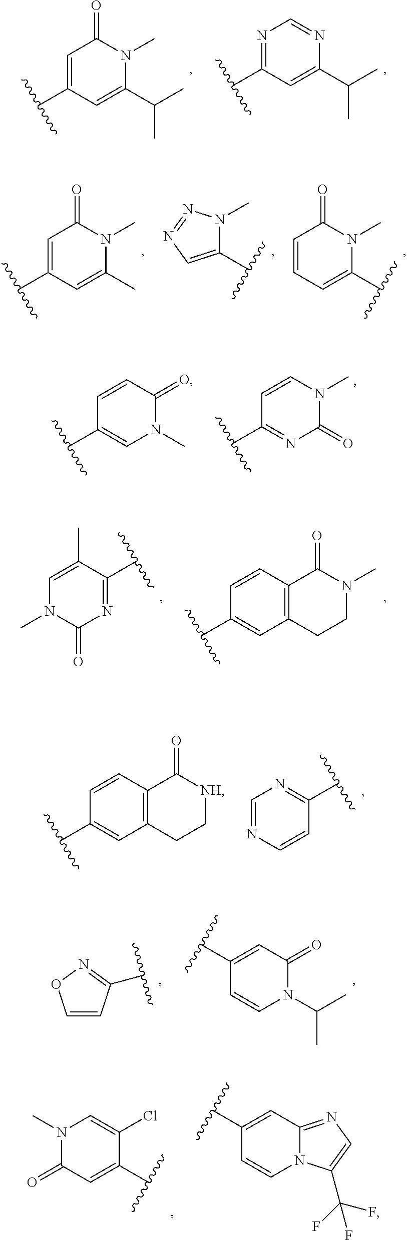

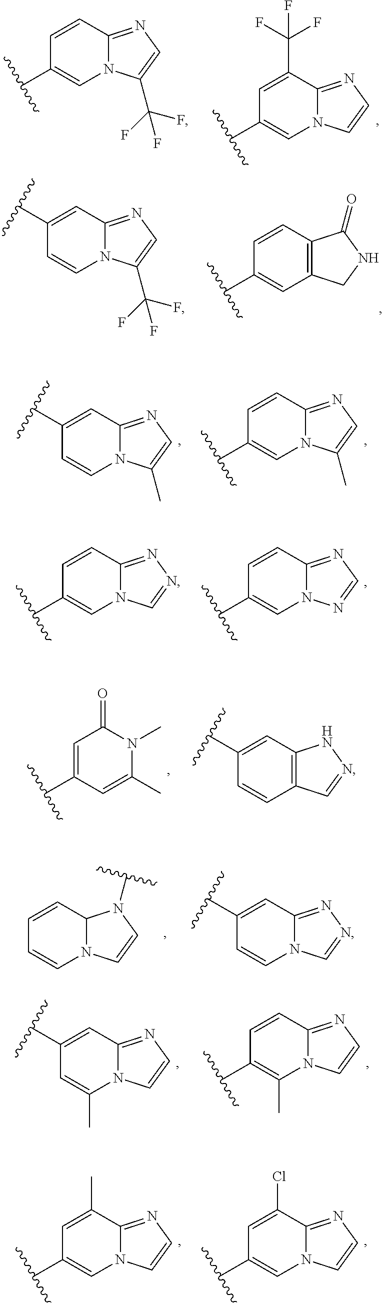

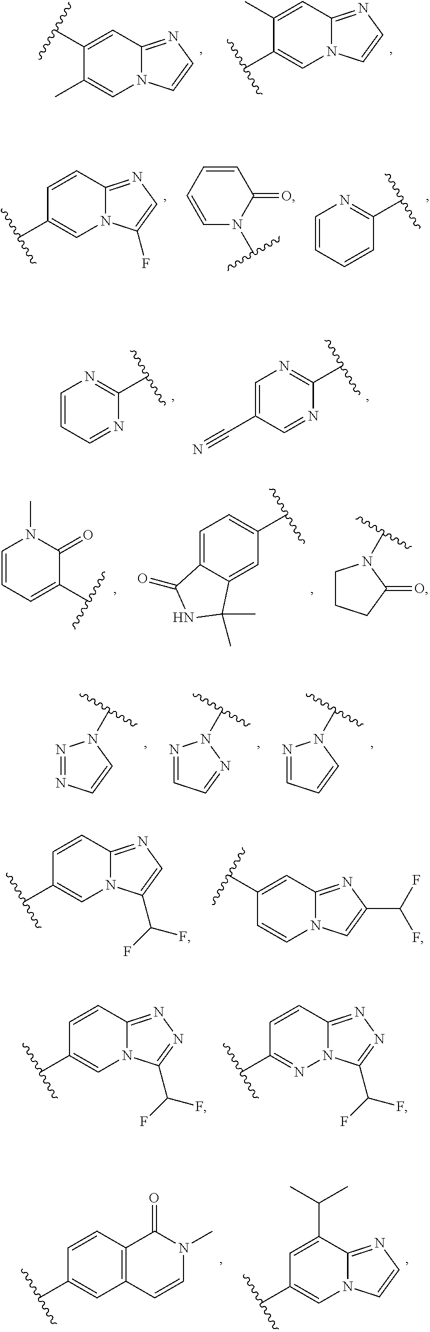

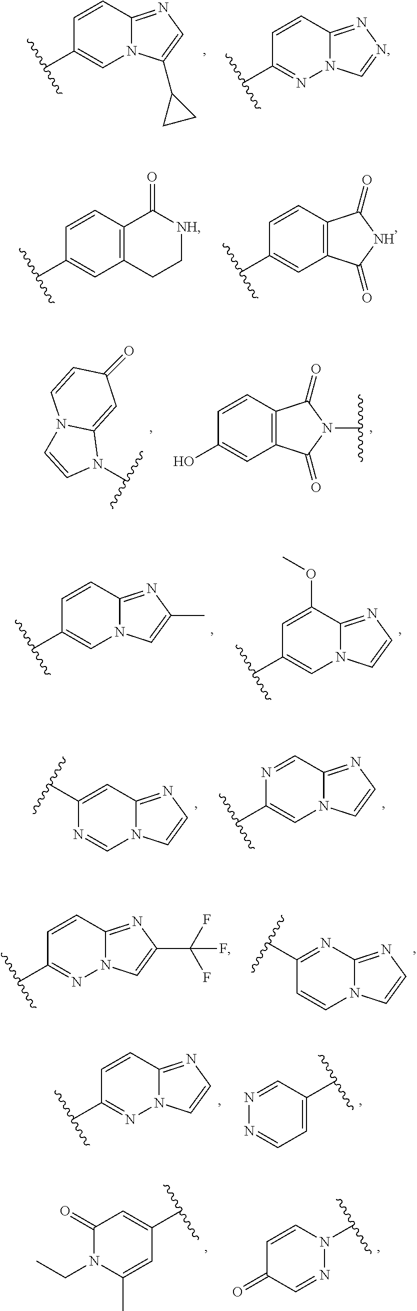

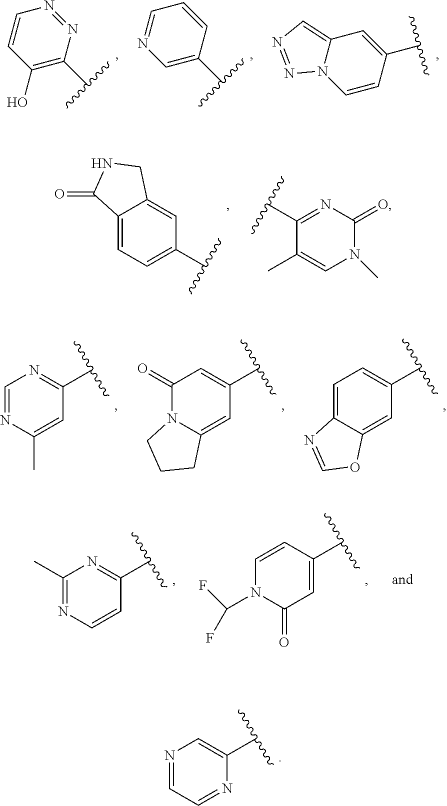

Preferably A can be selected from:

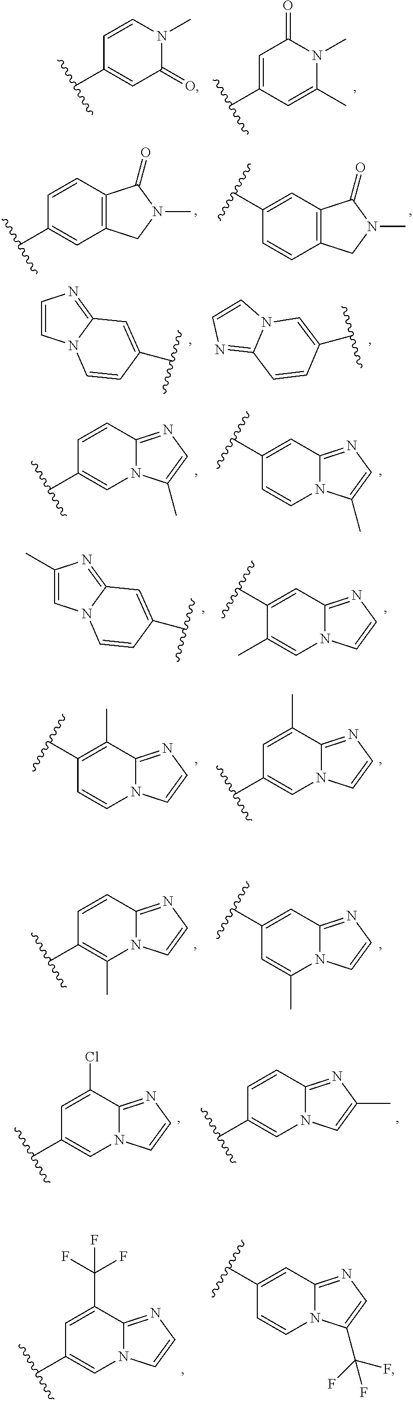

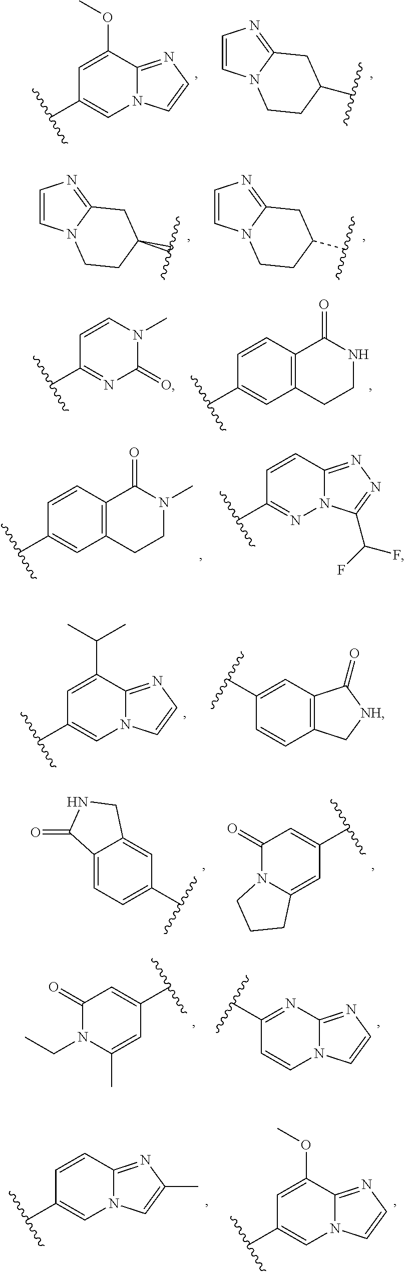

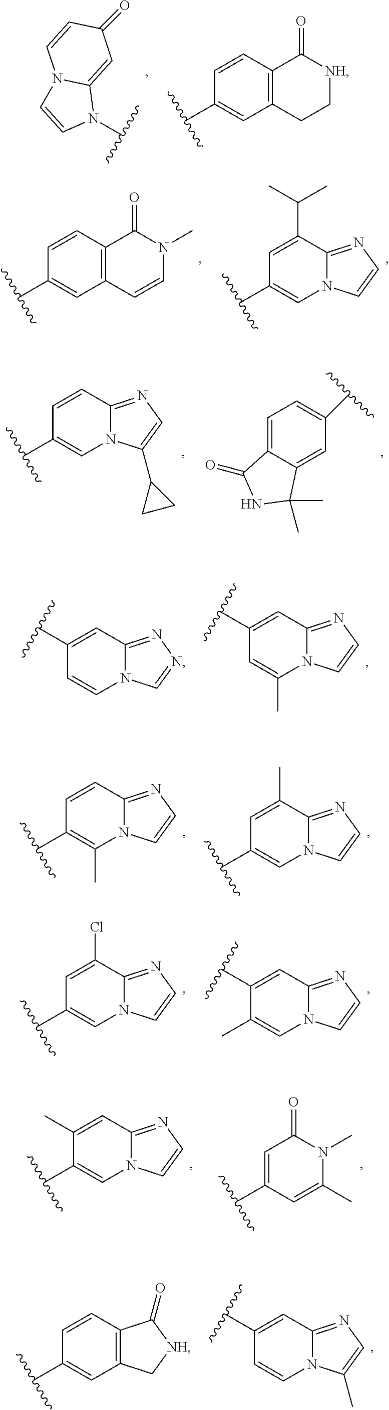

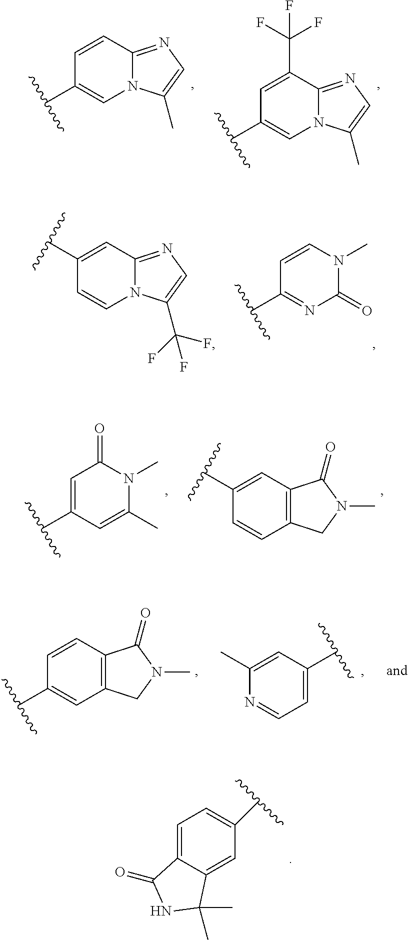

More preferably A can be selected from:

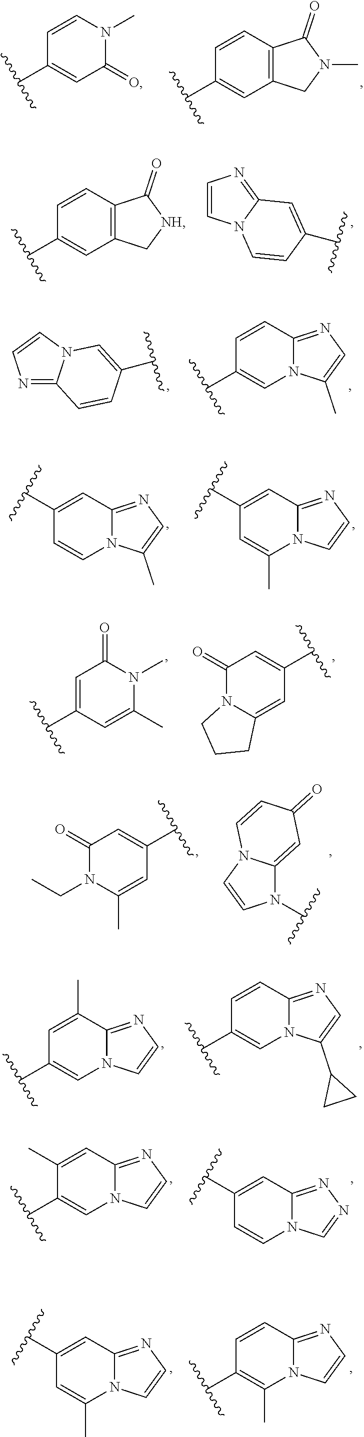

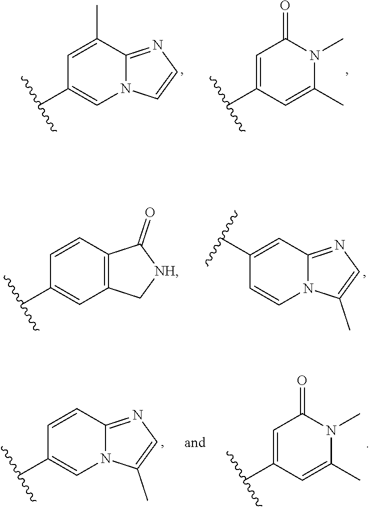

More preferably A can be selected from:

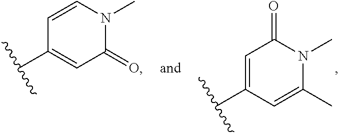

Preferably A can be

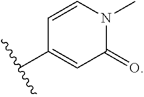 [SMILES: *c1ccn(C)c(=O)c1]

Alternatively A can be selected from:

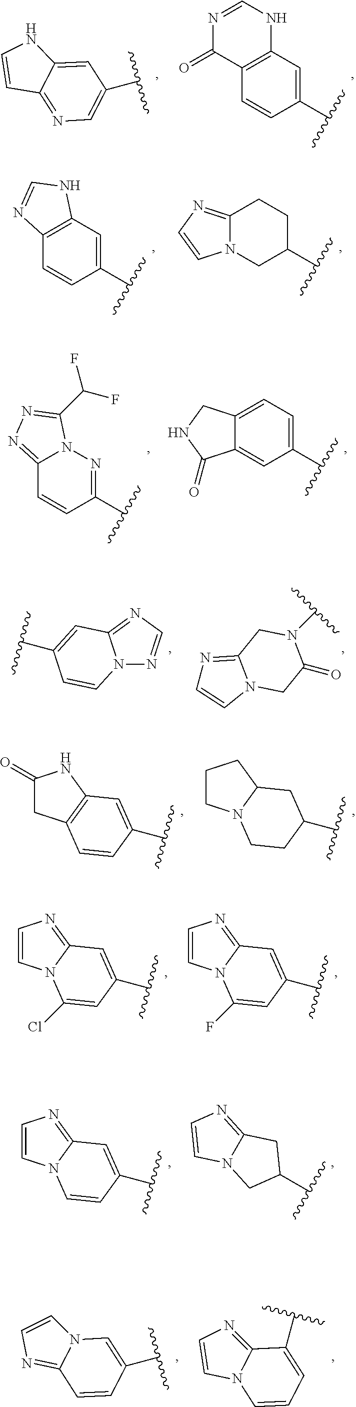

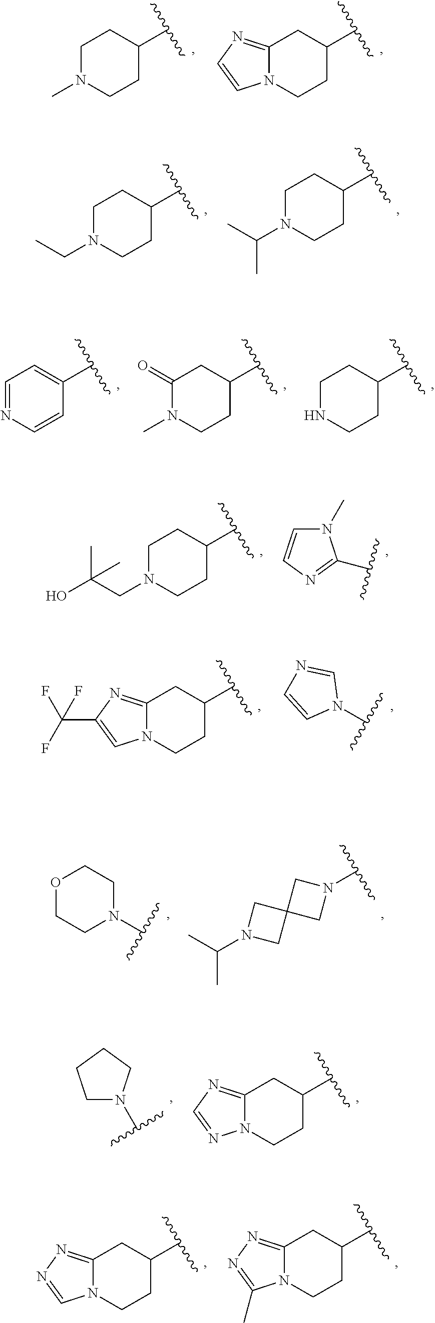

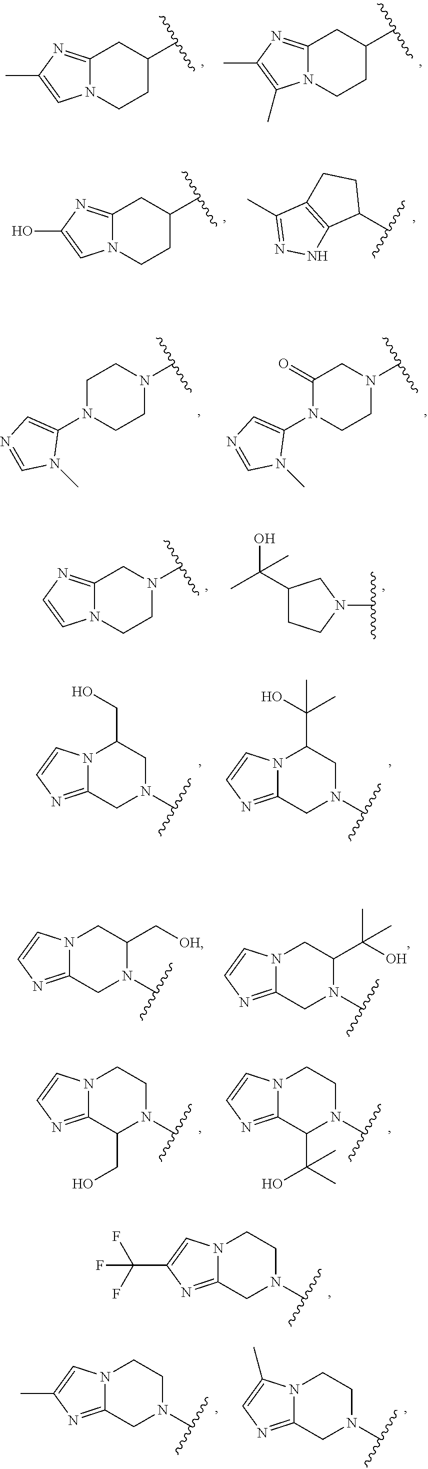

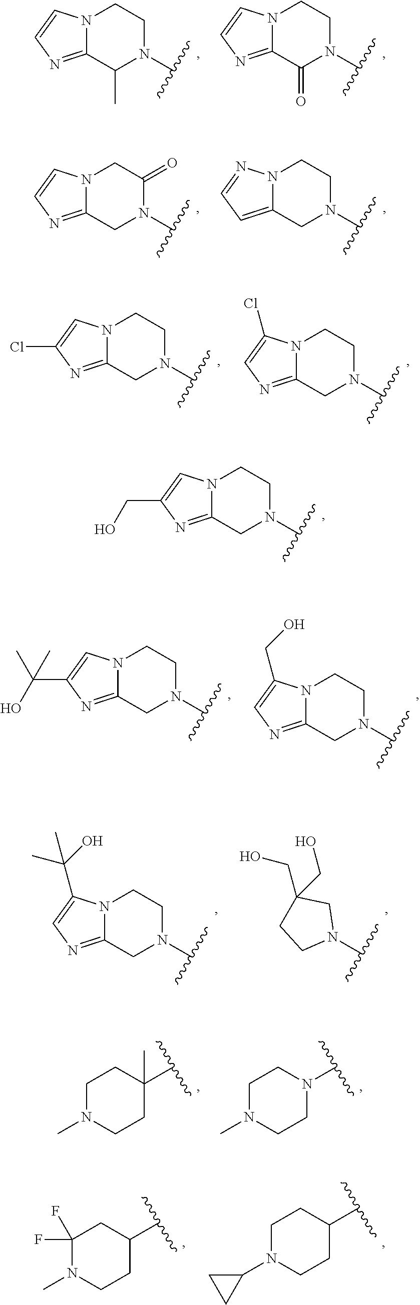

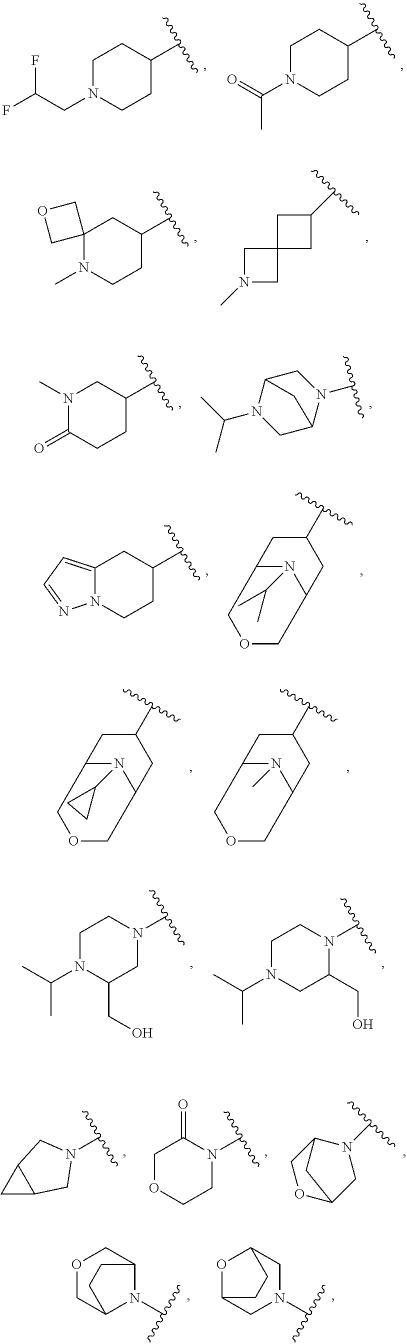

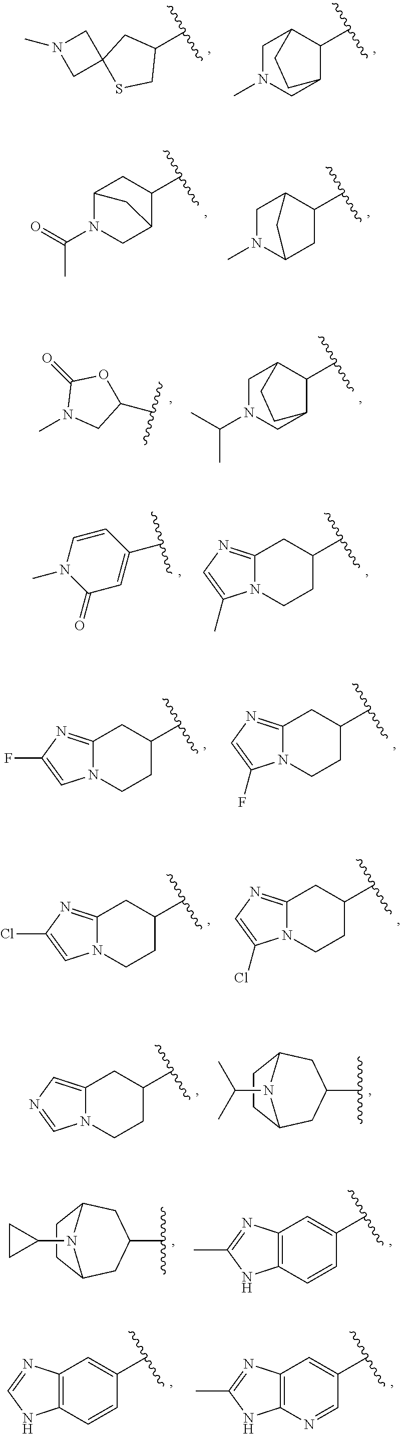

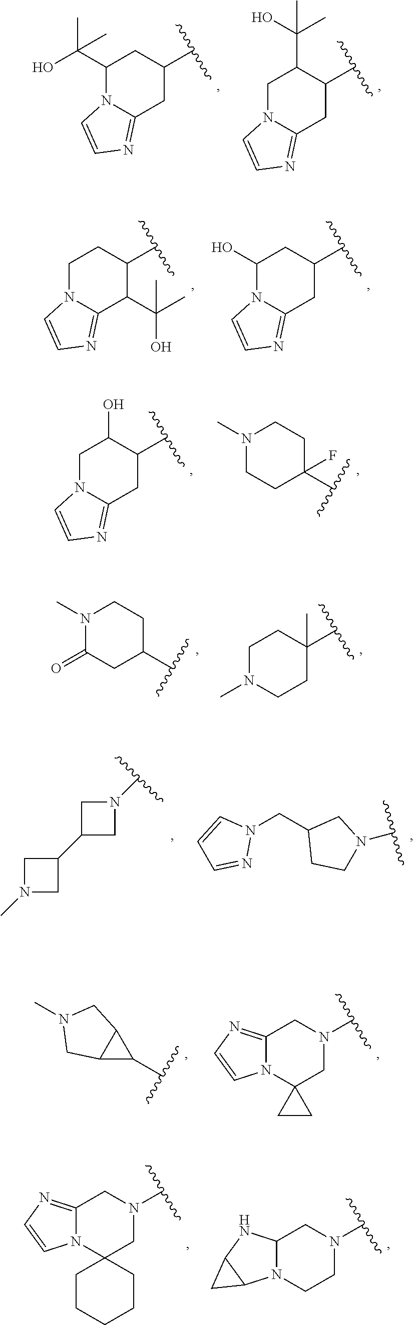

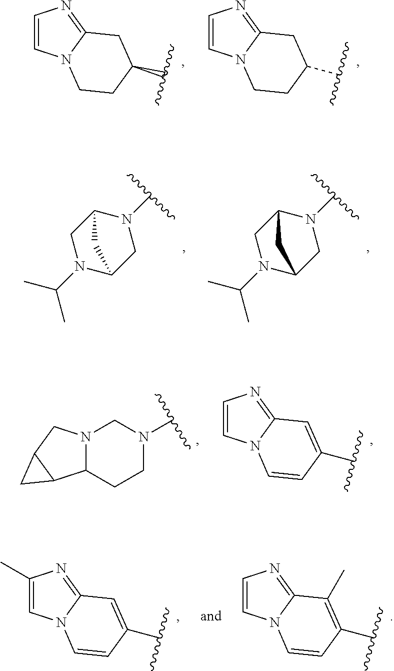

A can be selected from:

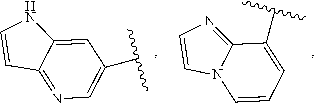 [SMILES: *c1cnc2cc[nH]c2c1 ; *c1cccn2ccnc12]

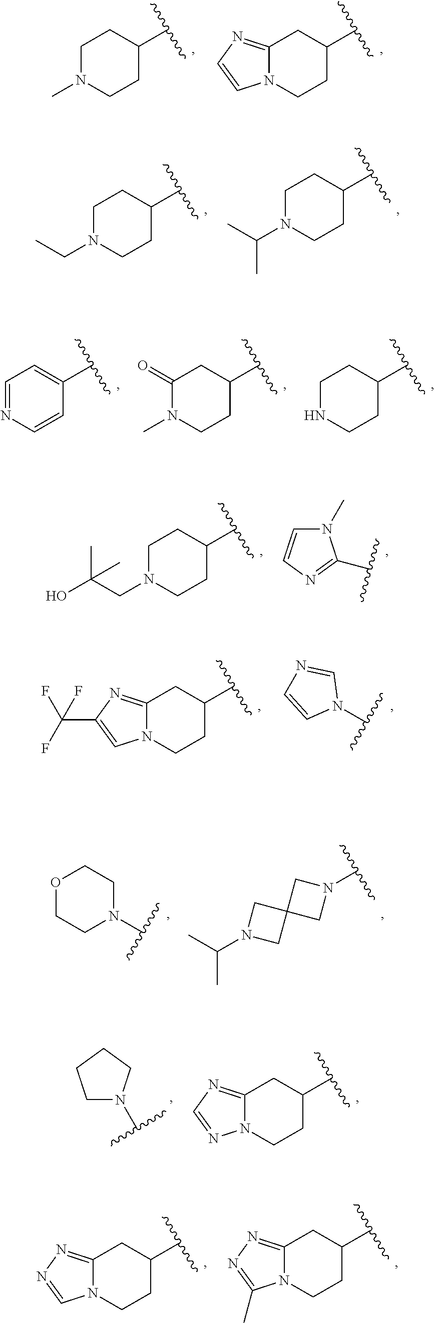

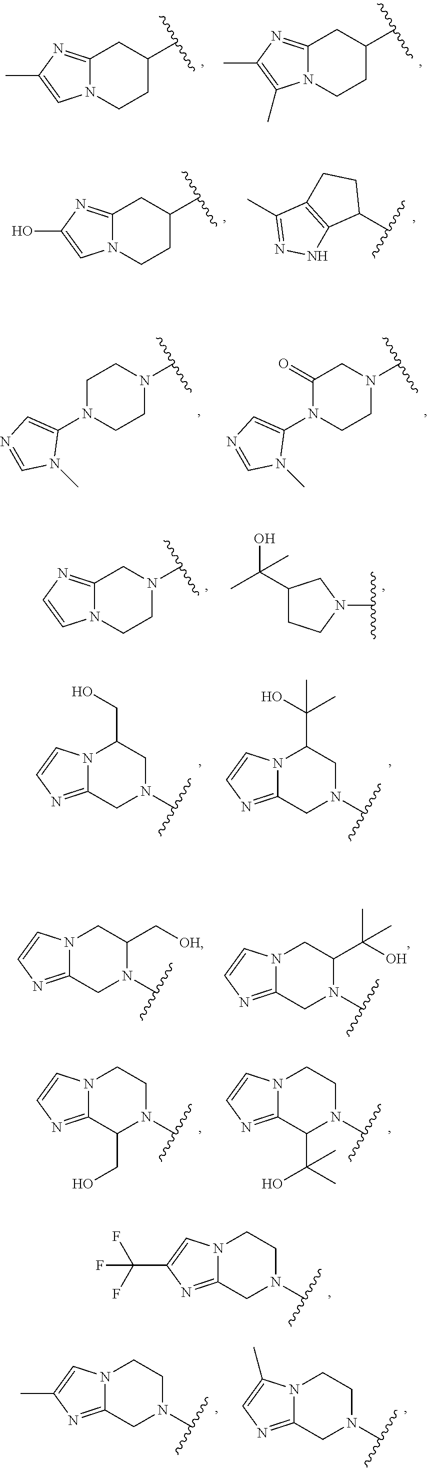

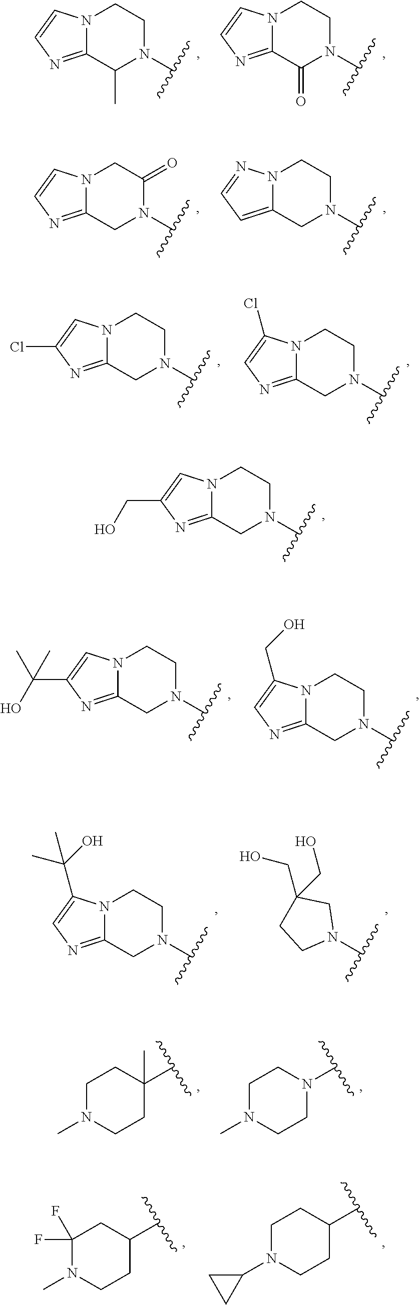

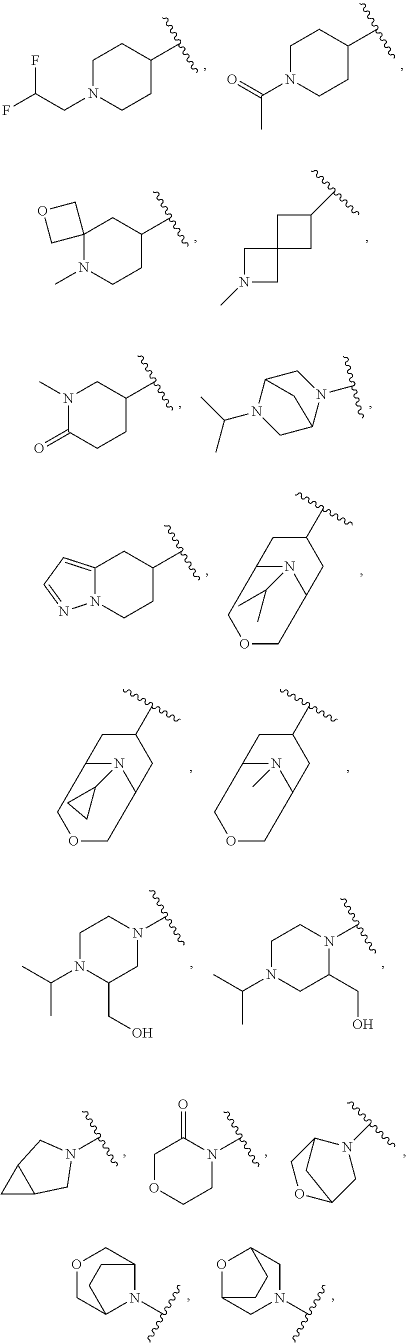

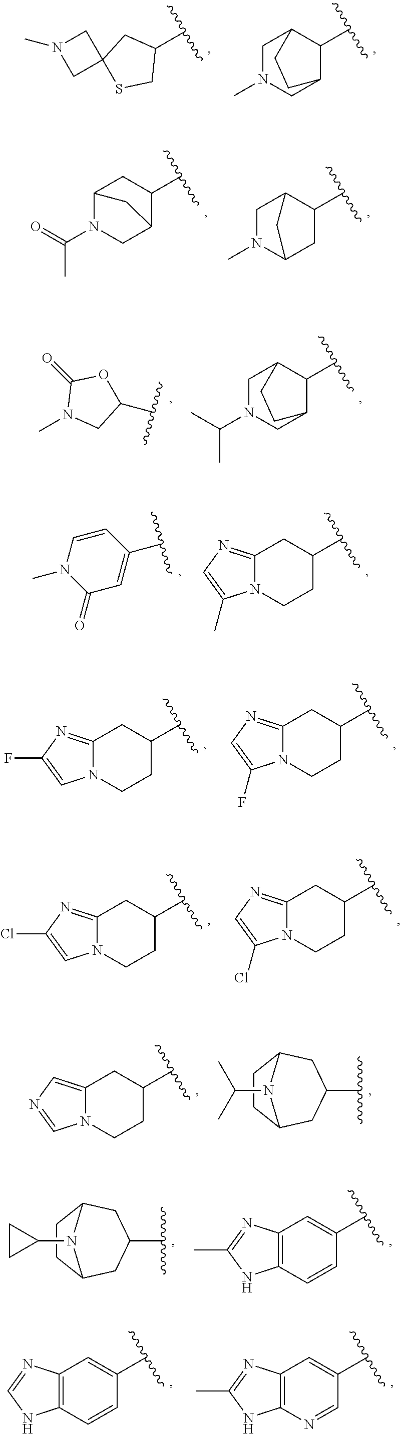

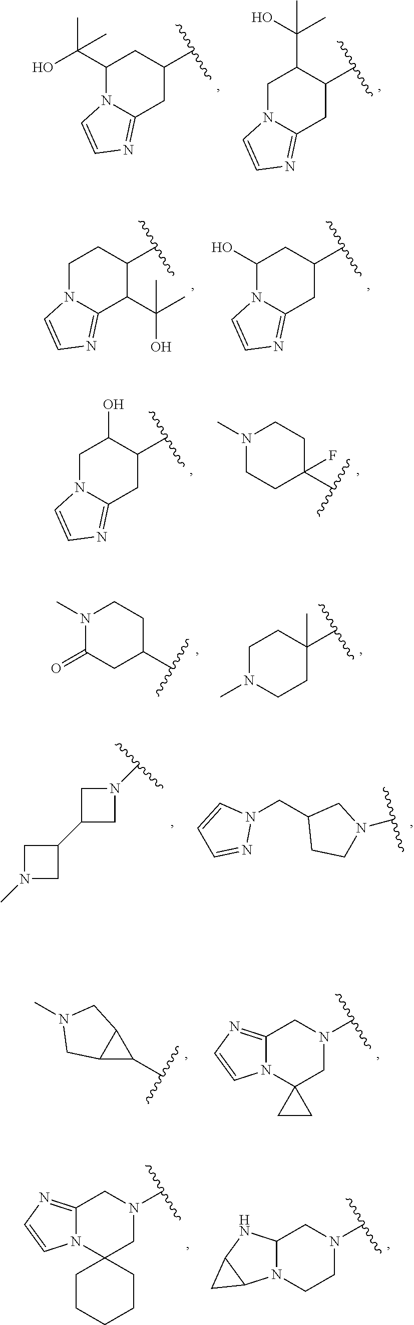

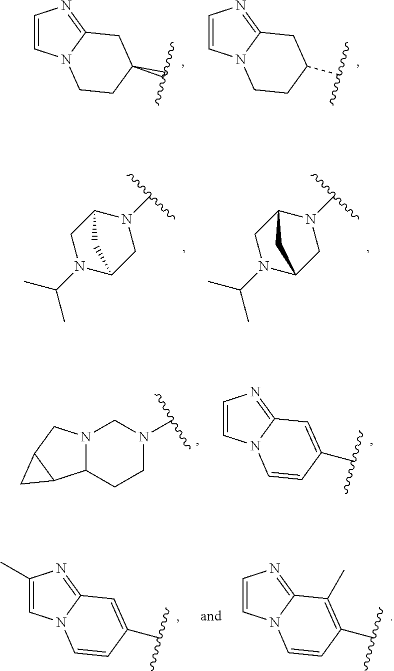

A can be selected from:

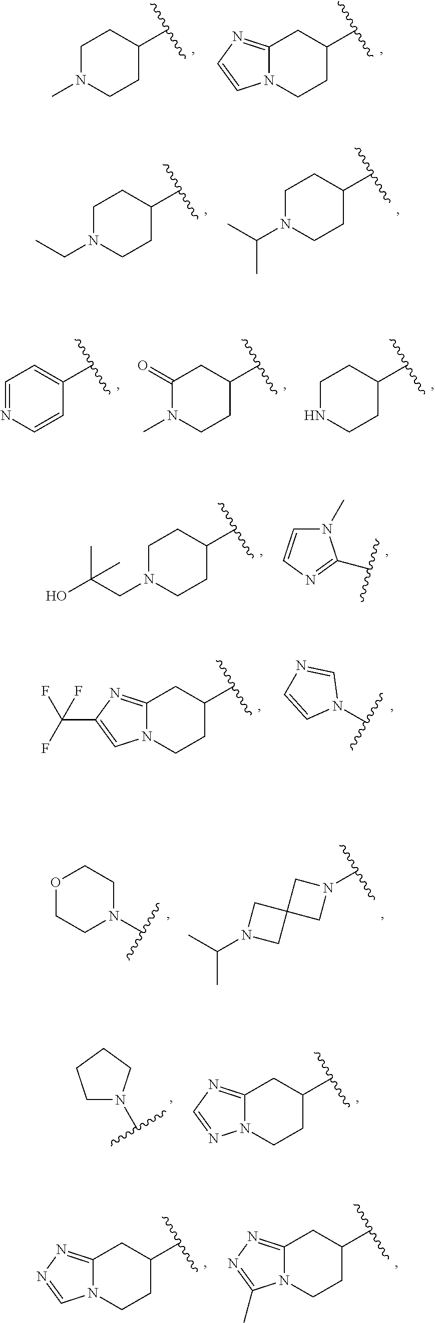

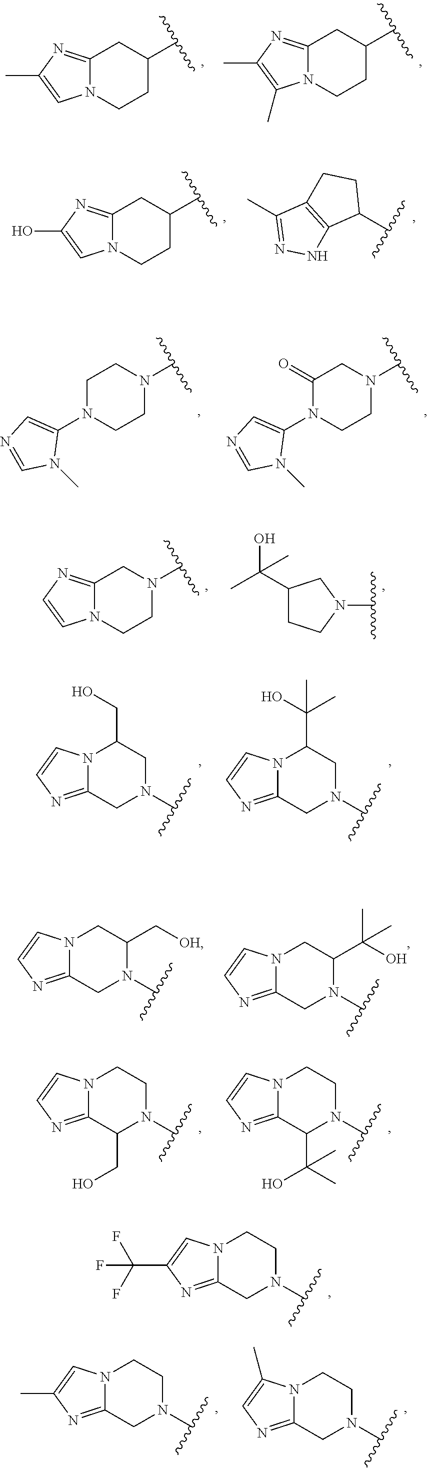

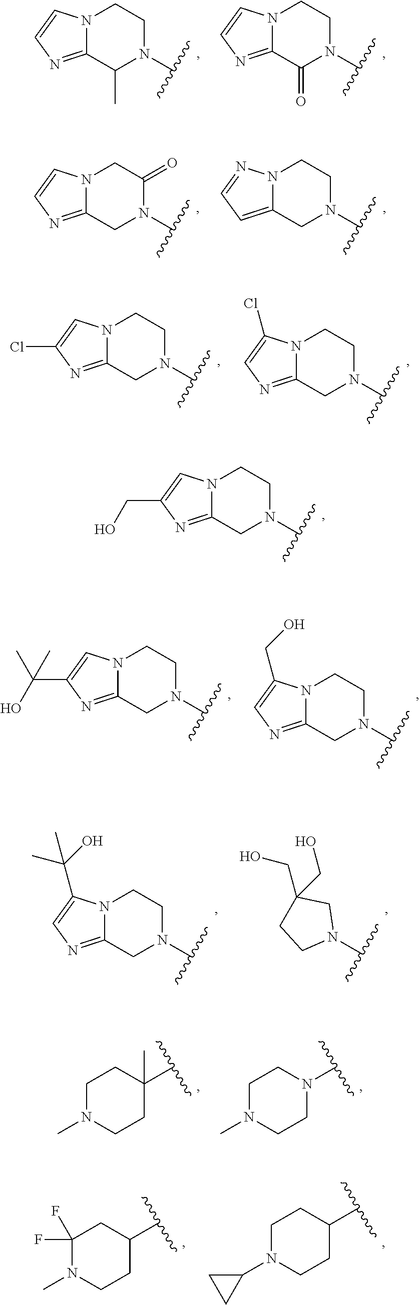

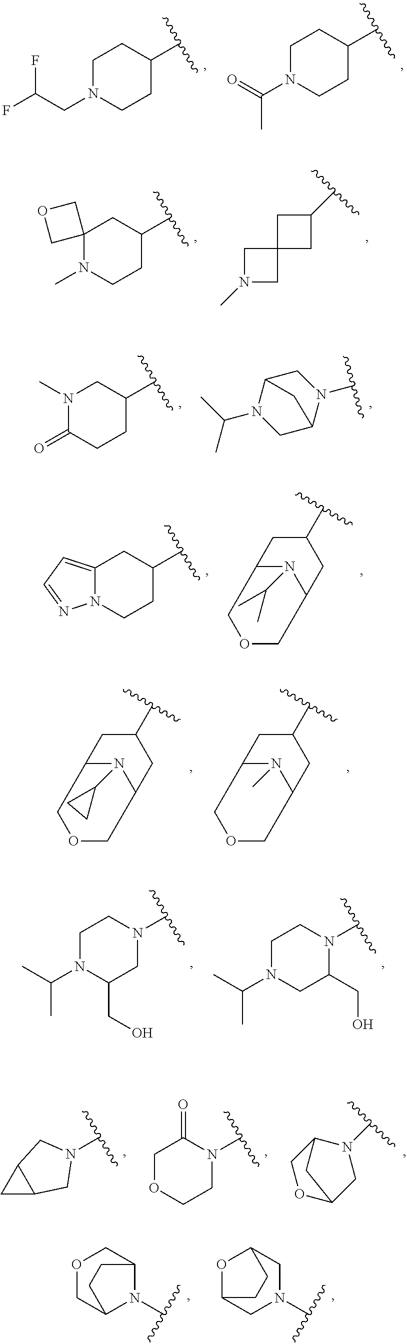

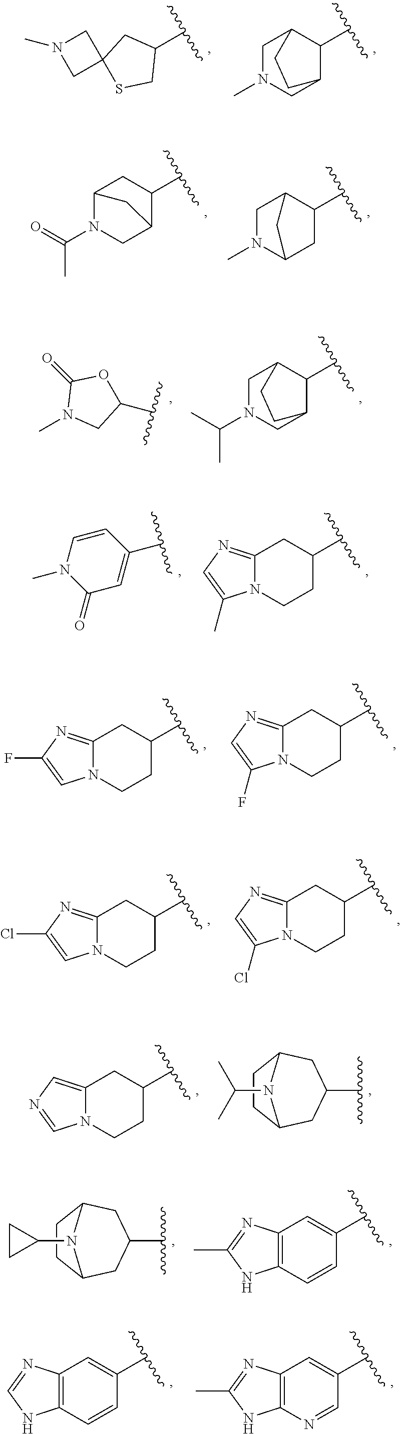

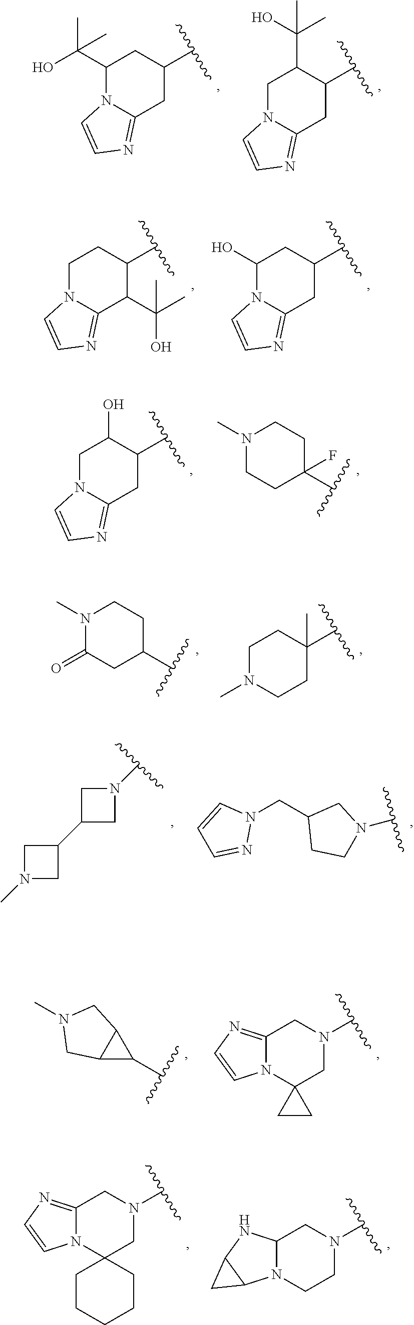

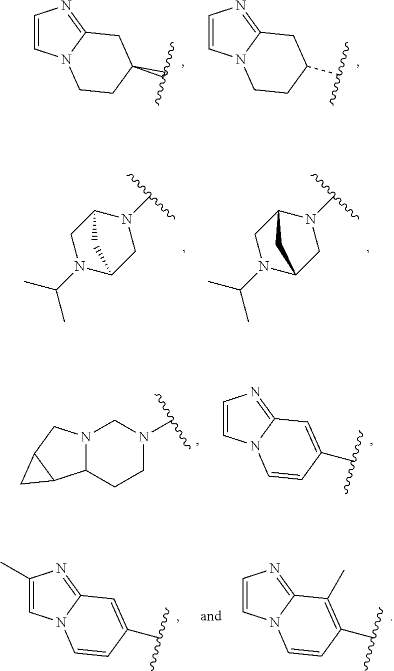

A can be selected from:

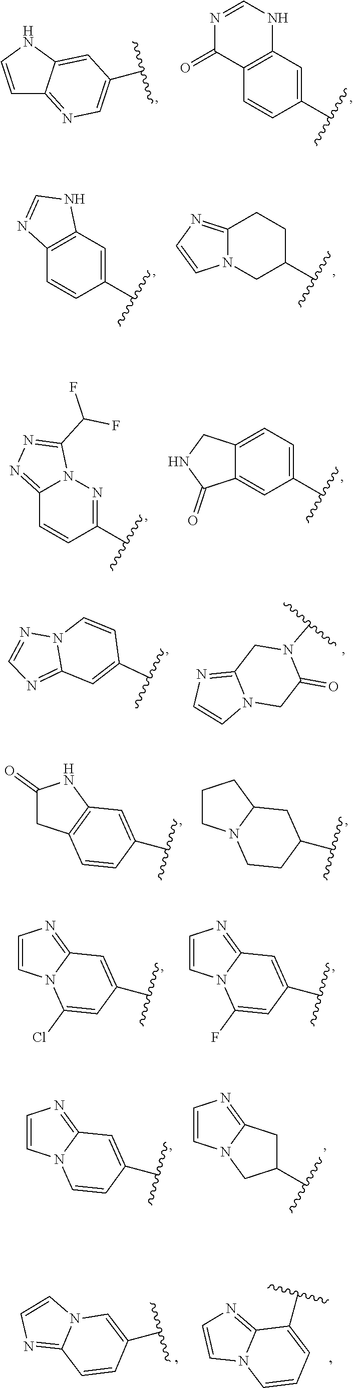

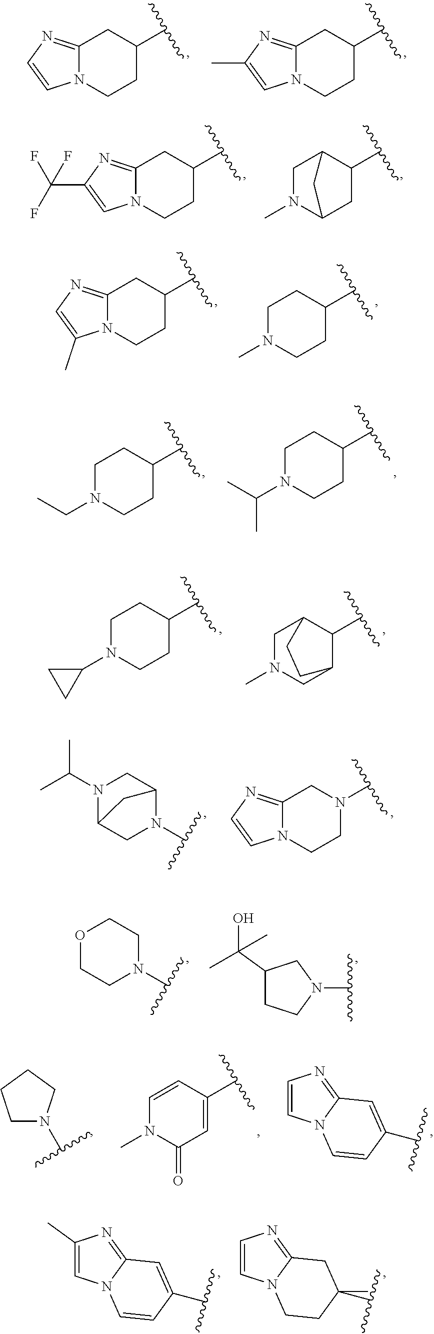

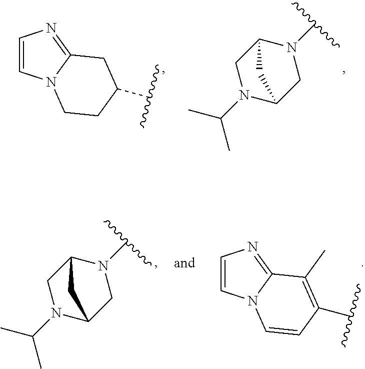

A can be selected from:

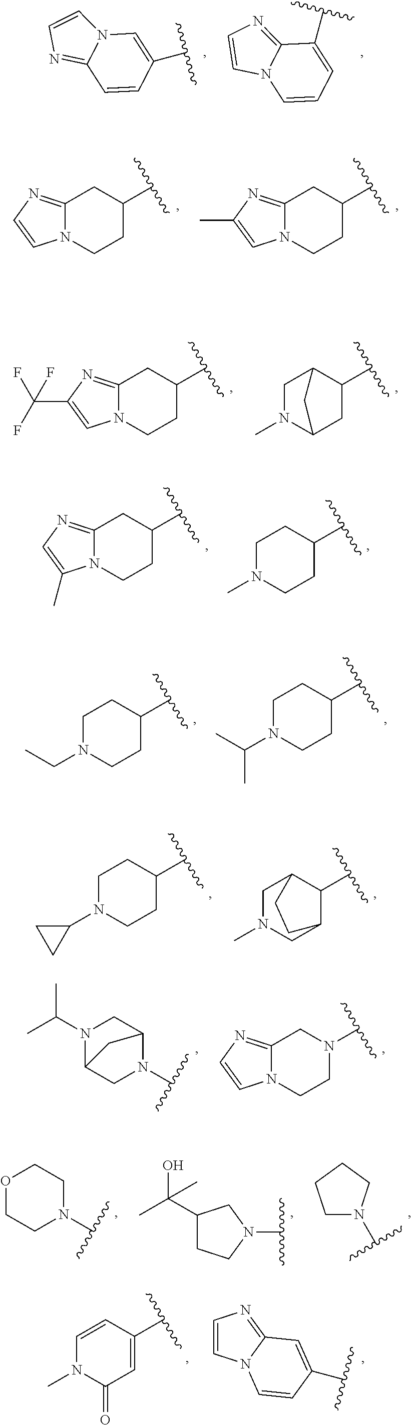

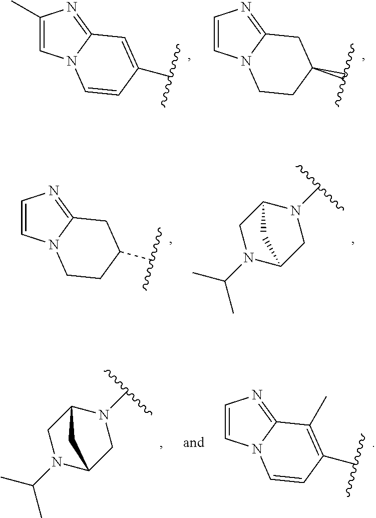

A can be selected from:

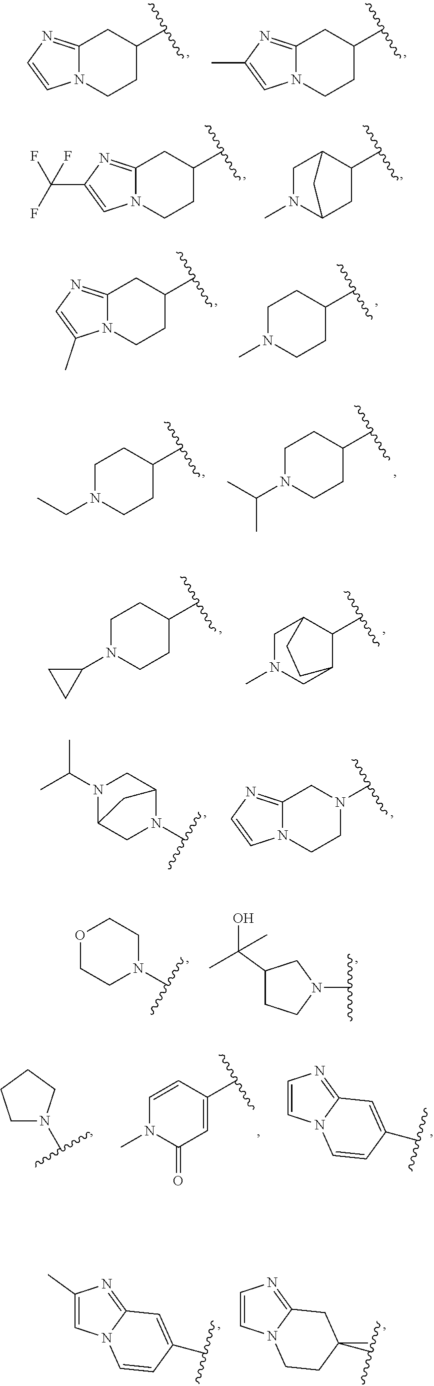

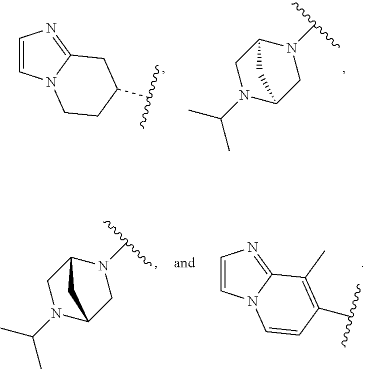

A can be selected from:

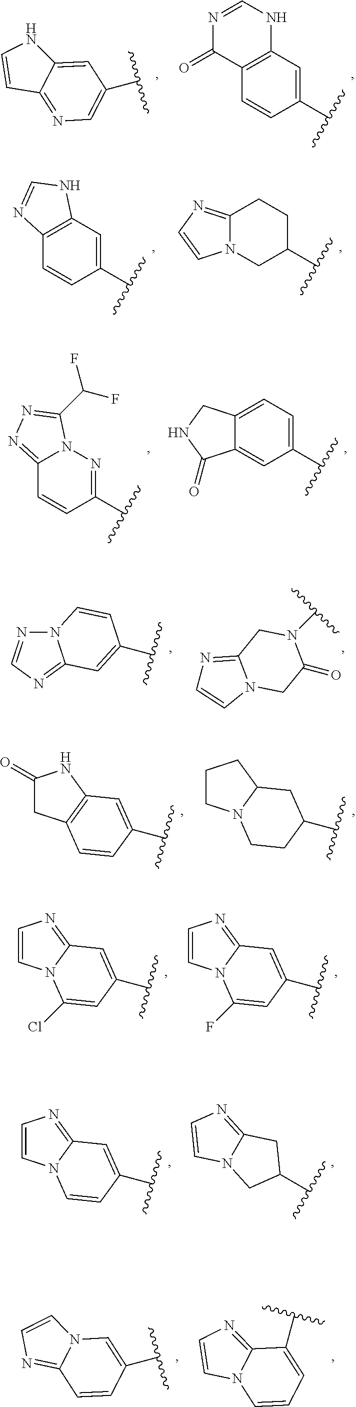

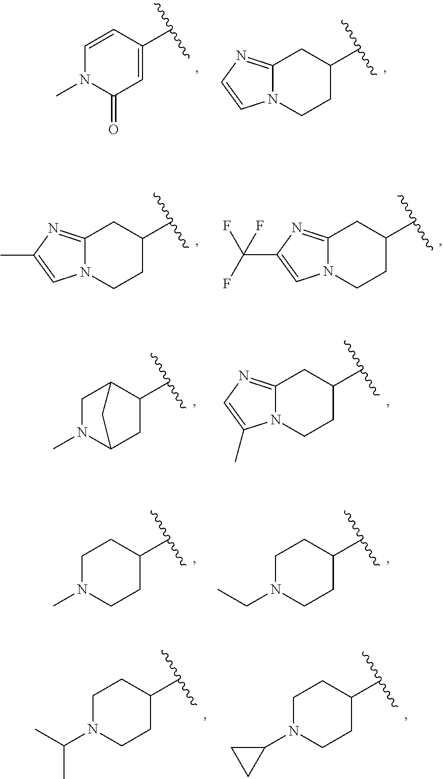

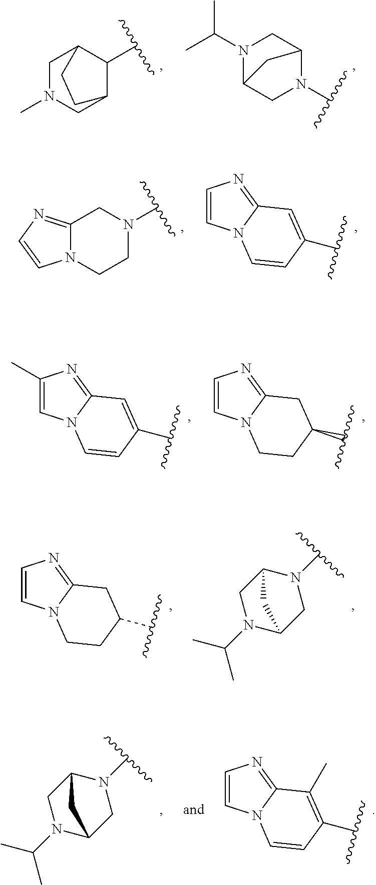

A can be selected from:

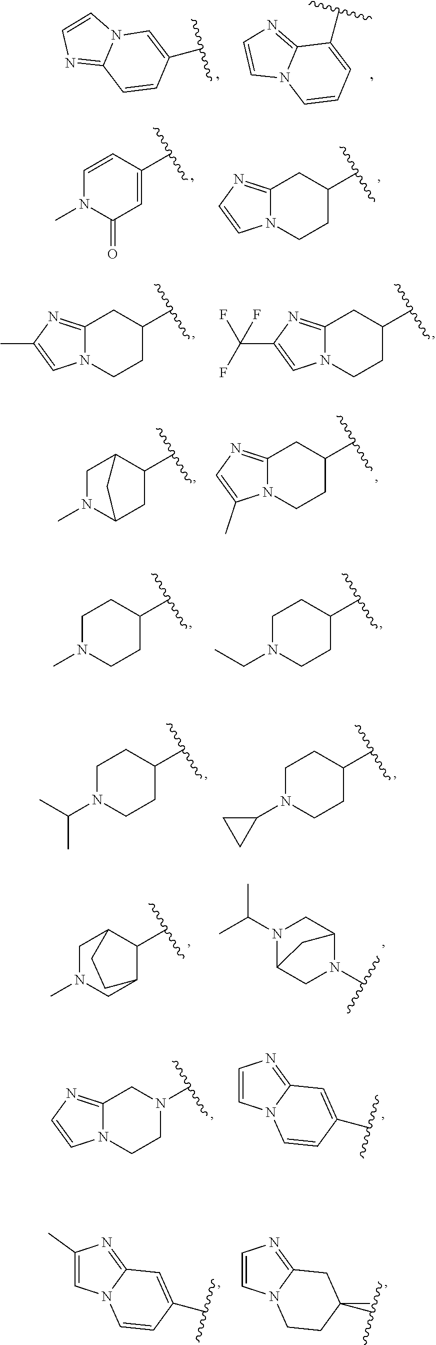

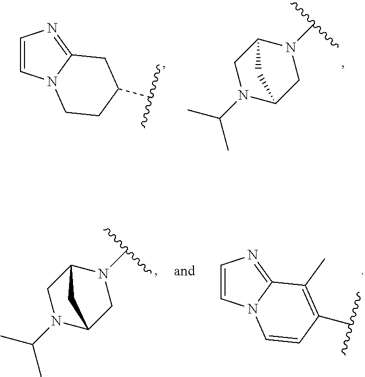

A can be selected from:

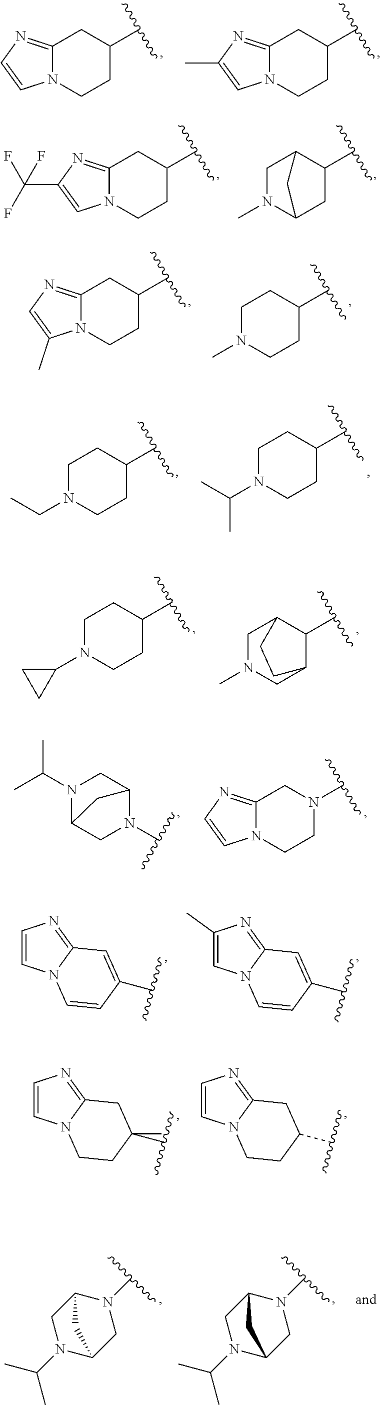

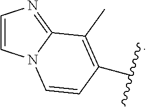 [SMILES: *c1ccn2ccnc2c1C]

Preferably, A is selected from:

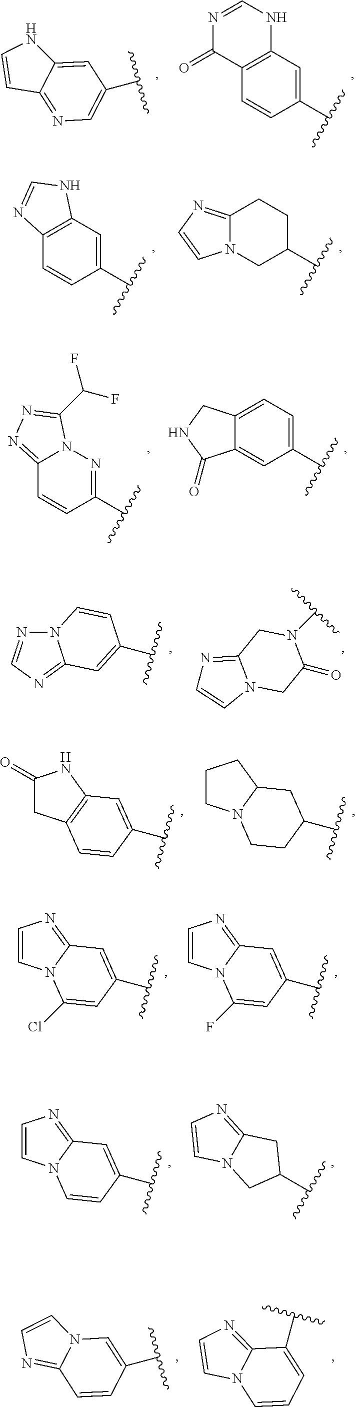

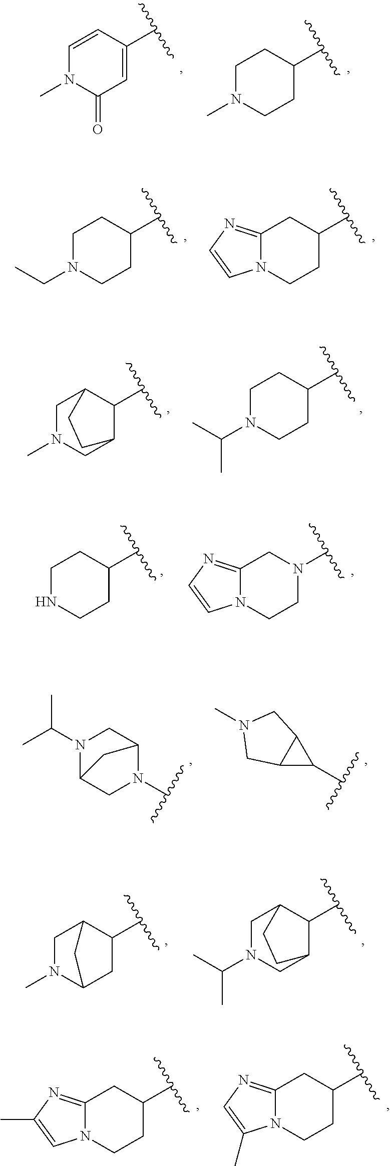

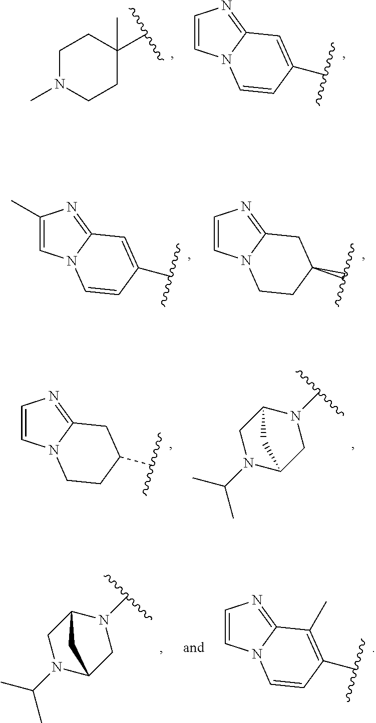

Preferably, A is selected from:

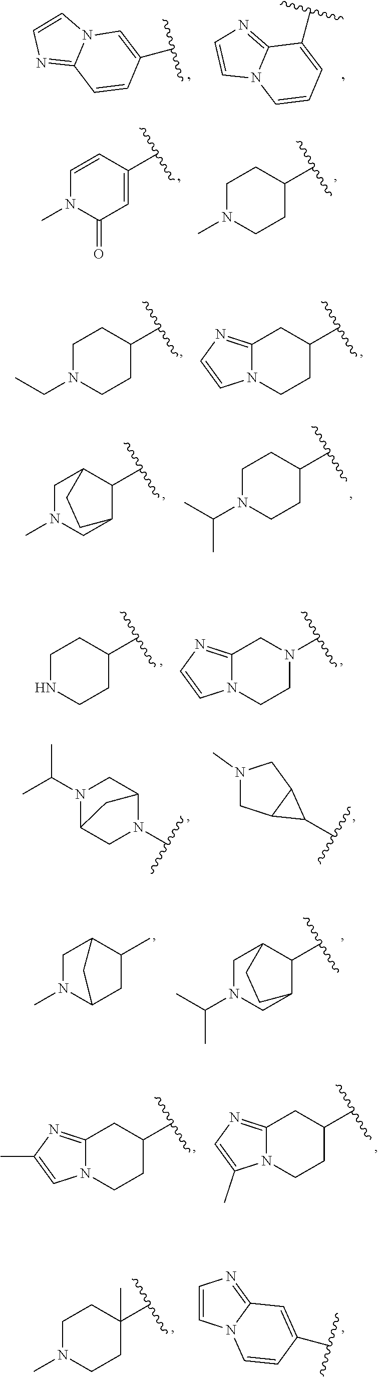

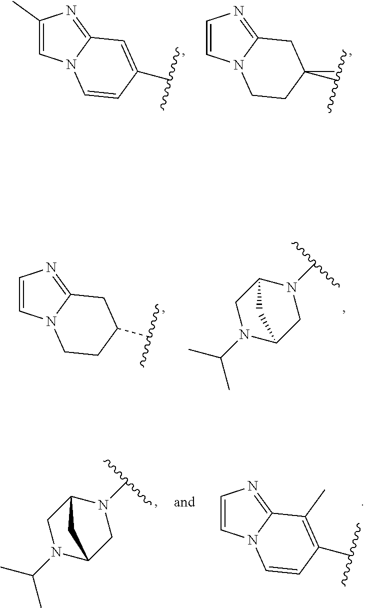

Preferably, A is selected from:

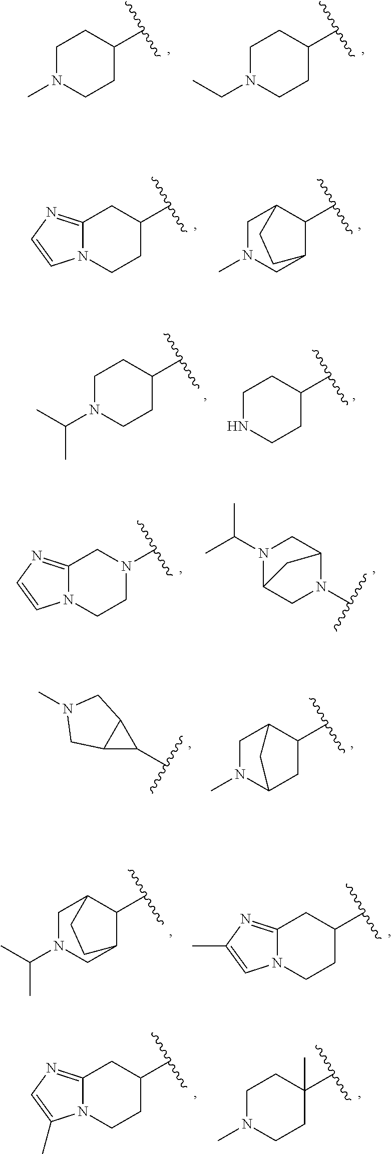

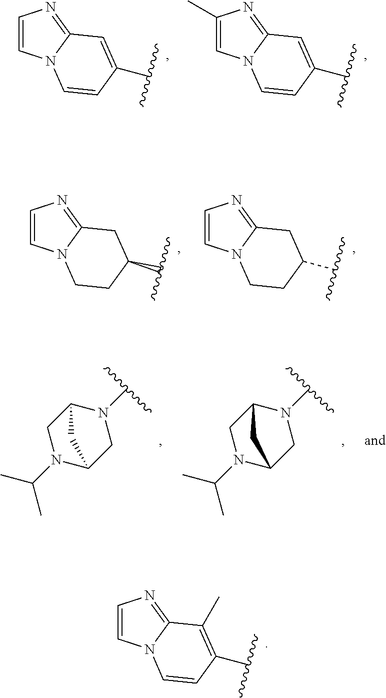

Preferably, A is selected from:

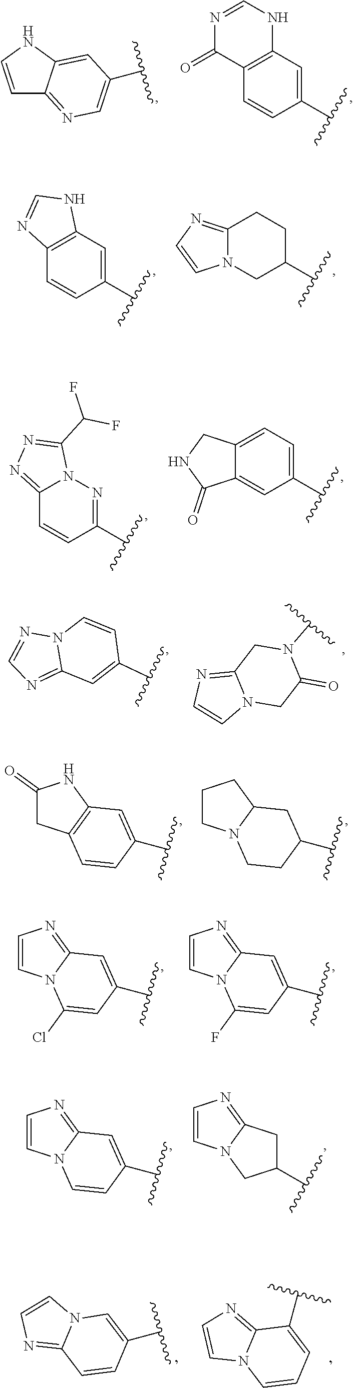

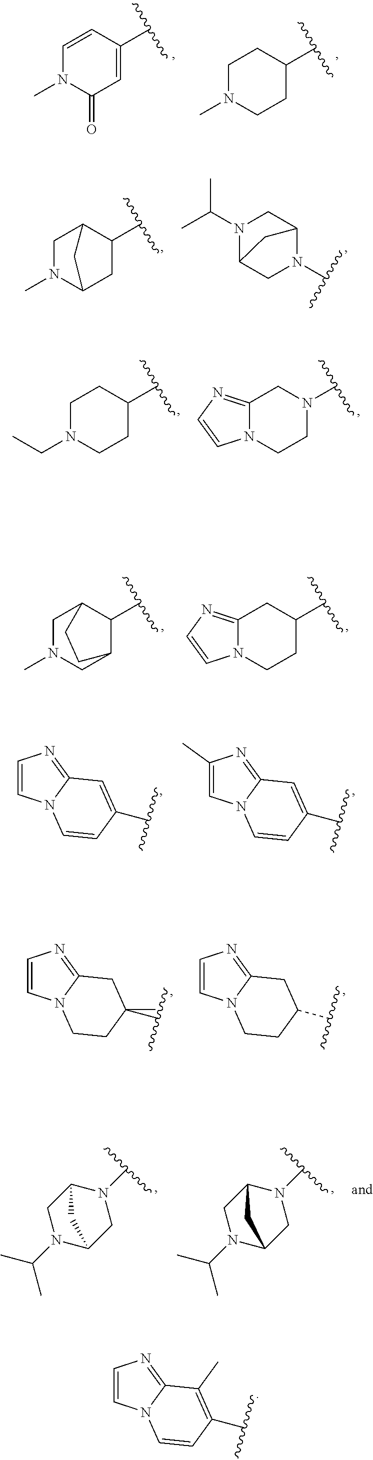

Preferably, A is selected from:

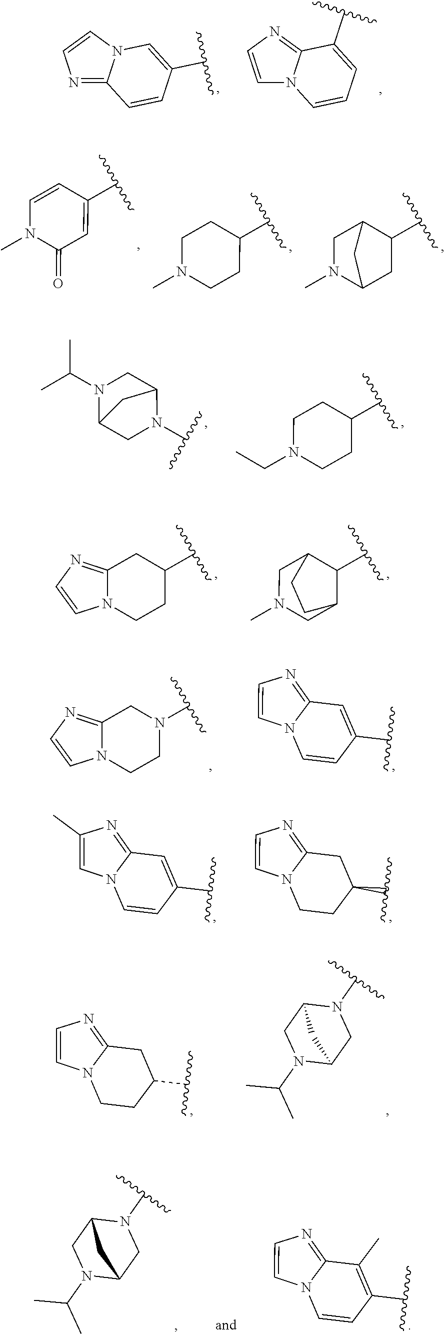

Preferably, A is selected from:

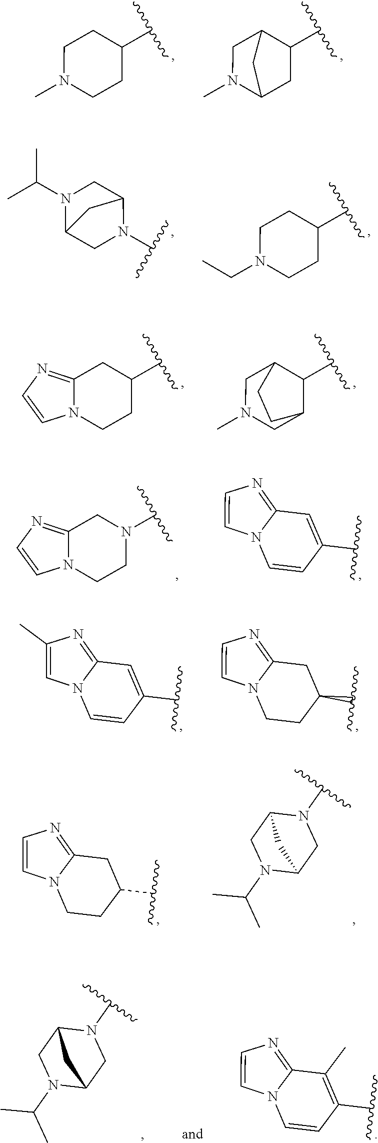

More preferably, A is selected from:

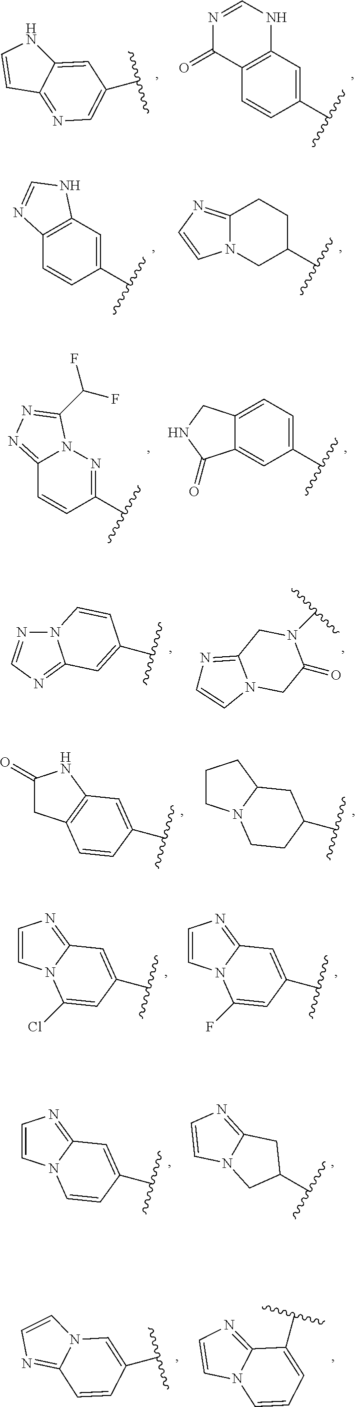

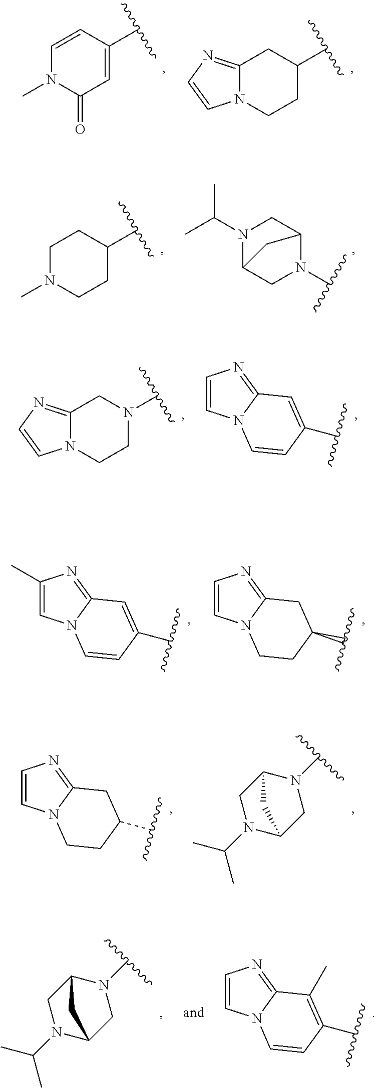

More preferably, A is selected from:

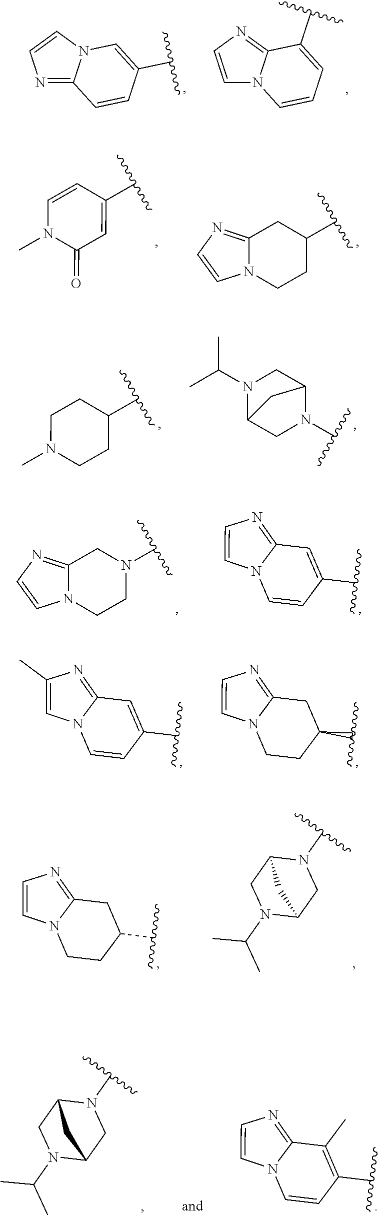

More preferably, A is selected from:

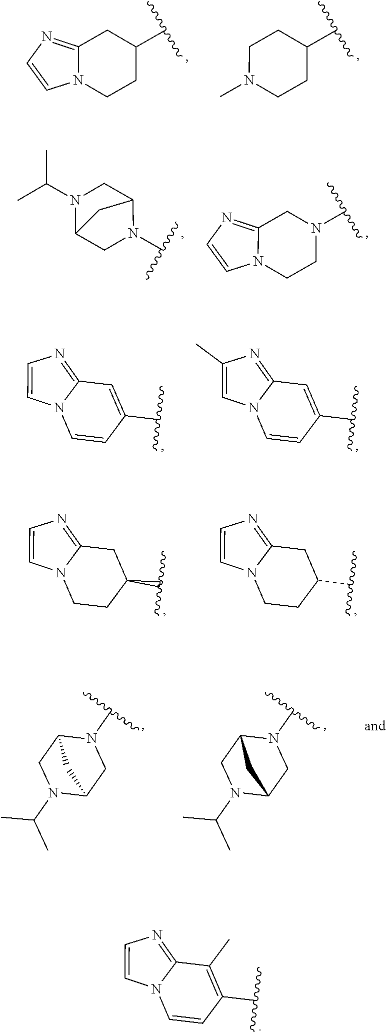

More preferably, A is selected from:

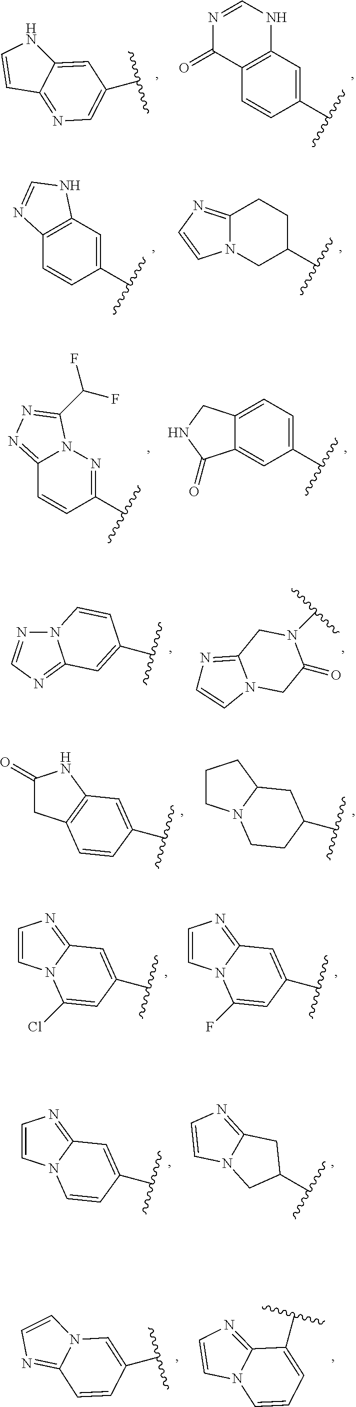

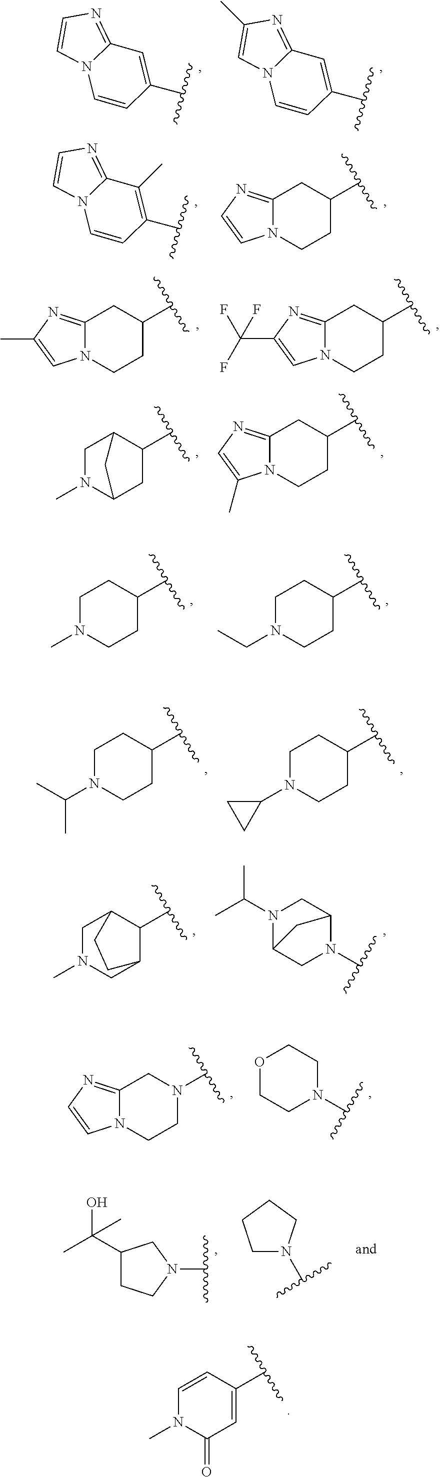

More preferably, A is selected from:

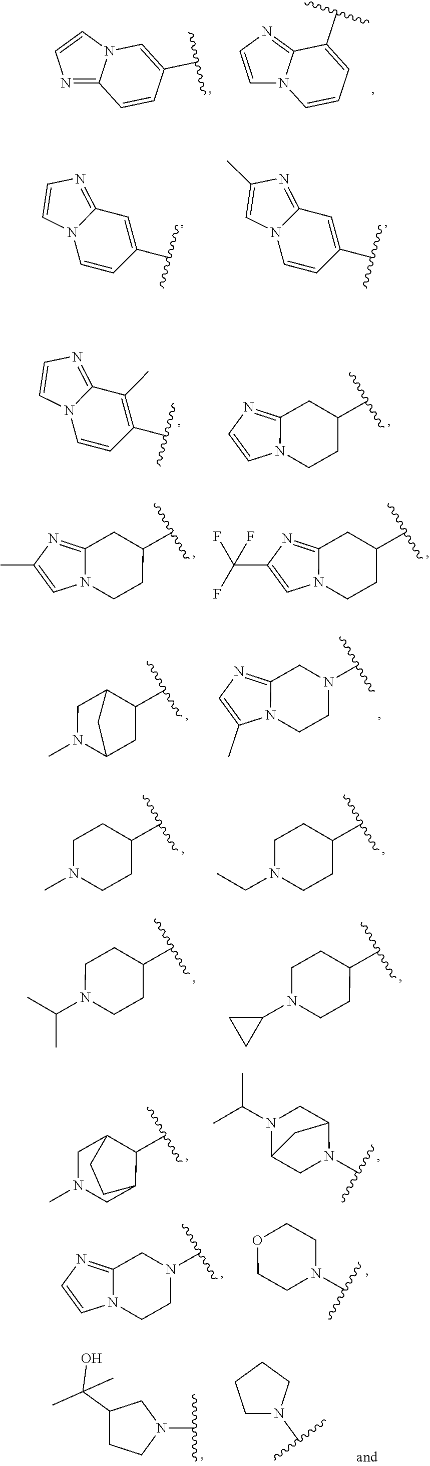

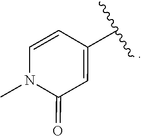 [SMILES: *c1ccn(C)c(=O)c1]

More preferably, A is selected from:

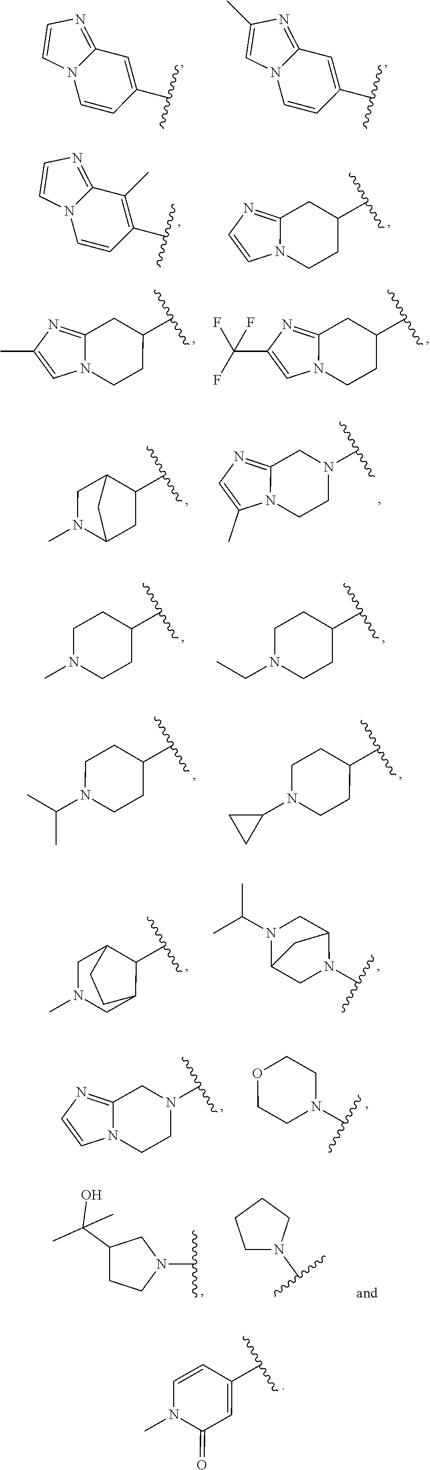

Most preferably, A is selected from:

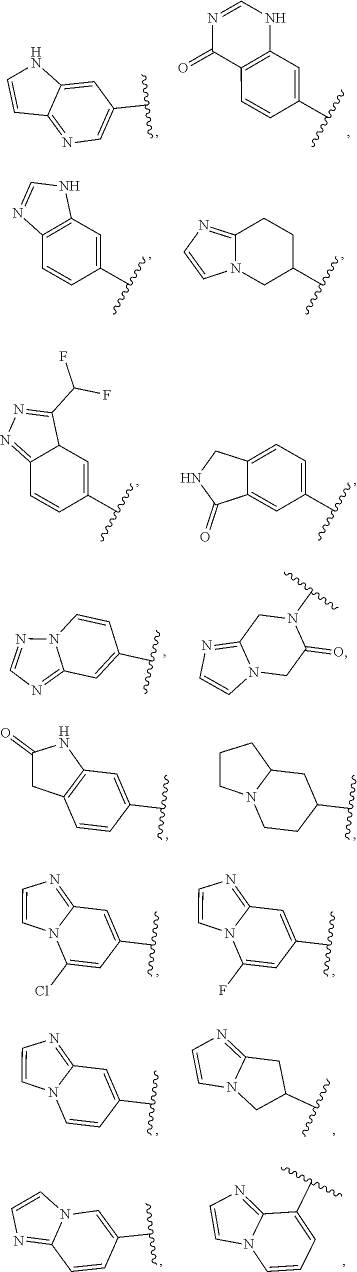

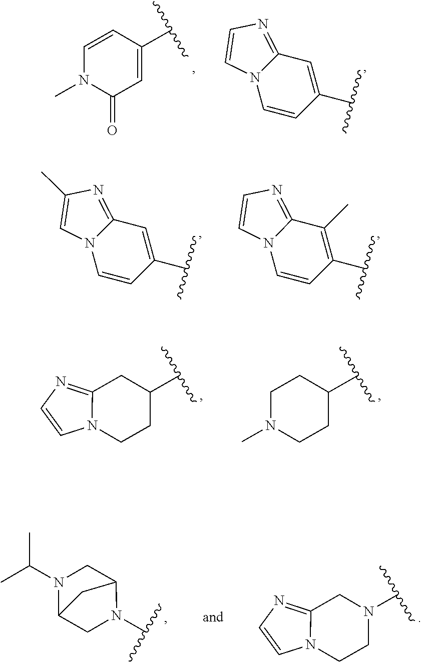

Most preferably, A is selected from:

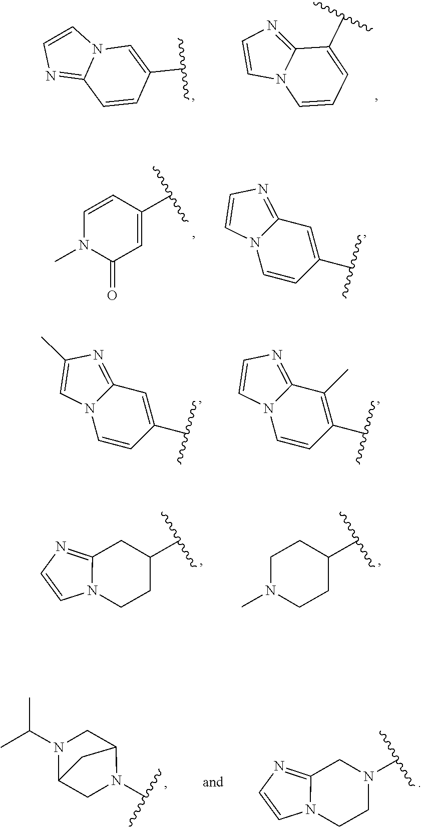

Most preferably, A is selected from:

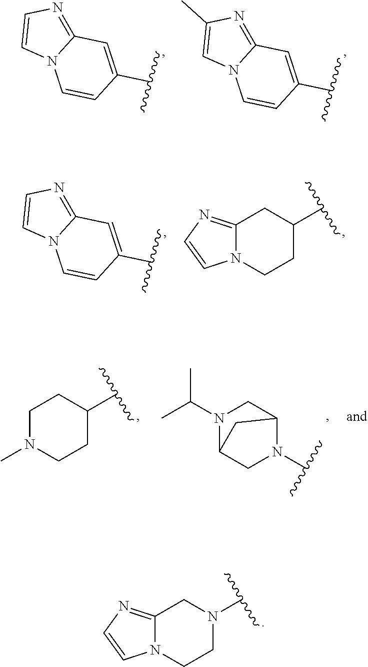

Even more preferably, A is selected from:

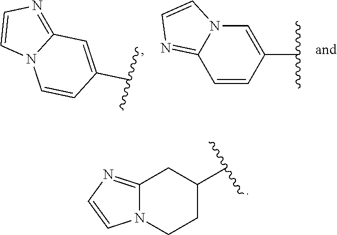 [SMILES: *c1ccn2ccnc2c1 ; *c1ccc2nccn2c1 ; *C1CCn2ccnc2C1]

Alternatively, A is selected from:

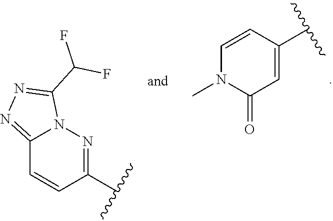

Specifically, A can be

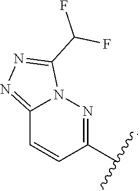 [SMILES: *c1ccc2nnc(C(F)F)n2n1]

Specifically, A can be

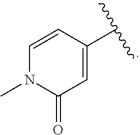 [SMILES: *c1ccn(C)c(=O)c1]

Preferably, A is not:
(i)

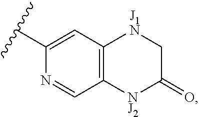

which may be optionally substituted at J₁, J₂, or any other ring position on A; or
(ii)

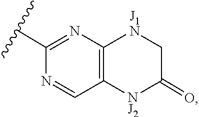

which may be optionally substituted at J₁, J₂, or any other ring position on A.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

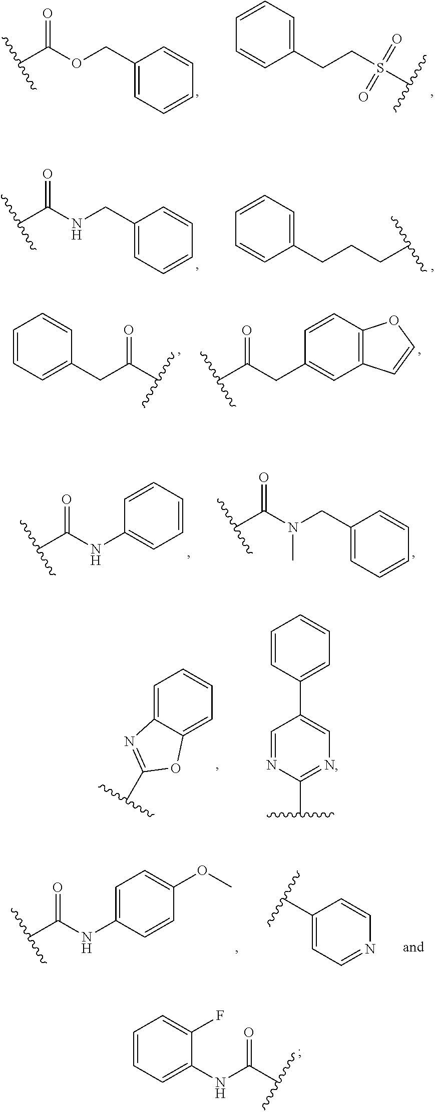

AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro.

Preferably, -V-Z- is selected from —CH₂—, —O—CH₂—, —CH₂—O— and —CH₂—CH₂—O—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro.

Preferably, AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro.

More specifically, when not absent, -V-Z- is selected from —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro.

More specifically, when not absent, -V-Z- is selected from —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:

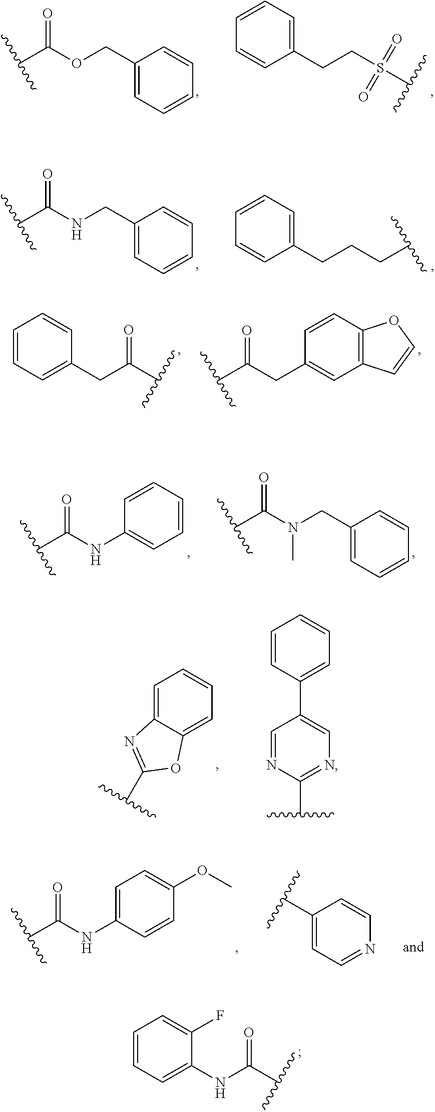

AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro.

More specifically, -V-Z- is selected from —CH₂—, —O—CH₂—, —CH₂—O— and —CH₂—CH₂—O—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A;

and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro.

More specifically, AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro.

Alternatively, when not absent, -V-Z- is selected from:
-V-Z- is selected from: —CH₂—, —O—CH₂—, —O—C(CH₃)₂—, —CH₂—O—,
—C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl.

Alternatively, when not absent, -V-Z- is selected from:
-V-Z- is selected from: —CH₂—, —O—CH₂—, —O—C(CH₃)₂—, —CH₂—O—,
—C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A;
and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, CH₂—CH₂—O—, and —CH₂—CH₂—CH₂—; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A;
and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A;
and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

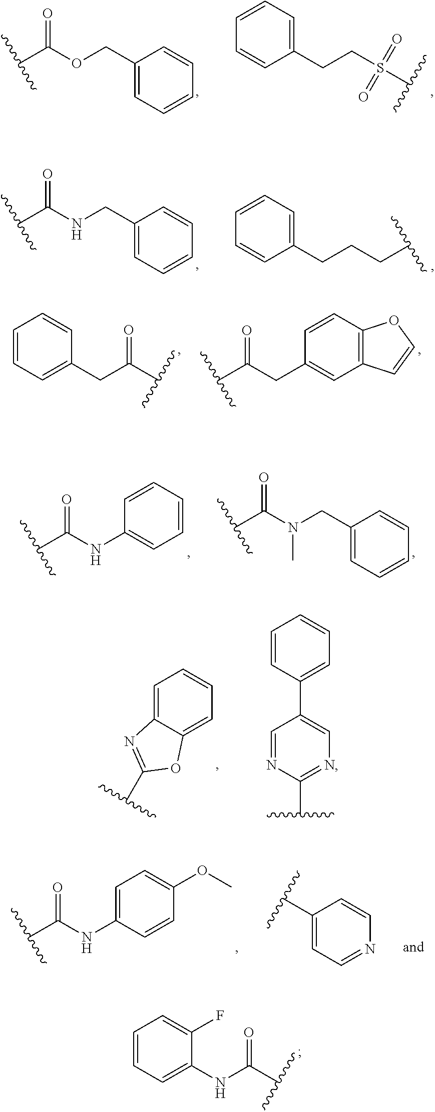

AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A;
and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl.

Alternatively, -V-Z- is selected from —CH₂—, —O—CH₂—, —CH₂—O— and —CH₂—CH₂—O—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A;
and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl.

Alternatively, AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl.

More preferably, AW- is selected from:
—(CHR12)₀₋₆-A (e.g. —(CHR12)₀₋₃-A, specifically —(CH₂)-A) and —(CH₂)₀₋₆—O—(CH₂)₀₋₆-A (e.g. —(CH₂)₀₋₃—O—(CH₂)_{0- 3}-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

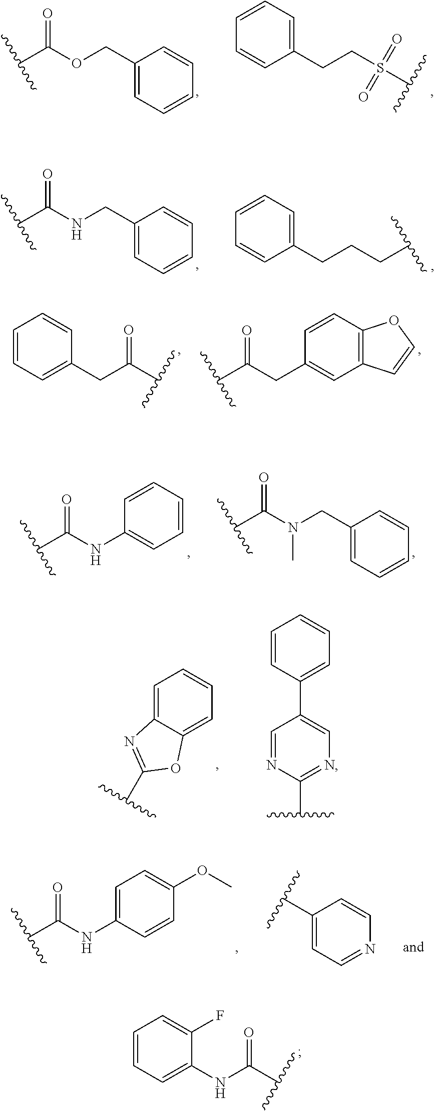

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

More preferably, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently C(═O)OR13, C(═O)NR13R14, NR13R14, CF₃, CN; wherein one of the rings in the bicyclic ring system can be aromatic or both of the rings in the bicyclic ring system can be aromatic.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

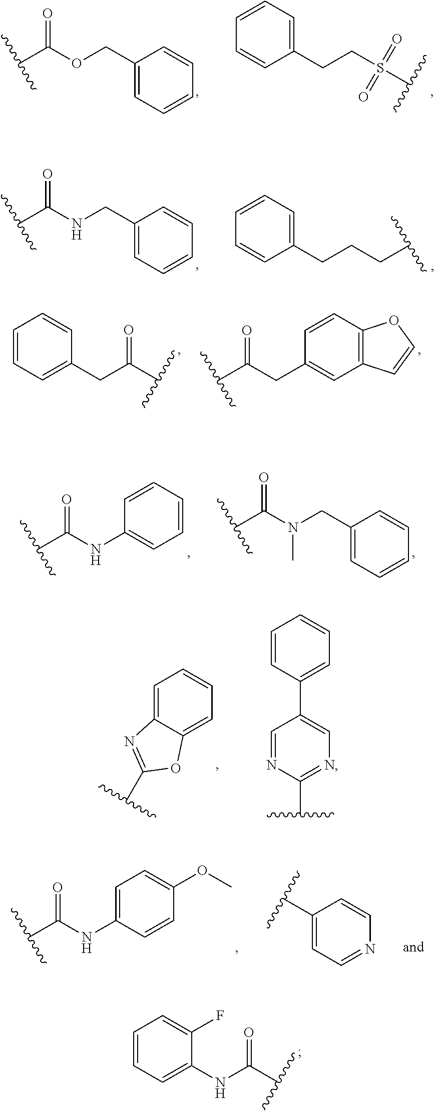

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12,
C(═O)OR13, C(═O)NR13R14, NR13R14, CF₃, CN; wherein one of the rings in the bicyclic ring system can be aromatic or both of the rings in the bicyclic ring system can be aromatic.

More preferably, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently C(═O)OR13, C(═O)NR13R14, NR13R14, CF₃, CN; wherein one of the rings in the bicyclic ring system can be aromatic or both of the rings in the bicyclic ring system can be aromatic.

More preferably, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, -V-Z- is selected from from —CH₂—, —O—CH₂—, —CH₂—O—, —CH₂—CH₂—O—, —CH₂—N(R18)- and —N(R18)-CH₂—, wherein R18 is selected from:

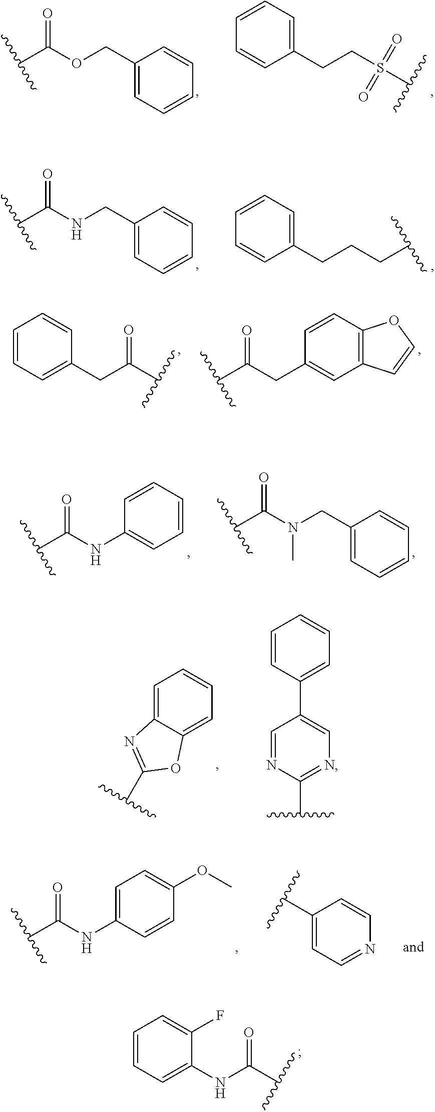

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, -V-Z- is selected from from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, -V-Z- is selected from from —CH₂—, —O—CH₂—, —CH₂—O—, —CH₂—CH₂—O—, —CH₂—N(R18)- and —N(R18)-CH₂—, wherein R18 is selected from:

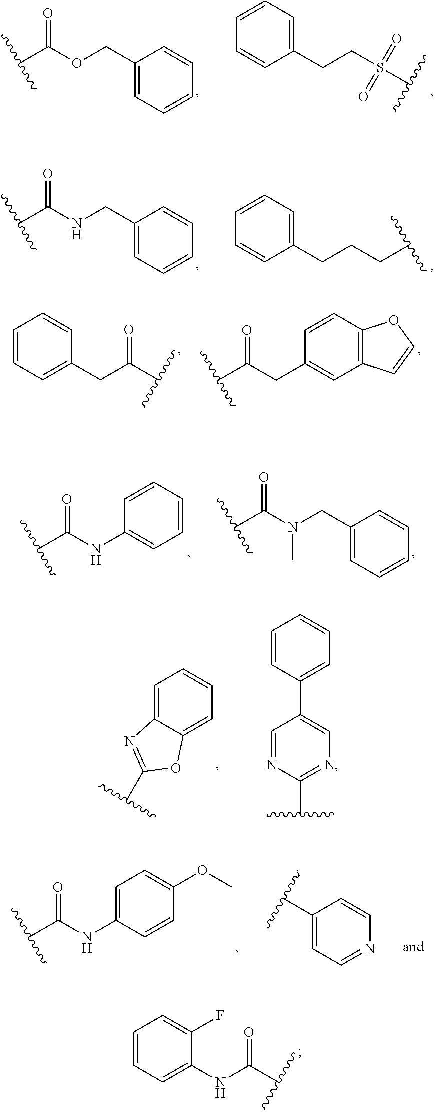

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; and X is CH₂ and Y is NH or N.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from: (CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; and X is CH₂ and Y is NH or N.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; and X is CH₂ and Y is NH or N.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; and X is CH₂ and Y is NH.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; and X is CH₂ and Y is NH.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

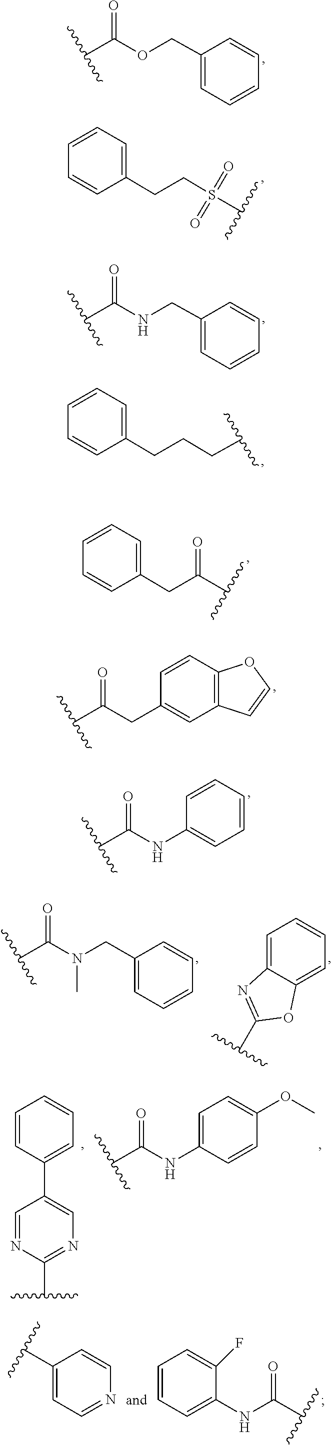

AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; and X is CH₂ and Y is NH.

Preferably, -V-Z- is selected from —CH₂—, —O—CH₂—, —CH₂—O— and —CH₂—CH₂—O—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; and X is CH₂ and Y is NH.

Preferably, AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; and X is CH₂ and Y is NH.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; and X is CH₂ and Y is NH or N.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; and X is CH₂ and Y is NH or N.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; and X is CH₂ and Y is NH or N.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; and X is CH₂ and Y is NH.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A;
and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; and X is CH₂ and Y is NH.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:

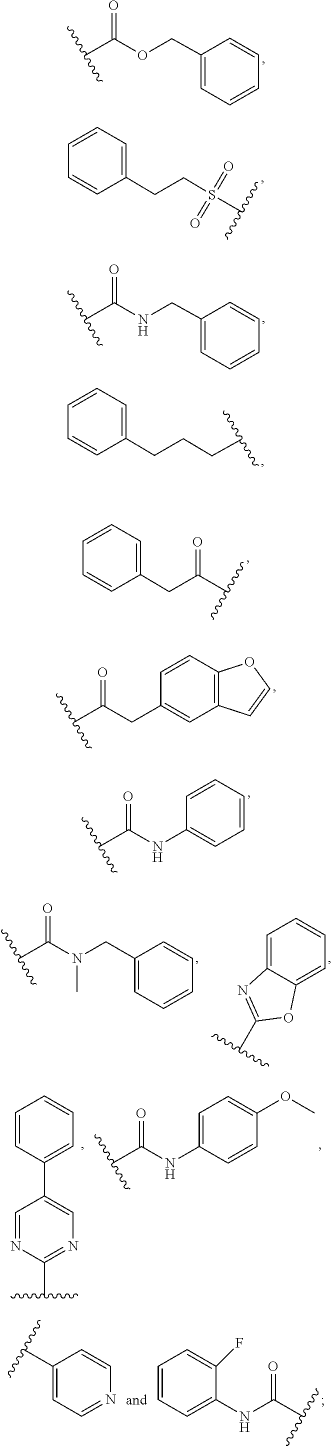

AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; and X is CH₂ and Y is NH.

More specifically, -V-Z- is selected from —CH₂—, —O—CH₂—, —CH₂—O— and —CH₂—CH₂—O—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; and X is CH₂ and Y is NH.

More specifically, AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A;
and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; and X is CH₂ and Y is NH.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH or N.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A;
and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH or N.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH or N.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH.

Alternatively, -V-Z- is selected-CH₂—, —O—CH₂—, —CH₂—O—, —CH₂—CH₂—O—, —CH₂—N(R18)- and —N(R18)-CH₂—, wherein R18 is selected from:

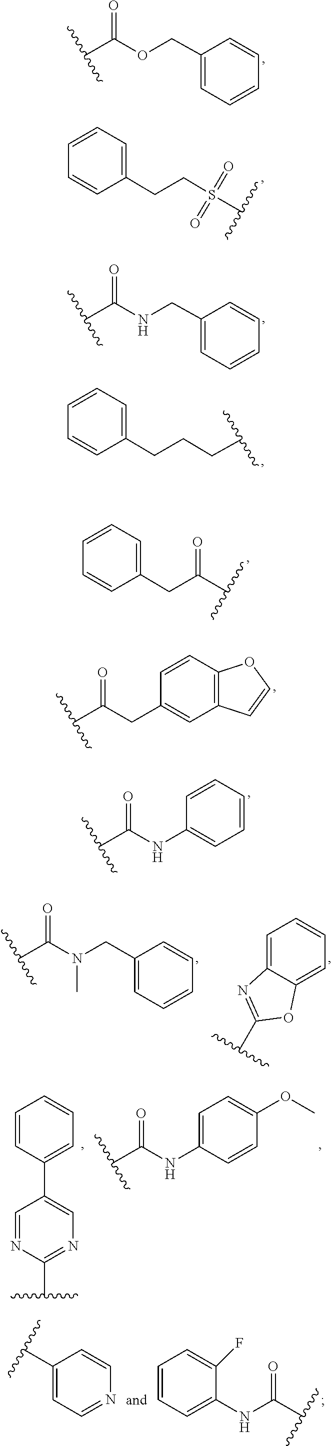

AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH.

Alternatively, -V-Z- is selected from —CH₂—, —O—CH₂—, —CH₂—O— and —CH₂—CH₂—O—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH.

Alternatively, AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH.

More preferably, AW- is selected from:
—(CHR12)₀₋₆-A (e.g. —(CHR12)₀₋₃-A, specifically —(CH₂)-A) and —(CH₂)₀₋₆—O—(CH₂)₀₋₆-A (e.g. —(CH₂)₀₋₃—O—(CH₂)_{0- 3}-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

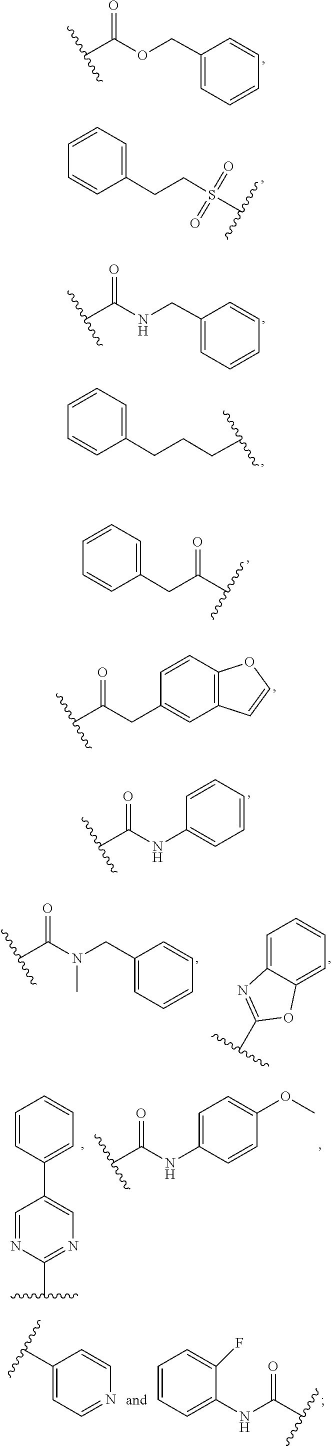

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12,
C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

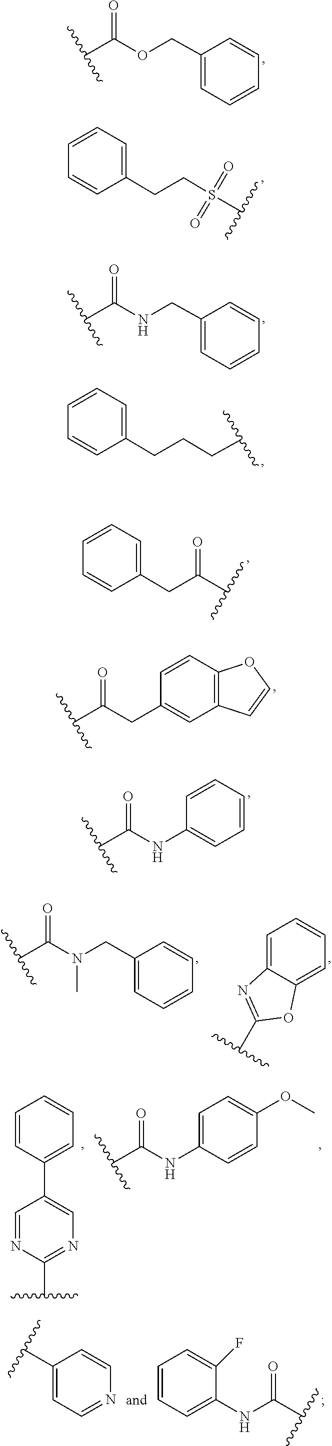

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is NH.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

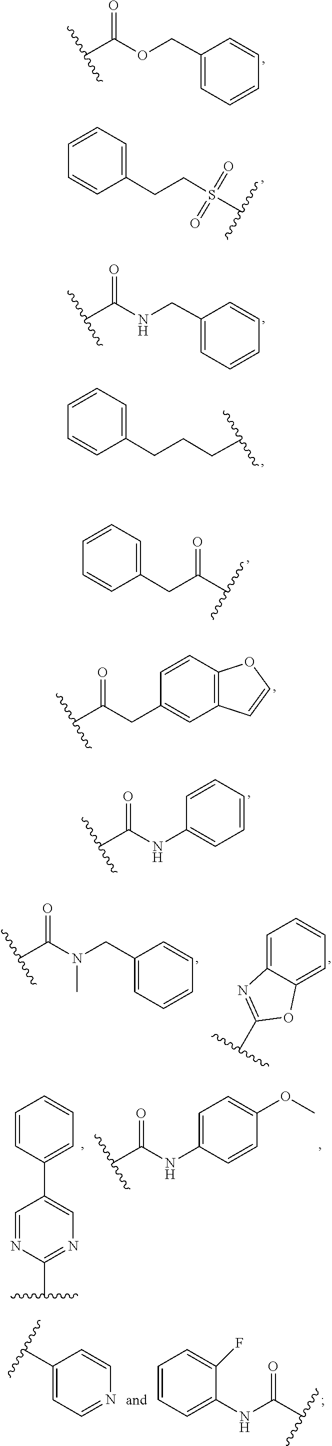

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12,
C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

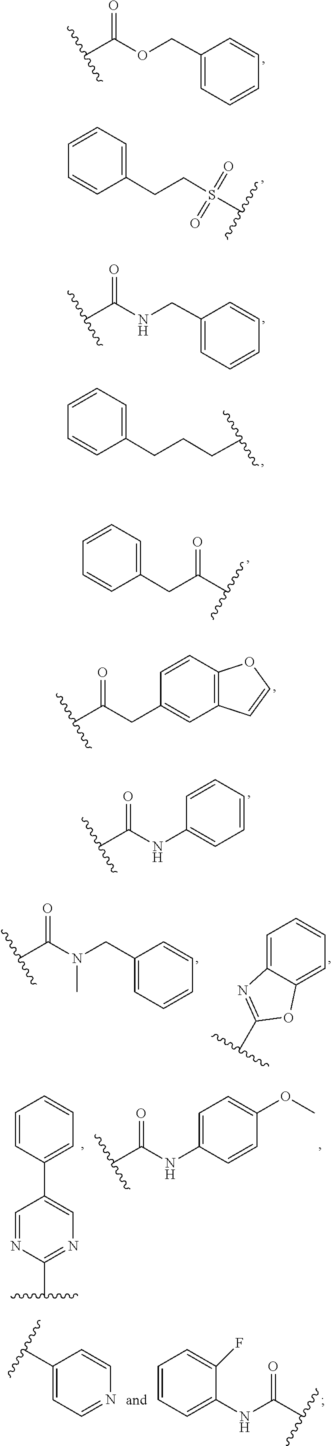

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

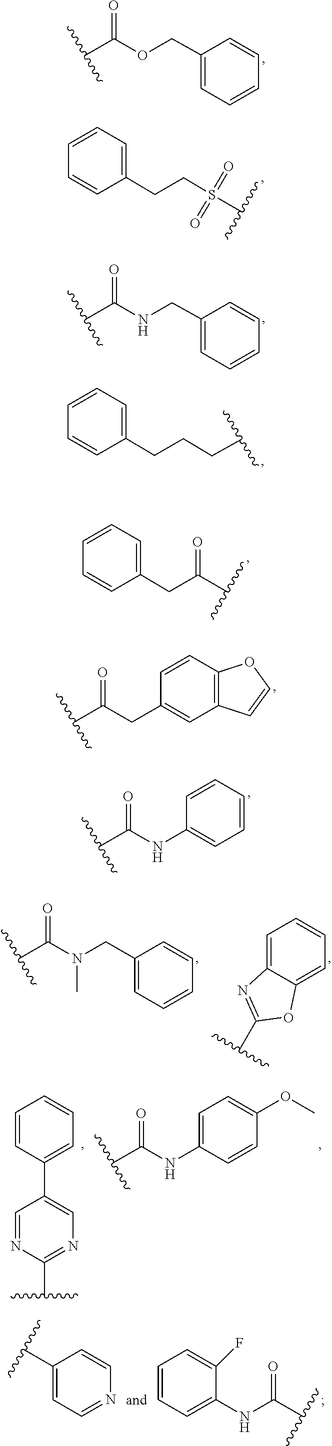

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH

More preferably, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH.

More preferably, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

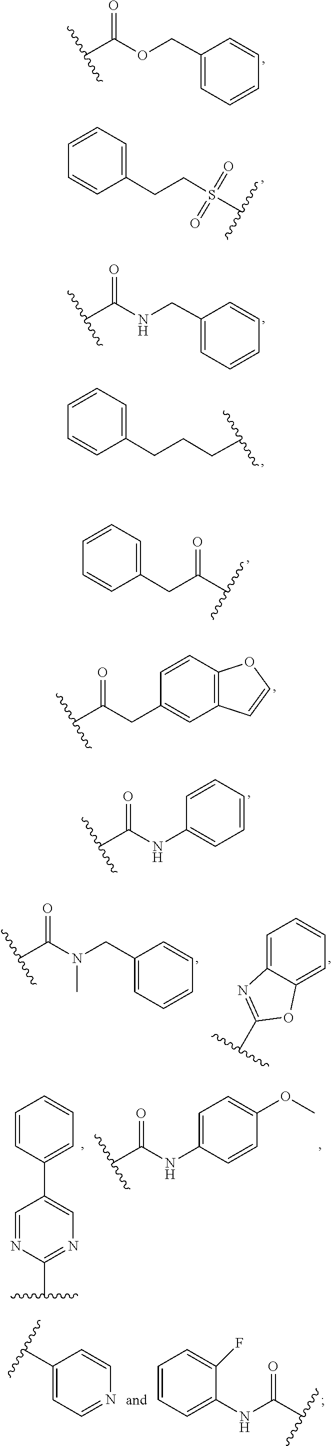

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH.

More preferably, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH.

More preferably, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

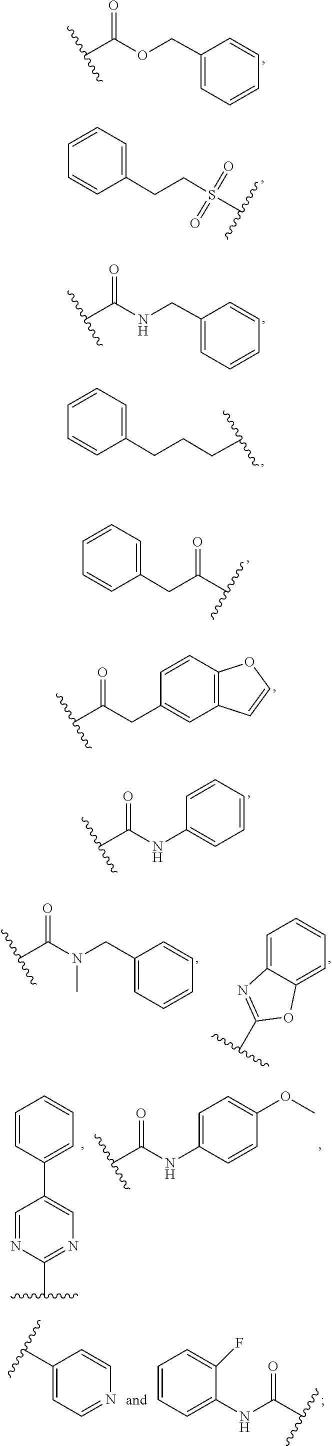

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13,
C(═O)NR13R14, NR13R14, CF₃, CN; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

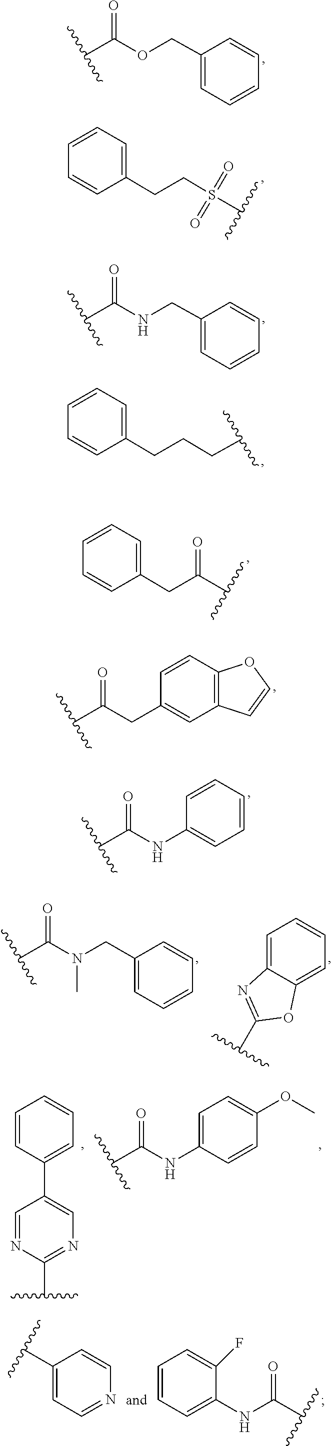

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

More preferably, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is O.

—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; X is CH₂ and Y is NH or N; and B is heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; X is CH₂ and Y is NH or N; and B is heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; X is CH₂ and Y is NH or N; and B is heteroaryl^{a}.

Preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; X is CH₂ and Y is NH; and B is heteroaryl^{a}.

Preferably, -V-Z- is selected from CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; X is CH₂ and Y is NH; and B is heteroaryl^{a}.

Preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

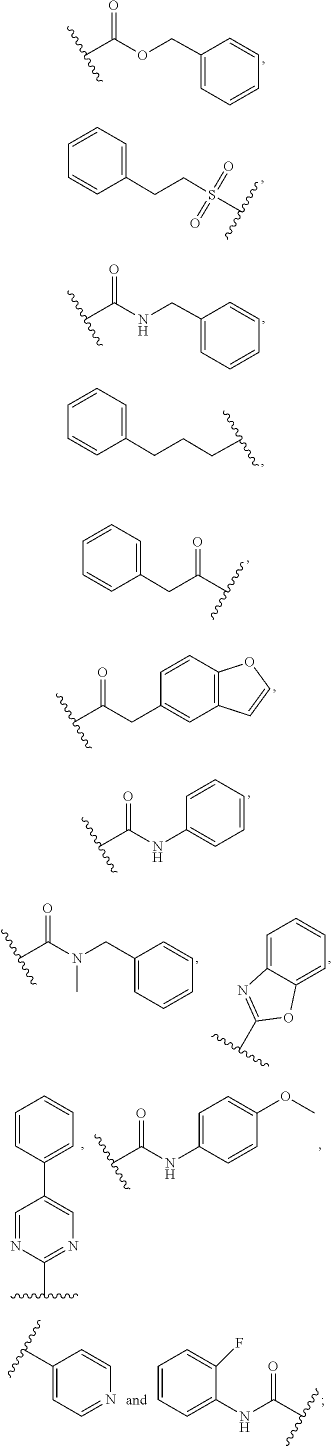

—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; X is CH₂ and Y is NH; and B is heteroaryl^{a}.

Preferably, -V-Z- is selected from —CH₂—, —O—CH₂—, —CH₂—O— and —CH₂—CH₂—O—;
AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; X is CH₂ and Y is NH; and B is heteroaryl^{a}.

Preferably, AW- is selected from:
—O—(CHR12)-A, —(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A, —(CH₂)₀₋₃—NR12-(CH₂)₁₋₃—C(═O)-A and —C(═O)NR12-(CH₂)₀₋₃-A; and A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein when A is a bicyclic ring system, the bicyclic ring system is fused, bridged or spiro; X is CH₂ and Y is NH; and B is heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; X is CH₂ and Y is NH or N; and B is heteroaryl^{a} and selected from isoquinolinyl and azaindole (specifically 7-azaindole), optionally substituted as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; X is CH₂ and Y is NH or N; and B is heteroaryl^{a} and selected from isoquinolinyl and azaindole (specifically 7-azaindole), optionally substituted as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; X is CH₂ and Y is NH or N;
and B is heteroaryl^{a} and selected from isoquinolinyl and azaindole (specifically 7-azaindole), optionally substituted as for heteroaryl^{a}.

More specifically, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; X is CH₂ and Y is NH; and B is heteroaryl^{a} and selected from isoquinolinyl and azaindole (specifically 7-azaindole), optionally substituted as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; X is CH₂ and Y is NH; and B is heteroaryl^{a} and selected from isoquinolinyl and azaindole (specifically 7-azaindole), optionally substituted as for heteroaryl^{a}.

More specifically, -V-Z- is selected from —CH₂—, —O—CH₂—, —CH₂—O—, —CH₂—CH₂—O—, —CH₂—N(R18)- and —N(R18)-CH₂—, wherein R18 is selected from:

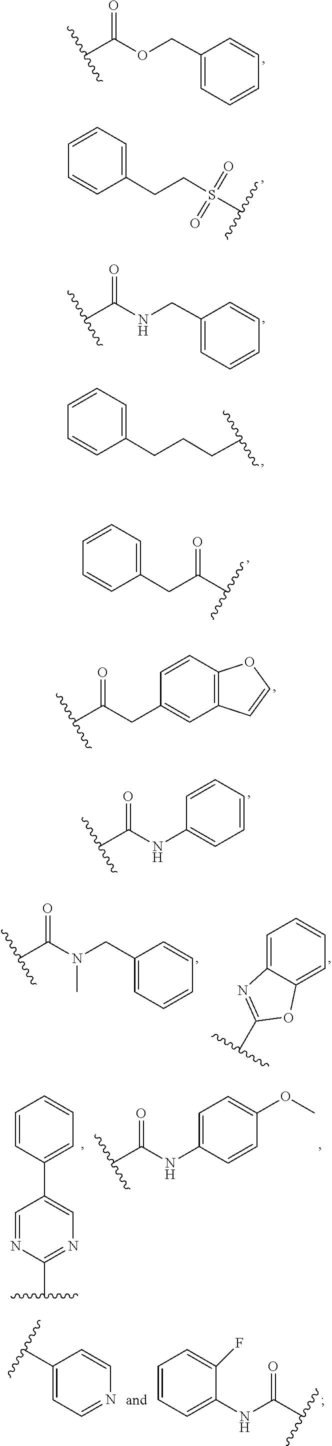

AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; X is CH₂ and Y is NH; and B is heteroaryl^{a} and selected from isoquinolinyl and azaindole (specifically 7-azaindole), optionally substituted as for heteroaryl^{a}.

More specifically, -V-Z- is selected from —CH₂—, —O—CH₂—, —CH₂—O— and —CH₂—CH₂—O—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; X is CH₂ and Y is NH; and B is heteroaryl^{a} and selected from isoquinolinyl and azaindole (specifically 7-azaindole), optionally substituted as for heteroaryl^{a}.

More specifically, AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 6- to 12-membered bicyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein the bicyclic ring system is fused, bridged or spiro; X is CH₂ and Y is NH; and B is heteroaryl^{a} and selected from isoquinolinyl and azaindole (specifically 7-azaindole), optionally substituted as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A;
and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH or N; and B is heteroaryl^{a} and selected from isoquinolinyl and azaindole (specifically 7-azaindole), optionally substituted as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH or N; and B is heteroaryl^{a} and selected from isoquinolinyl and azaindole (specifically 7-azaindole), optionally substituted as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH or N; and B is heteroaryl^{a} and selected from isoquinolinyl and azaindole (specifically 7-azaindole), optionally substituted as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH; X is CH₂ and Y is NH; and B is heteroaryl^{a} and selected from isoquinolinyl and azaindole (specifically 7-azaindole), optionally substituted as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH; X is CH₂ and Y is NH; and B is heteroaryl^{a} and selected from isoquinolinyl and azaindole (specifically 7-azaindole), optionally substituted as for heteroaryl^{a}.

Alternatively, -V-Z- is selected from from —CH₂—, —O—CH₂—, —CH₂—O—, —CH₂—CH₂—O—, —CH₂—N(R18)- and —N(R18)-CH₂—, wherein R18 is selected from:

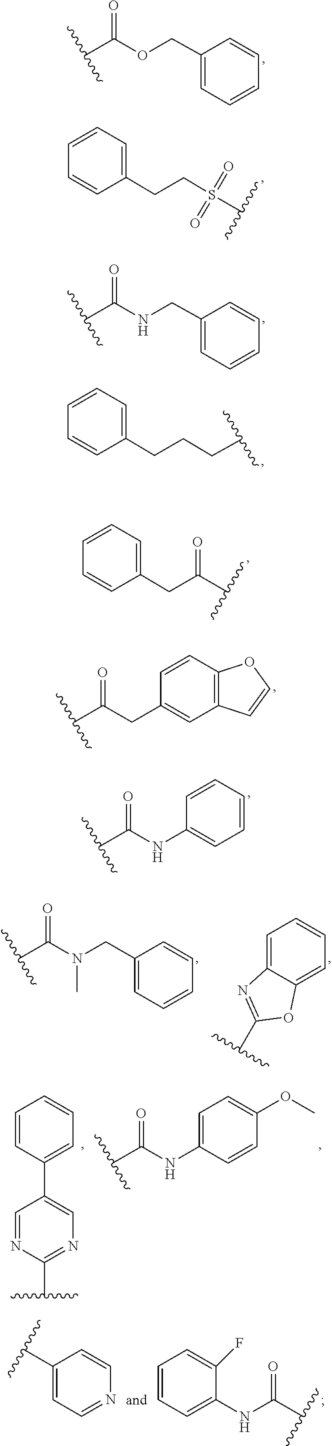

AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH; X is CH₂ and Y is NH; and B is heteroaryl^{a} and selected from isoquinolinyl and azaindole (specifically 7-azaindole), optionally substituted as for heteroaryl^{a}.

Alternatively, -V-Z- is selected from —CH₂—, —O—CH₂—, —CH₂—O— and —CH₂—CH₂—O—;
AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH; X is CH₂ and Y is NH; and B is heteroaryl^{a} and selected from isoquinolinyl and azaindole (specifically 7-azaindole), optionally substituted as for heteroaryl^{a}.

Alternatively, AW- is selected from:
—(CH₂)₀₋₃-A, —(CH₂)₀₋₃—O—(CH₂)₀₋₃-A, —(CH₂)₀₋₃-A, —(CH₂)₀₋₃—NH—(CH₂)₀₋₃-A and —(CH₂)₀₋₃—NR12-(CH₂)₁₋₃—C(═O)-A; and A is a 4- to 7-membered monocyclic ring system, containing one N ring member and optionally one or two further ring members independently selected from N, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN. A can be a 4- to 7-membered monocyclic ring system, containing one N ring member and optionally one or two further ring members independently selected from N, optionally wherein the ring system is substituted, where possible, with 1, 2, 3 or 4 substituents independently selected from alkyl and cycloalkyl. A can be a 4- to 7-membered monocyclic ring system, containing one N ring member and optionally one further ring member independently selected from N, optionally wherein the ring system is substituted, where possible, with 1, 2, 3 or 4 substituents independently selected from alkyl and cycloalkyl; and X is CH₂ and Y is NH; X is CH₂ and Y is NH; and B is heteroaryl^{a} and selected from isoquinolinyl and azaindole (specifically 7-azaindole), optionally substituted as for heteroaryl^{a}.

More preferably, AW- is selected from:
—(CHR12)₀₋₆-A (e.g. —(CHR12)₀₋₃-A, specifically —(CH₂)-A) and —(CH₂)₀₋₆—O—(CH₂)₀₋₆-A (e.g. —(CH₂)₀₋₃—O—(CH₂)_{0- 3}-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH or N; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH or N; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic; X is CH₂ and Y is NH or N; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

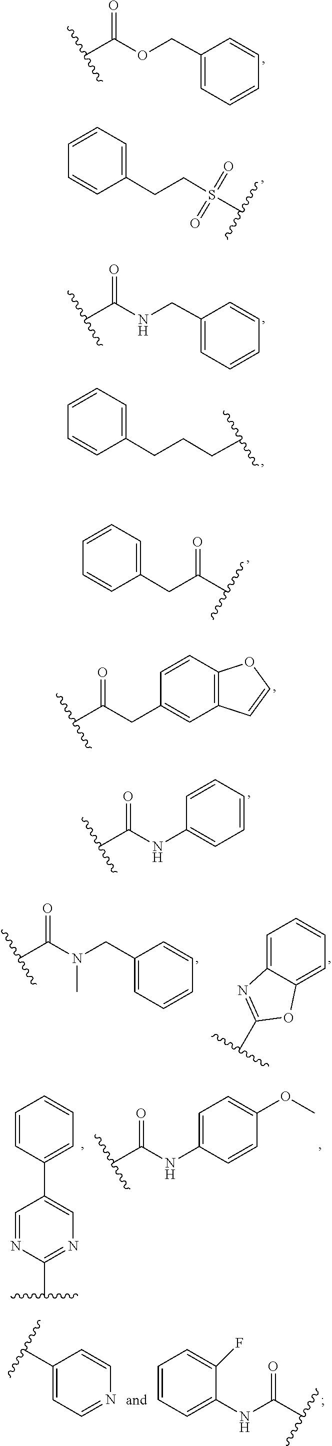

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

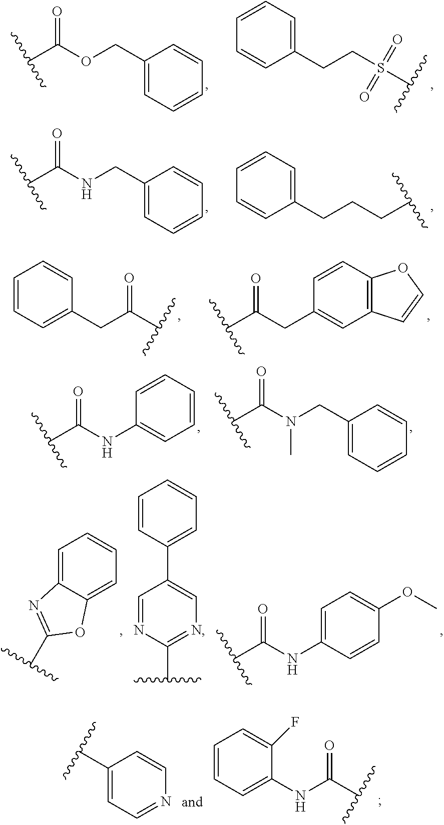

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically azaindole (particularly 7-azaindole), substituted with methyl, and optionally substituted with 1 or 2 further substituents as for heteroaryl^{a}.

More preferably, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}. Alternatively, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

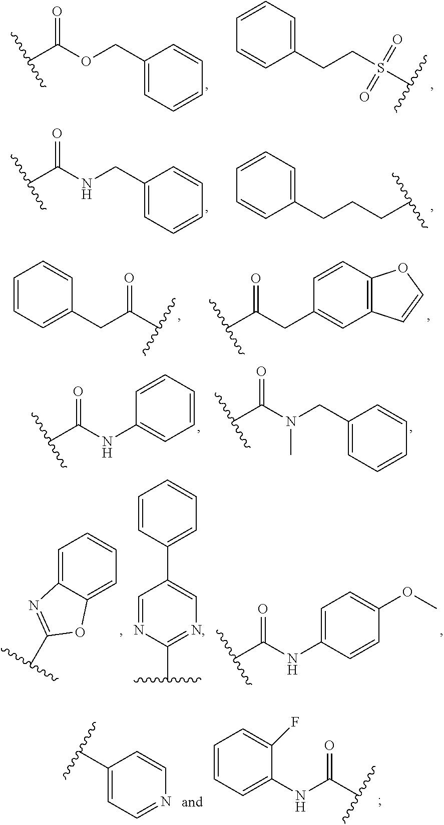

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

More preferably, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

More preferably, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

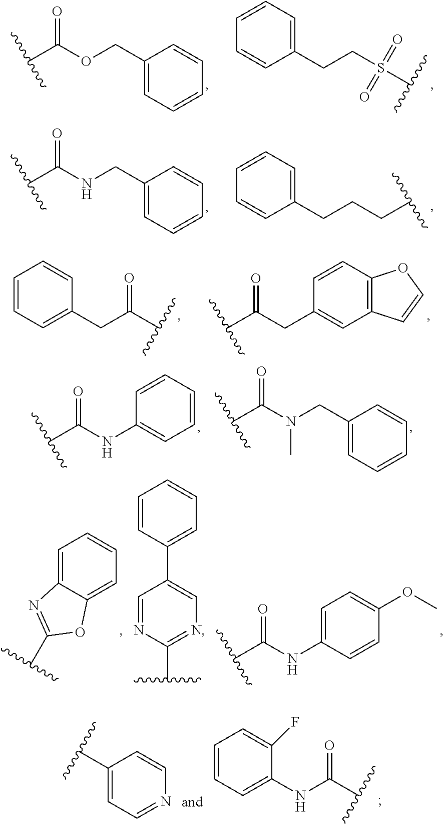

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

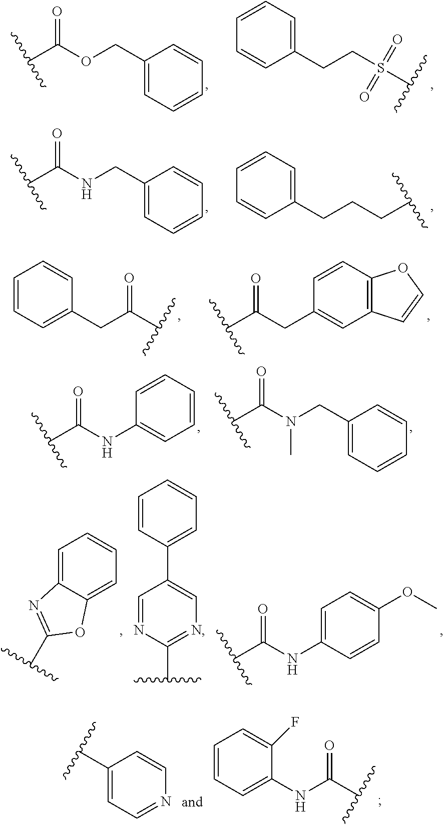

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}. Alternatively, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

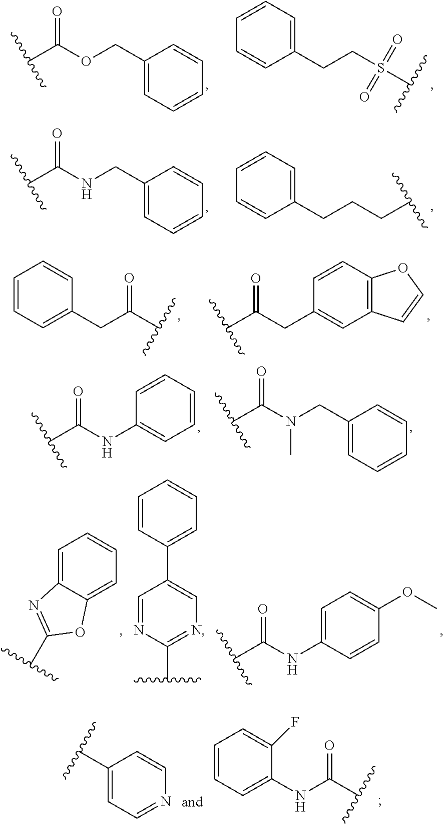

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

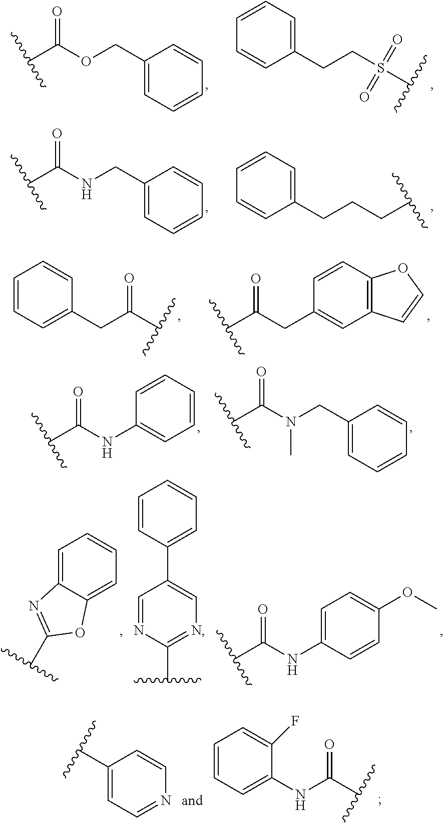

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}. Alternatively, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 9- or 10-membered bicyclic ring system (particularly 9-membered) containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; wherein one of the rings in the bicyclic ring system can be aromatic or both of the rings in the bicyclic ring system can be aromatic; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; and B is heteroaryl^{a}, specifically azaindole (particularly 7-azaindole),), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; and B is heteroaryl^{a}, specifically azaindole (particularly 7-azaindole),), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; and B is heteroaryl^{a}, specifically azaindole (particularly 7-azaindole),), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically azaindole (particularly 7-azaindole),), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically azaindole (particularly 7-azaindole),), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

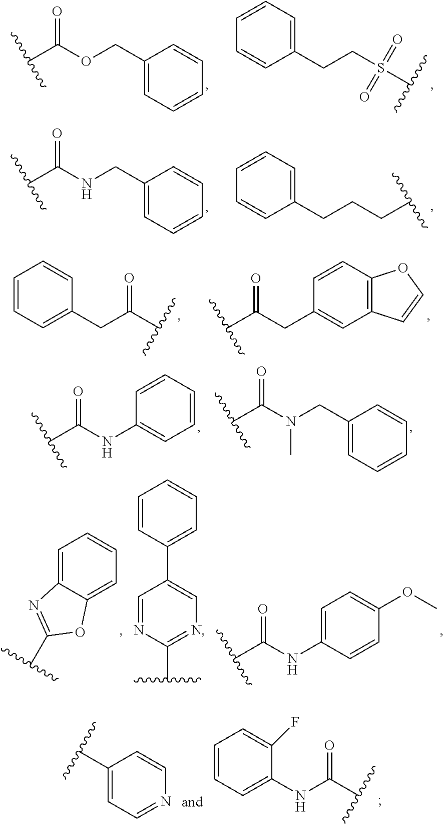

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

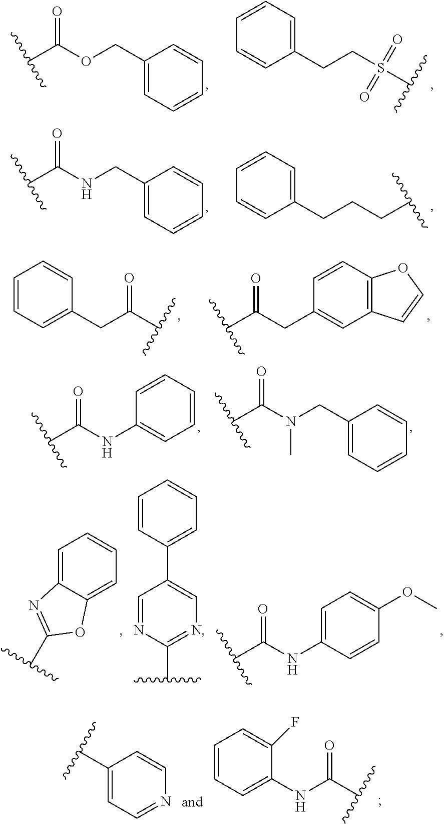

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically azaindole (particularly 7-azaindole),), substituted with methyl, and optionally substituted with 1 or 2 further substituents as for heteroaryl^{a}.

More preferably, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically azaindole (particularly 7-azaindole),), substituted with methyl, and optionally substituted with 1 or 2 further substituents as for heteroaryl^{a}.

More preferably, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}. Alternatively, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically azaindole (particularly 7-azaindole),), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH or N; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13,
C(═O)NR13R14, NR13R14, CF₃, CN; and X is CH₂ and Y is NH; X is CH₂ and Y is NH or N; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH or N; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH or N; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from —CH₂—, —O—CH₂—, —CH₂—O—, —CH₂—CH₂—O—, —CH₂—N(R18)- and —N(R18)-CH₂—, wherein R18 is selected from:

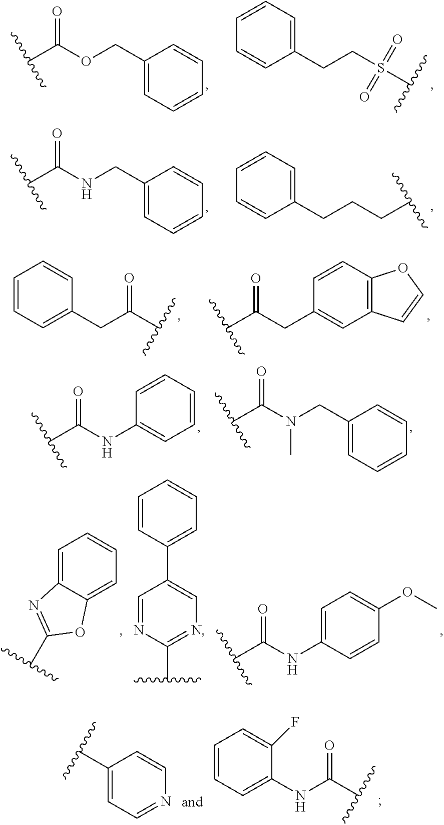

wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

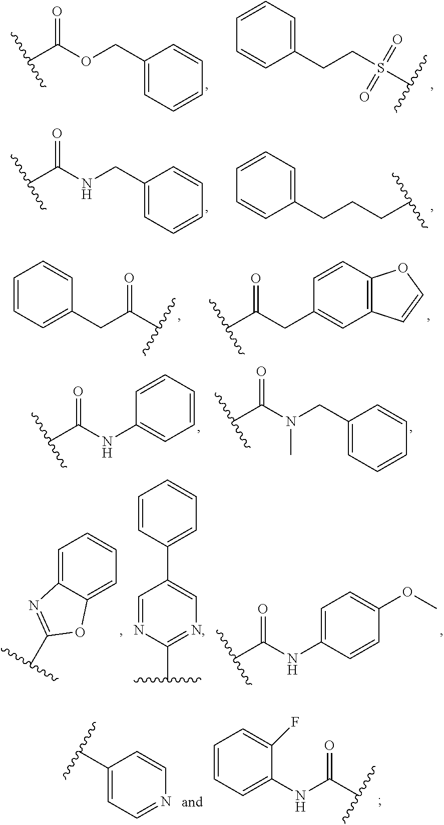

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically azaindole (particularly 7-azaindole), substituted with methyl, and optionally substituted with 1 or 2 further substituents as for heteroaryl^{a}.

More preferably, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}. Alternatively, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is NH; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

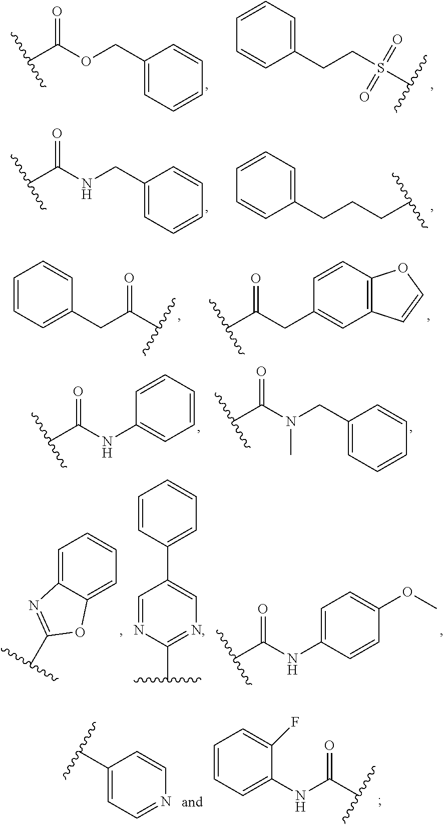

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

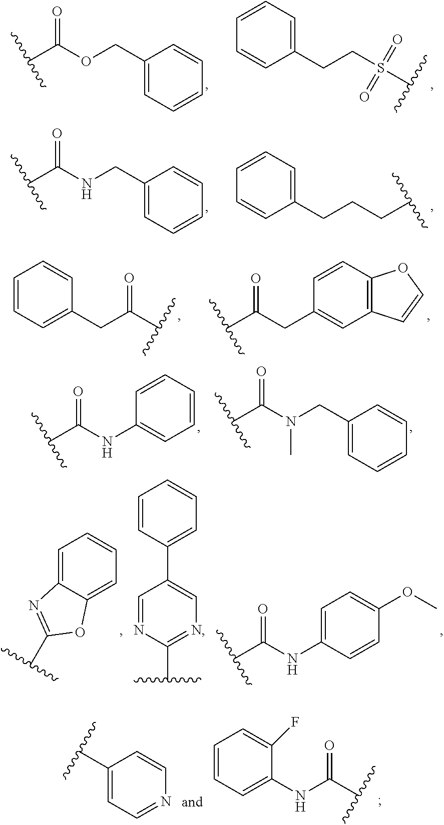

AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O— and —CH₂—CH₂—O—;
AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}. Alternatively, AW- is selected from:
—(CHR12)-A (specifically —(CH₂)-A), —(CH₂)₀₋₆—C(═O)—(CH₂)₀₋₆-A (e.g. —(CH₂)₀₋₃—C(═O)—(CH₂)₀₋₃-A, specifically —C(═O)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH or N; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂— —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13,
C(═O)NR13R14, NR13R14, CF₃, CN; and X is CH₂ and Y is NH or N; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, when not absent, -V-Z- is selected from: —CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or, when -V-Z- is absent: U is absent, CH₂ or —CH₂CH₂—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from (CH₂)₀₋₆-aryl and (CH₂)₀₋₆-heteroaryl^{a};
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O—, —CH₂—CH₂—O—, —CH₂—N(R18)- and —N(R18)-CH₂—, wherein R18 is selected from:

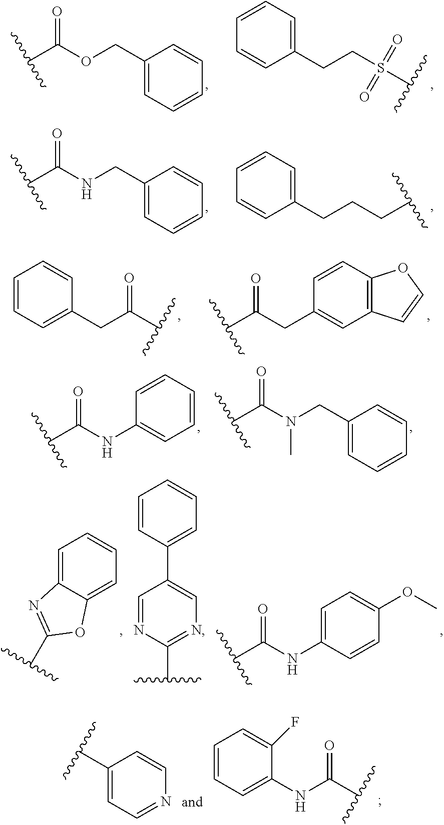

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O—, —CH₂—CH₂—O—, —CH₂—N(R18)- and —N(R18)-CH₂—, wherein R18 is selected from:

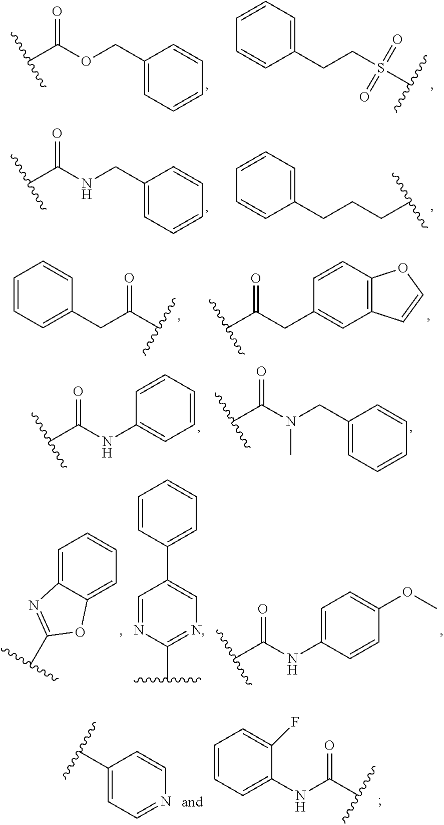

AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}.

Alternatively, -V-Z- is selected from —CH₂—, —O—CH₂—, —CH₂—O— and —CH₂—CH₂—O—;
AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically azaindole (particularly 7-azaindole), substituted with methyl, and optionally substituted with 1 or 2 further substituents as for heteroaryl^{a}.

More preferably, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically isoquinolinyl substituted with —NH₂, and optionally 1 or 2 further substituent as for heteroaryl^{a}, or B is heteroaryl^{a}, specifically azaindole (particularly 7-azaindole), substituted with halo (particularly, chloro), and optionally 1 or 2 further substituent as for heteroaryl^{a}. Alternatively, AW- is selected from:
—(CHR12)-A (specifically —(CH₂)-A) and —(CH₂)₀₋₆—O—(CH₂)₀₋₆-A (e.g. —(CH₂)₀₋₃—O—(CH₂)₀₋₃-A, specifically —(CH₂)—O-A); and A is a 6-membered monocyclic ring system containing one N ring member, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN; and X is CH₂ and Y is NH; X is CH₂ and Y is O; and B is heteroaryl^{a}, specifically azaindole (particularly 7-azaindole), substituted with methyl, and optionally substituted with 1 or 2 further substituents as for heteroaryl^{a}.

For the compounds provided in Table 1a, Table 1b, Table 2a, Table 2b, Table 3a, Table 4a, Table 5b, Table 6a, Table 6b, Table 7a, Table 7b, Table 8a, Table 8b, Table 9b, Table 10a, Table 10b, Table 11b, Table 12b and Table 13b below, where stereochemistry is indicated, the compound is intended to cover all possible stereoisomers thereof.

The present invention therefore provides the compounds below in Table 1a, and pharmaceutically acceptable salts and/or solvates thereof. The present invention therefore also provides stereoisomers of the compounds below in Table 1a, and pharmaceutically acceptable salts and/or solvates thereof.

The present invention therefore provides the compounds below in Table 2a, and pharmaceutically acceptable salts and/or solvates thereof. The present invention therefore also provides stereoisomers of the compounds below in Table 2a, and pharmaceutically acceptable salts and/or solvates thereof.

The present invention therefore provides the compounds below in Table 2b, and pharmaceutically acceptable salts and/or solvates thereof. The present invention therefore also provides stereoisomers of the compounds below in Table 2b, and pharmaceutically acceptable salts and/or solvates thereof.

The present invention therefore provides the compounds below in Table 3a, and pharmaceutically acceptable salts and/or solvates thereof. The present invention therefore also provides stereoisomers of the compounds below in Table 3a, and pharmaceutically acceptable salts and/or solvates thereof.

The present invention therefore provides the compounds below in Table 4a, and pharmaceutically acceptable salts and/or solvates thereof. The present invention therefore also provides stereoisomers of the compounds below in Table 4a, and pharmaceutically acceptable salts and/or solvates thereof.

The present invention therefore provides the compounds below in Table 5b, and pharmaceutically acceptable salts and/or solvates thereof. The present invention therefore also provides stereoisomers of the compounds below in Table 5b, and pharmaceutically acceptable salts and/or solvates thereof.

The present invention therefore provides the compounds below in Table 6a, and pharmaceutically acceptable salts and/or solvates thereof. The present invention therefore also provides stereoisomers of the compounds below in Table 6a, and pharmaceutically acceptable salts and/or solvates thereof.

The present invention therefore provides the compounds below in Table 6b, and pharmaceutically acceptable salts and/or solvates thereof. The present invention therefore also provides stereoisomers of the compounds below in Table 6b, and pharmaceutically acceptable salts and/or solvates thereof.

The present invention therefore provides the compounds below in Table 7a, and pharmaceutically acceptable salts and/or solvates thereof. The present invention therefore also provides stereoisomers of the compounds below in Table 7a, and pharmaceutically acceptable salts and/or solvates thereof.

The present invention therefore provides the compounds below in Table 7b, and pharmaceutically acceptable salts and/or solvates thereof. The present invention therefore also provides stereoisomers of the compounds below in Table 7b, and pharmaceutically acceptable salts and/or solvates thereof.

The present invention therefore provides the compounds below in Table 8a, and pharmaceutically acceptable salts and/or solvates thereof. The present invention therefore also provides stereoisomers of the compounds below in Table 8a, and pharmaceutically acceptable salts and/or solvates thereof.

The present invention therefore provides the compounds below in Table 8b, and pharmaceutically acceptable salts and/or solvates thereof. The present invention therefore also provides stereoisomers of the compounds below in Table 8b, and pharmaceutically acceptable salts and/or solvates thereof.

The present invention therefore provides the compounds below in Table 9b, and pharmaceutically acceptable salts and/or solvates thereof. The present invention therefore also provides stereoisomers of the compounds below in Table 9b, and pharmaceutically acceptable salts and/or solvates thereof.

The present invention therefore provides the compounds below in Table 10a, and pharmaceutically acceptable salts and/or solvates thereof. The present invention therefore also provides stereoisomers of the compounds below in Table 10a, and pharmaceutically acceptable salts and/or solvates thereof.

The present invention therefore provides the compounds below in Table 10b, and pharmaceutically acceptable salts and/or solvates thereof. The present invention therefore also provides stereoisomers of the compounds below in Table 10b, and pharmaceutically acceptable salts and/or solvates thereof.

The present invention therefore provides the compounds below in Table 11b, and pharmaceutically acceptable salts and/or solvates thereof. The present invention therefore also provides stereoisomers of the compounds below in Table 11b, and pharmaceutically acceptable salts and/or solvates thereof.

The present invention therefore provides the compounds below in Table 12b, and pharmaceutically acceptable salts and/or solvates thereof. The present invention therefore also provides stereoisomers of the compounds below in Table 12b, and pharmaceutically acceptable salts and/or solvates thereof.

The present invention therefore provides the compounds below in Table 13b, and pharmaceutically acceptable salts and/or solvates thereof. The present invention therefore also provides stereoisomers of the compounds below in Table 13b, and pharmaceutically acceptable salts and/or solvates thereof.

It will be understood that, when reading the compounds in Table 1a, Table 1b, Table 2a, Table 2b, Table 3a, Table 4a, Table 5b, Table 6a, Table 6b, Table 7a, Table 7b, Table 8a, Table 8b, Table 9b, Table 10a, Table 10b, Table 11b, Table 12b and Table 13b below, the substituents are to be read from left to right.

For example, example compound 1003 in Table 1a has a Q₁ group:

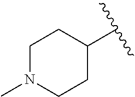 [SMILES: *C1CCN(C)CC1]

and a Q₂ group “OCH₂”. Therefore, the Q₁ group is attached to the “O” of the “OCH₂” of the Q₂ group, as follows:

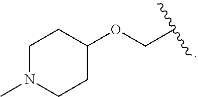 [SMILES: *COC1CCN(C)CC1]

TABLE 1a

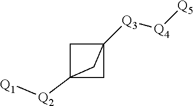

| Example | | | | | |
|---|---|---|---|---|---|
| No | Q₁ | Q₂ | Q₃ | Q₄ | Q₅ |
| 1001 | 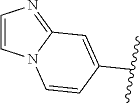 [SMILES: *c1ccn2ccnc2c1] | OCH₂ | CH₂ | NH | 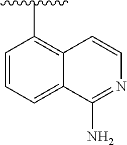 [SMILES: *c1cccc2c(N)nccc12] |
| 1002 | 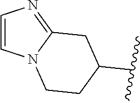 [SMILES: *C1CCn2ccnc2C1] | OCH₂ | CH₂ | NH | 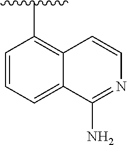 [SMILES: *c1cccc2c(N)nccc12] |
| 1002.1 | 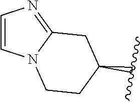 [SMILES: *[C@H]1CCn2ccnc2C1] | OCH₂ | CH₂ | NH | 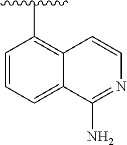 [SMILES: *c1cccc2c(N)nccc12] |
| 1002.2 | 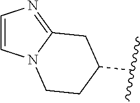 [SMILES: *[C@@H]1CCn2ccnc2C1] | OCH₂ | CH₂ | NH | 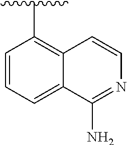 [SMILES: *c1cccc2c(N)nccc12] |
| 1003 | 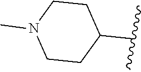 [SMILES: *C1CCN(C)CC1] | OCH₂ | CH₂ | NH | 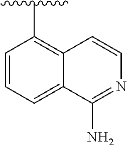 [SMILES: *c1cccc2c(N)nccc12] |
| 1004 | 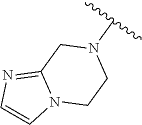 [SMILES: *N1CCn2ccnc2C1] | CH₂ | CH₂ | NH | 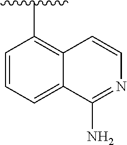 [SMILES: *c1cccc2c(N)nccc12] |
| 1005 | 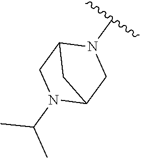 [SMILES: *N1CC2CC1CN2C(C)C] | CH₂ | CH₂ | NH | 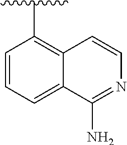 [SMILES: *c1cccc2c(N)nccc12] |
| 1005.1 | 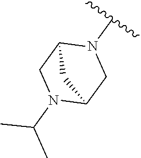 [SMILES: *N1C[C@@H]2C[C@H]1CN2C(C)C] | CH₂ | CH₂ | NH | 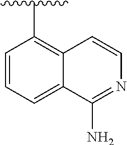 [SMILES: *c1cccc2c(N)nccc12] |
| 1005.2 | 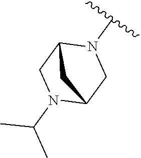 [SMILES: *N1C[C@H]2C[C@@H]1CN2C(C)C] | CH₂ | CH₂ | NH | 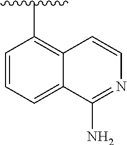 [SMILES: *c1cccc2c(N)nccc12] |
| 1006 | 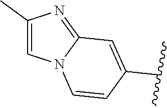 [SMILES: *c1ccn2cc(C)nc2c1] | OCH₂ | CH₂ | NH | 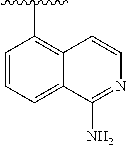 [SMILES: *c1cccc2c(N)nccc12] |
| 1007 | 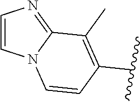 [SMILES: *c1ccn2ccnc2c1C] | OCH₂ | CH₂ | NH | 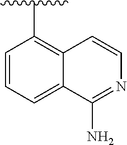 [SMILES: *c1cccc2c(N)nccc12] |
| 1008 | 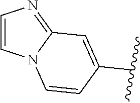 [SMILES: *c1ccn2ccnc2c1] | OCH₂ | CH₂ | NH | 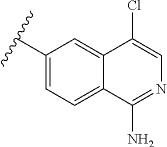 [SMILES: *c1ccc2c(N)ncc(Cl)c2c1] |
| 1009 | 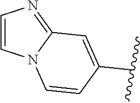 [SMILES: *c1ccn2ccnc2c1] | OCH₂ | CH₂ | NH | 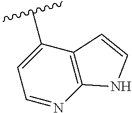 [SMILES: *c1ccnc2[nH]ccc12] |
| 1010 | 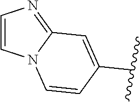 [SMILES: *c1ccn2ccnc2c1] | OCH₂ | CH₂ | NH | 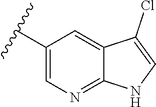 [SMILES: *c1cnc2[nH]cc(Cl)c2c1] |
| 1011 | 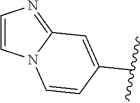 [SMILES: *c1ccn2ccnc2c1] | OCH₂ | CH₂ | NH | 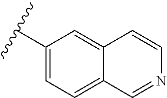 [SMILES: *c1ccc2cnccc2c1] |
| 1012 | 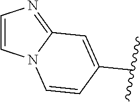 [SMILES: *c1ccn2ccnc2c1] | OCH₂ | CH₂ | NH | 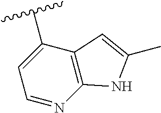 [SMILES: *c1ccnc2[nH]c(C)cc12] |
| 1013 | 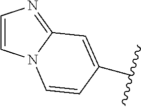 [SMILES: *c1ccn2ccnc2c1] | OCH₂ | CH₂ | O | 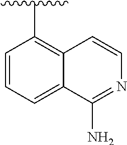 [SMILES: *c1cccc2c(N)nccc12] |
| 1014 | 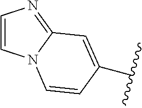 [SMILES: *c1ccn2ccnc2c1] | OCH₂ | CH₂ | CH₂ | 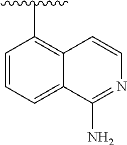 [SMILES: *c1cccc2c(N)nccc12] |
| 1015 | 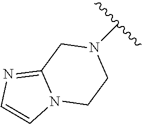 [SMILES: *N1CCn2ccnc2C1] | C═O | CH₂ | O | 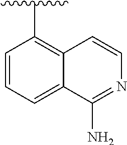 [SMILES: *c1cccc2c(N)nccc12] |
| 1016 | 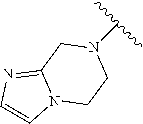 [SMILES: *N1CCn2ccnc2C1] | C═O | CH₂ | NH | 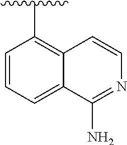 [SMILES: *c1cccc2c(N)nccc12] |
| 1017 | 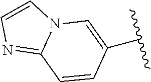 [SMILES: *c1ccc2nccn2c1] | OCH₂ | CH₂ | NH | 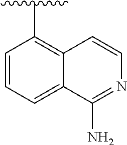 [SMILES: *c1cccc2c(N)nccc12] |
| 1018 | 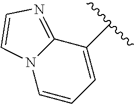 [SMILES: *c1cccn2ccnc12] | OCH₂ | CH₂ | NH | 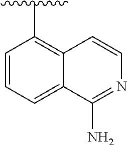 [SMILES: *c1cccc2c(N)nccc12] |
| 1019 | 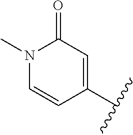 [SMILES: *c1ccn(C)c(=O)c1] | OCH₂ | CH₂ | NH | 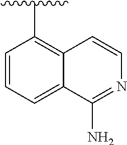 [SMILES: *c1cccc2c(N)nccc12] |
| 1101 | 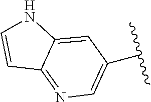 [SMILES: *c1cnc2cc[nH]c2c1] | OCH₂ | CH₂ | NH | 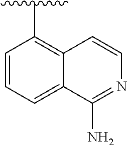 [SMILES: *c1cccc2c(N)nccc12] |
| 1104 | 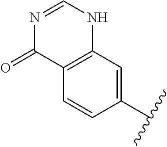 [SMILES: *c1ccc2c(=O)nc[nH]c2c1] | OCH₂ | CH₂ | NH | 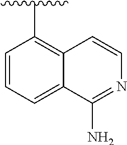 [SMILES: *c1cccc2c(N)nccc12] |
| 1105 | 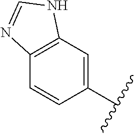 [SMILES: *c1ccc2nc[nH]c2c1] | OCH₂ | CH₂ | NH | 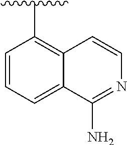 [SMILES: *c1cccc2c(N)nccc12] |
| 1106 | 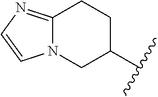 [SMILES: *C1CCc2nccn2C1] | OCH₂ | CH₂ | NH | 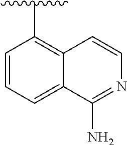 [SMILES: *c1cccc2c(N)nccc12] |
| 1108 | 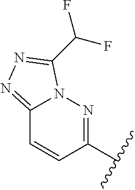 [SMILES: *c1ccc2nnc(C(F)F)n2n1] | OCH₂ | CH₂ | NH | 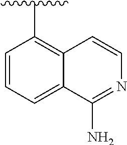 [SMILES: *c1cccc2c(N)nccc12] |
| 1109 | 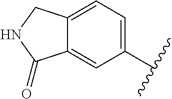 [SMILES: *c1ccc2c(c1)C(=O)NC2] | OCH₂ | CH₂ | NH | 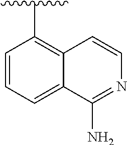 [SMILES: *c1cccc2c(N)nccc12] |
| 1110 | 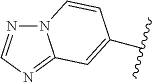 [SMILES: *c1ccn2ncnc2c1] | OCH₂ | CH₂ | NH | 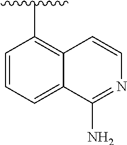 [SMILES: *c1cccc2c(N)nccc12] |
| 1112 | 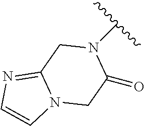 [SMILES: *N1Cc2nccn2CC1=O] | CH₂ | CH₂ | NH | 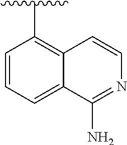 [SMILES: *c1cccc2c(N)nccc12] |
| 1113 | 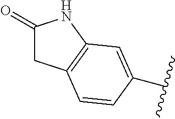 [SMILES: *c1ccc2c(c1)NC(=O)C2] | OCH₂ | CH₂ | NH | 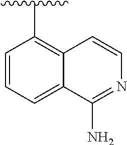 [SMILES: *c1cccc2c(N)nccc12] |
| 1114 | 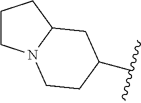 [SMILES: *C1CCN2CCCC2C1] | OCH₂ | CH₂ | NH | 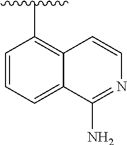 [SMILES: *c1cccc2c(N)nccc12] |
| 1115 | 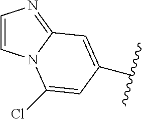 [SMILES: *c1cc(Cl)n2ccnc2c1] | OCH₂ | CH₂ | NH | 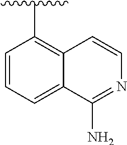 [SMILES: *c1cccc2c(N)nccc12] |
| 1116 | 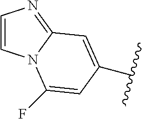 [SMILES: *c1cc(F)n2ccnc2c1] | OCH₂ | CH₂ | NH | 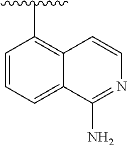 [SMILES: *c1cccc2c(N)nccc12] |
| 1117 | 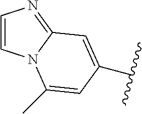 [SMILES: *c1cc(C)n2ccnc2c1] | OCH₂ | CH₂ | NH | 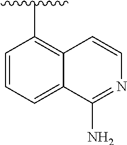 [SMILES: *c1cccc2c(N)nccc12] |
| 1118 | 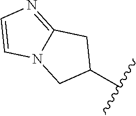 [SMILES: *C1Cc2nccn2C1] | OCH₂ | CH₂ | NH | 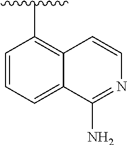 [SMILES: *c1cccc2c(N)nccc12] |
| 1119 | 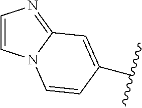 [SMILES: *c1ccn2ccnc2c1] | CH₂O | CH₂ | NH | 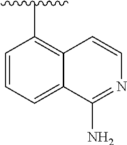 [SMILES: *c1cccc2c(N)nccc12] |
| 1120 | 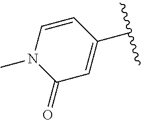 [SMILES: *c1ccn(C)c(=O)c1] | OCH₂ | absent | NH | 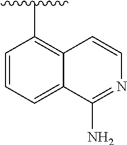 [SMILES: *c1cccc2c(N)nccc12] |
| 1121 | 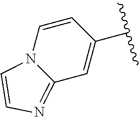 [SMILES: *c1ccn2ccnc2c1] | OCH₂ | absent | NH | 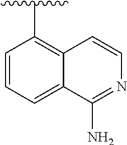 [SMILES: *c1cccc2c(N)nccc12] |
| 1122 | 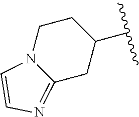 [SMILES: *C1CCn2ccnc2C1] | OCH₂ | absent | NH | 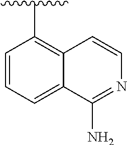 [SMILES: *c1cccc2c(N)nccc12] |
| 1123 | 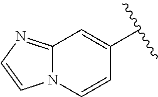 [SMILES: *c1ccn2ccnc2c1] | CH₂O | absent | NH | 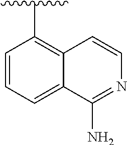 [SMILES: *c1cccc2c(N)nccc12] |
| 1124 | 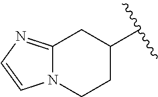 [SMILES: *C1CCn2ccnc2C1] | CH₂O | absent | NH | 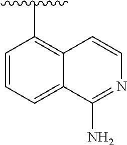 [SMILES: *c1cccc2c(N)nccc12] |

TABLE 1b

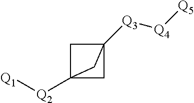

| Example | | | | | |
|---|---|---|---|---|---|
| No | Q₁ | Q₂ | Q₃ | Q₄ | Q₅ |
| 1125 | 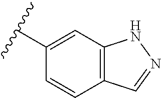 [SMILES: *c1ccc2cn[nH]c2c1] | OCH₂ | CH₂ | NH | 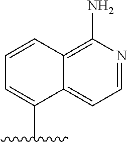 [SMILES: *c1cccc2c(N)nccc12] |
| 1126 | 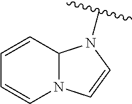 [SMILES: *N1C=CN2C=CC=CC12] | CH₂ | CH₂ | NH | 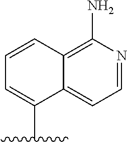 [SMILES: *c1cccc2c(N)nccc12] |
| 1127 | 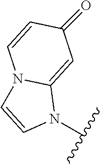 [SMILES: *n1ccn2ccc(=O)cc12] | CH₂ | CH₂ | NH | 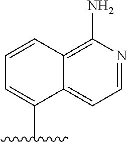 [SMILES: *c1cccc2c(N)nccc12] |
| 1129 | 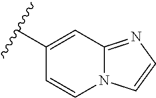 [SMILES: *c1ccn2ccnc2c1] | OCH₂ | CH₂ | NH | 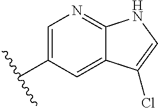 [SMILES: *c1cnc2[nH]cc(Cl)c2c1] |
| 1130 | 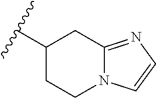 [SMILES: *C1CCn2ccnc2C1] | OCH₂ | CH₂ | NH | 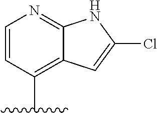 [SMILES: *c1ccnc2[nH]c(Cl)cc12] |
| 1131 | 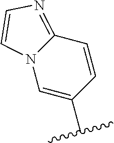 [SMILES: *c1ccc2nccn2c1] | OCH₂ | Absent | NH | 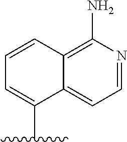 [SMILES: *c1cccc2c(N)nccc12] |
| 1132 | 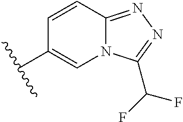 [SMILES: *c1ccc2nnc(C(F)F)n2c1] | CONH | CH₂ | NH | 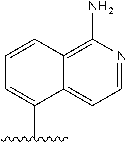 [SMILES: *c1cccc2c(N)nccc12] |
| 1133 | 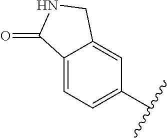 [SMILES: *c1ccc2c(c1)CNC2=O] | OCH₂ | CH₂ | NH | 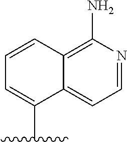 [SMILES: *c1cccc2c(N)nccc12] |
| 1134 | 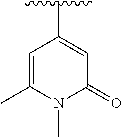 [SMILES: *c1cc(C)n(C)c(=O)c1] | OCH₂ | CH₂ | NH | 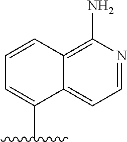 [SMILES: *c1cccc2c(N)nccc12] |
| 1135 | 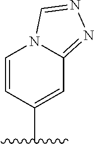 [SMILES: *c1ccn2cnnc2c1] | OCH₂ | CH₂ | NH | 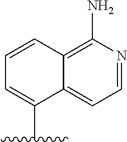 [SMILES: *c1cccc2c(N)nccc12] |

TABLE 2a

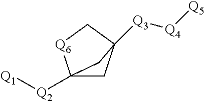

| Example No | Q₁ | Q₂ | Q₃ | Q₄ | Q₅ | Q₆ |
|---|---|---|---|---|---|---|
| 2020 | 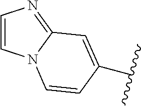 [SMILES: *c1ccn2ccnc2c1] | OCH₂ | CH₂ | NH | 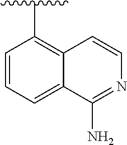 [SMILES: *c1cccc2c(N)nccc12] | O |
| 2021 | 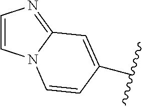 [SMILES: *c1ccn2ccnc2c1] | OCH₂ | CH₂ | NH | 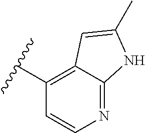 [SMILES: *c1ccnc2[nH]c(C)cc12] | O |
| 2022 | 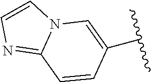 [SMILES: *c1ccc2nccn2c1] | OCH₂ | CH₂ | NH | 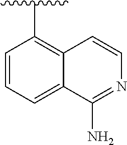 [SMILES: *c1cccc2c(N)nccc12] | O |
| 2201 | 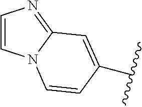 [SMILES: *c1ccn2ccnc2c1] | OCH₂ | CH₂ | O | 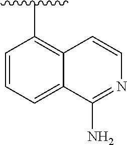 [SMILES: *c1cccc2c(N)nccc12] | NH |
| 2202 | 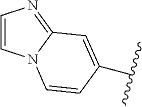 [SMILES: *c1ccn2ccnc2c1] | OCH₂ | CH₂ | O | 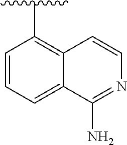 [SMILES: *c1cccc2c(N)nccc12] | NCOCH₃ |
| 2203 | 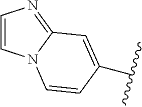 [SMILES: *c1ccn2ccnc2c1] | OCH₂ | CH₂ | O | 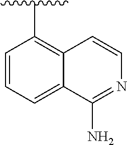 [SMILES: *c1cccc2c(N)nccc12] | CH₂ |

TABLE 2b

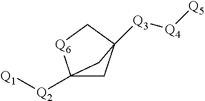

| Example No | Q₁ | Q₂ | Q₃ | Q₄ | Q₅ | Q₆ |
|---|---|---|---|---|---|---|
| 2204 | 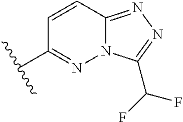 [SMILES: *c1ccc2nnc(C(F)F)n2n1] | OCH₂ | CH₂ | NH | 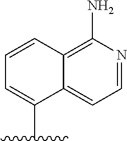 [SMILES: *c1cccc2c(N)nccc12] | O |
| 2205 | 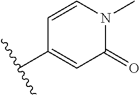 [SMILES: *c1ccn(C)c(=O)c1] | OCH₂ | CH₂ | NH | 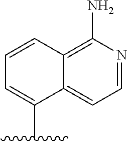 [SMILES: *c1cccc2c(N)nccc12] | O |
| 2206 | 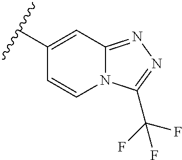 [SMILES: *c1ccn2c(C(F)(F)F)nnc2c1] | OCH₂ | CH₂ | NH | 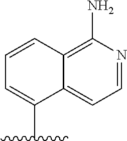 [SMILES: *c1cccc2c(N)nccc12] | O |
| 2207 | 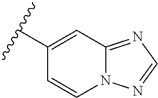 [SMILES: *c1ccn2ncnc2c1] | OCH₂ | CH₂ | NH | 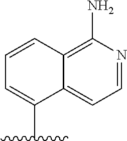 [SMILES: *c1cccc2c(N)nccc12] | O |
| 2208 | 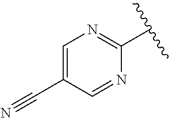 [SMILES: *c1ncc(C#N)cn1] | OCH₂ | CH₂ | NH | 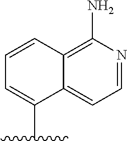 [SMILES: *c1cccc2c(N)nccc12] | O |
| 2210 | 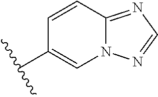 [SMILES: *c1ccc2ncnn2c1] | OCH₂ | CH₂ | NH | 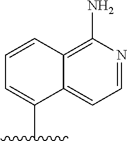 [SMILES: *c1cccc2c(N)nccc12] | O |
| 2211 | 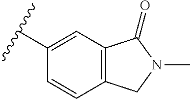 [SMILES: *c1ccc2c(c1)C(=O)N(C)C2] | OCH₂ | CH₂ | NH | 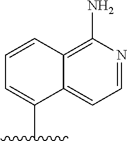 [SMILES: *c1cccc2c(N)nccc12] | O |
| 2212 | 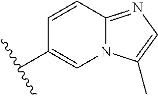 [SMILES: *c1ccc2ncc(C)n2c1] | OCH₂ | CH₂ | NH | 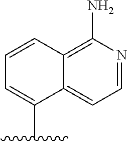 [SMILES: *c1cccc2c(N)nccc12] | O |

TABLE 3a

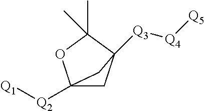

| Ex- | | | | | |
|---|---|---|---|---|---|
| ample | | | | | |
| No | Q₁ | Q₂ | Q₃ | Q₄ | Q₅ |
| 3023 | 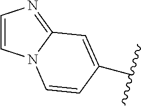 [SMILES: *c1ccn2ccnc2c1] | OCH₂ | CH₂ | NH | 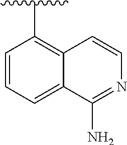 [SMILES: *c1cccc2c(N)nccc12] |
| 3024 | 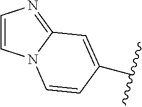 [SMILES: *c1ccn2ccnc2c1] | OCH₂ | CH₂ | O | 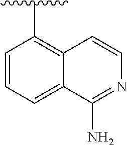 [SMILES: *c1cccc2c(N)nccc12] |

TABLE 4a

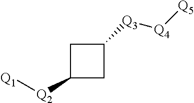

| Ex- | | | | | |
|---|---|---|---|---|---|
| ample | | | | | |
| No | Q₁ | Q₂ | Q₃ | Q₄ | Q₅ |
| 4401 | 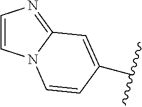 [SMILES: *c1ccn2ccnc2c1] | OCH₂ | CH₂ | NH | 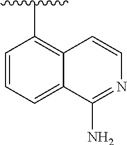 [SMILES: *c1cccc2c(N)nccc12] |
| 4402 | 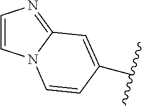 [SMILES: *c1ccn2ccnc2c1] | CH₂O | CH₂ | NH | 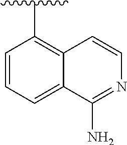 [SMILES: *c1cccc2c(N)nccc12] |
| 4403 | 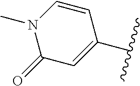 [SMILES: *c1ccn(C)c(=O)c1] | CH₂O | CH₂ | NH | 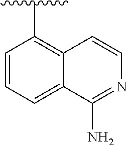 [SMILES: *c1cccc2c(N)nccc12] |

TABLE 5b

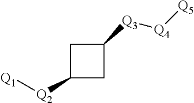

| Example No | Q₁ | Q₂ | Q₃ | Q₄ | Q₅ | |
|---|---|---|---|---|---|---|
| 5003 | 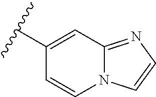 [SMILES: *c1ccn2ccnc2c1] | OCH₂ | CH₂ | NH | 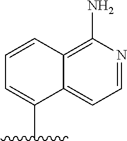 [SMILES: *c1cccc2c(N)nccc12] | 242.7 |

TABLE 6a

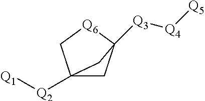

| Example | | | | | | |
|---|---|---|---|---|---|---|
| No | Q₁ | Q₂ | Q₃ | Q₄ | Q₅ | Q₆ |
| 6601 | 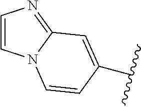 [SMILES: *c1ccn2ccnc2c1] | OCH₂ | CH₂ | NH | 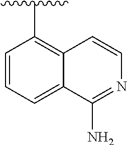 [SMILES: *c1cccc2c(N)nccc12] | O |
| 6602 | 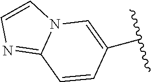 [SMILES: *c1ccc2nccn2c1] | OCH₂ | CH₂ | NH | 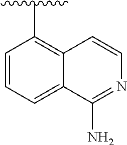 [SMILES: *c1cccc2c(N)nccc12] | O |
| 6603 | 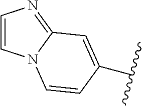 [SMILES: *c1ccn2ccnc2c1] | OCH₂ | CH₂ | O | 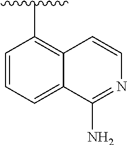 [SMILES: *c1cccc2c(N)nccc12] | NH |
| 6604 | 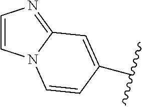 [SMILES: *c1ccn2ccnc2c1] | OCH₂ | CH₂ | O | 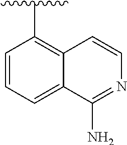 [SMILES: *c1cccc2c(N)nccc12] | NCOCH₃ |
| 6605 | 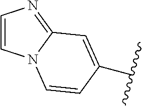 [SMILES: *c1ccn2ccnc2c1] | OCH₂ | CH₂ | NH | 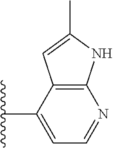 [SMILES: *c1ccnc2[nH]c(C)cc12] | O |
| 6606 | 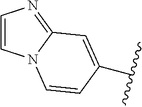 [SMILES: *c1ccn2ccnc2c1] | OCH₂ | CH₂ | NH | 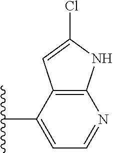 [SMILES: *c1ccnc2[nH]c(Cl)cc12] | O |
| 6607 | 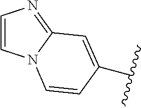 [SMILES: *c1ccn2ccnc2c1] | OCH₂ | CH₂ | NH | 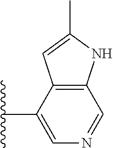 [SMILES: *c1cncc2[nH]c(C)cc12] | O |

TABLE 6b

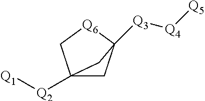

| Example | | | | | | |
|---|---|---|---|---|---|---|
| No | Q₁ | Q₂ | Q₃ | Q₄ | Q₅ | Q₆ |
| 6608 | 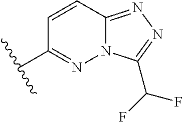 [SMILES: *c1ccc2nnc(C(F)F)n2n1] | OCH₂ | CH₂ | NH | 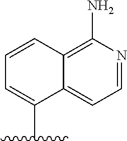 [SMILES: *c1cccc2c(N)nccc12] | O |
| 6609 | 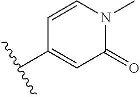 [SMILES: *c1ccn(C)c(=O)c1] | OCH₂ | CH₂ | NH | 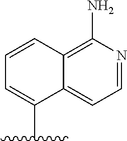 [SMILES: *c1cccc2c(N)nccc12] | O |
| 6610 | 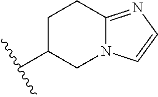 [SMILES: *C1CCc2nccn2C1] | OCH₂ | CH₂ | NH | 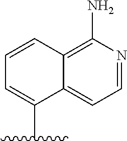 [SMILES: *c1cccc2c(N)nccc12] | O |
| 6611 | 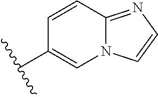 [SMILES: *c1ccc2nccn2c1] | OCH₂ | CH₂ | NH | 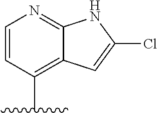 [SMILES: *c1ccnc2[nH]c(Cl)cc12] | O |
| 6612 | 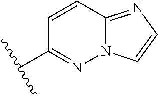 [SMILES: *c1ccc2nccn2n1] | OCH₂ | CH₂ | NH | 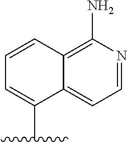 [SMILES: *c1cccc2c(N)nccc12] | O |
| 6613 | 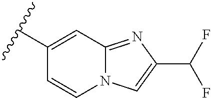 [SMILES: *c1ccn2cc(C(F)F)nc2c1] | OCH₂ | CH₂ | NH | 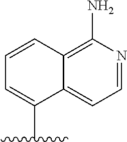 [SMILES: *c1cccc2c(N)nccc12] | O |
| 6614 | 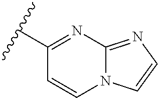 [SMILES: *c1ccn2ccnc2n1] | OCH₂ | CH₂ | NH | 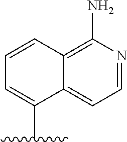 [SMILES: *c1cccc2c(N)nccc12] | O |
| 6615 | 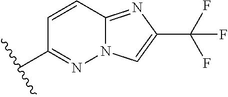 [SMILES: *c1ccc2nc(C(F)(F)F)cn2n1] | OCH₂ | CH₂ | NH | 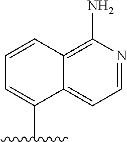 [SMILES: *c1cccc2c(N)nccc12] | O |
| 6616 | 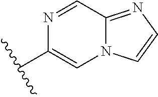 [SMILES: *c1cn2ccnc2cn1] | OCH₂ | CH₂ | NH | 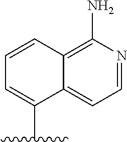 [SMILES: *c1cccc2c(N)nccc12] | O |
| 6617 | 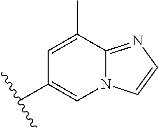 [SMILES: *c1cc(C)c2nccn2c1] | OCH₂ | CH₂ | NH | 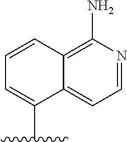 [SMILES: *c1cccc2c(N)nccc12] | O |
| 6618 | 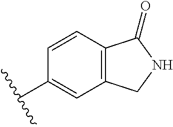 [SMILES: *c1ccc2c(c1)CNC2=O] | OCH₂ | CH₂ | NH | 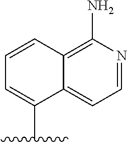 [SMILES: *c1cccc2c(N)nccc12] | O |
| 6619 | 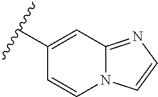 [SMILES: *c1ccn2ccnc2c1] | NHCH₂ | CH₂ | O | 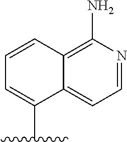 [SMILES: *c1cccc2c(N)nccc12] | O |
| 6620 | 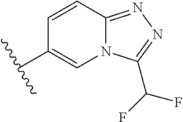 [SMILES: *c1ccc2nnc(C(F)F)n2c1] | OCH₂ | CH₂ | NH | 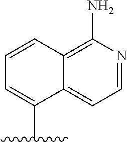 [SMILES: *c1cccc2c(N)nccc12] | O |
| 6621 | 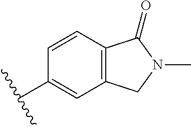 [SMILES: *c1ccc2c(c1)CN(C)C2=O] | OCH₂ | CH₂ | NH | 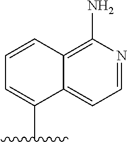 [SMILES: *c1cccc2c(N)nccc12] | O |
| 6622 | 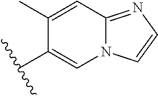 [SMILES: *c1cn2ccnc2cc1C] | OCH₂ | CH₂ | NH | 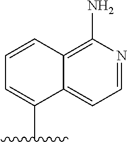 [SMILES: *c1cccc2c(N)nccc12] | O |
| 6623 | 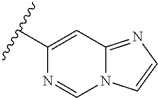 [SMILES: *c1cc2nccn2cn1] | OCH₂ | CH₂ | NH | 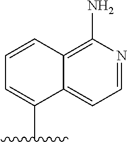 [SMILES: *c1cccc2c(N)nccc12] | O |
| 6624 | 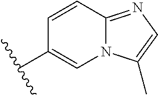 [SMILES: *c1ccc2ncc(C)n2c1] | OCH₂ | CH₂ | NH | 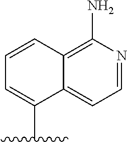 [SMILES: *c1cccc2c(N)nccc12] | O |
| 6625 | 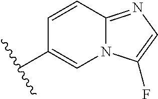 [SMILES: *c1ccc2ncc(F)n2c1] | OCH₂ | CH₂ | NH | 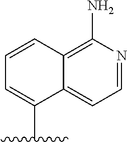 [SMILES: *c1cccc2c(N)nccc12] | O |
| 6626 | 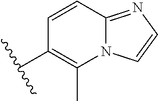 [SMILES: *c1ccc2nccn2c1C] | OCH₂ | CH₂ | NH | 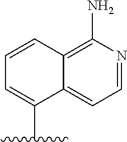 [SMILES: *c1cccc2c(N)nccc12] | O |
| 6627 | 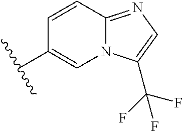 [SMILES: *c1ccc2ncc(C(F)(F)F)n2c1] | OCH₂ | CH₂ | NH | 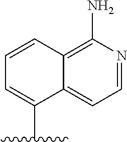 [SMILES: *c1cccc2c(N)nccc12] | O |
| 6628 | 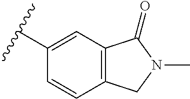 [SMILES: *c1ccc2c(c1)C(=O)N(C)C2] | OCH₂ | CH₂ | NH | 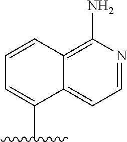 [SMILES: *c1cccc2c(N)nccc12] | O |
| 6629 | 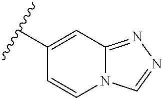 [SMILES: *c1ccn2cnnc2c1] | OCH₂ | CH₂ | NH | 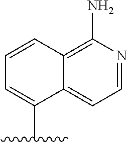 [SMILES: *c1cccc2c(N)nccc12] | O |
| 6630 | 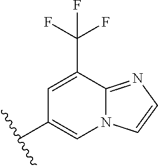 [SMILES: *c1cc(C(F)(F)F)c2nccn2c1] | OCH₂ | CH₂ | NH | 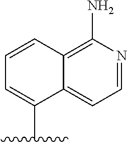 [SMILES: *c1cccc2c(N)nccc12] | O |
| 6631 | 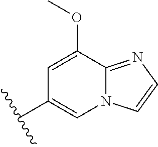 [SMILES: *c1cc(OC)c2nccn2c1] | OCH₂ | CH₂ | NH | 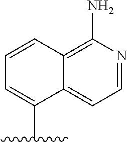 [SMILES: *c1cccc2c(N)nccc12] | O |
| 6633 | 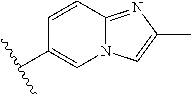 [SMILES: *c1ccc2nc(C)cn2c1] | OCH₂ | CH₂ | NH | 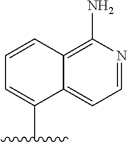 [SMILES: *c1cccc2c(N)nccc12] | O |
| 6634 | 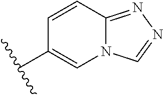 [SMILES: *c1ccc2nncn2c1] | OCH₂ | CH₂ | NH | 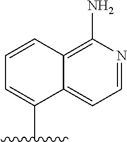 [SMILES: *c1cccc2c(N)nccc12] | O |
| 6635 | 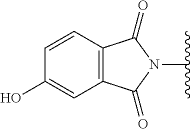 [SMILES: *N1C(=O)c2ccc(O)cc2C1=O] | CH₂ | CH₂ | NH | 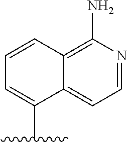 [SMILES: *c1cccc2c(N)nccc12] | O |
| 6636 | 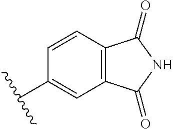 [SMILES: *c1ccc2c(c1)C(=O)NC2=O] | OCH₂ | CH₂ | NH | 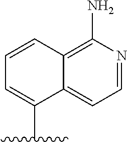 [SMILES: *c1cccc2c(N)nccc12] | O |
| 6637 | 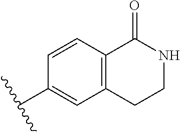 [SMILES: *c1ccc2c(c1)CCNC2=O] | OCH₂ | CH₂ | NH | 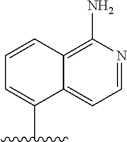 [SMILES: *c1cccc2c(N)nccc12] | O |
| 6638 | 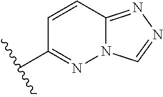 [SMILES: *c1ccc2nncn2n1] | OCH₂ | CH₂ | NH | 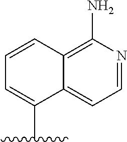 [SMILES: *c1cccc2c(N)nccc12] | O |
| 6639 | 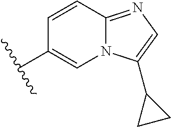 [SMILES: *c1ccc2ncc(C3CC3)n2c1] | OCH₂ | CH₂ | NH | 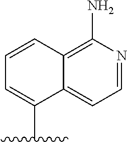 [SMILES: *c1cccc2c(N)nccc12] | O |
| 6640 | 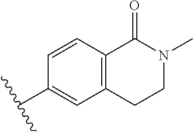 [SMILES: *c1ccc2c(c1)CCN(C)C2=O] | OCH₂ | CH₂ | NH | 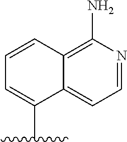 [SMILES: *c1cccc2c(N)nccc12] | O |
| 6641 | 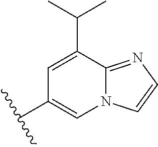 [SMILES: *c1cc(C(C)C)c2nccn2c1] | OCH₂ | CH₂ | NH | 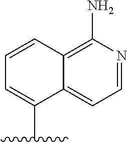 [SMILES: *c1cccc2c(N)nccc12] | O |
| 6642 | 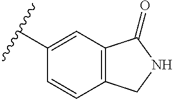 [SMILES: *c1ccc2c(c1)C(=O)NC2] | OCH₂ | CH₂ | NH | 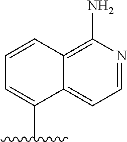 [SMILES: *c1cccc2c(N)nccc12] | O |
| 6643 | 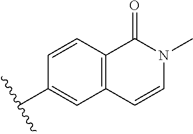 [SMILES: *c1ccc2c(=O)n(C)ccc2c1] | OCH₂ | CH₂ | NH | 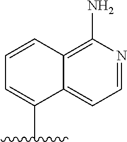 [SMILES: *c1cccc2c(N)nccc12] | O |
| 6644 | 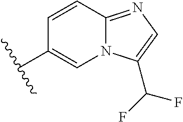 [SMILES: *c1ccc2ncc(C(F)F)n2c1] | OCH₂ | CH₂ | NH | 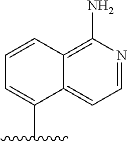 [SMILES: *c1cccc2c(N)nccc12] | O |
| 6645 | 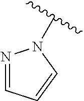 [SMILES: *n1cccn1] | CH₂ | CH₂ | NH | 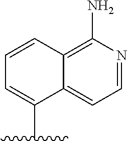 [SMILES: *c1cccc2c(N)nccc12] | O |
| 6646 | 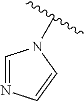 [SMILES: *n1ccnc1] | CH₂ | CH₂ | NH | 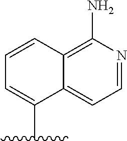 [SMILES: *c1cccc2c(N)nccc12] | O |
| 6647 | 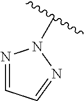 [SMILES: *n1nccn1] | CH₂ | CH₂ | NH | 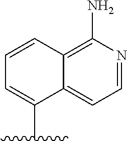 [SMILES: *c1cccc2c(N)nccc12] | O |
| 6648 | 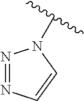 [SMILES: *n1ccnn1] | CH₂ | CH₂ | NH | 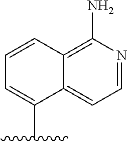 [SMILES: *c1cccc2c(N)nccc12] | O |
| 6649 | 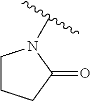 [SMILES: *N1CCCC1=O] | CH₂ | CH₂ | NH | 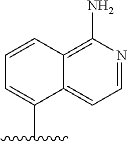 [SMILES: *c1cccc2c(N)nccc12] | O |
| 6650 | 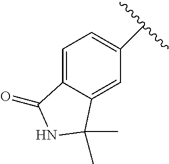 [SMILES: *c1ccc2c(c1)C(C)(C)NC2=O] | OCH₂ | CH₂ | NH | 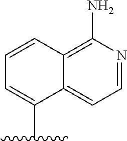 [SMILES: *c1cccc2c(N)nccc12] | O |
| 6651 | 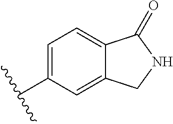 [SMILES: *c1ccc2c(c1)CNC2=O] | OCH₂ | CH₂ | NH | 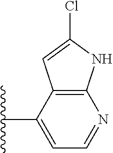 [SMILES: *c1ccnc2[nH]c(Cl)cc12] | O |
| 6653 | 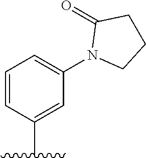 [SMILES: *c1cccc(N2CCCC2=O)c1] | OCH₂ | CH₂ | NH | 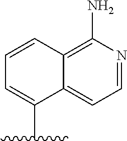 [SMILES: *c1cccc2c(N)nccc12] | O |
| 6654 | 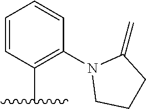 [SMILES: *c1ccccc1N1CCCC1=C] | OCH₂ | CH₂ | NH | 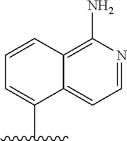 [SMILES: *c1cccc2c(N)nccc12] | O |
| 6656 | 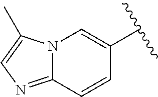 [SMILES: *c1ccc2ncc(C)n2c1] | OCH₂ | CH₂ | NH | 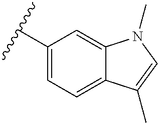 [SMILES: *c1ccc2c(C)cn(C)c2c1] | O |
| 6658 | 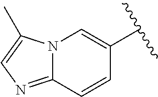 [SMILES: *c1ccc2ncc(C)n2c1] | OCH₂ | CH₂ | NH | 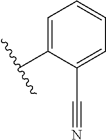 [SMILES: *c1ccccc1C#N] | O |
| 6659 | 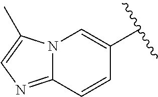 [SMILES: *c1ccc2ncc(C)n2c1] | OCH₂ | CH₂ | NH | 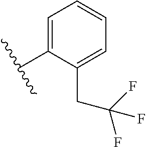 [SMILES: *c1ccccc1CC(F)(F)F] | O |
| 6660 | 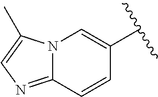 [SMILES: *c1ccc2ncc(C)n2c1] | OCH₂ | CH₂ | NH | 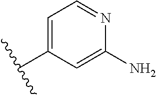 [SMILES: *c1ccnc(N)c1] | O |
| 6661 | 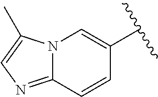 [SMILES: *c1ccc2ncc(C)n2c1] | OCH₂ | CH₂ | NH | 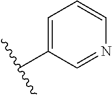 [SMILES: *c1cccnc1] | O |
| 6663 | 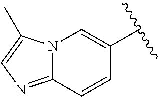 [SMILES: *c1ccc2ncc(C)n2c1] | OCH₂ | CH₂ | NH | 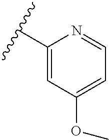 [SMILES: *c1cc(OC)ccn1] | O |

TABLE 7a

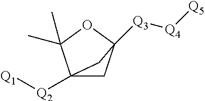

| Example | | | | | |
|---|---|---|---|---|---|
| No | Q₁ | Q₂ | Q₃ | Q₄ | Q₅ |
| 7701 | 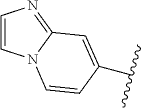 [SMILES: *c1ccn2ccnc2c1] | OCH₂ | CH₂ | NH | 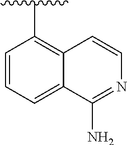 [SMILES: *c1cccc2c(N)nccc12] |
| 7702 | 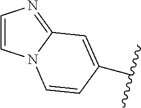 [SMILES: *c1ccn2ccnc2c1] | OCH₂ | CH₂ | O | 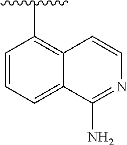 [SMILES: *c1cccc2c(N)nccc12] |

TABLE 7b

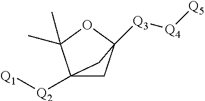

| Example | | | | | |
|---|---|---|---|---|---|
| Number | Q₁ | Q₂ | Q₃ | Q₄ | Q₅ |
| 7703 | 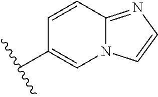 [SMILES: *c1ccc2nccn2c1] | OCH₂ | CH₂ | NH | 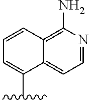 [SMILES: *c1cccc2c(N)nccc12] |
| 7704 | 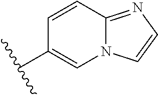 [SMILES: *c1ccc2nccn2c1] | OCH₂ | CH₂ | O | 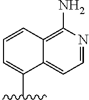 [SMILES: *c1cccc2c(N)nccc12] |

TABLE 8a

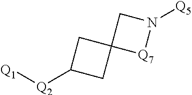

| Ex- | | | | |
|---|---|---|---|---|
| ample | | | | |
| No | Q₁ | Q₂ | Q₅ | Q₇ |
| 8801 | 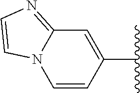 [SMILES: *c1ccn2ccnc2c1] | CH₂O | 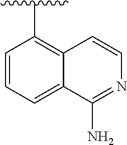 [SMILES: *c1cccc2c(N)nccc12] | CH₂ |
| 8802 | 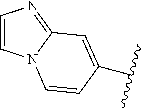 [SMILES: *c1ccn2ccnc2c1] | CH₂O | 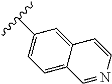 [SMILES: *c1ccc2cnccc2c1] | CH₂ |
| 8803 | 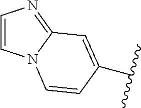 [SMILES: *c1ccn2ccnc2c1] | CH₂O | 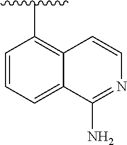 [SMILES: *c1cccc2c(N)nccc12] | CH₂CH₂ |

TABLE 8b

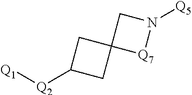

| Ex- | | | | |
|---|---|---|---|---|
| ample | | | | |
| No | Q₁ | Q₂ | Q₅ | Q₇ |
| 8804 | 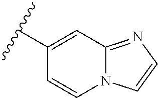 [SMILES: *c1ccn2ccnc2c1] | CH₂O | 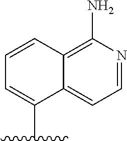 [SMILES: *c1cccc2c(N)nccc12] | CH₂CH₂ |
| 8805 | 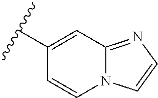 [SMILES: *c1ccn2ccnc2c1] | CH₂O | 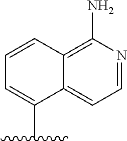 [SMILES: *c1cccc2c(N)nccc12] | CH₂CH₂ |
| 8806 | 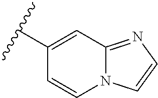 [SMILES: *c1ccn2ccnc2c1] | OCH₂ | 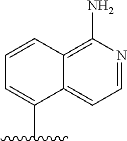 [SMILES: *c1cccc2c(N)nccc12] | CH₂CH₂ |
| 8807 | 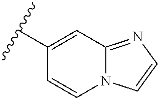 [SMILES: *c1ccn2ccnc2c1] | OCH₂ | 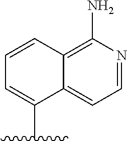 [SMILES: *c1cccc2c(N)nccc12] | CH₂CH₂ |
| 8808 | 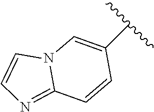 [SMILES: *c1ccc2nccn2c1] | OCH₂ | 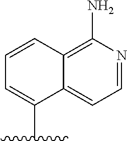 [SMILES: *c1cccc2c(N)nccc12] | CH₂ |

TABLE 9b

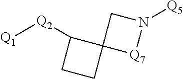

| Example | | | | |
|---|---|---|---|---|
| No | Q₁ | Q₂ | Q₅ | Q₇ |
| 9001 | 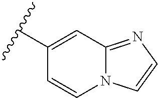 [SMILES: *c1ccn2ccnc2c1] | CH₂O | 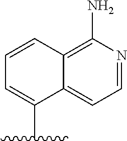 [SMILES: *c1cccc2c(N)nccc12] | CH₂ |
| 9002 | 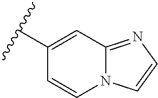 [SMILES: *c1ccn2ccnc2c1] | CH₂O | 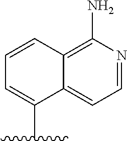 [SMILES: *c1cccc2c(N)nccc12] | CH₂ |
| 9003 | 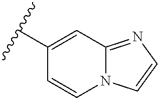 [SMILES: *c1ccn2ccnc2c1] | CH₂O | 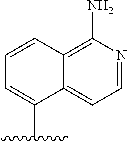 [SMILES: *c1cccc2c(N)nccc12] | CH₂ |

TABLE 10a

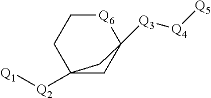

| Ex- | | | | | | |
|---|---|---|---|---|---|---|
| ample | | | | | | |
| No | Q₁ | Q₂ | Q₃ | Q₄ | Q₅ | Q₆ |
| 10901 | 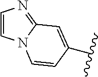 [SMILES: *c1ccn2ccnc2c1] | OCH₂ | CH₂ | NH | 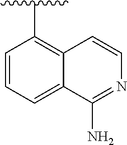 [SMILES: *c1cccc2c(N)nccc12] | O |
| 10902 | 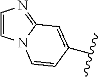 [SMILES: *c1ccn2ccnc2c1] | OCH₂ | CH₂ | NH | 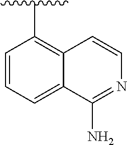 [SMILES: *c1cccc2c(N)nccc12] | CH₂ |
| 10903 | 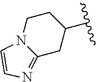 [SMILES: *C1CCn2ccnc2C1] | OCH₂ | CH₂ | NH | 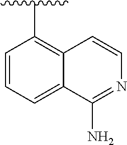 [SMILES: *c1cccc2c(N)nccc12] | O |
| 10904 | 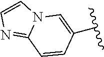 [SMILES: *c1ccc2nccn2c1] | OCH₂ | CH₂ | NH | 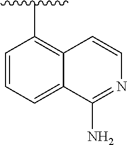 [SMILES: *c1cccc2c(N)nccc12] | O |

TABLE 10b

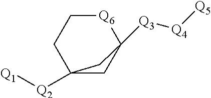

| Example | | | | | | |
|---|---|---|---|---|---|---|
| No | Q₁ | Q₂ | Q₃ | Q₄ | Q₅ | Q₆ |
| 10906 | 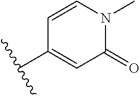 [SMILES: *c1ccn(C)c(=O)c1] | OCH₂ | CH₂ | NH | 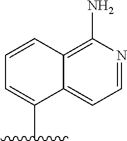 [SMILES: *c1cccc2c(N)nccc12] | O |
| 10907 | 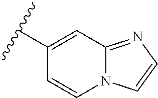 [SMILES: *c1ccn2ccnc2c1] | OCH₂ | CH₂ | NH | 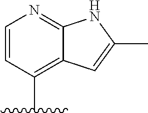 [SMILES: *c1ccnc2[nH]c(C)cc12] | O |
| 10908 | 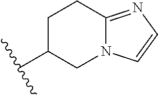 [SMILES: *C1CCc2nccn2C1] | OCH₂ | CH₂ | NH | 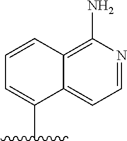 [SMILES: *c1cccc2c(N)nccc12] | O |
| 10909 | 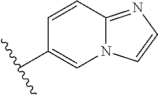 [SMILES: *c1ccc2nccn2c1] | OCH₂ | CH₂ | NH | 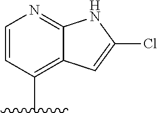 [SMILES: *c1ccnc2[nH]c(Cl)cc12] | O |
| 10910 | 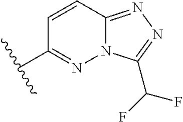 [SMILES: *c1ccc2nnc(C(F)F)n2n1] | OCH₂ | CH₂ | NH | 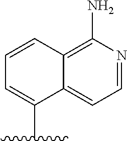 [SMILES: *c1cccc2c(N)nccc12] | O |
| 10911 | 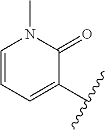 [SMILES: *c1cccn(C)c1=O] | OCH₂ | CH₂ | NH | 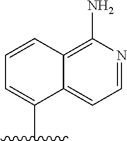 [SMILES: *c1cccc2c(N)nccc12] | O |
| 10912 | 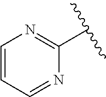 [SMILES: *c1ncccn1] | OCH₂ | CH₂ | NH | 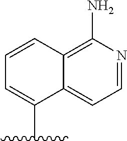 [SMILES: *c1cccc2c(N)nccc12] | O |
| 10914 | 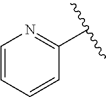 [SMILES: *c1ccccn1] | OCH₂ | CH₂ | NH | 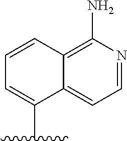 [SMILES: *c1cccc2c(N)nccc12] | O |
| 10915 | 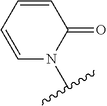 [SMILES: *n1ccccc1=O] | CH₂ | CH₂ | NH | 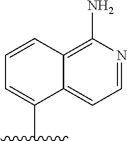 [SMILES: *c1cccc2c(N)nccc12] | O |
| 10918 | 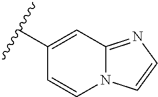 [SMILES: *c1ccn2ccnc2c1] | OCH₂ | CH₂ | NH | 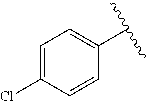 [SMILES: *c1ccc(Cl)cc1] | O |
| 10919 | 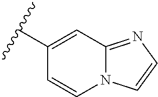 [SMILES: *c1ccn2ccnc2c1] | OCH₂ | CH₂ | NH | 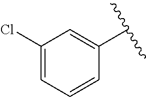 [SMILES: *c1cccc(Cl)c1] | O |
| 10920 | 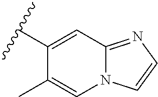 [SMILES: *c1cc2nccn2cc1C] | OCH₂ | CH₂ | NH | 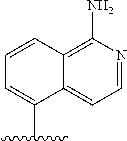 [SMILES: *c1cccc2c(N)nccc12] | O |
| 10921 | 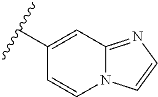 [SMILES: *c1ccn2ccnc2c1] | OCH₂ | CH₂ | NH | 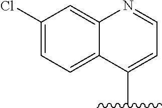 [SMILES: *c1ccnc2cc(Cl)ccc12] | O |
| 10922 | 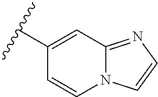 [SMILES: *c1ccn2ccnc2c1] | OCH₂ | CH₂ | NH | 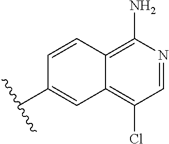 [SMILES: *c1ccc2c(N)ncc(Cl)c2c1] | O |
| 10923 | 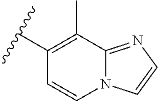 [SMILES: *c1ccn2ccnc2c1C] | OCH₂ | CH₂ | NH | 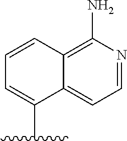 [SMILES: *c1cccc2c(N)nccc12] | O |
| 10924 | 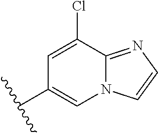 [SMILES: *c1cc(Cl)c2nccn2c1] | OCH₂ | CH₂ | NH | 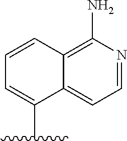 [SMILES: *c1cccc2c(N)nccc12] | O |
| 10925 | 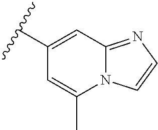 [SMILES: *c1cc(C)n2ccnc2c1] | OCH₂ | CH₂ | NH | 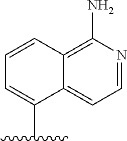 [SMILES: *c1cccc2c(N)nccc12] | O |
| 10926 | 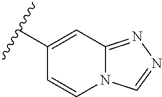 [SMILES: *c1ccn2cnnc2c1] | OCH₂ | CH₂ | NH | 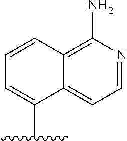 [SMILES: *c1cccc2c(N)nccc12] | O |
| 10928 | 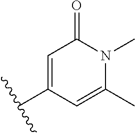 [SMILES: *c1cc(C)n(C)c(=O)c1] | OCH₂ | CH₂ | NH | 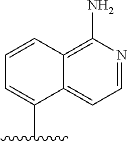 [SMILES: *c1cccc2c(N)nccc12] | O |
| 10929 | 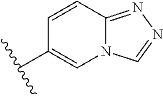 [SMILES: *c1ccc2nncn2c1] | OCH₂ | CH₂ | NH | 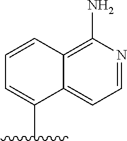 [SMILES: *c1cccc2c(N)nccc12] | O |
| 10930 | 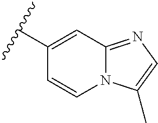 [SMILES: *c1ccn2c(C)cnc2c1] | OCH₂ | CH₂ | NH | 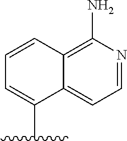 [SMILES: *c1cccc2c(N)nccc12] | O |
| 10931 | 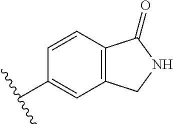 [SMILES: *c1ccc2c(c1)CNC2=O] | OCH₂ | CH₂ | NH | 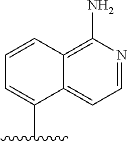 [SMILES: *c1cccc2c(N)nccc12] | O |
| 10932 | 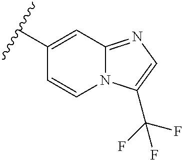 [SMILES: *c1ccn2c(C(F)(F)F)cnc2c1] | OCH₂ | CH₂ | NH | 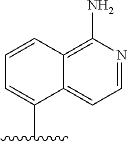 [SMILES: *c1cccc2c(N)nccc12] | O |
| 10933 | 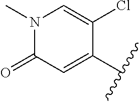 [SMILES: *c1cc(=O)n(C)cc1Cl] | OCH₂ | CH₂ | NH | 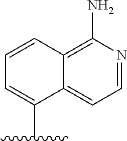 [SMILES: *c1cccc2c(N)nccc12] | O |
| 10934 | 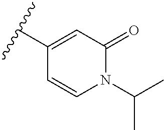 [SMILES: *c1ccn(C(C)C)c(=O)c1] | OCH₂ | CH₂ | NH | 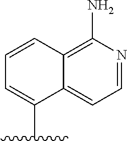 [SMILES: *c1cccc2c(N)nccc12] | O |
| 10936 | 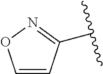 [SMILES: *c1ccon1] | OCH₂ | CH₂ | NH | 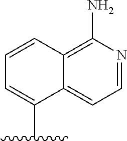 [SMILES: *c1cccc2c(N)nccc12] | O |
| 10937 | 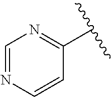 [SMILES: *c1ccncn1] | OCH₂ | CH₂ | NH | 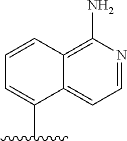 [SMILES: *c1cccc2c(N)nccc12] | O |
| 10938 | 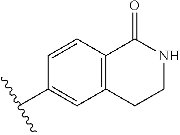 [SMILES: *c1ccc2c(c1)CCNC2=O] | OCH₂ | CH₂ | NH | 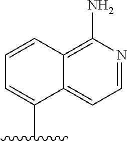 [SMILES: *c1cccc2c(N)nccc12] | O |
| 10939 | 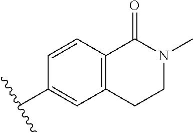 [SMILES: *c1ccc2c(c1)CCN(C)C2=O] | OCH₂ | CH₂ | NH | 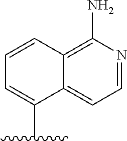 [SMILES: *c1cccc2c(N)nccc12] | O |
| 10940 | 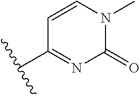 [SMILES: *c1ccn(C)c(=O)n1] | OCH₂ | CH₂ | NH | 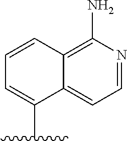 [SMILES: *c1cccc2c(N)nccc12] | O |
| 10941 | 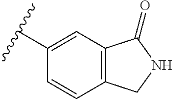 [SMILES: *c1ccc2c(c1)C(=O)NC2] | OCH₂ | CH₂ | NH | 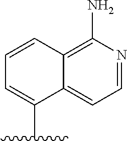 [SMILES: *c1cccc2c(N)nccc12] | O |
| 10942 | 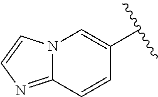 [SMILES: *c1ccc2nccn2c1] | OCH₂ | CH₂ | NH | 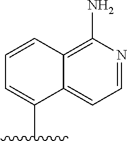 [SMILES: *c1cccc2c(N)nccc12] | CH₂ |
| 10943 | 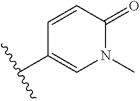 [SMILES: *c1ccc(=O)n(C)c1] | OCH₂ | CH₂ | NH | 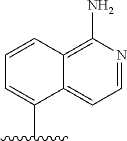 [SMILES: *c1cccc2c(N)nccc12] | O |
| 10944 | 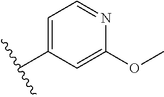 [SMILES: *c1ccnc(OC)c1] | OCH₂ | CH₂ | NH | 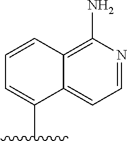 [SMILES: *c1cccc2c(N)nccc12] | O |
| 10945 | 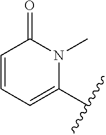 [SMILES: *c1cccc(=O)n1C] | OCH₂ | CH₂ | NH | 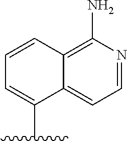 [SMILES: *c1cccc2c(N)nccc12] | O |
| 10946 | 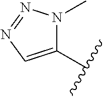 [SMILES: *c1cnnn1C] | OCH₂ | CH₂ | NH | 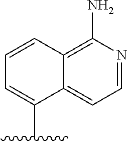 [SMILES: *c1cccc2c(N)nccc12] | O |
| 10948 | 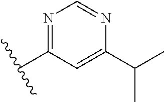 [SMILES: *c1cc(C(C)C)ncn1] | OCH₂ | CH₂ | NH | 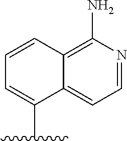 [SMILES: *c1cccc2c(N)nccc12] | O |
| 10949 | 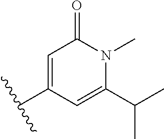 [SMILES: *c1cc(C(C)C)n(C)c(=O)c1] | OCH₂ | CH₂ | NH | 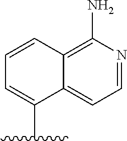 [SMILES: *c1cccc2c(N)nccc12] | O |
| 10951 | 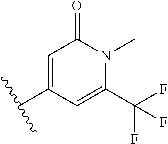 [SMILES: *c1cc(C(F)(F)F)n(C)c(=O)c1] | OCH₂ | CH₂ | NH | 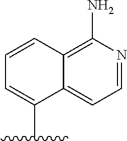 [SMILES: *c1cccc2c(N)nccc12] | O |
| 10952 | 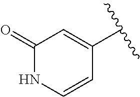 [SMILES: *c1cc[nH]c(=O)c1] | OCH₂ | CH₂ | NH | 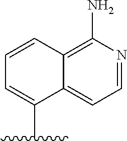 [SMILES: *c1cccc2c(N)nccc12] | O |
| 10953 | 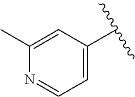 [SMILES: *c1ccnc(C)c1] | OCH₂ | CH₂ | NH | 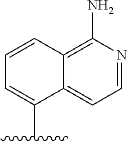 [SMILES: *c1cccc2c(N)nccc12] | O |
| 10954 | 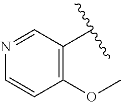 [SMILES: *c1cnccc1OC] | OCH₂ | CH₂ | NH | 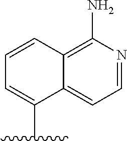 [SMILES: *c1cccc2c(N)nccc12] | O |
| 10955 | 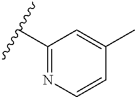 [SMILES: *c1cc(C)ccn1] | OCH₂ | CH₂ | NH | 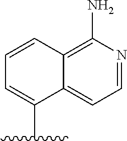 [SMILES: *c1cccc2c(N)nccc12] | O |
| 10956 | 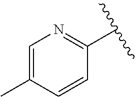 [SMILES: *c1ccc(C)cn1] | OCH₂ | CH₂ | NH | 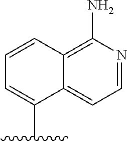 [SMILES: *c1cccc2c(N)nccc12] | O |
| 10957 | 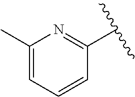 [SMILES: *c1cccc(C)n1] | OCH₂ | CH₂ | NH | 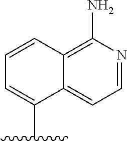 [SMILES: *c1cccc2c(N)nccc12] | O |
| 10958 | 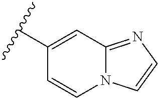 [SMILES: *c1ccn2ccnc2c1] | OCH₂ | CH₂ | NH | 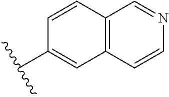 [SMILES: *c1ccc2cnccc2c1] | O |
| 10961 | 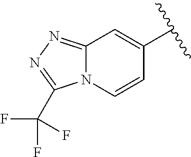 [SMILES: *c1ccn2c(C(F)(F)F)nnc2c1] | OCH₂ | CH₂ | NH | 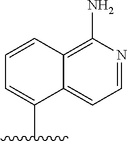 [SMILES: *c1cccc2c(N)nccc12] | O |
| 10962 | 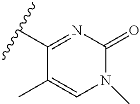 [SMILES: *c1nc(=O)n(C)cc1C] | OCH₂ | CH₂ | NH | 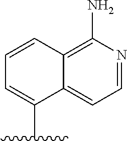 [SMILES: *c1cccc2c(N)nccc12] | O |
| 10963 | 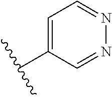 [SMILES: *c1ccnnc1] | OCH₂ | CH₂ | NH | 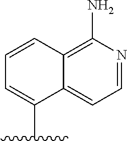 [SMILES: *c1cccc2c(N)nccc12] | O |
| 10964 | 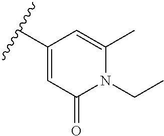 [SMILES: *c1cc(C)n(CC)c(=O)c1] | OCH₂ | CH₂ | NH | 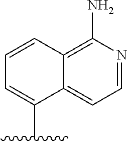 [SMILES: *c1cccc2c(N)nccc12] | O |
| 10965 | 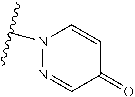 [SMILES: *n1ccc(=O)cn1] | CH₂ | CH₂ | NH | 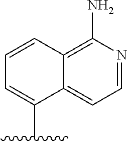 [SMILES: *c1cccc2c(N)nccc12] | O |
| 10966 | 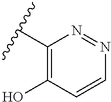 [SMILES: *c1nnccc1O] | CH₂ | CH₂ | NH | 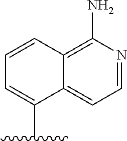 [SMILES: *c1cccc2c(N)nccc12] | O |
| 10968 | 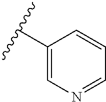 [SMILES: *c1cccnc1] | OCH₂ | CH₂ | NH | 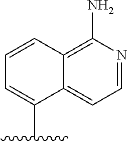 [SMILES: *c1cccc2c(N)nccc12] | O |
| 10969 | 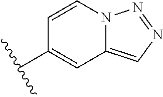 [SMILES: *c1ccn2nncc2c1] | OCH₂ | CH₂ | NH | 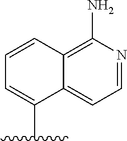 [SMILES: *c1cccc2c(N)nccc12] | O |
| 10970 | 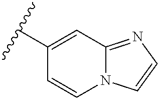 [SMILES: *c1ccn2ccnc2c1] | OCH₂ | CH₂ | NH | 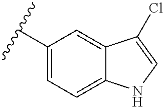 [SMILES: *c1ccc2[nH]cc(Cl)c2c1] | O |
| 10971 | 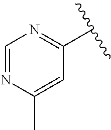 [SMILES: *c1cc(C)ncn1] | OCH₂ | CH₂ | NH | 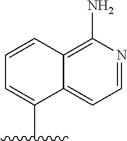 [SMILES: *c1cccc2c(N)nccc12] | O |
| 10972 | 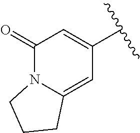 [SMILES: *c1cc2n(c(=O)c1)CCC2] | OCH₂ | CH₂ | NH | 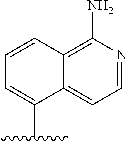 [SMILES: *c1cccc2c(N)nccc12] | O |
| 10973 | 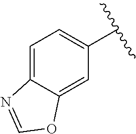 [SMILES: *c1ccc2ncoc2c1] | OCH₂ | CH₂ | NH | 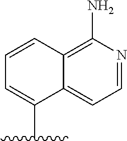 [SMILES: *c1cccc2c(N)nccc12] | O |
| 10974 | 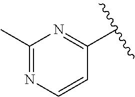 [SMILES: *c1ccnc(C)n1] | OCH₂ | CH₂ | NH | 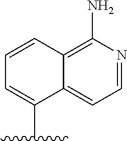 [SMILES: *c1cccc2c(N)nccc12] | O |
| 10975 | 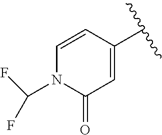 [SMILES: *c1ccn(C(F)F)c(=O)c1] | OCH₂ | CH₂ | NH | 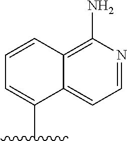 [SMILES: *c1cccc2c(N)nccc12] | O |
| 10976 | 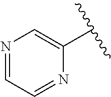 [SMILES: *c1cnccn1] | OCH₂ | CH₂ | NH | 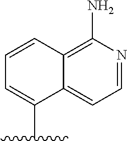 [SMILES: *c1cccc2c(N)nccc12] | O |

TABLE 11b

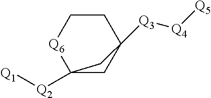

| Example | | | | | | |
|---|---|---|---|---|---|---|
| No | Q₁ | Q₂ | Q₃ | Q₄ | Q₅ | Q₆ |
| 11001 | 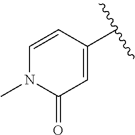 [SMILES: *c1ccn(C)c(=O)c1] | OCH₂ | CH₂ | NH | 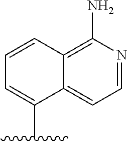 [SMILES: *c1cccc2c(N)nccc12] | O |

TABLE 12b

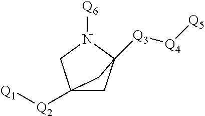

| Ex- | | | | | | |
|---|---|---|---|---|---|---|
| ample | | | | | | |
| No | Q₁ | Q₂ | Q₃ | Q₄ | Q₅ | Q₆ |
| 12001 | 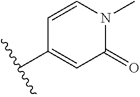 [SMILES: *c1ccn(C)c(=O)c1] | OCH₂ | CH₂ | NH | 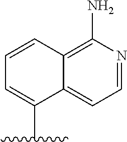 [SMILES: *c1cccc2c(N)nccc12] | 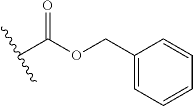 [SMILES: *C(=O)OCc1ccccc1] |
| 12007 | 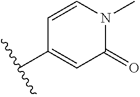 [SMILES: *c1ccn(C)c(=O)c1] | OCH₂ | CH₂ | NH | 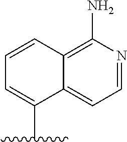 [SMILES: *c1cccc2c(N)nccc12] | H |
| 12008 | 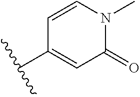 [SMILES: *c1ccn(C)c(=O)c1] | OCH₂ | CH₂ | NH | 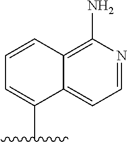 [SMILES: *c1cccc2c(N)nccc12] | 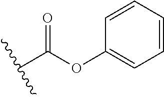 [SMILES: *C(=O)Oc1ccccc1] |
| 12009 | 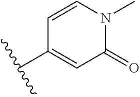 [SMILES: *c1ccn(C)c(=O)c1] | OCH₂ | CH₂ | NH | 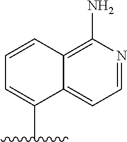 [SMILES: *c1cccc2c(N)nccc12] | 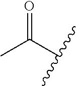 [SMILES: *C(C)=O] |
| 12010 | 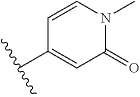 [SMILES: *c1ccn(C)c(=O)c1] | OCH₂ | CH₂ | NH | 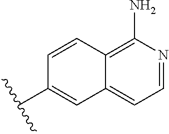 [SMILES: *c1ccc2c(N)nccc2c1] | 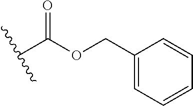 [SMILES: *C(=O)OCc1ccccc1] |
| 12011 | 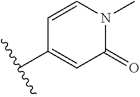 [SMILES: *c1ccn(C)c(=O)c1] | OCH₂ | CH₂ | NH | 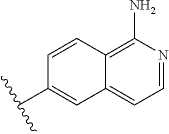 [SMILES: *c1ccc2c(N)nccc2c1] | H |
| 12012 | 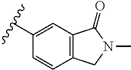 [SMILES: *c1ccc2c(c1)C(=O)N(C)C2] | OCH₂ | CH₂ | NH | 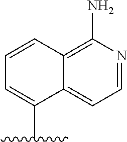 [SMILES: *c1cccc2c(N)nccc12] | 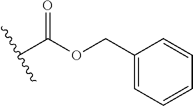 [SMILES: *C(=O)OCc1ccccc1] |
| 12013 | 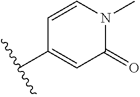 [SMILES: *c1ccn(C)c(=O)c1] | OCH₂ | CH₂ | NH | 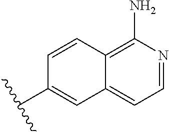 [SMILES: *c1ccc2c(N)nccc2c1] | 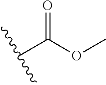 [SMILES: *C(=O)OC] |
| 12014 | 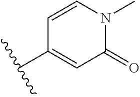 [SMILES: *c1ccn(C)c(=O)c1] | OCH₂ | CH₂ | NH | 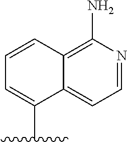 [SMILES: *c1cccc2c(N)nccc12] | 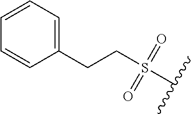 [SMILES: *S(=O)(=O)CCc1ccccc1] |
| 12015 | 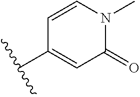 [SMILES: *c1ccn(C)c(=O)c1] | OCH₂ | CH₂ | NH | 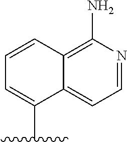 [SMILES: *c1cccc2c(N)nccc12] | 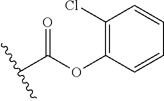 [SMILES: *C(=O)Oc1ccccc1Cl] |
| 12016 | 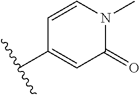 [SMILES: *c1ccn(C)c(=O)c1] | OCH₂ | CH₂ | NH | 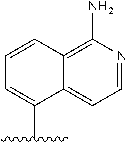 [SMILES: *c1cccc2c(N)nccc12] | 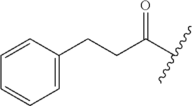 [SMILES: *C(=O)CCc1ccccc1] |
| 12017 | 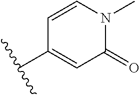 [SMILES: *c1ccn(C)c(=O)c1] | OCH₂ | CH₂ | NH | 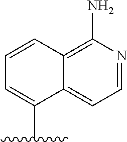 [SMILES: *c1cccc2c(N)nccc12] | 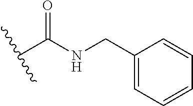 [SMILES: *C(=O)NCc1ccccc1] |
| 12018 | 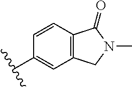 [SMILES: *c1ccc2c(c1)CN(C)C2=O] | OCH₂ | CH₂ | NH | 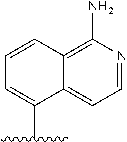 [SMILES: *c1cccc2c(N)nccc12] | 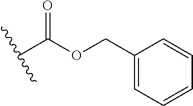 [SMILES: *C(=O)OCc1ccccc1] |
| 12019 | 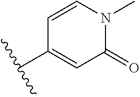 [SMILES: *c1ccn(C)c(=O)c1] | OCH₂ | CH₂ | NH | 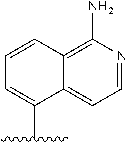 [SMILES: *c1cccc2c(N)nccc12] | 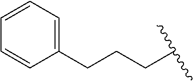 [SMILES: *CCCc1ccccc1] |
| 12020 | 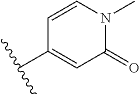 [SMILES: *c1ccn(C)c(=O)c1] | OCH₂ | CH₂ | NH | 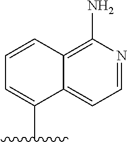 [SMILES: *c1cccc2c(N)nccc12] | 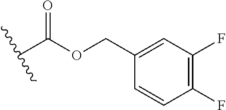 [SMILES: *C(=O)OCc1ccc(F)c(F)c1] |
| 12021 | 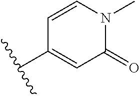 [SMILES: *c1ccn(C)c(=O)c1] | OCH₂ | CH₂ | NH | 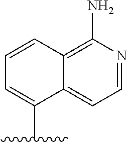 [SMILES: *c1cccc2c(N)nccc12] | 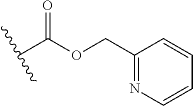 [SMILES: *C(=O)OCc1ccccn1] |
| 12022 | 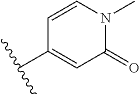 [SMILES: *c1ccn(C)c(=O)c1] | OCH₂ | CH₂ | NH | 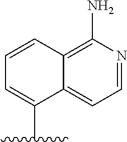 [SMILES: *c1cccc2c(N)nccc12] | 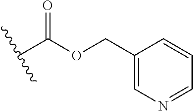 [SMILES: *C(=O)OCc1cccnc1] |
| 12023 | 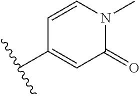 [SMILES: *c1ccn(C)c(=O)c1] | OCH₂ | CH₂ | NH | 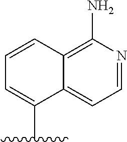 [SMILES: *c1cccc2c(N)nccc12] | 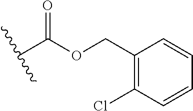 [SMILES: *C(=O)OCc1ccccc1Cl] |
| 12024 | 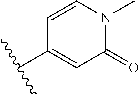 [SMILES: *c1ccn(C)c(=O)c1] | OCH₂ | CH₂ | NH | 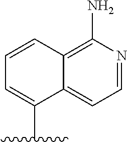 [SMILES: *c1cccc2c(N)nccc12] | 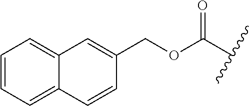 [SMILES: *C(=O)OCc1ccc2ccccc2c1] |
| 12025 | 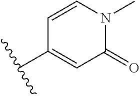 [SMILES: *c1ccn(C)c(=O)c1] | OCH₂ | CH₂ | NH | 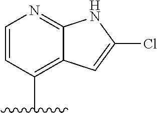 [SMILES: *c1ccnc2[nH]c(Cl)cc12] | 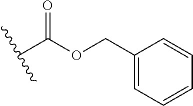 [SMILES: *C(=O)OCc1ccccc1] |
| 12026 | 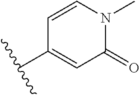 [SMILES: *c1ccn(C)c(=O)c1] | OCH₂ | CH₂ | NH | 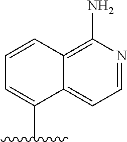 [SMILES: *c1cccc2c(N)nccc12] | 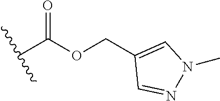 [SMILES: *C(=O)OCc1cnn(C)c1] |
| 12027 | 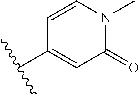 [SMILES: *c1ccn(C)c(=O)c1] | OCH₂ | CH₂ | NH | 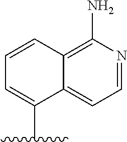 [SMILES: *c1cccc2c(N)nccc12] | 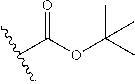 [SMILES: *C(=O)OC(C)(C)C] |
| 12028 | 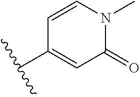 [SMILES: *c1ccn(C)c(=O)c1] | OCH₂ | CH₂ | NH | 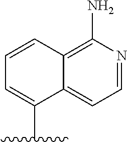 [SMILES: *c1cccc2c(N)nccc12] | 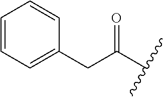 [SMILES: *C(=O)Cc1ccccc1] |
| 12031 | 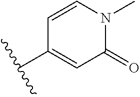 [SMILES: *c1ccn(C)c(=O)c1] | OCH₂ | CH₂ | NH | 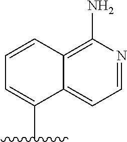 [SMILES: *c1cccc2c(N)nccc12] | 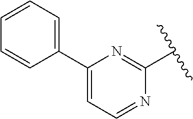 [SMILES: *c1nccc(-c2ccccc2)n1] |
| 12034 | 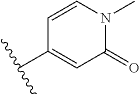 [SMILES: *c1ccn(C)c(=O)c1] | OCH₂ | CH₂ | NH | 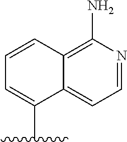 [SMILES: *c1cccc2c(N)nccc12] | 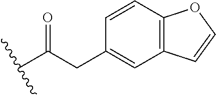 [SMILES: *C(=O)Cc1ccc2occc2c1] |
| 12035 | 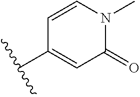 [SMILES: *c1ccn(C)c(=O)c1] | OCH₂ | CH₂ | NH | 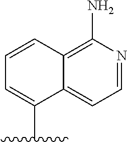 [SMILES: *c1cccc2c(N)nccc12] | 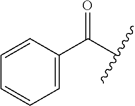 [SMILES: *C(=O)c1ccccc1] |
| 12036 | 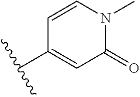 [SMILES: *c1ccn(C)c(=O)c1] | OCH₂ | CH₂ | NH | 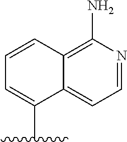 [SMILES: *c1cccc2c(N)nccc12] | 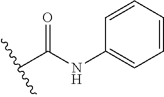 [SMILES: *C(=O)Nc1ccccc1] |
| 12038 | 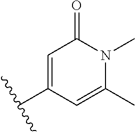 [SMILES: *c1cc(C)n(C)c(=O)c1] | OCH₂ | CH₂ | NH | 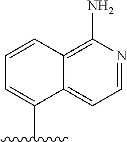 [SMILES: *c1cccc2c(N)nccc12] | 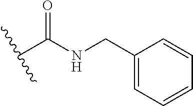 [SMILES: *C(=O)NCc1ccccc1] |
| 12039 | 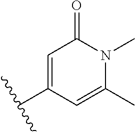 [SMILES: *c1cc(C)n(C)c(=O)c1] | OCH₂ | CH₂ | NH | 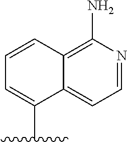 [SMILES: *c1cccc2c(N)nccc12] | 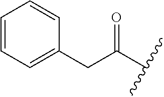 [SMILES: *C(=O)Cc1ccccc1] |
| 12040 | 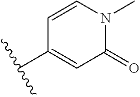 [SMILES: *c1ccn(C)c(=O)c1] | OCH₂ | CH₂ | NH | 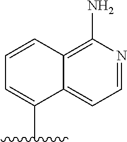 [SMILES: *c1cccc2c(N)nccc12] | 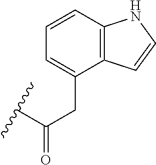 [SMILES: *C(=O)Cc1cccc2[nH]ccc12] |
| 12041 | 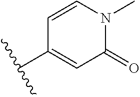 [SMILES: *c1ccn(C)c(=O)c1] | OCH₂ | CH₂ | NH | 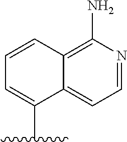 [SMILES: *c1cccc2c(N)nccc12] | 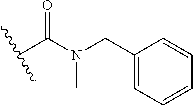 [SMILES: *C(=O)N(C)Cc1ccccc1] |
| 12042 | 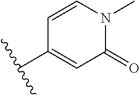 [SMILES: *c1ccn(C)c(=O)c1] | OCH₂ | CH₂ | NH | 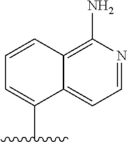 [SMILES: *c1cccc2c(N)nccc12] | 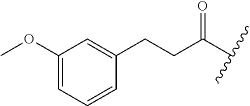 [SMILES: *C(=O)CCc1cccc(OC)c1] |
| 12044 | 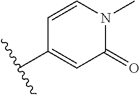 [SMILES: *c1ccn(C)c(=O)c1] | OCH₂ | CH₂ | NH | 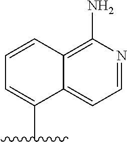 [SMILES: *c1cccc2c(N)nccc12] | 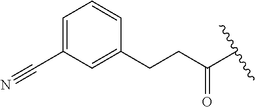 [SMILES: *C(=O)CCc1cccc(C#N)c1] |
| 12045 | 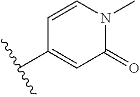 [SMILES: *c1ccn(C)c(=O)c1] | OCH₂ | CH₂ | NH | 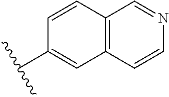 [SMILES: *c1ccc2cnccc2c1] | 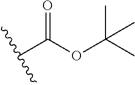 [SMILES: *C(=O)OC(C)(C)C] |
| 12046 | 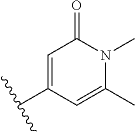 [SMILES: *c1cc(C)n(C)c(=O)c1] | OCH₂ | CH₂ | NH | 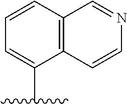 [SMILES: *c1cccc2cnccc12] | 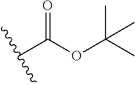 [SMILES: *C(=O)OC(C)(C)C] |
| 12047 | 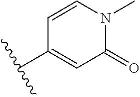 [SMILES: *c1ccn(C)c(=O)c1] | OCH₂ | CH₂ | NH | 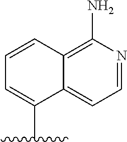 [SMILES: *c1cccc2c(N)nccc12] | 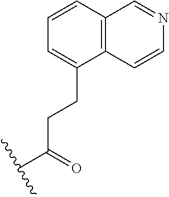 [SMILES: *C(=O)CCc1cccc2cnccc12] |
| 12048 | 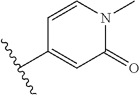 [SMILES: *c1ccn(C)c(=O)c1] | OCH₂ | CH₂ | NH | 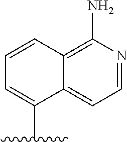 [SMILES: *c1cccc2c(N)nccc12] | 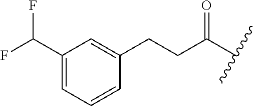 [SMILES: *C(=O)CCc1cccc(C(F)F)c1] |
| 12049 | 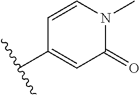 [SMILES: *c1ccn(C)c(=O)c1] | OCH₂ | CH₂ | NH | 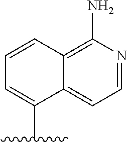 [SMILES: *c1cccc2c(N)nccc12] | 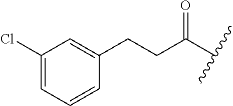 [SMILES: *C(=O)CCc1cccc(Cl)c1] |
| 12050 | 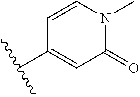 [SMILES: *c1ccn(C)c(=O)c1] | OCH₂ | CH₂ | NH | 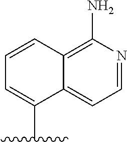 [SMILES: *c1cccc2c(N)nccc12] | 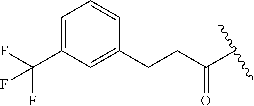 [SMILES: *C(=O)CCc1cccc(C(F)(F)F)c1] |
| 12051 | 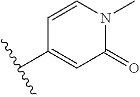 [SMILES: *c1ccn(C)c(=O)c1] | OCH₂ | CH₂ | NH | 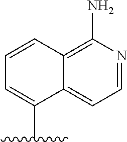 [SMILES: *c1cccc2c(N)nccc12] | 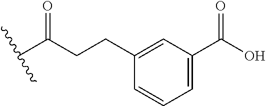 [SMILES: *C(=O)CCc1cccc(C(=O)O)c1] |
| 12052 | 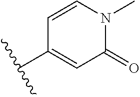 [SMILES: *c1ccn(C)c(=O)c1] | OCH₂ | CH₂ | NH | 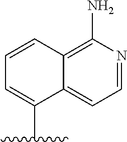 [SMILES: *c1cccc2c(N)nccc12] | 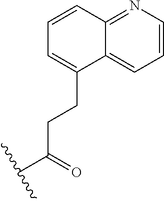 [SMILES: *C(=O)CCc1cccc2ncccc12] |
| 12053 | 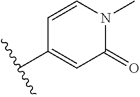 [SMILES: *c1ccn(C)c(=O)c1] | OCH₂ | CH₂ | NH | 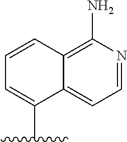 [SMILES: *c1cccc2c(N)nccc12] | 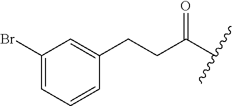 [SMILES: *C(=O)CCc1cccc(Br)c1] |
| 12054 | 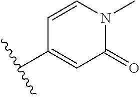 [SMILES: *c1ccn(C)c(=O)c1] | OCH₂ | CH₂ | NH | 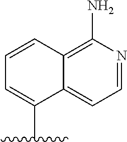 [SMILES: *c1cccc2c(N)nccc12] | 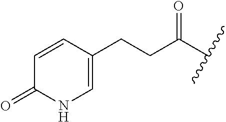 [SMILES: *C(=O)CCc1ccc(=O)[nH]c1] |
| 12055 | 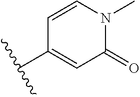 [SMILES: *c1ccn(C)c(=O)c1] | OCH₂ | CH₂ | NH | 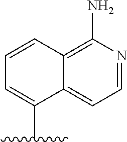 [SMILES: *c1cccc2c(N)nccc12] | 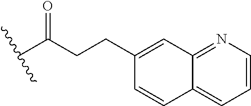 [SMILES: *C(=O)CCc1ccc2cccnc2c1] |
| 12056 | 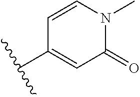 [SMILES: *c1ccn(C)c(=O)c1] | OCH₂ | CH₂ | NH | 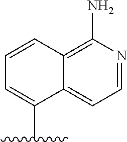 [SMILES: *c1cccc2c(N)nccc12] | 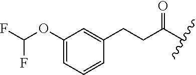 [SMILES: *C(=O)CCc1cccc(OC(F)F)c1] |
| 12057 | 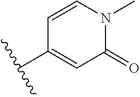 [SMILES: *c1ccn(C)c(=O)c1] | OCH₂ | CH₂ | NH | 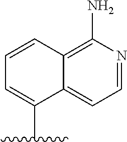 [SMILES: *c1cccc2c(N)nccc12] | 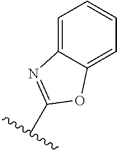 [SMILES: *c1nc2ccccc2o1] |
| 12058 | 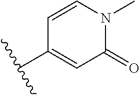 [SMILES: *c1ccn(C)c(=O)c1] | OCH₂ | CH₂ | NH | 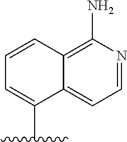 [SMILES: *c1cccc2c(N)nccc12] | 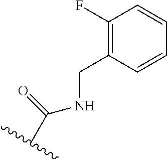 [SMILES: *C(=O)NCc1ccccc1F] |
| 12059 | 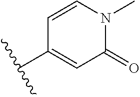 [SMILES: *c1ccn(C)c(=O)c1] | OCH₂ | CH₂ | NH | 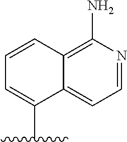 [SMILES: *c1cccc2c(N)nccc12] | 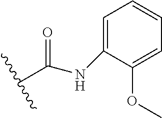 [SMILES: *C(=O)Nc1ccccc1OC] |
| 12060 | 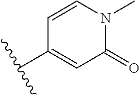 [SMILES: *c1ccn(C)c(=O)c1] | OCH₂ | CH₂ | NH | 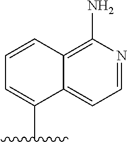 [SMILES: *c1cccc2c(N)nccc12] | 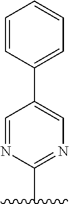 [SMILES: *c1ncc(-c2ccccc2)cn1] |
| 12061 | 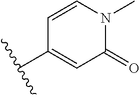 [SMILES: *c1ccn(C)c(=O)c1] | OCH₂ | CH₂ | NH | 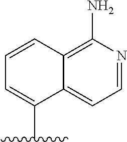 [SMILES: *c1cccc2c(N)nccc12] | 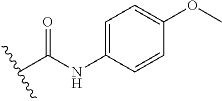 [SMILES: *C(=O)Nc1ccc(OC)cc1] |
| 12062 | 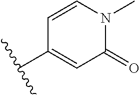 [SMILES: *c1ccn(C)c(=O)c1] | OCH₂ | CH₂ | NH | 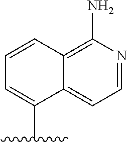 [SMILES: *c1cccc2c(N)nccc12] | 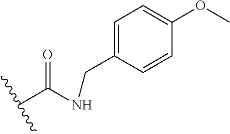 [SMILES: *C(=O)NCc1ccc(OC)cc1] |
| 12063 | 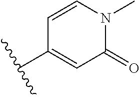 [SMILES: *c1ccn(C)c(=O)c1] | OCH₂ | CH₂ | NH | 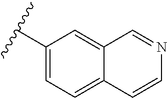 [SMILES: *c1ccc2ccncc2c1] | 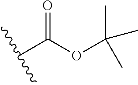 [SMILES: *C(=O)OC(C)(C)C] |
| 12064 | 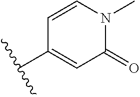 [SMILES: *c1ccn(C)c(=O)c1] | OCH₂ | CH₂ | NH | 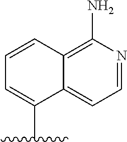 [SMILES: *c1cccc2c(N)nccc12] | 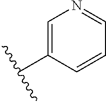 [SMILES: *c1cccnc1] |
| 12065 | 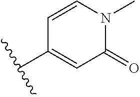 [SMILES: *c1ccn(C)c(=O)c1] | OCH₂ | CH₂ | NH | 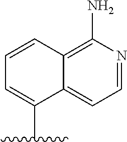 [SMILES: *c1cccc2c(N)nccc12] | 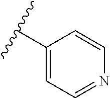 [SMILES: *c1ccncc1] |
| 12067 | 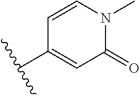 [SMILES: *c1ccn(C)c(=O)c1] | OCH₂ | CH₂ | NH | 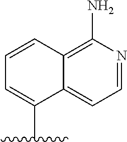 [SMILES: *c1cccc2c(N)nccc12] | 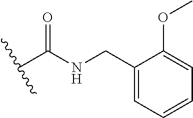 [SMILES: *C(=O)NCc1ccccc1OC] |
| 12068 | 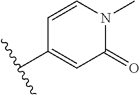 [SMILES: *c1ccn(C)c(=O)c1] | OCH₂ | CH₂ | NH | 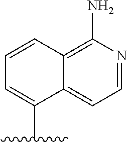 [SMILES: *c1cccc2c(N)nccc12] | 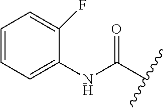 [SMILES: *C(=O)Nc1ccccc1F] |
| 12069 | 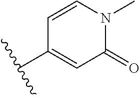 [SMILES: *c1ccn(C)c(=O)c1] | OCH₂ | CH₂ | NH | 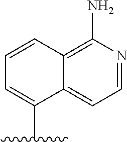 [SMILES: *c1cccc2c(N)nccc12] | 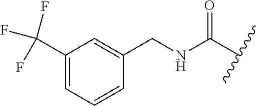 [SMILES: *C(=O)NCc1cccc(C(F)(F)F)c1] |
| 12070 | 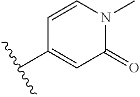 [SMILES: *c1ccn(C)c(=O)c1] | OCH₂ | CH₂ | NH | 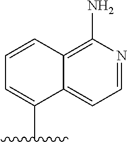 [SMILES: *c1cccc2c(N)nccc12] | 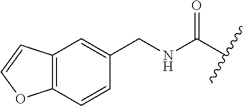 [SMILES: *C(=O)NCc1ccc2occc2c1] |
| 12071 | 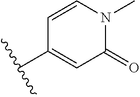 [SMILES: *c1ccn(C)c(=O)c1] | OCH₂ | CH₂ | NH | 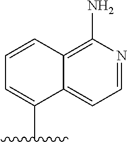 [SMILES: *c1cccc2c(N)nccc12] | 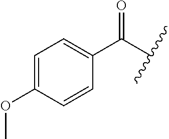 [SMILES: *C(=O)c1ccc(OC)cc1] |
| 12072 | 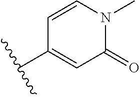 [SMILES: *c1ccn(C)c(=O)c1] | OCH₂ | CH₂ | NH | 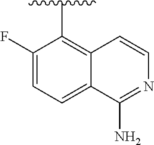 [SMILES: *c1c(F)ccc2c(N)nccc12] | 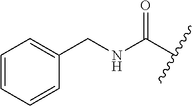 [SMILES: *C(=O)NCc1ccccc1] |

TABLE 13b

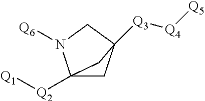

| Example | | | | | | |
|---|---|---|---|---|---|---|
| No | Q₁ | Q₂ | Q₃ | Q₄ | Q₅ | Q₆ |
| 13001 | 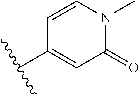 [SMILES: *c1ccn(C)c(=O)c1] | OCH₂ | CH₂ | NH | 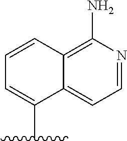 [SMILES: *c1cccc2c(N)nccc12] | 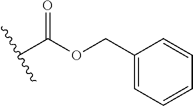 [SMILES: *C(=O)OCc1ccccc1] |
| 13002 | 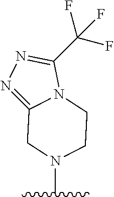 [SMILES: *N1CCn2c(nnc2C(F)(F)F)C1] | C═O | CH₂ | NH | 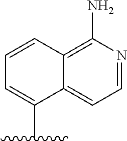 [SMILES: *c1cccc2c(N)nccc12] | 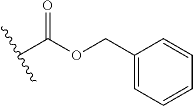 [SMILES: *C(=O)OCc1ccccc1] |
| 13003 | 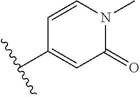 [SMILES: *c1ccn(C)c(=O)c1] | OCH₂ | CH₂ | NH | 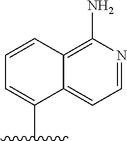 [SMILES: *c1cccc2c(N)nccc12] | 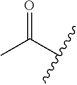 [SMILES: *C(C)=O] |
| 13004 | 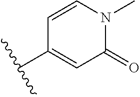 [SMILES: *c1ccn(C)c(=O)c1] | OCH₂ | CH₂ | NH | 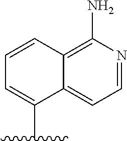 [SMILES: *c1cccc2c(N)nccc12] | 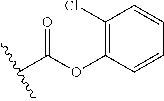 [SMILES: *C(=O)Oc1ccccc1Cl] |
| 13005 | 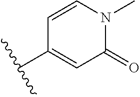 [SMILES: *c1ccn(C)c(=O)c1] | OCH₂ | CH₂ | NH | 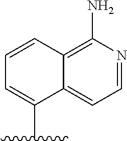 [SMILES: *c1cccc2c(N)nccc12] | 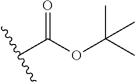 [SMILES: *C(=O)OC(C)(C)C] |
| 13006 | 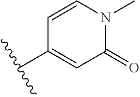 [SMILES: *c1ccn(C)c(=O)c1] | OCH₂ | CH₂ | NH | 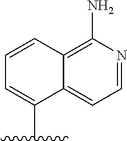 [SMILES: *c1cccc2c(N)nccc12] | 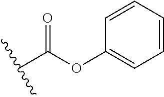 [SMILES: *C(=O)Oc1ccccc1] |
| 13007 | 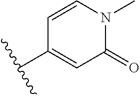 [SMILES: *c1ccn(C)c(=O)c1] | OCH₂ | CH₂ | NH | 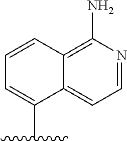 [SMILES: *c1cccc2c(N)nccc12] | H |
| 13008 | 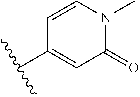 [SMILES: *c1ccn(C)c(=O)c1] | OCH₂ | CH₂ | NH | 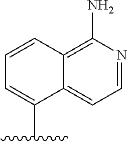 [SMILES: *c1cccc2c(N)nccc12] | 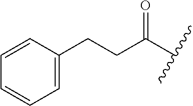 [SMILES: *C(=O)CCc1ccccc1] |
| 13009 | 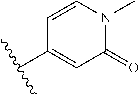 [SMILES: *c1ccn(C)c(=O)c1] | OCH₂ | CH₂ | NH | 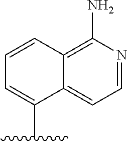 [SMILES: *c1cccc2c(N)nccc12] | 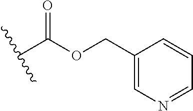 [SMILES: *C(=O)OCc1cccnc1] |
| 13010 | 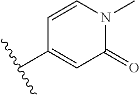 [SMILES: *c1ccn(C)c(=O)c1] | OCH₂ | CH₂ | NH | 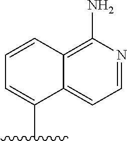 [SMILES: *c1cccc2c(N)nccc12] | 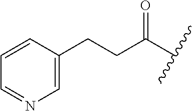 [SMILES: *C(=O)CCc1cccnc1] |
| 13011 | 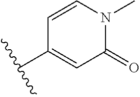 [SMILES: *c1ccn(C)c(=O)c1] | OCH₂ | CH₂ | NH | 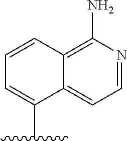 [SMILES: *c1cccc2c(N)nccc12] | 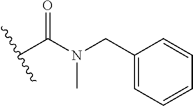 [SMILES: *C(=O)N(C)Cc1ccccc1] |
| 13012 | 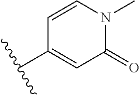 [SMILES: *c1ccn(C)c(=O)c1] | OCH₂ | CH₂ | NH | 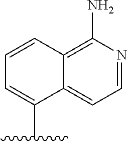 [SMILES: *c1cccc2c(N)nccc12] | 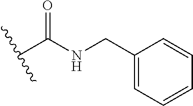 [SMILES: *C(=O)NCc1ccccc1] |
| 13013 | 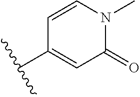 [SMILES: *c1ccn(C)c(=O)c1] | OCH₂ | CH₂ | NH | 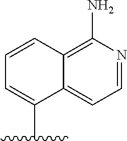 [SMILES: *c1cccc2c(N)nccc12] | 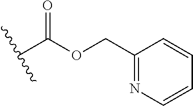 [SMILES: *C(=O)OCc1ccccn1] |
| 13014 | 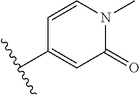 [SMILES: *c1ccn(C)c(=O)c1] | OCH₂ | CH₂ | NH | 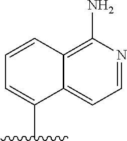 [SMILES: *c1cccc2c(N)nccc12] | 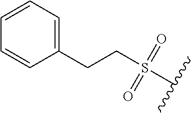 [SMILES: *S(=O)(=O)CCc1ccccc1] |
| 13015 | 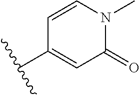 [SMILES: *c1ccn(C)c(=O)c1] | OCH₂ | CH₂ | NH | 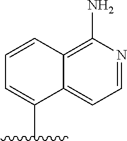 [SMILES: *c1cccc2c(N)nccc12] | 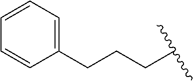 [SMILES: *CCCc1ccccc1] |
| 13016 | 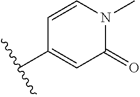 [SMILES: *c1ccn(C)c(=O)c1] | OCH₂ | CH₂ | NH | 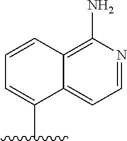 [SMILES: *c1cccc2c(N)nccc12] | 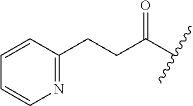 [SMILES: *C(=O)CCc1ccccn1] |
| 13017 | 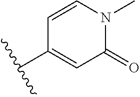 [SMILES: *c1ccn(C)c(=O)c1] | OCH₂ | CH₂ | NH | 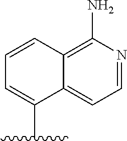 [SMILES: *c1cccc2c(N)nccc12] | 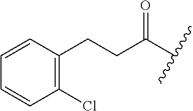 [SMILES: *C(=O)CCc1ccccc1Cl] |
| 13018 | 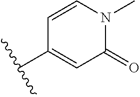 [SMILES: *c1ccn(C)c(=O)c1] | OCH₂ | CH₂ | NH | 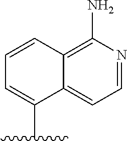 [SMILES: *c1cccc2c(N)nccc12] | 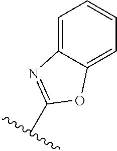 [SMILES: *c1nc2ccccc2o1] |
| 13019 | 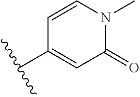 [SMILES: *c1ccn(C)c(=O)c1] | OCH₂ | CH₂ | NH | 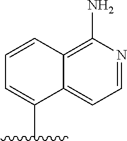 [SMILES: *c1cccc2c(N)nccc12] | 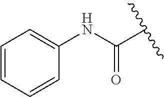 [SMILES: *C(=O)Nc1ccccc1] |
| 13020 | 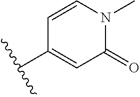 [SMILES: *c1ccn(C)c(=O)c1] | OCH₂ | CH₂ | NH | 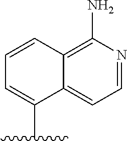 [SMILES: *c1cccc2c(N)nccc12] | 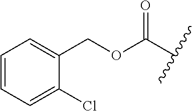 [SMILES: *C(=O)OCc1ccccc1Cl] |
| 13021 | 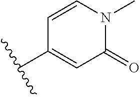 [SMILES: *c1ccn(C)c(=O)c1] | OCH₂ | CH₂ | NH | 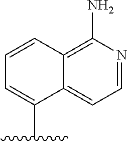 [SMILES: *c1cccc2c(N)nccc12] | 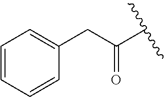 [SMILES: *C(=O)Cc1ccccc1] |
| 13022 | 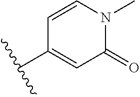 [SMILES: *c1ccn(C)c(=O)c1] | OCH₂ | CH₂ | NH | 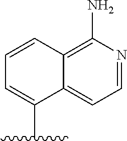 [SMILES: *c1cccc2c(N)nccc12] | 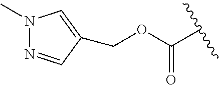 [SMILES: *C(=O)OCc1cnn(C)c1] |

Preferably, the compound of formula (I) is a compound selected from example numbers: 1001, 1002, 1002.1, 1002.2, 1004, 1005.1, 1006, 1009, 1010, 1011, 1012, 1013, 1014, 1015, 1016, 1017, 1018, 1019, 1101, 1105, 1109, 1110, 1113, 1118, 1125, 1126, 1127, 1129, 1130, 1131, 1132, 1133, 1134, 1135, 2020, 2022, 2204, 2205, 2206, 2207, 2208, 2210, 2211, 2212, 4401, 5003, 6601, 6602, 6605, 6606, 6608, 6609, 6610, 6611, 6612, 6613, 6614, 6615, 6616, 6617, 6618, 6619, 6620, 6621, 6622, 6623, 6624, 6625, 6626, 6627, 6628, 6629, 6630, 6631, 6633, 6634, 6635, 6636, 6637, 6638, 6639, 6640, 6641, 6642, 6643, 6644, 6645, 6646, 6647, 6648, 6649, 6650, 6651, 6653, 6654, 6656, 6658, 6659, 6660, 6661, 6663, 7702, 7703, 7704, 8801, 8803, 8804, 8805, 8806, 8807, 8808, 9001, 9002, 9003, 10901, 10903, 10904, 10906, 10907, 10908, 10909, 10910, 10911, 10912, 10914, 10915, 10918, 10919, 10920, 10921, 10922, 10923, 10924, 10925, 10926, 10928, 10929, 10930, 10931, 10932, 10933, 10934, 10936, 10937, 10938, 10939, 10940, 10941, 10942, 10943, 10944, 10945, 10946, 10948, 10949, 10951, 10952, 10953, 10954, 10955, 10956, 10957, 10958, 10961, 10962, 10963, 10964, 10965, 10966, 10968, 10969, 10970, 10971, 10972, 10973, 10974, 10975, 10976, 11001, 12001, 12007, 12008, 12009, 12010, 12011, 12012, 12013, 12014, 12015, 12016, 12017, 12018, 12019, 12020, 12021, 12022, 12023, 12024, 12025, 12026, 12027, 12028, 12031, 12034, 12035, 12036, 12038, 12039, 12040, 12041, 12042, 12044, 12045, 12046, 12047, 12048, 12049, 12050, 12051, 12052, 12053, 12054, 12055, 12056, 12057, 12058, 12059, 12060, 12061, 12062, 12063, 12064, 12065, 12067, 12068, 12069, 12070, 12071, 12072, 13001, 13002, 13003, 13004, 13005, 13006, 13007, 13008, 13009, 13010, 13011, 13012, 13013, 13014, 13015, 13016, 13017, 13018, 13019, 13020, 13021, and 13022, and pharmaceutically acceptable salts and/or solvates thereof. More preferably, the compound of formula (I) is a compound selected from example numbers: 12036, 12038, 12041, 12057, 12060, 12061, 12065, 12068, 2020, 2212, 6601, 6602, 6617, 6618, 6622, 6624, 6626, 6629, 6639, 10901, 10904, 10925, 10926, 10928, 10930, 10931, 10964, 10972, 12001, 12007, 12008, 12009, 12014, 12015, 12016, 12017, 12018, 12019, 12021, 12022, 12023, 12028, 12034, 12035, 12039, 12040, 12042, 12044, 12047, 12048, 12049, 12050, 12051, 12052, 12053, 12054, 12055, 12056, 12058, 12059, 12062, 12064, 12067, 12069, 12070, 12071, 13001, 13006, 13009, 13012, 13020, 13022, 1001, 1002, 1002.1, 1006, 1017, 1133, 1134, 2022, 2211, 5003, 6609, 6614, 6621, 6628, 6630, 6631, 6633, 6637, 6640, 6641, 6642, 6650, 7703, 8806, 10906, 10910, 10920, 10923, 10924, 10932, 10940, 10942, 10953, 11001, 12010, 12012, 12020, 12024, 12026, 12027, 12031, 13003, 13004, 13005, 13007, 13010, 13013, 13014, and 13015, and pharmaceutically acceptable salts and/or solvates thereof. Even more preferably, the compound of formula (I) is a compound selected from example numbers: 12036, 12038, 12041, 12057, 12060, 12061, 12065, 12068, 2020, 2212, 6601, 6602, 6617, 6618, 6622, 6624, 6626, 6629, 6639, 10901, 10904, 10925, 10926, 10928, 10930, 10931, 10964, 10972, 12001, 12007, 12008, 12009, 12014, 12015, 12016, 12017, 12018, 12019, 12021, 12022, 12023, 12028, 12034, 12035, 12039, 12040, 12042, 12044, 12047, 12048, 12049, 12050, 12051, 12052, 12053, 12054, 12055, 12056, 12058, 12059, 12062, 12064, 12067, 12069, 12070, 12071, 13001, 13006, 13009, 13012, 13020, and 13022, and pharmaceutically acceptable salts and/or solvates thereof. Yet more preferably, the compound of formula (I) is a compound selected from example numbers: 12036, 12038, 12041, 12057, 12060, 12061, 12065, and 12068, and pharmaceutically acceptable salts and/or solvates thereof. Yet more preferably still, the compound of formula (I) is a compound selected from example numbers 1001, 1002.1, 2020, 2022, 6602, 6624, 10901, 10906, 12001, and 12016, and pharmaceutically acceptable salts and/or solvates thereof. More preferably still, the compound of formula (I) is a compound selected from example numbers 1001, 1002.1, 2020, 2022, 6602, 6624, 10901, 10906, and 12001, and pharmaceutically acceptable salts and/or solvates thereof.

Preferably, the compound of formula (I) is a compound selected from example numbers: 1001, 1002, 1002.1, 1002.2, 1004, 1005.1, 1006, 1009, 1011, 1012, 1013, 1014, 1015, 1016, 1017, 1018, 1019, 2020, 2022, 1101, 1105, 1109, 1110, 1113, 1118, 4401, 6601, 6602, 7702, and 10901, and pharmaceutically acceptable salts and/or solvates thereof. More preferably, the compound of formula (I) is a compound selected from example numbers: 1001, 1002, 1002.1, 1006, 1017, 2020, 2022, 6601, 6602, and 10901, and pharmaceutically acceptable salts and/or solvates thereof. Even more preferably, the compound of formula (I) is a compound selected from example numbers: 1001, 1002, and 1005.1, and pharmaceutically acceptable salts and/or solvates thereof. Yet more preferably, the compound of formula (I) is a compound selected from example numbers: 1001 and 1002, and pharmaceutically acceptable salts and/or solvates thereof. Yet more preferably still, the compound of formula (I) is a compound selected from example numbers 1001, 1002.1, and 2020, and pharmaceutically acceptable salts and/or solvates thereof.

Preferably, the compound of formula (I) is a compound selected from example numbers: 1001, 1002, 1002.1, 1002.2, 1004, 1005.1, 1012, 1013, 1014, 1015, 1016, 1017, 1018, 1019, 2020, and 2022, and pharmaceutically acceptable salts and/or solvates thereof. More preferably, the compound of formula (I) is a compound selected from example numbers: 1001, 1002, 1002.1, 1017, 2020, and 2022, and pharmaceutically acceptable salts and/or solvates thereof. Even more preferably, the compound of formula (I) is a compound selected from example numbers: 1001, 1002, and 1005.1, and pharmaceutically acceptable salts and/or solvates thereof. Yet more preferably, the compound of formula (I) is a compound selected from example numbers: 1001 and 1002, and pharmaceutically acceptable salts and/or solvates thereof.

Preferably, the compound of formula (I) is a compound selected from example numbers: 1001, 1002, and 1005.1, and pharmaceutically acceptable salts and/or solvates thereof. More preferably, the compound of formula (I) is a compound selected from example numbers: 1001 and 1002, and pharmaceutically acceptable salts and/or solvates thereof.

### Therapeutic Applications

As noted above, the compounds (or pharmaceutically acceptable salts and/or solvates thereof), and pharmaceutical compositions comprising the compounds (or pharmaceutically acceptable salts and/or solvates thereof) of the present invention are inhibitors of FXIIa. They are therefore useful in the treatment of disease conditions for which FXIIa is a causative factor.

Accordingly, the present invention provides a compound of the invention (or a pharmaceutically acceptable salt and/or solvate thereof), or a pharmaceutical composition comprising a compound of the invention (or a pharmaceutically acceptable salt and/or solvate thereof), for use in medicine.

The present invention also provides for the use of a compound of the invention (or a pharmaceutically acceptable salt and/or solvate thereof), or a pharmaceutical composition comprising the compound of the invention (or a pharmaceutically acceptable salt and/or solvate thereof), in the manufacture of a medicament for the treatment or prevention of a disease or condition in which FXIIa activity is implicated.

The present invention also provides a method of treatment of a disease or condition in which FXIIa activity is implicated comprising administration to a subject in need thereof a therapeutically effective amount of a compound of the invention (or a pharmaceutically acceptable salt and/or solvate thereof), or a pharmaceutical composition comprising the compound of the invention (or a pharmaceutically acceptable salt and/or solvate thereof).

As discussed above, FXIIa can mediate the conversion of plasma kallikrein from plasma prekallikrein. Plasma kallikrein can then cause the cleavage of high molecular weight kininogen to generate bradykinin, which is a potent inflammatory hormone. Inhibiting FXIIa has the potential to inhibit (or even prevent) plasma kallikrein production. Thus, the disease or condition in which FXIIa activity is implicated can be a bradykinin-mediated angioedema.

The bradykinin-mediated angioedema can be non-hereditary. For example, the non-hereditary bradykinin-mediated angioedema can be selected from non-hereditary angioedema with normal C1 Inhibitor (AE-nC1 Inh), which can be environmental, hormonal, or drug-induced; acquired angioedema; anaphylaxis associated angioedema; angiotensin converting enzyme (ACE or ace) inhibitor-induced angioedema; dipeptidyl peptidase-4 inhibitor-induced angioedema; and tPA-induced angioedema (tissue plasminogen activator-induced angioedema).

Alternatively, and preferably, the bradykinin-mediated angioedema can be hereditary angioedema (HAE), which is angioedema caused by an inherited dysfunction/fault/mutation. Types of HAE that can be treated with compounds according to the invention include HAE type 1, HAE type 2, and normal C1 inhibitor HAE (normal C1 Inh HAE).

The disease or condition in which FXIIa activity is implicated can be selected from vascular hyperpermeability, stroke including ischemic stroke and haemorrhagic accidents; retinal edema; diabetic retinopathy; impaired visual acuity; DME; retinal vein occlusion; and AMD. These conditions can also be bradykinin-mediated.

As discussed above, FXIIa can activate FXIa to cause a coagulation cascade. Thrombotic disorders are linked to this cascade. Thus, the disease or condition in which FXIIa activity is implicated can be a thrombotic disorder. More specifically, the thrombotic disorder can be thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; acute respiratory distress syndrome (ARDS); idiopathic pulmonary fibrosis (IPF); rheumatoid arthritis (RA); and cold-induced urticarial autoinflammatory syndrome.

Surfaces of medical devices that come into contact with blood can cause thrombosis. The compounds (or pharmaceutically acceptable salts and/or solvates thereof) and pharmaceutical compositions of the present invention can be coated on the surfaces of devices that come into contact with blood to mitigate the risk of the device causing thrombosis. For instance, they can lower the propensity these devices to clot blood and therefore cause thrombosis. Examples of devices that come into contact with blood include vascular grafts, stents, in dwelling catheters, external catheters, orthopedic prosthesis, cardiac prosthesis, and extracorporeal circulation systems.

Other disease conditions for which FXIIa is a causative factor include: neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

Brown adipose tissue (BAT) thermogenic activity can be mediated by the kallikrein-kinin system, and impaired BAT activity is associated with obesity and insulin resistance. Inhibiting FXIIa has the potential to inhibit (or even prevent) BAT activity mediated by the kallikrein-kinin system. The compounds or pharmaceutically acceptable salts and/or solvates thereof) and pharmaceutical compositions of the invention can therefore treat disease conditions such as obesity and diabetes.

### Combination Therapy

The compounds of the present invention (or pharmaceutically acceptable salts and/or solvates thereof) may be administered in combination with other therapeutic agents. Suitable combination therapies include any compound of the present invention (or a pharmaceutically acceptable salt and/or solvate thereof) combined with one or more agents selected from agents that inhibit platelet-derived growth factor (PDGF), endothelial growth factor (VEGF), integrin alpha5beta1, steroids, other agents that inhibit FXIIa and other inhibitors of inflammation.

Some specific examples of therapeutic agents that may be combined with the compounds of the present invention include those disclosed in EP2281885A1 and by S. Patel in Retina, 2009 June; 29(6 Suppl): S45-8.

Other suitable combination therapies include a compound of the invention (or a pharmaceutically acceptable salt and/or solvate thereof) combined with one or more agents selected from agents that treat HAE (as defined generally herein), for example bradykinin B2 antagonists such icatibant (Firazyr®); plasma kallikrein inhibitors such as ecallantide (Kalbitor®), lanadelumab (Takhzyro®) and berotralstat (ORLADEYO™); or C1 esterase inhibitor such as Cinryze® and Haegarda® and Berinert® and Ruconest®.

Other suitable combination therapies include a compound of the invention (or a pharmaceutically acceptable salt and/or solvate thereof) combined with one or more agents selected from agents that are antithrombotics (as outlined above), for example other Factor XIIa inhibitors, thrombin receptor antagonists, thrombin inhibitors, factor VIIa inhibitors, factor Xa inhibitors, factor XIa inhibitors, factor IXa inhibitors, adenosine diphosphate antiplatelet agents (e.g., P2Y12 antagonists), fibrinogen receptor antagonists (e.g. to treat or prevent unstable angina or to prevent reocclusion after angioplasty and restenosis) and aspirin) and platelet aggregation inhibitors.

When combination therapy is employed, the compounds of the present invention and said combination agents may exist in the same or different pharmaceutical compositions, and may be administered separately, sequentially or simultaneously.

The compounds of the present invention can be administered in combination with laser treatment of the retina. The combination of laser therapy with intravitreal injection of an inhibitor of VEGF for the treatment of diabetic macular edema is known (Elman M, Aiello L, Beck R, et al. “Randomized trial evaluating ranibizumab plus prompt or deferred laser or triamcinolone plus prompt laser for diabetic macular edema” Ophthalmology. 27 Apr. 2010).

### Definitions

As noted above, the term “alkyl” is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from (C₁-C₆)alkoxy, OH, —NR13R14, —C(═O)OR13, —C(═O)NR13R14, CN, CF₃, halo. As noted above “alkyl^{b}” is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from (C₁-C₆)alkoxy, OH, CN, CF₃, halo. Examples of such alkyl or alkyl^{b} groups include, but are not limited, to C1-methyl, C2-ethyl, C3-propyl and C4-n-butyl, C3-iso-propyl, C4-sec-butyl, C4-iso-butyl, C4-tert-butyl and C5-neo-pentyl, optionally substituted as noted above. More specifically, “alkyl” or “alkyl^{b}” can be a linear saturated hydrocarbon having up to 6 carbon atoms (C₁-C₆) or a branched saturated hydrocarbon of between 3 and 6 carbon atoms (C₃-C₆), optionally substituted as noted above. Even more specifically, “alkyl” or “alkyl^{b}” can be a linear saturated hydrocarbon having up to 4 carbon atoms (C₁-C₄) or a branched saturated hydrocarbon of between 3 and 4 carbon atoms (C₃-C₄), optionally substituted as noted above, which is herein called “small alkyl” or “small alkyl^{b}”, respectively. Preferably, “alkyl” or “alkyl^{b}” can be defined as a “small alkyl” or “small alkyl^{b}”.

“Aryl” and “aryl^{b}” are as defined above. Typically, “aryl” or “aryl^{b}” will be optionally substituted with 1, 2 or 3 substituents. Optional substituents are selected from those stated above. Examples of suitable aryl or aryl^{b} groups include phenyl, biphenyl and naphthyl (each optionally substituted as stated above).

Preferably “aryl” is selected from phenyl, substituted phenyl (wherein said substituents are selected from those stated above) and naphthyl. Most preferably “aryl” is selected from phenyl and substituted phenyl (wherein said substituents are selected from those stated above).

As noted above, the term “cycloalkyl” is a monocyclic saturated hydrocarbon ring of between 3 and 6 carbon atoms (C₃-C₆); cycloalkyl may optionally be substituted with 1 or 2 substituents independently selected from alkyl, (C₁-C₆)alkoxy, OH, CN, CF₃, halo. Examples of suitable monocyclic cycloalkyl groups include cyclopropyl, cyclobutyl, cyclopentyl, and cyclohexyl, optionally substituted as noted above. More specifically, “cycloalkyl” can be a monocyclic saturated hydrocarbon ring of between 3 and 5 carbon atoms, more specifically, between 3 and 4 carbon atoms, optionally substituted as noted above.

As noted above, the term “alkoxy” is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, and fluoro. Examples of such alkoxy groups include, but are not limited to, C1-methoxy, C2-ethoxy, C3-n-propoxy and C4-n-butoxy for linear alkoxy, and C3-iso-propoxy, and C4-sec-butoxy and tert-butoxy for branched alkoxy, optionally substituted as noted aboves. More specifically, “alkoxy” can be linear groups of between 1 and 4 carbon atoms (C₁-C₄), more specifically, between 1 and 3 carbon atoms (C₁-C₃). More specifically, “alkoxy” can be branched groups of between 3 and 4 carbon atoms (C₃-C₄), optionally substituted as noted above.

“Halo” can be selected from Cl, F, Br and I. More specifically, halo can be selected from C1 and F.

As noted above, “heteroaryl” is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃. For example, heteroaryl can be selected from thiophene, furan, pyrrole, pyrazole, imidazole, oxazole, isoxazole, thiazole, isothiazole, triazole, oxadiazole, thiadiazole, pyridine, pyridazine, pyrimidine, and pyrazine, optionally substituted as noted above.

“Heteroaryl^{a}” and “heteroaryl^{b}” are as defined above. Typically, “heteroaryl^{a}” or “heteroaryl^{b}” will be optionally substituted with 1, 2 or 3 substituents. Optional substituents are selected from those stated above. Examples of suitable heteroaryl^{a} or heteroaryl^{b} groups include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, indolyl, benzimidazolyl, benzotriazolyl, quinolinyl, isoquinolinyl, 5-azathianaphthenyl, indolizinyl, isoindolyl, indazolyl, benzothiazolyl, cinnolinyl, quinazolinyl, quinoxalinyl, 1,8-napthyridinyl and phthalazinyl (optionally substituted as stated above). More specifically, “heteroaryl^{a}” or “heteroaryl^{b}” can be a 9- or 10-membered bi-cyclic ring as defined, and optionally substituted as stated above. Examples of suitable 9- or 10-membered heteroaryl^{a} or heteroaryl^{b} groups include indolyl, benzimidazolyl, benzotriazolyl, quinolinyl, isoquinolinyl, 5-azathianaphthenyl, indolizinyl, isoindolyl, indazolyl, benzothiazolyl, cinnolinyl, quinazolinyl, quinoxalinyl, 1,8-napthyridinyl and phthalazinyl.

As noted above, “heterocycloalkyl” is a non-aromatic carbon-containing monocyclic ring containing 3, 4, 5, or 6, ring members, wherein at least one ring member is independently selected from N, NR12, S, and O; heterocycloalkyl may be optionally be substituted with 1 or 2 substituents independently selected from alkyl, (C₁-C₆)alkoxy, OH, CN, CF₃, halo. More specifically, “heterocycloalkyl” can be a non-aromatic carbon-containing monocyclic ring containing 3, 4, 5, or 6, ring members, wherein at least one ring member is independently selected from NR12, and O; heterocycloalkyl may be optionally substituted with 1 or 2 substituents independently selected from alkyl (C₁-C₅)alkoxy, OH, CN, CF₃, halo.

The term “O-linked”, such as in “O-linked hydrocarbon residue”, means that the hydrocarbon residue is joined to the remainder of the molecule via an oxygen atom.

In groups such as —(CH₂)₀₋₆-A, “-” denotes the point of attachment of the substituent group to the remainder of the molecule.

As is clear from the definitions above, and for the avoidance of any doubt, it will be understood that “Y” is defined above, and does not encompass Yttrium.

As is clear from the definitions above, and for the avoidance of any doubt, it will be understood that “B” is defined above, and does not encompass Boron.

As is clear from the definitions above, and for the avoidance of any doubt, it will be understood that “W” is defined above, and does not encompass Tungsten.

As is clear from the definitions above, and for the avoidance of any doubt, it will be understood that “V” is defined above, and does not encompass Vanadium.

As is clear from the definitions above, and for the avoidance of any doubt, it will be understood that “U” is defined above, and does not encompass Uranium.

“Salt”, as used herein (including “pharmaceutically acceptable salt”) means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, base addition salts (including pharmaceutically acceptable base addition salts) that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, acid addition salts (including pharmaceutically acceptable acid addition salts) that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates, trifluoroacetates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming prodrugs are described in ‘The Practice of Medicinal Chemistry, 2^{nd} Ed. pp 561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Where compounds of the invention exist in one or more geometric, optical, enantiomeric, diastereomeric and tautomeric forms, including but not limited to cis- and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis). For example, where compounds of the invention exist as a mixture of stereoisomers, one stereoisomer can be present at a purity of >90% relative to the remaining stereoisomers, or more specifically at a purity of >95% relative to the remaining stereoisomers, or yet more specifically at a purity of >99% relative to the remaining stereoisomers. For example, where compounds of the invention exists in enantiomeric forms, the compound can be >90% enantiomeric excess (ee), or more specifically >95% enantiomeric excess (ee), or yet more specifically, >99% ee.

Unless otherwise stated, the compounds of the invention include compounds that differ only in the presence of one or more isotopically enriched atoms. For example, compounds wherein hydrogen is replaced by deuterium or tritium, or wherein carbon is replaced by ¹³C or ¹⁴C, are within the scope of the present invention. Such compounds are useful, for example, as analytical tools or probes in biological assays.

Methods for the preparation of deuterated isotopes will be readily apparent to those skilled in the art. For example, methods may include the use of deuterated starting materials in the synthesis of the compounds described herein. Deuterated starting materials will be readily available to the skilled person, from standard commercial sources. Methods for making deuterated isotopes and deuterated starting materials may also include deuterium exchange. For example, deuterium exchange may be achieved by mixing the compounds with D₂O.

In the context of the present invention, references herein to “treatment” include references to curative, palliative and prophylactic treatment. For instance, treatment includes preventing the symptoms of the disease conditions for which FXIIa is a causative factor.

### Methods

The compounds of the invention may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Compounds of the invention intended for pharmaceutical use may be administered as a solid or liquid, such as a tablet, capsule or solution. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly, the present invention provides a pharmaceutical composition comprising a compound of the invention and a pharmaceutically acceptable carrier, diluent or excipient.

For the treatment of conditions such as retinal vascular permeability associated with diabetic retinopathy and diabetic macular edema, the compounds of the invention may be administered in a form suitable for injection into the ocular region of a patient, in particular, in a form suitable for intra-vitreal injection. It is envisaged that formulations suitable for such use will take the form of sterile solutions of a compound of the invention in a suitable aqueous vehicle. The compositions may be administered to the patient under the supervision of the attending physician.

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, excipients such as sugars (including but not restricted to glucose, manitol, sorbitol, etc.), salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of co-solvents and/or solubility-enhancing agents such as surfactants, micelle structures and cyclodextrins.

Preferably, the compounds of the invention are administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid plugs, solid microparticulates, semi-solids and liquids (including multiple phases or dispersed systems). Exemplary formulations suitable for oral administration include tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Liquid (including multiple phases and dispersed systems) formulations include emulsions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, New York, 1980).

For administration to human patients, the total daily dose of the compounds of the invention is typically in the range 0.1 mg and 10,000 mg, or between 1 mg and 5000 mg, or between 10 mg and 1000 mg depending, of course, on the mode of administration.

The total dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

### NUMBERED EMBODIMENTS

The invention is also described by the following numbered embodiments:

1. A compound of formula (I),

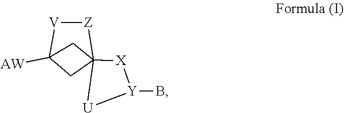

wherein:
U is absent, —C(R16)(R17)-, CH₂C(R16)(R17) or C(R16)(R17)CH₂;
-V-Z- is:
   absent, —CH₂—, or —CH₂—O—CH₂; or
V is selected from CH₂, O and NR18, and Z is selected from —C(R16)(R17)-CH₂— and —C(R16)(R17)-; or,
V is selected from —CH₂—C(R16)(R17)- and —C(R16)(R17)-, and Z is selected from CH₂, O and NR18;
   wherein R18 is selected from H, alkyl, (CH₂)₀₋₆-aryl, (CH₂)₀₋₆-heteroaryl^{a}, C(═O)R19, C(═O)OR19, C(═O)NHR19, C(═O)N(alkyl)(R19), C(═O)SR19 and SO₂R19;
   wherein R19 is selected from alkyl, cycloalkyl, (CH₂)₀₋₆-aryl and (CH₂)₀₋₆-heteroaryl^{a};
X is selected from a bond, O, CR1R2, C═O and NR12;
Y is, where possible, selected from O, CR1R2, CR1, C═O, N and NR12;
   R1 is selected from H, alkyl, alkoxy, OH, halo and NR13R14;
   R2 is selected from H and small alkyl;
wherein when one of X or Y is C═O, the other is, where possible, O, CR1R2, CR1, N or NR12;
wherein when X is NR12, Y is, where possible, CR1R2, CR1 or C═O;
wherein when Y is, where possible, NR12 or N, X is a bond, CR1R2 or C═O;
wherein when X is O, Y is, where possible, CR1R2, CR1 or C═O;
wherein when Y, where possible, is O, X is a bond, CR1R2 or C═O;
wherein when X is a bond, Y is, where possible, O, N or NR12;
wherein when U is not absent, -V-Z- is absent;
wherein when -V-Z- is not absent, U is absent;
B is selected from:
   (i) heteroaryl^{a};
   (ii) aryl;
   (iii) a 5- to 6-membered non-aromatic heterocyclic ring containing one N ring member, which is unsaturated with 1 or 2 double bonds, wherein the non-aromatic heterocyclic ring is optionally substituted by 1, 2 or 3 substituents independently selected from alkyl, alkoxy, aryl^{b}, OH, OCF₃, halo, oxo, CN, and CF₃; and
   (iv) a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring may be optionally substituted with 1, 2, or 3 substituted by up to three substituents independently selected from alkyl, alkoxy, OH, OCF₃, halo, oxo, CN, and CF₃, wherein the 6,5-bicyclic ring may be attached via the 6- or 5-membered ring;
AW- is selected from:
—(CH₂)₀₋₆—(CHR15)-(CH₂)₀₋₆-A, —(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—NR12-(CH₂)₁₋₆—C(═O)-A, —(CH₂)₀₋₆—NH—C(═O)—(CH₂)₀₋₆-A, —C(═O)NR12-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A and —SO₂—NH-A;

A is a 4- to 15-membered mono-, bi-, or tri-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein when A is a bicyclic ring system, the bicyclic ring system is fused, bridged or spiro;
wherein when A is a tricyclic ring system, each of the three rings in the tricyclic ring system is either fused, bridged or spiro to at least one of the other rings in the tricyclic ring system;
wherein when -V-Z- is —CH₂—, U is absent, and AW- is A-(C═O)—, A may not be substituted by —(CH₂)₀-heteroaryl;
alkyl is a linear saturated hydrocarbon having up to 10 carbon atoms (C₁-C₁₀) or a branched saturated hydrocarbon of between 3 and 10 carbon atoms (C₃-C₁₀); alkyl may optionally be substituted with 1, 2 or 3 substituents independently selected from (C₁-C₆)alkoxy, OH, —NR13R14, —C(═O)OR13, —C(═O)NR13R14, CN, CF₃, halo;
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl^{b} may optionally be substituted with 1, 2 or 3 substituents independently selected from (C₁-C₅)alkoxy, OH, CN, CF₃, halo;
small alkyl is a linear saturated hydrocarbon having up to 4 carbon atoms (C₁-C₄) or a branched saturated hydrocarbon of between 3 and 4 carbon atoms (C₃-C₄); small alkyl may optionally be substituted with 1 or 2 substituents independently selected from (C₁-C₆)alkoxy, OH, NR13R14, C(═O)OR13, C(═O)NR13R14, CN, CF₃, halo;
aryl is phenyl, biphenyl or naphthyl; aryl may be optionally substituted with 1, 2 or 3 substituents independently selected from alkyl, alkoxy, methylenedioxy, ethylenedioxy, OH, halo, CN, —(CH₂)₀₋₃—O-heteroaryl^{a}, aryl^{b}, —O-aryl^{b}, —(CH₂)₁₋₃-aryl^{b}, —(CH₂)₀₋₃-heteroaryl^{a}, —C(═O)OR13, —C(═O)NR13R14, —(CH₂)₀₋₃—NR13R14, OCF₃ and CF₃;
aryl^{b} is phenyl, biphenyl or naphthyl; aryl^{b} may be optionally substituted with 1, 2 or 3 substituents independently selected from alkyl^{b}, alkoxy, OH, halo, CN, and CF₃;
cycloalkyl is a monocyclic saturated hydrocarbon ring of between 3 and 6 carbon atoms (C₃-C₆);
cycloalkyl may optionally be substituted with 1 or 2 substituents independently selected from alkyl, (C₁-C₆)alkoxy, OH, CN, CF₃, halo;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1 or 2 substituents independently selected from OH, CN, CF₃, and fluoro;
halo is F, Cl, Br, or I;
heteroaryl is a 5- or 6-membered carbon-containing aromatic ring containing one, two or three ring members that are selected from N, NR8, S, and O; heteroaryl may be optionally substituted with 1, 2 or 3 substituents independently selected from alkyl, alkoxy, OH, OCF₃, halo, CN, and CF₃;
heteroaryl^{a} is a 5, 6, 9 or 10 membered mono- or bi-cyclic aromatic ring, containing, where possible, 1, 2, 3 or 4 ring members independently selected from N, NR12, S and O; heteroaryl^{a} may be optionally substituted with 1, 2 or 3 substituents independently selected from alkyl, alkoxy, OH, OCF₃, halo, CN, aryl^{b}, —(CH₂)₀₋₃—NR13R14, heteroaryl^{b}, —C(═O)OR12, —C(═O)NR13R14 and CF₃;
heteroaryl^{b} is a 5, 6, 9 or 10 membered mono- or bi-cyclic aromatic ring, containing, where possible, 1, 2 or 3 ring members independently selected from N, NR12, S and O; wherein heteroaryl^{b} may be optionally substituted with 1, 2 or 3 substituents independently selected from alkyl^{b}, alkoxy, OH, halo, CN, aryl^{b}, —(CH₂)₁₋₃-aryl^{b}, and CF₃;
R8 is independently selected from H, alkyl, cycloalkyl, and heterocycloalkyl;
heterocycloalkyl is a non-aromatic carbon-containing monocyclic ring containing 3, 4, 5, or 6, ring members, wherein at least one ring member is independently selected from N, NR12, S, and O;
heterocycloalkyl may be optionally be substituted with 1 or 2 substituents independently selected from alkyl, (C₁-C₆)alkoxy, OH, CN, CF₃, halo;
R12 is independently selected from H, alkyl, and cycloalkyl;
R13 and R14 are independently selected from H, alkyl^{b}, aryl^{b} and heteroaryl^{b} or R13 and R14 together with the nitrogen atom to which they are attached form a carbon-containing 4-, 5-, 6- or 7-membered heterocyclic ring, optionally containing an additional heteroatom selected from N, NR12, S, SO, SO₂, and O, which may be saturated or unsaturated with 1 or 2 double bonds and which may be optionally mono- or di-substituted with substituents selected from oxo, alkyl^{b}, alkoxy, OH, halo and CF₃;
R15 is selected from alkyl, halo, CF₃, CN, OH, alkoxy, NR13R14, and CONR13R14;
R16 and R17 are independently selected from H and small alkyl;
and tautomers, isomers, stereoisomers (including enantiomers, diastereoisomers and racemic and scalemic mixtures thereof), deuterated isotopes, and pharmaceutically acceptable salts and/or solvates thereof;
wherein the compound is not N-(2-chlorophenyl)-3-((5-cyano-1H-indazol-1-yl)-methyl)-N-methylbicyclo-[1.1.1]pentane-1-carboxamide.

2. A compound of formula (I) according to numbered embodiment 1, or a tautomer, isomer, stereoisomer (including an enantiomer, a diastereoisomer and a racemic and scalemic mixture thereof), a deuterated isotope, and a pharmaceutically acceptable salt and/or solvate thereof, wherein R18 is selected from alkyl, (CH₂)₀₋₆-aryl, (CH₂)₀₋₆-heteroaryl^{a}, C(═O)R19, C(═O)OR19, C(═O)NHR19, C(═O)N(alkyl)(R19), C(═O)SR19 and SO₂R19.

3. A compound of formula (I) according to any of numbered embodiments 1 to 2, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein U is absent,
which is a compound of formula (Ia)

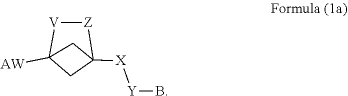

4. A compound of formula (I) according to any of numbered embodiments 1 to 2, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein -V-Z- is absent,
which is a compound of formula (Ib)

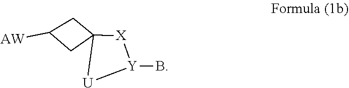

5. A compound of formula (I) according to any of numbered embodiments 1 to 4, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein when -V-Z- is absent and U is absent, and AW-and-XYB are trans to one another which is a compound of formula (Ic)

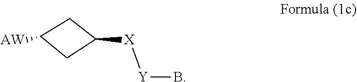

6. A compound of formula (I) according to any of numbered embodiments 1 to 3, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when not absent, -V-Z- is selected from:
   —CH₂—, or
V is selected from CH₂, O and NR18, and Z is selected from —C(R16)(R17)-CH₂— and —C(R16)(R17)-; or,
V is selected from —CH₂—C(R16)(R17)- and —C(R16)(R17)-, and Z is selected from CH₂, O and NR18; or wherein when -V-Z- is absent:
U is absent, CH₂ or —CH₂CH₂—.

7. A compound of formula (I) according to any of numbered embodiments 1 to 3, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂—, —CH₂—N(R18)- and —N(R18)-CH₂—; or
when -V-Z- is absent:
U is absent, CH₂ or —CH₂CH₂—.

8. A compound of formula (I) according to any of numbered embodiments 1 to 3, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂—; or
when -V-Z- is absent:
U is absent, CH₂ or —CH₂CH₂—.

9. A compound of formula (I) according to any of numbered embodiments 1 to 3, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂—, —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or
wherein when -V-Z- is absent:
U is absent, CH₂ or —CH₂CH₂—.

10. A compound of formula (I) according to any of numbered embodiments 1 to 3, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or
wherein when -V-Z- is absent:
U is absent, CH₂ or —CH₂CH₂—.

11. A compound of formula (I) according to any of numbered embodiments 1 to 3, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, —CH₂—CH₂—CH₂—, —CH₂—N(R18)- and —N(R18)-CH₂— wherein R18 is selected from:

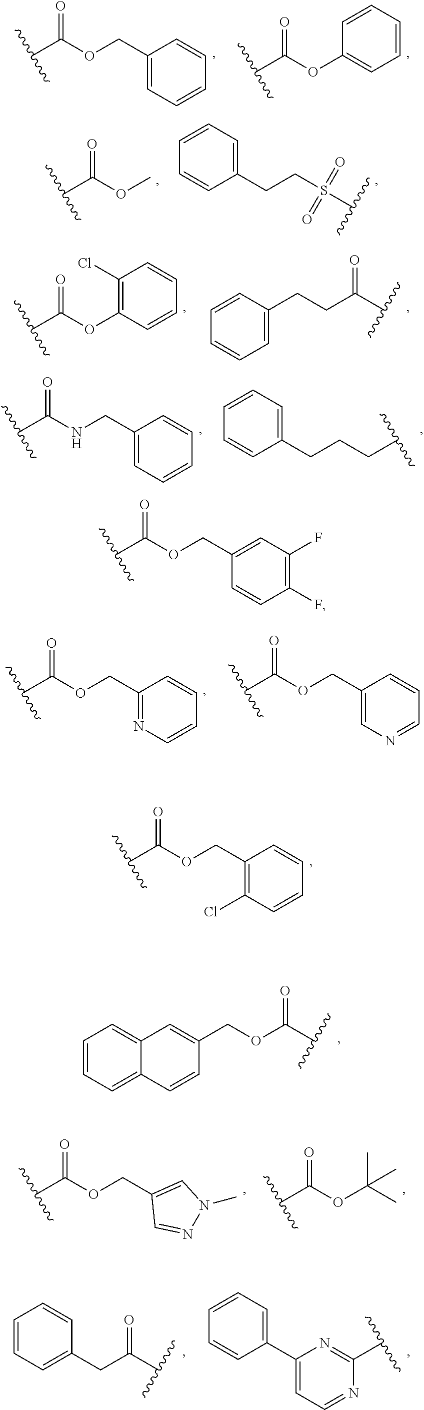

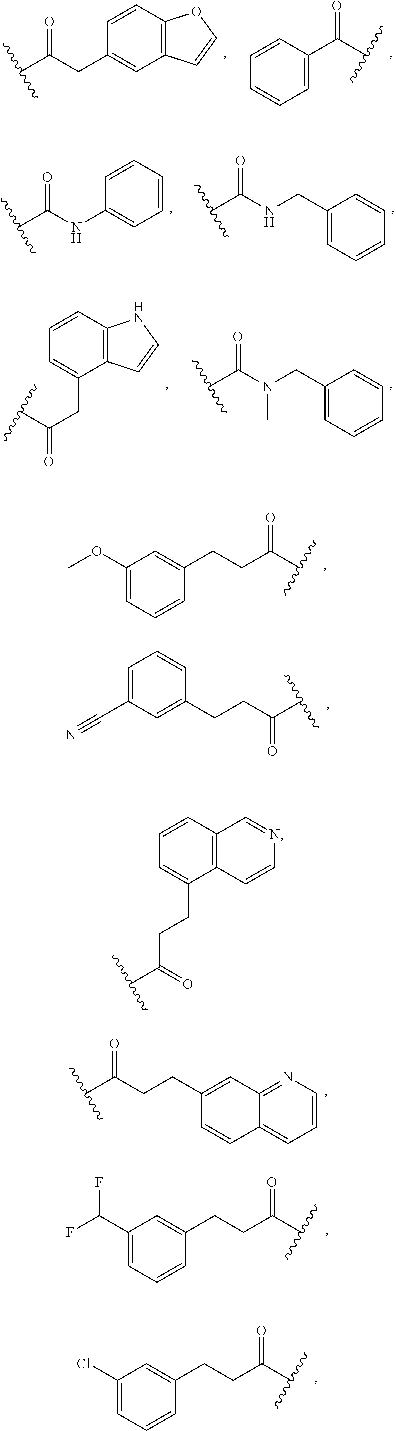

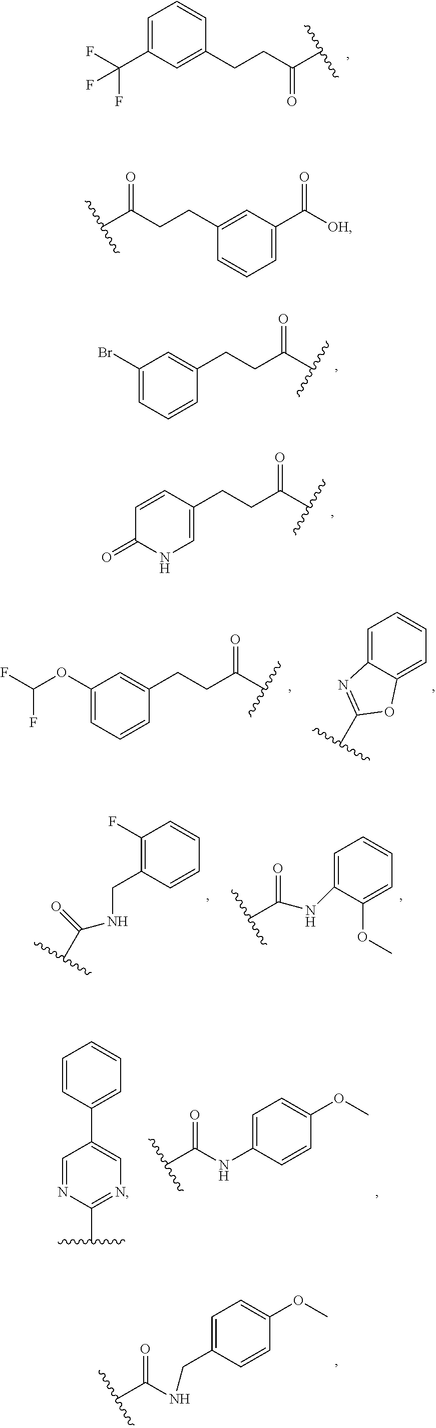

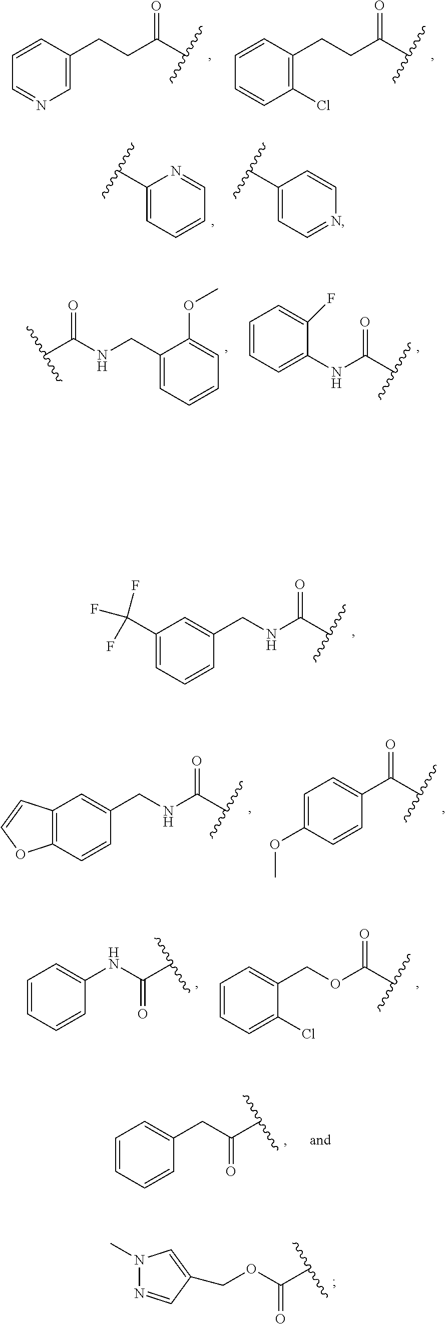

or
wherein when -V-Z- is absent:
U is absent, CH₂ or —CH₂CH₂—.

12. A compound of formula (I) according to any of numbered embodiments 1 to 3, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- and —N(R18)-CH₂— wherein R18 is selected from:

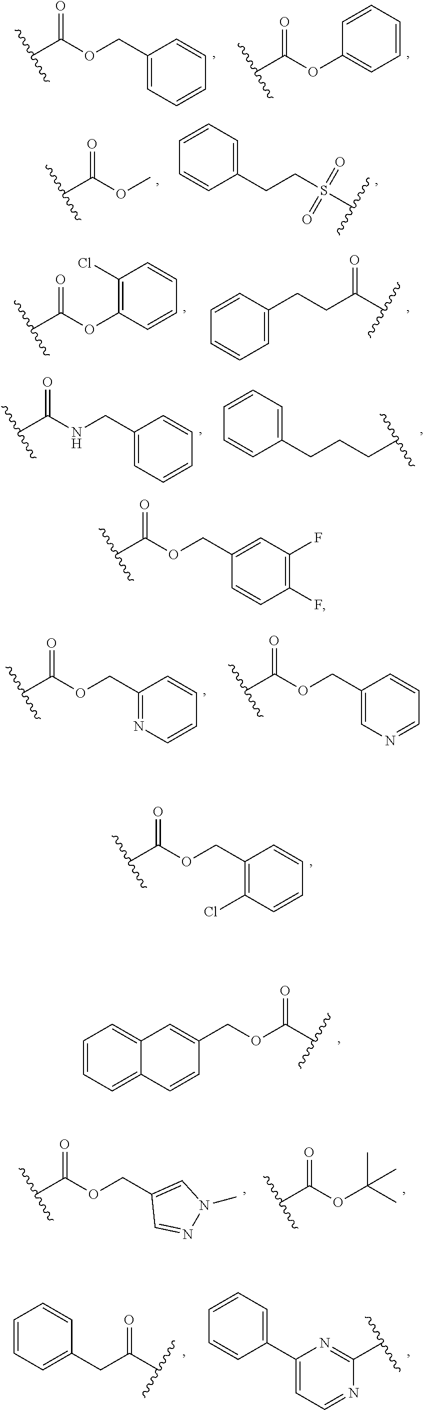

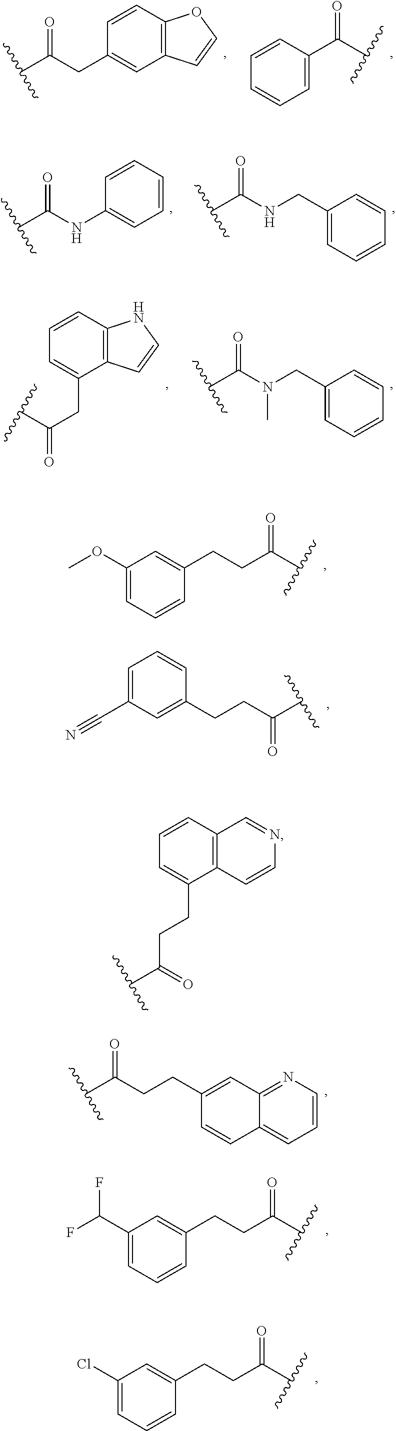

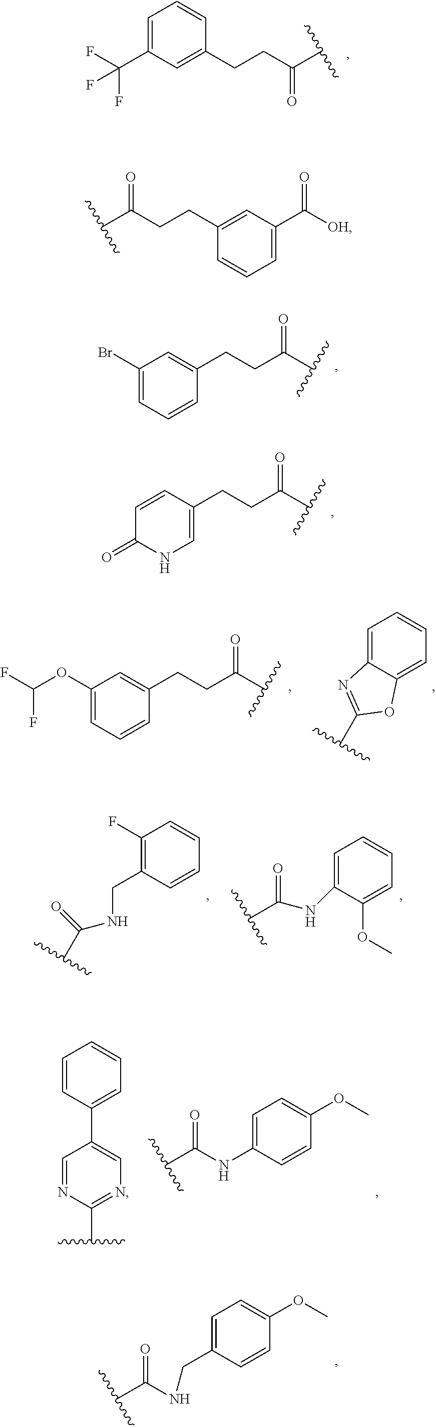

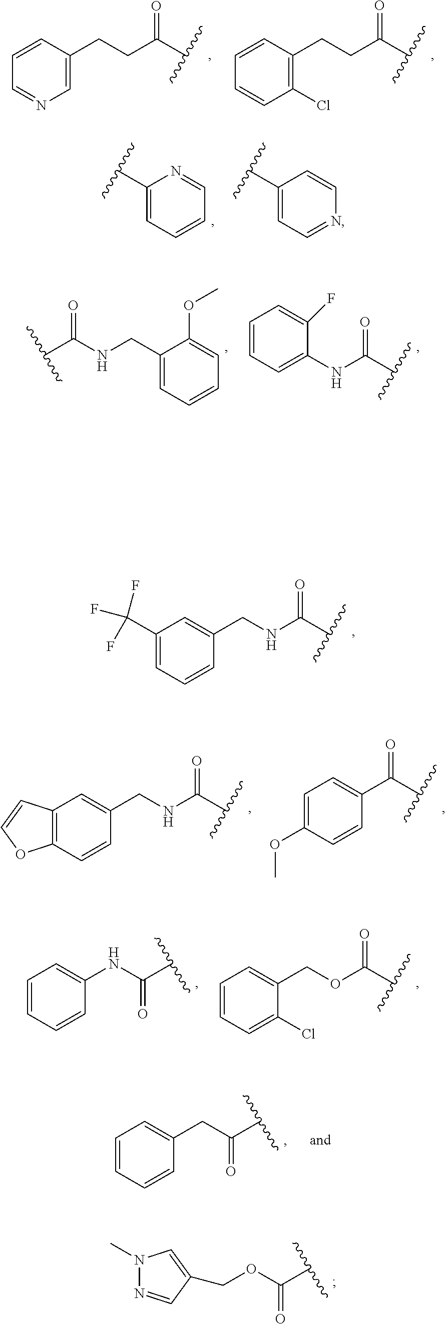

or
wherein when -V-Z- is absent:
U is absent, CH₂ or —CH₂CH₂—.

13. A compound of formula (I) according to any of numbered embodiments 1 to 2, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, and —CH₂—CH₂—CH₂—; or,
wherein when -V-Z- is absent:
U is absent, CH₂ or —CH₂CH₂—.

14. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof, wherein, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —CH₂—CH₂—O—, —CH₂—CH₂—CH₂—, —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or
when -V-Z- is absent:
U is absent, CH₂ or —CH₂CH₂—.

15. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof, wherein, when not absent, -V-Z- is selected from:
—CH₂—, —O—CH₂—, —O—C(CH₃)₂—, —CH₂—O—, —C(CH₃)₂—O—, —N(COCH₃)—CH₂, —CH₂—N(COCH₃), —CH₂—CH₂—, —CH₂—CH₂—O—, —CH₂—CH₂—CH₂—, —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or
when -V-Z- is absent:
U is absent, CH₂ or —CH₂CH₂—.

16. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof, wherein, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —CH₂—CH₂—O—, —CH₂—CH₂—CH₂—, —CH₂—N(R18)- and —N(R18)-CH₂— wherein R18 is selected from:

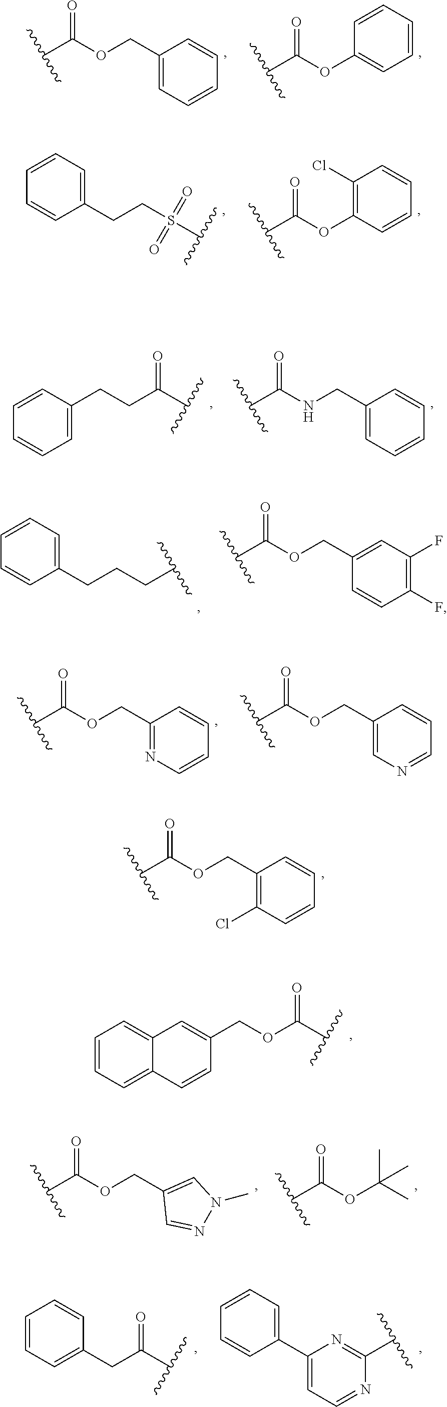

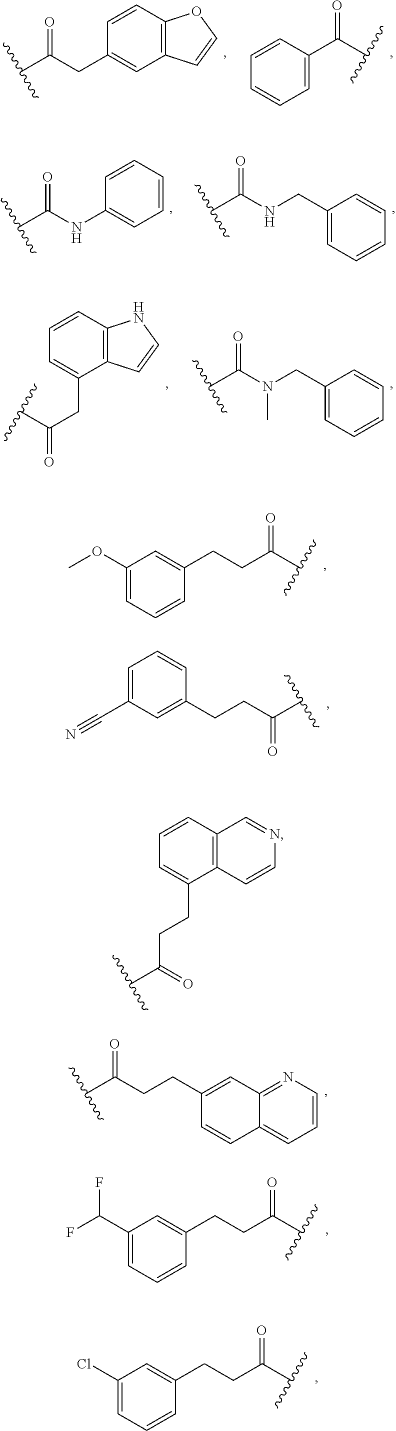

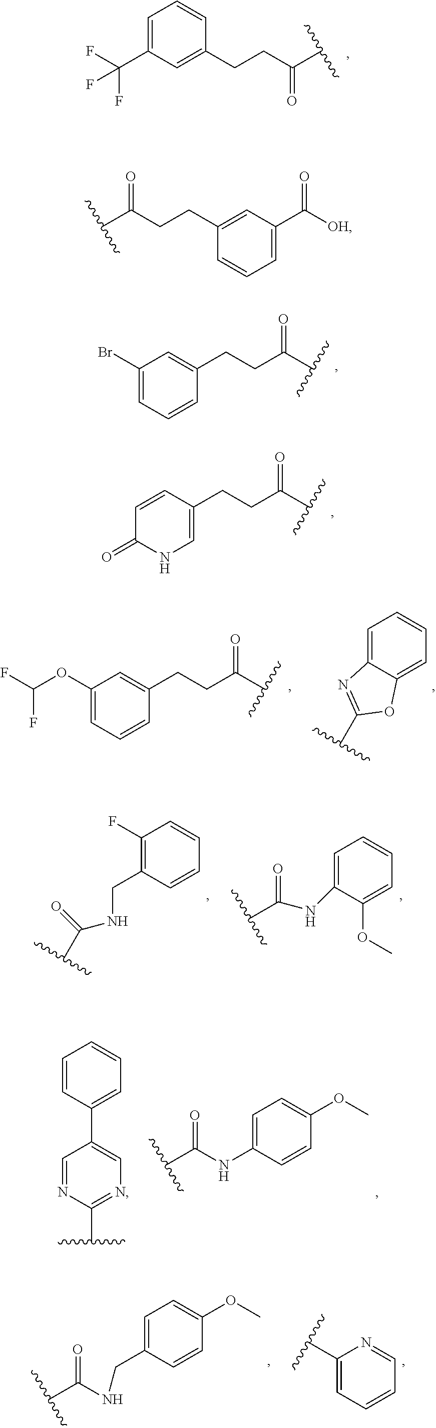

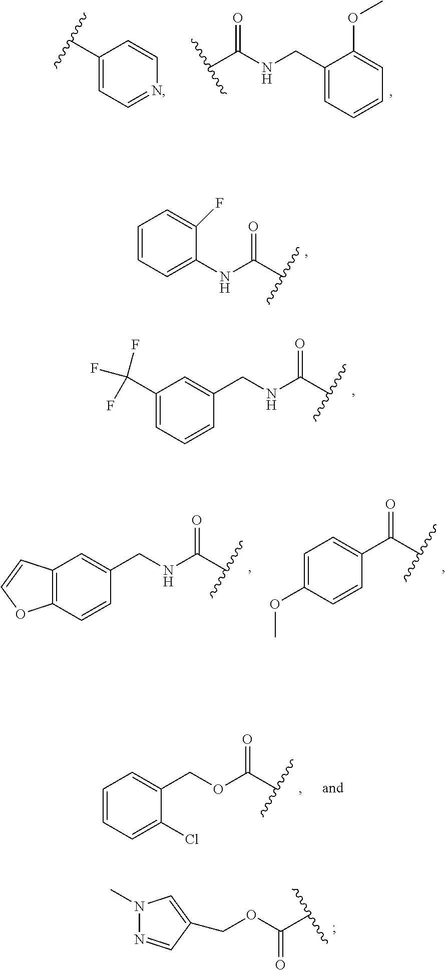

or
wherein when -V-Z- is absent:
U is absent, CH₂ or —CH₂CH₂—.

17. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof, wherein, when not absent, -V-Z- is selected from:
—CH₂—, —O—CH₂—, —O—C(CH₃)>, —CH₂—O—, —C(CH₃)>—O—, —N(COCH₃)—CH₂,
—CH₂—N(COCH₃), —CH₂—CH₂—, —CH₂—CH₂—O—, —CH₂—CH₂—CH₂—, —CH₂—N(R18)- and —N(R18)-CH₂— wherein R18 is selected from:

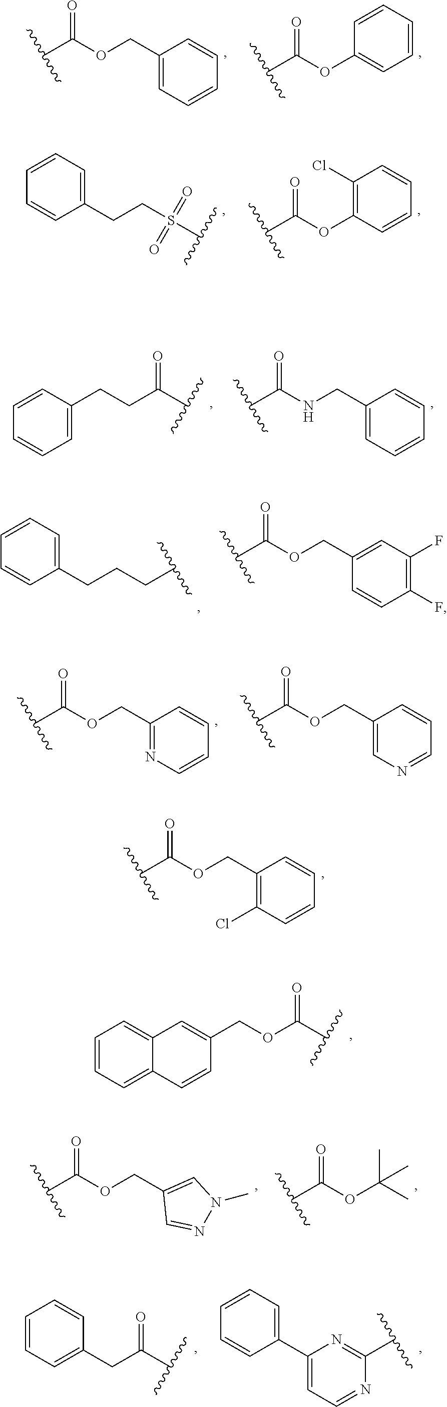

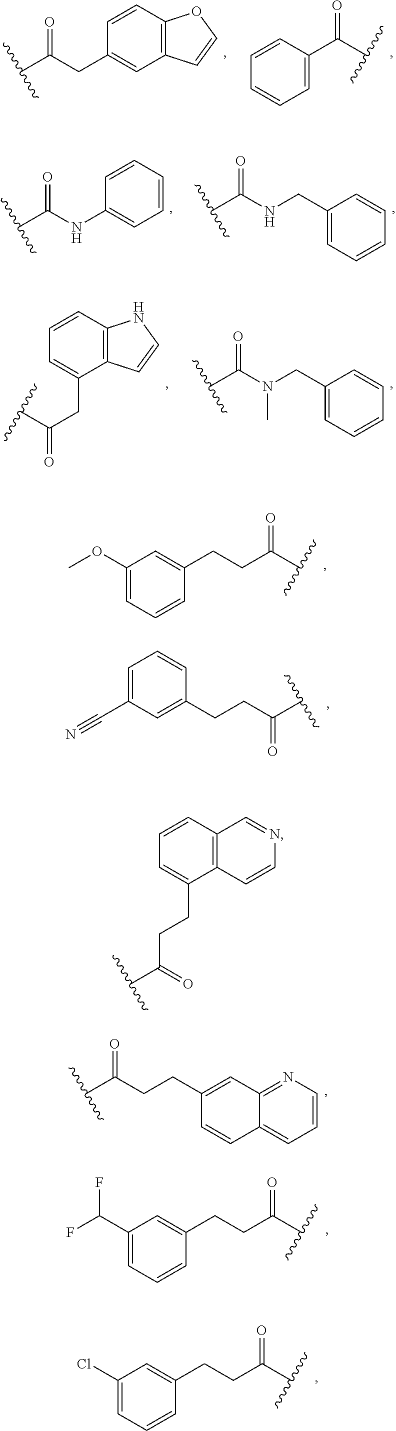

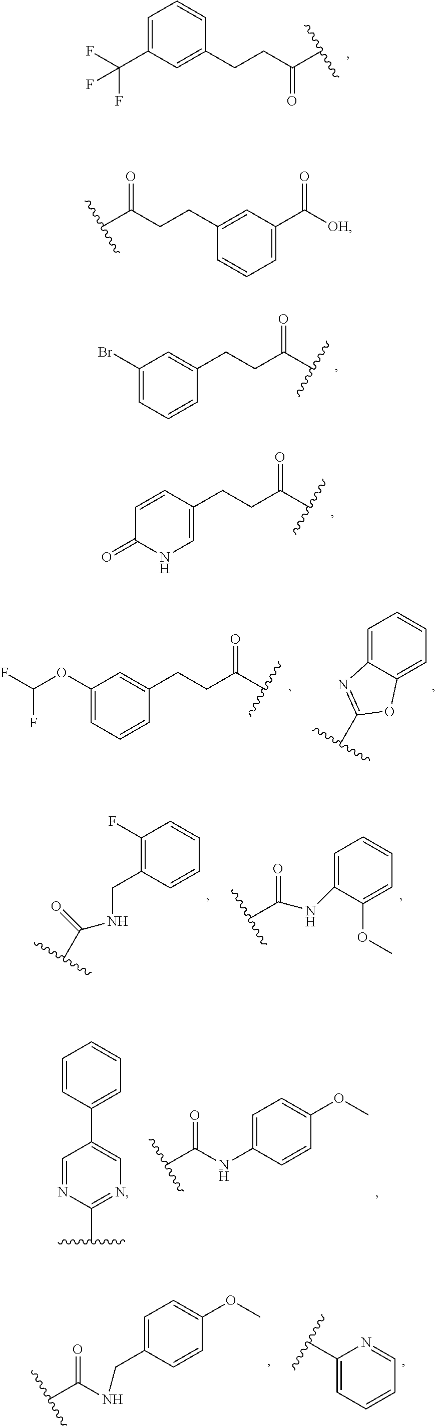

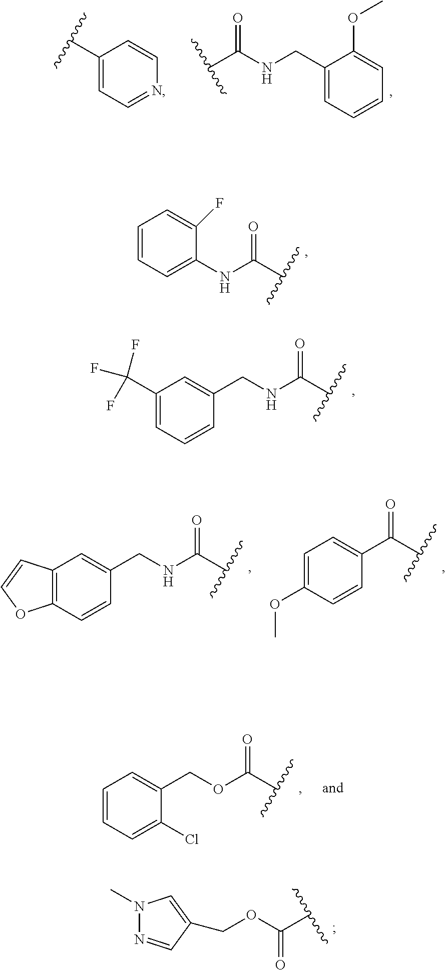

or
wherein when -V-Z- is absent:
U is absent, CH₂ or —CH₂CH₂—.

18. A compound of formula (I) according to numbered embodiment 13, or a tautomer, isomer, stereoisomer (including an enantiomer, a diastereoisomer and a racemic and scalemic mixture thereof), a deuterated isotope, and a pharmaceutically acceptable salt and/or solvate thereof, wherein, when not absent, -V-Z- is selected from:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —CH₂—CH₂—O—, and —CH₂—CH₂—CH₂—; or,
wherein when -V-Z- is absent:
U is absent, CH₂ or —CH₂CH₂—.

19. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof, wherein -V-Z- is selected from —CH₂—, —O—CH₂—, —CH₂—O—, —CH₂—CH₂—O—, —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
wherein R19 is selected from alkyl, (CH₂)₀₋₆-aryl and (CH₂)₀₋₆-heteroaryl^{a}; or
when -V-Z- is absent:
U is absent, CH₂ or —CH₂CH₂—.

20. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof, wherein -V-Z- is selected from —CH₂—, —O—CH₂—, —CH₂—O—, —CH₂—CH₂—O—, —CH₂—N(R18)- and —N(R18)-CH₂— wherein R18 is selected from:

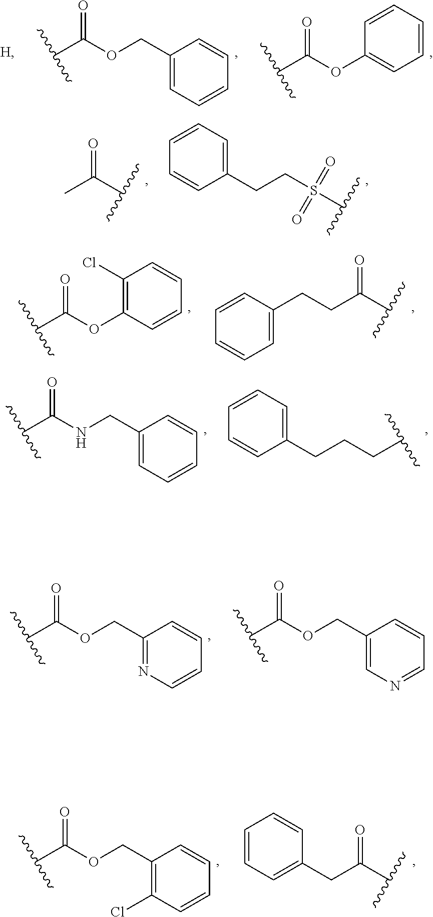

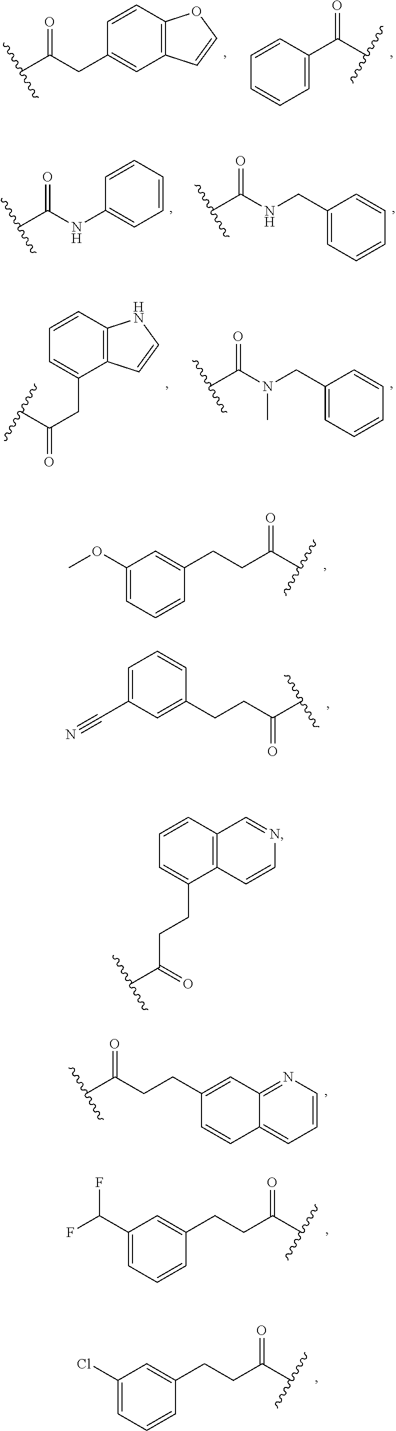

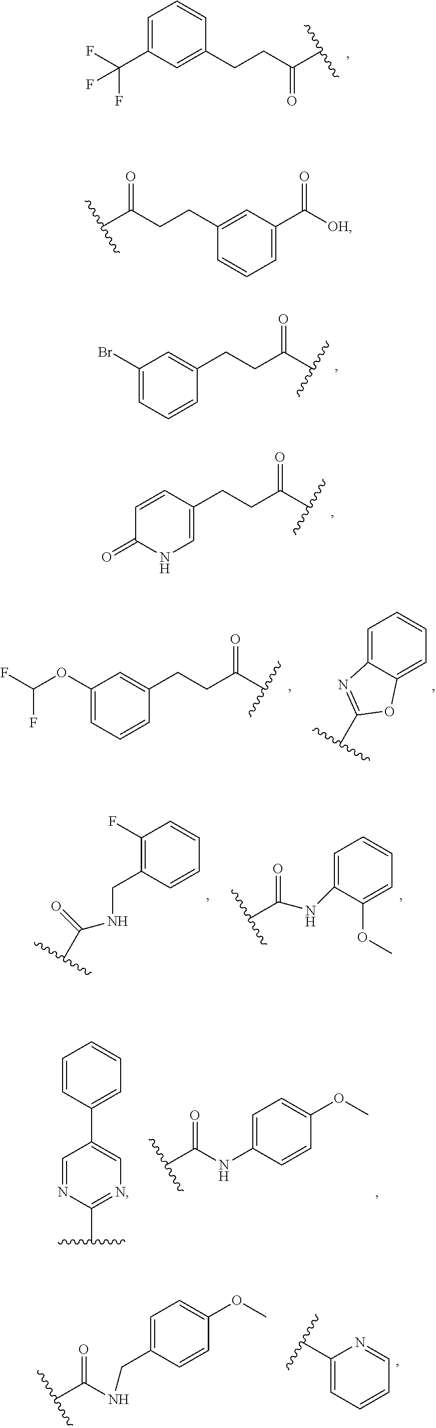

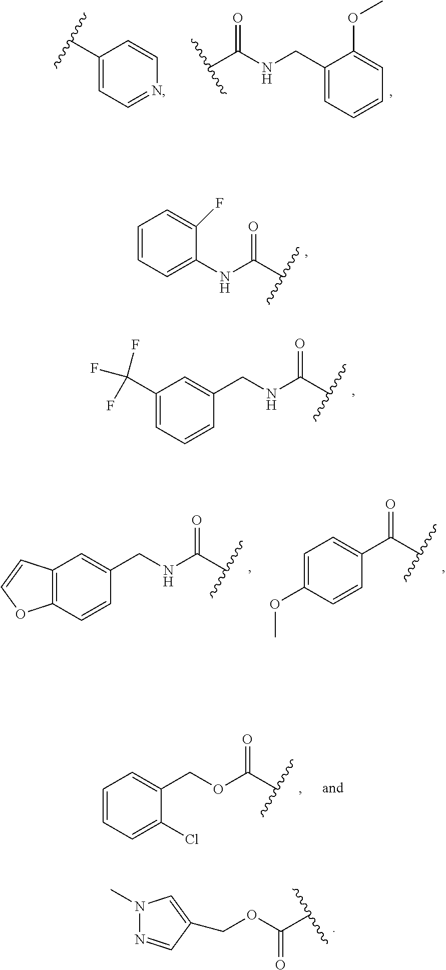

21. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof, wherein -V-Z- is selected from —CH₂—, —O—CH₂—, —CH₂—O—, —CH₂—CH₂—O—, —CH₂—N(R18)- and —N(R18)-CH₂— wherein R18 is selected from:
—(CH₂)₀₋₆-aryl, (CH₂)₀₋₆-heteroaryl^{a}, C(═O)R19, C(═O)OR19, C(═O)NHR19, C(═O)N(alkyl)(R19), and SO₂R19;
   wherein R19 is selected from (CH₂)₀₋₆-aryl and (CH₂)₀₋₆-heteroaryl^{a}.

22. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof, wherein -V-Z- is selected from —CH₂—, —O—CH₂—, —CH₂—O—, —CH₂—CH₂—O—, —CH₂—N(R18)- and —N(R18)-CH₂— wherein R18 is selected from:

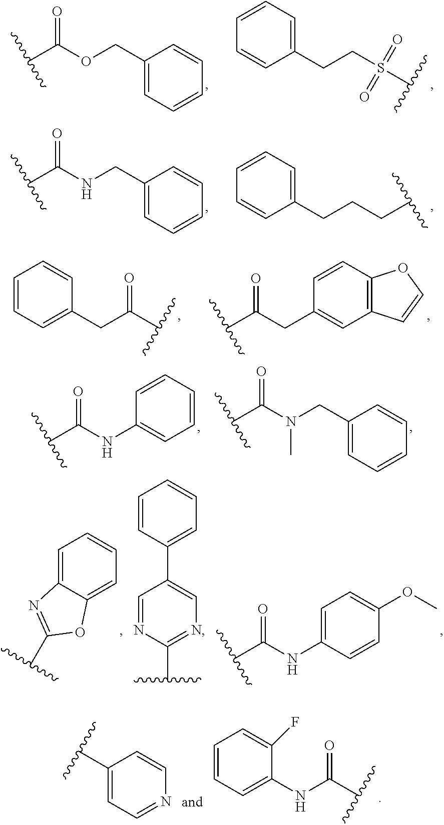

23. A compound of formula (I) according to numbered embodiment 18, or a tautomer, isomer, stereoisomer (including an enantiomer, a diastereoisomer and a racemic and scalemic mixture thereof), a deuterated isotope, and a pharmaceutically acceptable salt and/or solvate thereof, wherein -V-Z- is selected from —CH₂—, —O—CH₂—, —CH₂—O— and —CH₂—CH₂—O—.

24. A compound of formula (I) according to numbered embodiment 6, or a tautomer, isomer, stereoisomer (including an enantiomer, a diastereoisomer and a racemic and scalemic mixture thereof), a deuterated isotope, and a pharmaceutically acceptable salt and/or solvate thereof, wherein -V-Z- is selected from —O—CH₂— and —CH₂—O—.

25. A compound of formula (I) according to any of numbered embodiment 1, 2 or 3, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein:
U is absent;
X and Y are independently selected from O, CR1R2, C═O and NR12;
   wherein when one of X or Y is C═O, the other is O, CR1R2 or NR12;
   wherein when one of X or Y is NR12, the other is CR1R2 or C═O;
   wherein when one of X or Y is O, the other is CR1R2 or C═O;
-V-Z- is —CH₂— or;
V is O and Z is CR16R17;
which is a compound of formula (1d)

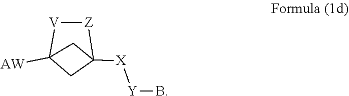

26. A compound of formula (I) according to any of numbered embodiments 1 to 3, or 6 to 23, or 25, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein U is absent and -V-Z- is —CH₂—
which is a compound of formula (1e)

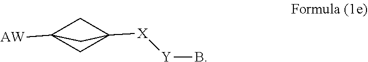

27. A compound of formula (I) according to any of numbered embodiments 1 to 3, or 25, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein U is absent, V is O and Z is CR16R17
which is a compound of formula (If)

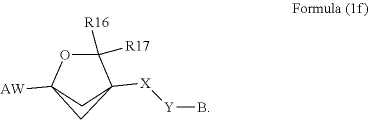

28. A compound of formula (I) according to numbered embodiment 27, or a tautomer, isomer, stereoisomer (including an enantiomer, a diastereoisomer and a racemic and scalemic mixture thereof), a deuterated isotope, and a pharmaceutically acceptable salt and/or solvate thereof, wherein R16 and R17 are both H, or R16 and R17 are both-CH₃.

29. A compound of formula (I) according to numbered embodiment 28, or a tautomer, isomer, stereoisomer (including an enantiomer, a diastereoisomer and a racemic and scalemic mixture thereof), a deuterated isotope, and a pharmaceutically acceptable salt and/or solvate thereof, wherein R16 and R17 are both H.

30. A compound of formula (I) according to any of numbered embodiments 1 to 24, or 26 to 29, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is selected from a bond and CR1R2.

31. A compound of formula (I) according to numbered embodiment 30, or a tautomer, isomer, stereoisomer (including an enantiomer, a diastereoisomer and a racemic and scalemic mixture thereof), a deuterated isotope, and a pharmaceutically acceptable salt and/or solvate thereof, wherein X is selected from a bond and CH₂.

32. A compound of formula (I) according to numbered embodiment 31, or a tautomer, isomer, stereoisomer (including an enantiomer, a diastereoisomer and a racemic and scalemic mixture thereof), a deuterated isotope, and a pharmaceutically acceptable salt and/or solvate thereof, wherein X is CH₂.

33. A compound of formula (I) according to numbered embodiment 31, or a tautomer, isomer, stereoisomer (including an enantiomer, a diastereoisomer and a racemic and scalemic mixture thereof), a deuterated isotope, and a pharmaceutically acceptable salt and/or solvate thereof, wherein X is CH₂, and Y is, where possible, N or NR12.

34. A compound of formula (I) according to numbered embodiment 31, or a tautomer, isomer, stereoisomer (including an enantiomer, a diastereoisomer and a racemic and scalemic mixture thereof), a deuterated isotope, and a pharmaceutically acceptable salt and/or solvate thereof, wherein X is CH₂, and Y is, where possible, N or NH.

35. A compound of formula (I) according to numbered embodiment 31, or a tautomer, isomer, stereoisomer (including an enantiomer, a diastereoisomer and a racemic and scalemic mixture thereof), a deuterated isotope, and a pharmaceutically acceptable salt and/or solvate thereof, wherein X is CH₂, Y is NR12, and U is absent.

36. A compound of formula (I) according to numbered embodiment 31, or a tautomer, isomer, stereoisomer (including an enantiomer, a diastereoisomer and a racemic and scalemic mixture thereof), a deuterated isotope, and a pharmaceutically acceptable salt and/or solvate thereof, wherein X is CH₂, Y is NH and U is absent.

37. A compound of formula (I) according to any of numbered embodiments 1 to 24, or 26 to 32, or a tautomer, isomer, stereoisomer (including an enantiomer, a diastereoisomer and a racemic and scalemic mixture thereof), a deuterated isotope, and a pharmaceutically acceptable salt and/or solvate thereof, wherein Y is, where possible, selected from O, CR1R2, N and NR12.

38. A compound of formula (I) according to numbered embodiment 37, or a tautomer, isomer, stereoisomer (including an enantiomer, a diastereoisomer and a racemic and scalemic mixture thereof), a deuterated isotope, and a pharmaceutically acceptable salt and/or solvate thereof, wherein Y is, where possible, selected from O, CH₂, N and NH.

39. A compound of formula (I) according to any of numbered embodiments 1 to 3, 13 to 24, or 30 to 38, or a tautomer, isomer, stereoisomer (including an enantiomer, a diastereoisomer and a racemic and scalemic mixture thereof), a deuterated isotope, and a pharmaceutically acceptable salt and/or solvate thereof, wherein:
-V-Z- is —CH₂—, X is CH₂ and Y is NH;
-V-Z- is —O—CH₂—, X is CH₂ and Y is NH;
-V-Z- is —CH₂—O—, X is CH₂ and Y is NH; or
-V-Z- is —CH₂—CH₂—O—, X is CH₂ and Y is NH.

40. A compound of formula (I) according to any of numbered embodiments 1 to 29, or 37 to 38, or a tautomer, isomer, stereoisomer (including an enantiomer, a diastereoisomer and a racemic and scalemic mixture thereof), a deuterated isotope, and a pharmaceutically acceptable salt and/or solvate thereof, wherein X is C═O.

41. A compound of formula (I) according to any of numbered embodiments 1 to 29, or 37 to 38, or a tautomer, isomer, stereoisomer (including an enantiomer, a diastereoisomer and a racemic and scalemic mixture thereof), a deuterated isotope, and a pharmaceutically acceptable salt and/or solvate thereof, wherein X is NR12.

42. A compound of formula (I) according to any of numbered embodiments 1 to 30, or 37 to 17, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is CR1R2.

43. A compound of formula (I) according to any of numbered embodiments 1 to 29, or 37 to 38, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is O.

44. A compound of formula (I) according to any one of numbered embodiments 1 to 23, 26 to 31, or 37 to 38, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is a bond.

45. A compound of formula (I) according to any of numbered embodiments 1 to 29, 32, or 41 to 43, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is, where possible, C═O.

46. A compound of formula (I) according to any of numbered embodiments 1 to 37, 40, 42, or 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is, where possible, NR12 or N.

47. A compound of formula (I) according to any of numbered embodiments 1 to 37, 40, 42, or 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is, where possible, NR12.

48. A compound of formula (I) according to any of numbered embodiments 1 to 30, 32 to 37, 40 to 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is, where possible, CR1R2.

49. A compound of formula (I) according to any of numbered embodiments 1 to 38, 40, 42 or 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is, where possible, O.

50. A compound of formula (I) according to any of numbered embodiments 1, 28 to 38, 40, 42 or 44, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is, where possible, N.

51. A compound of formula (I) according to numbered embodiment 41 or 47, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein NR12 is NH.

52. A compound of formula (I) according to numbered embodiment 41, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is NH.

53. A compound of formula (I) according to numbered embodiment 47, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is, where possible, NH.

54. A compound of formula (I) according to any of numbered embodiments 42 or 48, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R1 is H.

55. A compound of formula (I) according to numbered embodiment 42, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is CHR2.

56. A compound of formula (I) according to numbered embodiment 48, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is, where possible, CHR2.

57. A compound of formula (I) according to any of numbered embodiments 42, 48, or 54, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein R2 is H.

58. A compound of formula (I) according to numbered embodiment 42, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is CR1H.

59. A compound of formula (I) according to numbered embodiment 48, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is, where possible, CR1H.

60. A compound of formula (I) according to any of numbered embodiments 55 or 58, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein X is CH₂.

61. A compound of formula (I) according to any of numbered embodiments 56 or 59, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is, where possible, CH₂.

62. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from heteroaryl^{a} and aryl.

63. A compound of formula (I) according to numbered embodiment 62, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is heteroaryl^{a}.

64. A compound of formula (I) according to numbered embodiment 63 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is attached to B at a carbon atom on the heteroaryl^{a} ring.

65. A compound of formula (I) according to numbered embodiment 63 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein Y is attached to B at a carbon atom on the heteroaryl^{a} ring, and the two ring atoms adjacent to the carbon atom on the heteroaryl^{a} ring to which Y attaches are both carbon.

66. A compound of formula (I) according to any of numbered embodiments 63 to 65, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is a 9 or 10 membered bicyclic aromatic ring, containing, where possible, 1, 2, 3 or 4 ring members independently selected from N, NR12, S and O, optionally substituted as for heteroaryl^{a}.

67. A compound of formula (I) according to any of numbered embodiments 63 to 66, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is a 9 or 10 membered bicyclic aromatic ring, containing 1 or 2 ring members independently selected from N or NR12, optionally substituted as for heteroaryl^{a}.

68. A compound of formula (I) according to numbered embodiment 67, or a tautomer, isomer, stereoisomer (including an enantiomer, a diastereoisomer and a racemic and scalemic mixture thereof), a deuterated isotope, and a pharmaceutically acceptable salt and/or solvate thereof, wherein B is:
isoquinolinyl, optionally substituted as for heteroaryl^{a}, or
azaindole, optionally substituted as for heteroaryl^{a}.

69. A compound of formula (I) according to numbered embodiment 68 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl

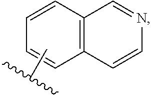

optionally substituted as for heteroaryl^{a};
6-azaindolyl

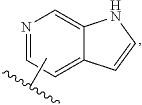

optionally substituted as for heteroaryl^{a}; and
7-azaindolyl

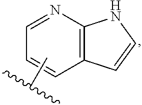

optionally substituted as for heteroaryl^{a}.

70. A compound of formula (I) according to numbered embodiment 69 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl

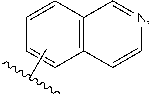

optionally substituted as for heteroaryl^{a}; and
7-azaindolyl

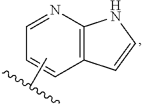

optionally substituted as for heteroaryl^{a}.

71. A compound of formula (I) according to numbered embodiment 69 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl, substituted with NH₂ at the 1-position

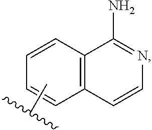

optionally further substituted with 1 or 2 substituents as for heteroaryl^{a};
6-azaindolyl

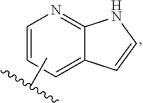

optionally substituted as for heteroaryl^{a}; and
7-azaindolyl

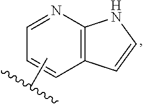

optionally substituted as for heteroaryl^{a}.

72. A compound of formula (I) according to numbered embodiment 68 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl, substituted with NH₂ at the 1-position

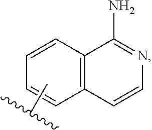

optionally further substituted with 1 or 2 substituents as for heteroaryl^{a}; and
7-azaindolyl

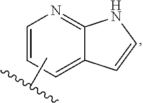

optionally substituted as for heteroaryl^{a}.

73. A compound of formula (I) according to numbered embodiment 68 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl, selected from

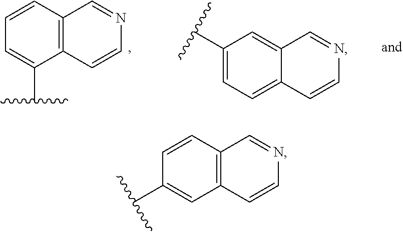

optionally substituted as for heteroaryl^{a};
7-azaindolyl

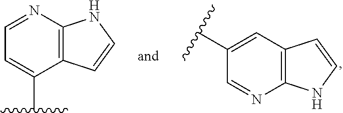 [SMILES: *c1ccnc2[nH]ccc12 ; *c1cnc2[nH]ccc2c1]

optionally substituted as for heteroaryl^{a}; and
6-azaindolyl

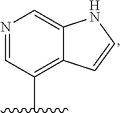 [SMILES: *c1cncc2[nH]ccc12]

optionally substituted as for heteroaryl^{a}.

74. A compound of formula (I) according to numbered embodiment 68 or a tautomer, isomer, stereoisomer (including an enantiomer, a diastereoisomer and a racemic and scalemic mixture thereof), a deuterated isotope, and a pharmaceutically acceptable salt and/or solvate thereof, wherein B is selected from:
isoquinolinyl, selected from

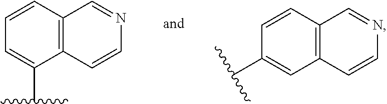 [SMILES: *c1cccc2cnccc12 ; *c1ccc2cnccc2c1]

optionally substituted as for heteroaryl^{a}; and
7-azaindolyl

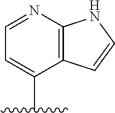 [SMILES: *c1ccnc2[nH]ccc12]

optionally substituted as for heteroaryl^{a}.

75. A compound of formula (I) according to numbered embodiment 68 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl, substituted with NH₂ at the 1-position, selected from

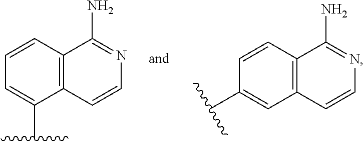 [SMILES: *c1cccc2c(N)nccc12 ; *c1ccc2c(N)nccc2c1]

optionally further substituted with 1 or 2 substituents as for heteroaryl^{a};
6-azaindolyl

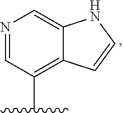 [SMILES: *c1cncc2[nH]ccc12]

optionally substituted as for heteroaryl^{a}; and
7-azaindolyl selected from

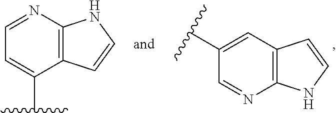 [SMILES: *c1ccnc2[nH]ccc12 ; *c1cnc2[nH]ccc2c1]

optionally substituted as for heteroaryl^{a}.

76. A compound of formula (I) according to numbered embodiment 68 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl, substituted with NH₂ at the 1-position, selected from

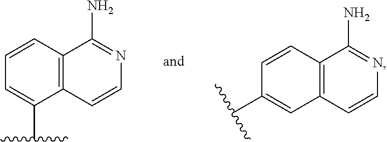 [SMILES: *c1cccc2c(N)nccc12 ; *c1ccc2c(N)nccc2c1]

optionally further substituted with 1 or 2 substituents as for heteroaryl^{a}; and
7-azaindolyl selected from

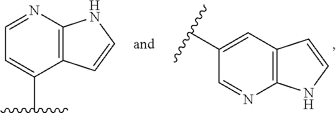 [SMILES: *c1ccnc2[nH]ccc12 ; *c1cnc2[nH]ccc2c1]

optionally substituted as for heteroaryl^{a}. 77. A compound of formula (I) according to numbered embodiment 68 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is selected from:
isoquinolinyl, substituted with NH₂ at the 1-position, selected from

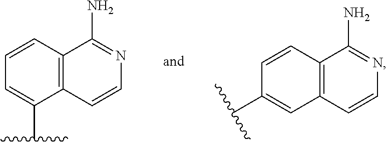 [SMILES: *c1cccc2c(N)nccc12 ; *c1ccc2c(N)nccc2c1]

optionally further substituted with 1 or 2 substituents as for heteroaryl^{a}; and
7-azaindolyl

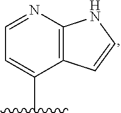 [SMILES: *c1ccnc2[nH]ccc12]

optionally substituted as for heteroaryl^{a}.

78. A compound of formula (I) according to numbered embodiment 68, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoquinolinyl, optionally substituted as for heteroaryl^{a}.

79. A compound of formula (I) according to numbered embodiment 78, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoquinolinyl, wherein B can be selected from

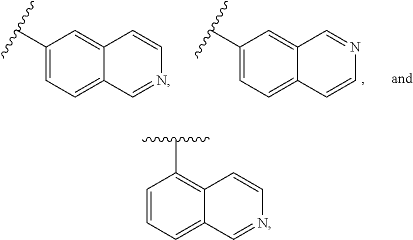 [SMILES: *c1ccc2cnccc2c1 ; *c1ccc2ccncc2c1 ; *c1cccc2cnccc12]

optionally substituted as for heteroaryl^{a}.

80. A compound of formula (I) according to numbered embodiment 79, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoquinolinyl, wherein B can be selected from

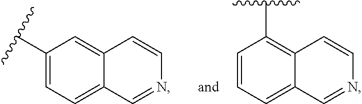 [SMILES: *c1ccc2cnccc2c1 ; *c1cccc2cnccc12]

optionally substituted as for heteroaryl^{a}.

81. A compound of formula (I) according to numbered embodiment 79, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is isoquinolinyl, wherein B can be

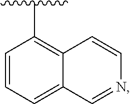 [SMILES: *c1cccc2cnccc12]

optionally substituted as for heteroaryl^{a}.

82. A compound of formula (I) according to numbered embodiment 79 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is:

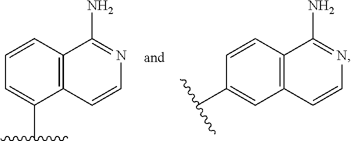 [SMILES: *c1cccc2c(N)nccc12 ; *c1ccc2c(N)nccc2c1]

isoquinolinyl, substituted with NH₂ at the 1-position, selected from optionally further substituted with 1 or 2 substituents as for heteroaryl^{a}.

83. A compound of formula (I) according to numbered embodiment 79 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is:
isoquinolinyl, substituted with NH₂ at the 1-position

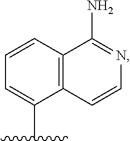 [SMILES: *c1cccc2c(N)nccc12]

optionally further substituted with 1 or 2 substituents as for heteroaryl^{a}.

84. A compound of formula (I) according to numbered embodiment 79 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is:
isoquinolinyl, substituted with NH₂ at the 1-position

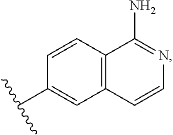 [SMILES: *c1ccc2c(N)nccc2c1]

optionally further substituted with 1 or 2 substituents as for heteroaryl^{a}.

85. A compound of formula (I) according to numbered embodiment 68, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is azaindole, optionally substituted as for heteroaryl^{a}.

86. A compound of formula (I) according to numbered embodiment 85, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein azaindole, selected from

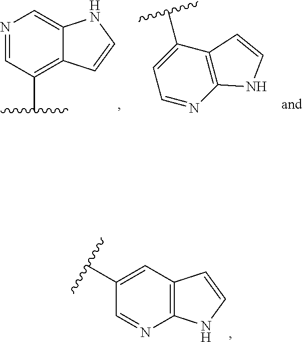

optionally substituted as for heteroaryl^{a}.

87. A compound of formula (I) according to numbered embodiment 85, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is 7-azaindole, optionally substituted as for heteroaryl^{a}.

88. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is 7-azaindole, selected from

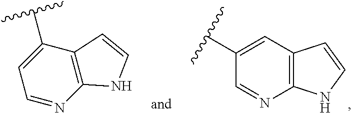 [SMILES: *c1ccnc2[nH]ccc12 ; *c1cnc2[nH]ccc2c1]

optionally substituted as for heteroaryl^{a}.

89. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is 7-azaindole

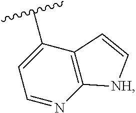 [SMILES: *c1ccnc2[nH]ccc12]

optionally substituted as for heteroaryl^{a}.

90. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof, wherein B is 7-azaindole substituted with methyl, and optionally substituted with 1 or 2 further substituents as for heteroaryl^{a}.

91. A compound of formula (I) according to numbered embodiment 85, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is azaindole, selected from

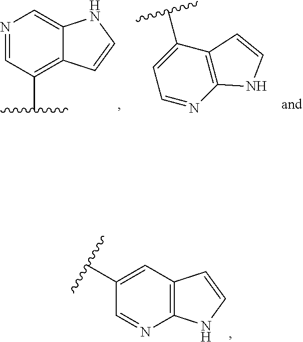

substituted with methyl, and optionally substituted with 1 or 2 further substituents as for heteroaryl^{a}.

92. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is 7-azaindole, selected from

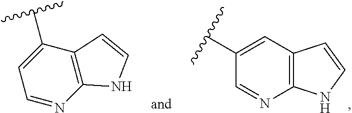 [SMILES: *c1ccnc2[nH]ccc12 ; *c1cnc2[nH]ccc2c1]

substituted with methyl, and optionally substituted with 1 or 2 further substituents as for heteroaryl^{a}.

93. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is 7-azaindole

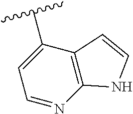 [SMILES: *c1ccnc2[nH]ccc12]

substituted with methyl, and optionally substituted with 1 or 2 further substituents as for heteroaryl^{a}.

94. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof, wherein B is 7-azaindole substituted with chloro, and optionally substituted with 1 or 2 further substituents as for heteroaryl^{a}.

95. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is 7-azaindole, B can be selected from

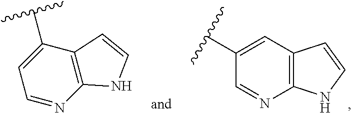 [SMILES: *c1ccnc2[nH]ccc12 ; *c1cnc2[nH]ccc2c1]

substituted with chloro, and optionally substituted with 1 or 2 further substituents as for heteroaryl^{a}.

96. A compound of formula (I) according to numbered embodiment 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is 7-azaindole

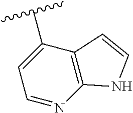 [SMILES: *c1ccnc2[nH]ccc12]

substituted with chloro, and optionally substituted with 1 or 2 further substituents as for heteroaryl^{a}.

97. A compound of formula (I) according to any of numbered embodiments 62 to 87, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with NH₂, and optionally substituted with 1 or 2 further substituents as for heteroaryl^{a}.

98. A compound of formula (I) according to any of numbered embodiments 66 to 97, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein B is substituted with halo, and, where possible, optionally substituted with 1 or 2 further substituents as for heteroaryl^{a}.

99. A compound of formula (I) according to numbered embodiment 98, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein the halo is C1.

100. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is selected from —(CHR12)-A, —(CH₂)₀₋₆—C(═O)—(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₃-(phenyl)-(CH₂)₀₋₃-A and —(CH₂)₀₋₆—NH—C(═O)—(CH₂)₀₋₆-A.

101. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is selected from —(CHR12)-A, —(CH₂)₀₋₆—C(═O)—(CH₂)₀₋₆-A, and —(CH₂)₀₋₆—O—(CH₂)₀₋₆-A.

102. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is —(CH₂)₀₋₆—O—(CH₂)₀₋₆-A.

103. A compound of formula (I) according to any preceding numbered embodiment, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is —CH₂—O-A.

104. A compound of formula (I) according to numbered embodiment 101, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is selected from —(CHR12)-A, and —(CH₂)₀₋₆—O—(CH₂)₀₋₆-A.

105. A compound of formula (I) according to numbered embodiment 104, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is —(CHR12)-A.

106. A compound of formula (I) according to numbered embodiment 105, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein —(CHR12)-A is —(CH₂)-A.

107. A compound of formula (I) according to numbered embodiment 104, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is —(CH₂)₀₋₆—O—(CH₂)₀₋₆-A.

108. A compound of formula (I) according to numbered embodiment 107, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is —(CH₂)—O—(CH₂)₀₋₆-A.

109. A compound of formula (I) according to numbered embodiment 107, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is —(CH₂)₀₋₆—O-A.

110. A compound of formula (I) according to any of numbered embodiments 104 or 107 to 58, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is —(CH₂)—O-A.

111. A compound of formula (I) according to numbered embodiment 107, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is —(CH₂)₀₋₆—O—(CH₂)-A.

112. A compound of formula (I) according to numbered embodiment 107, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is —O—(CH₂)₀₋₆-A.

113. A compound of formula (I) according to any of numbered embodiments 104, 107 or 110 to 111, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein AW- is —O—(CH₂)-A.

114. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is not:
   (iii)

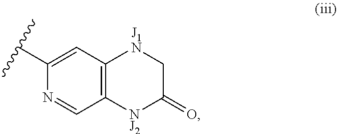

which may be optionally substituted at J₁, J₂, or any other ring position on A; or
   (iv)

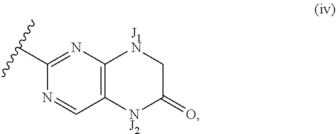

which may be optionally substituted at J₁, J₂, or any other ring position on A.

115. A compound of formula (I) according to any preceding numbered embodiment or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 4- to 12-membered mono- or bi-cyclic ring system, containing one N ring member and optionally one, two or three further ring members independently selected from N, O and S, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein when A is a bicyclic ring system, the bicyclic ring system is fused, bridged or spiro.

116. A compound of formula (I) according to numbered embodiment 115 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 4- to 12-membered mono- or bi-cyclic ring system, containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein when A is a bicyclic ring system, the bicyclic ring system is fused, bridged or spiro.

117. A compound of formula (I) according to numbered embodiment 116 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 9 or 10 membered bicyclic ring system containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein at least one of the rings forming the bicyclic ring system is aromatic.

118. A compound of formula (I) according to numbered embodiment 117 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 9 or 10 membered bicyclic ring system containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein both of the rings forming the bicyclic ring system are aromatic.

119. A compound of formula (I) according to any of numbered embodiments 117 to 118, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 9 membered bicyclic ring.

120. A compound of formula (I) according to numbered embodiment 116, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 6 or 7 membered mono- or bi-cyclic ring system, containing one N ring member and optionally one or two further ring members independently selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN;
wherein when A is a bicyclic ring system, the bicyclic ring system is fused, bridged or spiro.

121. A compound of formula (I) according to numbered embodiment 120, or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is a 6 membered monocyclic ring system containing one N ring member and optionally one further ring member selected from N and O, optionally wherein the ring system is substituted, where possible, with 1, 2, 3 or 4 substituents independently selected from halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, CN.

122. A compound of formula (I) according to numbered embodiment 115 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

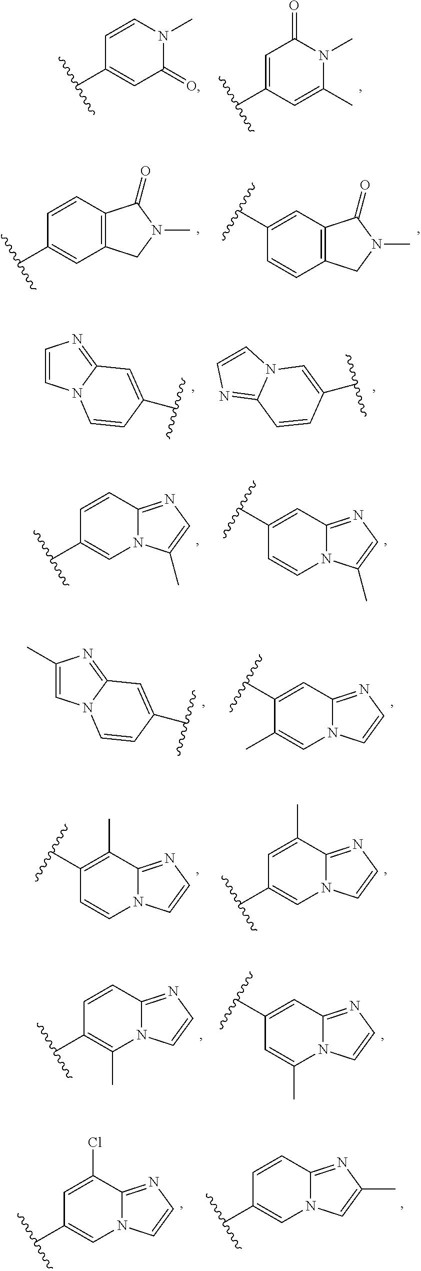

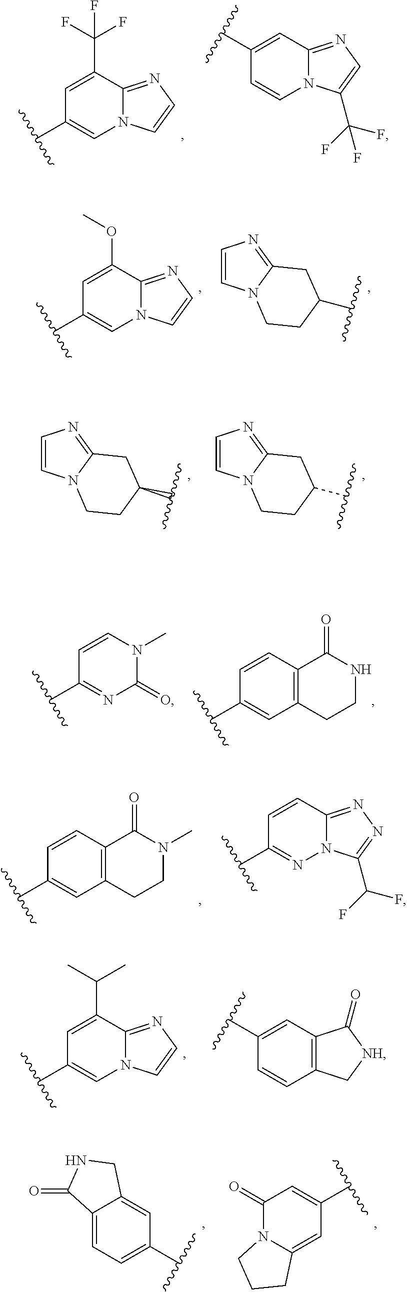

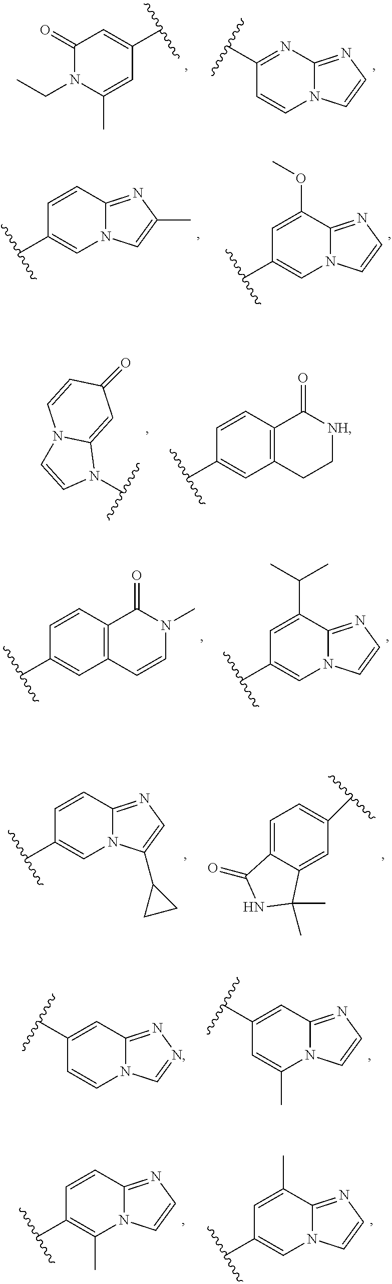

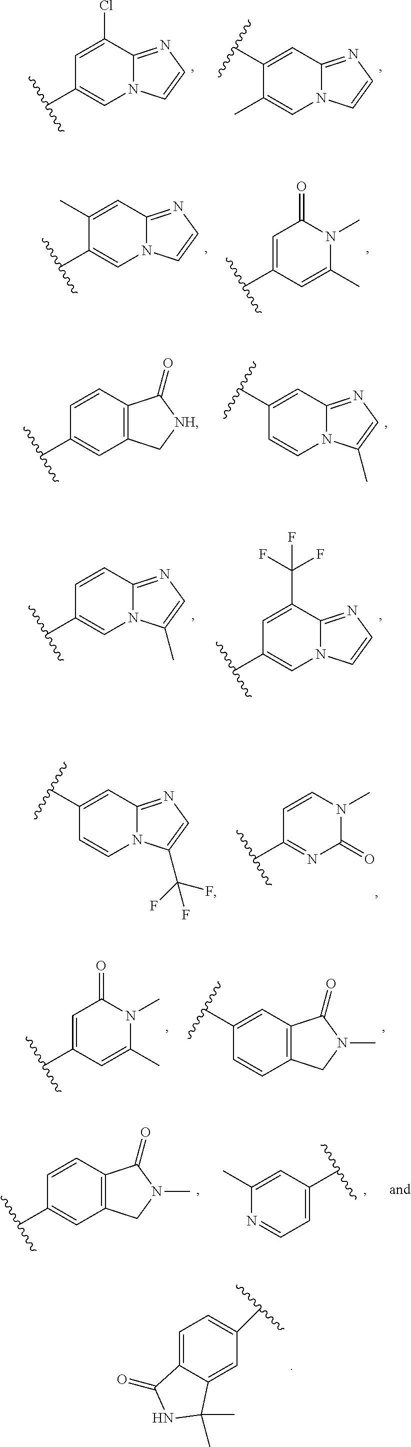

123. A compound of formula (I) according to numbered embodiment 115 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

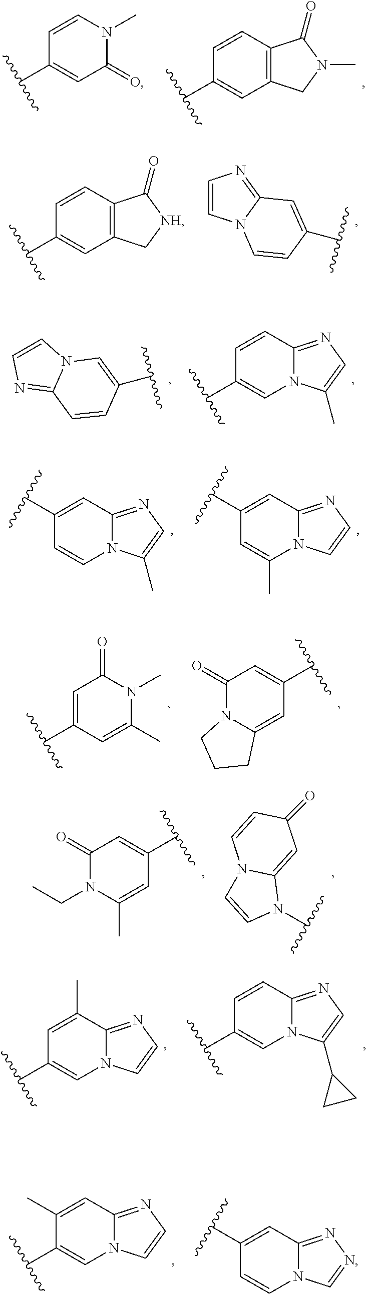

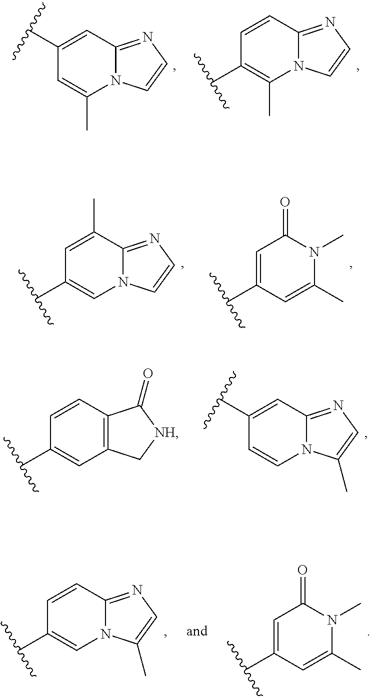

124. A compound of formula (I) according to numbered embodiment 115 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

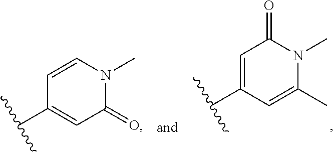

125. A compound of formula (I) according to numbered embodiment 116 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

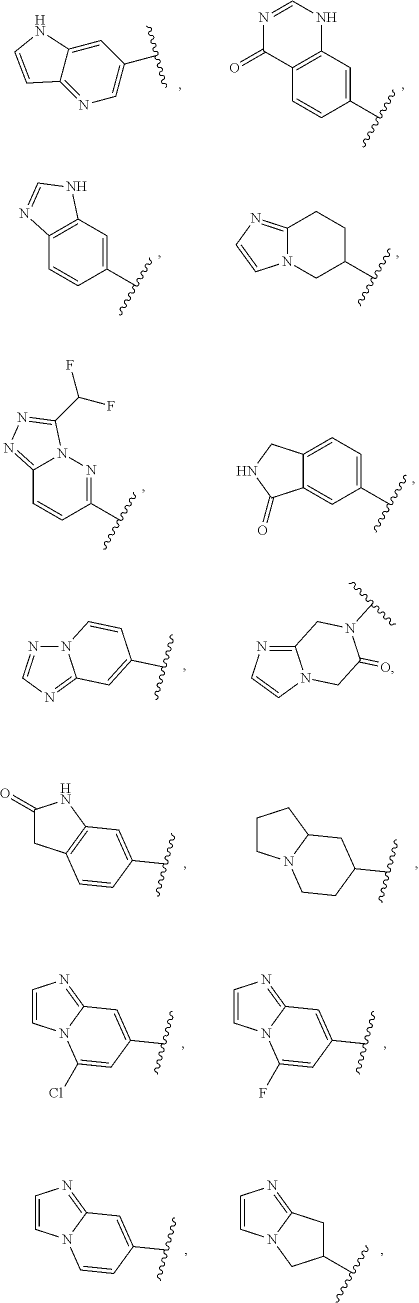

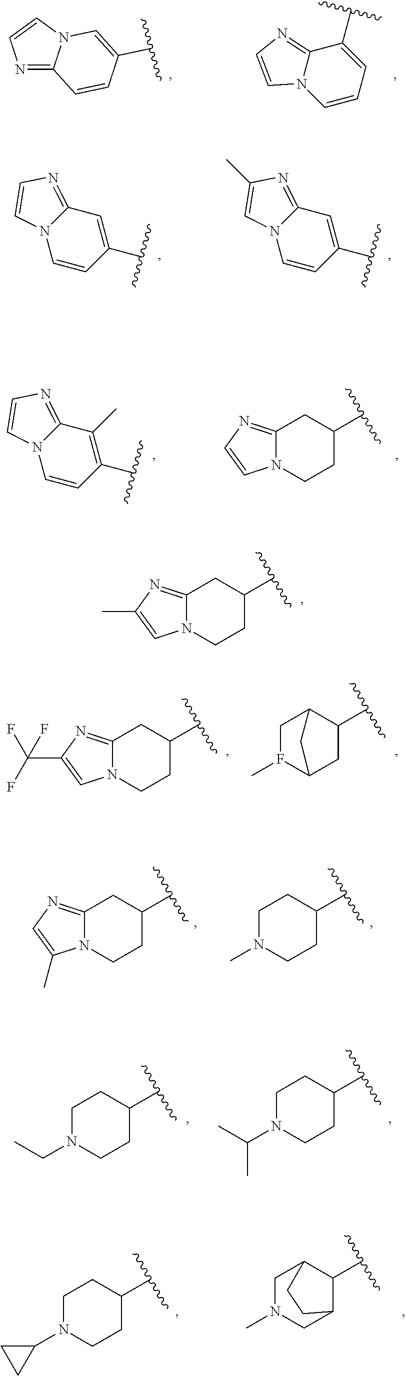

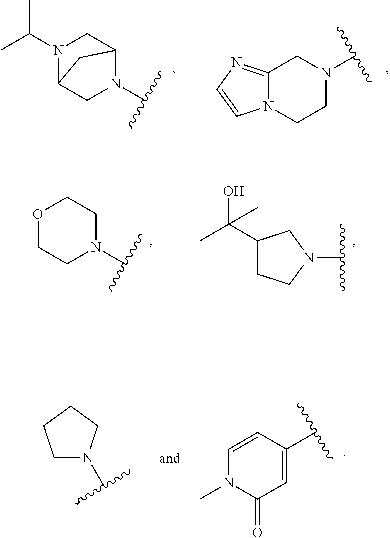

126. A compound of formula (I) according to numbered embodiment 125 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

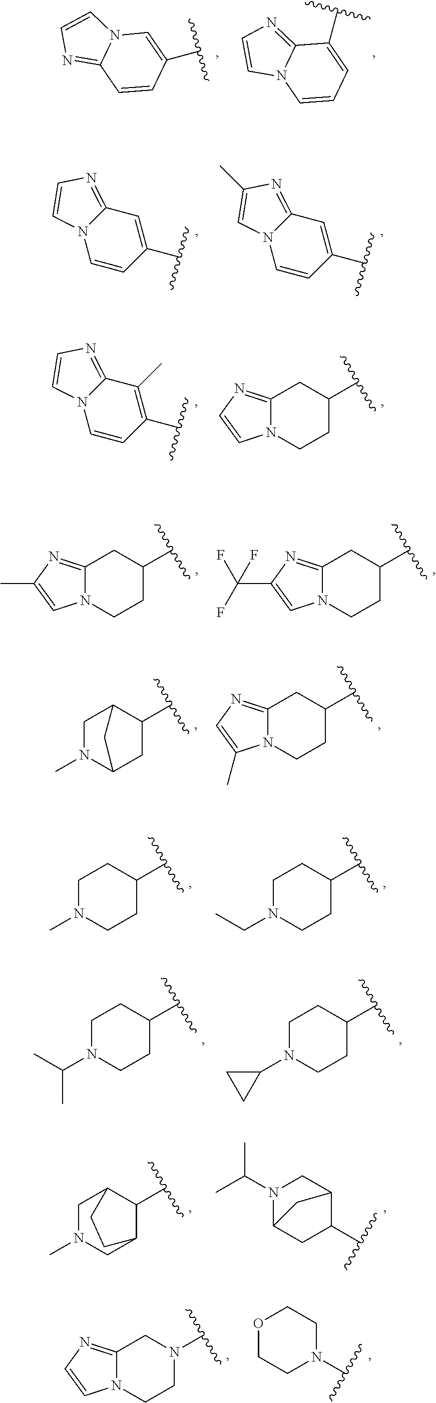

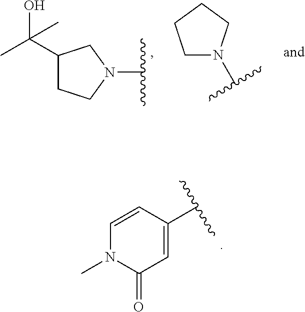

127. A compound of formula (I) according to numbered embodiment 126 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

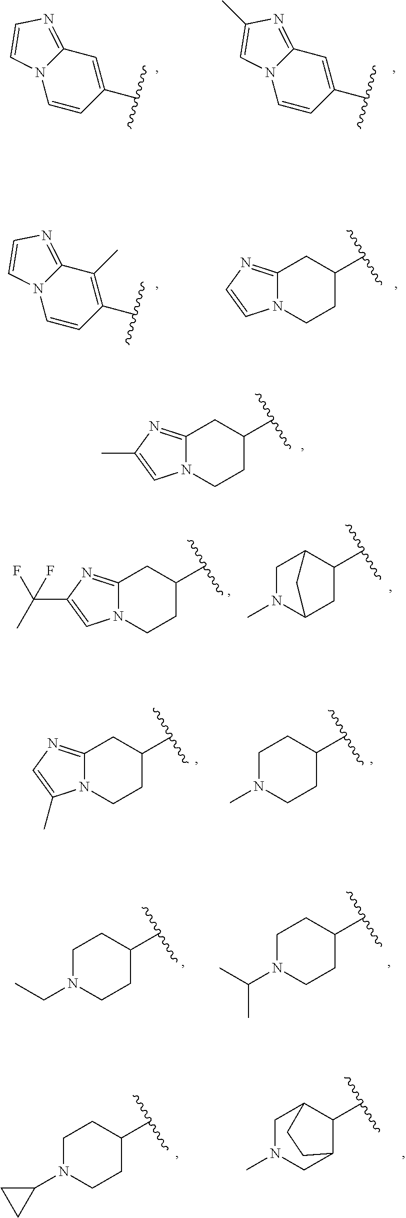

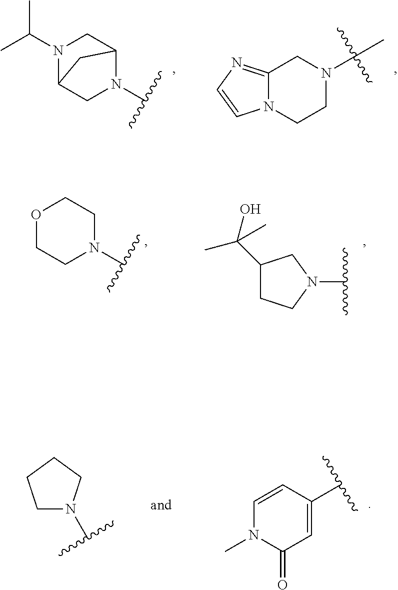

128. A compound of formula (I) according to numbered embodiment 116 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

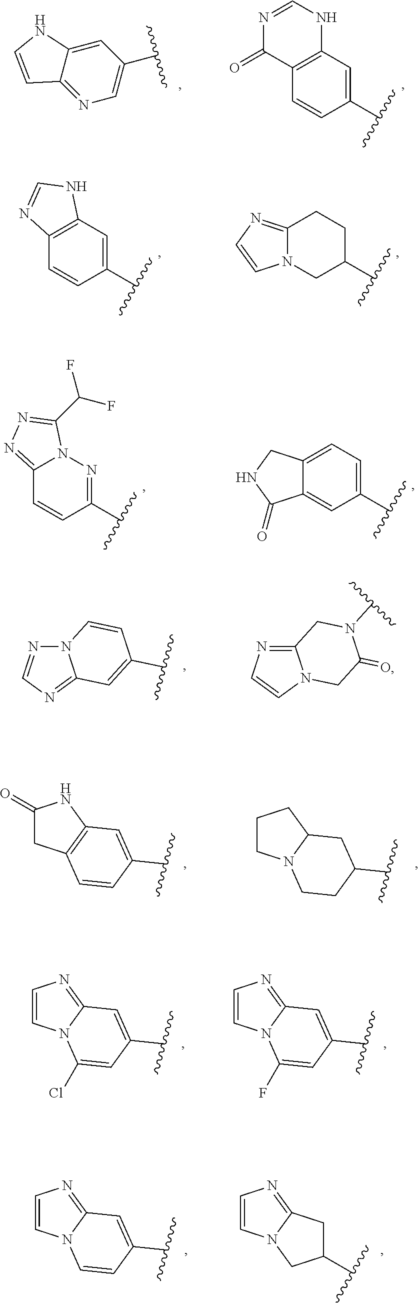

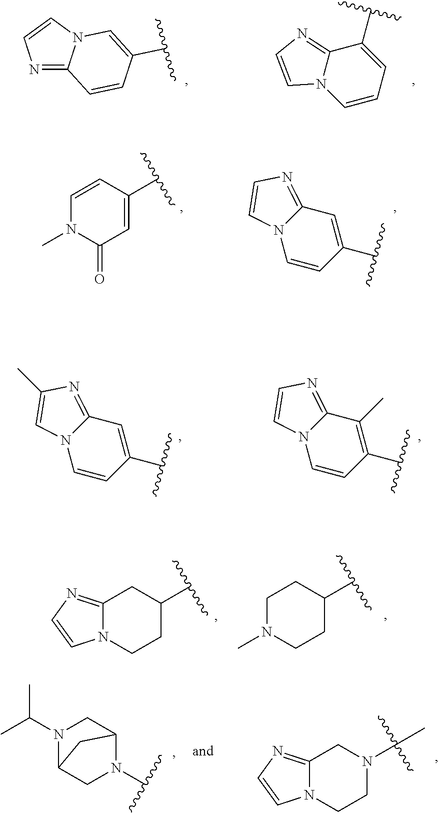

129. A compound of formula (I) according to numbered embodiment 128 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

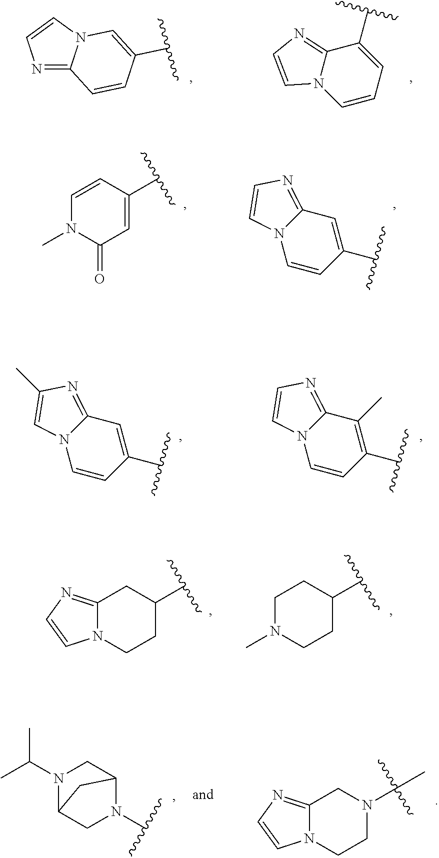

130. A compound of formula (I) according to numbered embodiment 129 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

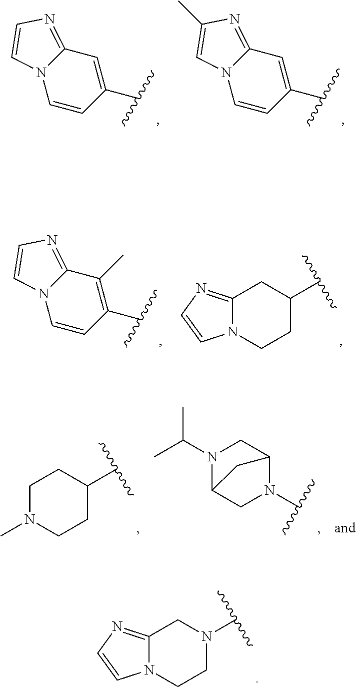

131. A compound of formula (I) according to numbered embodiment 125 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is selected from:

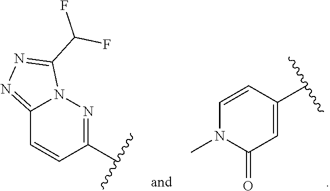

132. A compound of formula (I) according to numbered embodiment 131 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is:

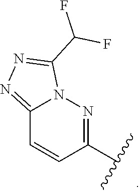 [SMILES: *c1ccc2nnc(C(F)F)n2n1]

133. A compound of formula (I) according to numbered embodiment 131 or a tautomer, isomer, stereoisomer (including an enantiomer, a diastereoisomer and a racemic and scalemic mixture thereof), a deuterated isotope, and a pharmaceutically acceptable salt and/or solvate thereof,
wherein A is:

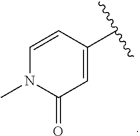 [SMILES: *c1ccn(C)c(=O)c1]

134. A compound selected from Table 1a, Table 1b, Table 2a, Table 2b, Table 3a, Table 4a, Table 5b, Table 6a, Table 6b, Table 7a, Table 7b, Table 8a, Table 8b, Table 9b, Table 10a, Table 10b, Table 11b, Table 12b, or Table 13b, or a pharmaceutically acceptable salt, solvate, or solvate of a salt thereof.

135. A compound selected from Table 1a, Table 2a, Table 3a, Table 4a, Table 6a, Table 7a, Table 8a, or Table 10a, or a pharmaceutically acceptable salt, solvate, or solvate of a salt thereof.

136. A compound selected from Example numbers 1001, 1002, 1002.1, 1002.2, 1003, 1004, 1005, 1005.1, 1005.2, 1006, 1007, 1008, 1009, 1010, 1011, 1012, 1013, 1014, 1015, 1016, 1017, 1018, 1019, 2020, 2021, 2022, 3023 or 3024, or a pharmaceutically acceptable salt, solvate, or solvate of a salt thereof.

137. A compound selected from Example numbers 1001, 1002, 1002.1, 1002.2, 1003, 1004, 1005, 1005.1, 1005.2, 1006, 1007, 1008, 1009, 1010 or 1011, or a pharmaceutically acceptable salt, solvate, or solvate of a salt thereof.

138. A pharmaceutical composition comprising: a compound, or a pharmaceutically acceptable salt and/or solvate thereof, according to any of numbered embodiments 1 to 137, and at least one pharmaceutically acceptable excipient.

139. A compound, or a pharmaceutically acceptable salt and/or solvate thereof, as defined in any of numbered embodiments 1 to 137, or the pharmaceutical composition according to numbered embodiment 138, for use in medicine.

140. A compound, or a pharmaceutically acceptable salt and/or solvate thereof, as defined in any of numbered embodiments 1 to 137, or the pharmaceutical composition according to numbered embodiment 138, for use in a method of treatment of a disease or condition in which Factor XIIa activity is implicated.

141. A compound, a pharmaceutically acceptable salt and/or solvate thereof, or a pharmaceutical composition for use as defined in numbered embodiment 140, wherein the disease or condition in which Factor XIIa activity is implicated is a bradykinin-mediated angioedema, wherein the bradykinin-mediated angioedema is hereditary angioedema.

142. A compound, a pharmaceutically acceptable salt and/or solvate thereof, or a pharmaceutical composition for use as defined in numbered embodiment 140, wherein the disease or condition in which Factor XIIa activity is implicated is a bradykinin-mediated angioedema, wherein the bradykinin-mediated angioedema is non hereditary.

143. A compound, a pharmaceutically acceptable salt and/or solvate thereof, or a pharmaceutical composition for use as defined in numbered embodiment 140, wherein the disease or condition in which Factor XIIa activity is implicated is selected from vascular hyperpermeability, stroke including ischemic stroke and hemorrhagic accidents; retinal edema; diabetic retinopathy; impaired visual acuity; DME; retinal vein occlusion; and AMD.

144. A compound, a pharmaceutically acceptable salt and/or solvate thereof, or a pharmaceutical composition for use as defined in numbered embodiment 140 wherein the disease or condition in which Factor XIIa activity is implicated is a thrombotic disorder.

145. A compound, a pharmaceutically acceptable salt and/or solvate thereof, or a pharmaceutical composition for use as defined in numbered embodiment 144, wherein the thrombotic disorder is thrombosis; thromboembolism caused by increased propensity of medical devices that come into contact with blood to clot blood; prothrombotic conditions such as disseminated intravascular coagulation (DIC), Venous thromboembolism (VTE), cancer associated thrombosis, complications caused by mechanical and bioprosthetic heart valves, complications caused by catheters, complications caused by ECMO, complications caused by LVAD, complications caused by dialysis, complications caused by CPB, sickle cell disease, joint arthroplasty, thrombosis induced to tPA, Paget-Schroetter syndrome and Budd-Chari syndrome; atherosclerosis; COVID-19; acute respiratory distress syndrome (ARDS); idiopathic pulmonary fibrosis (IPF); rheumatoid arthritis (RA); and cold-induced urticarial autoinflammatory syndrome.

146. A compound, a pharmaceutically acceptable salt and/or solvate thereof, or a pharmaceutical composition for use as defined in numbered embodiment 144, wherein the disease or condition in which Factor XIIa activity is implicated is neuroinflammation; neuroinflammatory/neurodegenerative disorders such as MS (multiple sclerosis); other neurodegenerative diseases such as Alzheimer's disease, epilepsy and migraine; sepsis; bacterial sepsis; inflammation; vascular hyperpermeability; and anaphylaxis.

147. A compound, a pharmaceutically acceptable salt and/or solvate thereof, or a pharmaceutical composition for use as defined in numbered embodiment 140, wherein the disease or condition in which Factor XIIa activity is implicated is obesity or diabetes.

### Synthetic Methods

The compounds of the present invention can be prepared according to the procedures of the following schemes and examples, using appropriate materials, and are further exemplified by the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. Those skilled in the art will readily understand that known variations of the conditions, processes and order in which the synthetic steps are performed in the following preparative procedures can be used to prepare these compounds.

The compounds and intermediates of the invention may be isolated in the form of their pharmaceutically acceptable salts, such as those described previously herein above. The interconversion between free form and salt form would be readily known to those skilled in the art.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 4^{th} Edition, 2006, may be used. For example, a common amino protecting group suitable for use herein is tert-butoxy carbonyl (boc), which is readily removed by treatment with an acid such as trifluoroacetic acid or hydrogen chloride in an organic solvent such as dichloromethane. Alternatively the amino protecting group may be a benzyloxycarbonyl (Cbz or Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvent. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively a trichloroethyl ester protecting group is removed with zinc in acetic acid. A common hydroxy protecting group suitable for use herein is a methyl ether, deprotection conditions comprise refluxing in 48% aqueous HBr, or by stirring with borane tribromide in an organic solvent such as DCM. Alternatively where a hydroxy group is protected as a benzyl ether, deprotection conditions comprise hydrogenation with a palladium catalyst under a hydrogen atmosphere.

The graphic representations of racemic, ambiscalemic and scalemic or enantiomerically pure compounds used herein are taken from Maehr J. Chem. Ed. 62, 114-120 (1985): solid wedges ( [symbol] ) and broken wedges ( [symbol] ) are used to denote the absolute configuration of a chiral element; wavy lines ( [symbol] ) indicate disavowal of any stereochemical implication which the bond it represents could generate; solid bold lines ( [symbol] ) and broken bold lines ( [symbol] ) are geometric descriptors indicating the relative configuration shown, but denoting racemic character; and wedge outlines ( [symbol] ) and broken lines ( [symbol] ) denote enantiomerically pure compounds of indeterminate absolute configuration. For nomenclature in the text corresponding to wedge outlines ( [symbol] ) and broken lines ( [symbol] ) we define R* and S* as indicating single enantiomers of uncertain absolute configuration. Additionally for nomenclature in the text corresponding to wedge outlines ( [symbol] ) and broken lines ( [symbol] ) which results in cis and trans isomers about a constrained ring system, we define cis* and trans* as indicating isomers of uncertain configuration.

Thus, for example, in examples 1002.1 and 1002.2 below, the synthesis of (S*)-N5-((3-(((5,6,7,8-tetrahydroimidazo[1,2-a]pyridin-7-yl)oxy)methyl)bicyclo[1.1.1]pentan-1-yl)methyl)isoquinoline-1,5-diamine and (R*)-N5-((3-(((5,6,7,8-tetrahydroimidazo[1,2-a]pyridin-7-yl)oxy)methyl)bicyclo[1.1.1]pentan-1-yl)methyl)isoquinoline-1,5-diamine are described. The (R*) and (S*) are intended to indicate that the product is a single enantiomer possessing the characteristics described (eq. NMR, HPLC, retention time etc), in which each of the chiral centres is believed on the basis of circumstantial evidence to be of the configuration shown, but the absolute configuration has not been confirmed. Thus, for example compound 1002.1, the depiction:

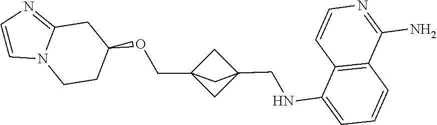 [SMILES: Nc1nccc2c(NCC34CC(CO[C@H]5CCn6ccnc6C5)(C3)C4)cccc12]

means that the compound is a single one of the following two stereoisomers, and probably the first:

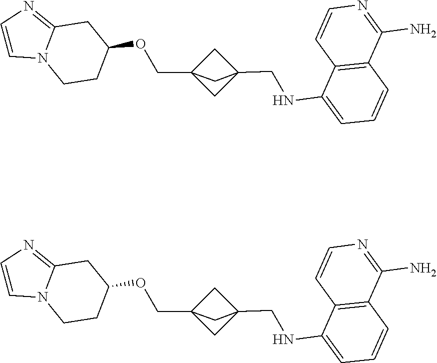 [SMILES: Nc1nccc2c(NCC34CC(CO[C@H]5CCn6ccnc6C5)(C3)C4)cccc12 ; Nc1nccc2c(NCC34CC(CO[C@@H]5CCn6ccnc6C5)(C3)C4)cccc12]

Additionally, for example, in examples 8806 and 8807 below, the synthesis of 5-(trans*-2-((imidazo[1,2-a]pyridin-7-yloxy)methyl)-6-azaspiro[3.4]octan-6-yl)isoquinolin-1-amine and 5-(cis*-2-((imidazo[1,2-a]pyridin-7-yloxy)methyl)-6-azaspiro[3.4]octan-6-yl)isoquinolin-1-amine are described. The trans* and cis* are intended to indicate that the product is a single isomer possessing the characteristics described (eq. NMR, HPLC, retention time etc), in which each of the combination of the chiral centres is believed on the basis of circumstantial evidence to be of the configuration shown, but the configuration has not been confirmed. Thus, for example compound 8806, the depiction:

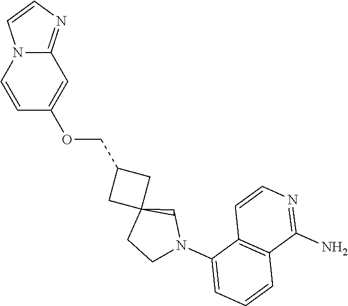

means that the compound is a single one of the following two isomers, and probably the first:

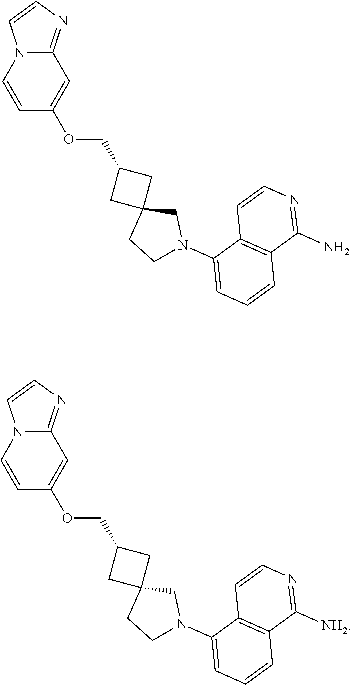 [SMILES: Nc1nccc2c(N3CC[C@]4(C3)C[C@H](COc3ccn5ccnc5c3)C4)cccc12 ; Nc1nccc2c(N3CC[C@]4(C3)C[C@@H](COc3ccn5ccnc5c3)C4)cccc12]

As used herein, a depiction including wedges or broken lines

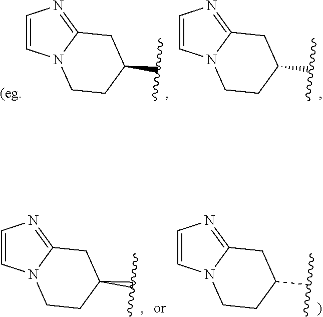

indicates that the structure encompasses purity of that relative or absolute configuration of at least 80% ee, preferably >90% ee.

As used herein, when a compound possesses a centre of asymmetry, its depiction with simple lines

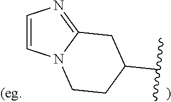 [SMILES: *C1CCn2ccnc2C1]

indicates that the structure includes any and all stereoisomers, without regard to enantiomeric purity.

The invention is illustrated by the following non-limiting examples in which the following abbreviations and definitions are used:

| | |
|---|---|
| AcOH | acetic acid |
| aq | aqueous solution |
| AIBN | azobisisobutyronitrile |
| boc | tert-butoxy carbonyl |
| Boc₂O | di-tert-butyl dicarbonate |
| BrettPhos Pd G3 | [(2-di-cyclohexylphosphino-3,6-dimethoxy-2′,4′,6′-triisopropyl-1,1′- |
| | biphenyl)-2-(2′-amino-1,1′-biphenyl)]palladium(II) methanesulfonate |
| BrettPhos Pd G4 | (SP-4-3)-[dicyclohexyl[3,6-dimethoxy-2′,4′,6′-tris(1-methylethyl)[1,1′- |
| | biphenyl]-2-yl]phosphine-κP](methanesulfonato-κO)[2′-(methylamino- |
| | κN)[1,1′-biphenyl]-2-yl-κC]palladium (CAS no. 1599466-83-7) |
| tBu | tert-butyl |
| tert-BuBrettphos | [(2-di-tert-butylphosphino-3,6-dimethoxy-2′,4′,6′-triisopropyl-1,1′- |
| Pd G3 | biphenyl)-2-(2′-amino-1,1′-biphenyl)]palladium(II) methanesulfonate |
| Cbz | benzyl carbamate |
| CDI | 1,1′-carbonyldiimidazole |
| Celite ® | Filter agent (diatomaceous earth) |
| DCM | dichloromethane |
| DIAD | diisopropyl azodicarboxylate |
| DIPEA | N,N-diisopropylethylamine |
| DMF | N,N-dimethylformamide |
| DMSO | dimethyl sulfoxide |
| EDC | 1-ethyl-3-(3-dimethylaminopropyl)carbodiimide hydrochloride |
| eq | equivalent |
| Et₂O | diethyl ether |
| Et | ethyl |
| EtOH | ethanol |
| EtOAc | ethyl acetate |
| HATU | 2-(3H-[1,2,3]triazolo[4,5-b]pyridin-3-yl)-1,1,3,3-tetramethylisouronium |
| | hexafluorophosphate(V) |
| h | Hours |
| HOBt | 1-hydroxybenzotriazole hydrate |
| IPA | isopropyl alcohol |
| LCMS | Liquid chromatography mass spectrometry |
| LiHMDS | lithium hexamethyldisilazide |
| Me | methyl |
| MeCN | acetonitrile |
| MeOH | methanol |
| min | minutes |
| MS | mass spectrum |
| Ms | methanesulfonyl |
| MsCl | methanesulfonyl chloride |
| NBS | N-bromosuccinimide |
| NCS | N-chlorosuccinimide |
| NMR | nuclear magnetic resonance spectrum |
| NMP | N-methyl-2-pyrrolidone |
| OAc | acetate |
| Pet. Ether | petroleum ether fraction boiling at 60-80° C. |
| Ph | phenyl |
| iPr | iso-propyl |
| nPr | n-propyl |
| RockPhos Pd G3 | [(2-di-tert-butylphosphino-3-methoxy-6-methyl-2′,4′,6′-triisopropyl- |
| | 1,1′-biphenyl)-2-(2-aminobiphenyl)]palladium(II) methanesulfonate |
| RuPhos Pd G3 | (2-dicyclohexylphosphino-2′,6′-diisopropoxy-1,1′-biphenyl)[2-(2′-amino- |
| | 1,1′-biphenyl)]palladium(II) methanesulfonate |
| sat. | saturated |
| SCX | strong cation exchange cartridge |
| STAB | sodium triacetoxyborohydride |
| SWFI | sterile water for injection |
| rt | room temperature |
| TBAB | tetra-n-butylammonium bromide |
| TBAF | tetra-n-butylammonium fluoride |
| TBDMS | tert-butyldimethylsilyl |
| TBME | tert-butyl methyl ether |
| THF | tetrahydrofuran |
| TEA | triethylamine |
| TFA | trifluoroacetic acid |
| Z | benzyl carbamate |

All reactions were carried out under an atmosphere of nitrogen unless specified otherwise.

Hydrogenations were typically carried out using an H-Cube® reactor (manufactured by Thalesnano, Inc, Hungary).

References to the use of microwave, a microwave reactor, microwave heating and microwave irradiation all refer to the use of a CEM Discover Microwave Reactor.

References to the use of a phase separator refer to columns fitted with a selectively permeable, optimized frit material that separates aqueous phase from an organic phase under gravity.

¹H NMR spectra were recorded using instrumentation selected from
Bruker (500 MHz or 400 MHz) spectrometer with a Bruker Avance II or Avance III console
Oxford (400 MHz) AS400 magnet with a Inova console
   and reported as chemical shift (ppm). It will be understood that, where exchangeable protons are present in any compound, the number of protons in the ¹H NMR spectra may not exactly correspond to the number of protons in the structure of any compound synthesised herein.

Molecular ions were obtained using LCMS with appropriate conditions selected from
Chromolith Speedrod RP-18e column, 50×4.6 mm, with a linear gradient 10% to 90%0.1% HCO₂H/MeCN into 0.1% HCO₂H/H₂O over 13 min, flow rate 1.5 mL/min;
Agilent, X-Select, acidic, 5-95% MeCN/water over 4 min. Data was collected using a Thermofinnigan Surveyor MSQ mass spectrometer with electrospray ionisation in conjunction with a Thermofinnigan Surveyor LC system;
LCMS (Waters Acquity UPLC, C18, Waters X-Bridge UPLC C18, 1.7 μm, 2.1×30 mm, Basic (0.1% Ammonium Bicarbonate) 3 min method;
LCMS (Agilent, X-Select, Waters X-Select C18, 2.5 μm, 4.6×30 mm, Acidic 4 min method, 95-5 MeCN/water);
LCMS (Agilent, Basic, Waters X-Bridge C18, 2.5 μm, 4.6×30 mm, Basic 4 min method, 5-95 MeCN/water;
Acquity UPLC BEH C18 1.7 UM column, 50×2.1 mm, with a linear gradient 10% to 90%0.1% HCO2H/MeCN into 0.1% HCO2H/H2 over 3 min, flow rate 1 mL/min. Data was collected using a Waters Acquity UPLC mass spectrometer with quadropole dalton, photodiode array and electrospray ionisation detectors;
Agilent 1100 LC/MSD with Kinetex® 5 μm EVO C18 100 Å LC 50×4.6 mm and Gemini® 5 μm NX-C18 110 Å LC 50×4.6 mm columns. Acidic mobile phases used a linear gradient of 5-95%10 mM aq. NH₄HCO₂/MeCN and basic mobile phases used a linear gradient of 5-95% 10 mM aq. NH₄HCO₃/MeCN. Samples were run over 3 minutes, using a flow rate of 2.2 mL/min, and a pressure range of 0-200 bar. Data was collected using a Waters 3100 Mass Detector with single high resolution quadrupole and photomultiplier detectors and High Performance Zspray™ dual-orthoganal API sources for standard ESI, or multimode ESI/APCI/ESCi®.
UPLC (CSH C18 Column, 130 Å, 1.7 μm, 2.1 mm×30 mm, 3 min method, 0.1% Formic acid, 2-100% MeCN/water)
LCMS (Cortecs C18+, 90 Å, 2.7 μm, 2.1 mm×30 mm, 3 min method, 0.1% Formic acid, 5-100% MeCN/water)
UPLC (BEH C18 Column, 130 Å, 1.7 μm, 2.1 mm×30 mm, 3 min method, 0.1% Ammonium Hydroxide, 2-100% MeCN/water)
LCMS (Kinetex Evo C18, 130 Å, 2.5 μm, 2.1 mm×30 mm, 3 min method, 0.1% Ammonium Hydroxide, 5-100% MeCN/water)

Flash chromatography was typically carried out over ‘silica’ (silica gel for chromatography, 0.035 to 0.070 mm (220 to 440 mesh) (e.g. Merck silica gel 60)), and an applied pressure of nitrogen up to 10 p.s.i accelerated column elution. Alternatively, pre-prepared cartridges of silica gel were used, for example pre-packed SiliaSep™ columns from Silicycle or Sfär C18 D-Duo 100 Å 30 μm columns from Biotage. Typical conditions included, for example, flow rate range of 18-200 ml/min, with an applied pressure range of 0-225 PSI. Hexanes, EtOAc, DCM, MeOH, were used as mobile phases for normal-phase chromatography purifications. MeOH, MeCN, 10 mM Ammonium Formate pH 4 in H₂O and 10 mM Ammonium Bicarbonate pH 10 in H₂O buffers were used as mobile phases for reverse-phase chromatography. It will be understood that alternative conditions (such as flow rate ranges, applied pressures, solvents and pH) may be used for flash chromatography in order to separate and purify compounds synthesised herein.

The term “prep HPLC” refers to reverse phase preparative HPLC purifications. Typical instrumentation and conditions included, for example, Agilent 1100/1200 Series Prep-HPLC with MWD/DAD & MSD using 5-100% acetonitrile/methanol with 10 mM NH₄HCO₃ pH 10 in H₂O buffer as a basic mobile phase and 5-100% acetonitrile/methanol with 10 mM NH₄HCO₂ pH 4 in H₂O, 0.1% TFA in H₂O, or HFBA Buffer in H₂O as an acidic mobile phase. It will be understood that alternative conditions (such as choice of column, flow rate ranges, solvents and pH) may be used for prep HPLC in order to separate and purify compounds synthesised herein.

The procedure of lyophilisation (or freeze drying) is generally well known in the art. Typically the substance is taken up in water, if necessary with the addition of a minimum amount of MeCN to aid dissolution, and frozen, typically by rapid cooling in a cold bath at −78° C. The resulting frozen solid mixture is evaporated to dryness in vacuo.

The term “concentrated” refers to evaporation of solvent under reduced pressure using a rotary evaporator, heating where necessary.

All solvents and commercial reagents were used as received.

IUPAC chemical names were generated using automated software such as Dotmatics Studies Notebook or ChemDraw (PerkinElmer). Compounds produced by the methods below may be isolated in salt forms.

However, compound naming used herein typically refers to the compound without any salt counter ion.

The example compounds described herein can be prepared using conventional synthetic methods for example, but not limited to, the routes outlined in the General Schemes below, using, for example, the General Methods below.

### General Methods

### 1. General Method 1: (GM1): Mesylation and Chlorination

a. General Method 1a (GM1a): Mesylation

A solution of alcohol (1.0 eq) in DCM (20 mL) was cooled in an ice/water bath and methane sulfonyl chloride (1.2 eq) was added dropwise followed by TEA (1.4 eq) maintaining cooling. The reaction was stirred at rt for 2-18 h. The reaction was diluted with DCM and washed with water. The aqueous layer was extracted with DCM (3×25 mL) and the combined organics were washed with brine, dried (Na₂SO₄), filtered and concentrated. The crude product was purified by flash chromatography.

b. General Method 1b (GM1b): Chlorination Via a Mesylate

Methane sulfonyl chloride (2.5 eq) (0.6 mL, 8.32 mmol) was added to a solution of TEA (2.8 eq) and alcohol (1.0 eq) in DCM (20 mL) while cooling in an ice/water bath. The reaction was stirred at rt for 18 h. The reaction was diluted with DCM and washed with sat. NaHCO₃ (aq). The aqueous layer was extracted with DCM (3×25 mL) and the combined organics were washed with brine, dried (Na₂SO₄), filtered and concentrated. The crude product was purified by flash chromatography.

### C. General Method 1c (GM1c): Chlorination Via NCS

A solution of indole or azaindole (1.0 eq) in DCM was protected from light and treated with NCS (3.75 eq) at rt for 12-48 h. The mixture was treated with 1M HCl (aq) and the phases separated. The organic phase was washed with brine, dried (Na₂SO₄), filtered, concentrated and purified by flash chromatography.

### 2. General Method 2 (GM2): SN₂ Alkylation (O and N)

a. General Method 2a: SN₂ Alkylation: NaH

To a suspension of NaH (60% wt. on mineral oil) (1.1 eq) in DMF in an ice/water bath was added a solution of alcohol, pyrrole or indole (1.0 eq) in DMF dropwise over 2 min. The mixture was allowed to warm to rt for 5 min before cooling again in an ice/water bath and treating with a solution of the alkylhalide or mesylate (1.0 eq) in DMF over 2 min. The mixture was maintained in an ice/water bath for 1 h before being allowed to warm to rt, or heated at elevated temperature and stirred for 2-18 h. Sat. NH₄Cl (aq) (50 mL) or sat. NaHCO₃ (aq) was added and extracted with EtOAc (×3). The organic phases were combined, dried (MgSO₄), filtered and concentrated. The crude product was purified by flash chromatography.

b. General Method 2b: SN₂ Alkylation; Cs₂CO₃ or K₂CO₃

A solution of alkyl halide or mesylate (1-2 eq), amine (1.0 eq), and base such as K₂CO₃, or CS₂CO₃ (2.5 eq), in a solvent such as DMF, DMSO or MeCN, was stirred at 80° C. for 2-18 h. MeOH (5 mL) was added and the mixture was diluted with water (50 mL). The product was extracted into EtOAc (2×50 mL) and washed with brine (50 mL). The organic layer was dried (Na₂SO₄), filtered and concentrated. The product was either used directly or purified by flash chromatography.

### 3. General Method 3 (GM3): Reduction

a. General Method 3a (GM3a): Nitrile Reduction; H-Cube® with Pd/C or Raney Ni Cartridge

The nitrile was dissolved in a 0.5M NH₃/MeOH solution passed through an H-Cube® reactor (Pd/C or Raney Ni cartridge), typical conditions: 50° C., ‘full’ hydrogen delivery mode (50 bar), flow rate: 1 mL/min. The reaction was concentrated to afford the product which was used without further purification.

b. General Method 3b (GM3b): Nitrile, Amide and Ester Reduction; LiAlH₄ in THF

To a solution of amide, nitrile, or ester (1.0 eq) in THF in an ice/water bath was added LiAlH₄ (2M in THF) (2.0 eq) dropwise and the reaction mixture was allowed to warm to rt then stirred for 4-18 h. The reaction mixture was cooled in an ice/water bath, treated portionwise with Na₂SO₄. 10H₂O (3.5 eq) and stirred for 30 min before being dried (MgSO₄), filtering and washing with THF (10 mL). The filtrate was concentrated to afford the crude product which was used without purification or purified by flash chromatography.

### C. General Method 3c: Borane-THF

A solution of nitrile (1.0 eq) in THF was cooled in an ice/water bath before borane (1M in THF, 2.0 eq) was added dropwise. The reaction was allowed to warm to rt then heated to 60° C. for 16-96 h. MeOH was added and heating continued at 60° C. for 24 h before cooling to rt and concentrating. The product was isolated and purified using one of the following methods:
i) The crude product was loaded onto an SCX in MeOH and washed with MeOH. The product was eluted with 7M NH₃ in MeOH and the eluent concentrated.
ii) The crude product was purified by flash chromatography
iii) Boc₂O (1.2 eq) was added to the crude reaction mixture and stirred overnight. The solvent was evaporated in vacuo. The product was taken up in DCM, washed with water and brine, dried (Na₂SO₄), filtered and concentrated. The boc-protected amine was either used without further purification or purified by flash chromatography
   d. General Method 3d: NiCl₂

A solution of nitrile (1.0 eq), NiCl₂·6H₂O (1.0 eq) and Boc₂O (3.0 eq) in MeOH was cooled in an ice/water bath and sodium borohydride (NaBH₄) (5.0 eq) added portionwise. The reaction was allowed to warm to rt and stirred for 18 h. Water was added and the reaction mixture filtered, washed with THF and concentrated. The crude product was purified by flash chromatography.

e. General Method 3e: Hydrogenation; Pd/C

To a solution of nitrile (1.0 eq) in MeOH or EtOH under an inert atmosphere was added 10% Pd/C (0.1-0.2 eq). Additives such as HCl, sulfuric acid, or Boc₂O may optionally be added. The reaction was stirred under an atmosphere of H₂ (g) for 2-72 h. The catalyst was removed by filtration over Celite®, which was washed with EtOH. The product was isolated following concentration of the filtrate and used directly or purified by flash chromatography.

f. General Method 3f: Ring Saturation Reduction

A biaryl ring (1.0 eq) was dissolved in EtOH and subjected to hydrogenation in the H-Cube® at 70° C., 50 bar, 1 mL/min using a 10% Pd/C CatCart, recirculating when necessary. The solvent was removed in vacuo to afford the product which was used without purification.

### 4. General Method 4 (GM4): Buchwald

A suspension of amine or alcohol (1.0 eq), aryl halide (1.1 eq) and a base such as Cs₂CO₃, NaOtBu (2.0 eq) or LiHMDS (2.0 eq) in a degassed solvent such as THF or 1,4-dioxane was purged with N₂ (g). A Buchwald palladium precatalyst, such as BrettPhos Pd G3, (0.11 eq) was added and the mixture degassed and purged with N₂ (g) for 5 min. The reaction was heated in a sealed vial at rt−60° C. for 30 min-3 days as required. The product was isolated and purified using one of the following methods:
i) The reaction was quenched with AcOH (2.0 eq) and concentrated. The crude was purified by an SCX eluting with NH₃ in MeOH followed by purification by flash chromatography or prep HPLC.
ii) The reaction was quenched with AcOH (2.0 eq), filtered through Celite®, washing with EtOAc and the filtrate concentrated. The crude product was purified by flash chromatography
iii) The reaction mixture was acidified with AcOH (2.0 eq) and stirred for 5 min, 1M NH₃ in MeOH was added and the reaction mixture was concentrated on to silica and purified by flash chromatography.
iv) The reaction mixture was dry loaded on to silica and purified by flash chromatography.

### 5. General Method 5 (GM5): Boc Deprotection; HCl or TFA

a. General Method 5a: Boc Deprotection; HCl/Dioxane

A suspension of boc protected amine (1.0 eq) in 1,4-dioxane was treated with 4M HCl in dioxane (10.0 eq) was added and the reaction stirred at rt for 2-24 h. The product was isolated and purified using one of the following methods:
i) The reaction mixture was concentrated, optionally azeotroping with Et₂O or toluene to afford the product as a hydrochloride salt.
ii) The reaction mixture was concentrated and the product was converted to free base using a bicarbonate cartridge, PL-HCO₃ MP SPE (Agilent), loading in MeOH. The filtrate was concentrated and triturated with Et₂O to afford the product.
   b. General Method 5b: Boc Deprotection; TFA

A mixture of boc protected amine (1.0 eq) in DCM was treated with TFA (10.0 eq) and stirred at rt for 2 h. The mixture was passed directly through an SCX and washed with MeOH. The product was eluted with a solution of 7M NH₃ in MeOH and concentrated. The crude product was purified by flash chromatography or prep HPLC.

### 6. General Method 6 (GM6): Pyridone Chlorination

Pyridone (1.0 eq) was suspended in phosphorus oxychloride (large excess) and heated at reflux for 4 h. The reaction mixture was evaporated then azeotroped with toluene (×2). The residue was used immediately in the next step, taking care to exclude moisture.

### 7. General Method 7 (GM7): SNAr Alkylation (O and N)

a. General Method 7a (GM7a): SNAr O-Alkylation Using NaH

To a suspension of NaH (60% wt. on mineral oil) (1.04 eq) in DMF in an ice/water bath was added a solution of alcohol (1.02 eq) in DMF dropwise over 2 min. The mixture was allowed to warm to rt for 5 min before cooling again in an ice/water bath and treating with pyridyl halide or aryl halide (1.0 eq). The reaction mixture was maintained in an ice/water bath for 1 h then warmed to rt for 18 h. The reaction mixture was cooled in an ice/water bath and sat. Na₂CO₃ (aq) was added followed by water. This was extracted with EtOAc (×3) and the organic phases were combined, washed with 1:1 water/brine and brine. The organic phase was dried (MgSO₄), filtered and concentrated. The crude product was purified by flash chromatography.

b. General Method 7b (GM7b): SNAr O-Alkylation Using Cs₂CO₃

To a solution of alcohol (1.0 eq) and pyridyl halide (1.0 eq) in MeCN was added Cs₂CO₃ (2.0 eq) and the mixture was stirred in a sealed vial at 50° C. for 18-72 h. The product was isolated and purified using one of the following methods
i) The reaction mixture was cooled to rt and diluted with water (10 mL). The crude product was extracted into DCM, dried (MgSO₄), filtered and concentrated. The residue was purified by flash chromatography
ii) The reaction mixture was filtered through Celite® and the filtrate was concentrated to yield the crude product which was either used without further purification or purified by flash chromatography
iii) The crude reaction mixture was passed directly through an SCX in MeOH. The SCX was washed with MeOH and the product was eluted with 7M NH₃ in MeOH. The crude product was purified by flash chromatography.
   c. General Method 7c (GM7c): SNAr O-Alkylation Using NaOtBu

A solution of alcohol (1.0 eq), aryl bromide (1.0 eq) and NaOtBu (3.0 eq) in NMP was stirred in the microwave at 140° C. for 4 h. The crude reaction mixture was loaded onto an SCX in MeOH and washed with MeOH and the product was eluted with 7M NH₃ in MeOH (50 mL). The product was concentrated and purified by flash chromatography or prep HPLC.

d. General Method 7d (GM7d): SNAr N-Alkylation

Amine (1.0 eq) and halopyridine (1.0 eq) were dissolved in MeCN (3 mL). K₂CO₃ (3.0 eq) was added and the reaction was stirred at 60-120° C. for 60-90 min under thermal heating or microwave irradiation. The reaction was diluted with water and extracted with iso-propanol/CHCl3 (1:10) (×3). The combined organics were washed with brine, dried (MgSO₄) and concentrated. The product was isolated and used directly or purified by flash chromatography.

### 8. General Method 8 (GM8): 2,4-Dimethoxybenzyl Deprotection

A solution of 2,4-dimethyoxybenzyl protected amine (1.0 eq) in TFA (10 eq.) was stirred at rt-50° C. for 60 min. The reaction mixture was concentrated. The resulting residue was suspended in MeOH (2 mL) and loaded on to an SCX, which was flushed with MeOH (4×5 mL). The product was eluted with a solution of 1N NH₃ in MeOH (4×5 mL). The solvent was removed in vacuo. The crude product was either used without further purification or purified by flash chromatography or prep HPLC.

### 9. General Method 9 (GM9): Carbamate Protection

To a solution of aminopyridine (1.0 eq) and TEA (2.0 eq) in DCM (12 mL) in an ice/water bath was added methylchloroformate (3.0 eq) and the reaction was stirred at rt for 48 h. The reaction mixture was diluted with DCM and washed with water (20 mL). The aqueous was extracted with DCM (3×80 mL) and the combined organics dried (Na₂SO₄), filtered and concentrated. The crude product was triturated with EtOAc.

### 10. General Method 10 (GM10): Carbamate Deprotection

a. General Method 10a: KOH

A mixture of methyl carbamate (1 eq) and KOH (6 eq) in MeOH was stirred at 60° C. for 12-48 h. The product was isolated and purified using one of the following methods:
i) The reaction was quenched with AcOH (6.0 eq) and the mixture was left to stir for 5 min before being concentrated. The residue was passed directly through an SCX in MeOH. The SCX was washed with MeOH and the product was eluted with 7M NH₃ in MeOH and lyophilised.
ii) The reaction was quenched with AcOH (6.0 eq) and the mixture was left to stir for 5 min before being concentrated. The residue was passed directly through an SCX in MeOH. The SCX was washed with MeOH and the product was eluted with 7M NH₃ in MeOH. The product was purified by flash chromatography or prep HPLC
iii) The reaction was quenched with AcOH (6.0 eq), concentrated, and purified by prep HPLC.
   b. General Method 10b: LiOH

To a solution of methyl carbamate (1 eq) in THF/water (10:1) was added lithium hydroxide monohydrate (3-5 eq) and the reaction stirred at 60° C. for 18 h-4 days. The mixture was cooled to rt and concentrated. The crude residue was purified via flash chromatography or prep HPLC

### 11. General Method 11: SEM Deprotection

A mixture of TFA acid (10 eq) was added dropwise to a rapidly stirred solution of indole or azaindole (1.0 eq) in DCM. The mixture was stirred at rt for 18 h. The reaction mixture was passed directly through an SCX in MeOH. The SCX was washed with MeOH and the product was eluted with 2.5M NH₃ in MeOH and concentrated. The crude product was purified by flash chromatography or prep HPLC.

### 12. General Method 12: Alkylation and Cyclisation

To a solution of the amino substituted heteroaryl (e.g. pyridine) (1 eq) in EtOH (3 mL) were added NaHCO₃(2 eq) and the chloromethyl aldehyde or chloromethyl ketone as applicable (2 eq). The mixture was stirred for 20 h at 75° C. The mixture was then cooled to rt, filtered through Celite washing with EtOAc (50 mL) and concentrated. The product was purified by flash chromatography.

### 13. General Method 13: Hydroxylation

A solution of the aryl bromide or heteroaryl bromide (1.0 eq), bis(pinacolato)diboron (2.0 eq) and [1,1′-bis(diphenylphosphino)ferrocene]dichloropalladium(II) (0.1 eq) in anhydrous, degassed 1,4-dioxane was purged with N₂ (g) for 5 min. KOAc (3.0 eq) was added and the reaction stirred at 90° C. for 2-18 h. AcOH (2.0 eq) and water (1 mL) were added and the reaction stirred at rt for 15 min-18 h. A solution of H₂O₂ in water (30% w/w, 2 eq) was added and the reaction mixture stirred for 1-18 h.
i) Typically for basic compounds: solid Na₂S₂O₃ was added and the reaction was stirred at rt for 5 min then diluted with EtOAc, filtered through Celite and concentrated. The residue was purified by SCX and flash chromatography.
ii) Typically for non basic compounds: sat. Na₂S₂O₃ (aq) and sat. NaHCO₃ (aq) were added and the aqueous was extracted with EtOAc. The combined organics were washed with brine, dried (MgSO₄), concentrated, and purified by flash chromatography

### General Schemes

General Schemes 1-6 outline synthetic routes for certain example compounds and RgA, RgB, RgC and RgD refer to various substituents as required by the examples. For the sake of clarity General Schemes 1-6 are drawn with a bicyclopentyl central core group. Similar chemistry can be applied to examples with different core groups such as oxabicyclohexyl and oxabicycloheptyl.

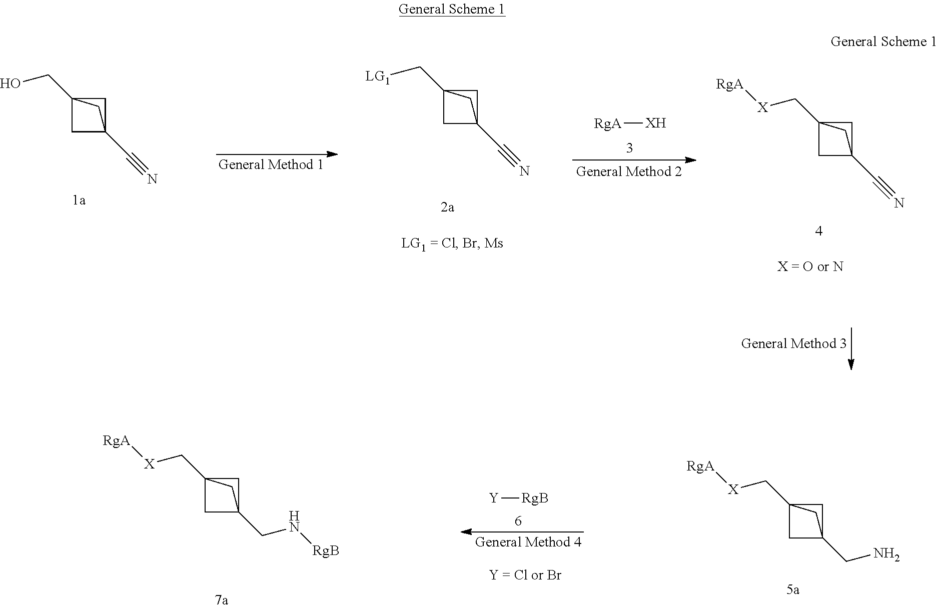

Alcohols such as 1a are typically reacted to form a suitable leaving group, such as halide or mesylate and can be generated using conditions well known in the art such as, for example; chlorination via a mesylate, bromination with PBr₃, or bromination with CBr₄ and PPh₃, using a suitable solvent such as DCM, THF or CCl₄ (General Method 1), to give compound 2a. Compound 2a is subsequently reacted with an alcohol or amine 3 under typical alkylation conditions (General Method 2, eg KOtBu or NaH in DMF or Cs₂CO₃ or K₂CO₃ in MeCN or DMSO, with heating as necessary). The nitrile 4 can be reduced to amine 5a under a variety of standard literature conditions well known in the art (General Method 3); for example under hydrogenation in the presence of Raney Ni, alternatively hydrogenation in the presence of Pd/C, or alternatively with NiCl₂ and sodium borohydride (NaBH₄) in the presence of Boc₂O, or alternatively with borane. The amine 5a is reacted with aryl bromide or chloride 6 under Buchwald coupling conditions (General Method 4). This Buchwald coupling is carried out for example using a Buchwald pre catalyst, such as BrettPhos Pd G3 or BrettPhos Pd G4, in the presence of a base such a sodium tert-butoxide, caesium carbonate, or lithium hexamethyldisilazide (LiHMDS), in a solvent such as 1,4-dioxane or THF. The aryl bromide or chloride 6 can be prepared from readily available starting materials using methods known in the art, or as described herein. Depending on the identity of RgB, a deprotection step (detailed above) may be required to obtain the example compound.

Alternatively, for example as shown in General Scheme 2, where material is available with the protected amine, for example compound 1b, a similar synthetic sequence can be applied.

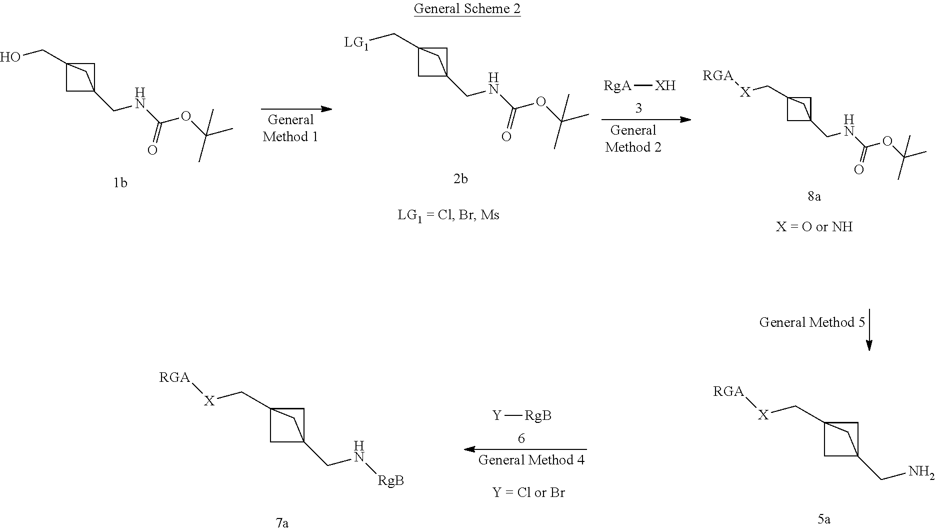

Following formation of a leaving group and alkylation, the tert-butoxy carbamate protecting group is removed from compound 8a using standard conditions such as TFA, or HCl in 1,4-dioxane (General Method 5). Finally, Buchwald coupling (General Method 4) completes the route.

The order of steps can also be reversed, for example as shown in General Scheme 3.

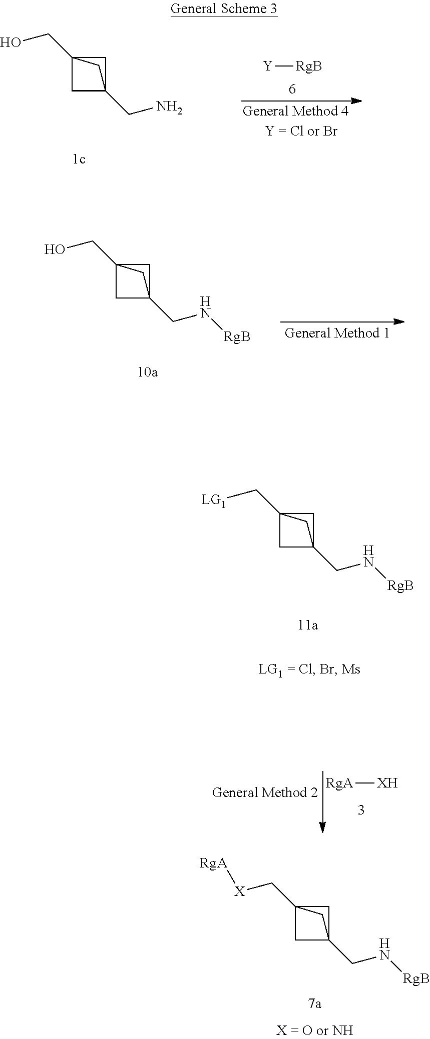

An amine such as 1c can undergo a Buchwald (General Method 4), followed by formation of a leaving group (General Method 1) and finally alkylation with compound 3 (General Method 2).

Furthermore the Buchwald coupling (General Method 4), is also possible with alcohols. This requires a suitable protecting group strategy as shown in General Schemes 4 and 5

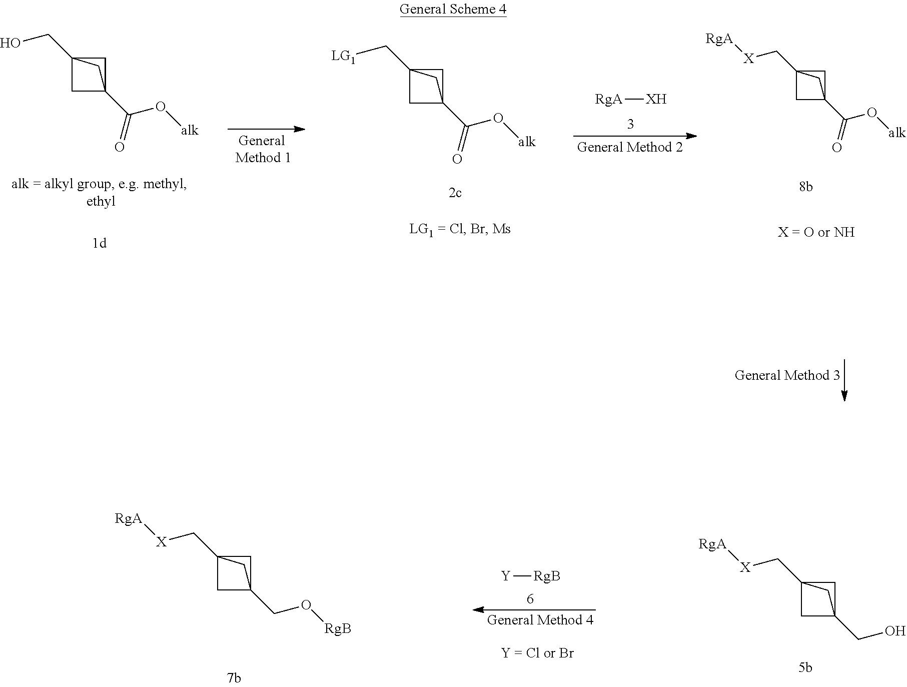

In General Scheme 4, a protected alcohol is in the form of an ester such as 1d. Following the usual sequence of steps, formation of a leaving group and alkylation, the ester 8b is reduced using standard conditions such as LiAlH₄ (General Method 3) to reveal the alcohol. Finally, Buchwald coupling (General Method 4) of alcohol 5b completes the route. This typically requires an elevated temperature, for example 90° C. or above and in some cases alternative catalysts, such as RockPhos Pd G3 are more suitable.

Again, the order of steps can also be reversed, for example as shown in General Scheme 5.

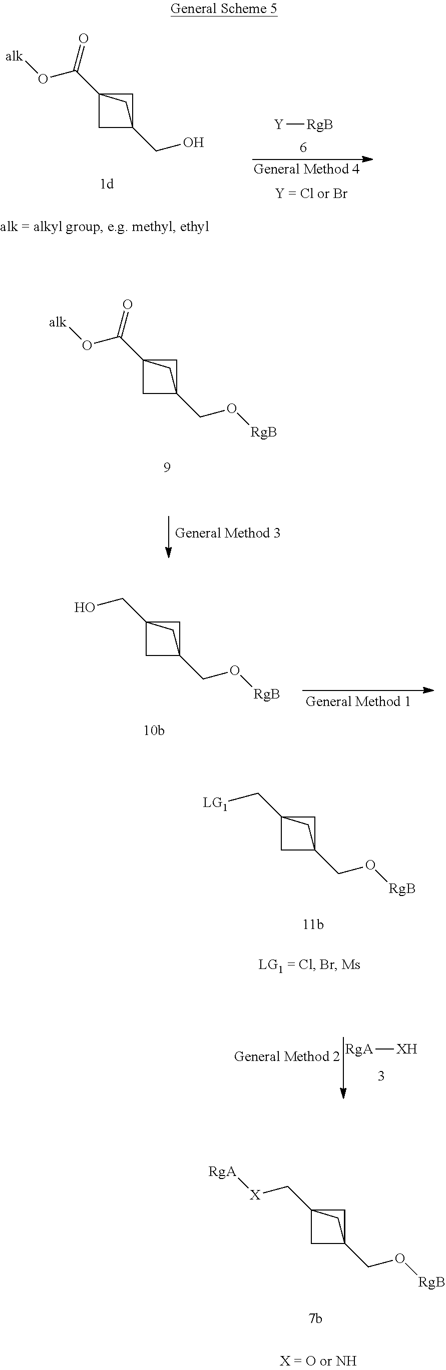

The alcohol such as 1d can undergo a Buchwald (General Method 4), followed by reduction of the ester, formation of a leaving group (General Method 1) and finally alkylation with compound 3 (General Method 2).

In examples, where RgA contains a bicyclic aromatic, it can be partially saturated during the synthesis, as shown in General Scheme 6.

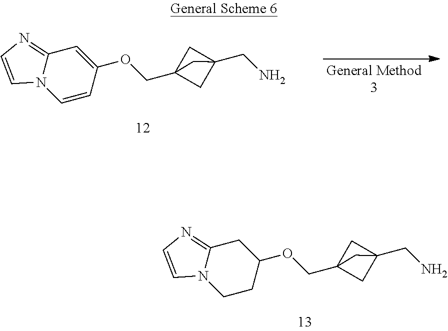 [SMILES: NCC12CC(COc3ccn4ccnc4c3)(C1)C2 ; NCC12CC(COC3CCn4ccnc4C3)(C1)C2]

Typically the aromatic ring such as 12 is subjected to hydrogenation at elevated temperature (General Method 3) using H₂, 10% Pd/C to give compound 13. This transformation can take place on the free or protected amine.

In example compounds described herein containing a primary or secondary amine, a protecting group strategy may be required. Alternative protecting groups can be used with different deprotection conditions, that is, an orthogonal protecting group strategy can be applied. General Schemes 7-12 outline possible protecting group strategies that may be used for the synthesis of the examples.

For example, for compounds defined herein containing a 6,6 ring system, as shown in General Scheme 7, a protected amine can be installed. Initially the pyridone 14 may be chlorinated, typically using phosphorous oxychloride (General method 6). The resulting chloride 15 may be reacted with 2,4-dimethoxybenzylamine using General Method 7, for example using basic conditions such as potassium carbonate or pyridine in a solvent such as NMP or MeCN, either thermally and/or under microwave conditions. RgT refers to various substituents as required by the example compounds.

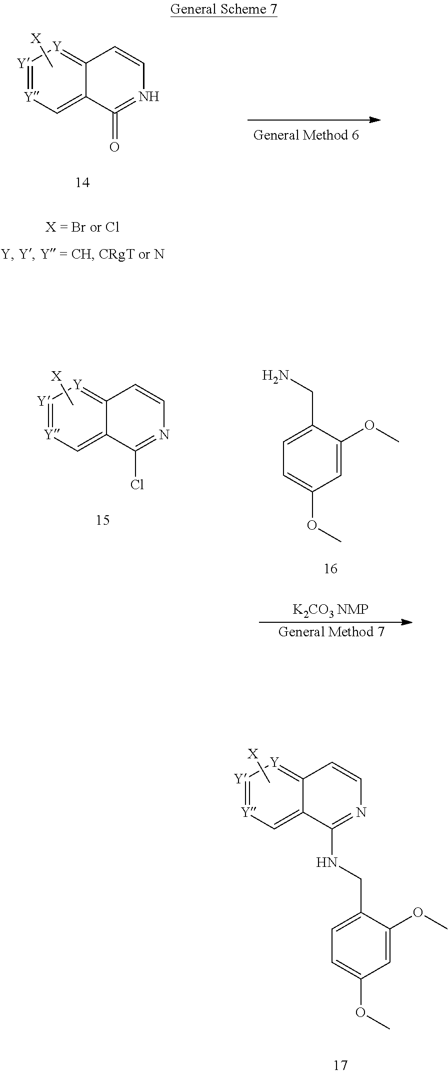

Typically, at the end of the synthetic sequence, the 2,4-dimethoxybenzyl protecting group is removed using undiluted TFA at 50° C. (General Scheme 8, General Method 8). RgT, RjA and RjB refer to various substituents as required by the examples.

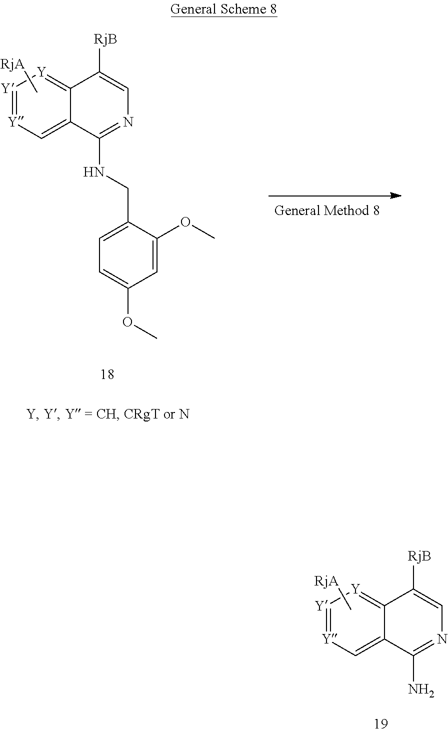

Alternatively, when starting materials are available with the amine already installed, a carbamate protecting group can be used. For example, as outlined in General Scheme 9, the amine is reacted with methyl chloroformate under basic conditions with organic bases such as TEA or DIPEA in a solvent such as DCM to afford the methyl carbamate 21 (General Method 9). RjC refers to various substituents as required by the examples.

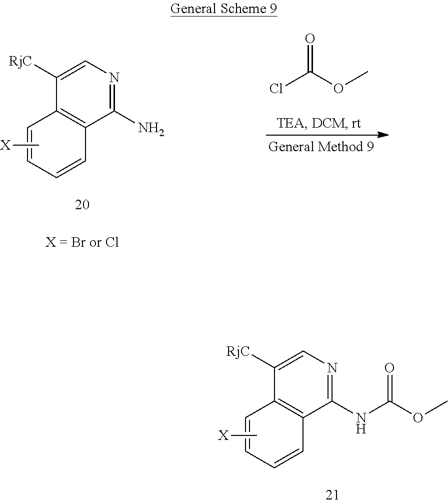

Typically at the end of the synthetic sequence the methyl carbamate protecting group is deprotected using basic conditions, such as KOH or LiOH in solvents such as 1,4-dioxane, MeCN, THF and optionally 10% water, at elevated temperature, typically 50° C. (General Scheme 10, General Method 10). RjC and RjD refer to various substituents as required by the examples.

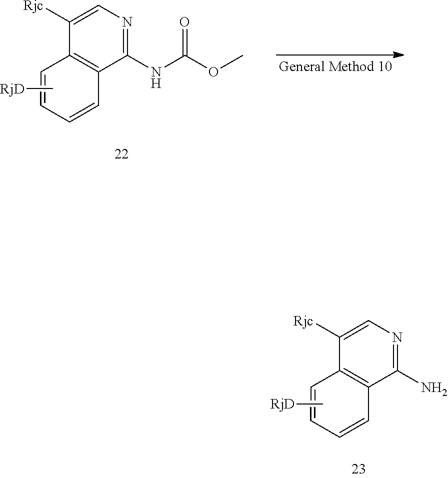

### General Scheme 10

Another protecting group that may be used where example compounds described herein contain a 6,6 ring system is boc.

Where, for example, example compounds described herein contain a 5,6 ring system, SEM, boc and sulfonyl protecting groups may typically be used. Protecting groups may subsequently be deprotected using standard literature procedures, for example those described by T. W. Greene and P. G. M. Wuts in “Protective groups in organic chemistry” John Wiley and Sons, 4th Edition, 2006.

An example of the installation of a SEM protecting group is shown in General Scheme 11 whereby the indole 24 is treated with a base such as NaH in a solvent such as DMF, followed by addition of 2-(trimethylsilyl)ethoxymethyl chloride (General Method 2).

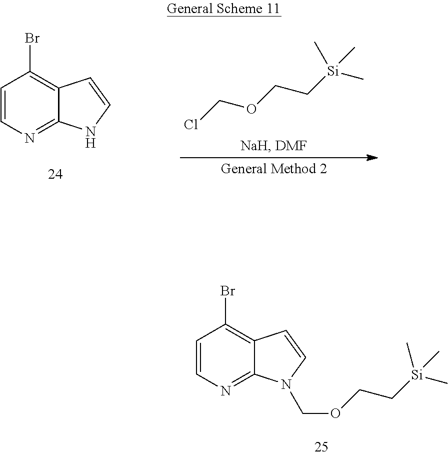

Typically at the end of the synthetic sequence the SEM protecting group is deprotected using acidic conditions such as TFA in DCM (General Scheme 12, General Method 11). RjD refers to various substituents as required by the examples.

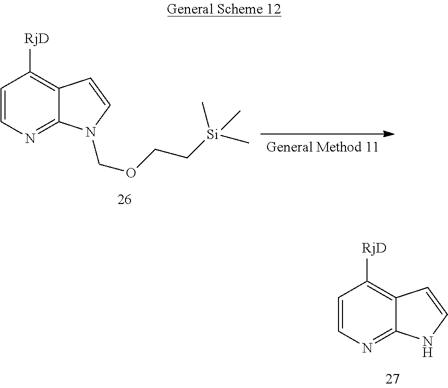

In examples where RgA contains an aromatic or heteroaromatic bicycle they can be synthesised, for example, as depicted in General Scheme 13.

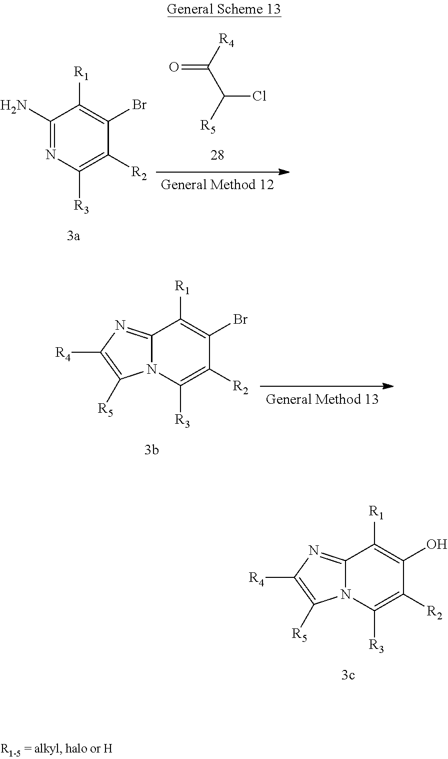

Amino pyridines such as 3a can be reacted with chloromethyl aldehydes and ketones 28 by refluxing in a solvent such as ethanol (General Method 12). Hydroxylation of the aryl bromide 3b to the corresponding aryl alcohol 3c is typically completed in a one-pot tandem process via Miyaura borylation (Palladium(0)-Catalyzed Cross-Coupling Reaction of Alkoxydiboron with Haloarenes: A Direct Procedure for Arylboronic Esters. T. Ishiyama, M. Murata, N. Miyaura, J. Org. Chem., 1995, 60, 7508-7510) and subsequent hydroxylation using hydrogen peroxide (General Method 13).

Other examples herein may be based on an azabicyclohexyl central core group, as shown in General Schemes 14-17. These General Schemes may also be applied to other central core groups such as bicyclopentyl, oxabicyclohexyl and oxabicycloheptyl. The starting materials may be readily available from commercial sources or may be known in the literature. Alternatively, some prior manipulation may be required, for example, as shown in Scheme 14, a carboxylic acid such as 1e can be esterfied using methods generally known in the art, for example via formation of the acid chloride in an appropriate alcohol solvent such as methanol.

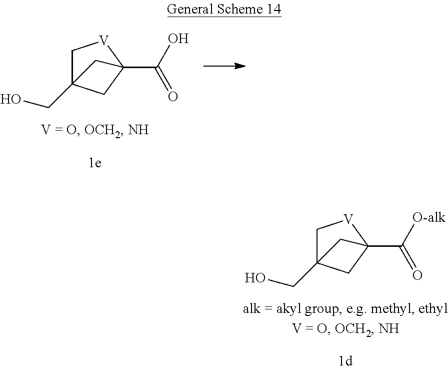

When azabicyclohexyl rings are used they may be protected with a suitable nitrogen protecting group such as Boc or Cbz. Alternatively the nitrogen can be reacted using methods generally known in the art to provide amide, urea, alkyl and sulfonamide analogues as shown in General Scheme 15.

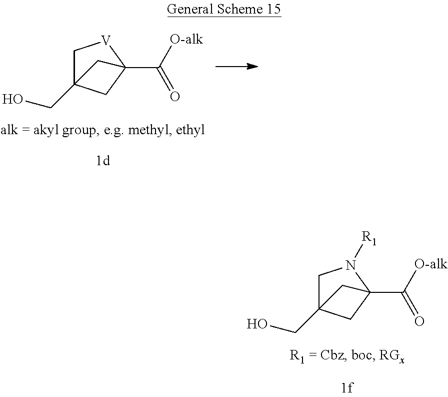

As outlined in previous schemes and as repeated in General Scheme 16, RgA can be installed by transforming the alcohol to a leaving group, such as a mesylate or halide and reacting with aryl alcohols. Alternatively the alcohol can be directly reacted with phenols under Mitsunobu conditions. The alcohol can also be coupled with aryl halides under SNAr in the presence of NaH (General Method 7a), or under Buchwald conditions (General Method 4), typically using RockPhos Pd G3 as the catalyst and elevating the temperature to 100° C. The ester 8b is then reduced to alcohol 5c under conditions well known in the art such as lithium borohydride (LiBH₄) or LiAlH₄ depending on other functionality contained in the compound. Oxidation to aldehyde 5d is typically achieved using a reagent such as Dess Martin periodinane. Finally, reductive amination with amine 6a completes the route. Alternatively (not shown in the Schemes) the alcohol 5c can be transformed to an amine using methods generally known in the art, such as via conversion to a leaving group (General Method 1) then via an azide or Gabriel synthesis to provide the primary amine and finally completing the route via a Buchwald coupling (General Method 4) as shown previously in General Scheme 1.

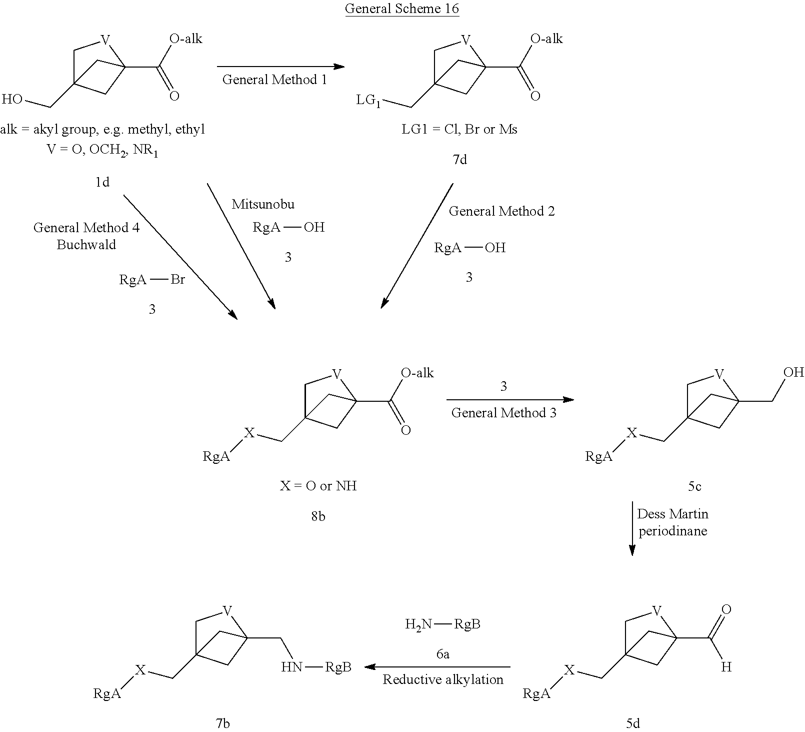

When azabicyclohexyl rings are used, in which the nitrogen is protected by a protecting group (i.e. 7b where V=NR₁), a late stage functionalisation is possible, as shown in General Scheme 17. The nitrogen protecting group is removed using the appropriate conditions (for example boc deprotection via General Method 5, or 2,4-dimethoxybenzyl deprotection via General Method 9, or carbamate deprotection via General Method 10, or SEM deprerotection via General Merthod 11). Manipulation of 7c using chemistry well known in the art can provide a variety of compound classes. For example, reaction of 7c with carboxylic acids or acid chlorides can be used to provide amides or reaction with sulphonyl chlorides can provide suphonamides. Reductive alkylation with aldehydes can give alkylated compounds. Buchwald reactions (General Method 4) or SNAr alkylation (General Method 7) with aryl halides can install aromatic groups. Reaction with CDI and alcohols can provide carbamates and with amines can provide ureas.

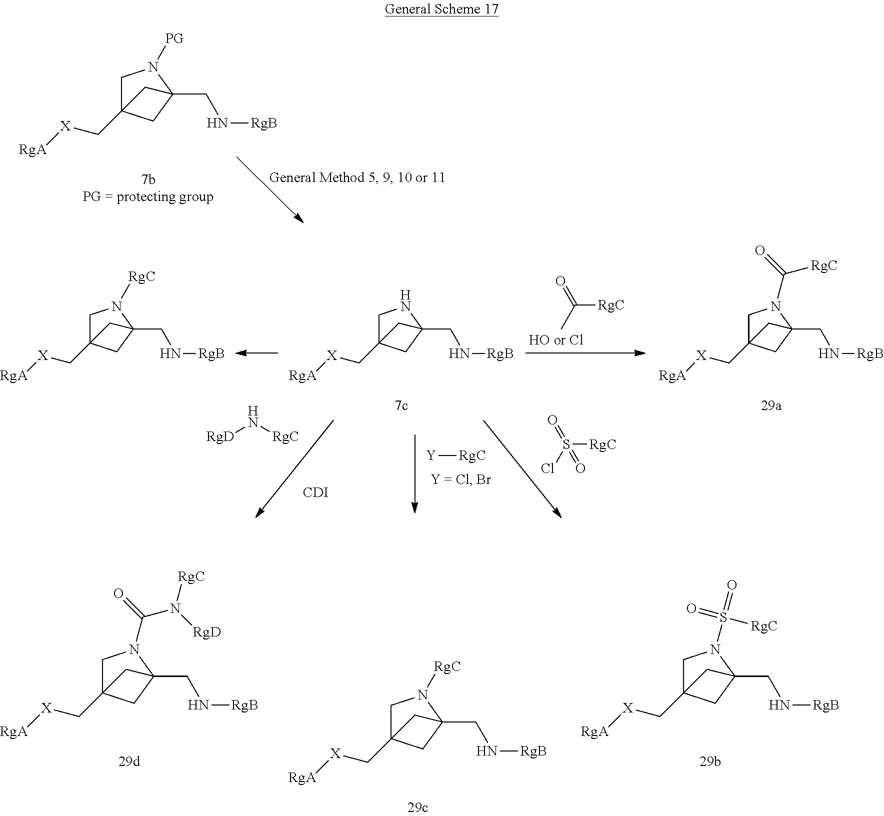

### Synthesis of Intermediates

### Intermediate 1

### Methyl (6-bromoisoquinolin-1-yl)carbamate

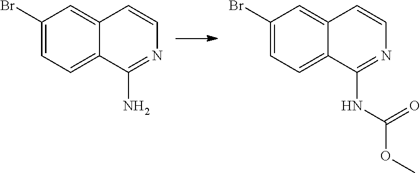 [SMILES: Nc1nccc2cc(Br)ccc12 ; COC(=O)Nc1nccc2cc(Br)ccc12]

Following General Method 9, 6-bromoisoquinolin-1-amine (1.50 g, 6.72 mmol) was protected. The crude was suspended in water (100 mL) and stirred for 30 min before being collected by filtration and dried in the vacuum oven overnight to give the product (1.12 g, 44% yield).

[M+H]⁺=281.1

¹H NMR (500 MHz, DMSO-d6) 3.70 (3H, s), 7.58-7.72 (1H, m), 7.79 (1H, d, J=9.0, 2.0 Hz), 8.04 (1H, d, J=9.1 Hz), 8.25-8.30 (1H, m), 8.33 (1H, d, J=5.8 Hz), 10.18 (1H, s)

### Intermediate 2

### Methyl (6-bromo-4-chloroisoquinolin-1-yl)carbamate

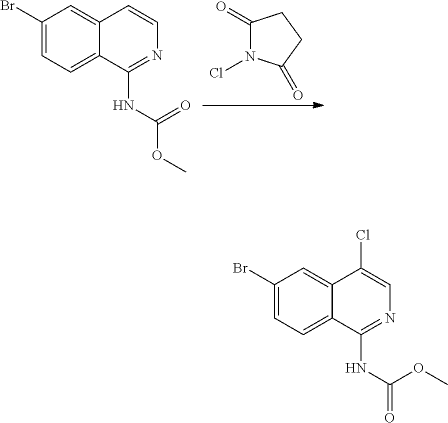 [SMILES: COC(=O)Nc1nccc2cc(Br)ccc12 ; O=C1CCC(=O)N1Cl ; COC(=O)Nc1ncc(Cl)c2cc(Br)ccc12]

Methyl N-(6-bromo-1-isoquinolyl)carbamate (100 mg, 0.36 mmol) was dissolved in chloroform (5 mL), NCS (52 mg, 0.39 mmol) was added and the reaction stirred at reflux for 18 h. To the reaction was added sat. NaHCO₃ (aq.) (30 mL) and it was washed with DCM (30 mL), dried (Na₂SO₄) and concentrated. The crude product was purified by flash chromatography (Silica, 0-80% EtOAc in Pet. Ether) to give the product (74 mg, 59% yield).

[M+H]⁺=316.8/318.7

¹H NMR (CDCl₃, 400 MHz) δ 3.84 (3H, s), 7.36 (1H, s), 7.75 (1H, dd, J=9.0, 1.9 Hz), 7.93 (1H, d, J=9.0 Hz), 8.37 (2H, d, J=4.9 Hz)

### Intermediate 3

### Methyl (5-bromoisoquinolin-1-yl)carbamate

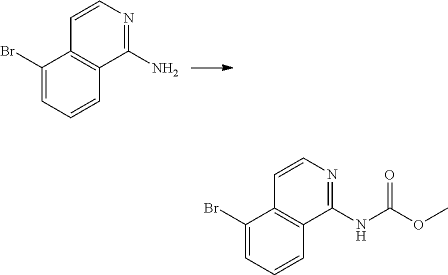 [SMILES: Nc1nccc2c(Br)cccc12 ; COC(=O)Nc1nccc2c(Br)cccc12]

Following General Method 9, 5-bromoisoquinolin-1-amine (1.12 g, 5.02 mmol) was protected. The product was dried under high vacuum to yield (838 mg, 56% yield)

[M+H]₊=281.1

### Intermediate 4

### 5-Bromo-N-(2,4-dimethoxybenzyl)isoquinolin-1-amine

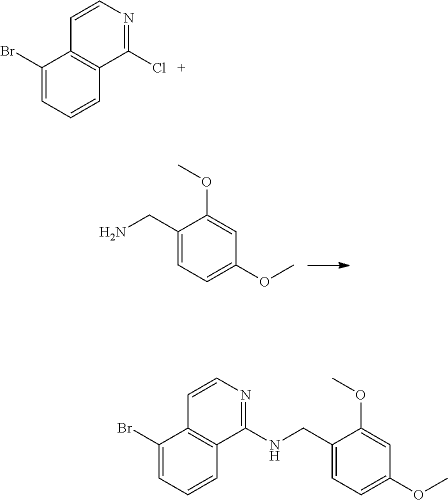 [SMILES: Clc1nccc2c(Br)cccc12 ; COc1ccc(CN)c(OC)c1 ; COc1ccc(CNc2nccc3c(Br)cccc23)c(OC)c1]

To a solution of 5-bromo-1-chloroisoquinoline (0.5 g, 2.06 mmol) in pyridine (3 mL), was added 2,4-dimethoxybenzylamine (0.69 g, 4.12 mmol). The reaction was heated at 150° C. in a CEM Microwave for 60 min. The mixture was diluted with DCM (20 mL) and water (20 mL). The aqueous layer was re-extracted with DCM (3×10 mL) and the combined organics were washed with brine (20 mL). The organic layer was dried (Na₂SO₄), filtered and concentrated to afford the crude product. Purification was performed by flash chromatography (Silica, 20-50% EtOAc in Pet. Ether) to afford the product (276 mg, 50% yield).

[M+H]⁺=373.0/375.0

¹H NMR (DMSO-d₆, 400 MHz) δ 3.71 (3H, d, J=2.6 Hz), 3.82 (3H, d, J=2.8 Hz), 4.62 (2H, d, J=5.4 Hz), 6.41 (1H, dd, J=8.5, 2.5 Hz), 6.56 (1H, d, J=2.6 Hz), 6.94-7.14 (2H, m), 7.42 (1H, t, J=8.0 Hz), 7.96 (3H, ddd, J=16.4, 7.1, 3.2 Hz), 8.38 (1H, d, J=8.2 Hz)

### Intermediate 4a

### N1-(2,4-Dimethoxybenzyl)isoquinoline-1,5-diamine

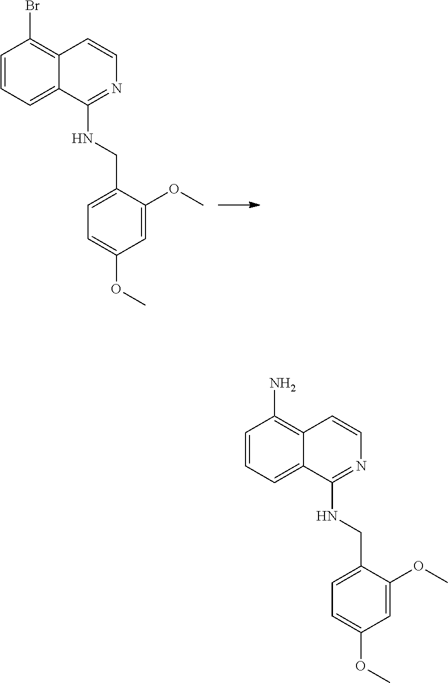 [SMILES: COc1ccc(CNc2nccc3c(Br)cccc23)c(OC)c1 ; COc1ccc(CNc2nccc3c(N)cccc23)c(OC)c1]

A mixture of 5-bromo-N-[(2,4-dimethoxyphenyl)methyl]isoquinolin-1-amine (746 mg, 2.0 mmol), 2,2,2-trifluoroacetamide (339 mg, 3.0 mmol), CuI₂ (38 mg, 0.2 mmol), K₂CO₃ (553 mg, 4.0 mmol) and N,N′-Dimethylethylenediamine (35 mg, 0.4 mmol) were combined in a reaction vial. Anhydrous 1,4-Dioxane (7 mL) was added, and the suspension purged with N₂, before being capped and then heated to 75° C. for 24 h. The reaction was recharged with 2,2,2-trifluoroacetamide (339 mg, 3.0 mmol), CuI₂ (38 mg, 0.2 mmol), K₂CO₃ (553 mg, 4.0 mmol) and N,N′-Dimethylethylenediamine (35 mg, 0.4 mmol). The mixture was degassed with N₂ and heated at 70° C. for 18 h. Water was added (3 ml) and the reaction heated at 80° C. for 6 h. The mixture was partitioned between EtOAc (30 mL) and water (10 mL). The aqueous layer was extracted with further EtOAc (2×30 mL) and the combined organics were washed with brine (50 mL), dried (Na₂SO₄), filtered and concentrated. The product was purified by flash chromatography (Silica, 0-100% EtOAc/DCM) to afford the product (492 mg, 72% yield).

[M+H]⁺ 310.1

¹H NMR (CDCl₃, 400 MHz) δ 3.79 (3H, s), 3.85 (3H, s), 4.10 (2H, br s), 4.72 (2H, d, J=5.3 Hz), 5.58-5.67 (1H, m), 6.44 (1H, dd, J=8.2, 2.4 Hz), 6.49 (1H, d, J=2.4 Hz), 6.78-6.86 (2H, m), 7.12 (1H, dt, J=8.4, 1.1 Hz), 7.21 (1H, dd, J=8.4, 7.4 Hz), 7.30 (1H, d, J=8.2 Hz), 8.01 (1H, d, J=6.1 Hz)

### Intermediate 5

### 4-Bromo-1-((2-(trimethylsilyl)ethoxy)methyl)-1H-pyrrolo[2,3-b]pyridine

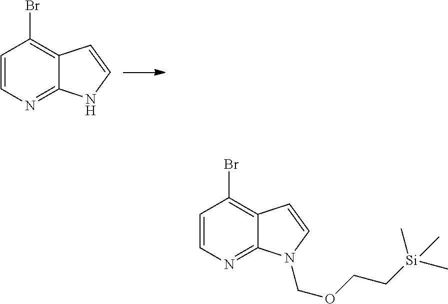 [SMILES: Brc1ccnc2[nH]ccc12 ; C[Si](C)(C)CCOCn1ccc2c(Br)ccnc21]

Following General Method 2a, 4-bromo-1H-pyrrolo[2,3-b]pyridine (500 mg, 2.54 mmol) was reacted with (2-(chloromethoxy)ethyl)trimethylsilane (494 μL, 2.79 mmol) for 2 h. The reaction mixture was quenched by the careful addition of water (10 mL) followed by repeat extraction with EtOAc (3×20 mL). The combined organic layers were then washed with saturated NaHCO₃ (30 mL), water (30 mL) and brine (30 mL), before being dried (MgSO₄), filtered and concentrated. The crude product was purified by flash chromatography (Silica, 0-100% EtOAc in iso-hexane) to afford the product (500 mg, 57% yield) as a clear colourless oil

[M+H]⁺=327.2

¹H NMR (500 MHz, DMSO-d6) δ −0.11 (s, 9H), 0.75-0.86 (m, 2H), 3.43-3.56 (m, 2H), 5.63 (s, 2H), 6.52 (d, J=3.6 Hz, 1H), 7.43 (d, J=5.1 Hz, 1H), 7.79 (d, J=3.6 Hz, 1H), 8.16 (d, J=5.1 Hz, 1H)

### Intermediate 6

### 5-Bromo-3-chloro-1-((2-(trimethylsilyl)ethoxy)methyl)-1H-pyrrolo[2,3-b]pyridine

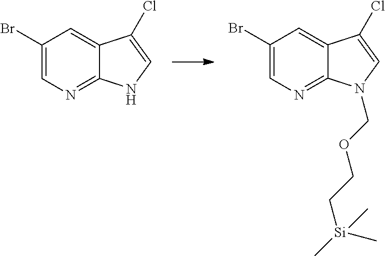 [SMILES: Clc1c[nH]c2ncc(Br)cc12 ; C[Si](C)(C)CCOCn1cc(Cl)c2cc(Br)cnc21]

Following General Method 2a, 5-bromo-3-chloro-1H-pyrrolo[2,3-b]pyridine (480 mg, 2.07 mmol) was reacted (2-(chloromethoxy)ethyl)trimethylsilane (0.4 mL, 2.28 mmol) for 2 h. The reaction was quenched with water (2 mL) and diluted with EtOAc (40 mL). The organic layer was washed with water (20 mL), 1M HCl (aq) (20 mL), 1:1 water/brine (20 mL) and brine (20 mL), dried (MgSO₄), filtered and concentrated. The crude product was purified by flash chromatography (Silica, 0-100% EtOAc in iso-hexane) to afford the product (485 mg, 60% yield).

[M+H]⁺=363.0

¹H NMR (500 MHz, DMSO-d6) δ −0.10 (9H, s), 0.81 (2H, t, J=7.9 Hz), 3.51 (2H, t, J=7.9 Hz), 5.60 (2H, s), 7.98-8.01 (1H, m), 8.20-8.24 (1H, m), 8.44-8.47 (1H, m)

### Intermediate 7

### 6-Bromo-N-(2,4-dimethoxybenzyl)-4-fluoroisoquinolin-1-amine

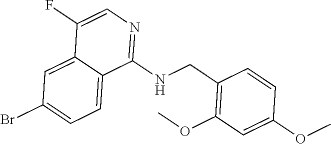 [SMILES: COc1ccc(CNc2ncc(F)c3cc(Br)ccc23)c(OC)c1]

### 6-Bromo-1-chloro-4-fluoroisoquinoline

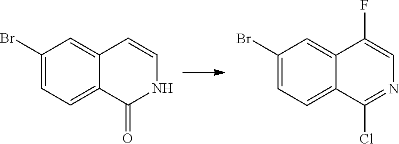 [SMILES: O=c1[nH]ccc2cc(Br)ccc12 ; Fc1cnc(Cl)c2ccc(Br)cc12]

A solution of 6-bromo-2H-isoquinolin-1-one (8.0 g, 35.7 mmol) and Selectfluor (15.2 g, 42.8 mmol) in MeCN (100 mL) and MeOH (100 mL) was heated at 50° C. for 60 min. The reaction mixture was evaporated and reacted using General Method 6, in 1,2-dichloroethane (200 mL) with benzyltriethylammonium chloride (820 mg, 3.6 mmol) and phosphorus oxychloride (50 mL). The reaction mixture was evaporated and the residue partitioned between DCM (500 mL) and water (500 mL). The organic layer was washed with water (300 mL), brine (300 mL), dried (MgSO₄) and evaporated. The crude was purified by flash chromatography (Silica, 5% EtOAc in Pet. Ether) to give the product (6.88 g, 74% yield).

[M+H]⁺=260.0

¹H NMR (500 MHz, CDCl₃) δ 8.27 (d, J=1.9 Hz, 1H), 8.21-8.16 (m, 2H), 7.84 (dd, J=9.1, 1.9 Hz, 1H)

¹⁹F NMR (471 MHz, CDCl₃) δ −139.8 (s)

### 6-Bromo-N-(2,4-dimethoxybenzyl)-4-fluoroisoquinolin-1-amine

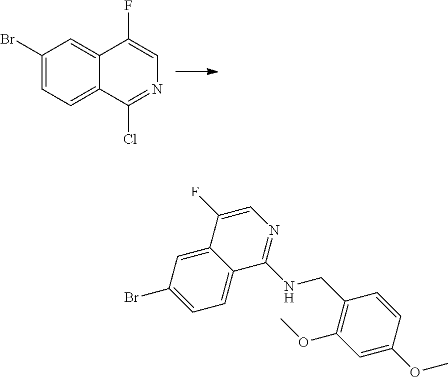 [SMILES: Fc1cnc(Cl)c2ccc(Br)cc12 ; COc1ccc(CNc2ncc(F)c3cc(Br)ccc23)c(OC)c1]

Following General Method 7d, 6-bromo-1-chloro-4-fluoroisoquinoline (6.88 g, 26.4 mmol) was reacted with 2,4-dimethoxybenzylamine (5.95 mL, 39.6 mmol) in 1-methyl-2-pyrrolidinone (100 mL) at 100° C. for 48 h. The crude product was purified by flash chromatography (Silica, 0-20% EtOAc in Pet. Ether) to give the product (3.2 g, 31% yield).

[M−H]⁻=389.2

¹H NMR (500 MHz, DMSO) δ 8.35 (dd, J=9.0, 2.2 Hz, 1H), 7.98 (d, J=2.0 Hz, 1H), 7.90-7.70 (m, 3H), 7.07 (d, J=8.3 Hz, 1H), 6.55 (d, J=2.4 Hz, 1H), 6.41 (dd, J=8.5, 2.4 Hz, 1H), 4.56 (d, J=5.5 Hz, 2H), 3.82 (s, 3H), 3.72 (s, 3H)

¹⁹F NMR (471 MHz, DMSO) δ −157.4 (s)

### Intermediate 8

### 5-Bromo-N-(2,4-dimethoxybenzyl)-4-fluoroisoquinolin-1-amine

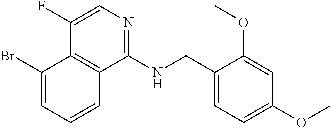 [SMILES: COc1ccc(CNc2ncc(F)c3c(Br)cccc23)c(OC)c1]

### 5-Bromo-1-chloro-4-fluoroisoquinoline

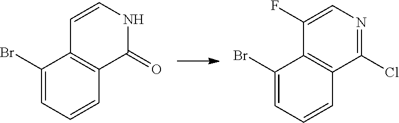 [SMILES: O=c1[nH]ccc2c(Br)cccc12 ; Fc1cnc(Cl)c2cccc(Br)c12]

A solution of 5-bromo-2H-isoquinolin-1-one (9.0 g, 40.2 mmol) and Selectfluor (17.1 g, 48.2 mmol) in MeCN (120 mL) and MeOH (120 mL) were heated at 50° C. for 3 h. The reaction mixture was evaporated and reacted using General Method 6, in 1,2-dichloroethane (200 mL) using benzyltriethylammonium chloride (915 mg, 4.0 mmol) and phosphorus oxychloride (45 mL) at 90° C. for 24 h. The reaction mixture was evaporated and the residue partitioned between DCM (500 mL) and water (500 mL). The organic layer was washed with water (300 mL) and brine (300 mL), dried (MgSO₄) and evaporated. The crude was purified by flash chromatography (Silica, 0-30% EtOAc in Pet. Ether) to give the product (5.70 g, 55% yield).

[M+H]⁺_{=261.9}

¹H NMR (500 MHz, CDCl3) δ 8.39-8.33 (m, 1H), 8.23 (d, J=4.0 Hz, 1H), 8.12-8.06 (m, 1H), 7.57 (t, J=8.0 Hz, 1H)

### 5-Bromo-N-(2,4-dimethoxybenzyl)-4-fluoroisoquinolin-1-amine

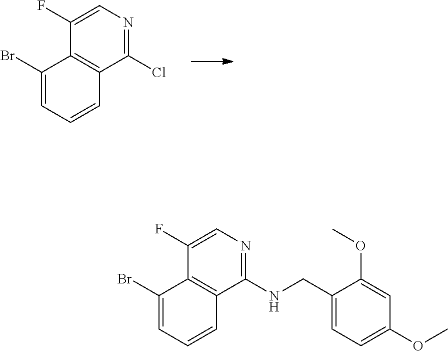 [SMILES: Fc1cnc(Cl)c2cccc(Br)c12 ; COc1ccc(CNc2ncc(F)c3c(Br)cccc23)c(OC)c1]

Following General Method 7d, 5-bromo-1-chloro-4-fluoroisoquinoline (5.70 g, 21.9 mmol) was reacted with 2,4-dimethoxybenzylamine (4.93 mL, 32.8 mmol) in 1-methyl-2-pyrrolidinone (80 mL) at 100° C. for 48 h. The crude product was purified by flash chromatography (Silica, 0-30% EtOAc in Pet. Ether) to give the product (1.05 g, 12% yield).

¹H NMR (500 MHz, DMSO) δ 8.43 (dd, J=8.1, 2.3 Hz, 1H), 8.06 (dd, J=7.6, 0.9 Hz, 1H), 7.89 (d, J=5.1 Hz, 1H), 7.81 (t, J=5.6 Hz, 1H), 7.49 (t, J=8.0 Hz, 1H), 7.05 (d, J=8.3 Hz, 1H), 6.56 (d, J=2.4 Hz, 1H), 6.41 (dd, J=8.4, 2.4 Hz, 1H), 4.57 (d, J=5.5 Hz, 2H), 3.82 (s, 3H), 3.72 (s, 3H)

¹⁹F NMR (471 MHz, DMSO) δ −149.9(s)

[M−H]⁻=389.2

### Intermediate 9

### [3-(Imidazo[1,2-a]pyridin-7-yloxymethyl)-1-bicyclo[1.1.1]pentanyl]methanamine

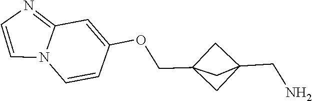 [SMILES: NCC12CC(COc3ccn4ccnc4c3)(C1)C2]

### (3-Cyano-1-bicyclo[1.1.1]pentanyl)methyl methanesulfonate

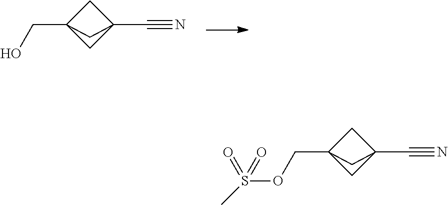

Following general method 1a, 3-(hydroxymethyl)bicyclo[1.1.1]pentane-1-carbonitrile (320 mg, 2.6 mmol) was reacted with methane sulfonyl chloride (241 μL, 3.12 mmol) and TEA (507 μL, 3.64 mmol). The mixture was stirred while cooling for 90 min, after which time water (30 mL) was added and the mixture extracted with DCM (2×50 mL), dried (MgSO₄), filtered and concentrated to give the product (597 mg, 100% yield).

¹H NMR (400 MHz, CDCl₃) δ 2.30 (s, 6H), 3.03 (s, 3H), 4.19 (s, 2H)

### 3-(Imidazo[1,2-a]pyridin-7-yloxymethyl)bicyclo[1.1.1]pentane-1-carbonitrile

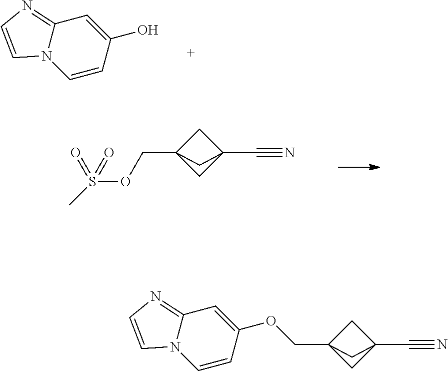 [SMILES: Oc1ccn2ccnc2c1 ; CS(=O)(=O)OCC12CC(C#N)(C1)C2 ; N#CC12CC(COc3ccn4ccnc4c3)(C1)C2]

Following General Method 2a, imidazo[1,2-a]pyridin-7-ol hydrochloride (445 mg, 2.61 mmol) was reacted with (3-cyano-1-bicyclo[1.1.1]pentanyl)methyl methanesulfonate (597 mg, 2.61 mmol) in the presence of TEA (364 μL, 2.61 mmol) for 7 days at rt. Water (30 mL) was added and the mixture extracted with EtOAc (2×80 mL). The combined organics were washed with brine (50 mL), dried (MgSO₄) and concentrated. The residue was taken up in Et₂O (5 mL) and resultant slurry was sonicated and filtered and the solid was dried in vacuo to afford the product (320 mg, 1.2 mmol, 46% yield).

[M+H]⁺=240.1

¹H NMR (CDCl₃, 400 MHz) δ 2.34 (6H, s), 3.98 (2H, s), 6.50 (1H, dd, J=7.4, 2.5 Hz), 6.75-6.79 (1H, m), 7.42 (1H, dd, J=1.4, 0.7 Hz), 7.48 (1H, d, J=1.4 Hz), 7.92-7.97 (1H, m)

### [3-(Imidazo[1,2-a]pyridin-7-yloxymethyl)-1-bicyclo[1.1.1]pentanyl]methanamine

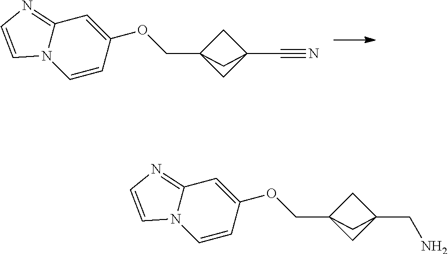 [SMILES: N#CC12CC(COc3ccn4ccnc4c3)(C1)C2 ; NCC12CC(COc3ccn4ccnc4c3)(C1)C2]

The nitrile, 3-(imidazo[1,2-a]pyridin-7-yloxymethyl)bicyclo[1.1.1]pentane-1-carbonitrile (320 mg, 1.2 mmol) was reduced following General Method 3a using a Raney Ni CatCart. The solvent was removed in vacuo to afford the product (315 mg, 97% yield).

[M+H]⁺=244.1

¹H NMR (CDCl₃, 400 MHz) δ 1.72 (6H, s), 2.76 (2H, s), 4.01 (2H, s), 6.53 (1H, dd, J=7.4, 2.5 Hz), 6.83 (1H, d, J=2.5 Hz), 7.37-7.41 (1H, m), 7.47 (1H, d, J=1.3 Hz), 7.92 (1H, dd, J=7.4, 0.8 Hz), NH₂ not seen

### Intermediate 10

### Tert-butyl N-[[3-(chloromethyl)-1-bicyclo[1.1.1]pentanyl]methyl]carbamate

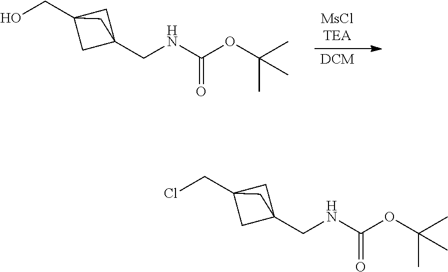 [SMILES: CC(C)(C)OC(=O)NCC12CC(CO)(C1)C2 ; CC(C)(C)OC(=O)NCC12CC(CCl)(C1)C2]

Following General Method 1b, tert-butyl N-{[3-(hydroxymethyl)bicyclo[1.1.1]pentan-1-yl]methyl}carbamate (170 mg, 0.75 mmol) was reacted with triethylamine (188 μL, 1.35 mmol) and methane sulfonyl chloride (87 μL, 1.12 mmol) at rt for 20 h. The reaction mixture was diluted with sat. NaHCO₃ (aq) and the aqueous extracted with DCM (3×30 mL). The combined organics were washed with brine (25 mL), dried (MgSO₄), filtered and concentrated in vacuo to afford the product (290 mg, 95% yield).

¹H NMR (CDCl₃, 400 MHz) δ 1.44 (9H, s), 1.71 (6H, s), 3.19 (2H, d, J=9.7 Hz), 4.20 (2H, s), 7.85 (1H, s)

### Intermediate 11

### Methyl 3-[[[1-[(2,4-dimethoxyphenyl)methylamino]isoquinolin-5-yl]amino]methyl]bicyclo[1.1.1]pentane-1-carboxylate

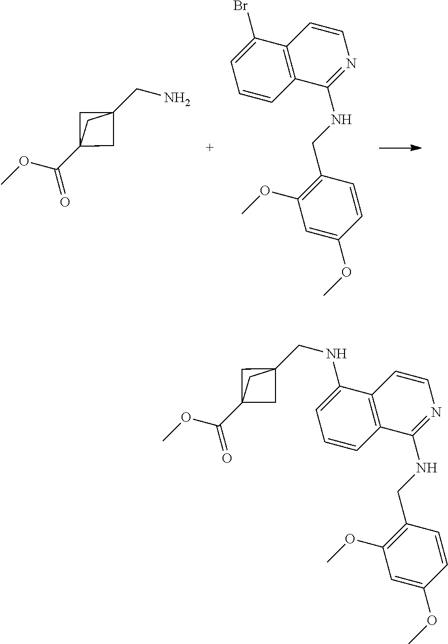 [SMILES: COC(=O)C12CC(CN)(C1)C2 ; COc1ccc(CNc2nccc3c(Br)cccc23)c(OC)c1 ; COC(=O)C12CC(CNc3cccc4c(NCc5ccc(OC)cc5OC)nccc34)(C1)C2]

Following General Method 4, 5-bromo-N-[(2,4-dimethoxyphenyl)methyl]isoquinolin-1-amine (389 mg, 1.04 mmol) was reacted with methyl 3-(aminomethyl)bicyclo[1.1.1]pentane-1-carboxylate hydrochloride (200 mg, 1.04 mmol) in the presence of Cs₂CO₃ (1.03 g, 3.13 mmol) in 1,4-dioxane (5 mL) at 60° C. in a sealed vial for 2 days. The reaction was concentrated onto silica and purified by flash chromatography (silica, 0-43% EtOAc in Pet ether) to afford the product (392 mg, 84% yield).

[M+H]⁺=448.2

### Intermediate 12

### [3-[[[1-[(2,4-Dimethoxyphenyl)methylamino]isoquinolin-5-yl]amino]methyl]-1-bicyclo[1.1.1]pentanyl]methanol

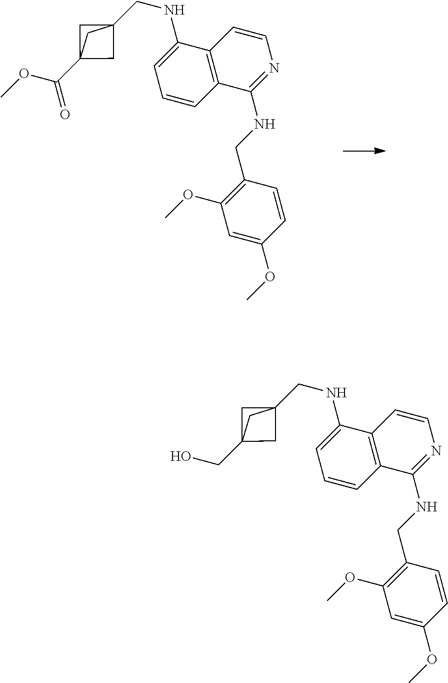 [SMILES: COC(=O)C12CC(CNc3cccc4c(NCc5ccc(OC)cc5OC)nccc34)(C1)C2 ; COc1ccc(CNc2nccc3c(NCC45CC(CO)(C4)C5)cccc23)c(OC)c1]

Following General Method 3b, methyl 3-[[[1-[(2,4-dimethoxyphenyl)methylamino]isoquinolin-5-yl]amino]methyl]bicyclo[1.1.1]pentane-1-carboxylate (946 mg, 2.11 mmol) was reduced over 75 min. The product (852 mg, 96% yield) was used without purification.

[M+H]⁺=420.2

¹H NMR (CDCl₃, 400 MHz) δ 1.74 (6H, s), 3.33 (2H, d, J=5.3 Hz), 3.63 (2H, s), 3.72-3.76 (1H, m), 3.80 (3H, s), 3.85 (3H, s), 4.21 (1H, t, J=5.5 Hz), 4.72 (2H, d, J=5.3 Hz), 5.62 (1H, t, J=5.3 Hz), 6.44 (1H, dd, J=8.2, 2.4 Hz), 6.50 (1H, d, J=2.4 Hz), 6.67 (1H, d, J=7.7 Hz), 6.80 (1H, dd, J=6.3, 0.9 Hz), 7.00-7.09 (1H, m), 7.22-7.28 (1H, m), 7.30 (1H, d, J=8.1 Hz), 8.01 (1H, d, J=6.1 Hz)

### Intermediate 13

### [3-[[[1-[(2,4-Dimethoxyphenyl)methylamino]isoquinolin-5-yl]amino]methyl]-1-bicyclo[1.1.1]pentanyl]methyl methanesulfonate

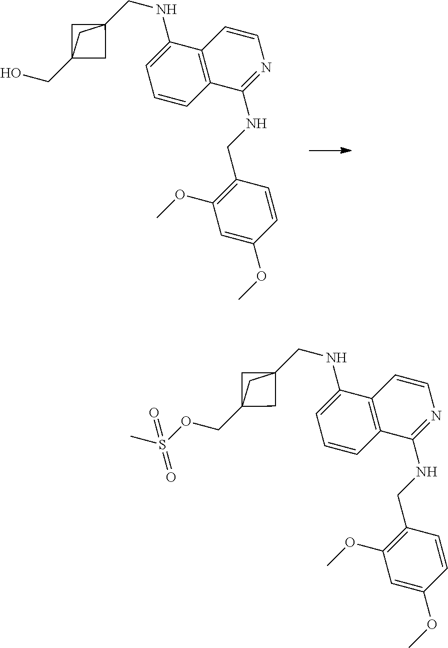 [SMILES: COc1ccc(CNc2nccc3c(NCC45CC(CO)(C4)C5)cccc23)c(OC)c1 ; COc1ccc(CNc2nccc3c(NCC45CC(COS(C)(=O)=O)(C4)C5)cccc23)c(OC)c1]

Using General Method 1a, [3-[[[1-[(2,4-dimethoxyphenyl)methylamino]isoquinolin-5-yl]amino]methyl]-1-bicyclo[1.1.1]pentanyl]methanol (450 mg, 1.07 mmol) was reacted at 0° C. for 90 min to afford the product (510 mg, 86% yield).

[M+H]⁺=498.2

### Intermediate 14

### [4-[[[1-[(2,4-Dimethoxyphenyl)methylamino]isoquinolin-5-yl]amino]methyl]-2-oxabicyclo[2.1.1]hexan-1-yl]methanol

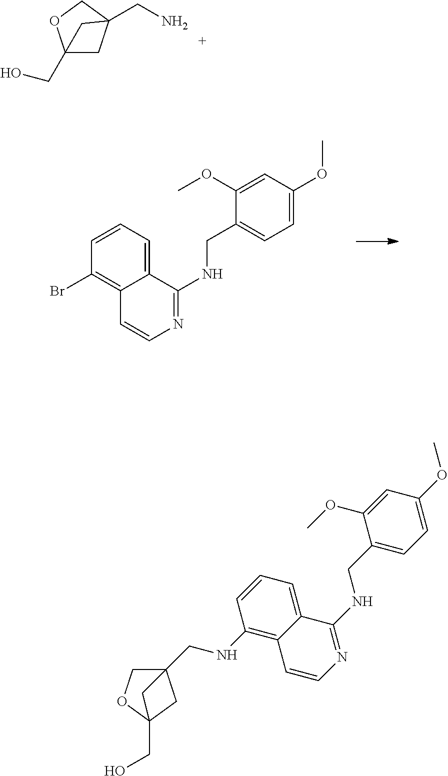 [SMILES: NCC12COC(CO)(C1)C2 ; COc1ccc(CNc2nccc3c(Br)cccc23)c(OC)c1 ; COc1ccc(CNc2nccc3c(NCC45COC(CO)(C4)C5)cccc23)c(OC)c1]

According General Method 4, a suspension of [4-(aminomethyl)-2-oxabicyclo[2.1.1]hexan-1-yl]methanol (500 mg, 3.49 mmol), 5-bromo-N-[(2,4-dimethoxyphenyl)methyl]isoquinolin-1-amine (1434 mg, 3.84 mmol) and NaOtBu (503 mg, 5.24 mmol) in 1,4-dioxane (5 mL) was reacted in the presence of Brettphos Pd G4 (241 mg, 0.26 mmol) in a sealed vial at 40° C. for 18 h and at 60° C. for 2 h. Flash chromatography (Silica, 20-100% EtOAc in Pet. Ether) afforded the product (861 mg, 57% yield).

[M+H]⁺=436.2

### Intermediate 15

### [4-[[[1-[(2,4-Dimethoxyphenyl)methylamino]isoquinolin-5-yl]amino]methyl]-2-oxabicyclo[2.1.1]hexan-1-yl]methyl methanesulfonate

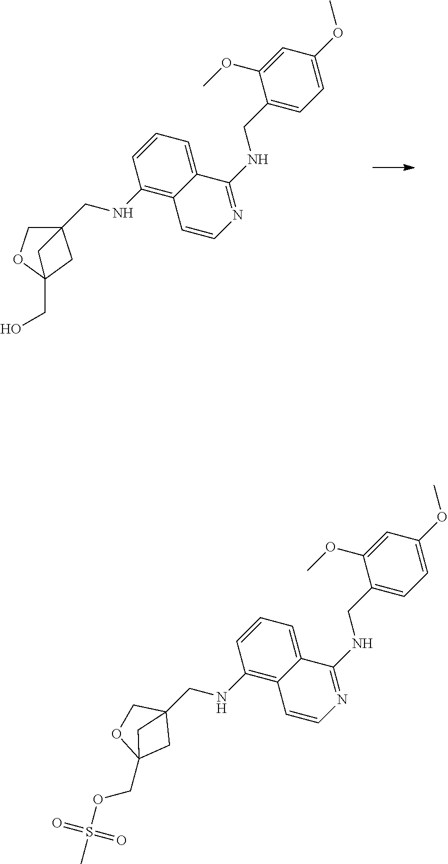 [SMILES: COc1ccc(CNc2nccc3c(NCC45COC(CO)(C4)C5)cccc23)c(OC)c1 ; COc1ccc(CNc2nccc3c(NCC45COC(COS(C)(=O)=O)(C4)C5)cccc23)c(OC)c1]

According to General Method 1a, a solution of [4-[[[1-[(2,4-dimethoxyphenyl)methylamino]isoquinolin-5-yl]amino]methyl]-2-oxabicyclo[2.1.1]hexan-1-yl]methanol (800 mg, 1.84 mmol) was reacted with methane sulfonyl chloride (252 mg, 2.2 mmol) to afford the product (914 mg, 97% yield) which was used without purification by chromatography.

[M+H]⁺=514.2

### Intermediate 16

### 2-Methylimidazo[1,2-a]pyridin-7-ol

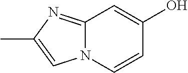

### 2-Methyl-7-phenylmethoxyimidazo[1,2-a]pyridine

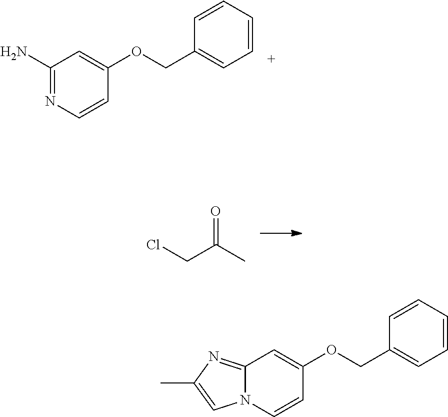

4-(Benzyloxy)pyridin-2-amine (300 mg, 1.5 mmol) was dissolved in EtOH (5 mL). Chloroacetone (131 μL, 1.65 mmol) was added dropwise while cooling in an ice/water bath. The mixture was heated at reflux for 24 h. The solvent was removed in vacuo and the residue dissolved in DCM (10 mL). The organic phase was washed with sat. NaHCO₃ (aq) (2×10 mL), water (2×10 mL), brine (10 mL), dried (MgSO₄) and concentrated. Flash chromatography (SNAP KP-NH (Biotage®), 0-20% MeOH in DCM) afforded the product (131 mg, 37% yield).

¹H NMR (CD₃CN, 400 MHz) δ 2.32 (3H, d, J=0.9 Hz), 5.14 (2H, s), 6.53 (1H, dd, J=7.4, 2.5 Hz), 6.81-6.85 (1H, m), 7.31 (1H, q, J=0.9 Hz), 7.36-7.41 (1H, m), 7.44 (2H, ddd, J=8.0, 7.0, 1.1 Hz), 7.47-7.52 (2H, m), 8.06 (1H, dd, J=7.4, 0.7 Hz) ppm.

### 2-Methylimidazo[1,2-a]pyridin-7-ol

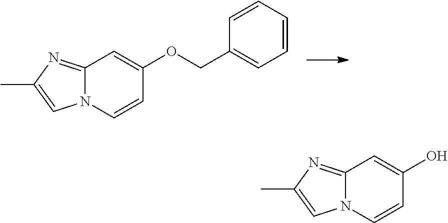 [SMILES: Cc1cn2ccc(OCc3ccccc3)cc2n1 ; Cc1cn2ccc(O)cc2n1]

2-Methyl-7-phenylmethoxyimidazo[1,2-a]pyridine (131 mg, 0.55 mmol) was dissolved in MeOH (13 mL) and subjected to hydrogenation at 50° C. in the H-Cube at 1 mL/min using a 10% Pd/C CatCart. The solvent was removed in vacuo. Flash chromatography (Silica, 0-20% (10% NH₄OH in MeOH) in DCM) afforded 2-methylimidazo[1,2-a]pyridin-7-ol (39 mg, 48% yield) as a colourless oil.

[M+H]⁺=149.0

### Intermediate 17

### [1-[[[1-[(2,4-Dimethoxyphenyl)methylamino]isoquinolin-5-yl]amino]methyl]-2-oxabicyclo[2.1.1]hexan-4-yl]methanol

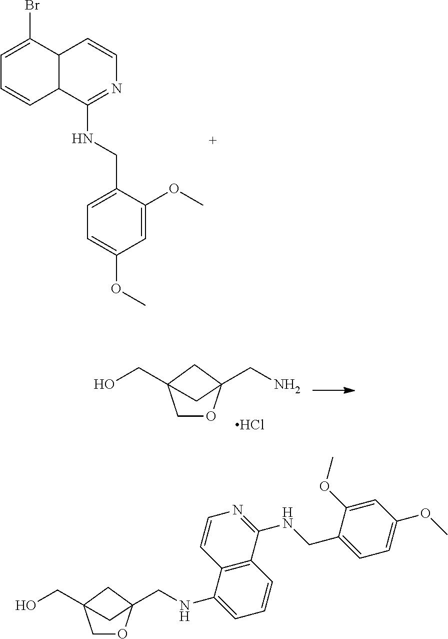 [SMILES: COc1ccc(CNC2=NC=CC3C(Br)=CC=CC23)c(OC)c1 ; NCC12CC(CO)(CO1)C2 ; Cl ; COc1ccc(CNc2nccc3c(NCC45CC(CO)(CO4)C5)cccc23)c(OC)c1]

According to General Method 4, a suspension of [1-(aminomethyl)-2-oxabicyclo[2.1.1]hexan-4-yl]methanol hydrochloride (250 mg, 1.39 mmol), 5-bromo-N-[(2,4-dimethoxyphenyl)methyl]isoquinolin-1-amine (571 mg, 1.53 mmol) and NaOtBu (334 mg, 3.48 mmol) in 1,4-dioxane (5 mL) was reacted in the presence of Brettphos Pd G4 (96 mg, 0.1 mmol) in a sealed vial at 40° C. for 18 h. Flash chromatography (Silica, 20-100% EtOAc in Pet. Ether) afforded the product (429 mg, 71% yield).

[M+H]⁺=436.2

### Intermediate 18

### [1-[[[1-[(2,4-Dimethoxyphenyl)methylamino]isoquinolin-5-yl]amino]methyl]-2-oxabicyclo[2.1.1]hexan-4-yl]methyl methanesulfonate

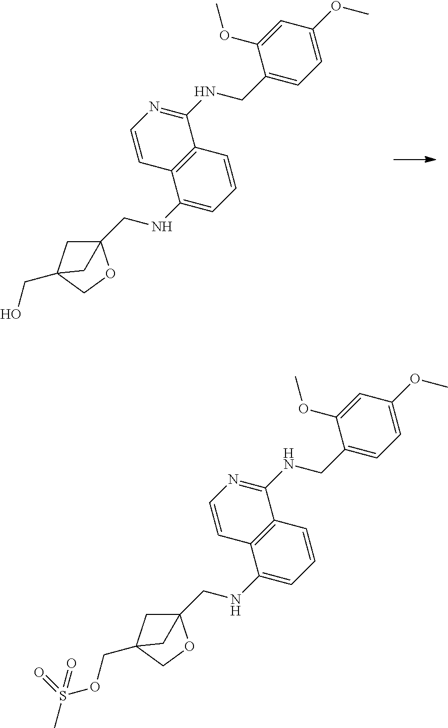 [SMILES: COc1ccc(CNc2nccc3c(NCC45CC(CO)(CO4)C5)cccc23)c(OC)c1 ; COc1ccc(CNc2nccc3c(NCC45CC(COS(C)(=O)=O)(CO4)C5)cccc23)c(OC)c1]

According to General Method 1a, a solution of [1-[[[1-[(2,4-dimethoxyphenyl)methylamino]isoquinolin-5-yl]amino]methyl]-2-oxabicyclo[2.1.1]hexan-4-yl]methanol (429 mg, 0.99 mmol) was reacted with methane sulfonyl chloride (135 mg, 1.18 mmol) to afford the product (440 mg, 87% yield) which was used without purification by chromatography.

LCMS: [M+H]⁺=514.2

### Intermediate 19

### [1-[[[1-[(2,4-Dimethoxyphenyl)methylamino]isoquinolin-5-yl]amino]methyl]-2-oxabicyclo[3.1.1]heptan-5-yl]methanol

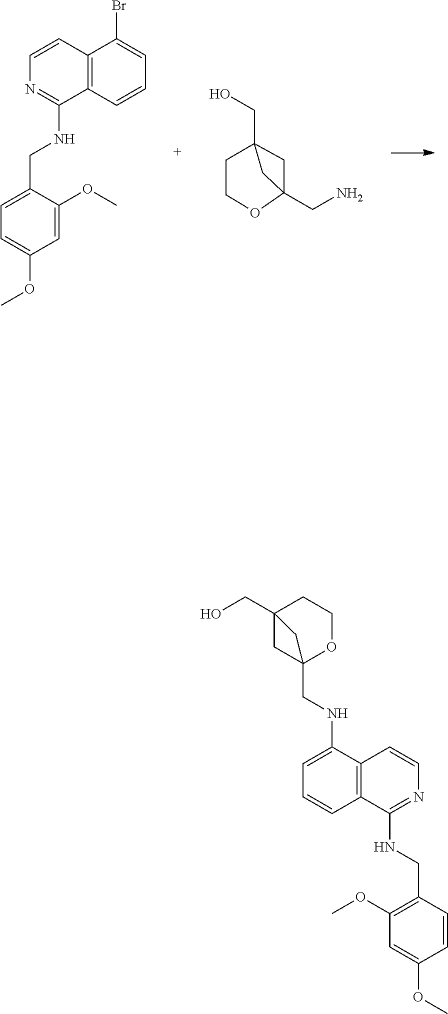 [SMILES: COc1ccc(CNc2nccc3c(Br)cccc23)c(OC)c1 ; NCC12CC(CO)(CCO1)C2 ; COc1ccc(CNc2nccc3c(NCC45CC(CO)(CCO4)C5)cccc23)c(OC)c1]

According General Method 4, a suspension of [1-(aminomethyl)-2-oxabicyclo[3.1.1]heptan-5-yl]methanol hydrochloride (300 mg, 1.55 mmol), 5-bromo-N-[(2,4-dimethoxyphenyl)methyl]isoquinolin-1-amine (578 mg, 1.55 mmol) and NaOtBu (372 mg, 3.87 mmol) in 1,4-dioxane (6 mL) was reacted in the presence of Brettphos Pd G4 (107 mg, 0.12 mmol) in a sealed vial at 40° C. for 3 days and at 60° C. for 18 h. Flash chromatography (Silica, 0-100% EtOAc in Pet. Ether followed by 0-12% MeOH in EtOAc) afforded the product (190 mg, 27% yield).

[M+H]⁺=450.3

### Intermediate 20

### [1-[[[1-[(2,4-Dimethoxyphenyl)methylamino]isoquinolin-5-yl]amino]methyl]-2-oxabicyclo[3.1.1]heptan-5-yl]methyl methanesulfonate

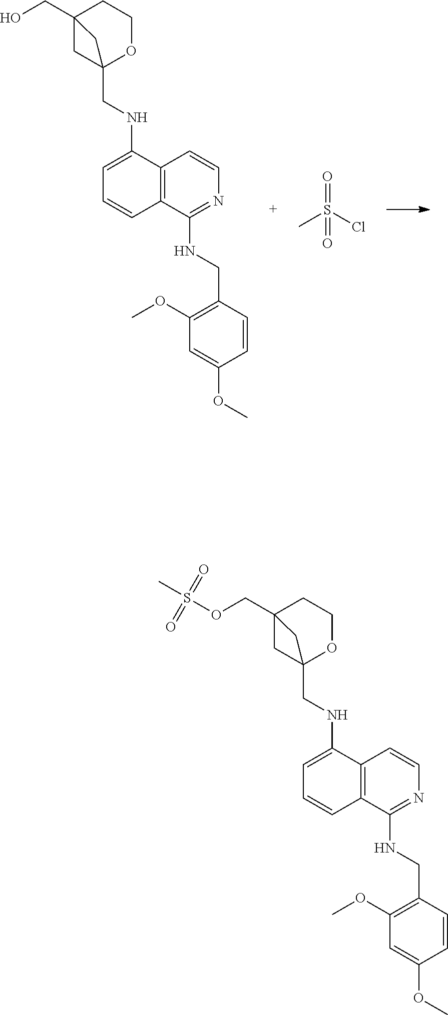

According to General Method 1a, a solution of [1-[[[1-[(2,4-dimethoxyphenyl)methylamino]isoquinolin-5-yl]amino]methyl]-2-oxabicyclo[3.1.1]heptan-5-yl]methanol (160 mg, 0.36 mmol) was reacted with methane sulfonyl chloride (36 μL, 0.46 mmol) to afford the product (190 mg, 91% yield) which was used without purification by chromatography.

[M+H]⁺=528.2

### Intermediate 21

### [5-(Imidazo[1,2-a]pyridin-6-yloxymethyl)-2-oxabicyclo[3.1.1]heptan-1-yl]methanamine

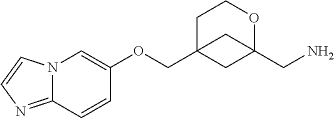 [SMILES: NCC12CC(COc3ccc4nccn4c3)(CCO1)C2]

### tert-Butyl N-[[5-(hydroxymethyl)-2-oxabicyclo[3.1.1]heptan-1-yl]methyl]carbamate

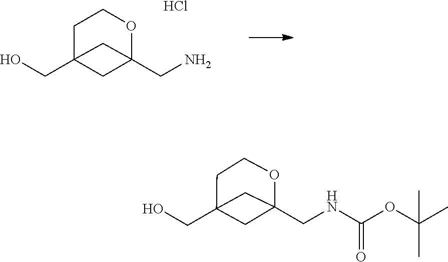 [SMILES: Cl ; NCC12CC(CO)(CCO1)C2 ; CC(C)(C)OC(=O)NCC12CC(CO)(CCO1)C2]

To a solution of [1-(aminomethyl)-2-oxabicyclo[3.1.1]heptan-5-yl]methanol hydrochloride (500 mg, 2.58 mmol) and TEA (1260 μL, 9.04 mmol) in THF (10 mL) at 0° C. was added Boc₂O (676 mg, 3.1 mmol) and the mixture was stirred for 10 min at 0° C. After that time the reaction was stirred for 18 h at rt. The reaction was quenched with water (30 mL), washed with EtOAc (2×50 mL), dried (MgSO₄) and concentrated to afford the product (760 mg, 92% yield).

[M+Na]⁺=280.2

¹H NMR (CDCl₃) δ 1.43 (9H, s), 1.74-1.79 (4H, m), 1.94 (2H, t, J=6.9 Hz), 2.53 (1H, d, J=4.4 Hz), 3.17 (2H, d, J=5.8 Hz), 3.46 (2H, d, J=5.5 Hz), 4.07 (2H, t, J=6.9 Hz), 4.94 (1H, s)

### [1-[[(2-Methylpropan-2-yl)oxycarbonylamino]methyl]-2-oxabicyclo[3.1.1]heptan-5-yl]methyl methanesulfonate

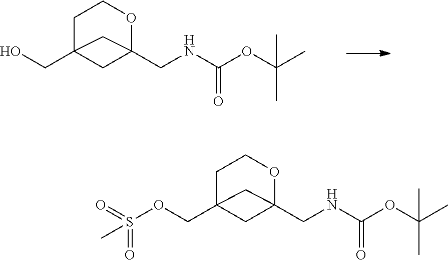 [SMILES: CC(C)(C)OC(=O)NCC12CC(CO)(CCO1)C2 ; CC(C)(C)OC(=O)NCC12CC(COS(C)(=O)=O)(CCO1)C2]

According to a modification of General Method 1a, a solution of tert-butyl N-[[5-(hydroxymethyl)-2-oxabicyclo[3.1.1]heptan-1-yl]methyl]carbamate (760 mg, 2.36 mmol) in DCM (25 mL) was cooled in an ice bath and MsCl (219 μL, 2.84 mmol) was added dropwise followed by TEA (461 L, 3.31 mmol) maintaining the temperature at 0° C. The mixture was stirred at 0° C. for 90 min, after which time it was quenched with water (10 mL) and washed with DCM (2×10 mL), dried (MgSO₄) and concentrated to afford the product (950 mg, 96% yield). [M+Na]⁺=358.3

¹H NMR (CDCl₃) δ 1.44 (9H, s), 1.81-1.89 (4H, m), 2.00 (2H, t, J=6.8 Hz), 3.02 (3H, s), 3.18 (2H, d, J=6.0 Hz), 4.06-4.10 (4H, m), 4.90 (1H, s)

### tert-Butyl N-[[5-(imidazo[1,2-a]pyridin-6-yloxymethyl)-2-oxabicyclo[3.1.1]heptan-1-yl]methyl]carbamate

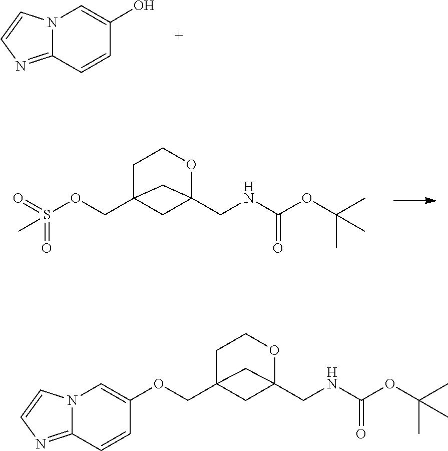 [SMILES: Oc1ccc2nccn2c1 ; CC(C)(C)OC(=O)NCC12CC(COS(C)(=O)=O)(CCO1)C2 ; CC(C)(C)OC(=O)NCC12CC(COc3ccc4nccn4c3)(CCO1)C2]

According to a modification of General Method 2b, to a mixture of [1-[[(2-methylpropan-2-yl)oxycarbonylamino]methyl]-2-oxabicyclo[3.1.1]heptan-5-yl]methyl methanesulfonate (950 mg, 2.27 mmol) and imidazo[1,2-a]pyridin-6-ol (304 mg, 2.27 mmol) in DMF (8 mL) was added K₂CO₃ (939 mg, 6.8 mmol) at rt and the mixture was heated at 60° C. for 12 h. The reaction was cooled to rt, quenched with water (20 mL) and extracted into EtOAc (2×40 mL). The organic layers were combined, washed with water (3×10 mL), brine (10 mL), dried (MgSO₄) and concentrated. The residue was purified by flash chromatography (Silica, 10-100% EtOAc in Pet. Ether and 0-20% MeOH in EtOAc) to afford the product (425 mg, 50% yield).

[M+H]⁺=374.2

¹H NMR (CDCl₃) δ 1.45 (9H, s), 1.92 (4H, s), 2.07 (2H, t, J=6.9 Hz), 3.23 (2H, d, J=5.9 Hz), 3.74 (2H, s), 4.09-4.14 (2H, m), 5.03 (1H, t, J=5.9 Hz), 6.96 (1H, dd, J=9.7, 2.3 Hz), 7.48-7.51 (2H, m), 7.56 (1H, d, J=0.8 Hz), 7.66 (1H, d, J=1.8 Hz)

### [5-(Imidazo[1,2-a]pyridin-6-yloxymethyl)-2-oxabicyclo[3.1.1]heptan-1-yl]methanamine

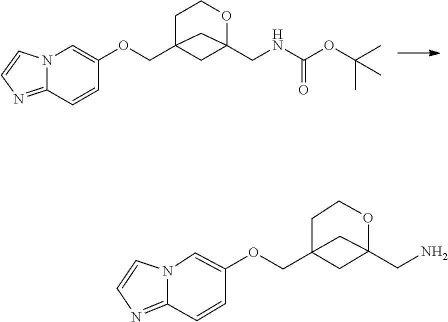 [SMILES: CC(C)(C)OC(=O)NCC12CC(COc3ccc4nccn4c3)(CCO1)C2 ; NCC12CC(COc3ccc4nccn4c3)(CCO1)C2]

According to a modification of General Method 5b, a solution of tert-butyl N-[[5-(imidazo[1,2-a]pyridin-6-yloxymethyl)-2-oxabicyclo[3.1.1]heptan-1-yl]methyl]carbamate (125 mg, 0.33 mmol) in DCM (3 mL) and TFA (494 μL, 6.46 mmol) was stirred at rt for 2 h, after which time it was concentrated. The mixture was taken up in MeOH and passed directly through an SCX and washed with MeOH. The product was eluted with a solution of 7M NH₃ in MeOH and concentrated to afford the product (85.0 mg, 93% yield).

[M+H]⁺=274.1

¹H NMR (CDCl₃) δ 1.51 (2H, s), 1.81-1.86 (2H, m), 1.91-1.97 (2H, m), 2.08 (2H, t, J=6.8 Hz), 2.73 (2H, s), 3.75 (2H, s), 4.15 (2H, t, J=6.8 Hz), 6.97 (1H, dd, J=9.8, 2.3 Hz), 7.48-7.51 (2H, m), 7.56 (1H, d, J=0.9 Hz), 7.66 (1H, d, J=2.4 Hz)

### Intermediate 22

### 8-Methylimidazo[1,2-a]pyridin-7-ol

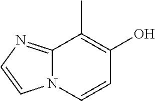

### 7-Bromo-8-methylimidazo[1,2-a]pyridine

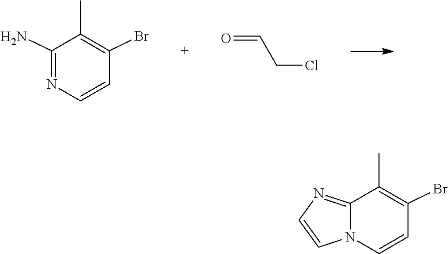

According to a modification of General Method 12, to a solution of 4-bromo-3-methylpyridin-2-amine (470 mg, 2.51 mmol) in EtOH (3 mL) were added NaHCO₃(422 mg, 5.03 mmol) and chloroacetaldehyde (638 μL, 5.03 mmol). The mixture was stirred for 20 h at 75° C. The mixture was cooled to rt, filtered through Celite washing with EtOAc (50 mL) and concentrated. Flash chromatography (Silica, 50-100% EtOAc in Pet. Ether) afforded the product (431 mg, 81% yield).

[M+H]⁺=211.0, 213.0

### 8-Methylimidazo[1,2-a]pyridin-7-ol

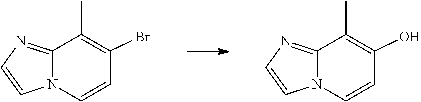

According to a modification of General Method 13, 7-bromo-8-methylimidazo[1,2-a]pyridine (431 mg, 2.04 mmol), bis(pinacolato)diboron (1167 mg, 4.59 mmol) and [1,1-bis(diphenylphosphino)ferrocene]dichloropalladium(II) (149 mg, 0.2 mmol) were added to a screw capped vial. The vial was flushed with N₂ (g) before 1,4-dioxane (5 mL) was added. The mixture was purged with N₂ (g) for 5 min before KOAc (607 mg, 6.13 mmol) was added. The mixture was purged for a further 5 min and stirred at 100° C. for 18 h. The mixture was cooled to rt before AcOH (234 μL, 4.08 mmol) and water (1 mL) were added. The mixture was stirred for 15 min before a solution of H₂O₂ in water (30% w/w, 417 μL, 4.08 mmol) was added. The mixture was stirred for 3 h. Na₂S₂O₃ (5 mg) was added and the mixture filtered through Celite and concentrated. The product was passed directly through SCX washing with MeOH. The product was eluted with a solution of 1M NH₃ in MeOH and concentrated to afford the product (92 mg, 30%) as a brown solid.

[M+H]⁺=149.0

### Intermediate 23

### 3-Methylimidazo[1,2-a]pyridin-6-ol

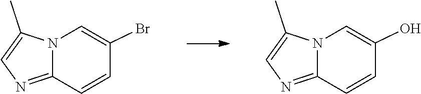

According to a modification of General Method 13, 6-bromo-3-methylimidazo[1,2-a]pyridine (525 mg, 2.49 mmol) and bis(pinacolato)diboron (1.42 g, 5.6 mmol) were suspended in 1,4-dioxane (5 mL). The mixture was purged with N₂ (g) then [1,1-bis(diphenylphosphino)ferrocene]dichloropalladium(II) (181 mg, 0.25 mmol) was added followed by KOAc (740 mg, 7.46 mmol). The mixture was purged for a further 5 min and stirred at 100° C. for 5 h. The mixture was cooled to rt before AcOH (285 μL, 4.97 mmol) and water (1 mL) were added. The mixture was stirred for 15 min before a solution of H₂O₂ in water (30% w/w, 508 μL, 4.97 mmol) was added dropwise at 0° C. The mixture was stirred for 90 min at rt, quenched with solid Na₂S₂O₃ (5 mg), filtered through Celite® and concentrated. The product was passed directly through SCX and washed with MeOH. The product was eluted with a solution of 1M NH₃ in MeOH and concentrated to afford the product (160 mg, 33% yield).

[M+H]⁺=149.1

¹H NMR (DMSO, 400 MHz) δ 2.36 (3H, s), 6.92 (1H, dd, J=9.6, 2.3 Hz), 7.25 (1H, s), 7.40 (1H, d, J=9.5 Hz), 7.59 (1H, d, J=1.9 Hz), 9.45 (1H, s)

### Intermediate 24

### 4-((4-(((1-((2,4-Dimethoxybenzyl)amino)isoquinolin-5-yl)amino)methyl)-2-azabicyclo[2.1.1]hexan-1-yl)methoxy)-1-methylpyridin-2(1H)-one

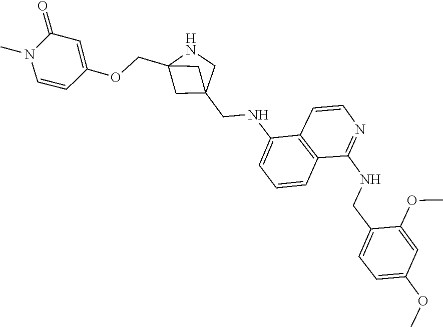

### 2-O-Benzyl 1-O-methyl 4-(azidomethyl)-2-azabicyclo[2.1.1]hexane-1,2-dicarboxylate

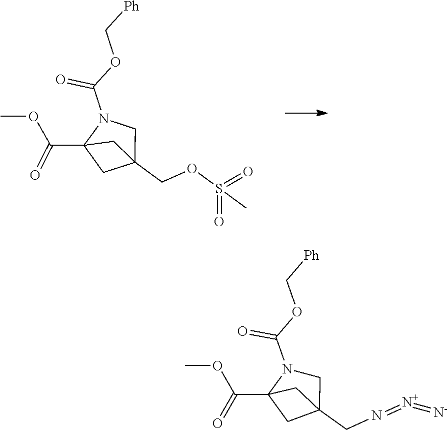 [SMILES: COC(=O)C12CC(COS(C)(=O)=O)(CN1C(=O)OCc1ccccc1)C2 ; COC(=O)C12CC(CN=[N+]=[N-])(CN1C(=O)OCc1ccccc1)C2]

To 2-O-benzyl 1-O-methyl 4-(methylsulfonyloxymethyl)-2-azabicyclo[2.1.1]hexane-1,2-dicarboxylate (458 mg, 1.19 mmol) in DMF (5 mL) was added NaN₃ (155 mg, 2.39 mmol) at rt. The mixture was stirred at rt for 4 days. The mixture was diluted with EtOAc (50 mL) and washed with water (4×10 mL) and brine (10 mL), dried (MgSO₄) and concentrated to afford the product (330 mg, 75% yield).

[M+H]⁺=331.1

¹H NMR (CD₃CN) δ 1.73 (2H, dd, J=4.7, 1.9 Hz), 2.01 (2H, dd, J=4.6, 1.4 Hz), 3.38 (2H, s), 3.56 (5H, s), 5.04 (2H, s), 7.28-7.37 (5H, m)

### 2-O-Benzyl 1-O-methyl 4-(azidomethyl)-2-azabicyclo[2.1.1]hexane-1,2-dicarboxylate

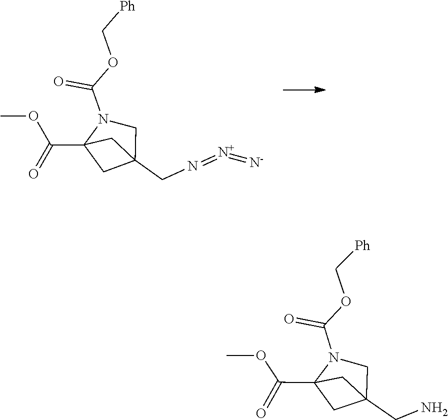 [SMILES: COC(=O)C12CC(CN=[N+]=[N-])(CN1C(=O)OCc1ccccc1)C2 ; COC(=O)C12CC(CN)(CN1C(=O)OCc1ccccc1)C2]

2-O-Benzyl 1-O-methyl 4-(azidomethyl)-2-azabicyclo[2.1.1]hexane-1,2-dicarboxylate (330 mg, 0.9 mmol) was dissolved in THF (6 mL). Triphenylphosphine, polymer bound (3 mmol/1 g of resin) (453 mg, 1.35 mmol) was added and the reaction stirred at rt for 2 h. Water (162 μL, 8.99 mmol) was added and the reaction heated at reflux for 3 h. The mixture was cooled to rt and filtered, washing the resin with 10% MeOH in DCM (25 mL). The filtrate was concentrated to afford the product (240 mg, 79% yield).

[M+H]⁺=305.1

¹H NMR (CDCl₃) δ 1.29 (2H, br s), 1.70 (2H, dd, J=4.6, 1.7 Hz), 1.90 (2H, d, J=4.6 Hz), 2.85 (2H, s), 3.34 (2H, s), 3.53 (3H, br s), 5.02 (2H, s), 7.19-7.28 (5H, m)

### 2-O-benzyl 1-O-methyl 4-[[[1-[(2,4-dimethoxyphenyl)methylamino]isoquinolin-5-yl]amino]methyl]-2-azabicyclo[2.1.1]hexane-1,2-dicarboxylate

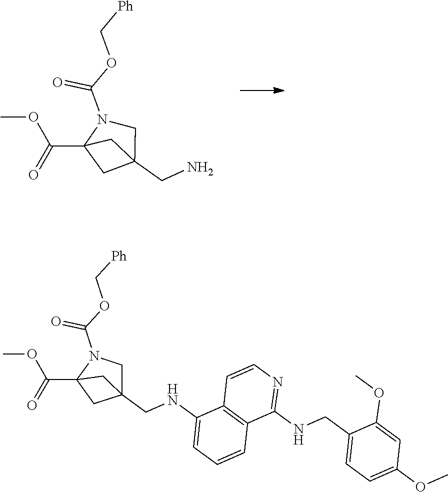

According to general method 4, 5-bromo-N-(2,4-dimethoxybenzyl)isoquinolin-1-amine (2239 mg, 6.00 mmol) was reacted with 2-O-benzyl 1-O-methyl 4-(aminomethyl)-2-azabicyclo[2.1.1]hexane-1,2-dicarboxylate (1927 mg, 5.70 mmol), Cs₂CO₃ (5610 mg, 17.0 mmol) and Brettphos Pd G4 (801 mg, 0.852 mmol) in 1,4-dioxane (25 mL) under nitrogen. The reaction mixture was heated to 65° C. in a sealed vial for 6 h. The mixture was cooled, filtered (Celite) washing with EtOAc (200 ml), and the filtrate was concentrated. The residue was purified by flash chromatography (Silica, 0-70% EtOAc in hexanes) to afford the product (2580 mg, 76%).

[M+H]⁺=597.3

¹H NMR (CDCl₃, 500 MHz) δ 1.91 (2H, dd, J=4.7, 1.9 Hz), 2.11 (2H, d, J=4.8 Hz), 3.51-3.70 (7H, m), 3.80 (3H, s), 3.86 (3H, s), 4.26 (1H, s), 4.72 (2H, d, J=5.3 Hz), 5.12 (2H, s), 5.63 (1H, t, J=5.3 Hz), 6.45 (1H, dd, J=8.3, 2.4 Hz), 6.50 (1H, d, J=2.4 Hz), 6.66 (1H, d, J=7.7 Hz), 6.75 (1H, d, J=6.1 Hz), 7.08 (1H, d, J=8.3 Hz), 7.28-7.39 (7H, m), 8.02 (1H, d, J=6.1 Hz).

### Benzyl 4-[[[1-[(2,4-dimethoxyphenyl)methylamino]-5-isoquinolyl]amino]methyl]-1-(hydroxymethyl)-2-azabicyclo[2.1.1]hexane-2-carboxylate

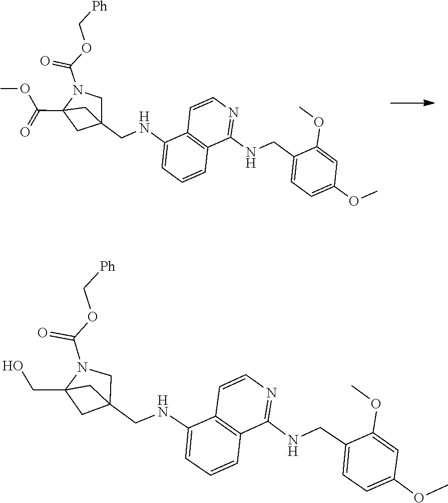

Lithium triethylborohydride (1 M in THF, 7.2 mL, 7.20 mmol) was added to a mixture of 2-O-benzyl 1-O-methyl 4-[[[1-[(2,4-dimethoxyphenyl)methylamino]-5-isoquinolyl]amino]methyl]-2-azabicyclo[2.1.1]hexane-1,2-dicarboxylate (1713 mg, 2.87 mmol) in anhydrous THF (15 mL) at 0° C. The mixture was stirred at 0° C. for 2.5 h, and diluted with water (10 mL), brine (20 mL) and EtOAc (90 mL). The phases were separated, and the aq phase was extracted with EtOAc (2×50 mL). The combined organic phases were dried (MgSO₄) and concentrated. The residue was purified by flash chromatography (Silica, 20-68% EtOAc in hexanes) to afford the product (1096 mg, 67% yield).

[M+H]⁺=569.8

¹H NMR (CDCl₃, 400 MHz) δ 1.78 (4H, s), 3.51 (2H, s), 3.54 (2H, s), 3.80 (3H, s), 3.86 (3H, s), 3.98 (2H, d, J=7.4 Hz), 4.24 (1H, s), 4.54 (1H, s), 4.72 (2H, d, J=5.3 Hz), 5.14 (2H, s), 5.63 (1H, t, J=5.4 Hz), 6.45 (1H, dd, J=8.2, 2.4 Hz), 6.50 (1H, d, J=2.4 Hz), 6.67 (1H, dd, J=7.8, 0.8 Hz), 6.76 (1H, dd, J=6.1, 0.9 Hz), 7.05-7.11 (1H, m), 7.37 (7H, d, J=4.6 Hz), 8.02 (1H, d, J=6.1 Hz).

### Benzyl 4-[[[1-[(2,4-dimethoxyphenyl)methylamino]-5-isoquinolyl]amino]methyl]-1-[(1-methyl-2-oxo-4-pyridyl)oxymethyl]-2-azabicyclo[2.1.1]hexane-2-carboxylate

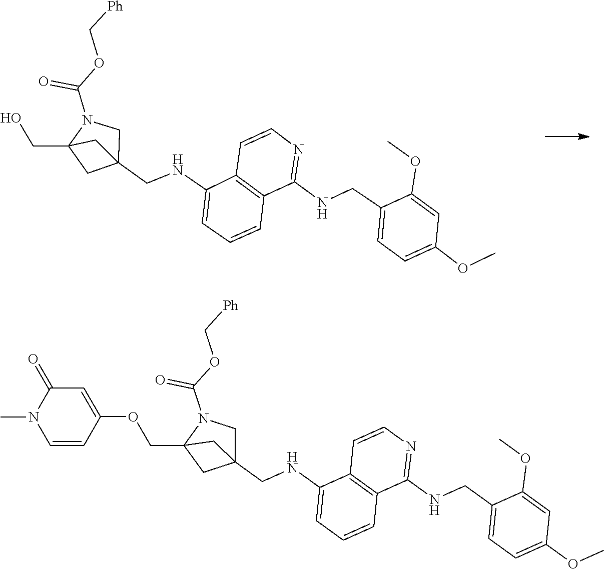

DIAD (550 μL, 2.74 mmol) was added to a mixture of benzyl 4-[[[1-[(2,4-dimethoxyphenyl)methylamino]-5-isoquinolyl]amino]methyl]-1-(hydroxymethyl)-2-azabicyclo[2.1.1]hexane-2-carboxylate (1046 mg, 1.84 mmol), triphenylphosphine (730 mg, 2.75 mmol) and 4-hydroxy-1-methyl-pyridin-2-one (254 mg, 1.93 mmol) in anhydrous THF (15 mL) at rt. The mixture was stirred at rt for 23 h and concentrated. The residue was diluted with NaOH(aq) (1M, 20 mL) and DCM (50 mL). The phases were separated, and the aq phase was extracted with DCM (2×50 mL). The combined organic phases were washed with sat. NaHCO₃ (aq) (25 mL) and brine (30 mL), dried (Na₂SO₄), filtered and concentrated in vacuo. The residue was purified by flash column chromatography (Silica, 0-20% MeOH in EtOAc) to afford the product (319 mg, 26% yield).

[M+H]⁺=676.4

¹H NMR (CDCl₃, 400 MHz) δ 1.73 (2H, dd, J=4.5, 1.9 Hz), 2.01-2.04 (2H, m), 3.46 (3H, s), 3.55 (2H, s), 3.57 (3H, s), 3.80 (3H, s), 3.86 (3H, s), 4.26 (1H, s), 4.47-4.56 (2H, m), 4.73 (2H, d, J=5.3 Hz), 5.10 (2H, s), 5.62 (1H, d, J=5.5 Hz), 5.82-5.94 (2H, m), 6.45 (1H, dd, J=8.2, 2.4 Hz), 6.50 (1H, d, J=2.4 Hz), 6.68 (1H, d, J=7.7 Hz), 6.77 (1H, dd, J=6.2, 0.9 Hz), 7.08 (2H, dd, J=8.0, 4.4 Hz), 7.28-7.39 (6H, m), 8.03 (1H, d, J=6.1 Hz).

### 4-[[4-[[[1-[(2,4-Dimethoxyphenyl)methylamino]-5-isoquinolyl]amino]methyl]-2-azabicyclo[2.1.1]hexan-1-yl]methoxy]-1-methyl-pyridin-2-one

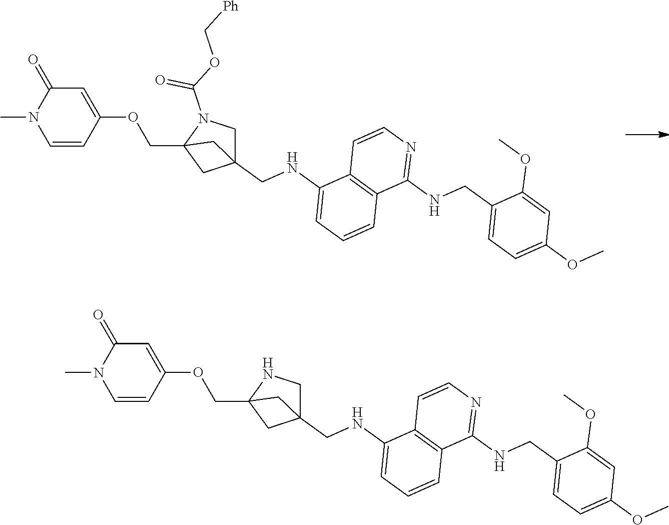

A solution of benzyl 4-[[[1-[(2,4-dimethoxyphenyl)methylamino]-5-isoquinolyl]amino]methyl]-1-[(1-methyl-2-oxo-4-pyridyl)oxymethyl]-2-azabicyclo[2.1.1]hexane-2-carboxylate (319 mg, 0.472 mmol) in MeOH (3 mL) was added to a mixture of Pd(OH)₂/C (20 wt %, 82.0 mg, 0.0117 mmol) in MeOH (1.8 mL). The mixture was subjected to hydrogenation at 1 atmosphere and rt for 4 h. The mixture was filtered over Celite washing with MeOH (2×25 mL), and the filtrate was concentrated to afford the product (234 mg, 91%).

[M+H]⁺=542.4

¹H NMR (DMSO-d6, 500 MHz) δ 1.34 (2H, dd, J=4.0, 1.7 Hz), 1.62 (2H, dd, J=4.2, 1.6 Hz), 2.83 (2H, s), 3.17 (1H, d, J=2.6 Hz), 3.31 (3H+H2O, s), 3.49 (2H, d, J=5.8 Hz), 3.71 (3H, s), 3.82 (3H, s), 4.03 (2H, s), 4.58 (2H, d, J=5.6 Hz), 5.76 (1H, d, J=2.8 Hz), 5.89 (1H, dd, J=7.5, 2.8 Hz), 5.95 (1H, t, J=6.0 Hz), 6.39 (1H, dd, J=8.4, 2.4 Hz), 6.55 (1H, d, J=2.4 Hz), 6.66 (1H, d, J=7.7 Hz), 7.02 (1H, d, J=8.4 Hz), 7.12 (1H, d, J=6.2 Hz), 7.23 (1H, t, J=8.0 Hz), 7.36 (1H, t, J=5.8 Hz), 7.42 (1H, d, J=8.4 Hz), 7.52 (1H, d, J=7.6 Hz), 7.72 (1H, d, J=6.0 Hz).

### Intermediate 25

### N1-(2,4-Dimethoxybenzyl)isoquinoline-1,5-diamine

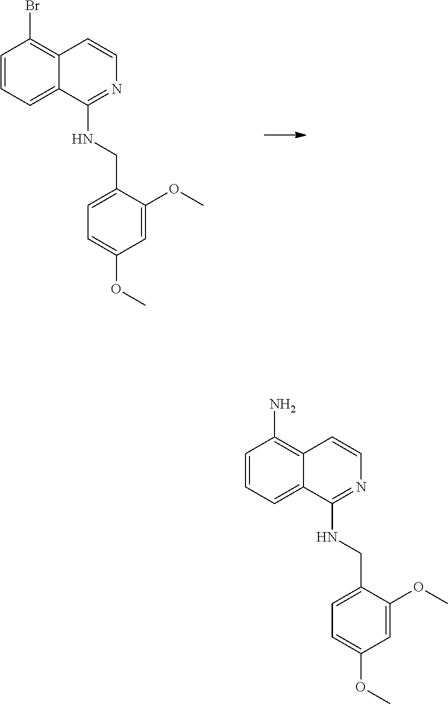 [SMILES: COc1ccc(CNc2nccc3c(Br)cccc23)c(OC)c1 ; COc1ccc(CNc2nccc3c(N)cccc23)c(OC)c1]

A mixture of 5-bromo-N-[(2,4-dimethoxyphenyl)methyl]isoquinolin-1-amine (746 mg, 2.0 mmol), 2,2,2-trifluoroacetamide (339 mg, 3.0 mmol), CuI₂ (38 mg, 0.2 mmol), K₂CO₃ (553 mg, 4.0 mmol) and N,N′-dimethylethylenediamine (35 mg, 0.4 mmol) were combined in a reaction vial. Anhydrous 1,4-dioxane (7 mL) was added and the suspension purged with N₂(g) before being capped and heated to 75° C. for 24 h. The reaction was recharged with 2,2,2-trifluoroacetamide (339 mg, 3.0 mmol), CuI₂ (38 mg, 0.2 mmol), K₂CO₃ (553 mg, 4.0 mmol) and N,N′-dimethylethylenediamine (35 mg, 0.4 mmol). The mixture was degassed with N₂(g) and heated at 70° C. for 18 h. Water (3 mL) was added and the reaction heated at 80° C. for 6 h. The mixture was partitioned between EtOAc (30 mL) and water (10 mL). The aqueous layer was extracted with EtOAc (2×30 mL) and the combined organics were washed with brine (50 mL), dried (Na₂SO₄), filtered and concentrated. Flash chromatography (Silica, 0-100% EtOAc/DCM) afforded the product (492 mg, 72% yield).

[M+H]⁺=310.1

¹H NMR (CDCl₃, 400 MHz) δ 3.79 (3H, s), 3.85 (3H, s), 4.10 (2H, br s), 4.72 (2H, d, J=5.3 Hz), 5.58-5.67 (1H, m), 6.44 (1H, dd, J=8.2, 2.4 Hz), 6.49 (1H, d, J=2.4 Hz), 6.78-6.86 (2H, m), 7.12 (1H, dt, J=8.4, 1.1 Hz), 7.21 (1H, dd, J=8.4, 7.4 Hz), 7.30 (1H, d, J=8.2 Hz), 8.01 (1H, d, J=6.1 Hz)

### Intermediate 26

### 4-((1-(((1-((2,4-Dimethoxybenzyl)amino)isoquinolin-5-yl)amino)methyl)-2-azabicyclo[2.1.1]hexan-4-yl)methoxy)-1-methylpyridin-2(1H)-one

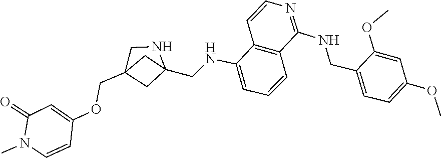

### Methyl 4-(hydroxymethyl)-2-azabicyclo[2.1.1]hexane-1-carboxylate; hydrochloride

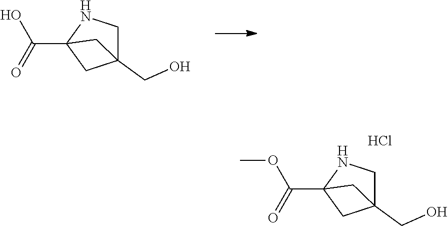

The product was prepared from 4-(hydroxymethyl)-2-azabicyclo[2.1.1]hexane-1-carboxylic acid (CAS 1522098-73-2, Tetrahedron Letters 55 (2014) 466-468) following the procedure described in ChemistrySelect 2019, 4, 4933-493.

### 2-O-Benzyl 1-O-methyl 4-(hydroxymethyl)-2-azabicyclo[2.1.1]hexane-1,2-dicarboxylate

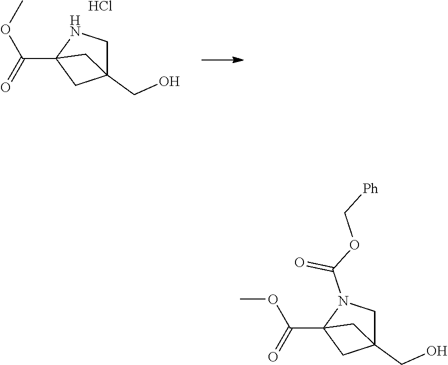 [SMILES: COC(=O)C12CC(CO)(CN1)C2 ; Cl ; COC(=O)C12CC(CO)(CN1C(=O)OCc1ccccc1)C2]

Methyl 4-(hydroxymethyl)-2-azabicyclo[2.1.1]hexane-1-carboxylate hydrochloride (600 mg, 2.89 mmol) was dissolved in DCM (15 mL) and cooled to 0° C. TEA (805 μL, 5.78 mmol) was added followed by benzyl chloroformate (407 μL, 2.89 mmol). The reaction was stirred at rt overnight before retreating with benzyl chloroformate (200 μL, 1.42 mmol) at 0° C. and stirring at rt for an additional 1 h. DCM (30 mL) and water (10 mL) were added. The organic layer was dried (MgSO₄), filtered and concentrated. The crude product was purified by flash chromatography (Silica, 20-100% EtOAc in Pet. Ether) to afford the product (465 mg, 53% yield) as a colourless oil.

[M+H]⁺=306.2

¹H NMR (DMSO-d6, 400 MHz) δ 1.56-1.66 (2H, m), 1.89-1.97 (2H, m), 3.32 (2H, s), 3.50-3.65 (5H, m), 4.74-4.78 (1H, m), 5.04 (2H, s), 7.30-7.42 (5H, m)

### 2-O-Benzyl 1-O-methyl 4-(methylsulfonyloxymethyl)-2-azabicyclo[2.1.1]hexane-1,2-dicarboxylate

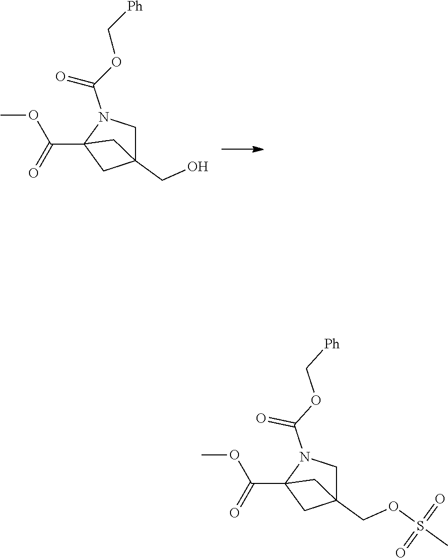

Following a modification of General Method 1a, 2-O-benzyl 1-O-methyl 4-(hydroxymethyl)-2-azabicyclo[2.1.1]hexane-1,2-dicarboxylate (1206 mg, 3.95 mmol) was reacted with MsCl (367 μL, 4.74 mmol) and TEA (771 μL, 5.53 mmol). The mixture was stirred while cooling for 90 min, after which time water (20 mL) was added and the mixture extracted with DCM (2×50 mL), dried (MgSO₄), filtered and concentrated to give the product (1480 mg, 98% yield)

[M+H]⁺=384.1.

1H NMR (CDCl₃, 400 MHz) δ 1.91 (2H, dd, J=4.8, 1.9 Hz), 2.12 (2H, dd, J=5.0, 1.9 Hz), 3.04 (3H, s), 3.51 (2H, s), 3.67 (3H, s), 4.40 (2H, s), 5.11 (2H, s), 7.27-7.44 (5H, m).

### 2-O-Benzyl 1-O-methyl 4-[(1-methyl-2-oxopyridin-4-yl)oxymethyl]-2-azabicyclo[2.1.1]hexane-1,2-dicarboxylate

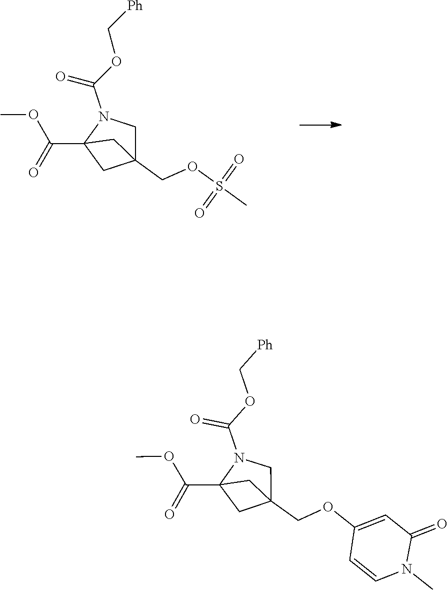

According to a modification of General Method 2b, 2-O-benzyl 1-O-methyl 4-(methylsulfonyloxymethyl)-2-azabicyclo[2.1.1]hexane-1,2-dicarboxylate (703 mg, 1.56 mmol) was reacted with 4-hydroxy-1-methyl-2-pyridone (195 mg, 1.56 mmol) and K₂CO₃ (646 mg, 4.68 mmol) in DMF (10 mL) at 90° C. for 17 h. The reaction mixture was cooled and quenched with water (10 mL). The reaction mixture was extracted with EtOAc (3×20 mL). The organic layers were combined, washed with water (3×20 mL), dried (MgSO₄), and concentrated. The residue was purified by flash chromatography (Silica, 0-9% MeOH in DCM) to afford the product (620 mg, 96% yield).

[M+H]⁺=413.4.

¹H NMR (DMSO-d6, 400 MHz) δ 1.74 (2H, dd, J=4.6, 1.9 Hz), 2.10 (2H, dd, J=4.7, 1.8 Hz), 3.32 (3H, s), 3.45 (2H, s), 3.57 (3H, s), 4.19 (2H, s), 5.06 (2H, s), 5.79 (1H, d, J=2.8 Hz), 5.93 (1H, dd, J=7.6, 2.8 Hz), 7.36 (5H, m), 7.56 (1H, d, J=7.5 Hz)

### Benzyl 1-(hydroxymethyl)-4-[(1-methyl-2-oxopyridin-4-yl)oxymethyl]-2-azabicyclo[2.1.1]hexane-2-carboxylate

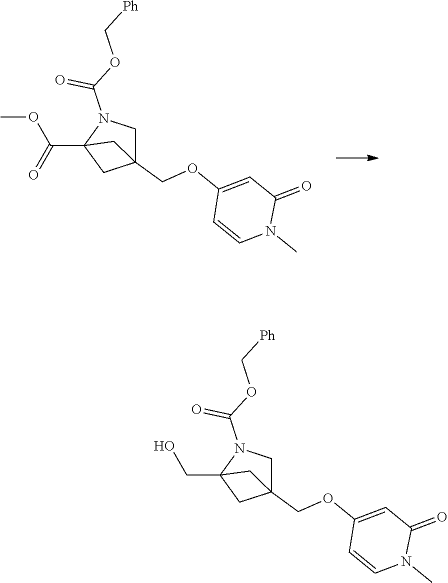 [SMILES: COC(=O)C12CC(COc3ccn(C)c(=O)c3)(CN1C(=O)OCc1ccccc1)C2 ; Cn1ccc(OCC23CN(C(=O)OCc4ccccc4)C(CO)(C2)C3)cc1=O]

2-O-Benzyl 1-O-methyl 4-[(1-methyl-2-oxopyridin-4-yl)oxymethyl]-2-azabicyclo[2.1.1]hexane-1,2-dicarboxylate (582 mg, 1.41 mmol) was dissolved in anhydrous THF (10 mL). The solution was cooled in an ice bath and lithium borohydride (LiBH₄) (2M solution in THF) (776 μL, 1.55 mmol) was added. The reaction mixture was stirred at rt for 72 h after which time water (10 mL) and DCM (30 mL) were added. The organic layer was isolated and the aqueous layer re-extracted with DCM (3×10 mL). The combined organic layers were dried (MgSO₄), filtered and concentrated to afford the product (464 mg, 86% yield).

[M+H]⁺=385.3

¹H NMR (DMSO-d6, 400 MHz) δ 1.46 (2H, dd, J=4.4, 1.8 Hz), 1.83-1.95 (2H, m), 3.33 (3H, s), 3.41 (2H, s), 3.90 (2H, d, J=6.1 Hz), 4.17 (2H, s), 4.70 (1H, t, J=6.2 Hz), 5.06 (2H, s), 5.80 (1H, d, J=2.8 Hz), 5.93 (1H, dd, J=7.5, 2.8 Hz), 7.27-7.36 (1H, m), 7.37 (4H, d, J=3.7 Hz), 7.56 (1H, d, J=7.6 Hz)

### Benzyl 1-formyl-4-[(1-methyl-2-oxopyridin-4-yl)oxymethyl]-2-azabicyclo[2.1.1]hexane-2-carboxylate

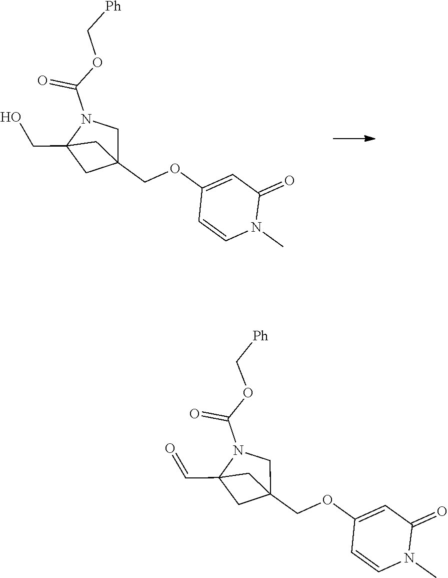 [SMILES: Cn1ccc(OCC23CN(C(=O)OCc4ccccc4)C(CO)(C2)C3)cc1=O ; Cn1ccc(OCC23CN(C(=O)OCc4ccccc4)C(C=O)(C2)C3)cc1=O]

Dess-Martin periodinane (1986 mg, 4.68 mmol) was added to a solution of benzyl 1-(hydroxymethyl)-4-[(1-methyl-2-oxo-4-pyridyl)oxymethyl]-2-azabicyclo[2.1.1]hexane-2-carboxylate (1200 mg, 3.12 mmol) in DCM (30 mL) at 0° C. The mixture was stirred at rt for 1 h. The reaction was quenched with sat. NaHCO₃ (aq) (50 mL) and sat. Na₂S₂O₃ (aq) (50 mL) and extracted with DCM (2×50 mL). The combined organic layers were dried (Na₂SO₄), filtered and concentrated to afford the product (2000 mg, 99% yield).

[M+H]⁺=384.4

¹H NMR (DMSO-d6, 400 MHz) δ 1.73 (2H, dd, J=4.4, 1.8 Hz), 2.05 (2H, d, J=5.7 Hz), 3.32 (3H, s), 3.50 (2H, s), 4.20 (2H, s), 5.12 (2H, s), 5.80 (1H, d, J=2.7 Hz), 5.94 (1H, dd, J=7.5, 2.8 Hz), 7.27-7.44 (5H, m), 7.56 (1H, d, J=7.6 Hz), 9.77 (1H, s)

### Benzyl 1-[[[1-[(2,4-dimethoxyphenyl)methylamino]-5-isoquinolyl]amino]methyl]-4-[(1-methyl-2-oxo-4-pyridyl)oxymethyl]-2-azabicyclo[2.1.1]hexane-2-carboxylate

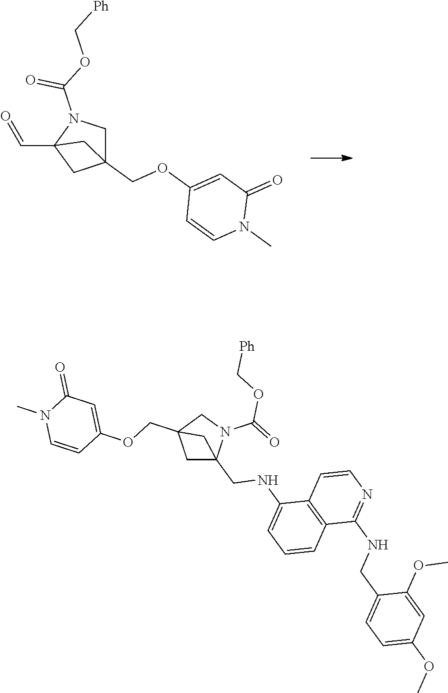

AcOH (269 μL, 4.71 mmol) was added to a mixture of benzyl 1-formyl-4-[(1-methyl-2-oxo-4-pyridyl)oxymethyl]-2-azabicyclo[2.1.1]hexane-2-carboxylate (2000 mg, 60% purity, 3.14 mmol) and N1-[(2,4-dimethoxyphenyl)methyl]isoquinoline-1,5-diamine (971 mg, 3.14 mmol) in 1,2-dichloroethane (30 mL). The mixture was stirred at rt for 30 min then STAB (1663 mg, 7.84 mmol) was added. The mixture was stirred at rt for 24 h. The reaction was quenched with sat. NaHCO₃ (aq) (100 mL) and extracted with DCM (3×50 mL). The combined organic layers were dried (Na₂SO₄), filtered, and concentrated. The crude product was purified by flash chromatography (Silica, 0-30% MeOH in EtOAc) to afford the product (1600 mg, 67% yield).

[M+H]⁺=676.8

¹H NMR (DMSO-d6, 400 MHz) δ 1.55 (2H, d, J=3.1 Hz), 1.91 (2H, s), 3.31 (3H, s), 3.42 (2H, d, J=6.9 Hz), 3.71 (3H, s), 3.83 (3H, s), 3.95 (2H, d, J=6.1 Hz), 4.13 (2H, s), 4.59 (2H, d, J=5.6 Hz), 5.12 (2H, s), 5.76 (1H, d, J=2.7 Hz), 5.88 (1H, dd, J=7.5, 2.8 Hz), 6.01 (1H, s), 6.39 (1H, dd, J=8.4, 2.4 Hz), 6.55 (1H, d, J=2.4 Hz), 6.73 (1H, d, J=7.7 Hz), 6.88 (1H, d, J=6.1 Hz), 7.02 (1H, d, J=8.4 Hz), 7.24 (1H, t, J=8.0 Hz), 7.29-7.42 (6H, m), 7.46 (1H, d, J=8.4 Hz), 7.51 (1H, d, J=7.6 Hz), 7.71 (1H, d, J=6.1 Hz).

### 4-((1-(((1-((2,4-Dimethoxybenzyl)amino)isoquinolin-5-yl)amino)methyl)-2-azabicyclo[2.1.1]hexan-4-yl)methoxy)-1-methylpyridin-2(1H)-one

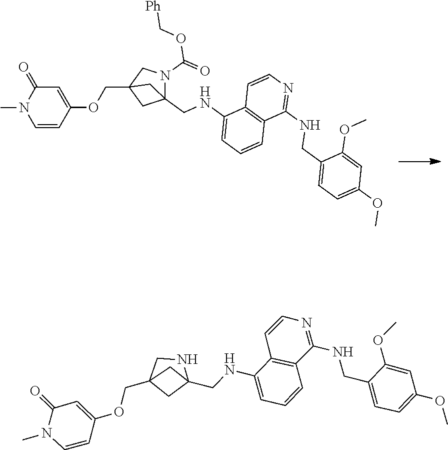

A solution of benzyl 1-[[[1-[(2,4-dimethoxyphenyl)methylamino]-5-isoquinolyl]amino]methyl]-4-[(1-methyl-2-oxo-4-pyridyl)oxymethyl]-2-azabicyclo[2.1.1]hexane-2-carboxylate (1350 mg, 2.00 mmol) in MeOH (10 mL) was added to a mixture of Pd(OH)₂/C (20 wt %) (338 mg, 0.481 mmol) in MeOH (10 mL). The mixture was subjected to hydrogenation at 1 atmosphere and rt for 66 h. The mixture was filtered over Celite washing with MeOH (3×20 mL) and the filtrate was concentrated to afford the product (1100 mg, 95% yield).

[M+H]⁺=542.4

¹H NMR (DMSO-d6, 400 MHz) δ 1.41 (2H, s), 1.65 (2H, s), 2.87 (2H, s), 3.35 (3H, s), 3.44 (2H, d, J=5.1 Hz), 3.71 (3H, s), 3.82 (3H, s), 4.14 (2H, s), 4.59 (2H, d, J=5.4 Hz), 5.77 (1H, d, J=2.7 Hz), 5.83-5.94 (2H, m), 6.39 (1H, dd, J=8.4, 2.3 Hz), 6.55 (1H, d, J=2.3 Hz), 6.68 (1H, d, J=7.9 Hz), 7.02 (1H, d, J=8.3 Hz), 7.08 (1H, d, J=6.1 Hz), 7.25 (1H, s), 7.38 (1H, s), 7.45 (1H, d, J=8.4 Hz), 7.52 (1H, d, J=7.6 Hz), 7.73 (1H, d, J=6.1 Hz), one exchangeable proton not observed.

### Intermediate 27

### 1-(Benzenesulfonyl)-4-bromo-2-chloropyrrolo[2,3-b]pyridine

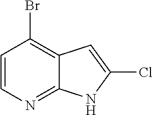 [SMILES: Clc1cc2c(Br)ccnc2[nH]1]

### 1-(Benzenesulfonyl)-4-bromopyrrolo[2,3-b]pyridine

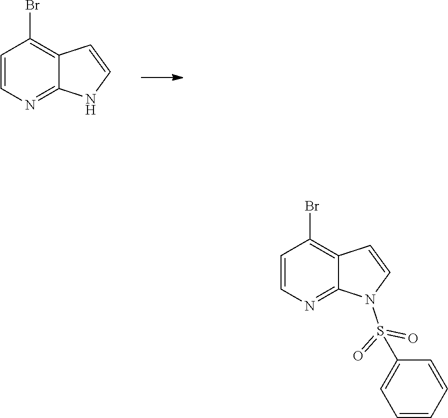 [SMILES: Brc1ccnc2[nH]ccc12 ; O=S(=O)(c1ccccc1)n1ccc2c(Br)ccnc21]

To a solution of 4-bromo-1H-pyrrolo[2,3-b]pyridine (5.00 g, 25.4 mmol) in DCM (130 mL) was added benzenesulfonyl chloride (4.86 mL, 38.1 mmol), 4-Dimethylaminopyridine (310 mg, 2.54 mmol) and TEA (10.6 mL, 76.1 mmol). The reaction mixture was stirred at room temperature for 2 h. Upon completion the reaction mixture was concentrated. The product was suspended in DCM (50 mL) and concentrated onto silica. The material was purified via flash chromatography (Silica, 0-50% EtOAc in Pet. Ether) to afford the product (8.39 g, 98% yield).

[M+H]⁺=338.9

### 1-(Benzenesulfonyl)-4-bromo-2-chloropyrrolo[2,3-b]pyridine

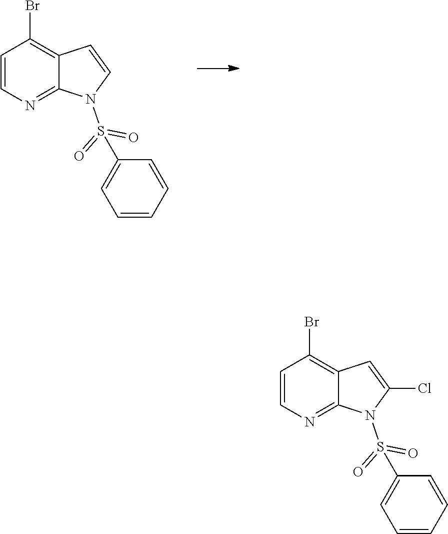 [SMILES: O=S(=O)(c1ccccc1)n1ccc2c(Br)ccnc21 ; O=S(=O)(c1ccccc1)n1c(Cl)cc2c(Br)ccnc21]

1-(Benzenesulfonyl)-4-bromopyrrolo[2,3-b]pyridine (1.00 g, 2.97 mmol) in anhydrous THF (16 mL) was cooled to −41° C. and lithium diisopropylamide (2M in THF) (356 mL, 7.12 mmol) was added slowly. The resulting precipitate was stirred for 30 minutes at −41° C. before benzenesulfonyl chloride (757 μL, 5.93 mmol) was added. The reaction mixture was stirred for 2.5 h at −41° C.

The reaction mixture was quenched with 10 ml water and diluted with 20 mL EtOAc. The layers were separated and the aq layer was back extracted with EtOAc (2×20 mL). The organic layers were combined, washed with brine (10 mL), dried (MgSO₄), filtered and concentrated. The product was purified via flash chromatography (Silica, 0-60% EtOAc in Pet. Ether) to afford the product (895 mg, 49% yield).

[M+H]⁺=372.9

### 1-(Benzenesulfonyl)-4-bromo-2-chloropyrrolo[2,3-b]pyridine

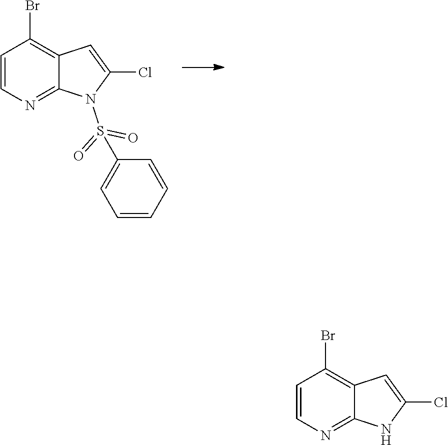 [SMILES: O=S(=O)(c1ccccc1)n1c(Cl)cc2c(Br)ccnc21 ; Clc1cc2c(Br)ccnc2[nH]1]

1-(Benzenesulfonyl)-4-bromo-2-chloropyrrolo[2,3-b]pyridine (895 mg, 1.44 mmol) was taken up in 1,4-Dioxane (7 mL) and NaOtBu (324 mg, 2.89 mmol) was added. The reaction mixture was stirred at 80° C. for 2 h, diluted with EtOAc (10 mL) and washed with brine (10 mL). Layers were separated and the organic layer was dried (MgSO₄), filtered and concentrated. The crude material was purified via flash chromatography (Silica, 0-25% EtOAc in Pet. Ether) to afford the product (295 mg, 88% yield).

[M+H]⁺=232.9

¹H NMR (CDCl₃, 400 MHz) δ 6.47 (1H, s), 7.32 (1H, d, J=5.3 Hz), 8.11 (1H, d, J=5.3 Hz) NH proton not observed

### SPECIFIC EXAMPLES OF THE PRESENT INVENTION

### Example Number 1001

### N5-((3-((Imidazo[1,2-a]pyridin-7-yloxy)methyl)bicyclo[1.1.1]pentan-1-yl)methyl)isoquinoline-1,5-diamine

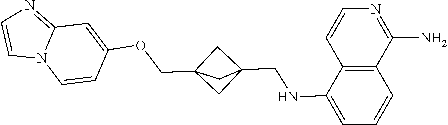 [SMILES: Nc1nccc2c(NCC34CC(COc5ccn6ccnc6c5)(C3)C4)cccc12]

### N1-[(2,4-dimethoxyphenyl)methyl]-N5-[[3-(imidazo[1,2-a]pyridin-7-yloxymethyl)-1-bicyclo[1.1.1]pentanyl]methyl]isoquinoline-1,5-diamine

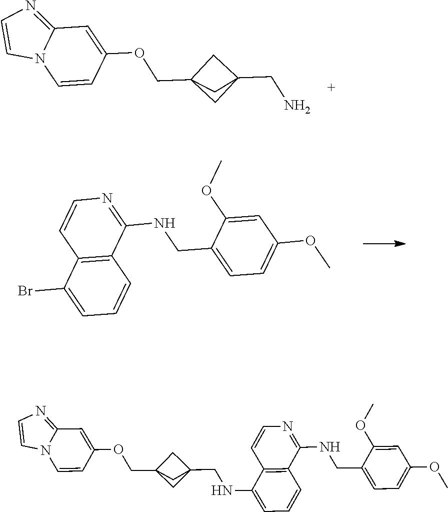 [SMILES: NCC12CC(COc3ccn4ccnc4c3)(C1)C2 ; COc1ccc(CNc2nccc3c(Br)cccc23)c(OC)c1 ; COc1ccc(CNc2nccc3c(NCC45CC(COc6ccn7ccnc7c6)(C4)C5)cccc23)c(OC)c1]

Using General Method 4, [3-(imidazo[1,2-a]pyridin-7-yloxymethyl)-1-bicyclo[1.1.1]pentanyl]methanamine (100 mg, 0.37 mmol) was reacted with 5-bromo-N-(2,4-dimethoxybenzyl)isoquinolin-1-amine (138 mg, 0.37 mmol) and NaOtBu (71 mg, 0.74 mmol) in 1,4-dioxane (5 mL) in the presence of BrettPhos Pd G3 (34 mg, 0.04 mmol) in a sealed vial at 90° C. for 6 h. The reaction was cooled and concentrated onto silica. The residue was purified by flash chromatography (Silica, 0-15% MeOH in DCM) to afford the product (70 mg, 35% yield).

[M+H]⁺=536.3

### N5-((3-((Imidazo[1,2-a]pyridin-7-yloxy)methyl)bicyclo[1.1.1]pentan-1-yl)methyl)isoquinoline-1,5-diamine (Example 1001)

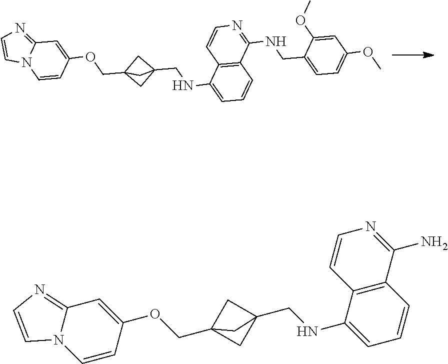 [SMILES: COc1ccc(CNc2nccc3c(NCC45CC(COc6ccn7ccnc7c6)(C4)C5)cccc23)c(OC)c1 ; Nc1nccc2c(NCC34CC(COc5ccn6ccnc6c5)(C3)C4)cccc12]

N1-[(2,4-dimethoxyphenyl)methyl]-N5-[[3-(imidazo[1,2-a]pyridin-7-yloxymethyl)-1-bicyclo[1.1.1]pentanyl]methyl]isoquinoline-1,5-diamine (70 mg, 0.13 mmol) was deprotected according to General Method 8 in TFA (1 mL, 8.63 mmol) at rt for 60 min. The solvent was removed in vacuo. The crude residue was suspended in MeOH (2 mL) and loaded on to a 2 g SCX-2 column, which was flushed with MeOH (10 mL), followed by 1N NH₃ in MeOH (10 mL) to elute the crude product. The crude product was purified by automated prep HPLC (mass directed 2-60% MeCN in water with a basic mobile phase of 0.1% NH₃ in water over 20 min) and lyophilized to afford the product (20 mg, 39% yield).

[M+H]⁺=386.2

¹H NMR (DMSO, 400 MHz) δ 1.75 (6H, s), 4.04 (2H, s), 5.87 (1H, t, J=5.9 Hz), 6.47 (2H, s), 6.56 (1H, dd, J=7.4, 2.5 Hz), 6.65 (1H, d, J=7.7 Hz), 6.87 (1H, d, J=2.5 Hz), 7.11-7.16 (1H, m), 7.19 (1H, t, J=8.0 Hz), 7.31 (1H, d, J=8.3 Hz), 7.36 (1H, d, J=1.3 Hz), 7.63-7.75 (2H, m), 8.35 (1H, dd, J=7.4, 0.6 Hz)

### Example Numbers 1002, 1002.1 and 1002.2

### N5-((3-(((5,6,7,8-Tetrahydroimidazo[1,2-a]pyridin-7-yl)oxy)methyl)bicyclo[1.1.1]pentan-1-yl)methyl)isoquinoline-1,5-diamine

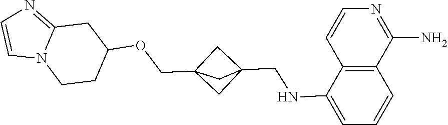 [SMILES: Nc1nccc2c(NCC34CC(COC5CCn6ccnc6C5)(C3)C4)cccc12]

### [3-(5,6,7,8-Tetrahydroimidazo[1,2-a]pyridin-7-yloxymethyl)-1-bicyclo[1.1.1]pentanyl]methanamine

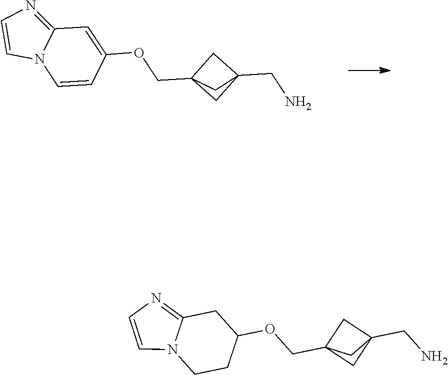 [SMILES: NCC12CC(COc3ccn4ccnc4c3)(C1)C2 ; NCC12CC(COC3CCn4ccnc4C3)(C1)C2]

[3-(imidazo[1,2-a]pyridin-7-yloxymethyl)-1-bicyclo[1.1.1]pentanyl]methanamine (100 mg, 0.41 mmol) was dissolved in EtOH (10 mL) and subjected to hydrogenation in the H-Cube® at 70° C., 50 bar, 1 mL/min using a 10% Pd/C CatCart. After 6 passes, the solvent was removed in vacuo to afford the product (70 mg, 55% yield).

[M+H]⁺=248.1

### N1-[(2,4-Dimethoxyphenyl)methyl]-N5-[[3-(5,6,7,8-tetrahydroimidazo[1,2-a]pyridin-7-yloxymethyl)-1-bicyclo[1.1.1]pentanyl]methyl]isoquinoline-1,5-diamine

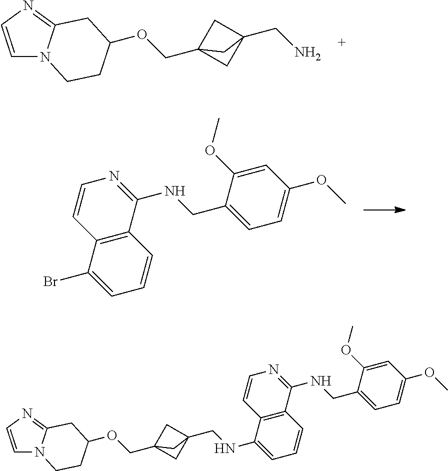 [SMILES: NCC12CC(COC3CCn4ccnc4C3)(C1)C2 ; COc1ccc(CNc2nccc3c(Br)cccc23)c(OC)c1 ; COc1ccc(CNc2nccc3c(NCC45CC(COC6CCn7ccnc7C6)(C4)C5)cccc23)c(OC)c1]

Using General Method 4, [3-(5,6,7,8-tetrahydroimidazo[1,2-a]pyridin-7-yloxymethyl)-1-bicyclo[1.1.1]pentanyl]methanamine (64 mg, 0.21 mmol) was reacted with 5-bromo-N-(2,4-dimethoxybenzyl)isoquinolin-1-amine (77 mg, 0.21 mmol) in 1,4-dioxane (5 mL) in the presence of NaOtBu (40 mg, 0.41 mmol) and BrettPhos Pd G4 (19 mg, 0.02 mmol), in a sealed vial at 90° C. for 18 h. After that time, the reaction was cooled, filtered through Celite® and the filter washed with EtOAc (50 mL) and MeOH (5 mL) and the combined filtrates concentrated in vacuo. The residue was purified by flash chromatography (Silica, 0-100% DCM in Pet. Ether followed by 0-20% MeOH in DCM) to afford the product (22 mg, 20% yield).

[M+H]⁺=540.3

### N5-((3-(((5,6,7,8-Tetrahydroimidazo[1,2-a]pyridin-7-yl)oxy)methyl)bicyclo[1.1.1]pentan-1-yl)methyl)isoquinoline-1,5-diamine (Example 1002)

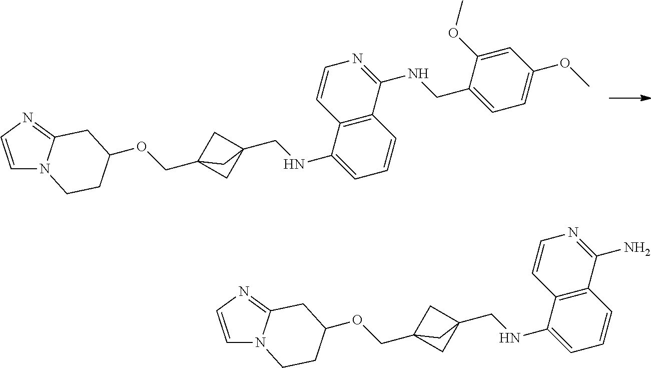 [SMILES: COc1ccc(CNc2nccc3c(NCC45CC(COC6CCn7ccnc7C6)(C4)C5)cccc23)c(OC)c1 ; Nc1nccc2c(NCC34CC(COC5CCn6ccnc6C5)(C3)C4)cccc12]

N1-[(2,4-Dimethoxyphenyl)methyl]-N5-[[3-(5,6,7,8-tetrahydroimidazo[1,2-a]pyridin-7-yloxymethyl)-1-bicyclo[1.1.1]pentanyl]methyl]isoquinoline-1,5-diamine (22 mg, 0.04 mmol) was deprotected according to General Method 8, in TFA (0.46 mL, 6.11 mmol) at rt and for 3 h. The solvent was removed in vacuo. The crude residue was suspended in MeOH (2 mL) and loaded onto a 2 g SCX-2 column, which was flushed with MeOH (10 mL), followed by 7N NH₃ in MeOH (2×10 mL) to elute the crude compound. The crude product was purified by automated prep HPLC (mass directed 2-60% MeCN in water with a basic mobile phase of 0.1% NH₃ in water over 20 min) and lyophilized to afford the product. LCMS: [M+H]⁺=390.2

¹H NMR (CDCl₃, 400 MHz) δ 1.74 (6H, s), 2.04-2.17 (2H, m), 2.96 (1H, dd, J=16.8, 5.8 Hz), 3.08 (1H, dd, J=16.7, 4.7 Hz), 3.32 (2H, d, J=3.6 Hz), 3.47-3.59 (2H, m), 3.84-3.96 (2H, m), 4.05-4.14 (1H, m), 4.22 (1H, s), 5.18 (2H, s), 6.71 (1H, dd, J=7.7, 0.8 Hz), 6.79 (1H, d, J=1.3 Hz), 6.93 (1H, dd, J=6.2, 1.0 Hz), 6.99 (1H, d, J=1.3 Hz), 7.11 (1H, dt, J=8.5, 1.0 Hz), 7.34 (1H, t, J=8.0 Hz), 7.91 (1H, d, J=6.2 Hz)

(S*)-N5-((3-(((5,6,7,8-Tetrahydroimidazo[1,2-a]pyridin-7-yl)oxy)methyl)bicyclo[1.1.1]pentan-1-yl)methyl)isoquinoline-1,5-diamine (Example 1002.1) and (R*)-N5-((3-(((5,6,7,8-tetrahydroimidazo[1,2-a]pyridin-7-yl)oxy)methyl)bicyclo[1.1.1]pentan-1-yl)methyl)isoquinoline-1,5-diamine (Example 1002.2) were prepared by chiral separation of the racemate N5-((3-(((5,6,7,8-tetrahydroimidazo[1,2-a]pyridin-7-yl)oxy)methyl)bicyclo[1.1.1]pentan-1-yl)methyl)isoquinoline-1,5-diamine (Example 1002).

### Example Number 1004

### 5-N-[[3-(6,8-Dihydro-5H-imidazo[1,2-a]pyrazin-7-ylmethyl)-1-bicyclo[1.1.1]pentanyl]methyl]isoquinoline-1,5-diamine

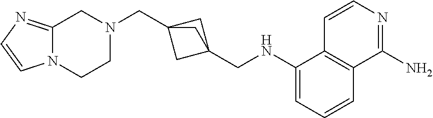 [SMILES: Nc1nccc2c(NCC34CC(CN5CCn6ccnc6C5)(C3)C4)cccc12]

### Tert-butyl N-[[3-(6,8-dihydro-5H-imidazo[1,2-a]pyrazin-7-ylmethyl)-1-bicyclo[1.1.1]pentanyl]methyl]carbamate

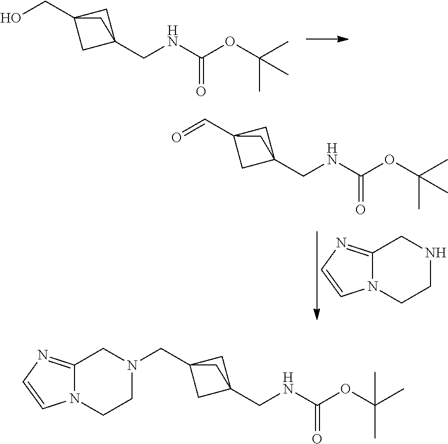

Tert-Butyl N-{[3-(hydroxymethyl)bicyclo[1.1.1]pentan-1-yl]methyl}carbamate (100 mg, 0.44 mmol) was taken up in DCM (5 mL) while cooling in an ice/water bath. Dess-Martin periodinane (261 mg, 0.62 mmol) was added and the mixture stirred at rt for 3 h. Sat. Na₂S₂O₃ (aq) (5 mL) and sat. NaHCO₃ (aq) (5 mL), were added and the mixture stirred for 15 min and extracted with DCM (3×20 mL). The combined organics were washed with brine (10 mL), dried (MgSO₄) and concentrated. The residue was suspended in THF (10 mL). 5,6,7,8-Tetrahydroimidazo[1,2-a]pyrazine (60 mg, 0.48 mmol) was added and the reaction was stirred for 15 min. STAB (278 mg, 1.32 mmol) was added and the mixture was stirred at rt for 18 h. Water (10 mL) was added and the mixture extracted with DCM (3×10 mL). The combined organics were washed with brine (10 mL), dried (MgSO₄) and concentrated. The residue was passed directly through an SCX and washed with MeOH. The product was eluted with a solution of 1M NH₃ in MeOH and concentrated to afford the product (75 mg, 51% yield).

[M+H]⁺=333.2

### [3-(6,8-Dihydro-5H-imidazo[1,2-a]pyrazin-7-ylmethyl)-1-bicyclo[1.1.1]pentanyl]methanamine

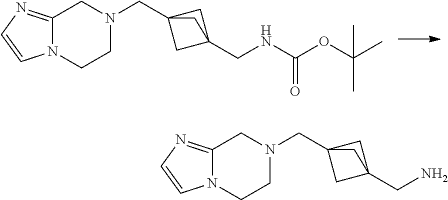

tert-Butyl N-[[3-(6,8-dihydro-5H-imidazo[1,2-a]pyrazin-7-ylmethyl)-1-bicyclo[1.1.1]pentanyl]methyl]carbamate (75 mg, 0.23 mmol) was treated with 4M HCl in dioxane according to General Method 5a, and purified according to Method (ii), to afford the product (66 mg, 99% yield).

[M+H]⁺=233.1

### 5-N-[[3-(6,8-Dihydro-5H-imidazo[1,2-a]pyrazin-7-ylmethyl)-1-bicyclo[1.1.1]pentanyl]methyl]-1-N-[(2,4-dimethoxyphenyl)methyl]isoquinoline-1,5-diamine

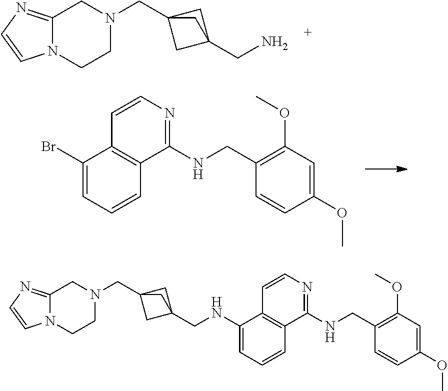 [SMILES: NCC12CC(CN3CCn4ccnc4C3)(C1)C2 ; COc1ccc(CNc2nccc3c(Br)cccc23)c(OC)c1 ; COc1ccc(CNc2nccc3c(NCC45CC(CN6CCn7ccnc7C6)(C4)C5)cccc23)c(OC)c1]

5-Bromo-N-[(2,4-dimethoxyphenyl)methyl]isoquinolin-1-amine (106 mg, 0.28 mmol) and [3-(6,8-dihydro-5H-imidazo[1,2-a]pyrazin-7-ylmethyl)-1-bicyclo[1.1.1]pentanyl]methanamine (66 mg, 0.28 mmol) were reacted according to General Method 4 in the presence of Brettphos Pd G4 and Cs₂CO₃. Flash chromatography (Silica, 0-20% (10% NH₄OH in MeOH) in EtOAc) afforded the product (53 mg, 36%.

[M+H]⁺=525.3

### 5-N-[[3-(6,8-Dihydro-5H-imidazo[1,2-a]pyrazin-7-ylmethyl)-1-bicyclo[1.1.1]pentanyl]methyl]isoquinoline-1,5-diamine (Example 1004)

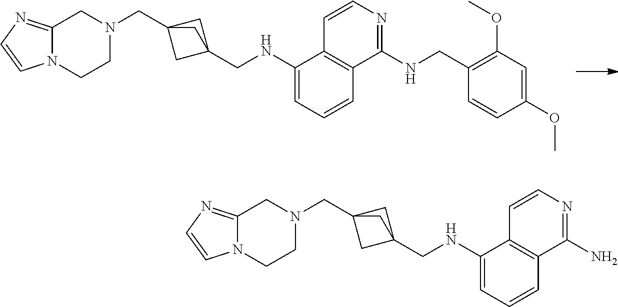

5-N-[[3-(6,8-Dihydro-5H-imidazo[1,2-a]pyrazin-7-ylmethyl)-1-bicyclo[1.1.1]pentanyl]methyl]-1-N-[(2,4-dimethoxyphenyl)methyl]isoquinoline-1,5-diamine (53 mg, 0.1 mmol) was deprotected using General Method 8. The mixture was cooled to rt and the solvent was removed in vacuo, azeotroping with toluene. The residue was dissolved in MeOH (3 mL) and passed directly through SCX-2 and flushed with MeOH (3×5 mL). The product was eluted with a solution of 1M NH₃ in MeOH (3×5 mL) and the solvent was removed in vacuo. The product was purified by flash chromatography (Silica, 0-20% (10% NH₄OH in MeOH) in DCM) and further purified by automated prep HPLC (mass directed 2-60% MeCN in water with a basic mobile phase of 0.1% NH₃ in water over 20 min) and lyophilised to afford the product (15 mg, 40% yield).

[M+H]⁺=375.2

¹H NMR (DMSO-d6, 400 MHz) δ 1.69 (6H, s), 2.56 (2H, s), 2.80 (2H, dd, J=6.3, 4.7 Hz), 3.28 (2H, d, J=5.6 Hz), 3.56 (2H, s), 3.91 (2H, t, J=5.5 Hz), 5.81 (1H, t, J=5.9 Hz), 6.47 (2H, s), 6.63 (1H, d, J=7.6 Hz), 6.80 (1H, d, J=1.2 Hz), 7.00 (1H, d, J=1.2 Hz), 7.13 (1H, d, J=6.1 Hz), 7.19 (1H, t, J=8.0 Hz), 7.30 (1H, d, J=8.3 Hz), 7.70 (1H, d, J=6.1 Hz) ppm.

### Example Number 1005.1

### N5-((3-(((1S,4S)-5-Isopropyl-2,5-diazabicyclo[2.2.1]heptan-2-yl)methyl)bicyclo[1.1.1]pentan-1-yl)methyl)isoquinoline-1,5-diamine

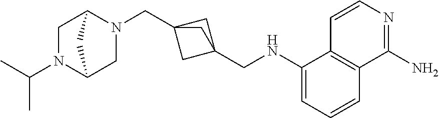 [SMILES: CC(C)N1C[C@@H]2C[C@H]1CN2CC12CC(CNc3cccc4c(N)nccc34)(C1)C2]

### Tert-butyl N-[[3-[[(15,4S)-5-propan-2-yl-2,5-diazabicyclo[2.2.1]heptan-2-yl]methyl]-1-bicyclo[1.1.1]pentanyl]methyl]carbamate

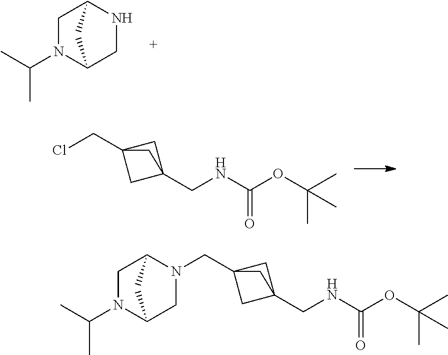

Following General Method 2b, (1S,4S)-2-(propan-2-yl)-2,5-diazabicyclo[2.2.1]heptane (51 mg, 0.37 mmol) was reacted with tert-butyl N-[[3-(chloromethyl)-1-bicyclo[1.1.1]pentanyl]methyl]carbamate (90 mg, 0.37 mmol) and K₂CO₃ (103 mg, 0.73 mmol) in MeCN (5 mL) at 60° C. for 72 h. The reaction was diluted with water (20 mL) and the aqueous was extracted with DCM (3×20 mL). The combined organics were washed with brine (20 mL), dried (MgSO₄), filtered and concentrated to afford the product (128 mg, 100% yield).

[M+H]⁺=350.1

### [3-[[(1,4S)-5-propan-2-yl-2,5-diazabicyclo[2.2.1]heptan-2-yl]methyl]-1-bicyclo[1.1.1]pentanyl]methanamine

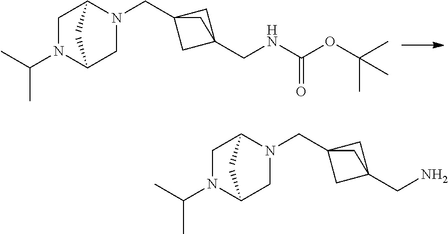

tert-Butyl N-[[3-[[(1S,4S)-5-propan-2-yl-2,5-diazabicyclo[2.2.1]heptan-2-yl]methyl]-1-bicyclo[1.1.1]pentanyl]methyl]carbamate (128 mg, 0.37 mmol) was deprotected according to General Method 5a, in 1,4-dioxane (4 mL) with 4M HCl in dioxane (916 μL, 3.66 mmol). The reaction was stirred at rt for 20 h and then concentrated. The free base of the resulting HCl salt was liberated by dissolving in MeOH and passing through PL-HCO₃ MP SPE (Agilent). The solvent was removed in vacuo to afford the product (91 mg, 100% yield).

[M+H]⁺=250.2

### 1-N-[(2,4-dimethoxyphenyl)methyl]-5-N-[[3-[[(15,4S)-5-propan-2-yl-2,5-diazabicyclo[2.2.1]heptan-2-yl]methyl]-1-bicyclo[1.1.1]pentanyl]methyl]isoquinoline-1,5-diamine

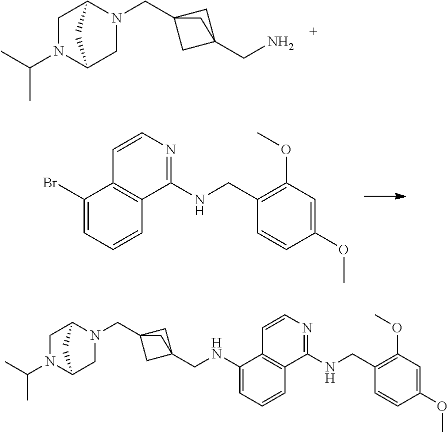

Using General Method 4, 5-bromo-N-[(2,4-dimethoxyphenyl)methyl]isoquinolin-1-amine (136 mg, 0.36 mmol) was reacted with [3-[[(1S,4S)-5-propan-2-yl-2,5-diazabicyclo[2.2.1]heptan-2-yl]methyl]-1-bicyclo[1.1.1]pentanyl]methanamine (91 mg, 0.36 mmol) in the presence of Cs₂CO₃ (239 mg, 0.73 mmol) and Brettphos Pd G4 (33 mg, 0.04 mmol) in 1,4-dioxane (5 mL) in a sealed vial at 60° C. for 72 h. The reaction was cooled to rt and filtered through Celite®. The crude product was purified by flash chromatography (SNAP KP-NH (Biotage®), 0-20% MeOH in DCM) to afford the product (100 mg, 51% yield).

[M+H]⁺=542.4

### N5-((3-(((15,4S)-5-isopropyl-2,5-diazabicyclo[2.2.1]heptan-2-yl)methyl)bicyclo[1.1.1]pentan-1-yl)methyl)isoquinoline-1,5-diamine (Example 1005.1)

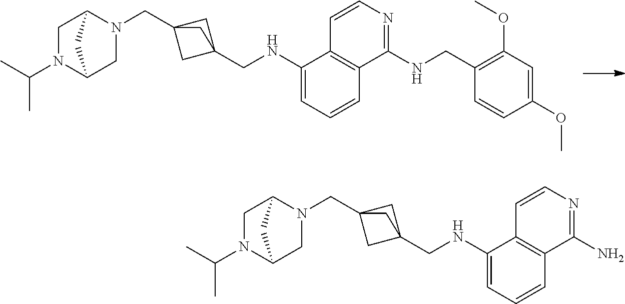

1-N-[(2,4-Dimethoxyphenyl)methyl]-5-N-[[3-[[(1S,4S)-5-propan-2-yl-2,5-diazabicyclo[2.2.1]heptan-2-yl]methyl]-1-bicyclo[1.1.1]pentanyl]methyl]isoquinoline-1,5-diamine (101 mg, 0.19 mmol) was deprotected using General Method 8. The reaction mixture was cooled to rt and the solvent was removed, azeoptroping with toluene (10 mL). The crude product was purified by SCX, followed by automated prep HPLC (mass directed 2-60% MeCN in water with a basic mobile phase of 0.1% NH₃ in water over 20 min) and lyophillised to afford the product (34 mg, 47% yield).

[M+H]⁺=392.2

¹H NMR (DMSO-d6, 400 MHz) δ 0.89 (3H, d, J=6.1 Hz), 0.94 (3H, d, J=6.0 Hz), 1.45 (1H, d, J=9.0 Hz), 1.51 (1H, d, J=9.0 Hz), 1.58 (6H, s), 2.36-2.40 (1H, m), 2.41 (1H, d, J=7.9 Hz), 2.46-2.49 (1H, m), 2.51-2.53 (2H, m), 2.55 (2H, d, J=9.9 Hz), 2.68 (1H, dd, J=9.3, 2.6 Hz), 3.13 (1H, s), 3.27 (2H, d, J=5.7 Hz), 5.80 (1H, t, J=5.8 Hz), 6.48 (2H, s), 6.61 (1H, d, J=7.7 Hz), 7.09-7.15 (1H, m), 7.18 (1H, t, J=8.0 Hz), 7.30 (1H, d, J=8.3 Hz), 7.70 (1H, d, J=6.0 Hz)

### Example Number 1006

### N5-((3-(((2-Methylimidazo[1,2-a]pyridin-7-yl)oxy)methyl)bicyclo[1.1.1]pentan-1-yl)methyl)isoquinoline-1,5-diamine

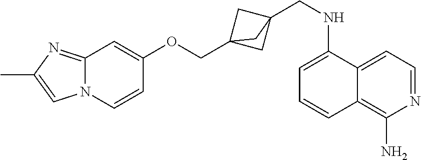 [SMILES: Cc1cn2ccc(OCC34CC(CNc5cccc6c(N)nccc56)(C3)C4)cc2n1]

### 1-N-[(2,4-Dimethoxyphenyl)methyl]-5-N-[[3-[(2-methylimidazo[1,2-a]pyridin-7-yl)oxymethyl]-1-bicyclo[1.1.1]pentanyl]methyl]isoquinoline-1,5-diamine

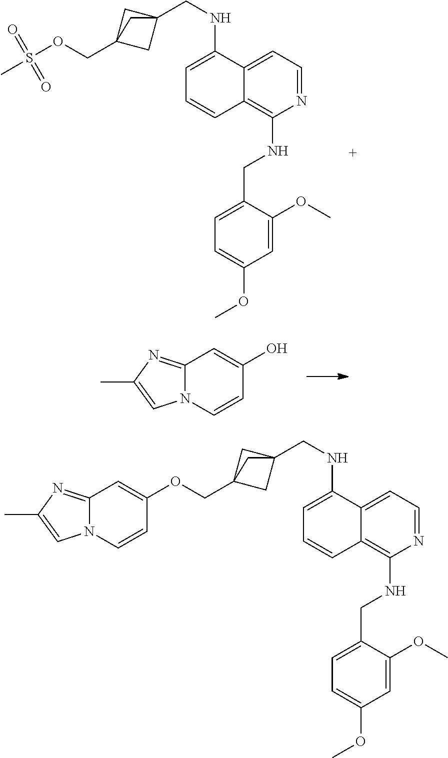

Following General Method 2b [3-[[[1-[(2,4-dimethoxyphenyl)methylamino]isoquinolin-5-yl]amino]methyl]-1-bicyclo[1.1.1]pentanyl]methyl methanesulfonate (120 mg, 0.24 mmol) was reacted with 2-methylimidazo[1,2-a]pyridin-7-ol (39 mg, 0.26 mmol) and K₂CO₃ (146 mg, 1.05 mmol) in MeCN (3 mL) at 60° C. for 72 h. The mixture was diluted with water (5 ml). The aqueous was extracted with DCM (3×10 mL). The combined organics were washed with brine, dried (MgSO₄) and concentrated. Flash chromatography (Silica, 0-20% (10% NH₄OH in MeOH) in DCM) afforded the product (31 mg, 23% yield).

[M+H]⁺=550.1

### N5-((3-(((2-Methylimidazo[1,2-a]pyridin-7-yl)oxy)methyl)bicycle[1.1.1]pentan-1-yl)methyl)isoquinoline-1,5-diamine (Example 1006)

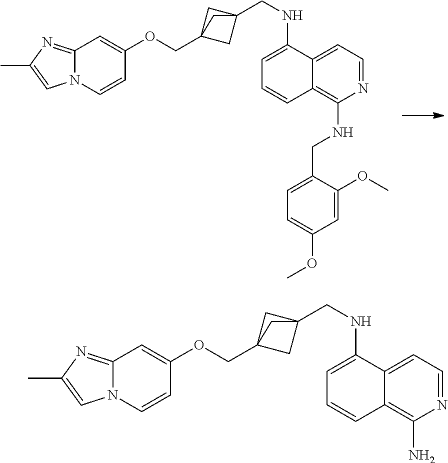

1-N-[(2,4-Dimethoxyphenyl)methyl]-5-N-[[3-[(2-methylimidazo[1,2-a]pyridin-7-yl)oxymethyl]-1-bicyclo[1.1.1]pentanyl]methyl]isoquinoline-1,5-diamine (31 mg, 0.06 mmol) was deprotected according to General Method 8, in TFA (6 mg, 0.06 mmol) at 50° C. for 30 min. The solvent was removed in vacuo, azeoptroping with toluene (20 mL). The residue was dissolved in MeOH (3 mL), filtered and passed through SCX-2 and washed with MeOH (3×5 mL). The required compound was eluted with a solution of 1M NH₃ in MeOH (3×5 mL) and the solvent was removed. Flash chromatography (Silica, 0-20% (10% NH₄OH in MeOH) in DCM) afforded the product (6 mg, 25% yield).

[M+H]⁺=400.2

¹H NMR (CD₃CN, 400 MHz) δ 1.83 (6H, s), 2.31 (3H, d, J=0.9 Hz), 3.40 (2H, d, J=4.1 Hz), 4.06 (2H, s), 4.86 (1H, s), 5.57 (2H, s), 6.46 (1H, dd, J=7.4, 2.5 Hz), 6.72 (1H, d, J=2.5 Hz), 6.77-6.81 (1H, m), 7.05 (1H, dd, J=6.1, 1.0 Hz), 7.20 (1H, dt, J=8.3, 1.0 Hz), 7.30 (1H, s), 7.34 (1H, t, J=8.0 Hz), 7.83 (1H, d, J=6.2 Hz), 8.03 (1H, dd, J=7.4, 0.7 Hz) ppm.

### Example Number 1012

### N-((3-((Imidazo[1,2-a]pyridin-7-yloxy)methyl)bicyclo[1.1.1]pentan-1-yl)methyl)-2-methyl-1H-pyrrolo[2,3-b]pyridin-4-amine

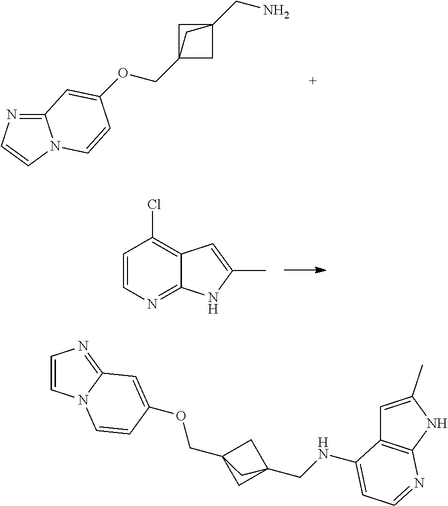 [SMILES: NCC12CC(COc3ccn4ccnc4c3)(C1)C2 ; Cc1cc2c(Cl)ccnc2[nH]1 ; Cc1cc2c(NCC34CC(COc5ccn6ccnc6c5)(C3)C4)ccnc2[nH]1]

According to General Method 4, (3-((imidazo[1,2-a]pyridin-7-yloxy)methyl)bicyclo[1.1.1]pentan-1-yl)methanamine (233 μmol) and 4-chloro-2-methyl-1H-pyrrolo[2,3-b]pyridine (228 μmol) were reacted in the presence of BrettPhos Pd G3 (11.4 μmol) and LiHMDS (547 μmol). The reaction was quenched with AcOH. The crude product was purified by an SCX eluting with NH₃ in MeOH followed by purification by flash chromatography (Silica, 0-6% (0.7M NH₃ in MeOH) in DCM) then re-purified by flash chromatography (Silica, 0-9% (0.7M NH₃ in MeOH) in EtOAc). Product was further purified by automated prep HPLC (mass directed MeCN 15-45% in water over 12.5 min in basic mobile phase) to obtain the product (18 mg, 21%).

[M+H]=374.3

¹H NMR (500 MHz, DMSO-d6) δ 1.72 (6H, s), 2.29 (3H, s), 3.32 (2H, s, obscured by H2O), 4.03 (2H, s), 6.08 (1H, d, J=5.5 Hz), 6.16-6.26 (2H, m), 6.56 (1H, dd, J=7.4, 2.5 Hz), 6.86 (1H, d, J=2.5 Hz), 7.36 (1H, s), 7.67 (1H, d, J=5.5 Hz), 7.71 (1H, s), 8.35 (1H, d, J=7.4 Hz), 10.89 (1H, s).

### Example Number 1013

### 5-[[3-(Imidazo[1,2-a]pyridin-7-yloxymethyl)-1-bicyclo[1.1.1]pentanyl]methoxy]isoquinolin-1-amine

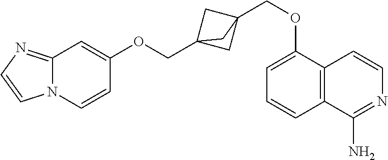 [SMILES: Nc1nccc2c(OCC34CC(COc5ccn6ccnc6c5)(C3)C4)cccc12]

### Methyl 3-[[1-[(2,4-dimethoxyphenyl)methylamino]-5-isoquinolyl]oxymethyl]bicyclo[1.1.1]pentane-1-carboxylate

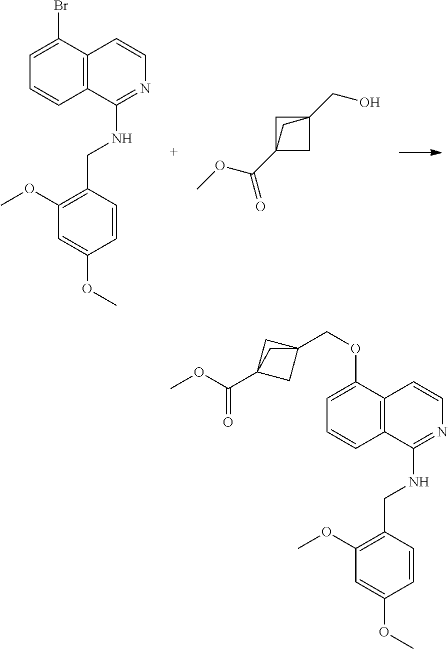 [SMILES: COc1ccc(CNc2nccc3c(Br)cccc23)c(OC)c1 ; COC(=O)C12CC(CO)(C1)C2 ; COC(=O)C12CC(COc3cccc4c(NCc5ccc(OC)cc5OC)nccc34)(C1)C2]

According to General Method 4, 5-bromo-N-[(2,4-dimethoxyphenyl)methyl]isoquinolin-1-amine (478 mg, 1.28 mmol) and methyl 3-(hydroxymethyl)bicyclo[1.1.1]pentane-1-carboxylate (200 mg, 1.28 mmol) were reacted in the presence of CS₂CO₃ (2099 mg, 6.4 mmol) and Brettphos Pd G4 (147 mg, 0.16 mmol) in 1,4-dioxane (10 mL) at 90° C. in a sealed vial for 3 days. Purification by flash chromatography (silica, 0-80% EtOAc in Pet. Ether) afforded the product (230 mg, 40% yield).

[M+H]⁺=449.2

### [3-[[1-[(2,4-Dimethoxyphenyl)methylamino]isoquinolin-5-yl]oxymethyl]-1-bicyclo[1.1.1]pentanyl]methanol

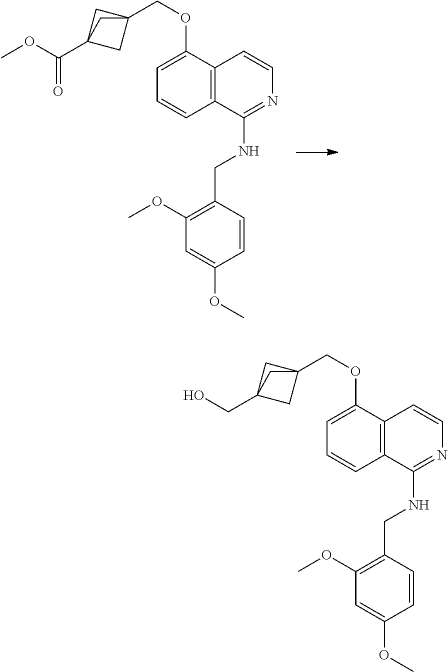 [SMILES: COC(=O)C12CC(COc3cccc4c(NCc5ccc(OC)cc5OC)nccc34)(C1)C2 ; COc1ccc(CNc2nccc3c(OCC45CC(CO)(C4)C5)cccc23)c(OC)c1]

According to General Method 3b, methyl 3-[[1-[(2,4-dimethoxyphenyl)methylamino]isoquinolin-5-yl]oxymethyl]bicyclo[1.1.1]pentane-1-carboxylate (230 mg, 0.51 mmol) was reacted with LiAlH₄ to afford the product (215 mg, 100% yield).

[M+H]⁺=421.2

### [3-[[1-[(2,4-Dimethoxyphenyl)methylamino]isoquinolin-5-yl]oxymethyl]-1-bicyclo[1.1.1]pentanyl]methyl methanesulfonate

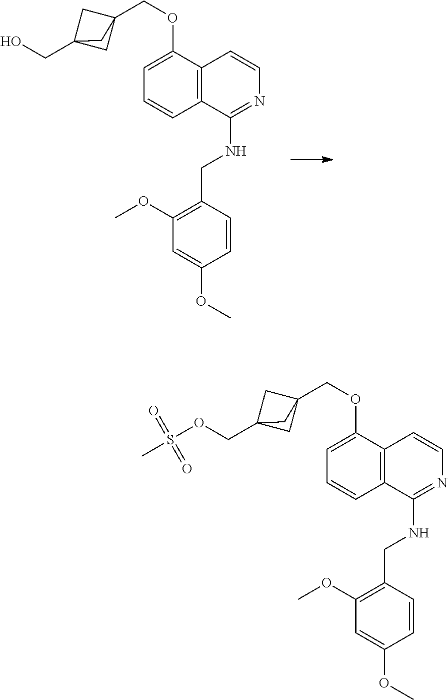 [SMILES: COc1ccc(CNc2nccc3c(OCC45CC(CO)(C4)C5)cccc23)c(OC)c1 ; COc1ccc(CNc2nccc3c(OCC45CC(COS(C)(=O)=O)(C4)C5)cccc23)c(OC)c1]

According to General Method 1a, [3-[[1-[(2,4-dimethoxyphenyl)methylamino]isoquinolin-5-yl]oxymethyl]-1-bicyclo[1.1.1]pentanyl]methanol (105 mg, 0.25 mmol) was reacted with methane sulfonyl chloride (23 UL, 0.3 mmol) to obtain the product (120 mg, 87% yield) which was used without purification.

[M+H]⁺*=499.2

### N-[(2,4-Dimethoxyphenyl)methyl]-5-[[3-(imidazo[1,2-a]pyridin-7-yloxymethyl)-1-bicyclo[1.1.1]pentanyl]methoxy]isoquinolin-1-amine

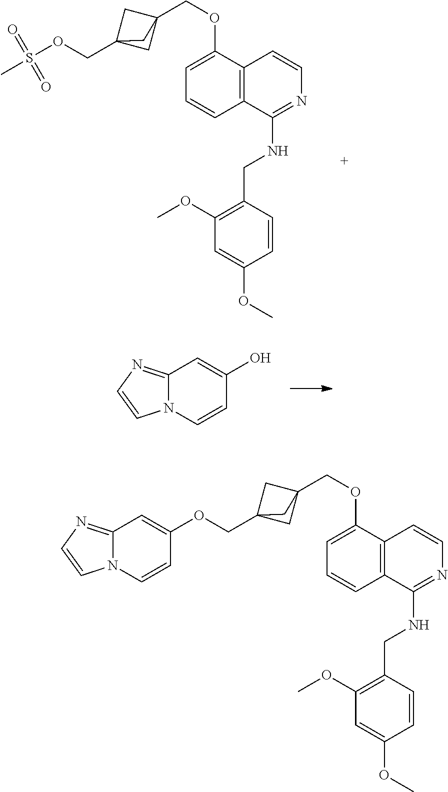

According to General Method 2a, imidazo[1,2-a]pyridin-7-ol hydrochloride (37 mg, 0.22 mmol) was reacted with [3-[[1-[(2,4-dimethoxyphenyl)methylamino]isoquinolin-5-yl]oxymethyl]-1-bicyclo[1.1.1]pentanyl]methyl methanesulfonate (120 mg, 0.22 mmol). Purification by flash chromatography (silica, 0-100% (3:3:1 EtOAc:MeCN:EtOH+2% NH₄OH) in Pet. Ether) afforded the product (23 mg, 18% yield).

[M+H]⁺=537

### 5-[[3-(Imidazo[1,2-a]pyridin-7-yloxymethyl)-1-bicyclo[1.1.1]pentanyl]methoxy]isoquinolin-1-amine

### Example 1013

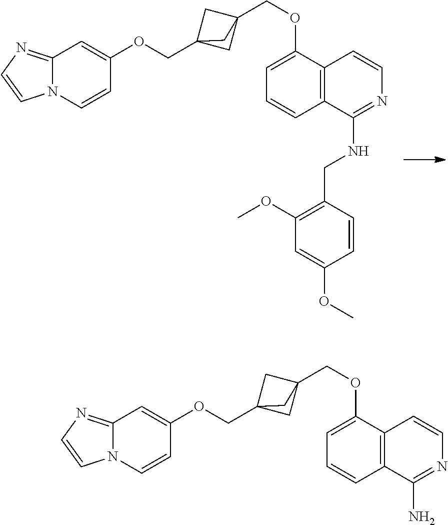 [SMILES: COc1ccc(CNc2nccc3c(OCC45CC(COc6ccn7ccnc7c6)(C4)C5)cccc23)c(OC)c1 ; Nc1nccc2c(OCC34CC(COc5ccn6ccnc6c5)(C3)C4)cccc12]

N-[(2,4-Dimethoxyphenyl)methyl]-5-[[3-(imidazo[1,2-a]pyridin-7-yloxymethyl)-1-bicyclo[1.1.1]pentanyl]methoxy]isoquinolin-1-amine (23 mg, 0.04 mmol) was reacted according to General Method 8 and purified by automated prep HPLC (mass directed 2-60% MeCN in water with a basic mobile phase of 0.1% NH₃ in water over 20 min) and lyophilized to afford the product (8 mg, 48% yield).

[M+H]⁺=387.2

¹H NMR (DMSO-d₆, 400 MHz) δ 1.88 (6H, s), 4.12 (2H, s), 4.16 (2H, s), 6.63 (1H, dd, J=7.4, 2.4 Hz), 6.76 (2H, s), 6.92 (1H, d, J=2.3 Hz), 7.07 (1H, d, J=7.7 Hz), 7.13 (1H, d, J=5.8 Hz), 7.35 (1H, t, J=8.1 Hz), 7.38 (1H, d, J=0.6 Hz), 7.70-7.74 (2H, m), 7.80 (1H, d, J=5.9 Hz), 8.38 (1H, d, J=7.4 Hz

### Example Number 1014

### 5-[2-[3-Imidazo[1,2-a]pyridin-7-yloxymethyl)-1-bicyclo[1.1.1]pentanyl]ethyl]isoquinolin-1-amine

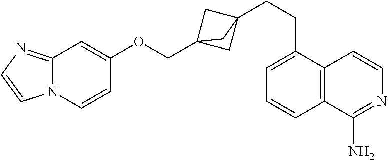 [SMILES: Nc1nccc2c(CCC34CC(COc5ccn6ccnc6c5)(C3)C4)cccc12]

### Methyl 3-[2-[1-[(2,4-dimethoxyphenyl)methylamino]isoquinolin-5-yl]ethynyl]bicyclo[1.1.1]pentane-1-carboxylate

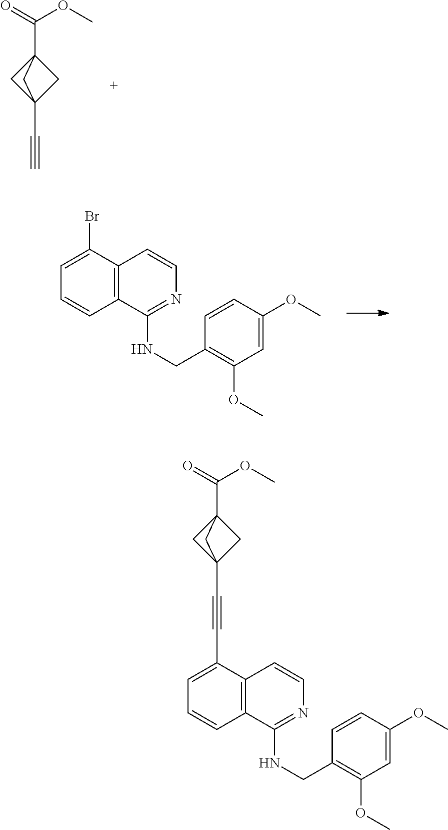 [SMILES: C#CC12CC(C(=O)OC)(C1)C2 ; COc1ccc(CNc2nccc3c(Br)cccc23)c(OC)c1 ; COC(=O)C12CC(C#Cc3cccc4c(NCc5ccc(OC)cc5OC)nccc34)(C1)C2]

To a sealed vial which had been flushed with N₂ was added 5-bromo-N-[(2,4-dimethoxyphenyl)methyl]isoquinolin-1-amine (255 mg, 0.68 mmol), CuI (38 mg, 0.2 mmol) and Pd(PPh₃)₄ (77 mg, 0.07 mmol). TEA (186 μL, 1.33 mmol) and 1,4-dioxane (5 mL) were added and the mixture was purged with N₂ while stirring for 10 min. Methyl 3-ethynylbicyclo[1.1.1]pentane-1-carboxylate (100 mg, 0.67 mmol) was added and the mixture was stirred at 50° C. for 2 days. The mixture was filtered through Celite®, washed with EtOAc and concentrated. Flash chromatography (Silica, 0-60% EtOAc in Pet. Ether) afforded the product (182 mg, 62% yield).

[M+H]⁺=443.2

### Methyl 3-[2-[1-[(2,4-dimethoxyphenyl)methylamino]isoquinolin-5-yl]ethyl]bicyclo[1.1.1]pentane-1-carboxylate

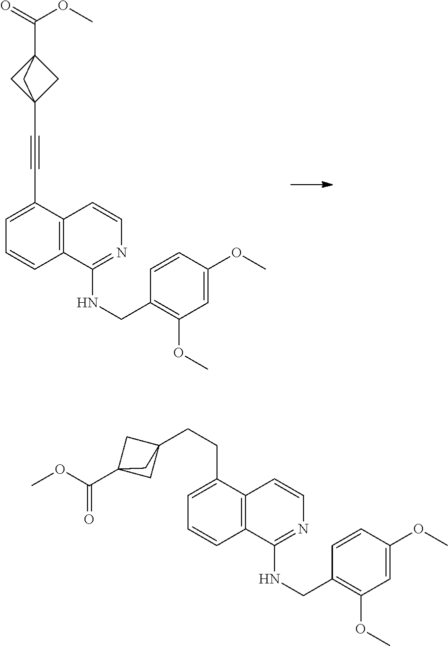

Reduction of methyl 3-[2-[1-[(2,4-dimethoxyphenyl)methylamino]isoquinolin-5-yl]ethynyl]bicyclo[1.1.1]pentane-1-carboxylate (182 mg, 0.41 mmol) was performed using General Method 3a in a solution of ethanol (20 mL) at rt, 1 bar and a flow rate of 1 mL/min using a 10% Pd/C CatCart. The solvent was removed to afford the product (166 mg, 90% yield).

[M+H]⁺=447.2

### [3-[2-[1-[(2,4-Dimethoxyphenyl)methylamino]isoquinolin-5-yl]ethyl]-1-bicyclo[1.1.1]pentanyl]methanol

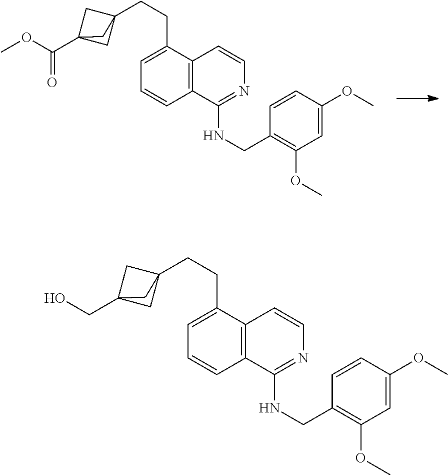

Using General Method 3b, reduction of methyl 3-[2-[1-[(2,4-dimethoxyphenyl)methylamino]isoquinolin-5-yl]ethyl]bicyclo[1.1.1]pentane-1-carboxylate (166 mg, 0.37 mmol) was carried out over 1 hour. The reaction was diluted with Et₂O and cooled in an ice/water bath before water (0.1 mL) was added, followed by 2M NaOH (aq) (0.1 mL), and water (0.3 mL). The mixture was stirred at rt for 15 min before adding Na₂SO₄. The mixture was stirred for a further 15 min, filtered and concentrated to afford the product (154 mg, 99% yield).

[M+H]⁺=419.2

### [3-[2-[1-[(2,4-Dimethoxyphenyl)methylamino]isoquinolin-5-yl]ethyl]-1-bicyclo[1.1.1]pentanyl]methyl methanesulfonate

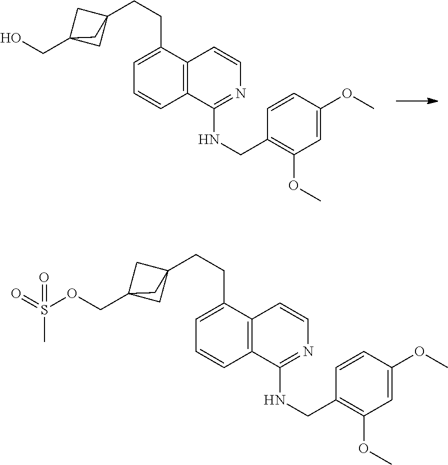 [SMILES: COc1ccc(CNc2nccc3c(CCC45CC(CO)(C4)C5)cccc23)c(OC)c1 ; COc1ccc(CNc2nccc3c(CCC45CC(COS(C)(=O)=O)(C4)C5)cccc23)c(OC)c1]

Following General Method 1a, [3-[2-[1-[(2,4-dimethoxyphenyl)methylamino]isoquinolin-5-yl]ethyl]-1-bicyclo[1.1.1]pentanyl]methanol (154 mg, 0.37 mmol) was reacted with methane sulfonyl chloride (34 μL, 0.44 mmol) while cooling in an ice/water bath for 90 min to afford the product (183 mg, 100% yield) which was used without purification by flash chromatography.

[M+H]⁺=497.2

### N-[(2,4-Dimethoxyphenyl)methyl]-5-[2-[3-(imidazo[1,2-a]pyridin-7-yloxymethyl)-1-bicyclo[1.1.1]pentanyl]ethyl]isoquinolin-1-amine

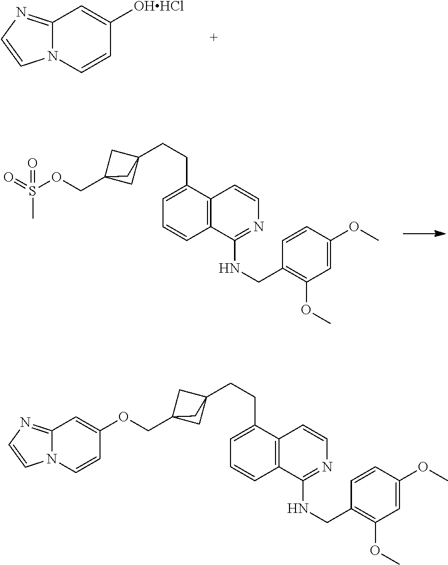

3-[2-[1-[(2,4-Dimethoxyphenyl)methylamino]isoquinolin-5-yl]ethyl]-1-bicyclo[1.1.1]pentanyl]methyl methanesulfonate (100 mg, 0.2 mmol) and imidazo[1,2-a]pyridin-7-ol hydrochloride (34 mg, 0.2 mmol) were taken up in DMF (5 mL) and TEA (28 μL, 0.2 mmol). The mixture was cooled in an ice/water bath and NaH (60% dispersion in mineral oil) (16 mg, 0.4 mmol) was added. The reaction mixture was heated to 50° C. overnight. DCM (30 mL) and water (10 mL) were added. The layers were separated and the aqueous layer was back-extracted with DCM (3×10 mL). The organic layers were combined, dried (MgSO₄) and concentrated. Flash chromatography (silica, 0-100% EtOAc in Pet. Ether followed by 0-20% MeOH in DCM) afforded the product (29 mg, 27% yield).

[M+H]⁺=535.3

### 5-[2-[3-(Imidazo[1,2-a]pyridin-7-yloxymethyl)-1-bicyclo[1.1.1]pentanyl]ethyl]isoquinolin-1-amine (Example 1014)

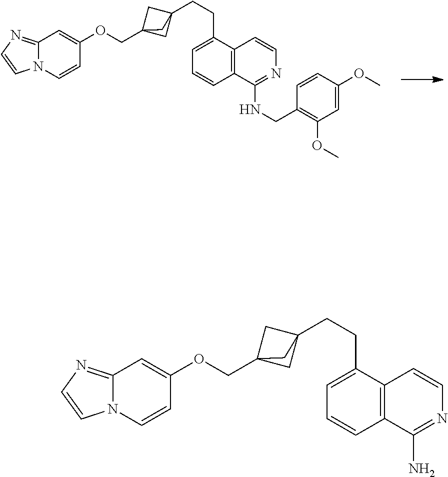 [SMILES: COc1ccc(CNc2nccc3c(CCC45CC(COc6ccn7ccnc7c6)(C4)C5)cccc23)c(OC)c1 ; Nc1nccc2c(CCC34CC(COc5ccn6ccnc6c5)(C3)C4)cccc12]

N-[(2,4-Dimethoxyphenyl)methyl]-5-[2-[3-(imidazo[1,2-a]pyridin-7-yloxymethyl)-1-bicyclo[1.1.1]pentanyl]ethyl]isoquinolin-1-amine (29 mg, 0.05 mmol) was reacted according to General Method 8 and purified by automated prep HPLC (mass directed 2-60% MeCN in water with a basic mobile phase of 0.1% NH₃ in water over 20 min) and lyophilised to afford the product (29 mg, 26% yield). Analysis indicated the presence of alkene side product carried over from the palladium catalysed hydrogenation of the alkyne. This mixture (14 mg, 0.04 mmol) was taken up in EtOH (5 ml) and subjected to hydrogenation in the H-Cube® using 10% Pd/C at rt, 1 bar, flow rate 1 ml/min. The mixture was concentrated and lyophilised to afford the product (7 mg, 50% yield).

[M+H]⁺=385.2

¹H NMR (CDCl₃, 400 MHz) δ 1.78 (6H, s), 1.86-1.97 (2H, m), 2.90-2.99 (2H, m), 4.02 (2H, s), 5.28 (2H, d, J=18.4 Hz), 6.53 (1H, dd, J=7.4, 2.4 Hz), 6.84 (1H, d, J=2.5 Hz), 7.15 (1H, dd, J=6.2, 1.0 Hz), 7.36-7.45 (2H, m), 7.48 (2H, dd, J=7.2, 1.3 Hz), 7.68 (1H, dt, J=8.3, 1.3 Hz), 7.93 (1H, dd, J=7.4, 0.7 Hz), 7.96 (1H, d, J=6.2 Hz)

### Example Number 1016

### [3-[[(1-Aminoisoquinolin-5-yl)amino]methyl]-1-bicyclo[1.1.1]pentanyl]-(6,8-dihydro-5H-imidazo[1,2-a]pyrazin-7-yl)methanone

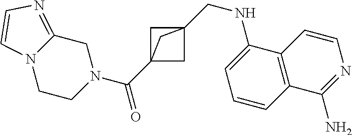 [SMILES: Nc1nccc2c(NCC34CC(C(=O)N5CCn6ccnc6C5)(C3)C4)cccc12]

### 3-[[[1-[(2,4-Dimethoxyphenyl)methylamino]isoquinolin-5-yl]amino]methyl]bicyclo[1.1.1]pentane-1-carboxylic acid

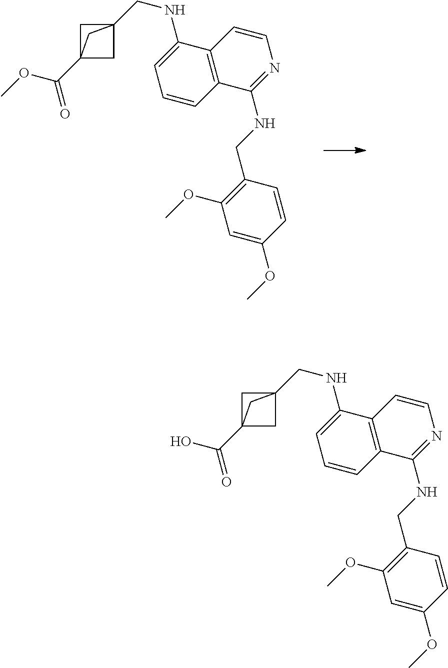 [SMILES: COC(=O)C12CC(CNc3cccc4c(NCc5ccc(OC)cc5OC)nccc34)(C1)C2 ; COc1ccc(CNc2nccc3c(NCC45CC(C(=O)O)(C4)C5)cccc23)c(OC)c1]

To a solution of methyl 3-[[[1-[(2,4-dimethoxyphenyl)methylamino]isoquinolin-5-yl]amino]methyl]bicyclo[1.1.1]pentane-1-carboxylate (100 mg, 0.22 mmol) in THF (4 mL) and water (1 mL) was added lithium hydroxide monohydrate (64 mg, 1.11 mmol) and the reaction stirred at 50° C. for 3 h.

The mixture was cooled to rt and concentrated. The crude residue was partitioned between water and CHCl₃. The aqueous layer was acidified to pH 1 with 2N HCl (aq) and the product extracted into isopropanol:CHCl₃ (1:3) (5×10 mL). The combined organic layers from the acid work-up were dried (MgSO₄), filtered and concentrated to afford the product (80 mg, 83% yield).

[M+H]⁺=434.2

### 6,8-Dihydro-5H-imidazo[1,2-a]pyrazin-7-yl-[3-[[[1-[(2,4-dimethoxyphenyl)methylamino]isoquinolin-5-yl]amino]methyl]-1-bicyclo[1.1.1]pentanyl]methanone

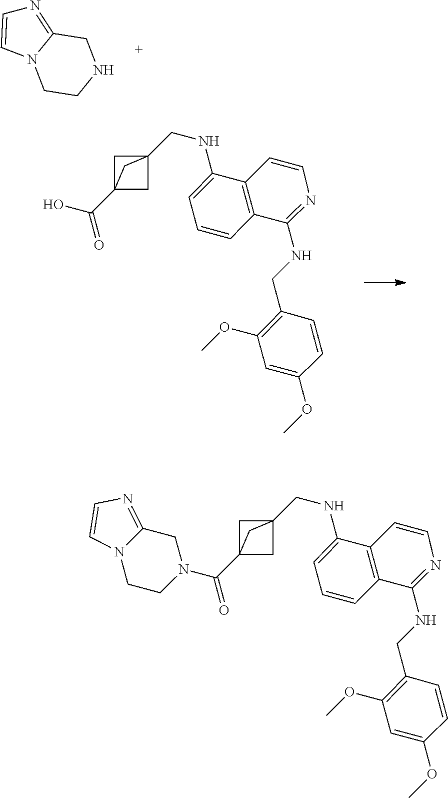 [SMILES: c1cn2c(n1)CNCC2 ; COc1ccc(CNc2nccc3c(NCC45CC(C(=O)O)(C4)C5)cccc23)c(OC)c1 ; COc1ccc(CNc2nccc3c(NCC45CC(C(=O)N6CCn7ccnc7C6)(C4)C5)cccc23)c(OC)c1]

5,6,7,8-Tetrahydroimidazo[1,2-a]pyrazine (23 mg, 0.18 mmol) and 3-[[[1-[(2,4-dimethoxyphenyl)methylamino]isoquinolin-5-yl]amino]methyl]bicyclo[1.1.1]pentane-1-carboxylic acid (80 mg, 0.18 mmol) were taken up in DCM (10 mL) and cooled in an ice/water bath. HOBt (34 mg, 0.22 mmol), TEA (129 μL, 0.92 mmol) and EDC (50 mg, 0.26 mmol) were added and the mixture stirred at rt for 6 days. The mixture was diluted with DCM (25 mL) and sat. NaHCO₃ (aq) 10 mL). The aqueous layer was re-extracted into DCM (25 mL) and the combined organic layers were dried (MgSO₄), filtered and concentrated. Flash chromatography (Silica, 0-20% MeOH in DCM) afforded the product (50 mg, 50% yield).

[M+H]⁺=539.3

### [3-[[(1-Aminoisoquinolin-5-yl)amino]methyl]-1-bicyclo[1.1.1]pentanyl]-(6,8-dihydro-5H-imidazo[1,2-a]pyrazin-7-yl)methanone (Example 1016)

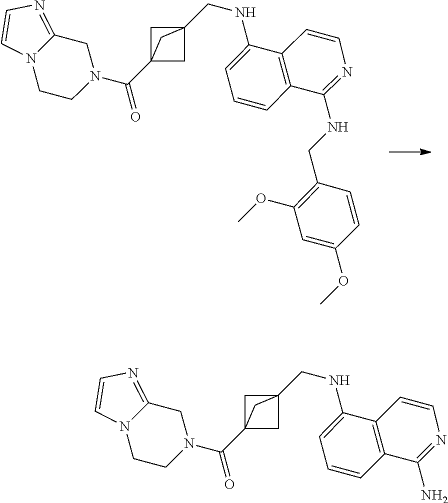 [SMILES: COc1ccc(CNc2nccc3c(NCC45CC(C(=O)N6CCn7ccnc7C6)(C4)C5)cccc23)c(OC)c1 ; Nc1nccc2c(NCC34CC(C(=O)N5CCn6ccnc6C5)(C3)C4)cccc12]

6,8-Dihydro-5H-imidazo[1,2-a]pyrazin-7-yl-[3-[[[1-[(2,4-dimethoxyphenyl)methylamino]isoquinolin-5-yl]amino]methyl]-1-bicyclo[1.1.1]pentanyl]methanone (50 mg, 0.09 mmol) was deprotected according to General Method 8 in DCM (1 mL). Following SCX the resulting residue was lyophilized to afford the product (35 mg, 97% yield).

[M+H]⁺=389.2

¹H NMR (DMSO-d6, 400 MHz) δ 2.08 (6H, s), 2.95-3.20 (1H, m), 3.81-4.05 (5H, m), 4.58-4.79 (2H, m), 5.86-5.93 (1H, m), 6.52 (2H, s), 6.65 (1H, d, J=7.7 Hz), 6.87 (1H, d, J=9.3 Hz), 7.09-7.23 (3H, m), 7.33 (1H, d, J=8.3 Hz), 7.72 (1H, d, J=6.2 Hz)

### Example Number 1017

### 5-N-[[3-(Imidazo[1,2-a]pyridin-6-yloxymethyl)-1-bicyclo[1.1.1]pentanyl]methyl]isoquinoline-1,5-diamine

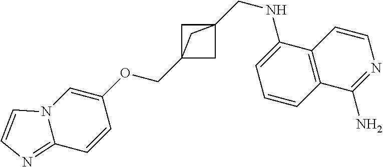 [SMILES: Nc1nccc2c(NCC34CC(COc5ccc6nccn6c5)(C3)C4)cccc12]

### 1-N-[(2,4-Dimethoxyphenyl)methyl]-5-N-[[3-(imidazo[1,2-a]pyridin-6-yloxymethyl)-1-bicyclo[1.1.1]pentanyl]methyl]isoquinoline-1,5-diamine

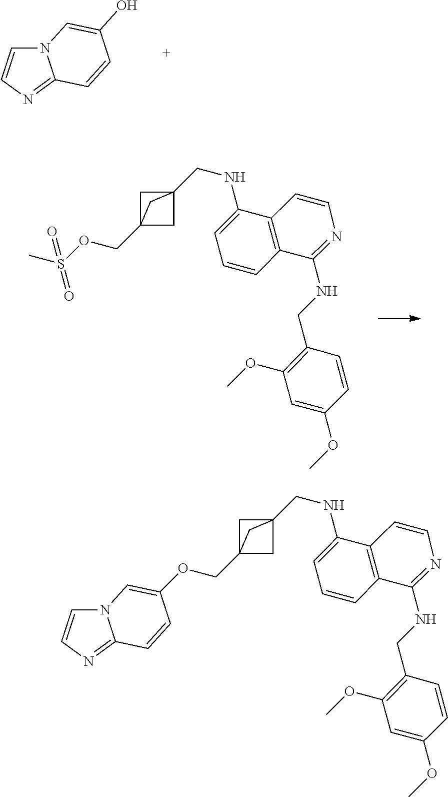

Following General Method 2b, [3-[[[1-[(2,4-dimethoxyphenyl)methylamino]isoquinolin-5-yl]amino]methyl]-1-bicyclo[1.1.1]pentanyl]methyl methanesulfonate (150 mg, 0.3 mmol) was reacted with imidazo[1,2-a]pyridin-6-ol (61 mg, 0.45 mmol) and K₂CO₃ (167 mg, 1.21 mmol) in MeCN (5 mL) at 90° C. for 3 days. Purification by flash chromatography (Silica, 0-20% (10% NH₃ in MeOH) in DCM) afforded the product (80 mg, 45% yield).

[M+H]⁺=536.3

### 5-N-[[3-(Imidazo[1,2-a]pyridin-6-yloxymethyl)-1-bicyclo[1.1.1]pentanyl]methyl]isoquinoline-1,5-diamine (Example 1017)

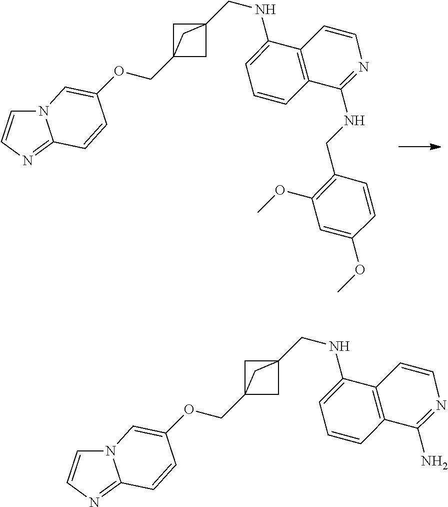 [SMILES: COc1ccc(CNc2nccc3c(NCC45CC(COc6ccc7nccn7c6)(C4)C5)cccc23)c(OC)c1 ; Nc1nccc2c(NCC34CC(COc5ccc6nccn6c5)(C3)C4)cccc12]

1-N-[(2,4-Dimethoxyphenyl)methyl]-5-N-[[3-(imidazo[1,2-a]pyridin-6-yloxymethyl)-1-bicyclo[1.1.1]pentanyl]methyl]isoquinoline-1,5-diamine (19 mg, 0.04 mmol) was deprotected according to General Method 8. After SCX, lyophilisation afforded the product (13 mg, 92% yield).

[M+H]⁺=386.2

¹H NMR (DMSO-d6, 400 MHz) δ 1.75 (6H, s), 3.96 (2H, s), 5.92 (1H, t, J=6.0 Hz), 6.62 (2H, s), 6.67 (1H, d, J=7.7 Hz), 6.99 (1H, dd, J=9.8, 2.4 Hz), 7.16 (1H, d, J=6.2 Hz), 7.21 (1H, t, J=8.0 Hz), 7.33 (1H, d, J=8.3 Hz), 7.44 (1H, d, J=9.7 Hz), 7.48 (1H, d, J=1.2 Hz), 7.70 (1H, d, J=6.2 Hz), 7.79 (1H, s), 8.23 (1H, d, J=2.4 Hz). 2H masked by water peak (confirmed by COSY)

### Example Number 1018

### 5-N-[[3-(Imidazo[1,2-a]pyridin-8-yloxymethyl)-1-bicyclo[1.1.1]pentanyl]methyl]isoquinoline-1,5-diamine

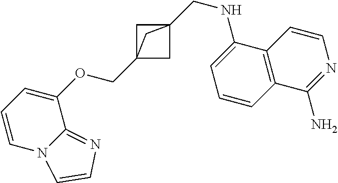 [SMILES: Nc1nccc2c(NCC34CC(COc5cccn6ccnc56)(C3)C4)cccc12]

### 1-N-[(2,4-Dimethoxyphenyl)methyl]-5-N-[[3-(imidazo[1,2-a]pyridin-8-yloxymethyl)-1-bicyclo[1.1.1]pentanyl]methyl]isoquinoline-1,5-diamine

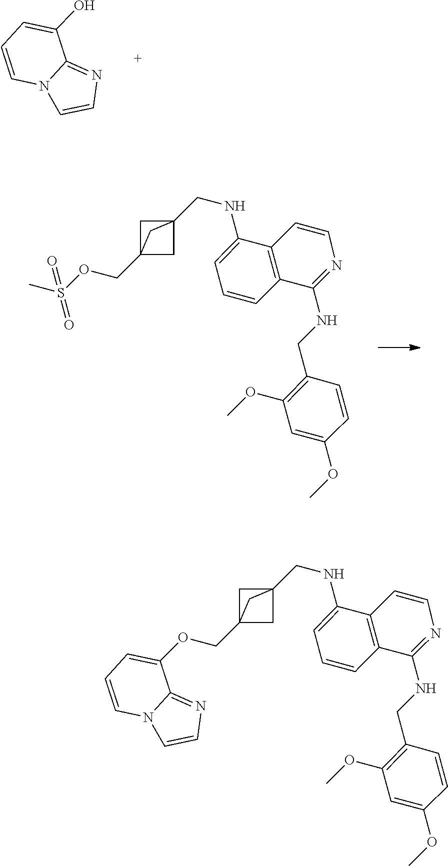 [SMILES: Oc1cccn2ccnc12 ; COc1ccc(CNc2nccc3c(NCC45CC(COS(C)(=O)=O)(C4)C5)cccc23)c(OC)c1 ; COc1ccc(CNc2nccc3c(NCC45CC(COc6cccn7ccnc67)(C4)C5)cccc23)c(OC)c1]

Following General Method 2b, [3-[[[1-[(2,4-dimethoxyphenyl)methylamino]isoquinolin-5-yl]amino]methyl]-1-bicyclo[1.1.1]pentanyl]methyl methanesulfonate (70 mg, 0.14 mmol) was reacted with imidazo[1,2-a]pyridin-8-ol (28 mg, 0.21 mmol) and K₂CO₃ (78 mg, 0.56 mmol) in MeCN (3 mL) under microwave heating at 120° C. for 90 min. After work up the crude material was purified by flash chromatography (Silica, 0-6% MeOH in DCM) to afford the product (37 mg, 49% yield).

[M+H]⁺=536.3

### 5-N-[[3-(Imidazo[1,2-a]pyridin-8-yloxymethyl)-1-bicyclo[1.1.1]pentanyl]methyl]isoquinoline-1,5-diamine (Example 1018)

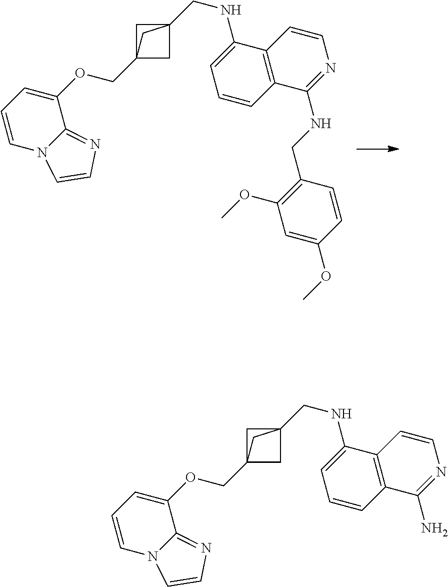 [SMILES: COc1ccc(CNc2nccc3c(NCC45CC(COc6cccn7ccnc67)(C4)C5)cccc23)c(OC)c1 ; Nc1nccc2c(NCC34CC(COc5cccn6ccnc56)(C3)C4)cccc12]

1-N-[(2,4-Dimethoxyphenyl)methyl]-5-N-[[3-(imidazo[1,2-a]pyridin-8-yloxymethyl)-1-bicyclo[1.1.1]pentanyl]methyl]isoquinoline-1,5-diamine (37 mg, 0.07 mmol) was deprotected according to General Method 8 at 50° C. for 60 min. Following SCX the crude product was purified by automated prep HPLC (mass directed 2-60% MeCN in water with a basic mobile phase of 0.1% NH₃ in water over 20 min) and lyophilised to afford the product (10 mg, 38% yield).

[M+H]⁺=386.2

¹H NMR (DMSO-d₆, 400 MHz) δ 1.76 (6H, s), 4.17 (2H, s), 5.86 (1H, t, J=5.9 Hz), 6.46 (2H, s), 6.58 (1H, d, J=7.5 Hz), 6.65 (1H, d, J=7.7 Hz), 6.72 (1H, t, J=7.1 Hz), 7.14 (1H, d, J=6.1 Hz), 7.19 (1H, t, J=8.0 Hz), 7.30 (1H, d, J=8.3 Hz), 7.44 (1H, s), 7.71 (1H, d, J=6.0 Hz), 7.89 (1H, s), 8.11 (1H, d, J=6.7 Hz). 2H masked by water peak (confirmed by COSY)

### Example Number 1019

### 4-[[3-[[(1-aminoisoquinolin-5-yl)amino]methyl]-1-bicyclo[1.1.1]pentanyl]methoxy]-1-methylpyridin-2-one

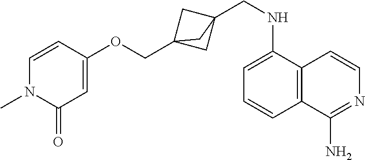 [SMILES: Cn1ccc(OCC23CC(CNc4cccc5c(N)nccc45)(C2)C3)cc1=O]

### 4-[[3-[[[1-[(2,4-dimethoxyphenyl)methylamino]isoquinolin-5-yl]amino]methyl]-1-bicyclo[1.1.1]pentanyl]methoxy]-1-methylpyridin-2-one

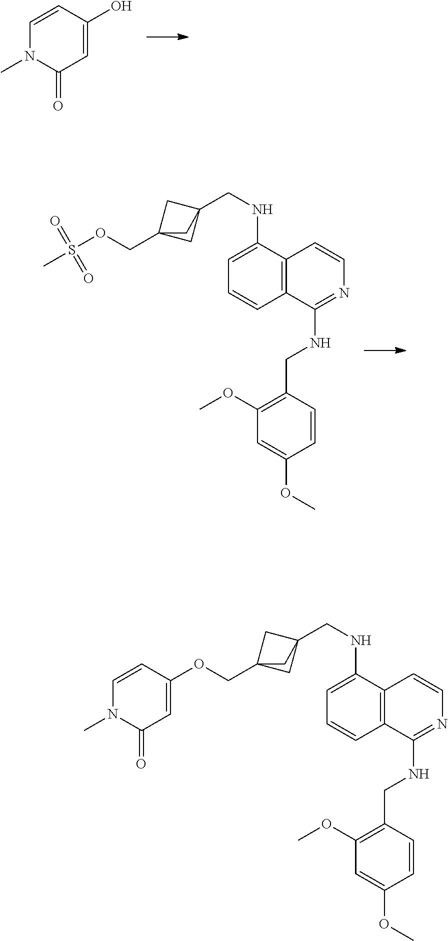

Following General Method 2b, [3-[[[1-[(2,4-dimethoxyphenyl)methylamino]isoquinolin-5-yl]amino]methyl]-1-bicyclo[1.1.1]pentanyl]methyl methanesulfonate (100 mg, 0.2 mmol) was reacted with 4-hydroxy-1-methylpyridin-2-one (38 mg, 0.3 mmol) and K₂CO₃ (111 mg, 0.8 mmol) in MeCN (5 mL) in a sealed vial at 90° C. for 3 days. Purification by flash chromatography (Silica, 0-20% MeOH in DCM) afforded the product (52 mg, 44% yield).

[M+H]⁺=527.3

### 4-[[3-[[(1-Aminoisoquinolin-5-yl)amino]methyl]-1-bicyclo[1.1.1]pentanyl]methoxy]-1-methylpyridin-2-one (Example 1019)

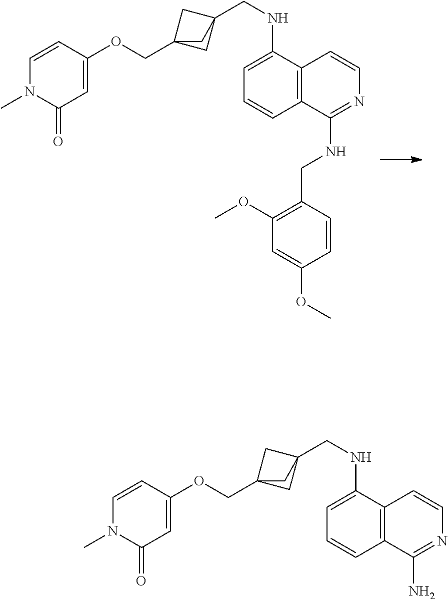 [SMILES: COc1ccc(CNc2nccc3c(NCC45CC(COc6ccn(C)c(=O)c6)(C4)C5)cccc23)c(OC)c1 ; Cn1ccc(OCC23CC(CNc4cccc5c(N)nccc45)(C2)C3)cc1=O]

Deprotection of 4-[[3-[[[1-[(2,4-dimethoxyphenyl)methylamino]isoquinolin-5-yl]amino]methyl]-1-bicyclo[1.1.1]pentanyl]methoxy]-1-methylpyridin-2-one (52 mg, 0.09 mmol) was carried out using General Method 8. After SCX and purification by automated prep HPLC (mass directed 2-60% MeCN in water with a basic mobile phase of 0.1% NH₃ in water over 20 min), lyophilisation afforded the product (6 mg, 18% yield).

[M+H]⁺=377.2

¹H NMR (DMSO-d6, 400 MHz) δ 1.71 (6H, s), 3.31 (3H, s), 3.95 (2H, s), 5.74 (1H, d, J=2.7 Hz), 5.86-5.90 (2H, m), 6.48 (2H, s), 6.64 (1H, d, J=7.7 Hz), 7.14 (1H, d, J=6.2 Hz), 7.20 (1H, t, J=8.0 Hz), 7.31 (1H, d, J=8.3 Hz), 7.53 (1H, d, J=7.7 Hz), 7.71 (1H, d, J=6.1 Hz). 2H masked by water peak.

### Example Number 1130

### 2-Chloro-N-[[3-(5,6,7,8-tetrahydroimidazo[1,2-a]pyridin-7-yloxymethyl)-1-bicyclo[1.1.1]pentanyl]methyl]-1H-pyrrolo[2,3-b]pyridin-4-amine

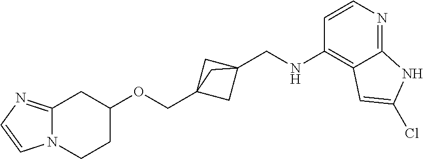 [SMILES: Clc1cc2c(NCC34CC(COC5CCn6ccnc6C5)(C3)C4)ccnc2[nH]1]

### 2-Chloro-N-[[3-(5,6,7,8-tetrahydroimidazo[1,2-a]pyridin-7-yloxymethyl)-1-bicyclo[1.1.1]pentanyl]methyl]-1H-pyrrolo[2,3-b]pyridin-4-amine (Example 1130)

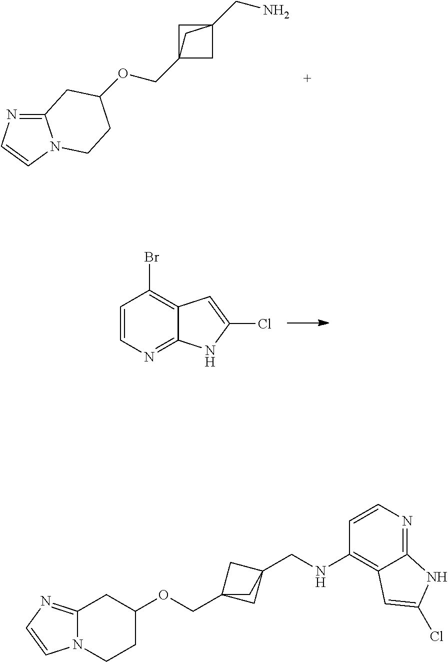 [SMILES: NCC12CC(COC3CCn4ccnc4C3)(C1)C2 ; Clc1cc2c(Br)ccnc2[nH]1 ; Clc1cc2c(NCC34CC(COC5CCn6ccnc6C5)(C3)C4)ccnc2[nH]1]

According to a modification of General Method 4, to a screw-capped pressure vial was added [3-(5,6,7,8-tetrahydroimidazo[1,2-a]pyridin-7-yloxymethyl)-1-bicyclo[1.1.1]pentanyl]methanamine (130 mg, 0.53 mmol) and 4-bromo-2-chloro-1H-pyrrolo[2,3-b]pyridine (122 mg, 0.53 mmol). THF (2 mL) was added followed by LiHMDS (1M in THF) (2.63 mL, 2.63 mmol). The solution was purged with nitrogen for 5 min before tert-BuBrettphos Pd G3 (22 mg, 0.03 mmol) was added. The vial was sealed and the mixture was purged with nitrogen for a further 5 minutes before stirring at 70° C. for 5 days. 1,4-Dioxane (5 mL), LiHMDS (1M in THF) (2.63 mL, 2.63 mmol) and Brettphos Pd G3 (24 mg, 0.03 mmol) were added and the reaction mixture was purged with nitrogen for 10 minutes. The reaction mixture was stirred at 70° C. overnight. The reaction was quenched with AcOH (2.0 eq) and concentrated. The crude was purified by an SCX eluting with NH₃ in MeOH followed by purification by flash chromatography and prep HPLC.

[M+H]⁺=398.2

¹H NMR (DMSO, 400 MHz) δ 1.59 (6H, s), 1.95-2.05 (2H, m), 2.68 (1H, dd, J=16.6, 5.6 Hz), 2.94 (1H, dd, J=16.5, 4.6 Hz), 3.30 (2H, d, J=6.0 Hz), 3.41-3.48 (2H, m), 3.90 (3H, td, J=6.2, 2.5 Hz), 6.14 (1H, d, J=5.8 Hz), 6.53 (1H, s), 6.57 (1H, s), 6.78 (1H, d, J=1.2 Hz), 6.96 (1H, d, J=1.2 Hz), 7.73 (1H, d, J=5.6 Hz), 11.93 (1H, s)

### Example Number 2020

### 5-N-[[1-(Imidazo[1,2-a]pyridin-7-yloxymethyl)-2-oxabicyclo[2.1.1]hexan-4-yl]methyl]isoquinoline-1,5-diamine

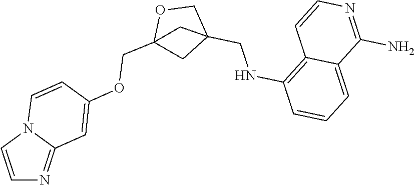 [SMILES: Nc1nccc2c(NCC34COC(COc5ccn6ccnc6c5)(C3)C4)cccc12]

### 1-N-[(2,4-Dimethoxyphenyl)methyl]-5-N-[[1-(imidazo[1,2-a]pyridin-7-yloxymethyl)-2-oxabicyclo[2.1.1]hexan-4-yl]methyl]isoquinoline-1,5-diamine

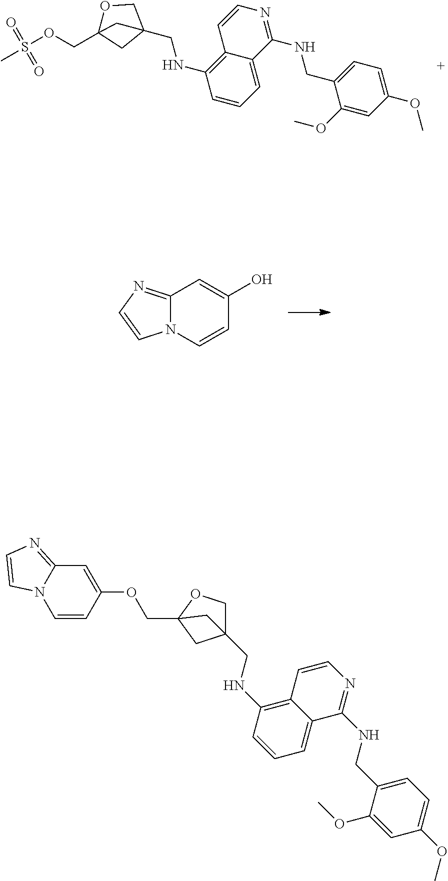

According to General Method 2a, to a solution of imidazo[1,2-a]pyridin-7-ol hydrochloride (100 mg, 0.59 mmol) and [4-[[[1-[(2,4-dimethoxyphenyl)methylamino]isoquinolin-5-yl]amino]methyl]-2-oxabicyclo[2.1.1]hexan-1-yl]methyl methanesulfonate (250 mg, 0.49 mmol) in DMF (6 mL) was added NaH (60% in mineral oil) (70 mg, 1.75 mmol) in an ice/water bath. The mixture was stirred for 5 min, then allowed to warm to rt for 5 min. The mixture was heated at 50° C. for 2 days, quenched with water (10 mL) and concentrated, azeotroping with toluene. Flash chromatography (silica, 0-15% MeOH in EtOAc) afforded the product (173 mg, 64% yield).

[M+H]⁺=552.3

### 5-N-[[1-(Imidazo[1,2-a]pyridin-7-yloxymethyl)-2-oxabicyclo[2.1.1]hexan-4-yl]methyl]isoquinoline-1,5-diamine (Example 2020)

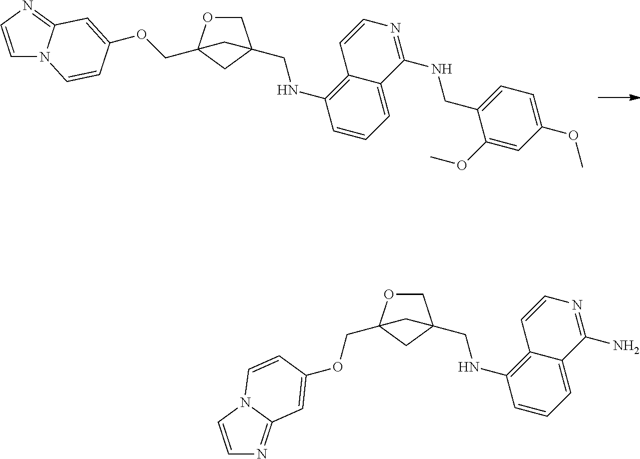 [SMILES: COc1ccc(CNc2nccc3c(NCC45COC(COc6ccn7ccnc7c6)(C4)C5)cccc23)c(OC)c1 ; Nc1nccc2c(NCC34COC(COc5ccn6ccnc6c5)(C3)C4)cccc12]

1-N-[(2,4-Dimethoxyphenyl)methyl]-5-N-[[1-(imidazo[1,2-a]pyridin-7-yloxymethyl)-2-oxabicyclo[2.1.1]hexan-4-yl]methyl]isoquinoline-1,5-diamine (137 mg, 0.25 mmol) was reacted according to General Method 8. The product was purified by flash chromatography (silica, 50-100% EtOAc in Pet. Ether followed by 0-17% MeOH in EtOAc) and lyophilized to afford the product (57 mg, 57% yield). LCMS: [M+H]⁺=402.2

¹H NMR (DMSO-d6, 400 MHz) δ 1.61 (2H, dd, J=4.4, 1.7 Hz), 1.85 (2H, dd, J=4.4, 1.7 Hz), 3.58 (2H, d, J=5.9 Hz), 3.71 (2H, s), 4.25 (2H, s), 5.99 (1H, t, J=5.9 Hz), 6.48 (2H, s), 6.59 (1H, dd, J=7.4, 2.5 Hz), 6.68 (1H, d, J=7.7 Hz), 6.93 (1H, d, J=2.5 Hz), 7.17 (1H, d, J=6.1 Hz), 7.21 (1H, t, J=8.0 Hz), 7.32 (1H, d, J=8.3 Hz), 7.37 (1H, d, J=1.3 Hz), 7.68-7.76 (2H, m), 8.36 (1H, d, J=7.4 Hz)

### Example Number 2022

### 5-N-[[1-(Imidazo[1,2-a]pyridin-6-yloxymethyl)-2-oxabicyclo[2.1.1]hexan-4-yl]methyl]isoquinoline-1,5-diamine

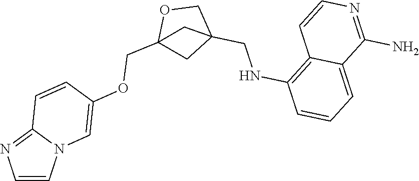 [SMILES: Nc1nccc2c(NCC34COC(COc5ccc6nccn6c5)(C3)C4)cccc12]

### 1-N-[(2,4-Dimethoxyphenyl)methyl]-5-N-[[1-(imidazo[1,2-a]pyridin-6-yloxymethyl)-2-oxabicyclo[2.1.1]hexan-4-yl]methyl]isoquinoline-1,5-diamine

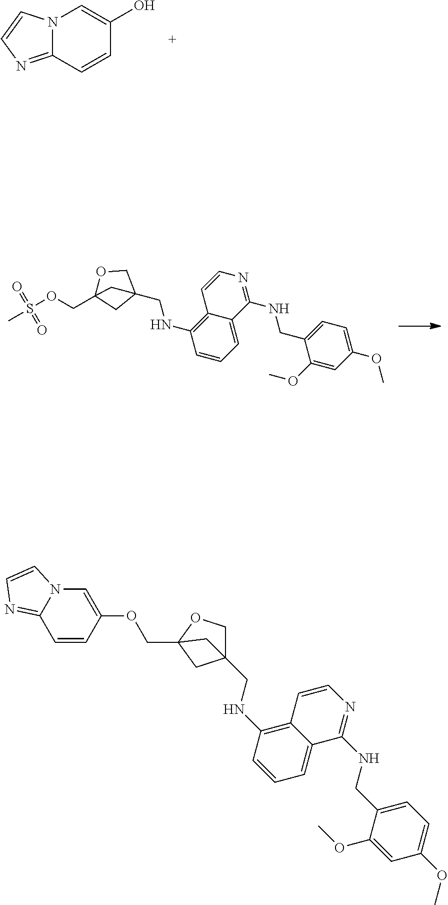

According to General Method 2b, [4-[[[1-[(2,4-dimethoxyphenyl)methylamino]isoquinolin-5-yl]amino]methyl]-2-oxabicyclo[2.1.1]hexan-1-yl]methyl methanesulfonate (265 mg, 0.52 mmol) was reacted with imidazo[1,2-a]pyridin-6-ol (104 mg, 0.77 mmol) and K₂CO₃ (285 mg, 2.06 mmol) in MeCN (7 mL) at 90° C. for 3 days. The mixture was cooled to rt and filtered through filter paper, washing with EtOAc (50 mL) and MeOH (10 mL), and the filtrate was concentrated. The residue was purified by flash chromatography (silica, 0-100% EtOAc in Pet. Ether followed by 0-20% MeOH in EtOAc) to afford the product (186 mg, 65% yield).

[M+H]⁺=552.3

### 5-N-[[1-(Imidazo[1,2-a]pyridin-6-yloxymethyl)-2-oxabicyclo[2.1.1]hexan-4-yl]methyl]isoquinoline-1,5-diamine (Example 2022)

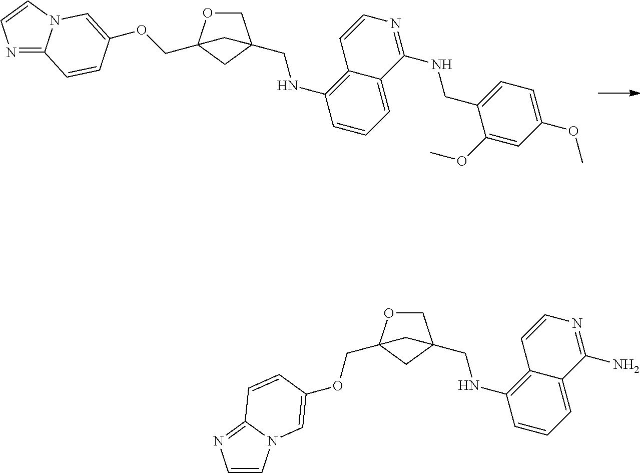 [SMILES: COc1ccc(CNc2nccc3c(NCC45COC(COc6ccc7nccn7c6)(C4)C5)cccc23)c(OC)c1 ; Nc1nccc2c(NCC34COC(COc5ccc6nccn6c5)(C3)C4)cccc12]

1-N-[(2,4-Dimethoxyphenyl)methyl]-5-N-[[1-(imidazo[1,2-a]pyridin-6-yloxymethyl)-2-oxabicyclo[2.1.1]hexan-4-yl]methyl]isoquinoline-1,5-diamine (186 mg, 0.34 mmol) was reacted according to General Method 8. The crude product was purified by flash chromatography (silica, 0-100% EtOAc in Pet. Ether followed by 0-17% MeOH in EtOAc) and lyophilized to afford the product (50 mg, 37% yield).

[M+H]⁺=402.2

¹H NMR (DMSO-d6, 400 MHz) δ 1.62 (2H, dd, J=4.5, 1.7 Hz), 1.85 (2H, dd, J=4.4, 1.7 Hz), 3.59 (2H, d, J=5.9 Hz), 3.71 (2H, s), 4.17 (2H, s), 6.00 (1H, t, J=6.0 Hz), 6.48 (2H, s), 6.69 (1H, d, J=7.7 Hz), 7.02 (1H, dd, J=9.7, 2.4 Hz), 7.13-7.27 (2H, m), 7.33 (1H, d, J=8.3 Hz), 7.42-7.51 (2H, m), 7.72 (1H, d, J=6.1 Hz), 7.81 (1H, t, J=0.9 Hz), 8.24-8.30 (1H, m)

### Example Number 6601

### 5-N-[[4-(Imidazo[1,2-a]pyridin-7-yloxymethyl)-2-oxabicyclo[2.1.1]hexan-1-yl]methyl]isoquinoline-1,5-diamine

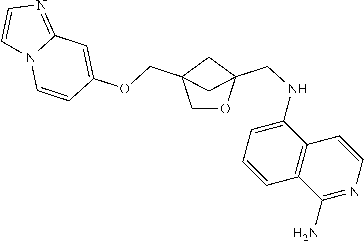 [SMILES: Nc1nccc2c(NCC34CC(COc5ccn6ccnc6c5)(CO3)C4)cccc12]

### 1-N-[(2,4-Dimethoxyphenyl)methyl]-5-N-[[4-(imidazo[1,2-a]pyridin-7-yloxymethyl)-2-oxabicyclo[2.1.1]hexan-1-yl]methyl]isoquinoline-1,5-diamine

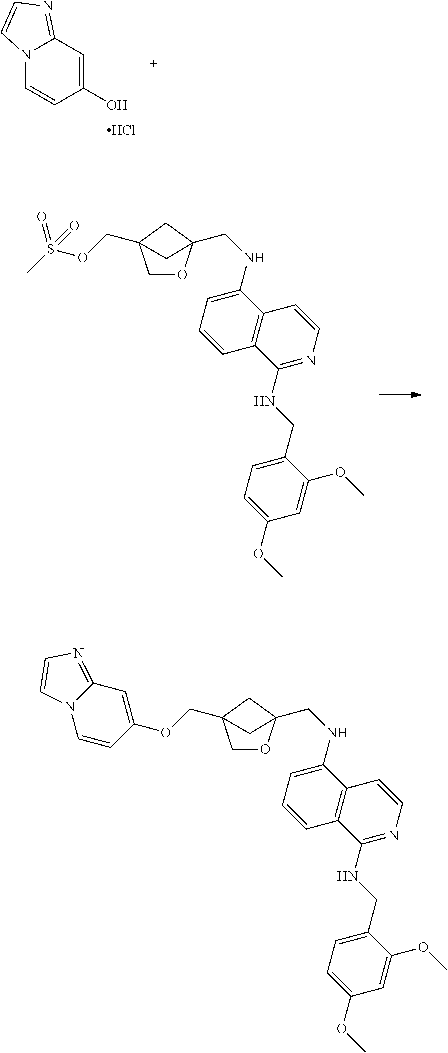

Following General Method 2a, imidazo[1,2-a]pyridin-7-ol hydrochloride (80 mg, 0.47 mmol) was reacted with [1-[[[1-[(2,4-dimethoxyphenyl)methylamino]isoquinolin-5-yl]amino]methyl]-2-oxabicyclo[2.1.1]hexan-4-yl]methyl methanesulfonate (200 mg, 0.39 mmol) in DMF (5 mL) with NaH (60% in mineral oil) (56 mg, 1.4 mmol) for 18 h. The reaction mixture was diluted with water (10 mL) and EtOAc (20 mL). The layers were separated, and the aqueous layers were back-extracted with EtOAc (3×10 mL). The organic layers were combined, dried (MgSO₄) and concentrated, azeotroping with toluene. Flash chromatography (silica, 0-15% MeOH in EtOAc) afforded the product (125 mg, 58% yield).

LCMS: [M+H]⁺=552.3

### 5-N-[[4-(Imidazo[1,2-a]pyridin-7-yloxymethyl)-2-oxabicyclo[2.1.1]hexan-1-yl]methyl]isoquinoline-1,5-diamine (Example 6601)

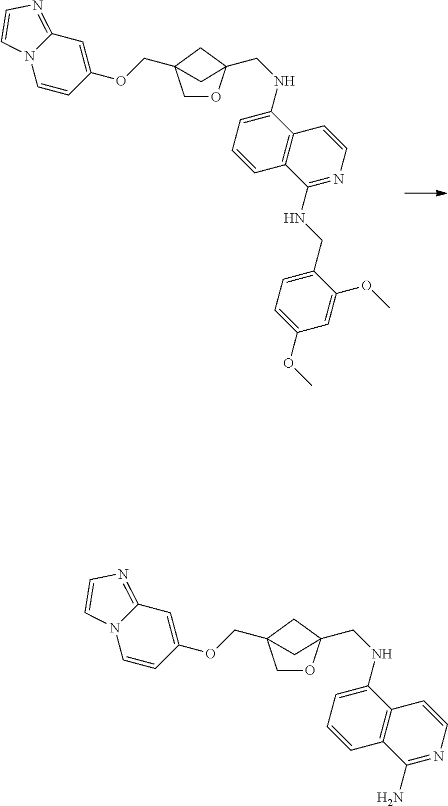 [SMILES: COc1ccc(CNc2nccc3c(NCC45CC(COc6ccn7ccnc7c6)(CO4)C5)cccc23)c(OC)c1 ; Nc1nccc2c(NCC34CC(COc5ccn6ccnc6c5)(CO3)C4)cccc12]

1-N-[(2,4-Dimethoxyphenyl)methyl]-5-N-[[4-(imidazo[1,2-a]pyridin-7-yloxymethyl)-2-oxabicyclo[2.1.1]hexan-1-yl]methyl]isoquinoline-1,5-diamine (125 mg, 0.23 mmol) was deprotected according to General Method 8, in TFA (2 mL, 26.12 mmol) at rt for 60 min. The reaction mixture was concentrated in vacuo before being passed directly through an SCX and washing with MeOH. The product was eluted with a solution of 7M NH₃ in MeOH and concentrated. The residue was purified by automated prep HPLC (mass directed 2-60% over 20 min in basic mobile phase) and lyophilised to afford to afford the product (52 mg, 57% yield) as a solid.

LCMS: [M+H]⁺=402.2

¹H NMR (CDCl₃, 400 MHz) δ 1.82-1.97 (4H, m), 3.63 (2H, d, J=5.2 Hz), 3.91 (2H, s), 4.28 (2H, s), 4.66 (1H, s), 5.10 (2H, s), 6.52 (1H, dd, J=7.4, 2.4 Hz), 6.75 (1H, d, J=7.7 Hz), 6.86 (1H, d, J=2.4 Hz), 7.00 (1H, d, J=6.1 Hz), 7.15 (1H, d, J=8.3 Hz), 7.36 (1H, t, J=8.0 Hz), 7.42 (1H, s), 7.49 (1H, d, J=1.4 Hz), 7.94 (2H, dd, J=6.8, 5.5 Hz) ppm.

### Example Number 6602

### 5-N-[[4-(Imidazo[1,2-a]pyridin-6-yloxymethyl)-2-oxabicyclo[2.1.1]hexan-1-yl]methyl]isoquinoline-1,5-diamine

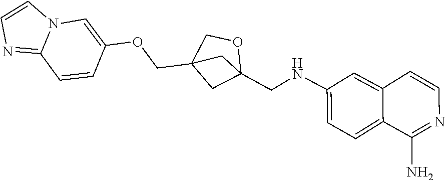 [SMILES: Nc1nccc2cc(NCC34CC(COc5ccc6nccn6c5)(CO3)C4)ccc12]

### tert-Butyl N-[[4-(hydroxymethyl)-2-oxabicyclo[2.1.1]hexan-1-yl]methyl]carbamate

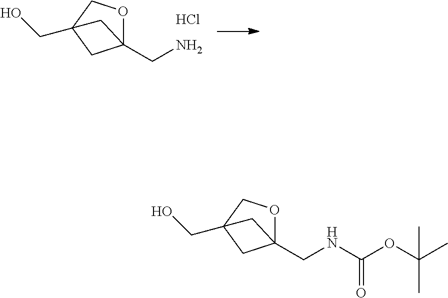 [SMILES: NCC12CC(CO)(CO1)C2 ; Cl ; CC(C)(C)OC(=O)NCC12CC(CO)(CO1)C2]

To a solution of [1-(aminomethyl)-2-oxabicyclo[2.1.1]hexan-4-yl]methanol hydrochloride (1000 mg, 5.57 mmol) and TEA (2716 μL, 19.5 mmol) in THF (30 mL) at 0° C. was added Boc₂O (1460 mg, 6.68 mmol) and the mixture was stirred for 10 min at 0° C. After that time the reaction was stirred for 18 h at rt. The reaction was quenched with water (30 mL) and washed with EtOAc (2×50 mL), dried (MgSO₄) and concentrated. The crude product was purified by flash chromatography (Silica, 0-20% MeOH in DCM) to afford the product (1010 mg, 75% yield).

[M+Na]⁺=266.0

¹H NMR (CDCl₃, 400 MHz) δ 1.45 (9H, s), 1.58 (2H, dd, J=4.6, 1.7 Hz), 1.68 (2H, dd, J=4.6, 1.7 Hz), 1.98-2.08 (1H, m), 3.46 (2H, d, J=6.0 Hz), 3.70 (2H, s), 3.89 (2H, d, J=4.7 Hz), 4.92 (1H, s)

### [1-[[2-Methylpropan-2-yl)oxycarbonylamino]methyl]-2-oxabicyclo[2.1.1]hexan-1-yl]methyl]methanesulfonate

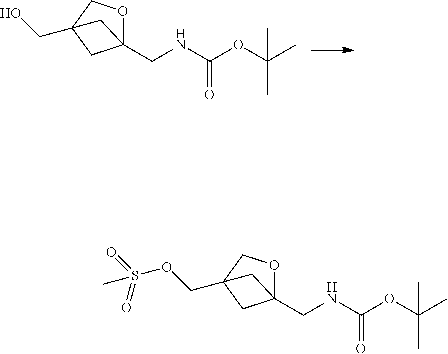

According to a modification of General method 1a, a solution of tert-butyl N-[[4-(hydroxymethyl)-2-oxabicyclo[2.1.1]hexan-1-yl]methyl]carbamate (1010 mg, 4.16 mmol) in DCM (30 mL) was cooled in an ice bath. MsCl (100 μL, 1.29 mmol) and TEA (547 mg, 5.4 mmol) were added dropwise sequentially whilst maintaining the temperature at 0° C. The mixture was stirred at rt for 90 min, after which time it was quenched with water (10 mL) and washed with DCM (2×25 mL). The combined organic layers were washed with water (3×25 mL), brine (20 mL), dried (MgSO₄) and concentrated. The productl crystalised on standing and was triturated with Et₂O (10 mL) to afford the product (1010 mg, 76% yield)

[M+Na]⁺=344.2

¹H NMR (CDCl₃, 400 MHz) δ 1.44 (9H, s), 1.67 (2H, dd, J=4.7, 1.7 Hz), 1.76 (2H, dd, J=4.7, 1.7 Hz), 3.04 (3H, s), 3.47 (2H, d, J=5.9 Hz), 3.73 (2H, s), 4.48 (2H, s), 4.84 (1H, s)

### N-[[4-(Imidazo[1,2-a]pyridin-6-yloxymethyl)-2-oxabicyclo[2.1.1]hexan-1-yl]methyl]carbamate

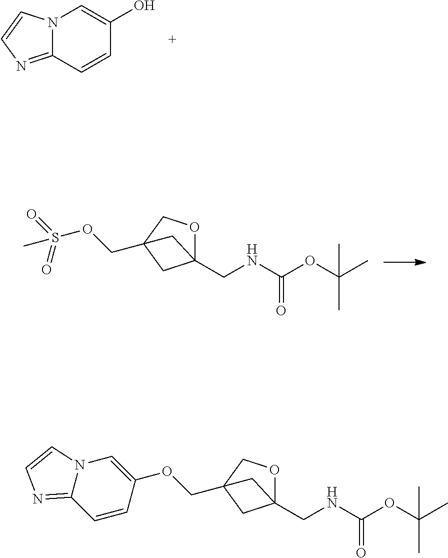 [SMILES: Oc1ccc2nccn2c1 ; CC(C)(C)OC(=O)NCC12CC(COS(C)(=O)=O)(CO1)C2 ; CC(C)(C)OC(=O)NCC12CC(COc3ccc4nccn4c3)(CO1)C2]

According to a modification of General Method 2a, [1-[[(2-methylpropan-2-yl)oxycarbonylamino]methyl]-2-oxabicyclo[2.1.1]hexan-4-yl]methyl methanesulfonate (100 mg, 0.31 mmol) and imidazo[1,2-a]pyridin-6-ol (83.0 mg, 0.62 mmol) were dissolved in DMF (3 mL). NaH, 60% w/w in mineral oil (37.0 mg, 0.93 mmol) and 4 Å molecular sieves were added and the reaction stirred at 50° C. for 18 h. The reaction was cooled to rt, quenched with brine (10 mL), filtered through Celite® and concentrated. The crude product was purified by flash chromatography (Silica, 0-25% MeOH in EtOAc) to afford the product (84.0 mg, 75% yield).

[M+H]⁺=360.2

### [4-(Imidazo[1,2-a]pyridin-6-yloxymethyl)-2-oxabicyclo[2.1.1]hexan-1-yl]methanamine

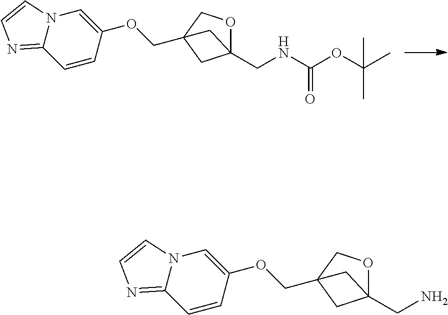 [SMILES: CC(C)(C)OC(=O)NCC12CC(COc3ccc4nccn4c3)(CO1)C2 ; NCC12CC(COc3ccc4nccn4c3)(CO1)C2]

According to a modification of General Method 5a, to a solution of tert-butyl N-[[4-(imidazo[1,2-a]pyridin-6-yloxymethyl)-2-oxabicyclo[2.1.1]hexan-1-yl]methyl]carbamate (84.0 mg, 0.23 mmol) in DCM (5 mL) was added 4M HCl in dioxane (950 μL, 3.8 mmol). The reaction was stirred at rt for 3 h and then concentrated and freebased using bicarbonate resin to afford the product (60.0 mg, 99% yield).

[M+H]⁺=260.2

### 1-N-[(2,4-Dimethoxyphenyl)methyl]-5-N-[[4-(imidazo[1,2-a]pyridin-6-yloxymethyl)-2-oxabicyclo[2.1.1]hexan-1-yl]methyl]isoquinoline-1,5-diamine

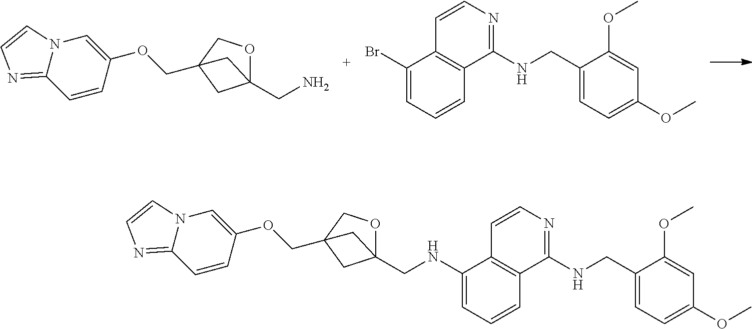 [SMILES: NCC12CC(COc3ccc4nccn4c3)(CO1)C2 ; COc1ccc(CNc2nccc3c(Br)cccc23)c(OC)c1 ; COc1ccc(CNc2nccc3c(NCC45CC(COc6ccc7nccn7c6)(CO4)C5)cccc23)c(OC)c1]

According to a modification of General Method 4, [4-(imidazo[1,2-a]pyridin-6-yloxymethyl)-2-oxabicyclo[2.1.1]hexan-1-yl]methanamine (60 mg, 0.23 mmol) was reacted with 5-bromo-N-(2,4-dimethoxybenzyl)isoquinolin-1-amine (130 mg, 0.35 mmol) in 1,4-dioxane (2 mL) in the presence of Brettphos Pd G4 (21 mg, 0.02 mmol) and NaOtBu (67.0 mg, 0.69 mmol) at 60° C. for 24 h. The crude residue was purified by flash chromatography (Silica, 0-20% MeOH in EtOAc) to afford the product (101 mg, 79% yield).

[M+H]⁺=552.3

### 5-N-[[4-(Imidazo[1,2-a]pyridin-6-yloxymethyl)-2-oxabicyclo[2.1.1]hexan-1-yl]methyl]isoquinoline-1,5-diamine (Example 6602)

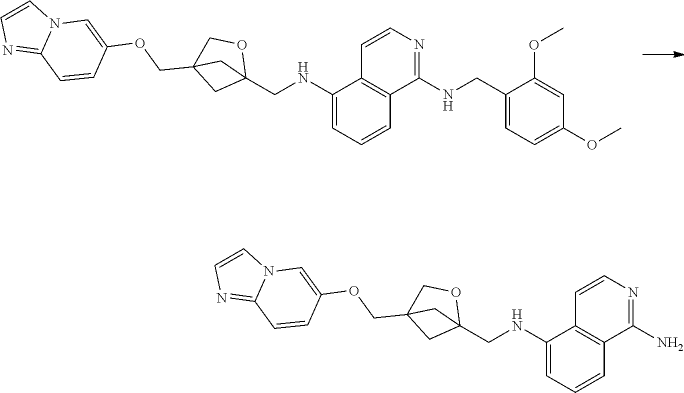 [SMILES: COc1ccc(CNc2nccc3c(NCC45CC(COc6ccc7nccn7c6)(CO4)C5)cccc23)c(OC)c1 ; Nc1nccc2c(NCC34CC(COc5ccc6nccn6c5)(CO3)C4)cccc12]

According to General method 8, 1-N-[(2,4-dimethoxyphenyl)methyl]-5-N-[[4-(imidazo[1,2-a]pyridin-6-yloxymethyl)-2-oxabicyclo[2.1.1]hexan-1-yl]methyl]isoquinoline-1,5-diamine (101 mg, 0.18 mmol) was deprotected. After SCX the crude was purified by automated prep HPLC (mass directed 2-60% over 20 min in a basic mobile phase) and lyophilised to afford the product (34.0 mg, 47% yield).

[M+H]⁺=402.2

¹H NMR (DMSO-d6, 400 MHz) δ 1.60 (2H, dd, J=4.5, 1.7 Hz), 1.86 (2H, dd, J=4.5, 1.7 Hz), 3.55 (2H, d, J=5.9 Hz), 3.74 (2H, s), 4.24 (2H, s), 5.92 (1H, t, J=6.0 Hz), 6.50 (2H, s), 6.70 (1H, d, J=7.7 Hz), 7.01 (1H, dd, J=9.7, 2.4 Hz), 7.11 (1H, d, J=6.1 Hz), 7.21 (1H, t, J=8.0 Hz), 7.34 (1H, d, J=8.3 Hz), 7.45 (1H, d, J=9.8 Hz), 7.49 (1H, d, J=1.1 Hz), 7.72 (1H, d, J=6.1 Hz), 7.80 (1H, t, J=0.9 Hz), 8.28 (1H, dd, J=2.5, 0.8 Hz)

### Example Number 6618

### 5-[[1-[[(1-Aminoisoquinolin-5-yl)amino]methyl]-2-oxabicyclo[2.1.1]hexan-4-yl]methoxy]-2,3-dihydroisoindol-1-one

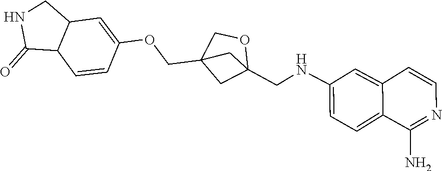 [SMILES: Nc1nccc2cc(NCC34CC(COC5=CC6CNC(=O)C6C=C5)(CO3)C4)ccc12]

### 5-[[1-[[[1-[(2,4-Dimethoxyphenyl)methylamino]isoquinolin-5-yl]amino]methyl]-2-oxabicyclo[2.1.1]hexan-4-yl]methoxy]-2,3-dihydroisoindol-1-one

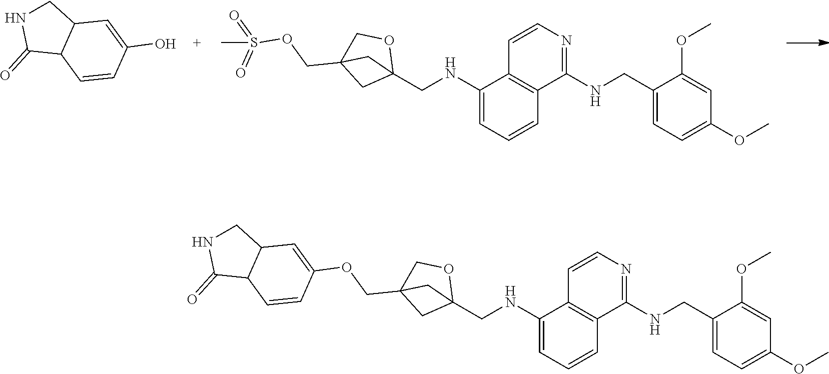

According to a modification of General Method 2a, [1-[[[1-[(2,4-dimethoxyphenyl)methylamino]isoquinolin-5-yl]amino]methyl]-2-oxabicyclo[2.1.1]hexan-4-yl]methyl methanesulfonate (400 mg, 0.78 mmol) was reacted with 5-hydroxyisoindolin-1-one (128 mg, 0.86 mmol) at 50° C. for 18 h. The reaction mixture was quenched with 1:1 brine/water (15 mL) and extracted with EtOAc (3×20 mL). Organic layers were combined, dried (MgSO₄) and concentrated. Residual DMF was removed by azeotroping with toluene. The crude material was purified via flash chromatography (Silica, 0-100% (2% NH₄OH in 3:3:1 EtOAc/MeCN/EtOH) in Pet. Ether) to afford the product (189 mg, 43% yield).

[M+H]⁺=567.5

### 5-[[1-[[(1-Aminoisoquinolin-5-yl)amino]methyl]-2-oxabicyclo[2.1.1]hexan-4-yl]methoxy]-2,3-dihydroisoindol-1-one (Example 6618)

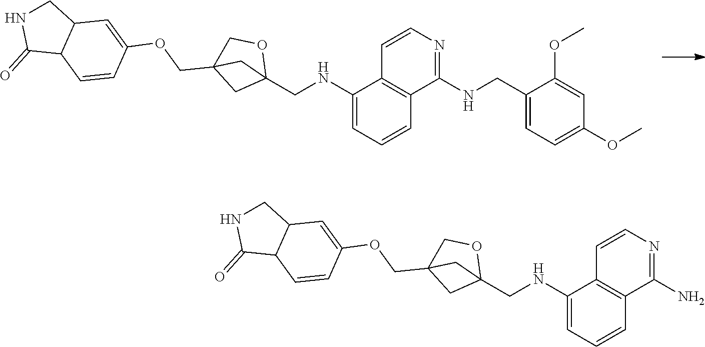 [SMILES: COc1ccc(CNc2nccc3c(NCC45CC(COC6=CC7CNC(=O)C7C=C6)(CO4)C5)cccc23)c(OC)c1 ; Nc1nccc2c(NCC34CC(COC5=CC6CNC(=O)C6C=C5)(CO3)C4)cccc12]

According to a modification of General method 8, 5-[[1-[[[1-[(2,4-dimethoxyphenyl)methylamino]isoquinolin-5-yl]amino]methyl]-2-oxabicyclo[2.1.1]hexan-4-yl]methoxy]-2,3-dihydroisoindol-1-one (189 mg, 0.33 mmol) was deprotected at rt for 2 h. After SCX, the crude was purified via prep-HPLC (mass directed 2-60% in basic mobile phase) then further purified via prep-HPLC (2-60% in acidic mobile phase). The material was lyophilised to afford the product (30 mg, 19% yield).

[M+H]⁺=417.4

¹H NMR (DMSO, 400 MHz) δ 1.58 (2H, dd, J=4.5, 1.7 Hz), 1.84 (2H, dd, J=4.6, 1.7 Hz), 3.54 (2H, d, J=5.6 Hz), 3.72 (2H, s), 4.28 (2H, s), 4.33 (2H, s), 5.94 (1H, t, J=6.0 Hz), 6.59 (2H, s), 6.70 (1H, d, J=7.7 Hz), 7.01 (1H, dd, J=8.4, 2.3 Hz), 7.08-7.16 (2H, m), 7.21 (1H, t, J=8.0 Hz), 7.34 (1H, d, J=8.3 Hz), 7.53 (1H, d, J=8.4 Hz), 7.71 (1H, d, J=6.1 Hz), 8.27 (1H, s), 8.31 (1H, s)

### Example Number 6624

### 5-N-[[4-[(3-Methylimidazo[1,2-a]pyridin-6-yl)oxymethyl]-2-oxabicyclo[2.1.1]hexan-1-yl]methyl]isoquinoline-1,5-diamine

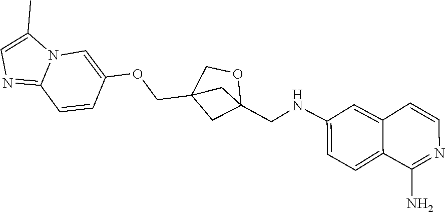 [SMILES: Cc1cnc2ccc(OCC34COC(CNc5ccc6c(N)nccc6c5)(C3)C4)cn12]

### 1-N-[(2,4-Dimethoxyphenyl)methyl]-5-N-[[4-[(3-methylimidazo[1,2-a]pyridin-6-yl)oxymethyl]-2-oxabicyclo[2.1.1]hexan-1-yl]methyl]isoquinoline-1,5-diamine

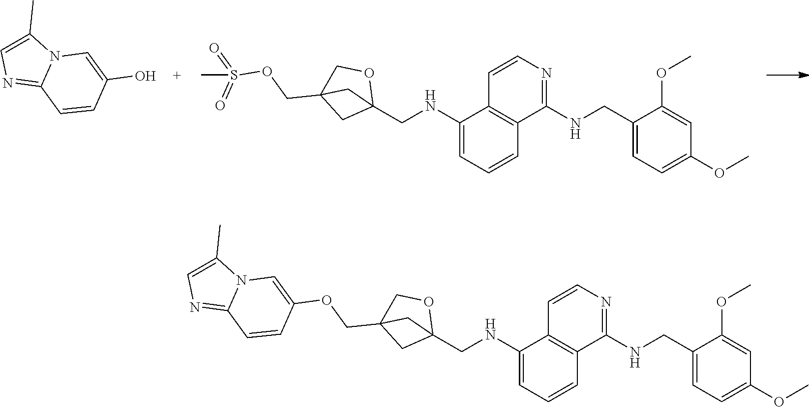

According to a modification of General Method 2a, 3-methylimidazo[1,2-a]pyridin-6-ol (75 mg, 0.38 mmol) was reacted with [1-[[[1-[(2,4-dimethoxyphenyl)methylamino]isoquinolin-5-yl]amino]methyl]-2-oxabicyclo[2.1.1]hexan-4-yl]methyl methanesulfonate (195 mg, 0.38 mmol) at 50° C. for 18 h. The reaction mixture was diluted with EtOAc (10 mL) and quenched with brine (10 mL). The layers were separated and the aq layer was back extracted with EtOAc (2×15 mL). The organic layers were combined, dried (MgSO₄) and concentrated. The residue was purified via flash chromatography (Silica, 0-100% (2% NH₄OH in 3:3:1 EtOAc/MeCN/EtOH+) in Pet. Ether) to afford the product (120 mg, 39% yield).

[M+H]⁺=566.6

### 5-N-[[4-[(3-Methylimidazo[1,2-a]pyridin-6-yl)oxymethyl]-2-oxabicyclo[2.1.1]hexan-1-yl]methyl]isoquinoline-1,5-diamine (Example 6624)

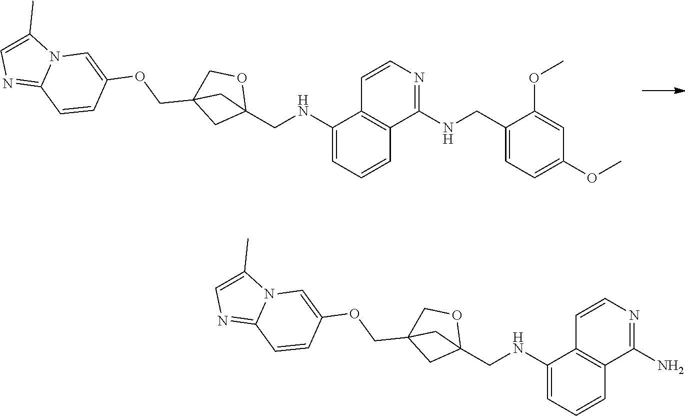 [SMILES: COc1ccc(CNc2nccc3c(NCC45CC(COc6ccc7ncc(C)n7c6)(CO4)C5)cccc23)c(OC)c1 ; Cc1cnc2ccc(OCC34COC(CNc5cccc6c(N)nccc56)(C3)C4)cn12]

According to a modification of General Method 8, a solution of 1-N-[(2,4-dimethoxyphenyl)methyl]-5-N-[[4-[(3-methylimidazo[1,2-a]pyridin-6-yl)oxymethyl]-2-oxabicyclo[2.1.1]hexan-1-yl]methyl]isoquinoline-1,5-diamine (120 mg, 0.15 mmol) in DCM (1 mL) was treated with TFA (2.10 mL, 27.26 mmol) and stirred at rt for 2 h and the reaction mixture was concentrated. After SCX work up the crude was purified via automated prep HPLC (mass directed 2-60% over 20 mins in acidic mobile phase) and lyophilized to afford the product (41 mg, 54% yield). (Example 6624).

[M+H]⁺=416.4

¹H (DMSO-d₆, 400 MHz) δ 1.61 (2H, dd, J=4.2, 1.2 Hz), 1.87 (2H, d, J=4.9 Hz), 2.41 (3H, s), 3.56 (2H, d, J=5.4 Hz), 3.75 (2H, s), 4.32 (2H, s), 5.95 (1H, t, J=5.8 Hz), 6.58 (2H, s), 6.71 (1H, d, J=7.3 Hz), 6.99 (1H, dd, J=9.7, 2.3 Hz), 7.11 (1H, d, J=6.2 Hz), 7.22 (1H, t, J=8.0 Hz), 7.29 (1H, s), 7.34 (1H, d, J=8.3 Hz), 7.42 (1H, d, J=9.7 Hz), 7.72 (1H, d, J=6.1 Hz), 7.83 (1H, d, J=2.1 Hz), 8.17 (2H, s)

### Example Number 6644

### 5-N-[[4-[[3-(Difluoromethyl)imidazo[1,2-a]pyridin-6-yl]oxymethyl]-2-oxabicyclo[2.1.1]hexan-1-yl]methyl]isoquinoline-1,5-diamine

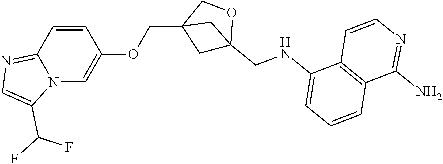 [SMILES: Nc1nccc2c(NCC34CC(COc5ccc6ncc(C(F)F)n6c5)(CO3)C4)cccc12]

### tert-ButylN-[[4-[[3-(difluoromethyl)imidazo[1,2-a]pyridin-6-yl]oxymethyl]-2-oxabicyclo[2.1.1]hexan-1-yl]methyl]carbamate

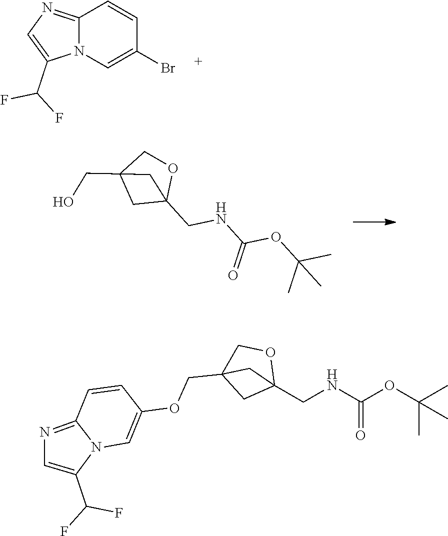 [SMILES: FC(F)c1cnc2ccc(Br)cn12 ; CC(C)(C)OC(=O)NCC12CC(CO)(CO1)C2 ; CC(C)(C)OC(=O)NCC12CC(COc3ccc4ncc(C(F)F)n4c3)(CO1)C2]

According to a modification of General Method 4, tert-Butyl N-[[4-(hydroxymethyl)-2-oxabicyclo[2.1.1]hexan-1-yl]methyl]carbamate (250 mg, 1.03 mmol) and 6-bromo-3-(difluoromethyl)imidazo[1,2-a]pyridine (254 mg, 1.03 mmol) were dissolved in 1,4-dioxane (1028 μL). The solution was degassed with N₂(g) for 5 min before CsCO₃ (502 mg, 1.54 mmol) and RockPhos Pd G3 (17 mg, 0.02 mmol) were added then degassed with N₂(g) for 5 min. The mixture was sonicated and vortexed until the solids had dissolved or were finely suspended. The mixture was heated at 100° C. for 60 min. The mixture was cooled, retreated with RockPhos Pd G3 (17 mg, 0.02 mmol), degassed with N₂(g) and stirred at 100° C. for 1 h. The reaction was cooled, retreated with RockPhos Pd G3 (17 mg, 0.02 mmol), degassed with nitrogen and stirred at 100° C. for 1 h. The reaction was cooled, retreated with RockPhos Pd G3 (17 mg, 0.02 mmol), degassed with nitrogen and stirred at 100° C. for 18 h. The reaction was cooled, diluted with EtOAc (10 mL), filtered through celite and concentrated. The product was purified by flash chromatography (Silica, 0-100% EtOAc in Pet. Ether followed by 0-20% MeOH in EtOAc) to afford the product, (239 mg, 57% yield) as a solid.

[M+H]⁺=410.2

### [4-[[3-(Difluoromethyl)imidazo[1,2-a]pyridin-6-yl]oxymethyl]-2-oxabicyclo[2.1.1]hexan-1-yl]methanamine

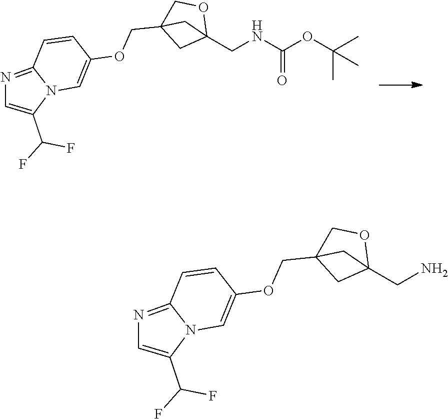 [SMILES: CC(C)(C)OC(=O)NCC12CC(COc3ccc4ncc(C(F)F)n4c3)(CO1)C2 ; NCC12CC(COc3ccc4ncc(C(F)F)n4c3)(CO1)C2]

According to a modification of General Method 5a, to a solution of tert-butyl N-[[4-[[3-(difluoromethyl) imidazo[1,2-a]pyridin-6-yl]oxymethyl]-2-oxabicyclo[2.1.1]hexan-1-yl]methyl]carbamate (230 mg, 0.56 mmol) in DCM (1 mL) was added 4M HCl in dioxane (702 μL, 2.81 mmol). The reaction was stirred at rt for 18 h and concentrated. The HCl salt was dissolved in methanol (5 ml), passed through bicarbonate resin cartridge twice and concentrated to afford the product, (155 mg, 89% yield) as an oil

[M+H]⁺=310.1

### 5-N-[[4-[[3-(Difluoromethyl)imidazo[1,2-a]pyridin-6-yl]oxymethyl]-2-oxabicyclo[2.1.1]hexan-1-yl]methyl]-1-N-[(2,4-dimethoxyphenyl)methyl]isoquinoline-1,5-diamine

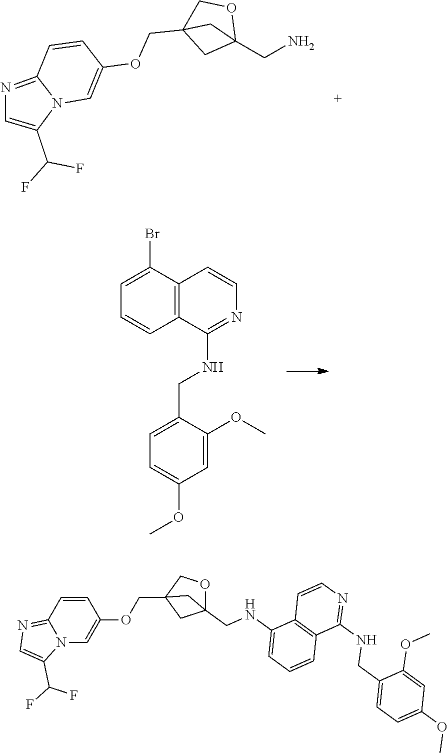 [SMILES: NCC12CC(COc3ccc4ncc(C(F)F)n4c3)(CO1)C2 ; COc1ccc(CNc2nccc3c(Br)cccc23)c(OC)c1 ; COc1ccc(CNc2nccc3c(NCC45CC(COc6ccc7ncc(C(F)F)n7c6)(CO4)C5)cccc23)c(OC)c1]

To a screw capped pressure vial equipped with a stirrer bar was added tris(dibenzylideneacetone)dipalladium(0) (23 mg, 0.03 mmol), [4-[[3-(difluoromethyl)imidazo[1,2-a]pyridin-6-yl]oxymethyl]-2-oxabicyclo[2.1.1]hexan-1-yl]methanamine (155 mg, 0.5 mmol), [1-(2-diphenylphosphanylnaphthalen-1-yl)naphthalen-2-yl]-diphenylphosphane (8 mg, 0.01 mmol), 5-bromo-N-[(2,4-dimethoxyphenyl)methyl]isoquinolin-1-amine (206 mg, 0.55 mmol) and NaOtBu (118 mg, 1.05 mmol). The vial was sealed and flushed with N₂(g) before 1,4-dioxane (5 mL) was added. The mixture was purged with N₂(g) for 5 min then stirred at 100° C. for 18 h. The mixture was cooled to rt, diluted with EtOAc (10 mL), filtered through a Celite, washed with EtOAc (3×10 mL) and concentrated. Flash chromatography (Silica, 0-100% EtOAc in Pet. Ether, followed by 0-20% MeOH in EtOAc) afforded the product, (193 mg, 64% yield).

[M+H]⁺=602.3

### 5-N-[[4-[[3-(Difluoromethyl)imidazo[1,2-a]pyridin-6-yl]oxymethyl]-2-oxabicyclo[2.1.1]hexan-1-yl]methyl]isoquinoline-1,5-diamine (Example 6644)

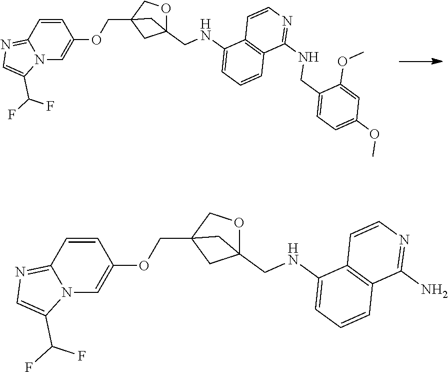 [SMILES: COc1ccc(CNc2nccc3c(NCC45CC(COc6ccc7ncc(C(F)F)n7c6)(CO4)C5)cccc23)c(OC)c1 ; Nc1nccc2c(NCC34CC(COc5ccc6ncc(C(F)F)n6c5)(CO3)C4)cccc12]

According to a modification of General Method 8, a solution of 5-N-[[4-[[3-(difluoromethyl)imidazo[1,2-a]pyridin-6-yl]oxymethyl]-2-oxabicyclo[2.1.1]hexan-1-yl]methyl]-1-N-[(2,4-dimethoxyphenyl)methyl]isoquinoline-1,5-diamine (193 mg, 0.32 mmol) in TFA (1220 μL, 16.0 mmol) was stirred at rt for 60 min. The solvent was removed and the resulting solid was dissolved in meOH (3 mL), filtered and passed through SCX-2 and washed with MeOH (10 mL). The product was eluted with a solution of 1M NH₃ in MeOH (10 mL) and concentrated. Flash chromatography (Silica, 0-100% EtOAc in MeCN, then 0-20% MeOH in EtOAc) afforded the product (119 mg, 82% yield).

[M+H]⁺=452.2

¹H NMR (DMSO-d6, 400 MHz) δ 1.61 (2H, dd, J=4.5, 1.7 Hz), 1.87 (2H, dd, J=4.5, 1.7 Hz), 3.56 (2H, d, J=5.9 Hz), 3.75 (2H, s), 4.32 (2H, s), 5.93 (1H, t, J=6.0 Hz), 6.50 (2H, s), 6.70 (1H, d, J=7.7 Hz), 7.11 (1H, d, J=6.1 Hz), 7.18-7.28 (2H, m), 7.34 (1H, d, J=8.3 Hz), 7.44 (1H, t, J=53.3 Hz), 7.61-7.64 (1H, m), 7.72 (1H, d, J=6.1 Hz), 7.85 (1H, t, J=2.0 Hz), 8.07 (1H, dd, J=2.4, 0.8 Hz).

### Example Numbers 8806 and 8807

### Trans* and cis* 5-(2-((imidazo[1,2-a]pyridin-7-yloxy)methyl)-6-azaspiro[3.4]octan-6-yl)isoquinolin-1-amine

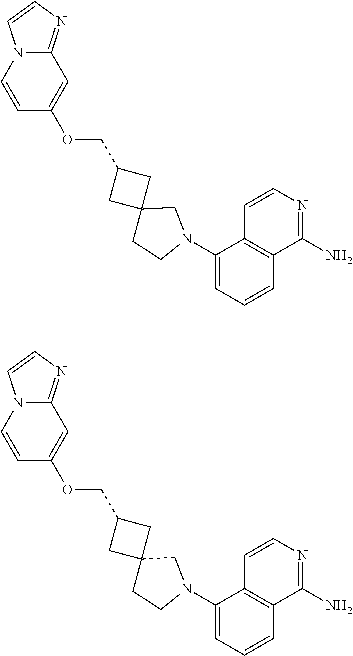

### tert-Butyl 2-(methylsulfonyloxymethyl)-6-azaspiro[3.4]octane-6-carboxylate

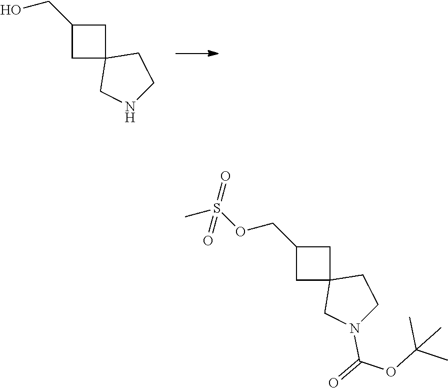 [SMILES: OCC1CC2(CCNC2)C1 ; CC(C)(C)OC(=O)N1CCC2(CC(COS(C)(=O)=O)C2)C1]

According to a modification of General Method 1a, to a solution of 6-azaspiro[3.4]octane-2-methanol (200 mg, 1.42 mmol) and TEA (987 μL, 7.08 mmol) in DCM (12 mL) at 0° C. was added Boc₂O (371 mg, 1.7 mmol) and the mixture was stirred for 1 h at 0° C. After that time MsCl (164 μL, 2.12 mmol) was added at 0° C. and the reaction stirred for 30 min. The reaction was quenched with water (50 mL), washed with DCM (2×70 mL), dried (MgSO₄) and concentrated. The material was triturated with Et₂O to afford the product (452 mg, 100% yield) as a solid.

[M+Na]⁺=342.1

### tert-Butyl 2-((imidazo[1,2-a]pyridin-7-yloxy)methyl)-6-azaspiro[3.4]octane-6-carboxylate

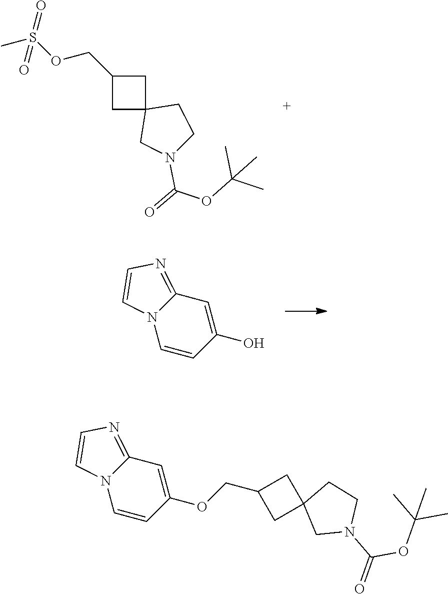 [SMILES: CC(C)(C)OC(=O)N1CCC2(CC(COS(C)(=O)=O)C2)C1 ; Oc1ccn2ccnc2c1 ; CC(C)(C)OC(=O)N1CCC2(CC(COc3ccn4ccnc4c3)C2)C1]

According to General Method 2b, tert-butyl 2-(((methylsulfonyl)oxy)methyl)-6-azaspiro[3.4]octane-6-carboxylate (250 mg, 783 μmol) was reacted with imidazo[1,2-a]pyridin-7-ol (157 mg, 1.17 mmol) and K₂CO₃ (324 mg, 2.35 mmol) in DMF (10 mL) at 60° C. for 18 h. The crude was purified by chromatography (C18, 0-95% MeCN/10 mM NH₄HCO₃) to afford the product (100 mg, 34%) as an oil.

[M+H]⁺=358.2

¹H NMR (CDCl3, 500 MHz) δ 1.44 (9H, d, J=3.4 Hz), 1.74-2.00 (4H, m), 2.00-2.19 (2H, m), 2.74 (1H, p, J=7.4 Hz), 3.20-3.40 (4H, m), 3.93 (2H, d, J=6.1 Hz), 6.49 (1H, dt, J=7.4, 2.6 Hz), 6.83 (1H, d, J=2.7 Hz), 7.39 (1H, s), 7.45 (1H, s), 7.92 (1H, d, J=7.3 Hz)

### 7-((6-Azaspiro[3.4]octan-2-yl)methoxy)imidazo[1,2-a]pyridine

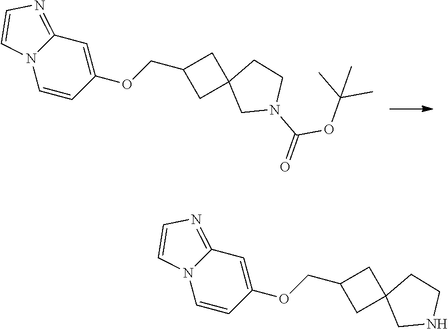 [SMILES: CC(C)(C)OC(=O)N1CCC2(CC(COc3ccn4ccnc4c3)C2)C1 ; c1cn2ccc(OCC3CC4(CCNC4)C3)cc2n1]

According to a modification of General Method 5b, tert-butyl 2-((imidazo[1,2-a]pyridin-7-yloxy)methyl)-6-azaspiro[3.4]octane-6-carboxylate (100 mg, 280 umol) was dissolved in DCM (9 mL) followed by the addition of TFA (1 g, 0.01 mol). The reaction mixture was stirred for 1.5 h before being concentrated. SCX yielded the product (50 mg, 66%) as an oil.

[M+H]⁺=258.2

### N-(2,4-Dimethoxybenzyl)-5-(2-((imidazo[1,2-a]yridine-7-yloxy)methyl)-6-azaspiro[3.4]octan-6-yl)isoquinolin-1-amine

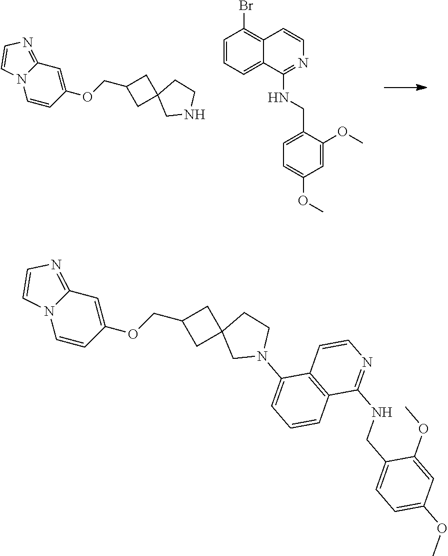 [SMILES: c1cn2ccc(OCC3CC4(CCNC4)C3)cc2n1 ; COc1ccc(CNc2nccc3c(Br)cccc23)c(OC)c1 ; COc1ccc(CNc2nccc3c(N4CCC5(CC(COc6ccn7ccnc7c6)C5)C4)cccc23)c(OC)c1]

According to a modification of General Method 4, 7-((6-azaspiro[3.4]octan-2-yl)methoxy)imidazo[1,2-a]pyridine (50 mg, 0.19 mmol) was reacted with 5-bromo-N-(2,4-dimethoxybenzyl)isoquinolin-1-amine (73 mg, 0.19 mmol), RuPhos Pd G3 (8.1 mg, 9.7 μmol) and NaO^{t}Bu (37 mg, 0.39 mmol) in 1,4-dioxane (2 mL) at 80° C. for 18 h. Work up and SCX afforded the product (85 mg, 52% yield) as an oil.

[M+H]⁺=550.2

### 5-(2-(Imidazo[1,2-a]pyridin-7-yloxy)methyl)-6-azaspiro[3.4]octan-6-ylisoquinolin-1-amine (Examples 8806 and 8807)

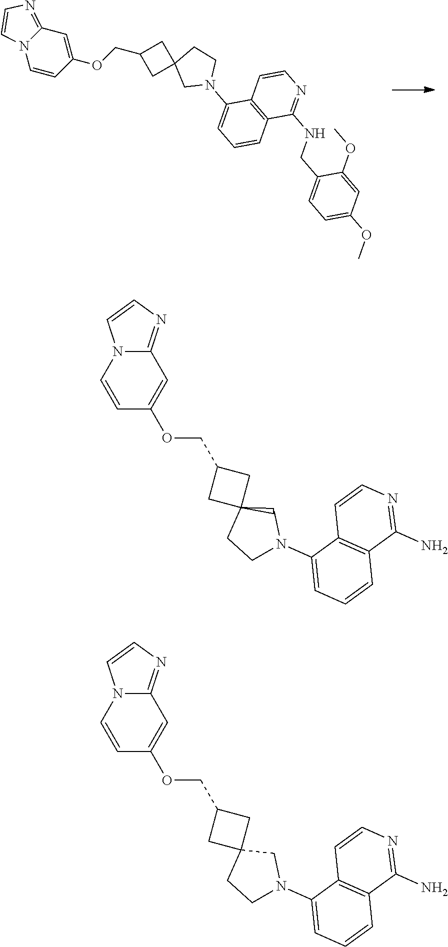

According to a modification of General Method 8, N-(2,4-dimethoxybenzyl)-5-(2-((imidazo[1,2-a]pyridin-7-yloxy)methyl)-6-azaspiro[3.4]octan-6-yl)isoquinolin-1-amine (85 mg, 0.12 mmol) was dissolved in DCM (9 mL) followed by the addition of TFA (1 mL). The reaction mixture was warmed to rt and stirred for 1.5 h before being concentrated. After SCX, purification by automated prep. HPLC (mass directed 20-100% over 12.5 min in basic mobile phase) afforded the trans* and cis* isomers.

### Example 8806

¹H NMR (CD₃OD, 500 MHz) δ 2.09 (4H, td, J=7.4, 3.9 Hz), 2.29-2.37 (2H, m), 2.82 (1H, tt, J=8.5, 6.3 Hz), 3.42 (2H, t, J=6.8 Hz), 3.51 (2H, s), 4.09 (2H, d, J=6.4 Hz), 6.66 (1H, dd, J=7.4, 2.4 Hz), 6.88 (1H, d, J=2.5 Hz), 7.15 (1H, dd, J=7.8, 0.9 Hz), 7.31 (1H, dd, J=6.3, 1.0 Hz), 7.36-7.44 (2H, m), 7.62 (1H, dt, J=8.4, 1.0 Hz), 7.66 (1H, dd, J=1.5, 0.7 Hz), 7.71 (1H, d, J=6.3 Hz), 8.28 (1H, dd, J=7.4, 0.7 Hz) [M+H]⁺=400.2

### Example 8807

¹H NMR (CD₃OD, 500 MHz) δ 2.05-2.14 (2H, m), 2.18 (2H, t, J=6.6 Hz), 2.22-2.30 (2H, m), 2.87 (1H, tt, J=8.3, 6.1 Hz), 3.43 (2H, s), 3.48 (2H, q, J=7.2 Hz), 4.05 (2H, d, J=6.2 Hz), 6.59-6.69 (1H, m), 6.85 (1H, d, J=2.5 Hz), 7.11 (1H, dd, J=7.8, 0.9 Hz), 7.29 (1H, dd, J=6.4, 1.0 Hz), 7.34-7.49 (2H, m), 7.59 (1H, dt, J=8.4, 1.0 Hz), 7.61-7.73 (2H, m), 8.22-8.31 (1H, m)

[M+H]⁺=400.2

### Example Number 10901

### N5-((5-((Imidazo[1,2-a]pyridin-7-yloxy)methyl)-2-oxabicyclo[3.1.1]heptan-1-yl)methyl)isoquinoline-1,5-diamine

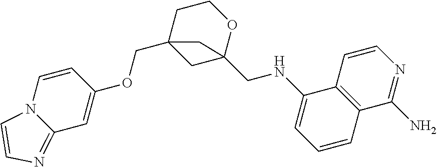 [SMILES: Nc1nccc2c(NCC34CC(COc5ccn6ccnc6c5)(CCO3)C4)cccc12]

### 1-N-[(2,4-Dimethoxyphenyl)methyl]-5-N-[[5-(imidazo[1,2-a]pyridin-7-yloxymethyl)-2-oxabicyclo[3.1.1]heptan-1-yl]methyl]isoquinoline-1,5-diamine

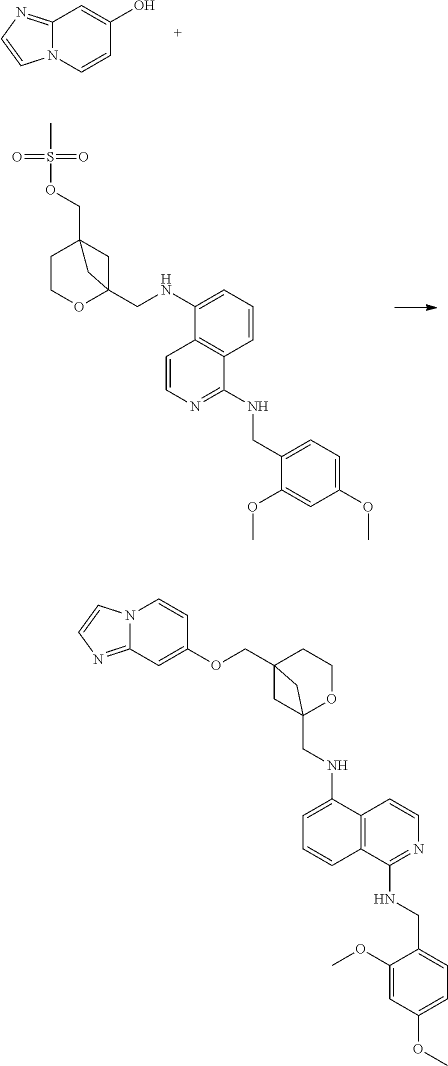 [SMILES: Oc1ccn2ccnc2c1 ; COc1ccc(CNc2nccc3c(NCC45CC(COS(C)(=O)=O)(CCO4)C5)cccc23)c(OC)c1 ; COc1ccc(CNc2nccc3c(NCC45CC(COc6ccn7ccnc7c6)(CCO4)C5)cccc23)c(OC)c1]

Following General Method 2a, imidazo[1,2-a]pyridin-7-ol hydrochloride (55 mg, 0.32 mmol) was reacted with[1-[[[1-[(2,4-dimethoxyphenyl)methylamino]isoquinolin-5-yl]amino]methyl]-2-oxabicyclo[3.1.1]heptan-5-yl]methyl methanesulfonate (190 mg, 0.32 mmol) in DMF (4 mL) with NaH (32 mg, 0.81 mmol). The mixture was filtered through filter paper, washed with EtOAc (50 ml) and MeOH (10 mL) and the filtrate was concentrated by azeotroping with toluene. The residue was taken up in DCM and concentrated onto silica before being purified by flash chromatography (Silica, 0-100% EtOAc in Pet. Ether followed by 0-20% MeOH in EtOAc) to afford the product (91 mg, 50% yield).

[M+H]⁺=566.2

### N5-((5-((Imidazo[1,2-a]pyridin-7-yloxy)methyl)-2-oxabicyclo[3.1.1]heptan-1-yl)methyl)isoquinoline-1,5-diamine (Example 10901)

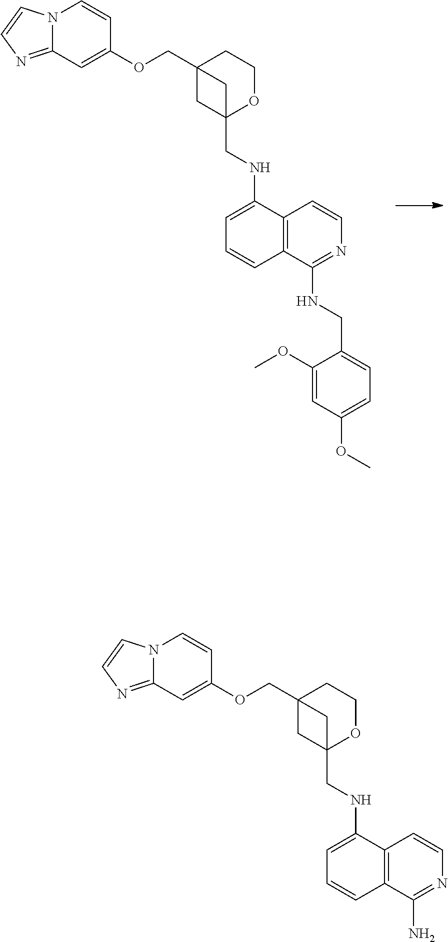 [SMILES: COc1ccc(CNc2nccc3c(NCC45CC(COc6ccn7ccnc7c6)(CCO4)C5)cccc23)c(OC)c1 ; Nc1nccc2c(NCC34CC(COc5ccn6ccnc6c5)(CCO3)C4)cccc12]

1-N-[(2,4-Dimethoxyphenyl)methyl]-5-N-[[5-(imidazo[1,2-a]pyridin-7-yloxymethyl)-2-oxabicyclo[3.1.1]heptan-1-yl]methyl]isoquinoline-1,5-diamine (91 mg, 0.16 mmol) was deprotected according to General Method 8, in TFA (2601 μL, 33.76 mmol) at rt for 60 min. After SCX, lyophilisation afforded the product (52 mg, 78% yield) as a solid. [M+H]⁺=416.2

¹H NMR (CDCl₃, 400 MHz) δ 2.03 (2H, dd, J=8.4, 6.0 Hz), 2.09 (2H, td, J=6.6, 5.8, 2.6 Hz), 2.15 (2H, t, J=6.9 Hz), 3.32 (2H, d, J=4.4 Hz), 3.86 (2H, s), 4.26 (2H, t, J=6.8 Hz), 4.65 (1H, s), 5.13 (2H, s), 6.51 (1H, dd, J=7.4, 2.5 Hz), 6.73 (1H, d, J=7.7 Hz), 6.83 (1H, d, J=2.4 Hz), 7.01 (1H, dd, J=6.1, 1.0 Hz), 7.13 (1H, d, J=8.3 Hz), 7.36 (1H, t, J=8.0 Hz), 7.41 (1H, d, J=1.2 Hz), 7.48 (1H, d, J=1.4 Hz), 7.93 (2H, dd, J=6.8, 3.8 Hz) ppm.

### Example Number 10907

### N-[[5-(Imidazo[1,2-a]pyridin-7-yloxymethyl)-2-oxabicyclo[3.1.1]heptan-1-yl]methyl]-2-methyl-1H-pyrrolo[2,3-b]pyridin-4-amine

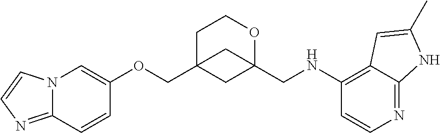 [SMILES: Cc1cc2c(NCC34CC(COc5ccc6nccn6c5)(CCO3)C4)ccnc2[nH]1]

### N-[[5-(Imidazo[1,2-a]pyridin-7-yloxymethyl)-2-oxabicyclo[3.1.1]heptan-1-yl]methyl]-2-methyl-1H-pyrrolo[2,3-b]pyridin-4-amine (Example 10907)

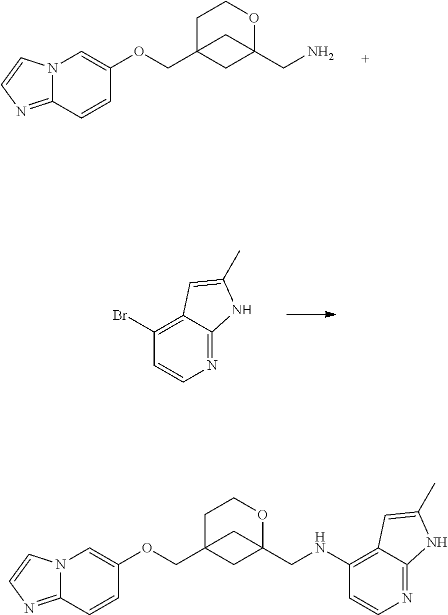 [SMILES: NCC12CC(COc3ccc4nccn4c3)(CCO1)C2 ; Cc1cc2c(Br)ccnc2[nH]1 ; Cc1cc2c(NCC34CC(COc5ccc6nccn6c5)(CCO3)C4)ccnc2[nH]1]

According to a modification of General Method 4, [[5-(imidazo[1,2-a]pyridin-7-yloxymethyl)-2-oxabicyclo[3.1.1]heptan-1-yl]methanamine (135 mg, 0.37 mmol) was reacted with 4-bromo-2-methyl-1H-pyrrolo[2,3-b]pyridine (77.0 mg, 0.37 mmol) in THF (5 mL) in the presence of LiHMDS (1M in THF) (183 μl, 1.83 mmol) and Brettphos Pd G3 (17 mg, 0.02 mmol) at 60° C. in a sealed vial for 6 days. The product was purified by flash chromatography (Silica, 0-100% EtOAc in Pet. ether followed by 0-15% MeOH in EtOAc) followed by automated prep HPLC (mass directed 2-60% over 20 min in basic mobile phase) and lyophilised to afford the product (22.0 mg, 15% yield).

[M+H]⁺=404.2

¹H NMR (DMSO-d₆, 400 MHz) δ 1.80 (2H, dd, J=7.0, 2.5 Hz), 1.95 (2H, d, J=7.8 Hz), 2.02 (2H, t, J=6.8 Hz), 2.30 (3H, d, J=1.0 Hz), 3.25 (2H, d, J=6.2 Hz), 3.86 (2H, s), 4.07 (2H, t, J=6.8 Hz), 6.09 (1H, d, J=5.6 Hz), 6.14 (1H, t, J=6.2 Hz), 6.25 (1H, dd, J=2.1, 1.1 Hz), 6.51 (1H, dd, J=7.4, 2.5 Hz), 6.87 (1H, d, J=2.5 Hz), 7.35 (1H, d, J=1.3 Hz), 7.68 (1H, d, J=5.5 Hz), 7.71 (1H, dd, J=1.3, 0.7 Hz), 8.34 (1H, dd, J=7.4, 0.7 Hz), 10.91 (1H, s)

### Example Number 10909

### 2-Chloro-N-[[5-(imidazo[1,2-a]pyridin-6-yloxymethyl)-2-oxabicyclo[3.1.1]heptan-1-yl]methyl]-1H-pyrrolo[2,3-b]pyridin-4-amine

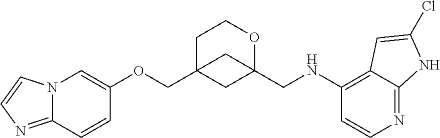 [SMILES: Clc1cc2c(NCC34CC(COc5ccc6nccn6c5)(CCO3)C4)ccnc2[nH]1]

### 2-Chloro-N-[[5-(imidazo[1,2-a]pyridin-6-yloxymethyl)-2-oxabicyclo[3.1.1]heptan-1-yl]methyl]-1H-pyrrolo[2,3-b]pyridin-4-amine (Example 10909)

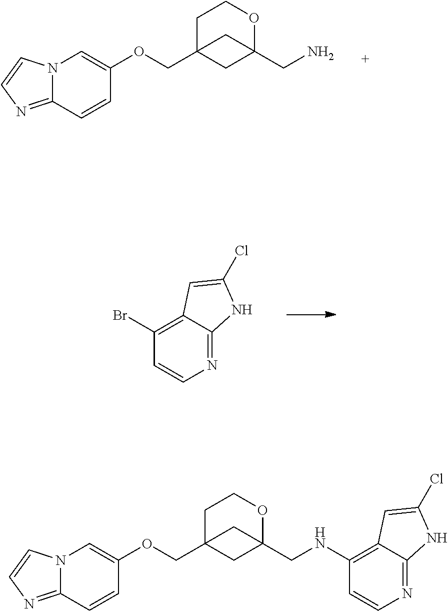 [SMILES: NCC12CC(COc3ccc4nccn4c3)(CCO1)C2 ; Clc1cc2c(Br)ccnc2[nH]1 ; Clc1cc2c(NCC34CC(COc5ccc6nccn6c5)(CCO3)C4)ccnc2[nH]1]

According to a modification of General Method 4, [5-(imidazo[1,2-a]pyridin-6-yloxymethyl)-2-oxabicyclo[3.1.1]heptan-1-yl]methanamine (26.0 mg, 0.1 mmol) was reacted with 4-bromo-2-chloro-1H-pyrrolo[2,3-b]pyridine (20.0 mg, 0.09 mmol) in THF (5 mL) in the presence of Brettphos Pd G3 (4 mg) and LiHMDS (1M in THF) (207 μL, 0.21 mmol) at 70° C. for 5 h. After work up and SCX the residue was purified by flash chromatography (Silica, 0-24% (10% NH₃ in MeOH) in DCM) and lyophilised to afford the product (4 mg, 8% yield).

[M+H]⁺=424.1

¹H NMR (CD₃CN) δ 1.82 (2H, dd, J=7.2, 2.4 Hz), 1.89-1.92 (2H, m), 2.00 (2H, t, J=6.8 Hz), 3.26 (2H, d, J=6.0 Hz), 3.71 (2H, s), 4.05 (2H, t, J=6.8 Hz), 4.99 (1H, s), 5.16 (1H, t, J=5.8 Hz), 6.19 (1H, t, J=6.0 Hz), 6.35 (1H, s), 6.86 (1H, dd, J=9.8, 2.3 Hz), 7.31 (1H, d, J=9.7 Hz), 7.38 (1H, d, J=0.8 Hz), 7.55 (1H, s), 7.77 (1H, J=5.7 Hz), 7.82 (1H, d, J=2.0 Hz)

### Example Number 10926

### 5-N-[[5-([1,2,4]Triazolo[4,3-a]pyridin-7-yloxymethyl)-2-oxabicyclo[3.1.1]heptan-1-yl]methyl]isoquinoline-1,5-diamine

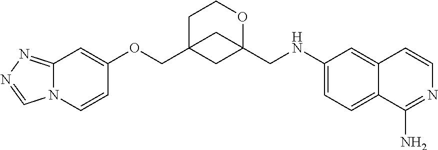 [SMILES: Nc1nccc2cc(NCC34CC(COc5ccn6cnnc6c5)(CCO3)C4)ccc12]

### [1.2.4]Triazolo[4,3-a]pyridin-7-ol

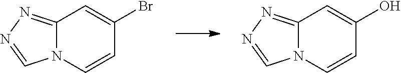 [SMILES: Brc1ccn2cnnc2c1 ; Oc1ccn2cnnc2c1]

According to a modification of General Method 13, 7-bromo[1,2,4]triazolo[4,3-a]pyridine (525 mg, 2.65 mmol) was reacted with 4,4,5,5-tetramethyl-2-(4,4,5,5-tetramethyl-1,3,2-dioxaborolan-2-yl)-1,3,2-dioxaborolane (1.52 g, 5.97 mmol) at 100° C. for 2 h. The mixture was cooled to 0° C. and 30 w % H₂O₂ (542 μL, 5.3 mmol) was added dropwise. The mixture was stirred for 5 h at rt. Work up followed by SCX afforded the product (140 mg, 27% yield).

[M+H]⁺=136.0

¹H NMR (d6-DMSO, 400 MHz) δ 6.61 (1H, dd, J=7.5, 2.1 Hz), 6.73 (1H, d, J+1.8 Hz), 8.38 (1H, d, J=7.3 Hz), 8.98 (1H, s), OH proton not seen

### 1-N-[(2,4-Dimethoxyphenyl)methyl]-5-N-[[5-([1,2,4]triazolo[4,3-a]pyridin-7-yloxymethyl)-2-oxabicyclo[3.1.1]heptan-1-yl]methyl]isoquinoline-1,5-diamine

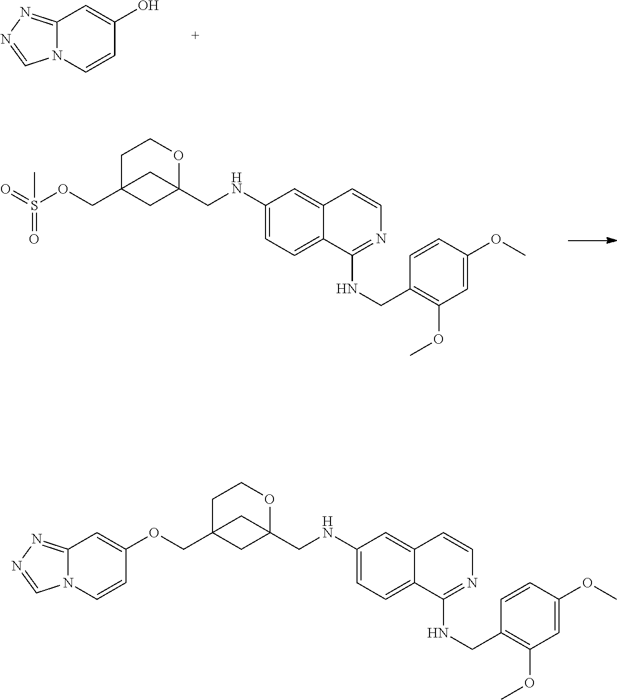 [SMILES: Oc1ccn2cnnc2c1 ; COc1ccc(CNc2nccc3cc(NCC45CC(COS(C)(=O)=O)(CCO4)C5)ccc23)c(OC)c1 ; COc1ccc(CNc2nccc3cc(NCC45CC(COc6ccn7cnnc7c6)(CCO4)C5)ccc23)c(OC)c1]

According to a modification of General Method 2b, to a mixture of [1,2,4]triazolo[4,3-a]pyridin-7-ol (80 mg, 0.41 mmol) and [1-[[[1-[(2,4-dimethoxyphenyl)methylamino]isoquinolin-5-yl]amino]methyl]-2-oxabicyclo[3.1.1]heptan-5-yl]methyl methanesulfonate (219 mg, 0.41 mmol) in DMSO (1 mL) was added K₂CO₃ 325 mesh (172 mg, 1.24 mmol). The mixture was heated under nitrogen at 100° C. for 30 min. The mixture was partitioned between EtOAc (50 mL) and water (25 mL). The organic layer was washed with water (4×20 mL), dried (MgSO₄) and concentrated. The product was purified by flash chromatography (Silica, 0-100% EtOAc in Pet. Ether followed by 0-20% MeOH in EtOAc) to afford the product (120 mg, 46% yield)

[M+H]⁺=567.3

### 5-N-[[5-([1,2,4]Triazolo[4,3-a]pyridin-7-yloxymethyl)-2-oxabicyclo[3.1.1]heptan-1-yl]methyl]isoquinoline-1,5-diamine (Example 10926)

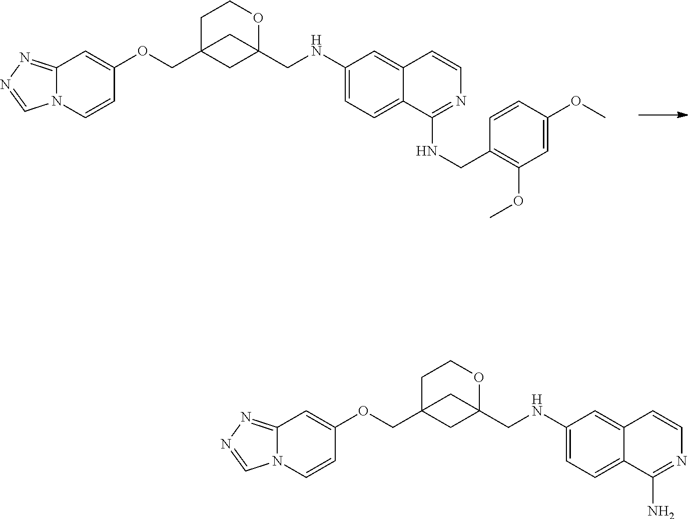 [SMILES: COc1ccc(CNc2nccc3cc(NCC45CC(COc6ccn7cnnc7c6)(CCO4)C5)ccc23)c(OC)c1 ; Nc1nccc2cc(NCC34CC(COc5ccn6cnnc6c5)(CCO3)C4)ccc12]

According to a modification of General Method 8, a solution of 1-N-[(2,4-dimethoxyphenyl)methyl]-5-N-[[5-([1,2,4]triazolo[4,3-a]pyridin-7-yloxymethyl)-2-oxabicyclo[3.1.1]heptan-1-yl]methyl]isoquinoline-1,5-diamine (120 mg, 0.19 mmol) in DCM (1 mL) was treated with TFA (2.70 mL, 35.0 mmol), stirred at rt for 4 h and the mixture concentrated. The residue was taken up in MeOH (2 mL), loaded on to a 2 g SCX-2 column, flushed with MeOH (15 mL), followed by 1M NH₃ in MeOH (15 mL). The latter was concentrated and purified by flash chromatography (Silica, 0-100% EtOAc in Pet. Ether followed by 0-50% MeOH in EtOAc) and lyophilized to afford the product (66 mg, 83% yield).

[M+H]⁺=417.2

¹H (DMSO-d₆, 400 MHz) δ 1.85 (2H, dd, J=7.1, 2.1 Hz), 1.97 (2H, d, J=8.8 Hz), 2.04 (2H, t, J=6.8 Hz), 3.24 (2H, d, J=5.7 Hz), 3.93 (2H, s), 4.10 (2H, t, J=6.8 Hz), 5.78 (1H, t, J=5.8 Hz), 6.52 (2H, s), 6.59 (1H, dd, J=7.4, 2.2 Hz), 6.64 (1H, d, J=7.7 Hz), 7.02 (1H, d, J=2.2 Hz), 7.11 (1H, d, J=6.1 Hz), 7.20 (1H, t, J=8.0 Hz), 7.33 (1H, d, J=8.3 Hz), 7.73 (1H, d, J=6.1 Hz), 8.38 (1H, d, J=7.5 Hz), 8.45 (1H, s)

### Example Number 10928

### 4-((1-(((1-Aminoisoquinolin-5-yl)amino)methyl)-2-oxabicyclo[3.1.1]heptan-5-yl)methoxy)-1,6-dimethylpyridin-2(1H)-one

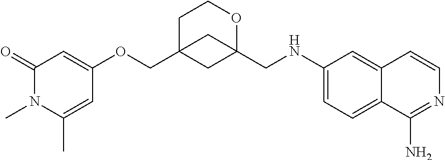 [SMILES: Cc1cc(OCC23CCOC(CNc4ccc5c(N)nccc5c4)(C2)C3)cc(=O)n1C]

### 4-((1-(((1-((2,4-Dimethoxybenzyl)amino)isoquinolin-5-yl)amino)methyl)-2-oxabicyclo[3.1.1]heptan-5-yl)methoxy)-1,6-dimethylpyridin-2(1H)-one

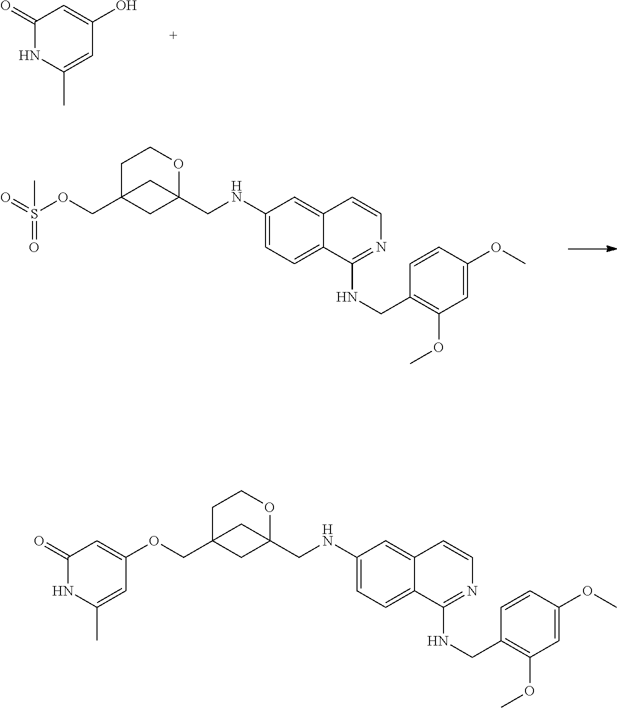

According to a modification of General Method 2b, [1-[[[1-[(2,4-dimethoxyphenyl)methylamino]isoquinolin-5-yl]amino]methyl]-2-oxabicyclo[3.1.1]heptan-5-yl]methyl methanesulfonate, (150 mg, 284 μmol) was dissolved in DMF (5 mL) followed by the addition of 4-hydroxy-1,6-dimethylpyridin-2(1H)-one (59.3 mg, 426 μmol) and K₂CO₃ (118 mg, 853 μmol). The reaction mixture was then heated to 60° C. and stirred for 18 h. The reaction mixture was allowed to cool to rt and was quenched by the addition of water (30 mL). The mixture was then diluted with EtOAc (30 mL) and the aq phase extracted with EtOAc (2×30 mL). The combined organic phase was washed sequentially with sat. aq NaHCO₃, water and Brine (30 mL each) before being dried over MgSO₄, filtered and concentrated. The product was purified by chromatography (Silica, 0-20% (0.7 M NH₃/MeOH)/DCM) to afford the product (50 mg, 28% yield)

¹H NMR (CDCl3, 500 MHz) δ 1.22-1.33 (2H, m), 1.91-1.97 (2H, m), 1.98-2.10 (4H, m), 2.27 (3H, s), 3.27 (2H, s), 3.43 (3H, s), 3.47 (1H, s), 3.79 (3H, s), 3.85 (3H, s), 4.21 (2H, t, J=6.8 Hz), 4.60 (1H, s), 4.74 (2H, d, J=5.2 Hz), 5.77 (2H, d, J=1.5 Hz), 6.44 (1H, dd, J=8.3, 2.4 Hz), 6.49 (1H, d, J=2.4 Hz), 6.68 (1H, d, J=7.7 Hz), 6.87 (1H, d, J=6.2 Hz), 7.07 (1H, d, J=8.4 Hz), 7.28 (1H, t, J=8.0 Hz), 7.32 (1H, d, J=8.2 Hz), 7.99 (1H, d, J=6.2 Hz)

### 4-((1-(((1-Aminoisoquinolin-5-yl)amino)methyl)-2-oxabicyclo[3.1.1]heptan-5-yl)methoxy)-1,6-dimethylpyridin-2(1H)-one (Example 10928)

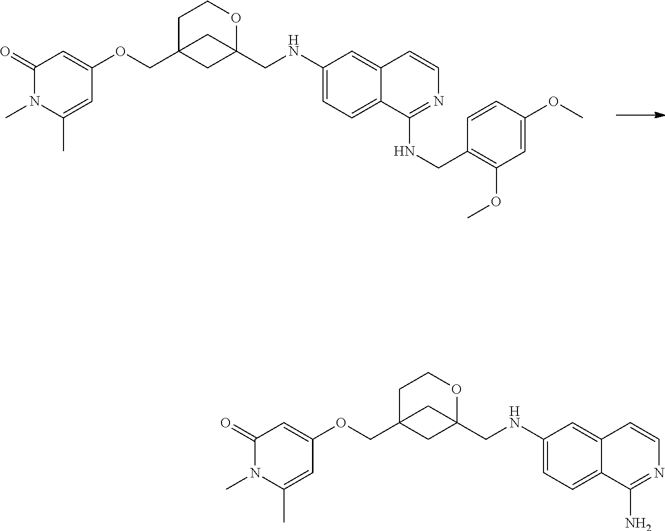

According to a modification of general Method 8, 4-((1-(((1-((2,4-dimethoxybenzyl)amino)isoquinolin-5-yl)amino)methyl)-2-oxabicyclo[3.1.1]heptan-5-yl)methoxy)-1,6-dimethylpyridin-2(1H)-one (50 mg, 88 μmol) was dissolved in DCM (9 mL) followed by the addition of TFA (1 g, 0.01 mol). The reaction mixture was heated to 40° C. and stirred for 1 h and then concentrated. The material was dissolved in MeOH (2 mL) and passed over an SCX column, eluting in 2.5 M NH₃ in MeOH (25 mL). Product-containing eluent was concentrated and lyophilized to afford the product (28 mg, 97% yield).

[M+H]⁺=421.2

¹H NMR (DMSO, 500 MHz) δ 1.75-1.86 (2H, m), 1.91 (2H, dt, J=8.4, 4.2 Hz), 1.96 (2H, t, J=6.9 Hz), 2.25 (3H, s), 3.22 (2H, d, J=5.9 Hz), 3.29 (3H, s), 3.74 (2H, s), 4.06 (2H, t, J=6.8 Hz), 5.64 (1H, d, J=2.8 Hz), 5.73-5.81 (2H, m), 6.64-6.71 (3H, m), 7.13 (1H, d, J=6.3 Hz), 7.23 (1H, t, J=8.0 Hz), 7.35 (1H, d, J=8.3 Hz), 7.71 (1H, d, J=6.2 Hz)

### Example Number 11101

### 4-[[5-[[(1-Aminoisoquinolin-5-yl)amino]methyl]-2-oxabicyclo[3.1.1]heptan-1-yl]methoxy]-1-methylpyridin-2-one

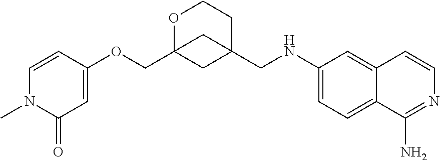 [SMILES: Cn1ccc(OCC23CC(CNc4ccc5c(N)nccc5c4)(CCO2)C3)cc1=O]

### Methyl 5-(hydroxymethyl)-2-oxabicyclo[3.1.1]heptane-1-carboxylate

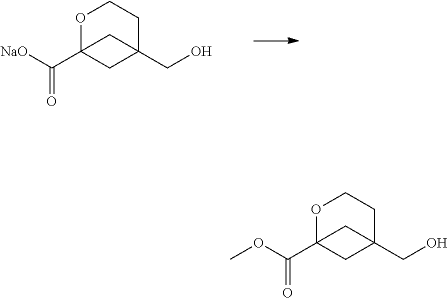

To a stirred suspension of 5-(hydroxymethyl)-2-oxabicyclo[3.1.1]heptane-1-carboxylic acid sodium salt (600 mg, 2.8 mmol) in MeOH (4 mL) in an ice bath was added thionyl chloride (816 μL, 11.19 mmol) dropwise. The reaction was allowed to warm to rt and stirred for 22 h. The reaction mixture was diluted with DCM (40 mL) and washed with water (20 mL). The aq layer was back extracted with DCM (30 mL). The combined organic fractions were dried (Na₂SO₄) and concentrated to afford the product (409 mg, 71% yield).

[M+H]⁺=187.1

¹H NMR (CDCl₃, 400 MHz) δ 1.94-2.05 (4H, m), 2.33 (2H, ddt, J=7.2, 2.3, 1.0 Hz), 3.54 (2H, s), 3.77 (3H, s), 4.19-4.27 (2H, m), OH not seen.

### Methyl 5-(methylsulfonyloxymethyl)-2-oxabicyclo[3.1.1]heptane-1-carboxylate

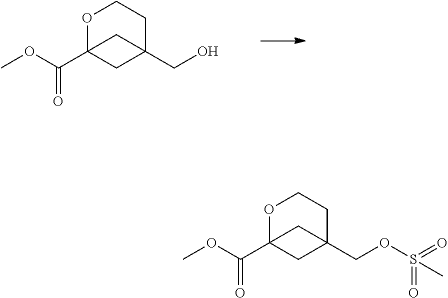

According to a modification of the General Method 1a, a solution of methyl 5-(hydroxymethyl)-2-oxabicyclo[3.1.1]heptane-1-carboxylate (404 mg, 1.95 mmol) and TEA (544 μL, 3.91 mmol) in DCM (5 ml) was cooled to 0° C. and MsCl (227 μL, 2.93 mmol) was added to the mixture and the reaction was allowed to warm to rt and stir for 22 h. The mixture was washed with water (20 mL) and the aq phase was extracted with DCM (30 ml). The combined organic extracts were dried (MgSO₄), filtered and concentrated in to afford the product (372 mg, 58% yield).

¹H NMR (CDCl₃, 400 MHz) δ 2.05-2.13 (4H, m), 2.35-2.44 (2H, m), 3.05 (3H, s), 3.78 (3H, s), 4.12 (2H, s), 4.19-4.26 (2H, m)

### Methyl 5-[(1,3-dioxoisoindol-2-yl)methyl]-2-oxabicyclo[3.1.1]heptane-1-carboxylate

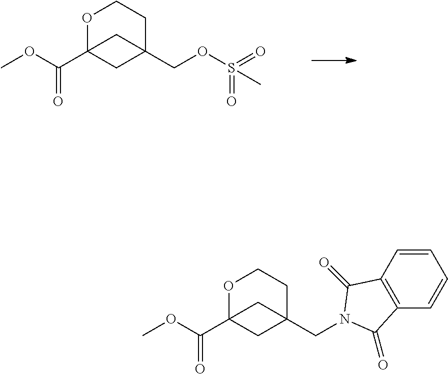

A suspension of methyl 5-(methylsulfonyloxymethyl)-2-oxabicyclo[3.1.1]heptane-1-carboxylate (100 mg, 0.303 mmol) and potassium phthalimide (62.0 mg, 0.333 mmol) in DMF (4 mL) was stirred at 100° C. for 3 h. The mixture was cooled to rt, taken up in water (20 mL) and washed with EtOAc (2×30 mL). The combined organic layers were washed with brine (10 mL), dried (Na₂SO₄) and concentrated to afford the product (98 mg, 82% yield).

[M+H]⁺=316.0

¹H NMR (CDCl₃, 400 MHz) δ 2.01-2.14 (4H, m), 2.30-2.39 (2H, m), 3.73 (5H, s), 4.15-4.21 (2H, m), 7.71-7.79 (2H, m), 7.83-7.90 (2H, m)

### [5-(Aminomethyl)-2-oxabicyclo[3.1.1]heptan-1-yl]methanol

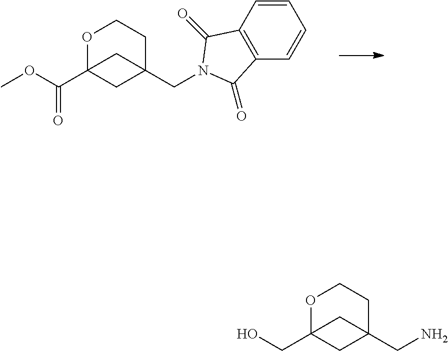 [SMILES: COC(=O)C12CC(CN3C(=O)c4ccccc4C3=O)(CCO1)C2 ; NCC12CCOC(CO)(C1)C2]

To a stirred solution of methyl 5-[(1,3-dioxoisoindol-2-yl)methyl]-2-oxabicyclo[3.1.1]heptane-1-carboxylate (98 mg, 0.25 mmol) in IPA (4 mL) and water (1 mL), was added sodium borohydride (56 mg, 1.49 mmol). After stirring for 24 h, AcOH (256 μL, 4.48 mmol) was added slowly and when the foaming subsided, the reaction was heated to 80° C. for 22 h. The reaction mixture was concentrated, the residue taken up in MeOH (1 mL) and the mixture was passed directly through an SCX and washed with MeOH. The product was eluted with a solution of 1M NH₃ in MeOH and concentrated to afford the product (39 mg, 100% yield).

[M+H]⁺=158.1

¹H NMR (CDCl₃, 400 MHz) δ 1.60-1.67 (2H, m), 1.82 (2H, dd, J=7.2, 2.7 Hz), 1.92-1.98 (2H, m), 2.64 (2H, s), 3.51 (2H, s), 4.07-4.16 (2H, m) three exchangeable protons are not observed.

### [5-[[[1-[(2,4-Dimethoxyphenyl)methylamino]isoquinolin-5-yl]amino]methyl]-2-oxabicyclo[3.1.1]heptan-1-yl]methanol

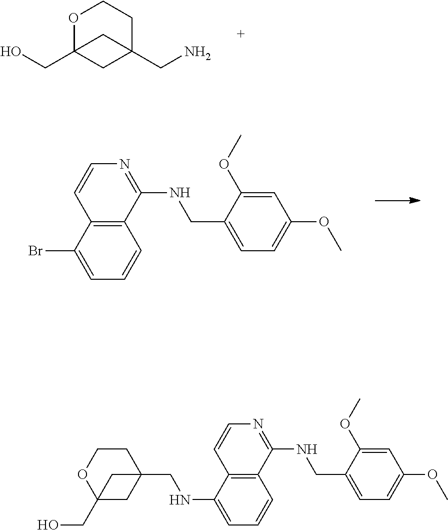 [SMILES: NCC12CCOC(CO)(C1)C2 ; COc1ccc(CNc2nccc3c(Br)cccc23)c(OC)c1 ; COc1ccc(CNc2nccc3c(NCC45CCOC(CO)(C4)C5)cccc23)c(OC)c1]

According to a modification of General Method 4, a suspension of [5-(aminomethyl)-2-oxabicyclo[3.1.1]heptan-1-yl]methanol (40 mg, 0.25 mmol), 5-bromo-N-[(2,4-dimethoxyphenyl)methyl]isoquinolin-1-amine (104 mg, 0.28 mmol) and NaOtBu (37 mg, 0.38 mmol) in 1,4-dioxane (5 ml) was purged with N₂(g) for 5 min. Brettphos Pd G4 (23 mg, 0.03 mmol) was added and the mixture purged for a further 5 min with N₂(g). The mixture was stirred in a sealed vial at 40° C. for 20 h and at 60° C. for another 18 h. The reaction was cooled and concentrated. The residue was purified by flash chromatography (Silica, 0-100% EtOAc in Pet. Ether followed by 0-5% MeOH in EtOAc) to afford the product (23 mg, 20% yield).

[M+H]⁺=450.4

### [5-[[[1-[(2,4-Dimethoxyphenyl)methylamino]isoquinolin-5-yl]amino]methyl]-2-oxabicyclo[3.1.1]heptan-1-yl]methyl methanesulfonate

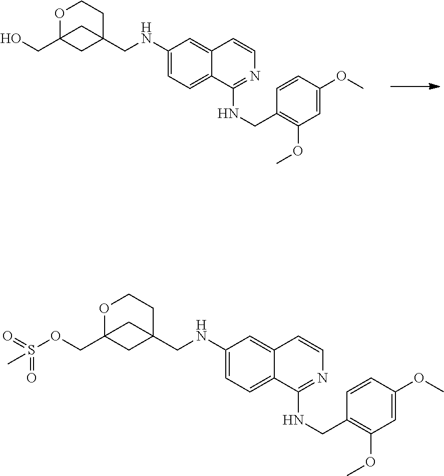 [SMILES: COc1ccc(CNc2nccc3cc(NCC45CCOC(CO)(C4)C5)ccc23)c(OC)c1 ; COc1ccc(CNc2nccc3cc(NCC45CCOC(COS(C)(=O)=O)(C4)C5)ccc23)c(OC)c1]

According to a modification of General Method 1a, a solution of [5-[[[1-[(2,4-dimethoxyphenyl)methylamino]isoquinolin-5-yl]amino]methyl]-2-oxabicyclo[3.1.1]heptan-1-yl]methanol (23 mg, 0.05 mmol) and TEA (14 μL, 0.1 mmol) in DCM (4 mL) was cooled in an ice/water bath. MsCl (8 μL, 0.1 mmol) was added dropwise maintaining the temperature at 0° C. The mixture was stirred 0° C. for 60 min after which time it was allowed to warm to rt and stirred for 3 days. The mixture was quenched with water (30 mL) and washed with DCM (2×50 mL), dried (Na₂SO₄) and concentrated to afford the product (40 mg, 99% yield).

[M+H]⁺=528.5

### 4-[[5-[[[1-[(2,4-Dimethoxyphenyl)methylamino]isoquinolin-5-yl]amino]methyl]-2-oxabicyclo[3.1.1]heptan-1-yl]methoxy]-1-methylpyridin-2-one

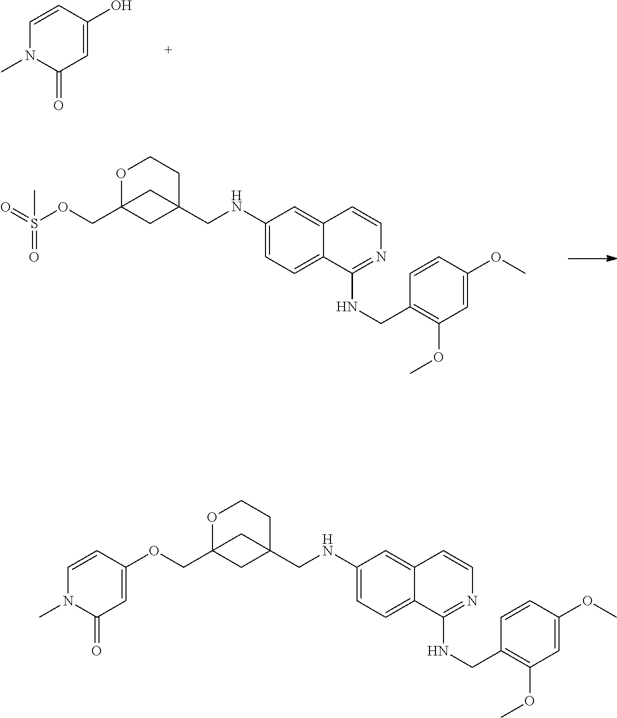

According to a modification of General Method 2b, to a mixture of [5-[[[1-[(2,4-dimethoxyphenyl)methylamino]isoquinolin-5-yl]amino]methyl]-2-oxabicyclo[3.1.1]heptan-1-yl]methyl methanesulfonate (40 mg, 0.05 mmol) and CsCO₃ (50 mg, 0.15 mmol) in MeCN (5 mL) was added 4-hydroxy-1-methyl-2-pyridone (10 mg, 0.08 mmol). The mixture was heated at 60° C. for 20 h. The mixture was cooled, filtered, washed with EtOAc:MeOH 5:1 (20 mL) and the filtrate was concentrated to afford the product (90 mg, 91% yield).

[M+H]⁺=557.6

### 4-[[5-[[(1-Aminoisoquinolin-5-yl)amino]methyl]-2-oxabicyclo[3.1.1]heptan-1-yl]methoxy]-1-methylpyridin-2-one (Example 11101)

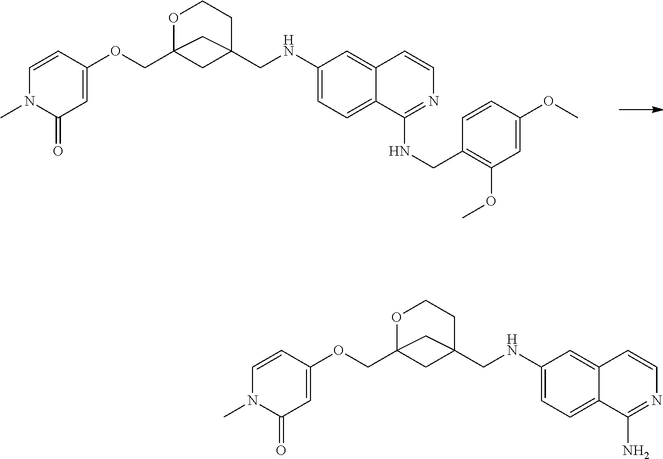

According to a modification of General Method 8, 4-[[5-[[[1-[(2,4-dimethoxyphenyl)methylamino]isoquinolin-5-yl]amino]methyl]-2-oxabicyclo[3.1.1]heptan-1-yl]methoxy]-1-methylpyridin-2-one (90 mg, 0.06 mmol) was taken up in TFA (990 μL, 12.9 mmol) and stirred at 40° C. for 30 min. After removal of TFA, the residue was suspended in MeOH (2 ml) and loaded on to a 2 g SCX-2 column, which was flushed with MeOH (10 mL), followed by 3.5 M NH₃ in MeOH (15 mL) to elute the product. Purification by automated prep HPLC (mass directed 2-60% over 20 min in basic mobile phase) afforded the product (5 mg, 19% yield).

[M+H]⁺=407.4

¹H NMR (DMSO-d₆, 400 MHz) δ 1.77-1.84 (2H, m), 1.87-1.95 (2H, m), 2.02 (2H, t, J=6.9 Hz), 3.18 (2H, d, J=5.8 Hz), 3.30 (3H, s), 3.79 (2H, s), 4.02 (2H, t, J=6.8 Hz), 5.72 (1H, d, J=2.8 Hz), 5.85 (1H, dd, J=7.5, 2.7 Hz), 5.89 (1H, t, J=6.0 Hz), 6.45-6.52 (2H, m), 6.61 (1H, d, J=7.7 Hz), 7.16-7.22 (2H, m), 7.31 (1H, d, J=8.3 Hz), 7.51 (1H, d, J=7.5 Hz), 7.72 (1H, d, J=6.1 Hz)

### Example Number 12014

### 4-((1-(((1-Aminoisoquinolin-5-yl)amino)methyl)-2-(phenethylsulfonyl)-2-azabicyclo[2.1.1]hexan-4-yl)methoxy)-1-methylpyridin-2(1H)-one

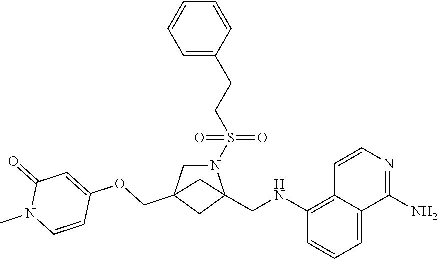 [SMILES: Cn1ccc(OCC23CN(S(=O)(=O)CCc4ccccc4)C(CNc4cccc5c(N)nccc45)(C2)C3)cc1=O]

### 4-[[1-[[(1-aminoisoquinolin-5-yl)amino]methyl]-2-(2-phenylethylsulfonyl)-2-azabicyclo[2.1.1]hexan-4-yl]methoxy]-1-methylpyridin-2-one (Example 12014)

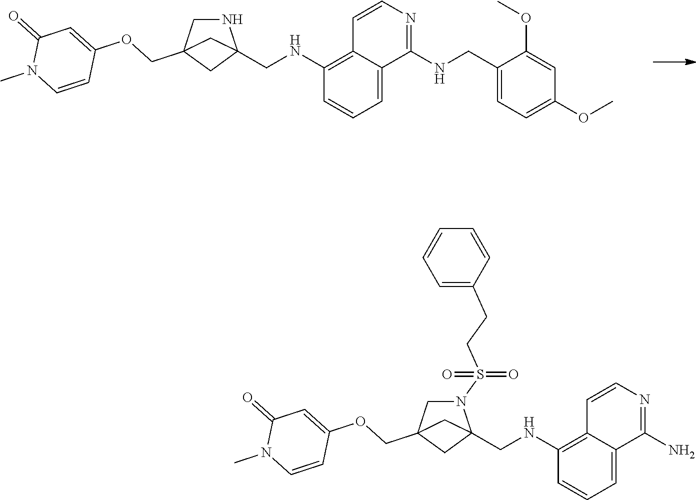

To a solution of 4-[[4-[[[1-[(2,4-dimethoxyphenyl)methylamino]-5-isoquinolyl]amino]methyl]-2-azabicyclo[2.1.1]hexan-1-yl]methoxy]-1-methyl-pyridin-2-one (68 mg, 0.126 mmol) and TEA (40 μL, 0.289 mmol) in anhydrous DCM (2 mL) at 0° C. was added 2-phenylethanesulfonyl chloride (51.4 mg, 0.251 mmol). The mixture was stirred at 0° C. for 2 h and concentrated. The residue was taken up in in anhydrous DCM (2 mL) at rt and according to a variation of General Method 8, TFA (811 μL, 10.9 mmol) was added. The mixture was stirred at rt for 2.5 h and concentrated. The residue was purified by flash chromatography (Silica-C18, 0-80% MeCN in water [10 mM NH₄HCO₃]) followed by automated prep HPLC (mass directed, 35-45% MeCN in water [10 mM NH₄HCO₃]) and lyophilized to afford the product.

[M+H]⁺=560.2

¹H NMR (500 MHz, DMSO-d6) δ 1.65 (2H, dd, J=4.8, 1.9 Hz), 1.86-1.93 (2H, m), 3.04-3.11 (2H, m), 3.31 (3H, s), 3.45-3.49 (4H, m), 3.81 (2h, d, J=6.1 Hz), 4.14 (2H, s), 5.78 (1H, d, J=2.7 Hz), 5.86-5.93 (2h, m), 6.53 (2h, d, J=5.3 Hz), 6.69 (1H, d, J=7.7 Hz), 6.99-7.04 (1h, m), 7.22-7.29 (6H, m), 7.37 (1H, d, J=8.3 Hz), 7.53 (1H, d, J=7.6 Hz), 7.74 (1H, d, J=6.1 Hz)

### Example Number 12019

### 4-((1-(((1-Aminoisoquinolin-5-yl)amino)methyl)-2-(3-phenylpropyl)-2-azabicyclo[2.1.1]hexan-4-yl)methoxy)-1-methylpyridin-2(1H)-one

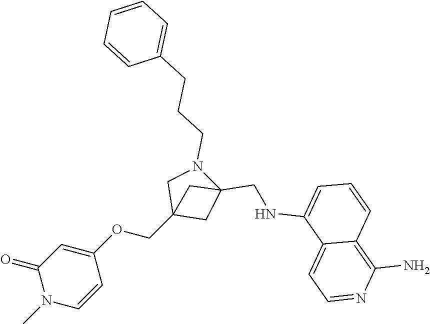 [SMILES: Cn1ccc(OCC23CN(CCCc4ccccc4)C(CNc4cccc5c(N)nccc45)(C2)C3)cc1=O]

### 4-((1-(((1-((2,4-Dimethoxybenzyl)amino)isoquinolin-5-yl)amino)methyl)-2-(3-phenylpropyl)-2-azabicyclo[2.1.1]hexan-4-yl)methoxy)-1-methylpyridin-2(1H)-one

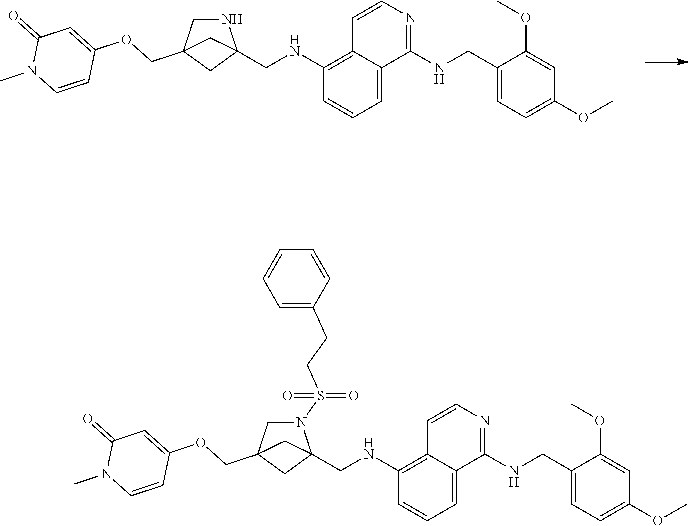

3-Phenylpropanal (22 mg, 0.16 mmol) was added to a mixture of 4-[[4-[[[1-[(2,4-dimethoxyphenyl)methylamino]-5-isoquinolyl]amino]methyl]-2-azabicyclo[2.1.1]hexan-1-yl]methoxy]-1-methyl-pyridin-2-one (75 mg, 0.14 mmol) and AcOH (12 μL, 0.21 mmol) in dichloroethane (1.5 mL). The mixture was stirred at rt for 30 min then STAB (73 mg, 0.34 mmol) was added. The mixture was stirred at rt for 30 min. The reaction was quenched with sat. NaHCO₃ (aq) (5 mL) and extracted with DCM (3×2 mL). The combined organic layers were dried (Na₂SO₄), filtered and concentrated. Flash chromatography (Silica, 0-40% MeOH in EtOAc) afforded the product (79 mg, 87% yield).

[M+H]⁺=660.5

### 4-((1-(((1-Aminoisoquinolin-5-yl)amino)methyl)-2-(3-phenylpropyl)-2-azabicyclo[2.1.1]hexan-4-yl)methoxy)-1-methylpyridin-2(1H)-one (Example 12019)

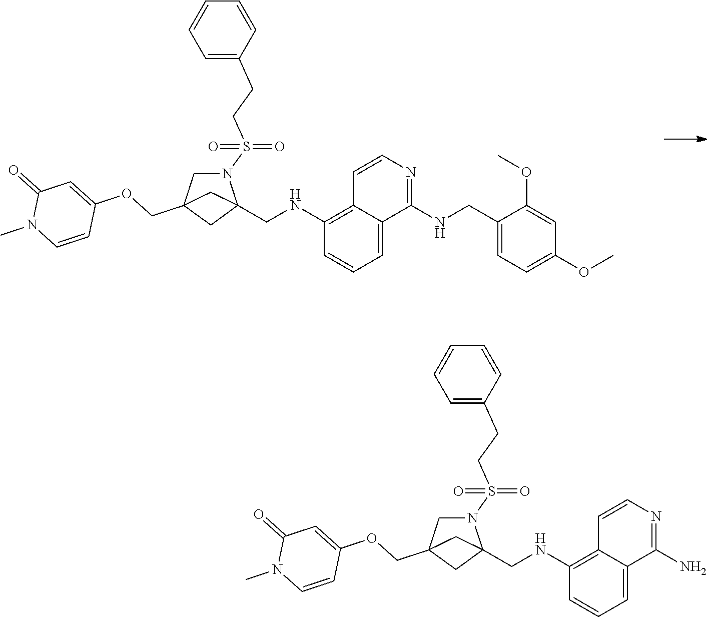

4-((1-(((1-((2,4-Dimethoxybenzyl)amino)isoquinolin-5-yl)amino)methyl)-2-(3-phenylpropyl)-2-azabicyclo[2.1.1]hexan-4-yl)methoxy)-1-methylpyridin-2(1H)-one (79 mg, 0.120 mmol) was deprotected according to General Method 8 in TFA (178 μL, 2.39 mmol) and DCM (1200 μL) at rt for 1 h. The crude product was purified by flash chromatography (Silica-C18, 10-30% MeCN in water [10 mM NH₄HCO₃]). The product was loaded onto a PL-HCO₃ MP SPE cartridge (250 mg) eluting with a mixture of MeOH and MeCN (1:1, 5 mL). The filtrate was concentrated, and lyophilisation afforded the product (16 mg, 26%).

[M+H]⁺=510.4

¹H NMR (DMSO-d6, 500 MHz) δ 1.61 (4H, q, J=5.9 Hz), 1.72 (2H, p, J=7.1 Hz), 2.52 (2H, d, J=5.7 Hz), 2.64-2.68 (2H, m), 2.72 (2H, s), 3.31 (3H, s), 3.36 (2H, d, J=5.2 Hz), 4.09 (2H, s), 5.58 (1H, t, J=5.3 Hz), 5.76 (1H, d, J=2.8 Hz), 5.89 (1H, dd, J=7.5, 2.8 Hz), 6.52 (2H, s), 6.57 (1H, d, J=7.7 Hz), 6.98 (1H, d, J=5.9 Hz), 7.09-7.14 (1H, m), 7.15-7.24 (5H, m), 7.35 (1H, d, J=8.4 Hz), 7.52 (1H, d, J=7.6 Hz), 7.73 (1H, d, J=6.1 Hz).

### Example Number 12034

### 4-((1-(((1-Aminoisoquinolin-5-yl)amino)methyl)-2-(2-(benzofuran-5-yl)acetyl)-2-azabicyclo[2.1.1]hexan-4-yl)methoxy)-1-methylpyridin-2(1H)-one

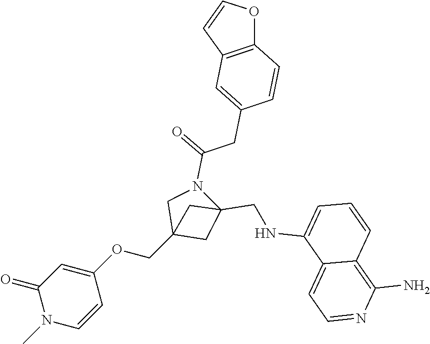 [SMILES: Cn1ccc(OCC23CN(C(=O)Cc4ccc5occc5c4)C(CNc4cccc5c(N)nccc45)(C2)C3)cc1=O]

### 4-[[2-[2-(Benzofuran-5-yl)acetyl]-1-[[[1-[(2,4-dimethoxyphenyl)methylamino]-5-isoquinolyl]amino]methyl]-2-azabicyclo[2.1.1]hexan-4-yl]methoxy]-1-methyl-pyridin-2-one

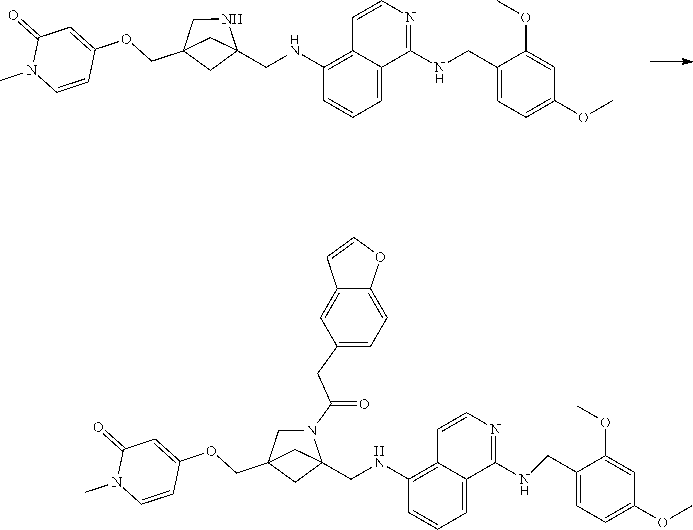

To a solution of 4-[[4-[[[1-[(2,4-dimethoxyphenyl)methylamino]-5-isoquinolyl]amino]methyl]-2-azabicyclo[2.1.1]hexan-1-yl]methoxy]-1-methyl-pyridin-2-one (100 mg, 0.185 mmol) in DCM (2 mL) were added 2-(benzofuran-5-yl)acetic acid (32.5 mg, 0.185 mmol), HATU (77.2 mg, 0.203 mmol) and DIPEA (104 μL, 0.609 mmol). The mixture was stirred at rt for 16 h then diluted with DCM (10 mL), washed with water (3×10 mL), dried (MgSO₄) and concentrated to afford the product.

[M+H]+=700.0

### 4-((1-(((1-Aminoisoquinolin-5-yl)amino)methyl)-2-(2-(benzofuran-5-yl)acetyl)-2-azabicyclo[2.1.1]hexan-4-yl)methoxy)-1-methylpyridin-2(1H)-one (Example 12034)

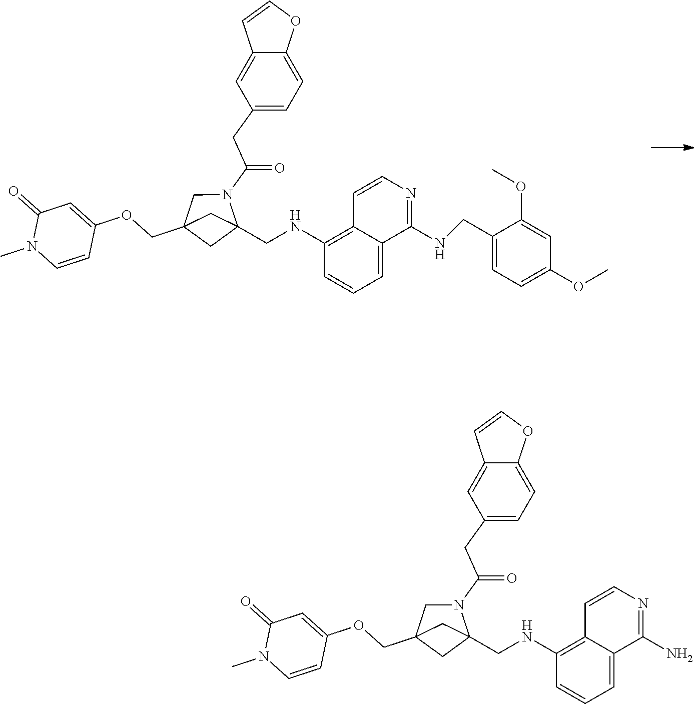

4-[[2-[2-(Benzofuran-5-yl)acetyl]-1-[[[1-[(2,4-dimethoxyphenyl)methylamino]-5-isoquinolyl]amino]methyl]-2-azabicyclo[2.1.1]hexan-4-yl]methoxy]-1-methylpyridin-2-one was deprotected according to General Method 8 in anhydrous DCM (2 mL) and TFA (1.2 mL, 16.1 mmol) at rt for 16 h. The mixture was concentrated and diluted with NaOH(aq) (4M, 10 mL) and extracted with a mixture of CHCl₃ and IPA (3:1, 3×10 mL). The organic layers were combined, washed with brine (15 mL), dried (Na₂SO₄) and concentrated. The product was purified by automated prep HPLC (mass directed, 32-42% MeCN in water [10 mM NH₄HCO₃]) and lyophilized to afford the product (57 mg, 56% over 2 steps) as a solid.

[M+H]⁺=550.3

¹H NMR (DMSO-d6, 500 MHz) δ 1.50 (2H, d, J=4.4 Hz), 1.90 (2H, d, J=4.7 Hz), 3.30 (3H, s), 3.60 (2H, s), 3.74 (2H, s), 3.99 (2H, d, J=6.5 Hz), 4.13 (2H, s), 5.77 (1H, d, J=2.8 Hz), 5.87 (1H, dd, J=7.5, 2.8 Hz), 6.16 (1H, t, J=6.7 Hz), 6.53 (2H, s), 6.78 (2H, dd, J=13.3, 7.0 Hz), 6.90 (1H, dd, J=2.2, 1.0 Hz), 7.19 (1H, t, J=7.9 Hz), 7.25 (1H, dd, J=8.4, 1.8 Hz), 7.32 (1H, d, J=8.3 Hz), 7.50-7.55 (2H, m), 7.57 (1H, d, J=1.8 Hz), 7.67 (1H, d, J=6.1 Hz), 7.97 (1H, d, J=2.2 Hz).

### Example Number 12041

### 1-[[(1-Aminoisoquinolin-5-yl)amino]methyl]-N-benzyl-N-methyl-4-[(1-methyl-2-oxopyridin-4-yl)oxymethyl]-2-azabicyclo[2.1.1]hexane-2-carboxamide

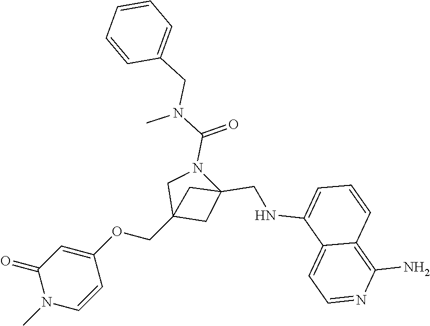 [SMILES: CN(Cc1ccccc1)C(=O)N1CC2(COc3ccn(C)c(=O)c3)CC1(CNc1cccc3c(N)nccc13)C2]

### N-Benzyl-1-[[[1-[(2,4-dimethoxyphenyl)methylamino]-5-isoquinolyl]amino]methyl]-N-methyl-4-[(1-methyl-2-oxo-4-pyridyl)oxymethyl]-2-azabicyclo[2.1.1]hexane-2-carboxamide

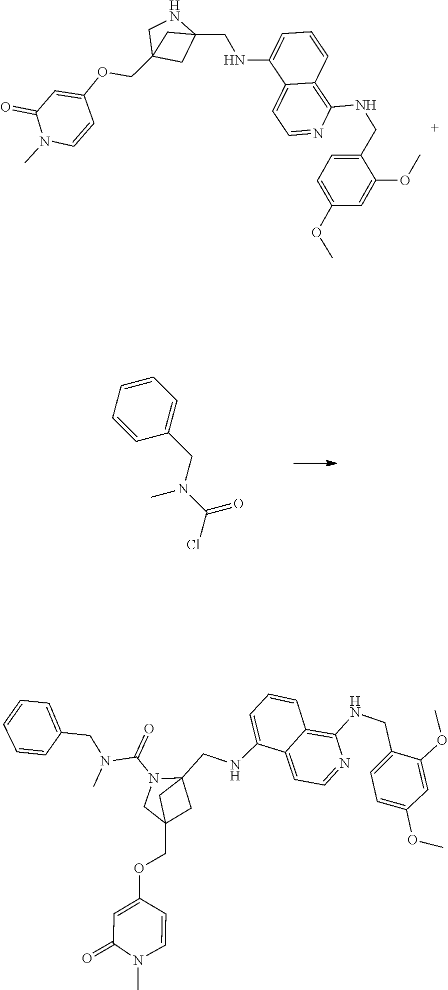 [SMILES: COc1ccc(CNc2nccc3c(NCC45CC(COc6ccn(C)c(=O)c6)(CN4)C5)cccc23)c(OC)c1 ; CN(Cc1ccccc1)C(=O)Cl ; COc1ccc(CNc2nccc3c(NCC45CC(COc6ccn(C)c(=O)c6)(CN4C(=O)N(C)Cc4ccccc4)C5)cccc23)c(OC)c1]

A mixture of N-methyl-1-phenyl-methanamine (1300 μL, 9.77 mmol) and DIPEA (3400 μL, 19.8 mmol) in anhydrous DCM (20 mL) was added over 30 min to a solution of phosgene in toluene (20 wt %, 12 mL, 25.8 mmol) at 0° C. The mixture was stirred at 0° C. for 3 h and concentrated. The residue was triturated with Et₂O (20 mL). The solid was filtered, washed with Et₂O (3×20 mL) and the filtrate was concentrated to afford N-benzyl-N-methyl-carbamoyl chloride (2136 mg). N-Benzyl-N-methyl-carbamoyl chloride (37.2 mg, 0.162 mmol) in anhydrous THF (1 mL) was added to a mixture of 4-[[4-[[[1-[(2,4-dimethoxyphenyl)methylamino]-5-isoquinolyl]amino]methyl]-2-azabicyclo[2.1.1]hexan-1-yl]methoxy]-1-methyl-pyridin-2-one (79.3 mg, 0.139 mmol) and TEA (50 μL, 0.36 mmol) in THF (1 mL) at rt. The mixture was stirred for 18 h and additional N-benzyl-N methyl-carbamoyl chloride (23 mg, 0.104 mmol) in anhydrous THF (500 μL) was added at rt. The mixture was stirred at rt for 18 h and concentrated to provide the product as a solid.

[M+H]⁺=689.2

### 1-[[(1-Amino-5-isoquinolyl)amino]methyl]-N-benzyl-N-methyl-4-[(1-methyl-2-oxo-4-pyridyl)oxymethyl]-2-azabicyclo[2.1.1]hexane-2-carboxamide (Example 12041)

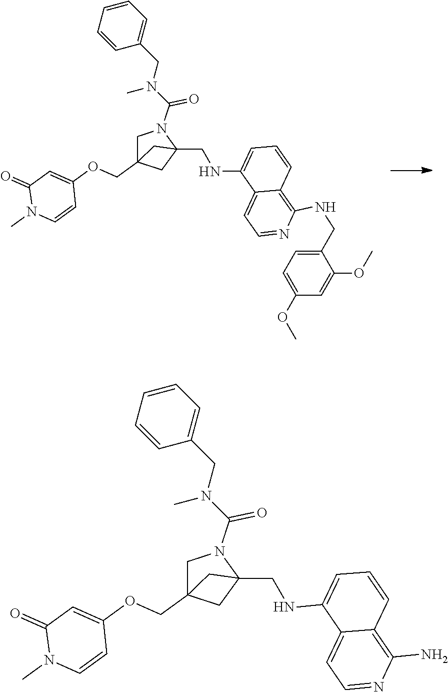 [SMILES: COc1ccc(CNc2nccc3c(NCC45CC(COc6ccn(C)c(=O)c6)(CN4C(=O)N(C)Cc4ccccc4)C5)cccc23)c(OC)c1 ; CN(Cc1ccccc1)C(=O)N1CC2(COc3ccn(C)c(=O)c3)CC1(CNc1cccc3c(N)nccc13)C2]

N-Benzyl-1-[[[1-[(2,4-dimethoxyphenyl)methylamino]-5-isoquinolyl]amino]methyl]-N-methyl-4-[(1-methyl-2-oxo-4-pyridyl)oxymethyl]-2-azabicyclo[2.1.1]hexane-2-carboxamide was deprotected according to General Method 8 in DCM (750 μL) and TFA (750 μL, 10.0 mmol) at rt. The mixture was stirred for 3 h and diluted with NaOH(aq) (2M, 10 mL). The mixture was stirred for 10 min and the aq. phase was extracted with a mixture of DCM and MeOH (9:1 v/v, 3×20 mL). The combined organic phases were dried (Na₂SO₄), filtered and concentrated. The residue was diluted with a mixture of MeOH and MeCN (1:1 v/v, 2 mL) and loaded onto a PL-HCO₃ MP SPE cartridge (200 mg). The cartridge was washed with a mixture of MeOH and MeCN (1:1 v/v, 3×2 mL) and the filtrate was concentrated. The residue was purified by flash chromatography (Silica-C18, 25-50% MeCN in water [10 mM NH₄HCO₃]) and lyophilized to provide the product (42.7 mg, 57% yield over two steps) as a solid.

[M+H]⁺=539.4.

¹H NMR (DMSO-d6, 400 MHz) δ 1.57-1.64 (2H, m), 1.72-1.79 (2H, m), 2.82 (3H, s), 3.30 (3H, s), 3.30 (2H, s), 3.85 (2H, d, J=6.1 Hz), 4.12 (2H, s), 4.50 (2H, s), 5.74 (1H, d, J=2.8 Hz), 5.87 (1H, dd, J=7.5, 2.8 Hz), 6.16 (1H, t, J=6.2 Hz), 6.47 (2H, s), 6.74 (1H, d, J=7.8 Hz), 7.06-7.11 (1H, m), 7.18 (1H, t, J=8.0 Hz), 7.23-7.39 (6H, m), 7.51 (1H, d, J=7.5 Hz), 7.71 (1H, d, J=6.1 Hz).

### Example Number 12057

### 4-((1-(((1-Aminoisoquinolin-5-yl)amino)methyl)-2-(benzo[d]oxazol-2-yl)-2-azabicyclo[2.1.1]hexan-4-yl)methoxy)-1-methylpyridin-2(1H)-one

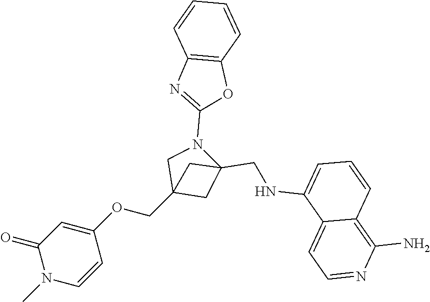 [SMILES: Cn1ccc(OCC23CN(c4nc5ccccc5o4)C(CNc4cccc5c(N)nccc45)(C2)C3)cc1=O]

### 4-[[2-(1,3-Benzoxazol-2-yl)-1-[[[1-[(2,4-dimethoxyphenyl)methylamino]-5-isoquinolyl]amino]methyl]-2-azabicyclo[2.1.1]hexan-4-yl]methoxy]-1-methyl-pyridin-2-one

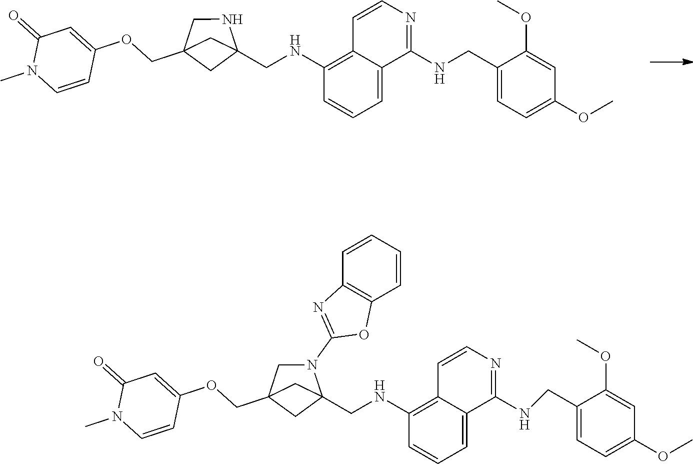

A mixture of 4-[[4-[[[1-[(2,4-dimethoxyphenyl)methylamino]-5-isoquinolyl]amino]methyl]-2-azabicyclo[2.1.1]hexan-1-yl]methoxy]-1-methyl-pyridin-2-one (75 mg, 0.138 mmol), 2-chloro-1,3-benzoxazole (24 mg, 0.156 mmol) and K₂CO₃ (60 mg, 0.434 mmol) in anhydrous DMSO (1 mL) was heated to 140° C. and stirred for 72 h. The mixture was cooled to rt and was quenched with brine (20 mL). The aqueous phase was extracted with EtOAc (3×25 mL) and the combined organic layers were washed with brine (2×20 mL), dried (Na₂SO₄), filtered and concentrated. Flash chromatography (Silica, 40% MeOH in DCM) afforded the product (66.7 mg, 70% yield).

[M+H]⁺=659.4

¹H NMR (DMSO-d6, 400 MHz) δ 1.67 (2H, dd, J=4.7, 1.8 Hz), 1.95-1.99 (2H, m), 3.27 (3H, s), 3.66 (5H, s), 3.78 (3H, s), 4.12-4.23 (4H, m), 4.54 (2H, d, J=5.7 Hz), 5.75 (1H, d, J=2.8 Hz), 5.86 (1H, dd, J=7.5, 2.8 Hz), 6.34 (1H, dd, J=8.4, 2.4 Hz), 6.46-6.53 (2H, m), 6.80 (1H, d, J=7.8 Hz), 6.95-6.99 (3H, m), 7.02 (1H, td, J=7.8, 1.3 Hz), 7.14 (1H, td, J=7.7, 1.1 Hz), 7.22 (1H, t, J=8.0 Hz), 7.31-7.44 (4H, m), 7.49 (1H, d, J=7.6 Hz), 7.66 (1H, d, J=6.1 Hz)

### 4-((1-(((1-Aminoisoquinolin-5-yl)amino)methyl)-2-(benzo[d]oxazol-2-yl)-2-azabicyclo[2.1.1]hexan-4-yl)methoxy)-1-methylpyridin-2(1H)-one (Example 12057)

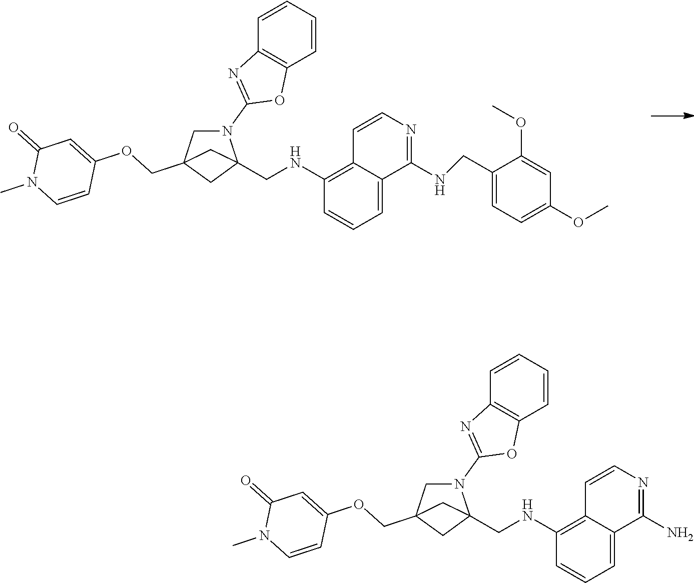

4-[[2-(1,3-Benzoxazol-2-yl)-1-[[[1-[(2,4-dimethoxyphenyl)methylamino]-5-isoquinolyl]amino]methyl]-2-azabicyclo[2.1.1]hexan-4-yl]methoxy]-1-methylpyridin-2-one (66.7 mg, 0.0962 mmol) was deprotected according to General Method 8 in anhydrous DCM (1 mL) and TFA (300 μL, 4.04 mmol) at rt for 18 h. The mixture was quenched with 1M NaOH(aq) (25 mL) and DCM (25 mL). The layers were separated and the aqueous layer was extracted with 1:3 IPA:CHCl₃ (2×20 mL). The combined organic layers were washed with brine (25 mL), dried (Na₂SO₄), filtered and concentrated. The product was purified by flash chromatography (Silica-C18, 0-75% MeCN in water [10 mM NH₄HCO₃]) and lyophilised to afford the product (27.3 mg, 56% yield).

[M+H]⁺=509.2

¹H NMR (DMSO-d6, 400 MHz) δ 1.67 (2H, dd, J=4.7, 1.8 Hz), 1.97 (2H, d, J=4.8 Hz), 3.27 (3H, s), 3.66 (2H, s), 4.11-4.21 (4H, m), 5.76 (1H, d, J=2.8 Hz), 5.86 (1H, dd, J=7.5, 2.8 Hz), 6.47 (3H, s), 6.77 (1H, d, J=7.7 Hz), 6.97-7.00 (1H, m), 7.02 (1H, td, J=7.7, 1.3 Hz), 7.13-7.17 (1H, m), 7.20 (1H, d, J=7.9 Hz), 7.30 (1H, d, J=8.4 Hz), 7.32-7.35 (1H, m), 7.39 (1H, ddd, J=7.9, 1.2, 0.6 Hz), 7.49 (1H, d, J=7.6 Hz), 7.66 (1H, d, J=6.0 Hz)

### Example Number 12060

### 4-((1-(((1-Aminoisoquinolin-5-yl)amino)methyl)-2-(5-phenylpyrimidin-2-yl)-2-azabicyclo[2.1.1]hexan-4-yl)methoxy)-1-methylpyridin-2(1H)-one

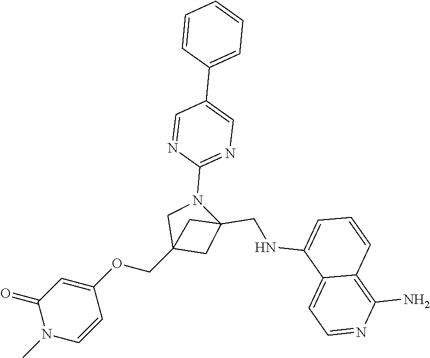 [SMILES: Cn1ccc(OCC23CN(c4ncc(-c5ccccc5)cn4)C(CNc4cccc5c(N)nccc45)(C2)C3)cc1=O]

### 4-[[1-[[[1-[(2,4-Dimethoxyphenyl)methylamino]-5-isoquinolyl]amino]methyl]-2-(5-phenylpyrimidin-2-yl)-2-azabicyclo[2.1.1]hexan-4-yl]methoxy]-1-methyl-pyridin-2-one

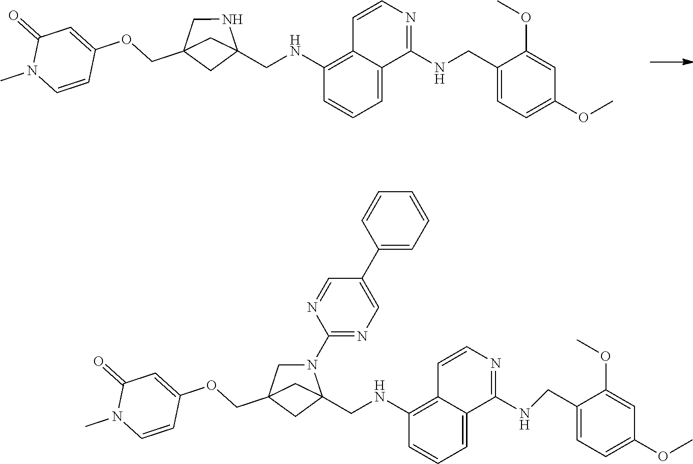

DIPEA (23 μL, 0.164 mmol) was added to a mixture of 4-[[4-[[[1-[(2,4-dimethoxyphenyl)methylamino]-5-isoquinolyl]amino]methyl]-2-azabicyclo[2.1.1]hexan-1-yl]methoxy]-1-methyl-pyridin-2-one (60 mg, 0.111 mmol) and 2-chloro-5-phenyl-pyrimidine (22 mg, 0.115 mmol) in DMF (1000 μL) at rt. The mixture was stirred at 100° C. for 48 h then concentrated. The product was purified by flash chromatography (Silica, 0-40% MeOH in EtOAc to afford the product (61 mg, 79% yield).

[M+H]⁺=696.4

### 4-(1-(((1-Aminoisoquinolin-5-yl)amino)methyl)-2-(5-phenylpyrimidin-2-yl)-2-azabicyclo[2.1.1]hexan-4-yl)methoxy)-1-methylpyridin-2(1H)-one (Example 12060)

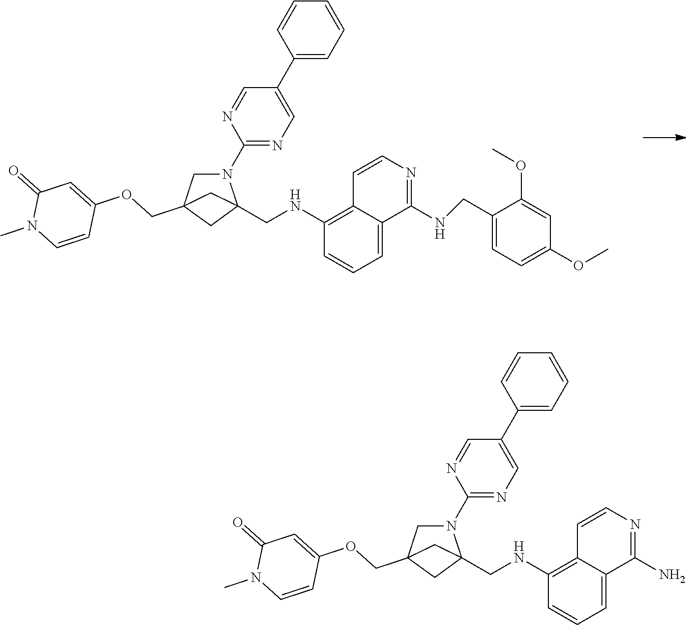

4-[[1-[[[1-[(2,4-Dimethoxyphenyl)methylamino]-5-isoquinolyl]amino]methyl]-2-(5-phenylpyrimidin-2-yl)-2-azabicyclo[2.1.1]hexan-4-yl]methoxy]-1-methyl-pyridin-2-one (61 mg, 0.0877 mmol) was deprotected according to General Method 8 in DCM (1000 μL) and TFA (130 μL, 1.75 mmol) at rt for 18 h then concentrated. The residue was loaded onto a PL-HCO₃ MP SPE cartridge eluting with a mixture of MeCN and MeOH (1:1 v/v, 3×2 mL). The product was purified by flash chromatography (C18, 5-80% MeCN in water [10 mM NH₄HCO₃]) then lyophilized to afford the product (33 mg, 69% yield).

[M+H]⁺=546.3

¹H NMR (DMSO-d6, 400 MHz) δ 1.68 (2H, d, J=3.0 Hz), 2.02 (2H, d, J=4.3 Hz), 3.31 (3H, s), 3.61 (2H, s), 4.22 (2H, s), 4.26 (2H, d, J=6.4 Hz), 5.81 (1H, d, J=2.8 Hz), 5.91 (1H, dd, J=7.5, 2.8 Hz), 6.31 (1H, t, J=6.4 Hz), 6.50 (2H, s), 6.81 (1H, d, J=7.7 Hz), 6.96 (1H, d, J=6.2 Hz), 7.23 (1H, t, J=8.0 Hz), 7.30-7.38 (2H, m), 7.46 (2H, t, J=7.7 Hz), 7.54 (1H, d, J=7.6 Hz), 7.64-7.73 (3H, m), 8.75 (2H, s)

### Example Number 12061

### 1-(((1-Aminoisoquinolin-5-yl)amino)methyl)-N-(4-methoxyphenyl)-4-(((1-methyl-2-oxo-1,2-dihydropyridin-4-yl)oxy)methyl)-2-azabicyclo[2.1.1]hexane-2-carboxamide

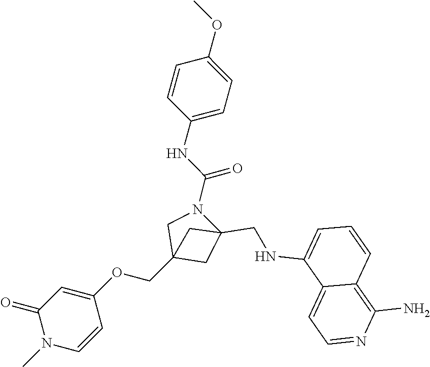 [SMILES: COc1ccc(NC(=O)N2CC3(COc4ccn(C)c(=O)c4)CC2(CNc2cccc4c(N)nccc24)C3)cc1]

### 1-[[[1-[(2,4-Dimethoxyphenyl)methylamino]-5-isoquinolyl]amino]methyl]-N-(4-methoxyphenyl)-4-[(1-methyl-2-oxo-4-pyridyl)oxymethyl]-2-azabicyclo[2.1.1]hexane-2-carboxamide

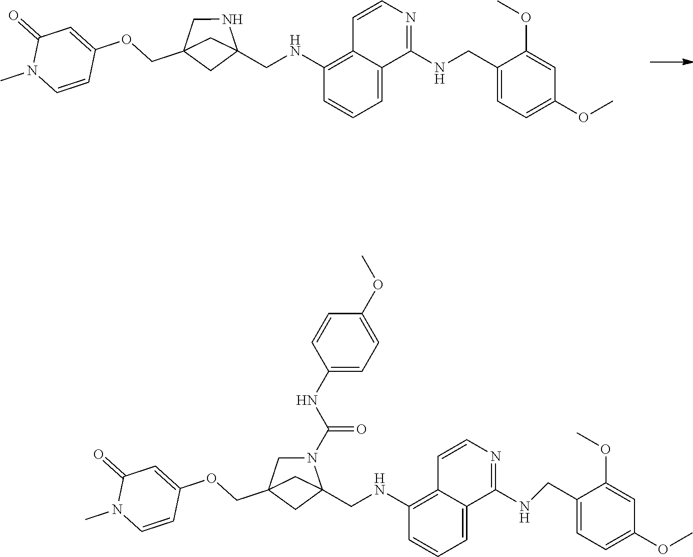

TEA (48 μL, 0.34 mmol) was added to a mixture of 4-[[4-[[[1-[(2,4-dimethoxyphenyl)methylamino]-5-isoquinolyl]amino]methyl]-2-azabicyclo[2.1.1]hexan-1-yl]methoxy]-1-methyl-pyridin-2-one (60 mg, 0.11 mmol) and 1-(isocyanatomethyl)-4-methoxy-benzene (17 μL, 0.12 mmol) in THF (1 mL) at rt. The mixture was stirred at rt for 30 min then concentrated to afford the product (101 mg).

[M+H]⁺=691.4

### 1-(((1-Aminoisoquinolin-5-yl)amino)methyl)-N-(4-methoxyphenyl)-4-(((1-methyl-2-oxo-1,2-dihydropyridin-4-yl)oxy)methyl)-2-azabicyclo[2.1.1]hexane-2-carboxamide (Example 12061)

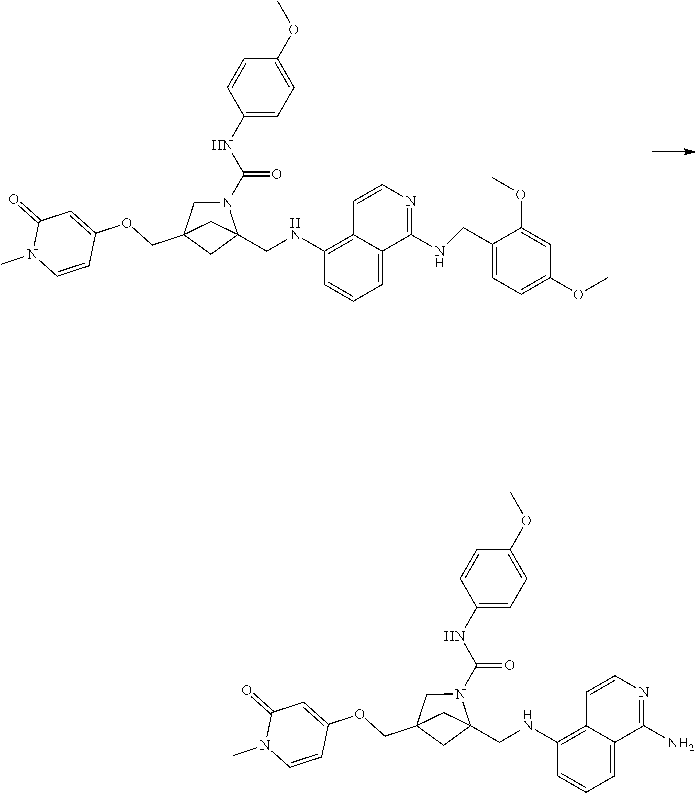

1-[[[1-[(2,4-Dimethoxyphenyl)methylamino]-5-isoquinolyl]amino]methyl]-N-(4-methoxyphenyl)-4-[(1-methyl-2-oxo-4-pyridyl)oxymethyl]-2-azabicyclo[2.1.1]hexane-2-carboxamide (101 mg) was deprotected according to General Method 8 in DCM (1.0 mL) and TFA (165 μL, 2.22 mL) at rt for 18 h then concentrated. The material was loaded onto a PL-HCO₃ MP SPE cartridge eluting with a mixture of MeCN and MeOH (1:1 v/v, 3×2 mL). The crude product was purified by flash chromatography (C18, 5-65% MeCN in water [10 mM NH₄HCO₃ then lyophilized to afford the product (36 mg, 60% yield).

[M+H]⁺=541.3

¹H NMR (DMSO-d6, 400 MHz) δ 1.57 (2H, d, J=3.0 Hz), 1.85 (2H, d, J=4.2 Hz), 3.31 (3H, s), 3.50 (2H, s), 3.71 (3H, s), 3.99 (2H, d, J=6.1 Hz), 4.17 (2H, s), 5.78 (1H, d, J=2.8 Hz), 5.89 (1H, dd, J=7.5, 2.8 Hz), 6.23 (1H, t, J=6.5 Hz), 6.49 (2H, s), 6.75 (1H, d, J=7.6 Hz), 6.80-6.88 (2H, m), 6.97 (1H, d, J=6.0 Hz), 7.21 (1H, t, J=8.0 Hz), 7.32 (1H, d, J=8.3 Hz), 7.37-7.45 (2H, m), 7.53 (1H, d, J=7.6 Hz), 7.71 (1H, d, J=6.1 Hz), 8.35 (1H, s)

### Example Number 13009

### Pyridin-3-ylmethyl4-(((1-aminoisoquinolin-5-yl)amino)methyl)-1-(((1-methyl-2-oxo-1,2-dihydropyridin-4-yl)oxy)methyl)-2-azabicyclo[2.1.1]hexane-2-carboxylate

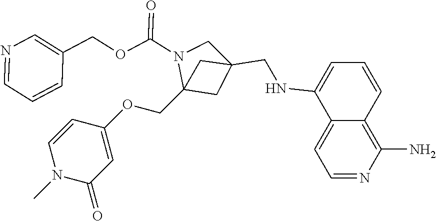 [SMILES: Cn1ccc(OCC23CC(CNc4cccc5c(N)nccc45)(CN2C(=O)OCc2cccnc2)C3)cc1=O]

### Pyridin-3-ylmethyl4-(((1-aminoisoquinolin-5-yl)amino)methyl)-1-(((1-methyl-2-oxo-1,2-dihydropyridin-4-yl)oxy)methyl)-2-azabicyclo[2.1.1]hexane-2-carboxylate (Example 13009)

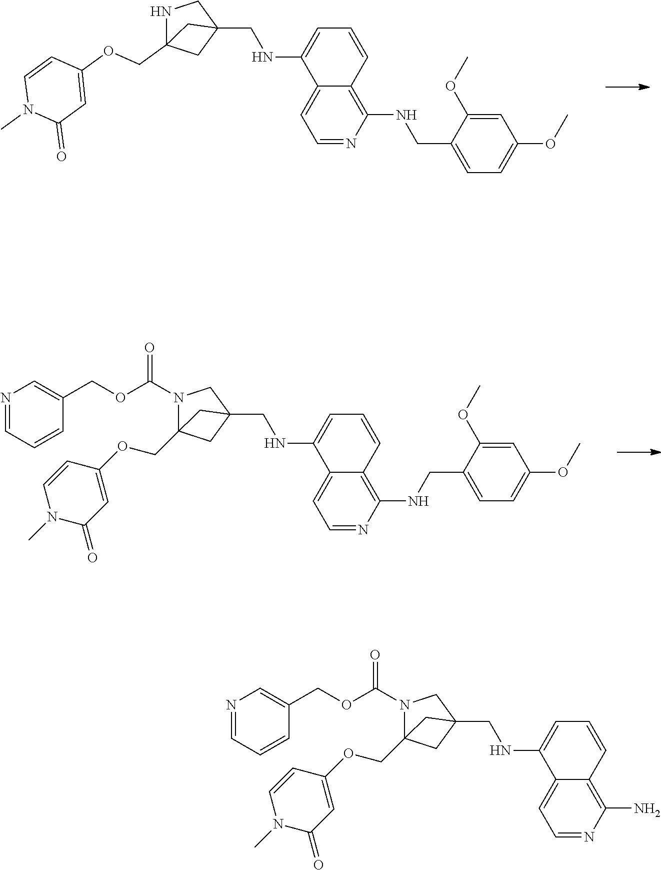

CDI (65 mg, 0.381 mmol) was added to a mixture of 3-pyridylmethanol (45.3 mg, 0.407 mmol) in MeCN (1 mL). The mixture was stirred at rt for 1 h and a solution of 4-((4-(((1-((2,4-dimethoxybenzyl)amino)isoquinolin-5-yl)amino)methyl)-2-azabicyclo[2.1.1]hexan-1-yl)methoxy)-1-methylpyridin-2(1H)-one (81.8 mg, 0.151 mmol) and DBU (10 μL, 0.0775 mmol) in MeCN (1 mL) was added at rt. The mixture was stirred at rt for 18 h and concentrated. The intermediate 3-pyridylmethyl 4-[[[1-[(2,4-dimethoxyphenyl)methylamino]-5-isoquinolyl]amino]methyl]-1-[(1-methyl-2-oxo-4-pyridyl)oxymethyl]-2-azabicyclo[2.1.1]hexane-2-carboxylate was deprotected according to General

Method 8 in DCM (750 μL) and TFA (750 μL, 10.0 mmol) at rt for 6 h. 2M NaOH(aq) (10 mL) was added and the mixture was stirred for 10 min. The aq phase was extracted with a mixture of DCM and MeOH (9:1 v/v, 3×20 mL). The combined organic phases were dried (Na₂SO₄), filtered and concentrated. The residue was diluted with a mixture of MeOH and MeCN (1:1 v/v, 2 mL) and loaded onto a PL-HCO₃ MP SPE cartridge (250 mg). The cartridge was washed with a mixture of MeOH and MeCN (1:1 v/v, 3×2 mL) and the filtrate was concentrated. The residue was purified by flash chromatography (Silica-C18, 25-38% MeCN in water [10 mM NH₄HCO₃]) and lyophilized to provide the product (50.2 mg, 63% yield over two steps).

[M+H]⁺=527.3.

¹H NMR (DMSO-d6, 400 MHz) δ 1.53-1.61 (2H, m), 1.83-1.90 (2H, m), 3.26 (3H, s), 3.40 (2H, s), 3.47 (2H, d, J=5.8 Hz), 4.42 (2H, s), 5.00 (2H, s), 5.60 (1H, s), 5.80 (1H, d, J=7.5 Hz), 5.94 (1H, t, J=6.0 Hz), 6.43 (2H, s), 6.63 (1H, d, J=7.6 Hz), 7.11 (1H, dd, J=6.4, 0.8 Hz), 7.15 (1H, t, J=8.0 Hz), 7.24-7.34 (2H, m), 7.45 (1H, d, J=7.6 Hz), 7.64-7.72 (2H, m), 8.45 (1H, d, J=4.8 Hz), 8.49 (1H, s).

### Examples

TABLE 14
Compound Names

| Example No | Name |
|---|---|
| 1001 | N5-((3-((imidazo[1,2-a]pyridin-7-yloxy)methyl)bicyclo[1.1.1]pentan-1- |
| | yl)methyl)isoquinoline-1,5-diamine |
| 1002 | N5-((3-(((5,6,7,8-tetrahydroimidazo[1,2-a]pyridin-7- |
| | yl)oxy)methyl)bicyclo[1.1.1]pentan-1-yl)methyl)isoquinoline-1,5-diamine |
| 1002.1 | (S*)-N5-((3-(((5,6,7,8-Tetrahydroimidazo[1,2-a]pyridin-7- |
| | yl)oxy)methyl)bicyclo[1.1.1]pentan-1-yl)methyl)isoquinoline-1,5-diamine |
| 1002.2 | (R*)-N5-((3-(((5,6,7,8-tetrahydroimidazo[1,2-a]pyridin-7- |
| | yl)oxy)methyl)bicyclo[1.1.1]pentan-1-yl)methyl)isoquinoline-1,5-diamine |
| 1003 | N5-((3-(((1-methylpiperidin-4-yl)oxy)methyl)bicyclo[1.1.1]pentan-1- |
| | yl)methyl)isoquinoline-1,5-diamine |
| 1004 | N5-((3-((5,6-dihydroimidazo[1,2-a]pyrazin-7(8H)-yl)methyl)bicyclo[1.1.1]pentan- |
| | 1-yl)methyl)isoquinoline-1,5-diamine |
| 1005 | N5-((3-((5-isopropyl-2,5-diazabicyclo[2.2.1]heptan-2- |
| | yl)methyl)bicyclo[1.1.1]pentan-1-yl)methyl)isoquinoline-1,5-diamine |
| 1005.1 | N5-((3-(((15,4S)-5-isopropyl-2,5-diazabicyclo[2.2.1]heptan-2- |
| | yl)methyl)bicyclo[1.1.1]pentan-1-yl)methyl)isoquinoline-1,5-diamine |
| 1005.2 | N5-((3-(((1R,4R)-5-isopropyl-2,5-diazabicyclo[2.2.1]heptan-2- |
| | yl)methyl)bicyclo[1.1.1]pentan-1-yl)methyl)isoquinoline-1,5-diamine |
| 1006 | N5-((3-(((2-methylimidazo[1,2-a]pyridin-7-yl)oxy)methyl)bicyclo[1.1.1]pentan-1- |
| | yl)methyl)isoquinoline-1,5-diamine |
| 1007 | N5-((3-(((8-methylimidazo[1,2-a]pyridin-7-yl)oxy)methyl)bicyclo[1.1.1]pentan-1- |
| | yl)methyl)isoquinoline-1,5-diamine |
| 1008 | 4-chloro-N6-((3-((imidazo[1,2-a]pyridin-7-yloxy)methyl)bicyclo[1.1.1]pentan-1- |
| | yl)methyl)isoquinoline-1,6-diamine |
| 1009 | N-((3-((imidazo[1,2-a]pyridin-7-yloxy)methyl)bicyclo[1.1.1]pentan-1-yl)methyl)- |
| | 1H-pyrrolo[2,3-b]pyridin-4-amine |
| 1010 | 3-chloro-N-((3-((imidazo[1,2-a]pyridin-7-yloxy)methyl)bicyclo[1.1.1]pentan-1- |
| | yl)methyl)-1H-pyrrolo[2,3-b]pyridin-5-amine |
| 1011 | N-((3-((imidazo[1,2-a]pyridin-7-yloxy)methyl)bicyclo[1.1.1]pentan-1- |
| | yl)methyl)isoquinolin-6-amine |
| 1012 | N-((3-((imidazo[1,2-a]pyridin-7-yloxy)methyl)bicyclo[1.1.1]pentan-1-yl)methyl)-2- |
| | methyl-1H-pyrrolo[2,3-b]pyridin-4-amine |
| 1013 | 5-[[3-(imidazo[1,2-a]pyridin-7-yloxymethyl)-1- |
| | bicyclo[1.1.1]pentanyl]methoxy]isoquinolin-1-amine |
| 1014 | 5-[2-[3-imidazo[1,2-a]pyridin-7-yloxymethyl)-1- |
| | bicyclo[1.1.1]pentanyl]ethyl]isoquinolin-1-amine |
| 1015 | [3-[(1-aminoisoquinolin-5-yl)oxymethyl]-1-bicyclo[1.1.1]pentanyl]-(6,8-dihydro- |
| | 5H-imidazo [1,2-a]pyrazin-7-yl)methanone |
| 1016 | [3-[[(1-aminoisoquinolin-5-yl)amino]methyl]-1-bicyclo[1.1.1]pentanyl]-(6,8- |
| | dihydro-5H-imidazo[1,2-a]pyrazin-7-yl)methanone |
| 1017 | 5-N-[[3-(imidazo[1,2-a]pyridin-6-yloxymethyl)-1- |
| | bicyclo[1.1.1]pentanyl]methyl]isoquinoline-1,5-diamine |
| 1018 | 5-N-[[3-(imidazo[1,2-a]pyridin-8-yloxymethyl)-1- |
| | bicyclo[1.1.1]pentanyl]methyllisoquinoline-1,5-diamine |
| 1019 | 4-[[3-[[(1-aminoisoquinolin-5-yl)amino]methyl]-1- |
| | bicyclo[1.1.1]pentanyl]methoxy]-1-methylpyridin-2-one |
| 1101 | 5-N-[[3-(1H-pyrrolo[3,2-b]pyridin-6-yloxymethyl)-1- |
| | bicyclo[1.1.1]pentanyl]methyl]isoquinoline-1,5-diamine |
| 1104 | 7-((3-(((1-aminoisoquinolin-5-yl)amino)methyl)bicyclo[1.1.1]pentan-1- |
| | yl)methoxy)quinazolin-4(1H)-one |
| 1105 | N5-((3-(((1H-benzo[d]imidazol-6-yl)oxy)methyl)bicyclo[1.1.1]pentan-1- |
| | yl)methyl)isoquinoline-1,5-diamine |
| 1106 | N5-((3-(((5,6,7,8-tetrahydroimidazo[1,2-a]pyridin-6- |
| | yl)oxy)methyl)bicyclo[1.1.1]pentan-1-yl)methyl)isoquinoline-1,5-diamine |
| 1108 | N5-((3-(((3-(difluoromethyl)-[1,2,4]triazolo[4,3-b]pyridazin-6- |
| | yl)oxy)methyl)bicyclo[1.1.1]pentan-1-yl)methyl)isoquinoline-1,5-diamine |
| 1109 | 6-((3-(((1-aminoisoquinolin-5-yl)amino)methyl)bicyclo[1.1.1]pentan-1- |
| | yl)methoxy)isoindolin-1-one |
| 1110 | N5-((3-(([1,2,4]triazolo[1,5-a]pyridin-7-yloxy)methyl)bicyclo[1.1.1]pentan-1- |
| | yl)methyl)isoquinoline-1,5-diamine |
| 1112 | 7-((3-(((1-aminoisoquinolin-5-yl)amino)methyl)bicyclo[1.1.1]pentan-1-yl)methyl)- |
| | 7,8-dihydroimidazo[1,2-a]pyrazin-6(5H)-one |
| 1113 | 6-((3-(((1-aminoisoquinolin-5-yl)amino)methyl)bicyclo[1.1.1]pentan-1- |
| | yl)methoxy)indolin-2-one |
| 1114 | N5-((3-(((octahydroindolizin-7-yl)oxy)methyl)bicyclo[1.1.1]pentan-1- |
| | yl)methyl)isoquinoline-1,5-diamine |
| 1115 | N5-((3-(((5-chloroimidazo[1,2-a]pyridin-7-yl)oxy)methyl)bicyclo[1.1.1]pentan-1- |
| | yl)methyl)isoquinoline-1,5-diamine |
| 1116 | N5-((3-(((5-fluoroimidazo[1,2-a]pyridin-7-yl)oxy)methyl)bicyclo[1.1.1]pentan-1- |
| | yl)methyl)isoquinoline-1,5-diamine |
| 1117 | N5-((3-(((5-methylimidazo[1,2-a]pyridin-7-yl)oxy)methyl)bicyclo[1.1.1]pentan-1- |
| | yl)methyl)isoquinoline-1,5-diamine |
| 1118 | N5-((3-(((6,7-dihydro-5H-pyrrolo[1,2-a]imidazol-6- |
| | yl)oxy)methyl)bicyclo[1.1.1]pentan-1-yl)methyl)isoquinoline-1,5-diamine |
| 1119 | N5-((3-(imidazo[1,2-a]pyridin-7-ylmethoxy)bicyclo[1.1.1]pentan-1- |
| | yl)methyl)isoquinoline-1,5-diamine |
| 1120 | 4-((3-((1-aminoisoquinolin-5-yl)amino)bicyclo[1.1.1]pentan-1-yl)methoxy)-1- |
| | methylpyridin-2(1H)-one |
| 1121 | N5-(3-((imidazo[1,2-a]pyridin-7-yloxy)methyl)bicyclo[1.1.1]pentan-1- |
| | yl)isoquinoline-1,5-diamine |
| 1122 | N5-(3-(((5,6,7,8-tetrahydroimidazo[1,2-a]pyridin-7- |
| | yl)oxy)methyl)bicyclo[1.1.1]pentan-1-yl)isoquinoline-1,5-diamine |
| 1123 | N5-(3-(imidazo[1,2-a]pyridin-7-ylmethoxy)bicyclo[1.1.1]pentan-1-yl)isoquinoline- |
| | 1,5-diamine |
| 1124 | N5-(3-((5,6,7,8-tetrahydroimidazo[1,2-a]pyridin-7- |
| | yl)methoxy)bicyclo[1.1.1]pentan-1-yl)isoquinoline-1,5-diamine |
| 1125 | N5-((3-(((1H-indazol-6-yl)oxy)methyl)bicyclo[1.1.1]pentan-1- |
| | yl)methyl)isoquinoline-1,5-diamine |
| 1126 | N5-((3-(imidazo[1,2-a]pyridin-1(8aH)-ylmethyl)bicyclo[1.1.1]pentan-1- |
| | yl)methyl)isoquinoline-1,5-diamine |
| 1127 | 1-((3-(((1-aminoisoquinolin-5-yl)amino)methyl)bicyclo[1.1.1]pentan-1- |
| | yl)methyl)imidazo[1,2-a]pyridin-7(1H)-one |
| 1129 | 3-chloro-N-((3-((imidazo[1,2-a]pyridin-7-yloxy)methyl)bicyclo[1.1.1]pentan-1- |
| | yl)methyl)-1H-pyrrolo[2,3-b]pyridin-5-amine |
| 1130 | 2-chloro-N-((3-(((5,6,7,8-tetrahydroimidazo[1,2-a]pyridin-7- |
| | yl)oxy)methyl)bicyclo[1.1.1]pentan-1-yl)methyl)-1H-pyrrolo[2,3-b]pyridin-4- |
| | amine |
| 1131 | N5-(3-((imidazo[1,2-a]pyridin-6-yloxy)methyl)bicyclo[1.1.1]pentan-1- |
| | yl)isoquinoline-1,5-diamine |
| 1132 | N-(3-(((1-aminoisoquinolin-5-yl)amino)methyl)bicyclo[1.1.1]pentan-1-yl)-3- |
| | (difluoromethyl)-[1,2,4]triazolo[4,3-a]pyridine-6-carboxamide |
| 1133 | 5-((3-(((1-aminoisoquinolin-5-yl)amino)methyl)bicyclo[1.1.1]pentan-1- |
| | yl)methoxy)isoindolin-1-one |
| 1134 | 4-((3-(((1-aminoisoquinolin-5-yl)amino)methyl)bicyclo[1.1.1]pentan-1- |
| | yl)methoxy)-1,6-dimethylpyridin-2(1H)-one |
| 1135 | N5-((3-(([1,2,4]triazolo[4,3-a]pyridin-7-yloxy)methyl)bicyclo[1.1.1]pentan-1- |
| | yl)methyl)isoquinoline-1,5-diamine |
| 2020 | 5-N-[[1-(imidazo[1,2-a]pyridin-7-yloxymethyl)-2-oxabicyclo[2.1.1]hexan-4- |
| | yl]methyl]isoquinoline-1,5-diamine |
| 2021 | N-((1-((imidazo[1,2-a]pyridin-7-yloxy)methyl)-2-oxabicyclo[2.1.1]hexan-4- |
| | yl)methyl)-2-methyl-1H-pyrrolo[2,3-b]pyridin-4-amine |
| 2022 | 5-N-[[1-(imidazo[1,2-a]pyridin-6-yloxymethyl)-2-oxabicyclo[2.1.1]hexan-4- |
| | yl]methyl]isoquinoline-1,5-diamine |
| 2201 | 5-((1-((imidazo[1,2-a]pyridin-7-yloxy)methyl)-2-azabicyclo[2.1.1]hexan-4- |
| | yl)methoxy)isoquinolin-1-amine |
| 2202 | 1-(4-(((1-aminoisoquinolin-5-yl)oxy)methyl)-1-((imidazo[1,2-a]pyridin-7- |
| | yloxy)methyl)-2-azabicyclo[2.1.1]hexan-2-yl)ethan-1-one |
| 2203 | 5-((4-((imidazo[1,2-a]pyridin-7-yloxy)methyl)bicyclo[2.1.1]hexan-1- |
| | yl)methoxy)isoquinolin-1-amine |
| 2204 | N5-((1-(((3-(difluoromethyl)-[1,2,4]triazolo[4,3-b]pyridazin-6-yl)oxy)methyl)-2- |
| | oxabicyclo[2.1.1]hexan-4-yl)methyl)isoquinoline-1,5-diamine |
| 2205 | 4-((4-(((1-aminoisoquinolin-5-yl)amino)methyl)-2-oxabicyclo[2.1.1]hexan-1- |
| | yl)methoxy)-1-methylpyridin-2(1H)-one |
| 2206 | N5-((1-(((3-(trifluoromethyl)-[1,2,4]triazolo[4,3-a]pyridin-7-yl)oxy)methyl)-2- |
| | oxabicyclo[2.1.1]hexan-4-yl)methyl)isoquinoline-1,5-diamine |
| 2207 | N5-((1-(([1,2,4]triazolo[1,5-a]pyridin-7-yloxy)methyl)-2-oxabicyclo[2.1.1]hexan-4- |
| | yl)methyl)isoquinoline-1,5-diamine |
| 2208 | 2-((4-(((1-aminoisoquinolin-5-yl)amino)methyl)-2-oxabicyclo[2.1.1]hexan-1- |
| | yl)methoxy)pyrimidine-5-carbonitrile |
| 2210 | N5-((1-(([1,2,4]triazolo[1,5-a]pyridin-6-yloxy)methyl)-2-oxabicyclo[2.1.1]hexan-4- |
| | yl)methyl)isoquinoline-1,5-diamine |
| 2211 | 6-((4-(((1-aminoisoquinolin-5-yl)amino)methyl)-2-oxabicyclo[2.1.1]hexan-1- |
| | yl)methoxy)-2-methylisoindolin-1-one |
| 2212 | N5-((1-(((3-methylimidazo[1,2-a]pyridin-6-yl)oxy)methyl)-2- |
| | oxabicyclo[2.1.1]hexan-4-yl)methyl)isoquinoline-1,5-diamine |
| 3023 | N5-((1-((imidazo[1,2-a]pyridin-7-yloxy)methyl)-3,3-dimethyl-2- |
| | oxabicyclo[2.1.1]hexan-4-yl)methyl)isoquinoline-1,5-diamine |
| 3024 | 5-((1-((imidazo[1,2-a]pyridin-7-yloxy)methyl)-3,3-dimethyl-2- |
| | oxabicyclo[2.1.1]hexan-4-yl)methoxy)isoquinolin-1-amine |
| 4401 | N5-((trans-3-((imidazo[1,2-a]pyridin-7- |
| | yloxy)methyl)cyclobutyl)methyl)isoquinoline-1,5-diamine |
| 4402 | N5-((trans-3-(imidazo[1,2-a]pyridin-7-ylmethoxy)cyclobutyl)methyl)isoquinoline- |
| | 1,5-diamine |
| 4403 | 4-((trans-3-(((1-aminoisoquinolin-5-yl)amino)methyl)cyclobutoxy)methyl)-1- |
| | methylpyridin-2(1H)-one |
| 5003 | N5-((cis-3-((imidazo[1,2-a]pyridin-7-yloxy)methyl)cyclobutyl)methyl)isoquinoline- |
| | 1,5-diamine |
| 6601 | 5-N-[[4-(imidazo[1,2-a]pyridin-7-yloxymethyl)-2-oxabicyclo[2.1.1]hexan-1- |
| | yl]methyl]isoquinoline-1,5-diamine |
| 6602 | N5-((4-((imidazo[1,2-a]pyridin-6-yloxy)methyl)-2-oxabicyclo[2.1.1]hexan-1- |
| | yl)methyl)isoquinoline-1,5-diamine |
| 6603 | 5-((4-((imidazo[1,2-a]pyridin-7-yloxy)methyl)-2-azabicyclo[2.1.1]hexan-1- |
| | yl)methoxy)isoquinolin-1-amine |
| 6604 | 1-(1-(((1-aminoisoquinolin-5-yl)oxy)methyl)-4-((imidazo[1,2-a]pyridin-7- |
| | yloxy)methyl)-2-azabicyclo[2.1.1]hexan-2-yl)ethan-1-one |
| 6605 | N-((4-((imidazo[1,2-a]pyridin-7-yloxy)methyl)-2-oxabicyclo[2.1.1]hexan-1- |
| | yl)methyl)-2-methyl-1H-pyrrolo[2,3-b]pyridin-4-amine |
| 6606 | 2-chloro-N-((4-((imidazo[1,2-a]pyridin-7-yloxy)methyl)-2-oxabicyclo[2.1.1]hexan- |
| | 1-yl)methyl)-1H-pyrrolo[2,3-b]pyridin-4-amine |
| 6607 | N-((4-((imidazo[1,2-a]pyridin-7-yloxy)methyl)-2-oxabicyclo[2.1.1]hexan-1- |
| | yl)methyl)-2-methyl-1H-pyrrolo[2,3-c]pyridin-4-amine |
| 6608 | N5-((4-(((3-(difluoromethyl)-[1,2,4]triazolo[4,3-b]pyridazin-6-yl)oxy)methyl)-2- |
| | oxabicyclo[2.1.1]hexan-1-yl)methyl)isoquinoline-1,5-diamine |
| 6609 | 4-((1-(((1-aminoisoquinolin-5-yl)amino)methyl)-2-oxabicyclo[2.1.1]hexan-4- |
| | yl)methoxy)-1-methylpyridin-2(1H)-one |
| 6610 | N5-((4-(((5,6,7,8-tetrahydroimidazo[1,2-a]pyridin-6-yl)oxy)methyl)-2- |
| | oxabicyclo[2.1.1]hexan-1-yl)methyl)isoquinoline-1,5-diamine |
| 6611 | 2-chloro-N-((4-((imidazo[1,2-a]pyridin-6-yloxy)methyl)-2-oxabicyclo[2.1.1]hexan- |
| | 1-yl)methyl)-1H-pyrrolo[2,3-b]pyridin-4-amine |
| 6612 | N5-((4-((imidazo[1,2-b]pyridazin-6-yloxy)methyl)-2-oxabicyclo[2.1.1]hexan-1- |
| | yl)methyl)isoquinoline-1,5-diamine |
| 6613 | N5-((4-(((2-(difluoromethyl)imidazo[1,2-a]pyridin-7-yl)oxy)methyl)-2- |
| | oxabicyclo[2.1.1]hexan-1-yl)methyl)isoquinoline-1,5-diamine |
| 6614 | N5-((4-((imidazo[1,2-a]pyrimidin-7-yloxy)methyl)-2-oxabicyclo[2.1.1]hexan-1- |
| | yl)methyl)isoquinoline-1,5-diamine |
| 6615 | N5-((4-(((2-(trifluoromethyl)imidazo[1,2-b]pyridazin-6-yl)oxy)methyl)-2- |
| | oxabicyclo[2.1.1]hexan-1-yl)methyl)isoquinoline-1,5-diamine |
| 6616 | N5-((4-((imidazo[1,2-a]pyrazin-6-yloxy)methyl)-2-oxabicyclo[2.1.1]hexan-1- |
| | yl)methyl)isoquinoline-1,5-diamine |
| 6617 | N5-((4-(((8-methylimidazo[1,2-a]pyridin-6-yl)oxy)methyl)-2- |
| | oxabicyclo[2.1.1]hexan-1-yl)methyl)isoquinoline-1,5-diamine |
| 6618 | 5-((1-(((1-aminoisoquinolin-5-yl)amino)methyl)-2-oxabicyclo[2.1.1]hexan-4- |
| | yl)methoxy)isoindolin-1-one |
| 6619 | 5-((4-((imidazo[1,2-a]pyridin-6-ylamino)methyl)-2-oxabicyclo[2.1.1]hexan-1- |
| | yl)methoxy)isoquinolin-1-amine |
| 6620 | N5-((4-(((3-(difluoromethyl)-[1,2,4]triazolo[4,3-a]pyridin-6-yl)oxy)methyl)-2- |
| | oxabicyclo[2.1.1]hexan-1-yl)methyl)isoquinoline-1,5-diamine |
| 6621 | 5-((1-(((1-aminoisoquinolin-5-yl)amino)methyl)-2-oxabicyclo[2.1.1]hexan-4- |
| | yl)methoxy)-2-methylisoindolin-1-one |
| 6622 | N5-((4-(((7-methylimidazo[1,2-a]pyridin-6-yl)oxy)methyl)-2- |
| | oxabicyclo[2.1.1]hexan-1-yl)methyl)isoquinoline-1,5-diamine |
| 6623 | N5-((4-((imidazo[1,2-c]pyrimidin-7-yloxy)methyl)-2-oxabicyclo[2.1.1]hexan-1- |
| | yl)methyl)isoquinoline-1,5-diamine |
| 6624 | N5-((4-(((3-methylimidazo[1,2-a]pyridin-6-yl)oxy)methyl)-2- |
| | oxabicyclo[2.1.1]hexan-1-yl)methyl)isoquinoline-1,5-diamine |
| 6625 | N5-((4-(((3-fluoroimidazo[1,2-a]pyridin-6-yl)oxy)methyl)-2- |
| | oxabicyclo[2.1.1]hexan-1-yl)methyl)isoquinoline-1,5-diamine |
| 6626 | N5-((4-(((5-methylimidazo[1,2-a]pyridin-6-yl)oxy)methyl)-2- |
| | oxabicyclo[2.1.1]hexan-1-yl)methyl)isoquinoline-1,5-diamine |
| 6627 | N5-((4-(((3-(trifluoromethyl)imidazo[1,2-a]pyridin-6-yl)oxy)methyl)-2- |
| | oxabicyclo[2.1.1]hexan-1-yl)methyl)isoquinoline-1,5-diamine |
| 6628 | 6-((1-(((1-aminoisoquinolin-5-yl)amino)methyl)-2-oxabicyclo[2.1.1]hexan-4- |
| | yl)methoxy)-2-methylisoindolin-1-one |
| 6629 | N5-((4-(([1,2,4]triazolo[4,3-a]pyridin-7-yloxy)methyl)-2-oxabicyclo[2.1.1]hexan-1- |
| | yl)methyl)isoquinoline-1,5-diamine |
| 6630 | N5-((4-(((8-(trifluoromethyl)imidazo[1,2-a]pyridin-6-yl)oxy)methyl)-2- |
| | oxabicyclo[2.1.1]hexan-1-yl)methyl)isoquinoline-1,5-diamine |
| 6631 | N5-((4-(((8-methoxyimidazo[1,2-a]pyridin-6-yl)oxy)methyl)-2- |
| | oxabicyclo[2.1.1]hexan-1-yl)methyl)isoquinoline-1,5-diamine |
| 6633 | N5-((4-(((2-methylimidazo[1,2-a]pyridin-6-yl)oxy)methyl)-2- |
| | oxabicyclo[2.1.1]hexan-1-yl)methyl)isoquinoline-1,5-diamine |
| 6634 | N5-((4-(([1,2,4]triazolo[4,3-a]pyridin-6-yloxy)methyl)-2-oxabicyclo[2.1.1]hexan-1- |
| | yl)methyl)isoquinoline-1,5-diamine |
| 6635 | 2-((1-(((1-aminoisoquinolin-5-yl)amino)methyl)-2-oxabicyclo[2.1.1]hexan-4- |
| | yl)methyl)-5-hydroxyisoindoline-1,3-dione |
| 6636 | 5-((1-(((1-aminoisoquinolin-5-yl)amino)methyl)-2-oxabicyclo[2.1.1]hexan-4- |
| | yl)methoxy)isoindoline-1,3-dione |
| 6637 | 6-((1-(((1-aminoisoquinolin-5-yl)amino)methyl)-2-oxabicyclo[2.1.1]hexan-4- |
| | yl)methoxy)-3,4-dihydroisoquinolin-1(2H)-one |
| 6638 | N5-((4-(([1,2,4]triazolo[4,3-b]pyridazin-6-yloxy)methyl)-2-oxabicyclo[2.1.1]hexan- |
| | 1-yl)methyl)isoquinoline-1,5-diamine |
| 6639 | N5-((4-(((3-cyclopropylimidazo[1,2-a]pyridin-6-yl)oxy)methyl)-2- |
| | oxabicyclo[2.1.1]hexan-1-yl)methyl)isoquinoline-1,5-diamine |
| 6640 | 6-((1-(((1-aminoisoquinolin-5-yl)amino)methyl)-2-oxabicyclo[2.1.1]hexan-4- |
| | yl)methoxy)-2-methyl-3,4-dihydroisoquinolin-1(2H)-one |
| 6641 | N5-((4-(((8-isopropylimidazo[1,2-a]pyridin-6-yl)oxy)methyl)-2- |
| | oxabicyclo[2.1.1]hexan-1-yl)methyl)isoquinoline-1,5-diamine |
| 6642 | 6-((1-(((1-aminoisoquinolin-5-yl)amino)methyl)-2-oxabicyclo[2.1.1]hexan-4- |
| | yl)methoxy)isoindolin-1-one |
| 6643 | 6-((1-(((1-aminoisoquinolin-5-yl)amino)methyl)-2-oxabicyclo[2.1.1]hexan-4- |
| | yl)methoxy)-2-methylisoquinolin-1(2H)-one |
| 6644 | N5-((4-(((3-(difluoromethyl)imidazo[1,2-a]pyridin-6-yl)oxy)methyl)-2- |
| | oxabicyclo[2.1.1]hexan-1-yl)methyl)isoquinoline-1,5-diamine |
| 6645 | N5-((4-((1H-pyrazol-1-yl)methyl)-2-oxabicyclo[2.1.1]hexan-1- |
| | yl)methyl)isoquinoline-1,5-diamine |
| 6646 | N5-((4-((1H-imidazol-1-yl)methyl)-2-oxabicyclo[2.1.1]hexan-1- |
| | yl)methyl)isoquinoline-1,5-diamine |
| 6647 | N5-((4-((2H-1,2,3-triazol-2-yl)methyl)-2-oxabicyclo[2.1.1]hexan-1- |
| | yl)methyl)isoquinoline-1,5-diamine |
| 6648 | N5-((4-((1H-1,2,3-triazol-1-yl)methyl)-2-oxabicyclo[2.1.1]hexan-1- |
| | yl)methyl)isoquinoline-1,5-diamine |
| 6649 | 1-((1-(((1-aminoisoquinolin-5-yl)amino)methyl)-2-oxabicyclo[2.1.1]hexan-4- |
| | yl)methyl)pyrrolidin-2-one |
| 6650 | 5-((1-(((1-aminoisoquinolin-5-yl)amino)methyl)-2-oxabicyclo[2.1.1]hexan-4- |
| | yl)methoxy)-3,3-dimethylisoindolin-1-one |
| 6651 | 5-((1-(((2-chloro-1H-pyrrolo[2,3-b]pyridin-4-yl)amino)methyl)-2- |
| | oxabicyclo[2.1.1]hexan-4-yl)methoxy)isoindolin-1-one |
| 6653 | 1-(3-((1-(((1-aminoisoquinolin-5-yl)amino)methyl)-2-oxabicyclo[2.1.1]hexan-4- |
| | yl)methoxy)phenyl)pyrrolidin-2-one |
| 6654 | 1-(2-((1-(((1-aminoisoquinolin-5-yl)amino)methyl)-2-oxabicyclo[2.1.1]hexan-4- |
| | yl)methoxy)phenyl)pyrrolidin-2-one |
| 6656 | 1,3-dimethyl-N-((4-(((3-methylimidazo[1,2-a]pyridin-6-yl)oxy)methyl)-2- |
| | oxabicyclo[2.1.1]hexan-1-yl)methyl)-1H-indol-6-amine |
| 6658 | 2-(((4-(((3-methylimidazo[1,2-a]pyridin-6-yl)oxy)methyl)-2- |
| | oxabicyclo[2.1.1]hexan-1-yl)methyl)amino)benzonitrile |
| 6659 | N-((4-(((3-methylimidazo[1,2-a]pyridin-6-yl)oxy)methyl)-2- |
| | oxabicyclo[2.1.1]hexan-1-yl)methyl)-2-(2,2,2-trifluoroethyl)aniline |
| 6660 | N4-((4-(((3-methylimidazo[1,2-a]pyridin-6-yl)oxy)methyl)-2- |
| | oxabicyclo[2.1.1]hexan-1-yl)methyl)pyridine-2,4-diamine |
| 6661 | N-((4-(((3-methylimidazo[1,2-a]pyridin-6-yl)oxy)methyl)-2- |
| | oxabicyclo[2.1.1]hexan-1-yl)methyl)pyridin-3-amine |
| 6663 | 4-methoxy-N-((4-(((3-methylimidazo[1,2-a]pyridin-6-yl)oxy)methyl)-2- |
| | oxabicyclo[2.1.1]hexan-1-yl)methyl)pyridin-2-amine |
| 7701 | N5-((4-((imidazo[1,2-a]pyridin-7-yloxy)methyl)-3,3-dimethyl-2- |
| | oxabicyclo[2.1.1]hexan-1-yl)methyl)isoquinoline-1,5-diamine |
| 7702 | 5-((4-((imidazo[1,2-a]pyridin-7-yloxy)methyl)-3,3-dimethyl-2- |
| | oxabicyclo[2.1.1]hexan-1-yl)methoxy)isoquinolin-1-amine |
| 7703 | N5-((4-((imidazo[1,2-a]pyridin-6-yloxy)methyl)-3,3-dimethyl-2- |
| | oxabicyclo[2.1.1]hexan-1-yl)methyl)isoquinoline-1,5-diamine |
| 7704 | 5-((4-((imidazo[1,2-a]pyridin-6-yloxy)methyl)-3,3-dimethyl-2- |
| | oxabicyclo[2.1.1]hexan-1-yl)methoxy)isoquinolin-1-amine |
| 8801 | 5-(6-(imidazo[1,2-a]pyridin-7-ylmethoxy)-2-azaspiro[3.3]heptan-2-yl)isoquinolin- |
| | 1-amine |
| 8802 | 6-(6-(imidazo[1,2-a]pyridin-7-ylmethoxy)-2-azaspiro[3.3]heptan-2-yl)isoquinoline |
| 8803 | 5-(2-(imidazo[1,2-a]pyridin-7-ylmethoxy)-6-azaspiro[3.4]octan-6-yl)isoquinolin-1- |
| | amine |
| 8804 | 5-(cis*-2-(imidazo[1,2-a]pyridin-7-ylmethoxy)-6-azaspiro[3.4]octan-6- |
| | yl)isoquinolin-1-amine |
| 8805 | 5-(trans*-2-(imidazo[1,2-a]pyridin-7-ylmethoxy)-6-azaspiro[3.4]octan-6- |
| | yl)isoquinolin-1-amine |
| 8806 | 5-(trans*-2-((imidazo[1,2-a]pyridin-7-yloxy)methyl)-6-azaspiro[3.4]octan-6- |
| | yl)isoquinolin-1-amine |
| 8807 | 5-(cis*-2-((imidazo[1,2-a]pyridin-7-yloxy)methyl)-6-azaspiro[3.4]octan-6- |
| | yl)isoquinolin-1-amine |
| 8808 | 5-(6-((imidazo[1,2-a]pyridin-6-yloxy)methyl)-2-azaspiro[3.3]heptan-2- |
| | yl)isoquinolin-1-amine |
| 9001 | 5-(5-(imidazo[1,2-a]pyridin-7-ylmethoxy)-2-azaspiro[3.3]heptan-2-yl)isoquinolin- |
| | 1-amine |
| 9002 | (R*)-5-(5-(imidazo[1,2-a]pyridin-7-ylmethoxy)-2-azaspiro[3.3]heptan-2- |
| | yl)isoquinolin-1-amine |
| 9003 | (S*)-5-(5-(imidazo[1,2-a]pyridin-7-ylmethoxy)-2-azaspiro[3.3]heptan-2- |
| | yl)isoquinolin-1-amine |
| 10901 | N5-((5-((imidazo[1,2-a]pyridin-7-yloxy)methyl)-2-oxabicyclo[3.1.1]heptan-1- |
| | yl)methyl)isoquinoline-1,5-diamine |
| 10902 | N5-((5-((imidazo[1,2-a]pyridin-7-yloxy)methyl)bicyclo[3.1.1]heptan-1- |
| | yl)methyl)isoquinoline-1,5-diamine |
| 10903 | N5-((5-(((5,6,7,8-tetrahydroimidazo[1,2-a]pyridin-7-yl)oxy)methyl)-2- |
| | oxabicyclo[3.1.1]heptan-1-yl)methyl)isoquinoline-1,5-diamine |
| 10904 | N5-((5-((imidazo[1,2-a]pyridin-6-yloxy)methyl)-2-oxabicyclo[3.1.1]heptan-1- |
| | yl)methyl)isoquinoline-1,5-diamine |
| 10906 | 4-((1-(((1-aminoisoquinolin-5-yl)amino)methyl)-2-oxabicyclo[3.1.1]heptan-5- |
| | yl)methoxy)-1-methylpyridin-2(1H)-one |
| 10907 | N-((5-((imidazo[1,2-a]pyridin-7-yloxy)methyl)-2-oxabicyclo[3.1.1]heptan-1- |
| | yl)methyl)-2-methyl-1H-pyrrolo[2,3-b]pyridin-4-amine |
| 10908 | N5-((5-(((5,6,7,8-tetrahydroimidazo[1,2-a]pyridin-6-yl)oxy)methyl)-2- |
| | oxabicyclo[3.1.1]heptan-1-yl)methyl)isoquinoline-1,5-diamine |
| 10909 | 2-chloro-N-((5-((imidazo[1,2-a]pyridin-6-yloxy)methyl)-2-oxabicyclo[3.1.1]heptan- |
| | 1-yl)methyl)-1H-pyrrolo[2,3-b]pyridin-4-amine |
| 10910 | N5-((5-(((3-(difluoromethyl)-[1,2,4]triazolo[4,3-b]pyridazin-6-yl)oxy)methyl)-2- |
| | oxabicyclo[3.1.1]heptan-1-yl)methyl)isoquinoline-1,5-diamine |
| 10911 | 3-((1-(((1-aminoisoquinolin-5-yl)amino)methyl)-2-oxabicyclo[3.1.1]heptan-5- |
| | yl)methoxy)-1-methylpyridin-2(1H)-one |
| 10912 | N5-((5-((pyrimidin-2-yloxy)methyl)-2-oxabicyclo[3.1.1]heptan-1- |
| | yl)methyl)isoquinoline-1,5-diamine |
| 10914 | N5-((5-((pyridin-2-yloxy)methyl)-2-oxabicyclo[3.1.1]heptan-1- |
| | yl)methyl)isoquinoline-1,5-diamine |
| 10915 | 1-((1-(((1-aminoisoquinolin-5-yl)amino)methyl)-2-oxabicyclo[3.1.1]heptan-5- |
| | yl)methyl)pyridin-2(1H)-one |
| 10918 | 4-chloro-N-((5-((imidazo[1,2-a]pyridin-7-yloxy)methyl)-2-oxabicyclo[3.1.1]heptan- |
| | 1-yl)methyl)aniline |
| 10919 | 3-chloro-N-((5-((imidazo[1,2-a]pyridin-7-yloxy)methyl)-2-oxabicyclo[3.1.1]heptan- |
| | 1-yl)methyl)aniline |
| 10920 | N5-((5-(((6-methylimidazo[1,2-a]pyridin-7-yl)oxy)methyl)-2- |
| | oxabicyclo[3.1.1]heptan-1-yl)methyl)isoquinoline-1,5-diamine |
| 10921 | 7-chloro-N-((5-((imidazo[1,2-a]pyridin-7-yloxy)methyl)-2-oxabicyclo[3.1.1]heptan- |
| | 1-yl)methyl)quinolin-4-amine |
| 10922 | 4-chloro-N6-((5-((imidazo[1,2-a]pyridin-7-yloxy)methyl)-2- |
| | oxabicyclo[3.1.1]heptan-1-yl)methyl)isoquinoline-1,6-diamine |
| 10923 | N5-((5-(((8-methylimidazo[1,2-a]pyridin-7-yl)oxy)methyl)-2- |
| | oxabicyclo[3.1.1]heptan-1-yl)methyl)isoquinoline-1,5-diamine |
| 10924 | N5-((5-(((8-chloroimidazo[1,2-a]pyridin-6-yl)oxy)methyl)-2- |
| | oxabicyclo[3.1.1]heptan-1-yl)methyl)isoquinoline-1,5-diamine |
| 10925 | N5-((5-(((5-methylimidazo[1,2-a]pyridin-7-yl)oxy)methyl)-2- |
| | oxabicyclo[3.1.1]heptan-1-yl)methyl)isoquinoline-1,5-diamine |
| 10926 | N5-((5-(([1,2,4]triazolo[4,3-a]pyridin-7-yloxy)methyl)-2-oxabicyclo[3.1.1]heptan- |
| | 1-yl)methyl)isoquinoline-1,5-diamine |
| 10928 | 4-((1-(((1-aminoisoquinolin-5-yl)amino)methyl)-2-oxabicyclo[3.1.1]heptan-5- |
| | yl)methoxy)-1,6-dimethylpyridin-2(1H)-one |
| 10929 | N5-((5-(([1,2,4]triazolo[4,3-a]pyridin-6-yloxy)methyl)-2-oxabicyclo[3.1.1]heptan- |
| | 1-yl)methyl)isoquinoline-1,5-diamine |
| 10930 | N5-((5-(((3-methylimidazo[1,2-a]pyridin-7-yl)oxy)methyl)-2- |
| | oxabicyclo[3.1.1]heptan-1-yl)methyl)isoquinoline-1,5-diamine |
| 10931 | 5-((1-(((1-aminoisoquinolin-5-yl)amino)methyl)-2-oxabicyclo[3.1.1]heptan-5- |
| | yl)methoxy)isoindolin-1-one |
| 10932 | N5-((5-(((3-(trifluoromethyl)imidazo[1,2-a]pyridin-7-yl)oxy)methyl)-2- |
| | oxabicyclo[3.1.1]heptan-1-yl)methyl)isoquinoline-1,5-diamine |
| 10933 | 4-((1-(((1-aminoisoquinolin-5-yl)amino)methyl)-2-oxabicyclo[3.1.1]heptan-5- |
| | yl)methoxy)-5-chloro-1-methylpyridin-2(1H)-one |
| 10934 | 4-((1-(((1-aminoisoquinolin-5-yl)amino)methyl)-2-oxabicyclo[3.1.1]heptan-5- |
| | yl)methoxy)-1-isopropylpyridin-2(1H)-one |
| 10936 | N5-((5-((isoxazol-3-yloxy)methyl)-2-oxabicyclo[3.1.1]heptan-1- |
| | yl)methyl)isoquinoline-1,5-diamine |
| 10937 | N5-((5-((pyrimidin-4-yloxy)methyl)-2-oxabicyclo[3.1.1]heptan-1- |
| | yl)methyl)isoquinoline-1,5-diamine |
| 10938 | 6-((1-(((1-aminoisoquinolin-5-yl)amino)methyl)-2-oxabicyclo[3.1.1]heptan-5- |
| | yl)methoxy)-3,4-dihydroisoquinolin-1(2H)-one |
| 10939 | 6-((1-(((1-aminoisoquinolin-5-yl)amino)methyl)-2-oxabicyclo[3.1.1]heptan-5- |
| | yl)methoxy)-2-methyl-3,4-dihydroisoquinolin-1(2H)-one |
| 10940 | 4-((1-(((1-aminoisoquinolin-5-yl)amino)methyl)-2-oxabicyclo[3.1.1]heptan-5- |
| | yl)methoxy)-1-methylpyrimidin-2(1H)-one |
| 10941 | 6-((1-(((1-aminoisoquinolin-5-yl)amino)methyl)-2-oxabicyclo[3.1.1]heptan-5- |
| | yl)methoxy)isoindolin-1-one |
| 10942 | N5-((5-((imidazo[1,2-a]pyridin-6-yloxy)methyl)bicyclo[3.1.1]heptan-1- |
| | yl)methyl)isoquinoline-1,5-diamine |
| 10943 | 5-((1-(((1-aminoisoquinolin-5-yl)amino)methyl)-2-oxabicyclo[3.1.1]heptan-5- |
| | yl)methoxy)-1-methylpyridin-2(1H)-one |
| 10944 | N5-((5-(((2-methoxypyridin-4-yl)oxy)methyl)-2-oxabicyclo[3.1.1]heptan-1- |
| | yl)methyl)isoquinoline-1,5-diamine |
| 10945 | 6-((1-(((1-aminoisoquinolin-5-yl)amino)methyl)-2-oxabicyclo[3.1.1]heptan-5- |
| | yl)methoxy)-1-methylpyridin-2(1H)-one |
| 10946 | N5-((5-(((1-methyl-1H-1,2,3-triazol-5-yl)oxy)methyl)-2-oxabicyclo[3.1.1]heptan-1- |
| | yl)methyl)isoquinoline-1,5-diamine |
| 10948 | N5-((5-(((6-isopropylpyrimidin-4-yl)oxy)methyl)-2-oxabicyclo[3.1.1]heptan-1- |
| | yl)methyl)isoquinoline-1,5-diamine |
| 10949 | 4-((1-(((1-aminoisoquinolin-5-yl)amino)methyl)-2-oxabicyclo[3.1.1]heptan-5- |
| | yl)methoxy)-6-isopropyl-1-methylpyridin-2(1H)-one |
| 10951 | 4-((1-(((1-aminoisoquinolin-5-yl)amino)methyl)-2-oxabicyclo[3.1.1]heptan-5- |
| | yl)methoxy)-1-methyl-6-(trifluoromethyl)pyridin-2(1H)-one |
| 10952 | 1-(((1-aminoisoquinolin-5-yl)amino)methyl)-N-(4-methoxybenzyl)-4-(((1-methyl- |
| | 2-oxo-1,2-dihydropyridin-4-yl)oxy)methyl)-2-azabicyclo[2.1.1]hexane-2- |
| | carboxamide |
| 10953 | N5-((5-(((2-methylpyridin-4-yl)oxy)methyl)-2-oxabicyclo[3.1.1]heptan-1- |
| | yl)methyl)isoquinoline-1,5-diamine |
| 10954 | N5-((5-(((4-methoxypyridin-3-yl)oxy)methyl)-2-oxabicyclo[3.1.1]heptan-1- |
| | yl)methyl)isoquinoline-1,5-diamine |
| 10955 | N5-((5-(((4-methylpyridin-2-yl)oxy)methyl)-2-oxabicyclo[3.1.1]heptan-1- |
| | yl)methyl)isoquinoline-1,5-diamine |
| 10956 | N5-((5-(((5-methylpyridin-2-yl)oxy)methyl)-2-oxabicyclo[3.1.1]heptan-1- |
| | yl)methyl)isoquinoline-1,5-diamine |
| 10957 | N5-((5-(((6-methylpyridin-2-yl)oxy)methyl)-2-oxabicyclo[3.1.1]heptan-1- |
| | yl)methyl)isoquinoline-1,5-diamine |
| 10958 | N-((5-((imidazo[1,2-a]pyridin-7-yloxy)methyl)-2-oxabicyclo[3.1.1]heptan-1- |
| | yl)methyl)isoquinolin-6-amine |
| 10961 | N5-((5-(((3-(trifluoromethyl)-[1,2,4]triazolo[4,3-a]pyridin-7-yl)oxy)methyl)-2- |
| | oxabicyclo[3.1.1]heptan-1-yl)methyl)isoquinoline-1,5-diamine |
| 10962 | 4-((1-(((1-aminoisoquinolin-5-yl)amino)methyl)-2-oxabicyclo[3.1.1]heptan-5- |
| | yl)methoxy)-1,5-dimethylpyrimidin-2(1H)-one |
| 10963 | N5-((5-((pyridazin-4-yloxy)methyl)-2-oxabicyclo[3.1.1]heptan-1- |
| | yl)methyl)isoquinoline-1,5-diamine |
| 10964 | 4-((1-(((1-aminoisoquinolin-5-yl)amino)methyl)-2-oxabicyclo[3.1.1]heptan-5- |
| | yl)methoxy)-1-ethyl-6-methylpyridin-2(1H)-one |
| 10965 | 1-((1-(((1-aminoisoquinolin-5-yl)amino)methyl)-2-oxabicyclo[3.1.1]heptan-5- |
| | yl)methyl)pyridazin-4(1H)-one |
| 10966 | 3-((1-(((1-aminoisoquinolin-5-yl)amino)methyl)-2-oxabicyclo[3.1.1]heptan-5- |
| | yl)methyl)pyridazin-4-ol |
| 10968 | N5-((5-((pyridin-3-yloxy)methyl)-2-oxabicyclo[3.1.1]heptan-1- |
| | yl)methyl)isoquinoline-1,5-diamine |
| 10969 | N5-((5-(([1,2,3]triazolo[1,5-a]pyridin-5-yloxy)methyl)-2-oxabicyclo[3.1.1]heptan- |
| | 1-yl)methyl)isoquinoline-1,5-diamine |
| 10970 | 3-chloro-N-((5-((imidazo[1,2-a]pyridin-7-yloxy)methyl)-2-oxabicyclo[3.1.1]heptan- |
| | 1-yl)methyl)-1H-indol-5-amine |
| 10971 | N5-((5-(((6-methylpyrimidin-4-yl)oxy)methyl)-2-oxabicyclo[3.1.1]heptan-1- |
| | yl)methyl)isoquinoline-1,5-diamine |
| 10972 | 7-((1-(((1-aminoisoquinolin-5-yl)amino)methyl)-2-oxabicyclo[3.1.1]heptan-5- |
| | yl)methoxy)-2,3-dihydroindolizin-5(1H)-one |
| 10973 | N5-((5-((benzo[d]oxazol-6-yloxy)methyl)-2-oxabicyclo[3.1.1]heptan-1- |
| | yl)methyl)isoquinoline-1,5-diamine |
| 10974 | N5-((5-(((2-methylpyrimidin-4-yl)oxy)methyl)-2-oxabicyclo[3.1.1]heptan-1- |
| | yl)methyl)isoquinoline-1,5-diamine |
| 10975 | 4-((1-(((1-aminoisoquinolin-5-yl)amino)methyl)-2-oxabicyclo[3.1.1]heptan-5- |
| | yl)methoxy)-1-(difluoromethyl)pyridin-2(1H)-one |
| 10976 | N5-((5-((pyrazin-2-yloxy)methyl)-2-oxabicyclo[3.1.1]heptan-1- |
| | yl)methyl)isoquinoline-1,5-diamine |
| 11001 | 4-((5-(((1-aminoisoquinolin-5-yl)amino)methyl)-2-oxabicyclo[3.1.1]heptan-1- |
| | yl)methoxy)-1-methylpyridin-2(1H)-one |
| 12001 | benzyl 1-(((1-aminoisoquinolin-5-yl)amino)methyl)-4-(((1-methyl-2-oxo-1,2- |
| | dihydropyridin-4-yl)oxy)methyl)-2-azabicyclo[2.1.1]hexane-2-carboxylate |
| 12007 | 4-((1-(((1-aminoisoquinolin-5-yl)amino)methyl)-2-azabicyclo[2.1.1]hexan-4- |
| | yl)methoxy)-1-methylpyridin-2(1H)-one |
| 12008 | phenyl 1-(((1-aminoisoquinolin-5-yl)amino)methyl)-4-(((1-methyl-2-oxo-1,2- |
| | dihydropyridin-4-yl)oxy)methyl)-2-azabicyclo[2.1.1]hexane-2-carboxylate |
| 12009 | 4-((2-acetyl-1-(((1-aminoisoquinolin-5-yl)amino)methyl)-2-azabicyclo[2.1.1]hexan- |
| | 4-yl)methoxy)-1-methylpyridin-2(1H)-one |
| 12010 | benzyl 1-(((1-aminoisoquinolin-6-yl)amino)methyl)-4-(((1-methyl-2-oxo-1,2- |
| | dihydropyridin-4-yl)oxy)methyl)-2-azabicyclo[2.1.1]hexane-2-carboxylate |
| 12011 | 4-((1-(((1-aminoisoquinolin-6-yl)amino)methyl)-2-azabicyclo[2.1.1]hexan-4- |
| | yl)methoxy)-1-methylpyridin-2(1H)-one |
| 12012 | benzyl 1-(((1-aminoisoquinolin-5-yl)amino)methyl)-4-(((2-methyl-3-oxoisoindolin- |
| | 5-yl)oxy)methyl)-2-azabicyclo[2.1.1]hexane-2-carboxylate |
| 12013 | methyl 1-(((1-aminoisoquinolin-6-yl)amino)methyl)-4-(((1-methyl-2-oxo-1,2- |
| | dihydropyridin-4-yl)oxy)methyl)-2-azabicyclo[2.1.1]hexane-2-carboxylate |
| 12014 | 4-((1-(((1-aminoisoquinolin-5-yl)amino)methyl)-2-(phenethylsulfonyl)-2- |
| | azabicyclo[2.1.1]hexan-4-yl)methoxy)-1-methylpyridin-2(1H)-one |
| 12015 | 2-chlorophenyl 1-(((1-aminoisoquinolin-5-yl)amino)methyl)-4-(((1-methyl-2-oxo- |
| | 1,2-dihydropyridin-4-yl)oxy)methyl)-2-azabicyclo[2.1.1]hexane-2-carboxylate |
| 12016 | 4-((1-(((1-aminoisoquinolin-5-yl)amino)methyl)-2-(3-phenylpropanoyl)-2- |
| | azabicyclo[2.1.1]hexan-4-yl)methoxy)-1-methylpyridin-2(1H)-one |
| 12017 | 1-(((1-aminoisoquinolin-5-yl)amino)methyl)-N-benzyl-4-(((1-methyl-2-oxo-1,2- |
| | dihydropyridin-4-yl)oxy)methyl)-2-azabicyclo[2.1.1]hexane-2-carboxamide |
| 12018 | benzyl 1-(((1-aminoisoquinolin-5-yl)amino)methyl)-4-(((2-methyl-1-oxoisoindolin- |
| | 5-yl)oxy)methyl)-2-azabicyclo[2.1.1]hexane-2-carboxylate |
| 12019 | 4-((1-(((1-aminoisoquinolin-5-yl)amino)methyl)-2-(3-phenylpropyl)-2- |
| | azabicyclo[2.1.1]hexan-4-yl)methoxy)-1-methylpyridin-2(1H)-one |
| 12020 | 3,4-difluorobenzyl 1-(((1-aminoisoquinolin-5-yl)amino)methyl)-4-(((1-methyl-2- |
| | oxo-1,2-dihydropyridin-4-yl)oxy)methyl)-2-azabicyclo[2.1.1]hexane-2-carboxylate |
| 12021 | pyridin-2-ylmethyl 1-(((1-aminoisoquinolin-5-yl)amino)methyl)-4-(((1-methyl-2- |
| | oxo-1,2-dihydropyridin-4-yl)oxy)methyl)-2-azabicyclo[2.1.1]hexane-2-carboxylate |
| 12022 | pyridin-3-ylmethyl 1-(((1-aminoisoquinolin-5-yl)amino)methyl)-4-(((1-methyl-2- |
| | oxo-1,2-dihydropyridin-4-yl)oxy)methyl)-2-azabicyclo[2.1.1]hexane-2-carboxylate |
| 12023 | 2-chlorobenzyl 1-(((1-aminoisoquinolin-5-yl)amino)methyl)-4-(((1-methyl-2-oxo- |
| | 1,2-dihydropyridin-4-yl)oxy)methyl)-2-azabicyclo[2.1.1]hexane-2-carboxylate |
| 12024 | naphthalen-2-ylmethyl 1-(((1-aminoisoquinolin-5-yl)amino)methyl)-4-(((1-methyl- |
| | 2-oxo-1,2-dihydropyridin-4-yl)oxy)methyl)-2-azabicyclo[2.1.1]hexane-2- |
| | carboxylate |
| 12025 | benzyl 1-(((2-chloro-1H-pyrrolo[2,3-b]pyridin-4-yl)amino)methyl)-4-(((1-methyl-2- |
| | oxo-1,2-dihydropyridin-4-yl)oxy)methyl)-2-azabicyclo[2.1.1]hexane-2-carboxylate |
| 12026 | (1-methyl-1H-pyrazol-4-yl)methyl 1-(((1-aminoisoquinolin-5-yl)amino)methyl)-4- |
| | (((1-methyl-2-oxo-1,2-dihydropyridin-4-yl)oxy)methyl)-2-azabicyclo[2.1.1]hexane- |
| | 2-carboxylate |
| 12027 | tert-butyl 1-(((1-aminoisoquinolin-5-yl)amino)methyl)-4-(((1-methyl-2-oxo-1,2- |
| | dihydropyridin-4-yl)oxy)methyl)-2-azabicyclo[2.1.1]hexane-2-carboxylate |
| 12028 | 4-((1-(((1-aminoisoquinolin-5-yl)amino)methyl)-2-(2-phenylacetyl)-2- |
| | azabicyclo[2.1.1]hexan-4-yl)methoxy)-1-methylpyridin-2(1H)-one |
| 12031 | 4-((1-(((1-aminoisoquinolin-5-yl)amino)methyl)-2-(4-phenylpyrimidin-2-yl)-2- |
| | azabicyclo[2.1.1]hexan-4-yl)methoxy)-1-methylpyridin-2(1H)-one |
| 12034 | 4-((1-(((1-aminoisoquinolin-5-yl)amino)methyl)-2-(2-(benzofuran-5-yl)acetyl)-2- |
| | azabicyclo[2.1.1]hexan-4-yl)methoxy)-1-methylpyridin-2(1H)-one |
| 12035 | 4-((1-(((1-aminoisoquinolin-5-yl)amino)methyl)-2-benzoyl-2- |
| | azabicyclo[2.1.1]hexan-4-yl)methoxy)-1-methylpyridin-2(1H)-one |
| 12036 | 1-(((1-aminoisoquinolin-5-yl)amino)methyl)-4-(((1-methyl-2-oxo-1,2- |
| | dihydropyridin-4-yl)oxy)methyl)-N-phenyl-2-azabicyclo[2.1.1]hexane-2- |
| | carboxamide |
| 12038 | 1-(((1-aminoisoquinolin-5-yl)amino)methyl)-N-benzyl-4-(((1,6-dimethyl-2-oxo-1,2- |
| | dihydropyridin-4-yl)oxy)methyl)-2-azabicyclo[2.1.1]hexane-2-carboxamide |
| 12039 | 4-((1-(((1-aminoisoquinolin-5-yl)amino)methyl)-2-(2-phenylacetyl)-2- |
| | azabicyclo[2.1.1]hexan-4-yl)methoxy)-1,6-dimethylpyridin-2(1H)-one |
| 12040 | 4-((2-(2-(1H-indol-4-yl)acetyl)-1-(((1-aminoisoquinolin-5-yl)amino)methyl)-2- |
| | azabicyclo[2.1.1]hexan-4-yl)methoxy)-1-methylpyridin-2(1H)-one |
| 12041 | 1-(((1-aminoisoquinolin-5-yl)amino)methyl)-N-benzyl-N-methyl-4-(((1-methyl-2- |
| | oxo-1,2-dihydropyridin-4-yl)oxy)methyl)-2-azabicyclo[2.1.1]hexane-2- |
| | carboxamide |
| 12042 | 4-((1-(((1-aminoisoquinolin-5-yl)amino)methyl)-2-(3-(3- |
| | methoxyphenyl)propanoyl)-2-azabicyclo[2.1.1]hexan-4-yl)methoxy)-1- |
| | methylpyridin-2(1H)-one |
| 12044 | 3-(3-(1-(((1-aminoisoquinolin-5-yl)amino)methyl)-4-(((1-methyl-2-oxo-1,2- |
| | dihydropyridin-4-yl)oxy)methyl)-2-azabicyclo[2.1.1]hexan-2-yl)-3- |
| | oxopropyl)benzonitrile |
| 12045 | tert-butyl 1-((isoquinolin-6-ylamino)methyl)-4-(((1-methyl-2-oxo-1,2- |
| | dihydropyridin-4-yl)oxy)methyl)-2-azabicyclo[2.1.1]hexane-2-carboxylate |
| 12046 | tert-butyl 4-(((1,6-dimethyl-2-oxo-1,2-dihydropyridin-4-yl)oxy)methyl)-1- |
| | ((isoquinolin-5-ylamino)methyl)-2-azabicyclo[2.1.1]hexane-2-carboxylate |
| 12047 | 4-((1-(((1-aminoisoquinolin-5-yl)amino)methyl)-2-(3-(isoquinolin-5-yl)propanoyl)- |
| | 2-azabicyclo[2.1.1]hexan-4-yl)methoxy)-1-methylpyridin-2(1H)-one |
| 12048 | 4-((1-(((1-aminoisoquinolin-5-yl)amino)methyl)-2-(3-(3- |
| | (difluoromethyl)phenyl)propanoyl)-2-azabicyclo[2.1.1]hexan-4-yl)methoxy)-1- |
| | methylpyridin-2(1H)-one |
| 12049 | 4-((1-(((1-aminoisoquinolin-5-yl)amino)methyl)-2-(3-(3-chlorophenyl)propanoyl)- |
| | 2-azabicyclo[2.1.1]hexan-4-yl)methoxy)-1-methylpyridin-2(1H)-one |
| 12050 | 4-((1-(((1-aminoisoquinolin-5-yl)amino)methyl)-2-(3-(3- |
| | (trifluoromethyl)phenyl)propanoyl)-2-azabicyclo[2.1.1]hexan-4-yl)methoxy)-1- |
| | methylpyridin-2(1H)-one |
| 12051 | 3-(3-(1-(((1-aminoisoquinolin-5-yl)amino)methyl)-4-(((1-methyl-2-oxo-1,2- |
| | dihydropyridin-4-yl) oxy)methyl)-2-azabicyclo[2.1.1]hexan-2-yl)-3- |
| | oxopropyl)benzoic acid |
| 12052 | 4-((1-(((1-aminoisoquinolin-5-yl)amino)methyl)-2-(3-(quinolin-5-yl)propanoyl)-2- |
| | azabicyclo[2.1.1]hexan-4-yl)methoxy)-1-methylpyridin-2(1H)-one |
| 12053 | 4-((1-(((1-aminoisoquinolin-5-yl)amino)methyl)-2-(3-(3-bromophenyl)propanoyl)- |
| | 2-azabicyclo[2.1.1]hexan-4-yl)methoxy)-1-methylpyridin-2(1H)-one |
| 12054 | 4-((1-(((1-aminoisoquinolin-5-yl)amino)methyl)-2-(3-(6-oxo-1,6-dihydropyridin-3- |
| | yl)propanoyl)-2-azabicyclo[2.1.1]hexan-4-yl)methoxy)-1-methylpyridin-2(1H)-one |
| 12055 | 4-((1-(((1-aminoisoquinolin-5-yl)amino)methyl)-2-(3-(quinolin-7-yl)propanoyl)-2- |
| | azabicyclo[2.1.1]hexan-4-yl)methoxy)-1-methylpyridin-2(1H)-one |
| 12056 | 4-((1-(((1-aminoisoquinolin-5-yl)amino)methyl)-2-(3-(3- |
| | (difluoromethoxy)phenyl)propanoyl)-2-azabicyclo[2.1.1]hexan-4-yl)methoxy)-1- |
| | methylpyridin-2(1H)-one |
| 12057 | 4-((1-(((1-aminoisoquinolin-5-yl)amino)methyl)-2-(benzo[d]oxazol-2-yl)-2- |
| | azabicyclo[2.1.1]hexan-4-yl)methoxy)-1-methylpyridin-2(1H)-one |
| 12058 | 1-(((1-aminoisoquinolin-5-yl)amino)methyl)-N-(2-fluorobenzyl)-4-(((1-methyl-2- |
| | oxo-1,2-dihydropyridin-4-yl)oxy)methyl)-2-azabicyclo[2.1.1]hexane-2- |
| | carboxamide |
| 12059 | 1-(((1-aminoisoquinolin-5-yl)amino)methyl)-N-(2-methoxyphenyl)-4-(((1-methyl- |
| | 2-oxo-1,2-dihydropyridin-4-yl)oxy)methyl)-2-azabicyclo[2.1.1]hexane-2- |
| | carboxamide |
| 12060 | 4-((1-(((1-aminoisoquinolin-5-yl)amino)methyl)-2-(5-phenylpyrimidin-2-yl)-2- |
| | azabicyclo[2.1.1]hexan-4-yl)methoxy)-1-methylpyridin-2(1H)-one |
| 12061 | 1-(((1-aminoisoquinolin-5-yl)amino)methyl)-N-(4-methoxyphenyl)-4-(((1-methyl- |
| | 2-oxo-1,2-dihydropyridin-4-yl)oxy)methyl)-2-azabicyclo[2.1.1]hexane-2- |
| | carboxamide |
| 12062 | 1-(((1-aminoisoquinolin-5-yl)amino)methyl)-N-(4-methoxybenzyl)-4-(((1-methyl- |
| | 2-oxo-1,2-dihydropyridin-4-yl)oxy)methyl)-2-azabicyclo[2.1.1]hexane-2- |
| | carboxamide |
| 12063 | tert-butyl 1-((isoquinolin-7-ylamino)methyl)-4-(((1-methyl-2-oxo-1,2- |
| | dihydropyridin-4-yl)oxy)methyl)-2-azabicyclo[2.1.1]hexane-2-carboxylate |
| 12064 | 4-((1-(((1-aminoisoquinolin-5-yl)amino)methyl)-2-(pyridin-3-yl)-2- |
| | azabicyclo[2.1.1]hexan-4-yl)methoxy)-1-methylpyridin-2(1H)-one |
| 12065 | 4-((1-(((1-aminoisoquinolin-5-yl)amino)methyl)-2-(pyridin-4-yl)-2- |
| | azabicyclo[2.1.1]hexan-4-yl)methoxy)-1-methylpyridin-2(1H)-one |
| 12067 | 1-(((1-aminoisoquinolin-5-yl)amino)methyl)-N-(2-methoxybenzyl)-4-(((1-methyl- |
| | 2-oxo-1,2-dihydropyridin-4-yl)oxy)methyl)-2-azabicyclo[2.1.1]hexane-2- |
| | carboxamide |
| 12068 | 1-(((1-aminoisoquinolin-5-yl)amino)methyl)-N-(2-fluorophenyl)-4-(((1-methyl-2- |
| | oxo-1,2-dihydropyridin-4-yl)oxy)methyl)-2-azabicyclo[2.1.1]hexane-2- |
| | carboxamide |
| 12069 | 1-(((1-aminoisoquinolin-5-yl)amino)methyl)-4-(((1-methyl-2-oxo-1,2- |
| | dihydropyridin-4-yl)oxy)methyl)-N-(3-(trifluoromethyl)benzyl)-2- |
| | azabicyclo[2.1.1]hexane-2-carboxamide |
| 12070 | 1-(((1-aminoisoquinolin-5-yl)amino)methyl)-N-(benzofuran-5-ylmethyl)-4-(((1- |
| | methyl-2-oxo-1,2-dihydropyridin-4-yl)oxy)methyl)-2-azabicyclo[2.1.1]hexane-2- |
| | carboxamide |
| 12071 | 4-((1-(((1-aminoisoquinolin-5-yl)amino)methyl)-2-(4-methoxybenzoyl)-2- |
| | azabicyclo[2.1.1]hexan-4-yl)methoxy)-1-methylpyridin-2(1H)-one |
| 12072 | 1-(((1-amino-6-fluoroisoquinolin-5-yl)amino)methyl)-N-benzyl-4-(((1-methyl-2- |
| | oxo-1,2-dihydropyridin-4-yl)oxy)methyl)-2-azabicyclo[2.1.1]hexane-2- |
| | carboxamide |
| 13001 | benzyl 4-(((1-aminoisoquinolin-5-yl)amino)methyl)-1-(((1-methyl-2-oxo-1,2- |
| | dihydropyridin-4-yl)oxy)methyl)-2-azabicyclo[2.1.1]hexane-2-carboxylate |
| 13002 | benzyl 4-(((1-aminoisoquinolin-5-yl)amino)methyl)-1-(3-(trifluoromethyl)-5,6,7,8- |
| | tetrahydro-[1,2,4]triazolo[4,3-a]pyrazine-7-carbonyl)-2-azabicyclo[2.1.1]hexane- |
| | 2-carboxylate |
| 13003 | 4-((2-acetyl-4-(((1-aminoisoquinolin-5-yl)amino)methyl)-2-azabicyclo[2.1.1]hexan- |
| | 1-yl)methoxy)-1-methylpyridin-2(1H)-one |
| 13004 | 2-chlorophenyl 4-(((1-aminoisoquinolin-5-yl)amino)methyl)-1-(((1-methyl-2-oxo- |
| | 1,2-dihydropyridin-4-yl)oxy)methyl)-2-azabicyclo[2.1.1]hexane-2-carboxylate |
| 13005 | tert-butyl 4-(((1-aminoisoquinolin-5-yl)amino)methyl)-1-(((1-methyl-2-oxo-1,2- |
| | dihydropyridin-4-yl)oxy)methyl)-2-azabicyclo[2.1.1]hexane-2-carboxylate |
| 13006 | phenyl 4-(((1-aminoisoquinolin-5-yl)amino)methyl)-1-(((1-methyl-2-oxo-1,2- |
| | dihydropyridin-4-yl)oxy)methyl)-2-azabicyclo[2.1.1]hexane-2-carboxylate |
| 13007 | 4-((4-(((1-aminoisoquinolin-5-yl)amino)methyl)-2-azabicyclo[2.1.1]hexan-1- |
| | yl)methoxy)-1-methylpyridin-2(1H)-one |
| 13008 | 4-((4-(((1-aminoisoquinolin-5-yl)amino)methyl)-2-(3-phenylpropanoyl)-2- |
| | azabicyclo[2.1.1]hexan-1-yl)methoxy)-1-methylpyridin-2(1H)-one |
| 13009 | pyridin-3-ylmethyl 4-(((1-aminoisoquinolin-5-yl)amino)methyl)-1-(((1-methyl-2- |
| | oxo-1,2-dihydropyridin-4-yl)oxy)methyl)-2-azabicyclo[2.1.1]hexane-2-carboxylate |
| 13010 | 4-((4-(((1-aminoisoquinolin-5-yl)amino)methyl)-2-(3-(pyridin-3-yl)propanoyl)-2- |
| | azabicyclo[2.1.1]hexan-1-yl)methoxy)-1-methylpyridin-2(1H)-one |
| 13011 | 4-(((1-aminoisoquinolin-5-yl)amino)methyl)-N-benzyl-N-methyl-1-(((1-methyl-2- |
| | oxo-1,2-dihydropyridin-4-yl)oxy)methyl)-2-azabicyclo[2.1.1]hexane-2- |
| | carboxamide |
| 13012 | 4-(((1-aminoisoquinolin-5-yl)amino)methyl)-N-benzyl-1-(((1-methyl-2-oxo-1,2- |
| | dihydropyridin-4-yl)oxy)methyl)-2-azabicyclo[2.1.1]hexane-2-carboxamide |
| 13013 | pyridin-2-ylmethyl 4-(((1-aminoisoquinolin-5-yl)amino)methyl)-1-(((1-methyl-2- |
| | oxo-1,2-dihydropyridin-4-yl)oxy)methyl)-2-azabicyclo[2.1.1]hexane-2-carboxylate |
| 13014 | 4-((4-(((1-aminoisoquinolin-5-yl)amino)methyl)-2-(phenethylsulfonyl)-2- |
| | azabicyclo[2.1.1]hexan-1-yl)methoxy)-1-methylpyridin-2(1H)-one |
| 13015 | 4-((4-(((1-aminoisoquinolin-5-yl)amino)methyl)-2-(3-phenylpropyl)-2- |
| | azabicyclo[2.1.1]hexan-1-yl)methoxy)-1-methylpyridin-2(1H)-one |
| 13016 | 4-((4-(((1-aminoisoquinolin-5-yl)amino)methyl)-2-(3-(pyridin-2-yl)propanoyl)-2- |
| | azabicyclo[2.1.1]hexan-1-yl)methoxy)-1-methylpyridin-2(1H)-one |
| 13017 | 4-((4-(((1-aminoisoquinolin-5-yl)amino)methyl)-2-(3-(2-chlorophenyl)propanoyl)- |
| | 2-azabicyclo[2.1.1]hexan-1-yl)methoxy)-1-methylpyridin-2(1H)-one |
| 13018 | 4-[[4-[[(1-aminoisoquinolin-5-yl)amino]methyl]-2-(1,3-benzoxazol-2-yl)-2- |
| | azabicyclo[2.1.1]hexan-1-yl]methoxy]-1-methylpyridin-2-one |
| 13019 | 4-(((1-aminoisoquinolin-5-yl)amino)methyl)-1-(((1-methyl-2-oxo-1,2- |
| | dihydropyridin-4-yl)oxy)methyl)-N-phenyl-2-azabicyclo[2.1.1]hexane-2- |
| | carboxamide |
| 13020 | 2-chlorobenzyl 4-(((1-aminoisoquinolin-5-yl)amino)methyl)-1-(((1-methyl-2-oxo- |
| | 1,2-dihydropyridin-4-yl)oxy)methyl)-2-azabicyclo[2.1.1]hexane-2-carboxylate |
| 13021 | 4-((4-(((1-aminoisoquinolin-5-yl)amino)methyl)-2-(2-phenylacetyl)-2- |
| | azabicyclo[2.1.1]hexan-1-yl)methoxy)-1-methylpyridin-2(1H)-one |
| 13022 | (1-methyl-1H-pyrazol-4-yl)methyl 4-(((1-aminoisoquinolin-5-yl)amino)methyl)-1- |
| | (((1-methyl-2-oxo-1,2-dihydropyridin-4-yl)oxy)methyl)-2-azabicyclo[2.1.1]hexane- |
| | 2-carboxylate |

TABLE 15
¹H NMR data of examples (solvent d6 DMSO unless otherwise indicated)

| Example | |
|---|---|
| No | NMR write-up |
| 1001 | 1.75 (6H, s), 4.04 (2H, s), 5.87 (1H, t, J = 5.9 Hz), 6.47 (2H, s), 6.56 (1H, dd, J = 7.4, 2.5 Hz), |
| | 6.65 (1H, d, J = 7.7 Hz), 6.87 (1H, d, J = 2.5 Hz), 7.11-7.16 (1H, m), 7.19 (1H, t, J = 8.0 Hz), |
| | 7.31 (1H, d, J = 8.3 Hz), 7.36 (1H, d, J = 1.3 Hz), 7.63-7.75 (2H, m), 8.35 (1H, dd, J = 7.4, 0.6 Hz) |
| 1002 | (CDCl₃) 1.74 (6H, s), 2.04-2.17 (2H, m), 2.96 (1H, dd, J = 16.8, 5.8 Hz), 3.08 (1H, dd, J = |
| | 16.7, 4.7 Hz), 3.32 (2H, d, J = 3.6 Hz), 3.47-3.59 (2H, m), 3.84-3.96 (2H, m), 4.05-4.14 |
| | (1H, m), 4.22 (1H, s), 5.18 (2H, s), 6.71 (1H, dd, J = 7.7, 0.8 Hz), 6.79 (1H, d, J = 1.3 Hz), 6.93 |
| | (1H, dd, J = 6.2, 1.0 Hz), 6.99 (1H, d, J = 1.3 Hz), 7.11 (1H, dt, J = 8.5, 1.0 Hz), 7.34 (1H, t, J = |
| | 8.0 Hz), 7.91 (1H, d, J = 6.2 Hz) |
| 1002.1 | 1.61 (6H, s), 1.96-2.07 (2H, m), 2.69 (1H, dd, J = 16.6, 5.5 Hz), 2.94 (1H, dd, J = 16.6, 4.6 |
| | Hz), 3.28 (2H, d, J = 5.8 Hz), 3.45 (2H, d, J = 2.8 Hz), 3.82-3.96 (3H, m), 5.81 (1H, t, J = 5.9 |
| | Hz), 6.46 (2H, s), 6.63 (1H, d, J = 7.5 Hz), 6.78 (1H, d, J = 1.3 Hz), 6.96 (1H, d, J = 1.3 Hz), |
| | 7.09-7.15 (1H, m), 7.19 (1H, t, J = 8.0 Hz), 7.30 (1H, d, J = 8.3 Hz), 7.70 (1H, d, J = 6.1 Hz) |
| 1002.2 | 1.61 (6H, s), 1.96-2.05 (2H, m), 2.69 (1H, dd, J = 16.5, 5.6 Hz), 2.94 (1H, dd, J = 16.6, 4.6 |
| | Hz), 3.28 (2H, d, J = 5.8 Hz), 3.45 (2H, d, J = 2.8 Hz), 3.82-3.96 (3H, m), 5.81 (1H, t, J = 5.7 |
| | Hz), 6.46 (2H, s), 6.62 (1H, d, J = 7.6 Hz), 6.78 (1H, d, J = 1.3 Hz), 6.96 (1H, d, J = 1.3 Hz), |
| | 7.12 (1H, d, J = 5.9 Hz), 7.19 (1H, t, J = 8.0 Hz), 7.30 (1H, d, J = 8.3 Hz), 7.70 (1H, d, J = 6.1 Hz) |
| 1004 | 1.69 (6H, s), 2.56 (2H, s), 2.80 (2H, dd, J = 6.3, 4.7 Hz), 3.28 (2H, d, J = 5.6 Hz), 3.56 (2H, s), |
| | 3.91 (2H, t, J = 5.5 Hz), 5.81 (1H, t, J = 5.9 Hz), 6.47 (2H, s), 6.63 (1H, d, J = 7.6 Hz), 6.80 (1H, |
| | d, J = 1.2 Hz), 7.00 (1H, d, J = 1.2 Hz), 7.13 (1H, d, J = 6.1 Hz), 7.19 (1H, t, J = 8.0 Hz), 7.30 |
| | (1H, d, J = 8.3 Hz), 7.70 (1H, d, J = 6.1 Hz) |
| 1005.1 | 0.89 (3H, d, J = 6.1 Hz), 0.94 (3H, d, J = 6.0 Hz), 1.45 (1H, d, J = 9.0 Hz), 1.51 (1H, d, J = 9.0 |
| | Hz), 1.58 (6H, s), 2.36-2.40 (1H, m), 2.41 (1H, d, J = 7.9 Hz), 2.46-2.49 (1H, m), 2.51-2.53 |
| | (2H, m), 2.55 (2H, d, J = 9.9 Hz), 2.68 (1H, dd, J = 9.3, 2.6 Hz), 3.13 (1H, s), 3.27 (2H, d, J = |
| | 5.7 Hz), 5.80 (1H, t, J = 5.8 Hz), 6.48 (2H, s), 6.61 (1H, d, J = 7.7 Hz), 7.09-7.15 (1H, m), 7.18 |
| | (1H, t, J = 8.0 Hz), 7.30 (1H, d, J = 8.3 Hz), 7.70 (1H, d, J = 6.0 Hz) |
| 1006 | (CD₃CN) 1.83 (6H, s), 2.31 (3H, d, J = 0.9 Hz), 3.40 (2H, d, J = 4.1 Hz), 4.06 (2H, s), 4.86 (1H, |
| | s), 5.57 (2H, s), 6.46 (1H, dd, J = 7.4, 2.5 Hz), 6.72 (1H, d, J = 2.5 Hz), 6.77-6.81 (1H, m), |
| | 7.05 (1H, dd, J = 6.1, 1.0 Hz), 7.20 (1H, dt, J = 8.3, 1.0 Hz), 7.30 (1H, s), 7.34 (1H, t, J = 8.0 |
| | Hz), 7.83 (1H, d, J = 6.2 Hz), 8.03 (1H, dd, J = 7.4, 0.7 Hz) |
| 1009 | 1.73 (6H, s), 3.35 (2H, d, J = 5.9 Hz), 4.04 (2H, s), 6.12 (1H, d, J = 5.5 Hz), 6.47 (1H, t, J = 6.2 |
| | Hz), 6.53-6.60 (2H, m), 6.86 (1H, d, J = 2.5 Hz), 7.03 (1H, d, J = 3.4 Hz), 7.36 (1H, s), 7.71 |
| | (1H, s), 7.77 (1H, d, J = 5.5 Hz), 8.35 (1H, d, J = 7.4 Hz), 11.07 (1H, s) |
| 1010 | 1.72 (6H, s), 4.04 (2H, s), 6.16 (1H, d, J = 5.8 Hz), 6.54-6.59 (3H, m), 6.86 (1H, d, J = 2.5 Hz), |
| | 7.36 (1H, d, J = 1.3 Hz), 7.71 (1H, s), 7.74 (1H, d, J = 5.7 Hz), 8.35 (1H, dd, J = 7.4, 0.7 Hz), |
| | 11.93 (1H, s) |
| 1011 | 1.78 (6H, s), 3.27 (2H, d, J = 5.1 Hz), 4.07 (2H, s), 6.47 (1H, t, J = 5.5 Hz), 6.58 (1H, dd, J = |
| | 7.4, 2.5 Hz), 6.65 (1H, s), 6.89 (1H, d, J = 2.5 Hz), 7.14 (1H, dd, J = 8.9, 2.2 Hz), 7.37 (1H, s), |
| | 7.39 (1H, d, J = 5.9 Hz), 7.68-7.76 (2H, m), 8.16 (1H, d, J = 5.8 Hz), 8.36 (1H, d, J = 7.4 Hz), |
| | 8.84 (1H, s) |
| 1012 | 1.72 (6H, s), 2.29 (3H, s), 3.32 (2H, s, obscured by H2O), 4.03 (2H, s), 6.08 (1H, d, J = 5.5 |
| | Hz), 6.16-6.26 (2H, m), 6.56 (1H, dd, J = 7.4, 2.5 Hz), 6.86 (1H, d, J = 2.5 Hz), 7.36 (1H, s), |
| | 7.67 (1H, d, J = 5.5 Hz), 7.71 (1H, s), 8.35 (1H, d, J = 7.4 Hz), 10.89 (1H, s) |
| 1013 | 1.88 (6H, s), 4.12 (2H, s), 4.16 (2H, s), 6.63 (1H, dd, J = 7.4, 2.4 Hz), 6.76 (2H, s), 6.92 (1H, d, |
| | J = 2.3 Hz), 7.07 (1H, d, J = 7.7 Hz), 7.13 (1H, d, J = 5.8 Hz), 7.35 (1H, t, J = 8.1 Hz), 7.38 (1H, |
| | d, J = 0.6 Hz), 7.70-7.74 (2H, m), 7.80 (1H, d, J = 5.9 Hz), 8.38 (1H, d, J = 7.4 Hz) |
| 1014 | (CDCl₃) 1.78 (6H, s), 1.86-1.97 (2H, m), 2.90-2.99 (2H, m), 4.02 (2H, s), 5.28 (2H, d, J = |
| | 18.4 Hz), 6.53 (1H, dd, J = 7.4, 2.4 Hz), 6.84 (1H, d, J = 2.5 Hz), 7.15 (1H, dd, J = 6.2, 1.0 Hz), |
| | 7.36-7.45 (2H, m), 7.48 (2H, dd, J = 7.2, 1.3 Hz), 7.68 (1H, dt, J = 8.3, 1.3 Hz), 7.93 (1H, dd, |
| | J = 7.4, 0.7 Hz), 7.96 (1H, d, J = 6.2 Hz) |
| 1015 | 2.20 (6H, d, J = 8.0 Hz), 3.83-4.01 (2H, m), 4.05 (2H, s), 4.16 (2H, s), 4.62 and 4.85 (total |
| | 2H, each s), 6.81 (2H, d, J = 6.6 Hz), 6.90 (1H, d, J = 12.7 Hz), 7.09 (1H, d, J = 7.8 Hz), 7.13 |
| | (1H, s), 7.18 (1H, d, J = 5.9 Hz), 7.36 (1H, t, J = 8.1 Hz), 7.73 (1H, d, J = 8.4 Hz), 7.80 (1H, d, J = |
| | 5.9 Hz) |
| 1016 | 2.08 (6H, s), 2.95-3.20 (1H, m), 3.81-4.05 (5H, m), 4.58-4.79 (2H, m), 5.86-5.93 (1H, m), |
| | 6.52 (2H, s), 6.65 (1H, d, J = 7.7 Hz), 6.87 (1H, d, J = 9.3 Hz), 7.09-7.23 (3H, m), 7.33 (1H, d, |
| | J = 8.3 Hz), 7.72 (1H, d, J = 6.2 Hz) |
| 1017 | 1.76 (6H, s), 3.96 (2H, s), 5.88 (1H, t, J = 5.7 Hz), 6.48 (2H, s), 6.66 (1H, d, J = 7.7 Hz), 7.00 |
| | (1H, dd, J = 9.7, 2.3 Hz), 7.14 (1H, d, J = 6.1 Hz), 7.20 (1H, t, J = 8.0 Hz), 7.31 (1H, d, J = 8.3 |
| | Hz), 7.44 (1H, d, J = 9.9 Hz), 7.48 (1H, s), 7.72 (1H, d, J = 6.1 Hz), 7.80 (1H, s), 8.23 (1H, d, J = |
| | 2.3 Hz). 2H masked by water peak. |
| 1018 | 1.76 (6H, s), 4.17 (2H, s), 5.86 (1H, t, J = 5.9 Hz), 6.46 (2H, s), 6.58 (1H, d, J = 7.5 Hz), 6.65 |
| | (1H, d, J = 7.7 Hz), 6.72 (1H, t, J = 7.1 Hz), 7.14 (1H, d, J = 6.1 Hz), 7.19 (1H, t, J = 8.0 Hz), |
| | 7.30 (1H, d, J = 8.3 Hz), 7.44 (1H, s), 7.71 (1H, d, J = 6.0 Hz), 7.89 (1H, s), 8.11 (1H, d, J = 6.7 |
| | Hz). 2H hidden by water peak (confirmed by COSY). |
| 1019 | 1.71 (6H, s), 3.31 (3H,s), 3.95 (2H, s), 5.74 (1H, d, J = 2.7 Hz), 5.86-5.90 (2H, m), 6.48 (2H, |
| | s), 6.64 (1H, d, J = 7.7 Hz), 7.14 (1H, d, J = 6.2 Hz), 7.20 (1H, t, J = 8.0 Hz), 7.31 (1H, d, J = 8.3 |
| | Hz), 7.53 (1H, d, J = 7.7 Hz), 7.71 (1H, d, J = 6.1 Hz). 2H masked by water peak. |
| 1101 | 1.74 (6H, s), 4.05 (2H, s), 5.87 (1H, t, J = 5.8 Hz), 6.44 (1H, t, J = 2.2 Hz), 6.47 (2H, s), 6.65 |
| | (1H, d, J = 7.7 Hz), 7.14 (1H, d, J = 6.1 Hz), 7.20 (1H, t, J = 8.0 Hz), 7.27 (1H, d, J = 2.3 Hz), |
| | 7.31 (1H, d, J = 8.3 Hz), 7.43 (1H, t, J = 2.9 Hz), 7.71 (1H, d, J = 6.1 Hz), 8.05 (1H, d, J = 2.5 |
| | Hz), 11.04 (1H, s). 2H masked by water peak |
| 1105 | 1.73 (6H, s), 3.32 (2H, s), 3.98 (2H, s), 5.89 (1H, t, J = 5.8 Hz), 6.51 (2H, s), 6.63-6.67 (1H, |
| | m), 6.78 (1H, s), 6.97 (1H, s), 7.14 (1H, dd, J = 6.4, 0.9 Hz), 7.20 (1H, t, J = 8.0 Hz), 7.31 (1H, |
| | d, J = 8.3 Hz), 7.46 (1H, s), 7.71 (1H, d, J = 6.1 Hz), 8.06 (1H, s), 12.21 (1H, s) |
| 1109 | 1.72 (6H, s), 3.32 (2H, d, J = 5.6 Hz), 4.05 (2H, s), 4.27 (2H, s), 5.88 (1H, t, J = 6.0 Hz), 6.49 |
| | (2H, s), 6.64 (1H, d, J = 7.7 Hz), 7.10-7.15 (3H, m), 7.19 (1H, t, J = 8.0 Hz), 7.31 (1H, d, J = |
| | 8.3 Hz), 7.40-7.47 (1H, m), 7.71 (1H, d, J = 6.0 Hz), 8.53 (1H, s) |
| 1110 | 1.76 (6H, s), 3.32 (2H, s), 4.13 (2H, s), 5.73 (1H, t, J = 5.8 Hz), 6.53 (2H, s), 6.65 (1H, d, J = |
| | 7.7 Hz), 6.81 (1H, dd, J = 7.5, 2.6 Hz), 7.14-7.27 (3H, m), 7.31 (1H, d, J = 8.2 Hz), 7.70 (1H, d, |
| | J = 6.1 Hz), 8.30 (1H, s), 8.74 (1H, d, J = 7.5 Hz) |
| 1113 | 1.64 (6H, s), 3.28 (2H, s), 3.86 (2H, s), 5.79 (1H, t, J = 5.7 Hz), 6.27 (1H, d, J = 2.2 Hz), 6.36- |
| | 6.39 (3H, m), 6.57 (1H, d, J = 7.7 Hz), 6.97 (1H, d, J = 8.2 Hz), 7.06 (1H, d, J = 6.1 Hz), 7.12 |
| | (1H, t, J = 8.0 Hz), 7.23 (1H, d, J = 8.2 Hz), 7.64 (1H, d, J = 6.1 Hz), 10.21 (1H, s). 2H masked |
| | by water peak |
| 1118 | 1.63 (6H, s), 2.63 (1H, dd, J = 16.5, 2.7 Hz), 3.01 (1H, dd, J = 16.5, 6.6 Hz), 3.28 (2H, d, J = |
| | 5.5 Hz), 3.40-3.48 (2H, m), 3.82 (1H, dd, J = 11.7, 2.6 Hz), 4.14 (1H, dd, J = 11.7, 5.9 Hz), |
| | 4.64 (1H, tt, J = 5.8, 2.6 Hz), 5.83 (1H, t, J = 5.9 Hz), 6.48 (2H, s), 6.62 (1H, d, J = 7.7 Hz), 6.85 |
| | (1H, d, J = 1.4 Hz), 7.03 (1H, d, J = 1.2 Hz), 7.10-7.14 (1H, m), 7.18 (1H, t, J = 8.0 Hz), 7.30 |
| | (1H, d, J = 8.3 Hz), 7.70 (1H, d, J = 6.1 Hz) |
| 1125 | 1.74 (6H, s), 3.28 (2H, s), 4.02 (2H, s), 5.87 (1H, s), 6.49 (2H, s), 6.61-6.76 (2H, m), 6.87 |
| | (1H, s), 7.14 (1H, d, J = 6.2 Hz), 7.20 (1H, t, J = 8.0 Hz), 7.31 (1H, d, J = 8.3 Hz), 7.57 (1H, d, J = |
| | 8.8 Hz), 7.71 (1H, d, J = 6.1 Hz), 7.90 (1H, s), 12.75 (1H, s) |
| 1126 | 1.52 (6H, s), 3.26 (2H, d, J = 5.8 Hz), 4.11 (2H, s), 5.27 (1H, dd, J = 10.1, 2.2 Hz), 5.39 (1H, |
| | dd, J = 17.3, 2.2 Hz), 5.84 (1H, t, J = 5.8 Hz), 6.21-6.22 (2H, m), 6.45 (2H, s), 6.59 (1H, d, J = |
| | 7.7 Hz), 6.96 (1H, d, J = 0.7 Hz), 7.07 (1H, d, J = 0.8 Hz), 7.10 (1H, d, J = 6.2 Hz), 7.16 (1H, t, J = |
| | 8.0 Hz), 7.29 (1H, d, J = 8.2 Hz), 7.69 (1H, d, J = 6.1 Hz), 7.90-7.98 (1H, m) |
| 1127 | 1.59 (6H, s), 3.27 (2H, d, J = 5.8 Hz), 3.94 (2H, s), 5.80 (1H, d, J = 2.1 Hz), 5.86 (1H, t, J = 5.7 |
| | Hz), 6.12 (1H, dd, J = 7.5, 2.1 Hz), 6.46 (2H, s), 6.59 (1H, d, J = 7.7 Hz), 7.10 (1H, d, J = 5.7 |
| | Hz), 7.17 (1H, t, J = 8.0 Hz), 7.23 (1H, d, J = 2.4 Hz), 7.29 (1H, d, J = 8.3 Hz), 7.39 (1H, d, J = |
| | 2.1 Hz), 7.69 (1H, d, J = 6.1 Hz), 7.88 (1H, d, J = 7.7 Hz) |
| 1129 | 1.76 (6H, s), 3.17 (2H, d, J = 5.2 Hz), 4.06 (2H, s), 5.41 (1H, t, J = 5.7 Hz), 6.58 (1H, dd, J = |
| | 7.4, 2.5 Hz), 6.88 (1H, d, J = 2.5 Hz), 6.91 (1H, d, J = 2.6 Hz), 7.37 (1H, s), 7.41 (1H, d, J = 2.8 |
| | Hz), 7.72 (1H, s), 7.89 (1H, d, J = 2.6 Hz), 8.36 (1H, d, J = 7.4 Hz), 11.42 (1H, s) |
| 1130 | 1.59 (6H, s), 1.95-2.05 (2H, m), 2.68 (1H, dd, J = 16.6, 5.6 Hz), 2.94 (1H, dd, J = 16.5, 4.6 |
| | Hz), 3.30 (2H, d, J = 6.0 Hz), 3.41-3.48 (2H, m), 3.90 (3H, td, J = 6.2, 2.5 Hz), 6.14 (1H, d, J = |
| | 5.8 Hz), 6.53 (1H, s), 6.57 (1H, s), 6.78 (1H, d, J = 1.2 Hz), 6.96 (1H, d, J = 1.2 Hz), 7.73 (1H, |
| | d, J = 5.6 Hz), 11.93 (1H, s) |
| 1131 | (CDCl₃)2.27 (6H, s), 4.11 (2H, s), 4.84 (1H, s), 5.21 (2H, s), 6.90 (1H, dd, J = 6.2, 0.9 Hz), 7.00 |
| | (1H, td, J = 10.1, 2.3 Hz), 7.09 (1H, dd, J = 7.8, 0.9 Hz), 7.19 (1H, d, J = 8.3 Hz), 7.38 (1H, t, J = |
| | 8.0 Hz), 7.47-7.61 (3H, m), 7.68 (1H, dd, J = 5.6, 2.3 Hz), 7.90 (1H, d, J = 6.2 Hz) |
| 1132 | 2.07 (6H, s), 3.44 (2H, d, J = 5.7 Hz), 5.91 (1H, t, J = 5.8 Hz), 6.49 (2H, s), 6.67 (1H, d, J = 7.7 |
| | Hz), 7.16 (1H, d, J = 6.2 Hz), 7.21 (1H, t, J = 8.0 Hz), 7.33 (1H, d, J = 8.2 Hz), 7.72 (1H, d, J = |
| | 6.0 Hz), 7.78 (1H, t, J = 51.7 Hz), 7.90 (1H, dd, J = 9.6, 1.4 Hz), 7.99 (1H, dd, J = 9.6, 0.6 Hz), |
| | 9.03 (1H, s), 9.34 (1H, s) |
| 1133 | 1.68 (s, 6H), 3.25-3.32 (m, 3H), 4.01 (s, 2H), 4.23 (s, 2H), 5.83 (t, J = 5.8 Hz, 1H), 6.47 (s, |
| | 2H), 6.60 (dd, J = 7.7, 0.9 Hz, 1H), 6.93 (dd, J = 8.4, 2.3 Hz, 1H), 7.00-7.05 (m, 1H), 7.09 |
| | (dd, J = 6.3, 0.9 Hz, 1H), 7.15 (t, J = 8.0 Hz, 1H), 7.23-7.30 (m, 1H), 7.47 (d, J = 8.4 Hz, 1H), |
| | 7.66 (d, J = 6.1 Hz, 1H), 8.22 (s, 1H) |
| 1134 | (CDCl₃) 1.70 (6H, s), 2.26 (3H, s), 3.31 (5H, d, J = 4.8 Hz), 3.92 (2H, s), 5.64 (1H, d, J = 2.8 |
| | Hz), 5.79-5.91 (2H, m), 6.46 (2H, s), 6.64 (1H, d, J = 7.7 Hz), 7.13 (1H, d, J = 6.1 Hz), 7.19 |
| | (1H, t, J = 8.0 Hz), 7.30 (1H, d, J = 8.3 Hz), 7.71 (1H, d, J = 6.0 Hz) |
| 1135 | 1.72-1.78 (6H, m), 3.33-3.37 (2H, m), 4.09 (2H, s), 5.83-5.90 (1H, m), 6.46 (2H, s), 6.65 |
| | (2H, d, J = 7.5 Hz), 7.01 (1H, s), 7.14 (1H, d, J = 4.7 Hz), 7.19 (1H, t, J = 7.0 Hz), 7.31 (1H, d, J = |
| | 7.7 Hz), 7.71 (1H, dd, J = 6.0, 2.0 Hz), 8.35-8.43 (1H, m), 9.01 (1H, d, J = 1.4 Hz) |
| 2020 | 1.61 (2H, dd, J = 4.4, 1.7 Hz), 1.85 (2H, dd, J = 4.4, 1.7 Hz), 3.58 (2H, d, J = 5.9 Hz), 3.71 (2H, |
| | s), 4.25 (2H, s), 5.99 (1H, t, J = 5.9 Hz), 6.48 (2H, s), 6.59 (1H, dd, J = 7.4, 2.5 Hz), 6.68 (1H, |
| | d, J = 7.7 Hz), 6.93 (1H, d, J = 2.5 Hz), 7.17 (1H, d, J = 6.1 Hz), 7.21 (1H, t, J = 8.0 Hz), 7.32 |
| | (1H, d, J = 8.3 Hz), 7.37 (1H, d, J = 1.3 Hz), 7.68-7.76 (2H, m), 8.36 (1H, d, J = 7.4 Hz) |
| 2022 | 1.62 (2H, dd, J = 4.5, 1.7 Hz), 1.85 (2H, dd, J = 4.4, 1.7 Hz), 3.59 (2H, d, J = 5.9 Hz), 3.71 (2H, |
| | s), 4.17 (2H, s), 6.00 (1H, t, J = 6.0 Hz), 6.48 (2H, s), 6.69 (1H, d, J = 7.7 Hz), 7.02 (1H, dd, J = |
| | 9.7, 2.4 Hz), 7.13-7.27 (2H, m), 7.33 (1H, d, J = 8.3 Hz), 7.42-7.51 (2H, m), 7.72 (1H, d, J = |
| | 6.1 Hz), 7.81 (1H, t, J = 0.9 Hz), 8.24-8.30 (1H, m) |
| 2204 | 1.63 (2H, dd, J = 4.5, 1.7 Hz), 1.87 (2H, dd, J = 4.6, 1.6 Hz), 3.59 (2H, d, J = 5.8 Hz), 3.71 (2H, |
| | s), 4.56 (2H, s), 6.06 (1H, t, J = 6.0 Hz), 6.69-6.81 (3H, m), 7.17-7.27 (3H, m), 7.36 (1H, d, J = |
| | 8.3 Hz), 7.46-7.74 (2H, m), 8.38 (1H, d, J = 9.9 Hz) |
| 2205 | 1.57 (2H, dd, J = 4.5, 1.7 Hz), 1.80 (2H, dd, J = 4.5, 1.7 Hz), 3.31 (3H, s), 3.56 (2H, d, J = 5.8 |
| | Hz), 3.68 (2H, s), 4.14 (2H, s), 5.79 (1H, d, J = 2.8 Hz), 5.90 (1H, dd, J = 7.5, 2.7 Hz), 5.98 (1H, |
| | t, J = 6.0 Hz), 6.48 (2H, s), 6.67 (1H, d, J = 7.7 Hz), 7.11-7.17 (1H, m), 7.20 (1H, t, J = 8.0 Hz), |
| | 7.32 (1H, d, J = 8.3 Hz), 7.53 (1H, d, J = 7.6 Hz), 7.72 (1H, d, J = 6.0 Hz) |
| 2206 | 1.63 (2H, dd, J = 4.5, 1.7 Hz), 1.83-1.93 (2H, m), 3.59 (2H, d, J = 5.8 Hz), 3.72 (2H, s), 4.40 |
| | (2H, s), 6.02 (1H, t, J = 6.0 Hz), 6.54 (2H, s), 6.69 (1H, d, J = 7.7 Hz), 6.95 (1H, dd, J = 7.6, 2.4 |
| | Hz), 7.15-7.19 (1H, m), 7.22 (1H, t, J = 8.0 Hz), 7.34 (1H, d, J = 8.3 Hz), 7.39 (1H, dd, J = 2.4, |
| | 0.7 Hz), 7.72 (1H, d, J = 6.1 Hz), 8.45 (1H, d, J = 7.5 Hz) |
| 2207 | 1.62 (2H, dd, J = 4.4, 1.7 Hz), 1.86 (2H, dd, J = 4.5, 1.7 Hz), 3.58 (2H, d, J = 5.9 Hz), 3.71 (2H, |
| | s), 4.35 (2H, s), 6.00 (1H, t, J = 6.0 Hz), 6.49 (2H, s), 6.68 (1H, d, J = 7.7 Hz), 6.84 (1H, dd, J = |
| | 7.5, 2.6 Hz), 7.14-7.28 (3H, m), 7.33 (1H, d, J = 8.3 Hz), 7.72 (1H, d, J = 6.1 Hz), 8.31 (1H, s), |
| | 8.74 (1H, d, J = 7.5 Hz) |
| 2208 | 1.58 (2H, dd, J = 4.4, 1.7 Hz), 1.77-1.88 (2H, m), 3.56 (2H, d, J = 5.8 Hz), 3.69 (2H, s), 4.62 |
| | (2H, s), 5.98 (1H, t, J = 6.0 Hz), 6.48 (2H, s), 6.67 (1H, d, J = 7.7 Hz), 7.16 (1H, d, J = 6.1 Hz), |
| | 7.20 (1H, t, J = 8.0 Hz), 7.32 (1H, d, J = 8.3 Hz), 7.72 (1H, d, J = 6.1 Hz), 9.10 (2H, s) |
| 2210 | 1.62 (2H, dd, J = 4.4, 1.7 Hz), 1.85 (2H, dd, J = 4.5, 1.7 Hz), 3.58 (2H, d, J = 5.9 Hz), 3.71 (2H, |
| | s), 4.29 (2H, s), 5.99 (1H, t, J = 6.0 Hz), 6.48 (2H, s), 6.68 (1H, d, J = 7.7 Hz), 7.14-7.25 (2H, |
| | m), 7.33 (1H, d, J = 8.3 Hz), 7.45 (1H, dd, J = 9.7, 2.4 Hz), 7.69-7.77 (2H, m), 8.38 (1H, s), |
| | 8.70 (1H, dd, J = 2.5, 0.8 Hz) |
| 2211 | 1.59 (2H, dd, J = 4.5, 1.7 Hz), 1.82 (2H, dd, J = 4.6, 1.6 Hz), 3.05 (3H, s), 3.57 (2H, d, J = 5.9 |
| | Hz), 3.70 (2H, s), 4.25 (2H, s), 4.35 (2H, s), 5.97 (1H, t, J = 6.0 Hz), 6.47 (2H, s), 6.67 (1H, d, J = |
| | 7.7 Hz), 7.09-7.17 (3H, m), 7.20 (1H, t, J = 8.0 Hz), 7.32 (1H, d, J = 8.3 Hz), 7.44 (1H, d, J = |
| | 8.2, 0.8 Hz), 7.72 (1H, d, J = 6.1 Hz) |
| 2212 | 1.63 (2H, dd, J = 4.2, 1.1 Hz), 1.85 (2H, d, J = 4.4 Hz), 2.41 (3H, s), 3.59 (2H, d, J = 5.7 Hz), |
| | 3.72 (2H, s), 4.25 (2H, s), 6.00 (1H, t, J = 6.0 Hz), 6.51 (2H, s), 6.69 (1H, d, J = 7.8 Hz), 6.99 |
| | (1H, dd, J = 9.8, 2.2 Hz), 7.15-7.23 (2H, m), 7.28-7.34 (2H, m), 7.42 (1H, d, J = 9.7 Hz), |
| | 7.72 (1H, d, J = 6.1 Hz), 7.84 (1H, d, J = 2.1 Hz) |
| 4401 | 1.98 (4H, tdd, J = 11.6, 9.5, 6.2 Hz), 2.77 (2H, dt, J = 14.5, 7.2 Hz), 3.29 (2H, dd, J = 7.4, 5.4 |
| | Hz), 4.08 (2H, d, J = 7.0 Hz), 5.92 (1H, t, J = 5.4 Hz), 6.47 (2H, s), 6.57-6.64 (2H, m), 6.93 |
| | (1H, d, J = 2.5 Hz), 7.15 (1H, d, J = 6.1 Hz), 7.22 (1H, t, J = 8.0 Hz), 7.32 (1H, d, J = 8.3 Hz), |
| | 7.38 (1H, d, J = 1.3 Hz), 7.67-7.75 (2H, m), 8.38 (1H, d, J = 7.4 Hz) |
| 5003 | 1.66 (2H, dt, J = 11.8, 9.0 Hz), 2.18-2.29 (2H, m), 2.56-2.74 (2H, m, J = 8.1 Hz), 3.21 (2H, t, |
| | J = 6.1 Hz), 3.98 (2H, d, J = 6.3 Hz), 5.88 (1H, t, J = 5.4 Hz), 6.47 (2H, d, J = 4.4 Hz), 6.55- |
| | 6.63 (2H, m), 6.89 (1H, d, J = 2.5 Hz), 7.14 (1H, dd, J = 6.3, 0.9 Hz), 7.21 (1H, t, J = 8.0 Hz), |
| | 7.31 (1H, d, J = 8.3 Hz), 7.37 (1H, d, J = 1.3 Hz), 7.67-7.75 (2H, m), 8.36 (1H, dd, J = 7.4, 0.6 Hz) |
| 6601 | (CDCl₃) 1.82-1.97 (4H, m), 3.63 (2H, d, J = 5.2 Hz), 3.91 (2H, s), 4.28 (2H, s), 4.66 (1H, s), |
| | 5.10 (2H, s), 6.52 (1H, dd, J = 7.4, 2.4 Hz), 6.75 (1H, d, J = 7.7 Hz), 6.86 (1H, d, J = 2.4 Hz), |
| | 7.00 (1H, d, J = 6.1 Hz), 7.15 (1H, d, J = 8.3 Hz), 7.36 (1H, t, J = 8.0 Hz), 7.42 (1H, s), 7.49 (1H, |
| | d, J = 1.4 Hz), 7.94 (2H, dd, J = 6.8, 5.5 Hz) |
| 6602 | 1.60 (2H, dd, J = 4.6, 1.7 Hz), 1.86 (2H, dd, J = 4.4, 1.7 Hz), 3.55 (2H, d, J = 5.8 Hz), 3.74 (2H, |
| | s), 4.24 (2H, s), 5.94 (1H, t, J = 6.0 Hz), 6.52 (2H, s), 6.70 (1H, d, J = 7.6 Hz), 7.01 (1H, dd, J = |
| | 9.7, 2.4 Hz), 7.11 (1H, d, J = 6.1 Hz), 7.21 (1H, t, J = 8.0 Hz), 7.34 (1H, d, J = 8.3 Hz), 7.45 (1H, |
| | dt, J = 9.7, 0.8 Hz), 7.49 (1H, d, J = 1.1 Hz), 7.72 (1H, d, J = 6.1 Hz), 7.77-7.85 (1H, m), 8.28 |
| | (1H, dd, J = 2.5, 0.8 Hz) |
| 6605 | 1.56 (2H, dd, J = 4.3, 1.3 Hz), 1.83 (2H, d, J = 5.1 Hz), 2.30 (3H, s), 3.55 (2H, d, J = 5.8 Hz), |
| | 3.72 (2H, s), 4.31 (2H, s), 6.13 (1H, d, J = 5.6 Hz), 6.23 (1H, s), 6.33 (1H, t, J = 6.3 Hz), 6.58 |
| | (1H, dd, J = 7.4, 2.5 Hz), 6.92 (1H, d, J = 2.4 Hz), 7.37 (1H, d, J = 1.0 Hz), 7.69 (1H, d, J = 5.5 |
| | Hz), 7.72 (1H, s), 8.36 (1H, J = 7.4 Hz), 10.93 (1H, s) |
| 6606 | 1.56 (2H, dd, J = 4.2, 1.2 Hz), 1.84 (2H, d, J = 4.8 Hz), 3.57 (2H, d, J = 6.2 Hz), 3.72 (2H, s), |
| | 4.32 (2H, s), 6.22 (1H, d, J = 5.8 Hz), 6.57 (1H, d, J = 2.9 Hz), 6.59 (1H, s), 6.67 (1H, s), 6.92 |
| | (1H, d, J = 2.4 Hz), 7.37 (1H, d, J = 1.2 Hz), 7.72 (1H, s), 7.76 (1H, J = 5.6 Hz), 8.36 (1H, d, J = |
| | 7.4 Hz), 11.92 (1H, s) |
| 6608 | 1.61 (2H, dd, J = 4.6, 1.7 Hz), 1.88 (2H, dd, J = 4.5, 1.7 Hz), 3.54 (2H, d, J = 5.9 Hz), 3.74 (2H, |
| | s), 4.65 (2H, s), 5.93 (1H, t, J = 6.0 Hz), 6.51 (2H, s), 6.69 (1H, d, J = 7.7 Hz), 7.07-7.12 (1H, |
| | m), 7.18-7.24 (2H, m), 7.33 (1H, d, J = 8.3 Hz), 7.53 (1H, t, J = 51.4 Hz), 7.72 (1H, d, J = 5.9 |
| | Hz), 8.37 (1H, d, J = 9.9 Hz) |
| 6609 | 1.55 (2H, dd, J = 4.5, 1.7 Hz), 1.80 (2H, dd, J = 4.5, 1.7 Hz), 3.31 (10H, s), 3.53 (2H, d, J = 5.9 |
| | Hz), 3.68 (2H, s), 4.22 (2H, s), 5.79 (1H, d, J = 2.8 Hz), 5.86-5.96 (2H, m), 6.50 (2H, s), 6.68 |
| | (1H, d, J = 7.7 Hz), 7.10 (1H, d, J = 6.1 Hz), 7.20 (1H, t, J = 8.0 Hz), 7.33 (1H, d, J = 8.3 Hz), |
| | 7.53 (1H, d, J = 7.5 Hz), 7.72 (1H, d, J = 6.0 Hz) |
| 6610 | 1.43-1.49 (2H, m), 1.67 (2H, d, J = 4.9 Hz), 1.90 (1H, dddd, J = 13.5, 9.1, 6.6, 2.1 Hz), 1.98- |
| | 2.10 (1H, m), 2.59-2.74 (2H, m), 3.49 (2H, d, J = 5.9 Hz), 3.57 (2H, s), 3.68-3.82 (2H, m), |
| | 3.87-3.96 (2H, m), 3.99-4.08 (1H, m), 5.86 (1H, t, J = 6.0 Hz), 6.50 (2H, s), 6.66 (1H, d, J = |
| | 7.7 Hz), 6.77 (1H, d, J = 1.3 Hz), 6.92 (1H, d, J = 1.3 Hz), 7.08 (1H, d, J = 6.1 Hz), 7.19 (1H, t, J = |
| | 8.0 Hz), 7.32 (1H, d, J = 8.3 Hz), 7.71 (1H, d, J = 6.1 Hz) |
| 6611 | 1.57 (2H, dd, J = 4.3, 1.8 Hz), 1.84 (2H, d, J = 4.7 Hz), 3.57 (2H, d, J = 6.2 Hz), 3.72 (2H, s), |
| | 4.24 (2H, s), 6.22 (1H, d, J = 5.7 Hz), 6.59 (1H, s), 6.69 (1H, s), 7.02 (1H, dd, J = 9.7, 2.4 Hz), |
| | 7.45 (1H, d, J = 9.7 Hz), 7.49 (1H, d, J = 1.1 Hz), 7.76 (1H, d, J = 5.6 Hz), 7.81 (1H, s), 8.28 |
| | (1H, d, J = 2.4 Hz) azaindole NH not observed |
| 6612 | 1.60 (dd, J = 4.5, 1.7 Hz, 2H), 1.86 (dd, J = 4.5, 1.7 Hz, 2H), 3.52-3.59 (m, 2H), 3.73 (s, 2H), |
| | 4.57 (s, 2H), 5.92 (t, J = 6.1 Hz, 1H), 6.50 (s, 2H), 6.69 (d, J = 7.7 Hz, 1H), 6.86 (d, J = 9.7 Hz, |
| | 1H), 7.10 (d, J = 6.1 Hz, 1H), 7.21 (dd, J = 9.4, 6.6 Hz, 1H), 7.33 (d, J = 8.3 Hz, 1H), 7.59 (d, J = |
| | 1.2 Hz, 1H), 7.72 (dd, J = 6.2, 2.3 Hz, 1H), 7.98 (d, J = 9.7 Hz, 1H), 8.02 (d, J = 1.1 Hz, 1H) |
| 6613 | 1.60 (2H, dd, J = 4.4, 1.7 Hz), 1.86 (2H, dd, J = 4.4, 1.7 Hz), 3.55 (2H, d, J = 5.9 Hz), 3.74 (2H, |
| | s), 4.34 (2H, s), 5.95 (1H, t, J = 6.0 Hz), 6.58 (2H, s), 6.66-6.74 (2H, m), 6.89-7.19 (3H, m), |
| | 7.22 (1H, t, J = 8.0 Hz), 7.35 (1H, d, J = 8.3 Hz), 7.72 (1H, d, J = 6.1 Hz), 8.04 (1H, t, J = 2.1 |
| | Hz), 8.40 (1H, d, J = 7.5 Hz) |
| 6614 | 1.59 (2H, dd, J = 4.4, 1.4 Hz), 1.85 (2H, d, J = 4.5, 1.0 Hz), 3.54 (2H, d, J = 5.7 Hz), 3.71 (2H, |
| | s), 4.63 (2H, s), 5.91 (1H, t, J = 5.9 Hz), 6.52 (2H, s), 6.58 (1H, d, J = 7.2 Hz), 6.68 (1H, d, J = |
| | 7.7 Hz), 7.10 (1H, d, J = 6.2 Hz), 7.21 (1H, t, J = 8.0 Hz), 7.33 (1H, d, J = 8.3 Hz), 7.39 (1H, d, J = |
| | 1.4 Hz), 7.65 (1H, d, J = 1.5 Hz), 7.71 (1H, d, J = 6.1 Hz), 8.21 (1H, s), 8.73 (1H, d, J = 7.2 |
| | Hz) acid proton missing |
| 6615 | 1.61 (2H, dd, J = 4.3, 1.2 Hz), 1.87 (2H, d, J = 4.4, 0.8 Hz), 3.55 (2H, d, J = 5.9 Hz), 3.74 (2H, |
| | s), 4.60 (2H, s), 5.92 (1H, t, J = 6.0 Hz), 6.49 (2H, s), 6.69 (1H, d, J = 7.7 Hz), 7.08 (1H, d, J = |
| | 4.7 Hz), 7.10 (1H, s), 7.21 (1H, t, J = 8.0 Hz), 7.33 (1H, d, J = 8.3 Hz), 7.72 (1H, d, J = 6.1 Hz), |
| | 8.11 (1H, d, J = 9.8 Hz), 8.70 (1H, s) |
| 6616 | 1.57 (2H, dd, J = 4.5, 1.7 Hz), 1.83 (2H, dd, J = 4.5, 1.7 Hz), 3.53 (2H, d, J = 5.8 Hz), 3.72 (2H, |
| | s), 4.47 (2H, s), 5.90 (1H, t, J = 6.0 Hz), 6.49 (2H, s), 6.69 (1H, d, J = 7.7 Hz), 7.09 (1H, d, J = |
| | 6.1 Hz), 7.20 (1H, t, J = 8.0 Hz), 7.33 (1H, d, J = 8.3 Hz), 7.71 (1H, d, J = 6.1 Hz), 7.78 (1H, d, J = |
| | 1.0 Hz), 8.02 (1H, d, J = 0.9 Hz), 8.24 (1H, d, J = 1.4 Hz), 8.81 (1H, d, J = 1.3 Hz) |
| 6617 | 1.59 (2H, dd, J = 4.4, 1.7 Hz), 1.85 (2H, dd, J = 4.7, 1.6 Hz), 2.42 (3H, d, J = 0.9 Hz), 3.55 (2H, |
| | d, J = 5.9 Hz), 3.73 (2H, s), 4.21 (2H, s), 5.94 (1H, t, J = 6.1 Hz), 6.50 (2H, s), 6.69 (1H, d, J = |
| | 7.7 Hz), 6.86 (1H, dd, J = 2.4, 1.2 Hz), 7.07-7.13 (1H, m), 7.21 (1H, t, J = 8.0 Hz), 7.33 (1H, d, |
| | J = 8.3 Hz), 7.44 (1H, d, J = 1.1 Hz), 7.72 (1H, d, J = 6.1 Hz), 7.78 (1H, d, J = 1.1 Hz), 8.10- |
| | 8.14 (1H, m) |
| 6618 | 1.58 (2H, dd, J = 4.5, 1.7 Hz), 1.84 (2H, dd, J = 4.6, 1.7 Hz), 3.54 (2H, d, J = 5.6 Hz), 3.72 (2H, |
| | s), 4.28 (2H, s), 4.33 (2H, s), 5.94 (1H, t, J = 6.0 Hz), 6.59 (2H, s), 6.70 (1H, d, J = 7.7 Hz), 7.01 |
| | (1H, dd, J = 8.4, 2.3 Hz), 7.08-7.16 (2H, m), 7.21 (1H, t, J = 8.0 Hz), 7.34 (1H, d, J = 8.3 Hz), |
| | 7.53 (1H, d, J = 8.4 Hz), 7.71 (1H, d, J = 6.1 Hz), 8.27 (1H, s), 8.31 (1H, s) formic acid OH was |
| | not observed |
| 6619 | 1.79 (2H, dd, J = 4.2, 1.1 Hz), 2.05 (2H, d, J = 4.1 Hz), 3.42 (2H, s), 3.84 (2H, s), 4.43 (2H, s), |
| | 5.59 (1H, s), 6.82 (2H, s), 7.03 (1H, dd, J = 9.2, 2.1 Hz), 7.19-7.23 (2H, m), 7.41-7.46 (3H, |
| | m), 7.55 (1H, d, J = 1.7 Hz), 7.78 (1H, s), 7.81 (1H, d, J = 8.4 Hz), 7.88 (1H, d, J = 5.9 Hz), |
| | 8.39 (2H, s) two acid protons missing |
| 6620 | 1.61 (2H, dd, J = 4.4, 1.7 Hz), 1.88 (2H, d, J = 4.5 Hz), 3.56 (2H, d, J = 5.8 Hz), 3.75 (2H, s), 4.35 |
| | (2H, s), 5.94 (1H, t, J = 6.1 Hz), 6.51 (2H, s), 6.70 (1H, d, J = 7.7 Hz), 7.10 (1H, d, J = 6.2 Hz), |
| | 7.21 (1H, t, J = 8.0 Hz), 7.31-7.41 (2H, m), 7.56-7.84 (2H, m), 7.87 (1H, d, J = 9.9 Hz), 8.12 |
| | (1H, d, J = 2.2 Hz) |
| 6621 | 1.58 (2H, dd, J = 4.4, 1.7 Hz), 1.84 (2H, dd, J = 4.4, 1.7 Hz), 3.02 (3H, s), 3.54 (2H, d, J = 5.9 |
| | Hz), 3.72 (2H, s), 4.35 (4H, d, J = 11.4 Hz), 5.92 (1H, t, J = 6.0 Hz), 6.50 (2H, s), 6.69 (1H, d, J = |
| | 7.7 Hz), 7.00 (1H, dd, J = 8.4, 2.3 Hz), 7.10 (1H, d, J = 6.1 Hz), 7.14 (1H, d, J = 2.2 Hz), 7.21 |
| | (1H, t, J = 8.0 Hz), 7.33 (1H, d, J = 8.3 Hz), 7.52 (1H, d, J = 8.3 Hz), 7.72 (1H, d, J = 6.1 Hz) |
| 6622 | 1.63 (2H, dd, J = 4.2, 1.3 Hz), 1.88 (2H, dd, J = 4.4, 0.9 Hz), 2.19 (3H, s), 3.56 (2H, d, J = 5.9 |
| | Hz), 3.76 (2H, s), 4.22 (2H, s), 5.90 (1H, t, J = 6.0 Hz), 6.51 (2H, s), 6.72 (1H, d, J = 7.7 Hz), |
| | 7.11 (1H, d, J = 6.2 Hz), 7.22 (1H, t, J = 8.0 Hz), 7.32-7.35 (2H, m), 7.39 (1H, d, J = 0.6 Hz), |
| | 7.71-7.73 (2H, m), 8.19 (1H, s) |
| 6623 | (CDCl₃) 1.85 (2H, dd, J = 4.7, 1.5 Hz), 1.92 (3H, d, J = 4.8 Hz), 3.60 (2H, s), 3.90 (2H, s), 4.53 |
| | (2H, s), 6.77-6.84 (2H, m), 6.97 (1H, dd, J = 6.5, 0.9 Hz), 7.16 (1H, d, J = 8.3 Hz), 7.40 (1H, t, |
| | J = 8.0 Hz), 7.52-7.61 (2H, m), 7.74 (1H, d, J = 6.5 Hz), 8.80 (1H, d, J = 1.3 Hz) NH2 protons |
| | not observed |
| 6624 | 1.61 (2H, dd, J = 4.3, 1.3 Hz), 1.88 (2H, d, J = 5.3 Hz), 2.41 (3H, s), 3.56 (2H, d, J = 5.8 Hz), |
| | 3.75 (2H, s), 4.32 (2H, s), 5.93 (1H, t, J = 5.9 Hz), 6.54 (2H, s), 6.71 (1H, d, J = 7.7 Hz), 6.99 |
| | (1H, dd, J = 9.7, 2.3 Hz), 7.11 (1H, d, J = 6.2 Hz), 7.22 (1H, t, J = 8.0 Hz), 7.29 (1H, d, J = 0.7 |
| | Hz), 7.34 (1H, d, J = 7.7 Hz), 7.43 (1H, d, J = 9.9 Hz), 7.72 (1H, d, J = 6.1 Hz), 7.83 (1H, d, J = |
| | 2.1 Hz), 8.19 (2H, s) two acid protons missing |
| 6625 | 1.60 (2H, dd, J = 4.3, 1.7 Hz), 1.86 (2H, dd, J = 4.3, 1.7 Hz), 3.55 (2H, d, J = 5.9 Hz), 3.74 (2H, |
| | s), 4.30 (2H, s), 5.93 (1H, t, J = 6.0 Hz), 6.51 (2H, s), 6.70 (1H, d, J = 7.7 Hz), 7.01 (1H, dd, J = |
| | 9.9, 2.4 Hz), 7.11 (1H, d, J = 6.2 Hz), 7.21 (1H, t, J = 8.0 Hz), 7.28 (1H, d, J = 7.2 Hz), 7.34 (1H, |
| | d, J = 8.3 Hz), 7.42 (1H, dd, J = 9.8, 2.0 Hz), 7.72 (1H, d, J = 6.1 Hz), 7.91 (1H, d, J = 2.3 Hz) |
| 6626 | 1.59 (2H, dd, J = 4.3, 1.4 Hz), 1.84 (2H, dd, J = 4.3, 1.0 Hz), 2.46 (3H, s), 3.55 (2H, d, J = 5.7 |
| | Hz), 3.74 (2H, s), 4.29 (2H, s), 5.93 (1H, t, J = 6.0 Hz), 6.53 (2H, s), 6.70 (1H, d, J = 7.7 Hz), |
| | 7.11 (1H, d, J = 6.2 Hz), 7.21 (1H, t, J = 8.0 Hz), 7.29-7.34 (2H, m), 7.45 (1H, d, J = 9.6 Hz), |
| | 7.61 (1H, d, J = 0.9 Hz), 7.71 (1H, d, J = 6.3 Hz), 7.76 (1H, s), 8.19 (1H, s) one acid proton |
| | missing |
| 6627 | 1.61 (2H, dd, J = 4.5, 1.7 Hz), 1.87 (2H, dd, J = 4.4, 1.7 Hz), 3.56 (2H, d, J = 5.9 Hz), 3.75 (2H, |
| | s), 4.27 (2H, s), 5.94 (1H, t, J = 6.1 Hz), 6.51 (2H, d, J = 3.2 Hz), 6.70 (1H, d, J = 7.8 Hz), 7.11 |
| | (1H, dd, J = 6.5, 2.0 Hz), 7.18-7.26 (2H, m), 7.34 (1H, d, J = 8.4 Hz), 7.58 (1H, d, J = 9.8 Hz), |
| | 7.73 (1H, d, J = 6.1 Hz), 8.31 (1H, dd, J = 2.4, 0.8 Hz), 8.36 (1H, t, J = 1.0 Hz) |
| 6628 | 1.57 (2H, dd, J = 4.5, 1.7 Hz), 1.84 (2H, dd, J = 4.4, 1.7 Hz), 3.05 (3H, s), 3.54 (2H, d, J = 5.8 |
| | Hz), 3.72 (2H, s), 4.34 (4H, d, J = 12.0 Hz), 5.91 (1H, t, J = 6.1 Hz), 6.50 (2H, s), 6.69 (1H, d, J = |
| | 7.6 Hz), 7.08-7.18 (3H, m), 7.21 (1H, t, J = 8.0 Hz), 7.33 (1H, d, J = 8.3 Hz), 7.44 (1H, d, J = |
| | 8.2 Hz), 7.72 (1H, d, J = 6.0 Hz) |
| 6629 | 8 1.60 (2H, dd, J = 4.4, 1.3 Hz), 1.86 (2H, dd, J = 4.5, 0.8 Hz), 3.56 (2H, d, J = 7.2 Hz), 3.73 (2H, |
| | s), 4.38 (2H, s), 5.96 (1H, t, J = 5.7 Hz), 6.63 (2H, s), 6.66-6.72 (2H, m), 7.07-7.13 (2H, m), |
| | 7.19-7.24 (1H, m), 7.34 (1H, d, J = 8.2 Hz), 7.72 (1H, d, J = 6.1 Hz), 8.18 (1H, s), 8.39 (1H, d, |
| | J = 7.3 Hz), 9.02 (1H, s) |
| 6630 | 1.55-1.65 (2H, m), 1.87 (2H, dd, J = 4.5, 1.7 Hz), 3.55 (2H, d, J = 5.9 Hz), 3.75 (2H, s), 4.31 |
| | (2H, s), 5.94 (1H, t, J = 6.0 Hz), 6.51 (2H, s), 6.69 (1H, d, J = 7.7 Hz), 7.10 (1H, d, J = 6.1 Hz), |
| | 7.21 (1H, t, J = 8.0 Hz), 7.33 (1H, d, J = 8.3 Hz), 7.49 (1H, dd, J = 2.3, 1.0 Hz), 7.62 (1H, d, J = |
| | 1.1 Hz), 7.72 (1H, d, J = 6.0 Hz), 7.99 (1H, d, J = 1.2 Hz), 8.61 (1H, d, J = 2.3 Hz) |
| 6631 | 1.60 (2H, dd, J = 4.5, 1.7 Hz), 1.83-1.88 (2H, m), 3.56 (2H, d, J = 5.9 Hz), 3.74 (2H, s), 3.89 |
| | (3H, s), 4.21 (2H, s), 5.96 (1H, t, J = 6.1 Hz), 6.41 (1H, d, J = 2.0 Hz), 6.51 (2H, d, J = 5.1 Hz), |
| | 6.69 (1H, d, J = 7.7 Hz), 7.11 (1H, d, J = 6.3 Hz), 7.21 (1H, td, J = 7.8, 4.0 Hz), 7.34 (1H, d, J = |
| | 8.2 Hz), 7.39 (1H, d, J = 1.1 Hz), 7.69-7.75 (1H, m), 7.77 (1H, d, J = 1.1 Hz), 7.89 (1H, d, J = |
| | 2.0 Hz) |
| 6633 | 1.59 (2H, dd, J = 4.5, 1.7 Hz), 1.84 (2H, dd, J = 4.6, 1.7 Hz), 2.29 (3H, d, J = 0.7 Hz), 3.58 (2H, |
| | d, J = 5.6 Hz), 3.73 (2H, s), 4.22 (2H, s), 6.28 (1H, t, J = 6.0 Hz), 6.90 (1H, d, J = 7.9 Hz), 6.98 |
| | (1H, dd, J = 9.7, 2.4 Hz), 7.28-7.40 (3H, m), 7.49 (1H, d, J = 8.3 Hz), 7.56 (1H, t, J = 0.8 Hz), |
| | 7.59-7.73 (3H, m), 8.14 (2H, s), 8.21 (1H, dd, J = 2.5, 0.8 Hz) |
| 6634 | 1.61 (2H, dd, J = 4.5, 1.7 Hz), 1.86 (2H, dd, J = 4.4, 1.7 Hz), 3.56 (2H, d, J = 5.7 Hz), 3.74 (2H, |
| | s), 4.26 (2H, s), 5.99 (1H, t, J = 6.1 Hz), 6.69 (2H, s), 6.73 (1H, d, J = 7.8 Hz), 7.13 (1H, d, J = |
| | 6.2 Hz), 7.18 (1H, dd, J = 9.9, 2.2 Hz), 7.23 (1H, t, J = 8.0 Hz), 7.36 (1H, d, J = 8.3 Hz), 7.66- |
| | 7.74 (2H, m), 8.16 (1H, s), 8.25 (1H, d, J = 2.2 Hz), 9.09 (1H, s) |
| 6635 | 1.46 (2H, dd, J = 4.6, 1.7 Hz), 1.67 (2H, d, J = 5.0 Hz), 3.46 (2H, d, J = 5.8 Hz), 3.55 (2H, s), |
| | 3.83 (2H, s), 5.84 (1H, t, J = 6.0 Hz), 6.51 (2H, s), 6.63 (1H, d, J = 7.7 Hz), 7.04-7.14 (3H, m), |
| | 7.18 (1H, t, J = 8.0 Hz), 7.31 (1H, d, J = 8.3 Hz), 7.67 (2H, d, J = 8.2 Hz), 8.30 (1H, s) |
| 6636 | 1.59 (2H, dd, J = 4.4, 1.7 Hz), 1.85 (2H, dd, J = 4.4, 1.7 Hz), 3.52-3.58 (2H, m), 3.73 (2H, s), |
| | 4.45 (2H, s), 5.93 (1H, t, J = 6.1 Hz), 6.53 (2H, d, J = 5.6 Hz), 6.70 (1H, d, J = 7.7 Hz), 7.07- |
| | 7.14 (1H, m), 7.17-7.25 (1H, m), 7.29-7.36 (2H, m), 7.69-7.77 (2H, m), 8.30 (2H, s) |
| 6637 | 1.57 (2H, dd, J = 4.4, 1.7 Hz), 1.83 (2H, dd, J = 4.5, 1.7 Hz), 2.83 (2H, t, J = 6.6 Hz), 3.29- |
| | 3.32 (2H, m), 3.54 (2H, d, J = 5.9 Hz), 3.71 (2H, s), 4.30 (2H, s), 5.93 (1H, t, J = 6.0 Hz), 6.51 |
| | (2H, s), 6.69 (1H, d, J = 7.7 Hz), 6.82-6.91 (2H, m), 7.10 (1H, d, J = 6.1 Hz), 7.21 (1H, t, J = |
| | 8.0 Hz), 7.33 (1H, d, J = 8.3 Hz), 7.69-7.77 (3H, m) |
| 6638 | 1.61 (2H, dd, J = 4.5, 1.7 Hz), 1.87 (2H, dd, J = 4.5, 1.7 Hz), 3.55 (2H, d, J = 5.9 Hz), 3.73 (2H, |
| | s), 4.60 (2H, s), 5.94 (1H, t, J = 6.0 Hz), 6.51 (2H, s), 6.69 (1H, d, J = 7.7 Hz), 7.05 (1H, d, J = |
| | 9.8 Hz), 7.10 (1H, d, J = 6.2 Hz), 7.21 (1H, t, J = 8.0 Hz), 7.33 (1H, d, J = 8.3 Hz), 7.72 (1H, d, J = |
| | 6.1 Hz), 8.24 (1H, dd, J = 9.8, 0.8 Hz), 9.39 (1H, d, J = 0.8 Hz) |
| 6639 | 0.60-0.64 (2H, m), 0.96-1.00 (2H, m), 1.61 (2H, dd, J = 4.2, 1.3 Hz), 1.97 (2H, d, J = 5.1 |
| | Hz), 1.92-1.99 (1H, m), 3.56 (2H, d, J = 5.8 Hz), 3.75 (2H, s), 4.34 (2H, s), 5.95 (1H, t, J = 5.9 |
| | Hz), 6.55 (2H, s), 6.71 (1H, d, J = 7.8 Hz), 7.02 (1H, dd, J = 9.7, 2.3 Hz), 7.11 (1H, d, J = 6.2 |
| | Hz), 7.20-7.24 (2H, m), 7.34 (1H, d, J = 8.3 Hz), 7.42 (1H, d, J = 9.7 Hz), 7.72 (1H, d, J = 6.1 |
| | Hz), 7.99 (1H, d, J = 2.0 Hz), 8.25 (2H, s) two acid protons missing |
| 6640 | 1.57 (2H, d, J = 3.4 Hz), 1.83 (2H, d, J = 3.5 Hz), 2.90 (2H, t, J = 6.5 Hz), 2.97 (3H, s), 3.48 (2H, |
| | t, J = 6.6 Hz), 3.55 (2H, d, J = 4.7 Hz), 3.71 (2H, s), 4.30 (2H, s), 6.09 (1H,s), 6.77 (1H, d, J = |
| | 7.8 Hz), 6.83-6.88 (2H, m), 7.02 (2H, s), 7.19 (1H, d, J = 6.3 Hz), 7.27 (1H, t, J = 7.9 Hz), |
| | 7.40 (1H, d, J = 8.2 Hz), 7.69 (1H, d, J = 6.2 Hz), 7.75 (1H, d, J = 8.6 Hz) |
| 6641 | 1.29 (6H, d, J = 6.9 Hz), 1.60 (2H, dd, J = 4.4, 1.7 Hz), 1.86 (2H, dd, J = 4.4, 1.7 Hz), 3.46 (1H, |
| | p, J = 6.9 Hz), 3.55 (2H, d, J = 6.0 Hz), 3.74 (2H, s), 4.21 (2H, s), 5.95 (1H, t, J = 6.0 Hz), 6.51 |
| | (2H, s), 6.69 (1H, d, J = 7.7 Hz), 6.79 (1H, d, J = 2.2 Hz), 7.11 (1H, d, J = 6.2 Hz), 7.21 (1H, t, J = |
| | 8.0 Hz), 7.33 (1H, d, J = 8.3 Hz), 7.44 (1H, d, J = 1.1 Hz), 7.72 (1H, d, J = 6.1 Hz), 7.78 (1H, |
| | d, J = 1.1 Hz), 8.11 (1H, d, J = 2.3 Hz) |
| 6642 | 1.57 (2H, dd, J = 4.5, 1.7 Hz), 1.84 (2H, dd, J = 4.5, 1.7 Hz), 3.54 (2H, d, J = 5.9 Hz), 3.72 (2H, |
| | s), 4.30 (4H, d, J = 20.6 Hz), 5.93 (1H, t, J = 6.0 Hz), 6.51 (2H, s), 6.69 (1H, d, J = 7.7 Hz), 7.10 |
| | (1H, d, J = 6.1 Hz), 7.12-7.17 (2H, m), 7.21 (1H, t, J = 8.0 Hz), 7.33 (1H, d, J = 8.3 Hz), 7.44 |
| | (1H, d), 7.71 (1H, d, J = 6.0 Hz), 8.54 (1H, s) |
| 6643 | 1.59 (2H, dd, J = 4.3, 1.2 Hz), 1.85 (2H, d, J = 4.5 Hz), 3.45 (3H, s), 3.61 (2H, d, J = 5.9 Hz), |
| | 3.73 (2H, s), 4.37 (2H, s), 6.48-6.53 (2H, m), 7.02-7.06 (2H, m), 7.12 (1H, d, J = 2.4 Hz), |
| | 7.41-7.49 (3H, m), 7.58-7.62 (2H, m), 8.09 (1H, d, J = 8.9 Hz), 8.35 (2H, s), 12.90 (1H, s) |
| 6644 | 1.61 (2H, dd, J = 4.5, 1.7 Hz), 1.87 (2H, dd, J = 4.5, 1.7 Hz), 3.56 (2H, d, J = 5.9 Hz), 3.75 (2H, |
| | s), 4.32 (2H, s), 5.93 (1H, t, J = 6.0 Hz), 6.50 (2H, s), 6.70 (1H, d, J = 7.7 Hz), 7.11 (1H, d, J = |
| | 6.1 Hz), 7.18-7.28 (2H, m), 7.34 (1H, d, J = 8.3 Hz), 7.44 (1H, t, J = 53.3 Hz), 7.61-7.64 (1H, |
| | m), 7.72 (1H, d, J = 6.1 Hz), 7.85 (1H, t, J = 2.0 Hz), 8.07 (1H, dd, J = 2.4, 0.8 Hz) |
| 6645 | 1.44 (2H, dd, J = 4.5, 1.7 Hz), 1.62 (2H, dd, J = 4.5, 1.6 Hz), 3.47 (2H, d, J = 5.9 Hz), 3.50 (2H, |
| | s), 4.45 (2H, s), 5.84-5.91 (1H, m), 6.19-6.21 (1H, m), 6.48 (2H, s), 6.63 (1H, d, J = 7.4 Hz), |
| | 7.06 (1H, d, J = 5.9 Hz), 7.19 (1H, t, J = 8.0 Hz), 7.32 (1H, d, J = 8.4 Hz), 7.40 (1H, dd, J = 1.8, |
| | 0.7 Hz), 7.65 (1H, dd, J = 2.2, 0.7 Hz), 7.70 (1H, d, J = 6.1 Hz) |
| 6646 | 1.45 (2H, dd, J = 4.5, 1.6 Hz), 1.62 (2H, dd, J = 4.5, 1.6 Hz), 3.46-3.51 (4H, m), 4.32 (2H, s), |
| | 5.90 (1H, t, J = 5.8 Hz), 6.52 (2H, s), 6.64 (1H, d, J = 7.5 Hz), 6.86 (1H, s), 7.07 (2H, d, J = 6.4 |
| | Hz), 7.19 (1H, t, J = 8.0 Hz), 7.32 (1H, d, J = 8.3 Hz), 7.55 (1H, s), 7.70 (1H, d, J = 6.1 Hz) |
| 6647 | 1.48 (2H, dd, J = 4.6, 1.7 Hz), 1.64-1.70 (2H, m), 3.48 (2H, d, J = 6.0 Hz), 3.54 (2H, s), 4.78 |
| | (2H, s), 5.87 (1H, t, J = 5.9 Hz), 6.47 (2H, s), 6.63 (1H, d, J = 7.6 Hz), 7.06 (1H, d, J = 5.6 Hz), |
| | 7.19 (1H, t, J = 8.0 Hz), 7.32 (1H, d, J = 8.3 Hz), 7.70 (1H, d, J = 6.1 Hz), 7.76 (2H, s) |
| 6648 | 1.47 (2H, dd, J = 4.5, 1.6 Hz), 1.63 (2H, dd, J = 4.4, 1.5 Hz), 3.50 (2H, d, J = 6.0 Hz), 3.53 (2H, |
| | s), 4.75 (2H, s), 6.02 (1H, t, J = 5.8 Hz), 6.71 (1H, d, J = 7.7 Hz), 6.91 (2H, s), 7.14 (1H, d, J = |
| | 6.3 Hz), 7.25 (1H, t, J = 8.0 Hz), 7.38 (1H, d, J = 8.3 Hz), 7.68 (1H, d, J = 5.9 Hz), 7.70 (1H, d, J = |
| | 0.9 Hz), 8.07 (1H, d, J = 0.9 Hz) |
| 6649 | 1.50 (2H, dd, J = 4.4, 1.6 Hz), 1.69 (2H, dd, J = 4.4, 1.5 Hz), 1.84-1.93 (2H, m), 2.18 (2H, t, J = |
| | 8.0 Hz), 3.33 (2H, s), 3.47 (2H, s), 3.50 (2H, d, J = 5.9 Hz), 3.52 (2H, s), 5.96 (1H, t, J = 5.9 |
| | Hz), 6.73 (1H, d, J = 7.7 Hz), 6.84 (2H, s), 7.15 (1H, d, J = 6.4 Hz), 7.25 (1H, t, J = 8.0 Hz), 7.38 |
| | (1H, d, J = 8.3 Hz), 7.69 (1H, d, J = 6.3 Hz) |
| 6650 | 1.39 (6H, s), 1.58 (2H, d, J = 3.3 Hz), 1.85 (2H, d, J = 4.4 Hz), 3.55 (2H, d, J = 5.8 Hz), 3.73 |
| | (2H, s), 4.34 (2H, s), 5.94 (1H, t, J = 6.1 Hz), 6.50 (2H, s), 6.69 (1H, d, J = 7.7 Hz), 6.95 (1H, |
| | dd, J = 8.4, 2.1 Hz), 7.10 (1H, d, J = 6.1 Hz), 7.19 (1H, d, J = 2.2 Hz), 7.22 (1H, d, J = 7.9 Hz), |
| | 7.33 (1H, d, J = 8.3 Hz), 7.46 (1H, d, J = 8.4 Hz), 7.72 (1H, d, J = 6.1 Hz), 8.39 (1H, s) |
| 6651 | 1.46-1.56 (2H, m), 1.76-1.80 (2H, m), 2.80 (2H, s), 3.70 (2H, s), 4.42 (2H, s), 5.21 (2H, s), |
| | 6.75 (1H, s), 7.14 (1H, dd, J = 8.4, 2.3 Hz), 7.26 (1H, d, J = 2.2 Hz), 7.71-7.79 (2H, m), 8.19 |
| | (1H, d, J = 5.4 Hz) |
| 6653 | 1.58 (2H, dd, J = 4.5, 1.7 Hz), 1.81-1.87 (2H, m), 1.97-2.09 (2H, m), 2.45-2.49 (2H, m), |
| | 3.54 (2H, d, J = 5.9 Hz), 3.71 (2H, s), 3.79 (2H, t, J = 7.0 Hz), 4.24 (2H, s), 5.89 (1H, t, J = 6.0 |
| | Hz), 6.48 (2H, s), 6.66-6.74 (2H, m), 7.10 (1H, d, J = 5.9 Hz), 7.16 (1H, ddd, J = 8.2, 2.0, 1.0 |
| | Hz), 7.18-7.26 (2H, m), 7.30-7.35 (2H, m), 7.72 (1H, d, J = 6.1 Hz) |
| 6654 | 1.54 (2H, dd, J = 4.5, 1.7 Hz), 1.75-1.87 (4H, m), 2.12 (2H, t, J = 8.0 Hz), 3.51 (2H, t, J = 6.9 |
| | Hz), 3.55 (2H, d, J = 6.2 Hz), 3.70 (2H, s), 4.21 (2H, s), 5.97-6.05 (1H, m), 6.48 (2H, s), 6.67 |
| | (1H, d, J = 7.7 Hz), 6.93 (1H, td, J = 7.6, 1.3 Hz), 7.05 (1H, dd, J = 8.4, 1.2 Hz), 7.12 (1H, d, J = |
| | 5.8 Hz), 7.15 (1H, dd, J = 7.7, 1.7 Hz), 7.18-7.28 (2H, m), 7.33 (1H, d, J = 8.4 Hz), 7.72 (1H, |
| | d, J = 6.1 Hz) |
| 6656 | 1.61 (2H, d, J = 4.4 Hz), 1.89 (2H, d, J = 4.5 Hz), 2.14 (3H, d, J = 1.1 Hz), 2.42 (3H, s), 3.41 |
| | (2H, d, J = 6.0 Hz), 3.56 (3H, s), 3.75 (2H, s), 4.33 (2H, s), 5.21 (1H, t, J = 6.0 Hz), 6.45 (1H, d, |
| | J = 2.0 Hz), 6.51 (1H, dd, J = 8.5, 1.9 Hz), 6.71 (1H, d, J = 1.2 Hz), 7.00 (1H, dd, J = 9.8, 2.3 |
| | Hz), 7.15 (1H, d, J = 8.5 Hz), 7.29 (1H, s), 7.43 (1H, d, J = 9.8 Hz), 7.85 (1H, d, J = 2.3 Hz) |
| 6658 | 1.57 (2H, dd, J = 4.5, 1.7 Hz), 1.83 (2H, dd, J = 4.6, 1.7 Hz), 2.42 (3H, s), 3.56 (2H, d, J = 6.1 |
| | Hz), 3.73 (2H, s), 4.32 (2H, s), 5.94 (1H, t, J = 6.1 Hz), 6.63-6.67 (1H, m), 6.87 (1H, d, J = 8.6 |
| | Hz), 6.99 (1H, dd, J = 9.7, 2.3 Hz), 7.29 (1H, s), 7.38-7.47 (3H, m), 7.84 (1H, d, J = 2.3 Hz) |
| 6659 | 1.56 (2H, dd, J = 4.5, 1.7 Hz), 1.80 (2H, dd, J = 4.5, 1.7 Hz), 2.41 (3H, s), 3.45 (2H, d, J = 6.1 |
| | Hz), 3.58 (2H, q, J = 11.4 Hz), 3.73 (2H, s), 4.31 (2H, s), 5.35 (1H, t, J = 6.1 Hz), 6.54-6.59 |
| | (1H, m), 6.68 (1H, dd, J = 8.3, 1.2 Hz), 6.97 (1H, dd, J = 9.7, 2.3 Hz), 7.07 (1H, d, J = 7.2 Hz), |
| | 7.09-7.14 (1H, m), 7.29 (1H, d, J = 1.0 Hz), 7.43 (1H, dd, J = 9.7, 0.8 Hz), 7.83 (1H, d, J = 2.4 Hz) |
| 6660 | 1.57 (2H, dd, J = 4.5, 1.7 Hz), 1.84 (2H, d, J = 4.5 Hz), 2.42 (3H, d, J = 0.9 Hz), 3.73 (2H, s), |
| | 4.33 (2H, s), 5.26 (2H, s), 5.60 (1H, d, J = 2.1 Hz), 5.89 (1H, dd, J = 5.9, 2.1 Hz), 6.06 (1H, t, J = |
| | 6.0 Hz), 7.00 (1H, dd, J = 9.7, 2.4 Hz), 7.29 (1H, d, J = 1.0 Hz), 7.41-7.46 (2H, m), 7.83- |
| | 7.87 (1H, m) |
| 6661 | 1.59 (2H, dd, J = 4.4, 1.7 Hz), 1.86 (2H, dd, J = 4.5, 1.7 Hz), 2.42 (3H, s), 3.39 (2H, d, J = 6.0 |
| | Hz), 3.74 (2H, s), 4.33 (2H, s), 5.87 (1H, t, J = 6.0 Hz), 6.94-7.02 (2H, m), 7.05 (1H, dd, J = |
| | 8.3, 4.5 Hz), 7.29 (1H, s), 7.44 (1H, d, J = 9.7 Hz), 7.74 (1H, dd, J = 4.5, 1.4 Hz), 7.85 (1H, d, J = |
| | 2.4 Hz), 8.03 (1H, d, J = 2.9 Hz) |
| 6663 | 1.50-1.57 (2H, m), 1.78-1.84 (2H, m), 2.42 (3H, s), 3.60 (2H, d, J = 5.8 Hz), 3.69-3.73 (5H, |
| | m), 4.32 (2H, s), 6.08 (1H, d, J = 2.3 Hz), 6.12 (1H, dd, J = 5.8, 2.2 Hz), 6.36 (1H, t, J = 5.9 Hz), |
| | 7.00 (1H, dd, J = 9.7, 2.3 Hz), 7.29 (1H, s), 7.43 (1H, d, J = 9.7 Hz), 7.76 (1H, d, J = 5.8 Hz), |
| | 7.84 (1H, d, J = 2.3 Hz) |
| 7702 | 1.37 (6H, s), 1.95-2.05 (4H, m), 4.27 (2H, s), 4.28 (2H, s), 6.62 (1H, dd, J = 7.4, 2.5 Hz), 6.72 |
| | (2H, s), 7.04 (1H, d, J = 2.5 Hz), 7.08-7.12 (1H, m), 7.12-7.15 (1H, m), 7.35 (1H, t, J = 8.1 |
| | Hz), 7.39 (1H, d, J = 1.3 Hz), 7.73 (1H, d, J = 8.4 Hz), 7.75 (1H, d, J = 1.1 Hz), 7.81 (1H, d, J = |
| | 5.9 Hz), 8.33-8.44 (1H, m) |
| 7703 | 1.33 (6H, s), 1.82-1.89 (4H, m), 3.47 (2H, d, J = 5.7 Hz), 4.10 (2H, s), 5.90 (1H, t, J = 6.0 Hz), |
| | 6.51 (2H, s), 6.67 (1H, d, J = 7.7 Hz), 6.71 (1H, s), 7.00 (1H, dd, J = 9.7, 2.3 Hz), 7.21 (1H, t, J = |
| | 8.0 Hz), 7.33 (1H, d, J = 8.4 Hz), 7.43 (1H, d, J = 9.7 Hz), 7.48 (1H, d, J = 1.1 Hz) 7.73 (1H, t, |
| | J = 6.2 Hz), 7.79-7.83 (1H, m), 8.29 (1H, d, J = 2.0 Hz) |
| 7704 | 1.37 (6H, s), 1.99-2.04 (4H, m), 4.19 (2H, s), 4.29 (2H, s), 6.71 (2H, s), 7.05 (1H, dd, J = 9.7, |
| | 2.2 Hz), 7.11 (2H, d, J = 6.6 Hz), 7.35 (1H, t, J = 8.1 Hz), 7.48 (1H, d, J = 9.8 Hz), 7.51 (1H, s), |
| | 7.73 (1H, t, J = 8.3 Hz), 7.81 (1H, d, J = 5.9 Hz), 7.83 (1H, s), 8.35 (1H, d, J = 1.8 Hz) |
| 8801 | 2.12-2.23 (2H, m), 2.52-2.59 (2H, m), 3.97-4.07 (5H, m), 4.43 (2H, d, J = 1.0 Hz), 6.57 |
| | (1H, d, J = 7.6 Hz), 6.61 (2H, s), 6.82-6.88 (2H, m), 7.26 (1H, t, J = 8.0 Hz), 7.47-7.49 (1H, |
| | m), 7.51-7.57 (2H, m), 7.67 (1H, d, J = 6.1 Hz), 7.92 (1H, t, J = 1.0 Hz), 8.51 (1H, dd, J = 6.9, |
| | 1.0 Hz) |
| 8803 | 1.90-2.06 (4H, m), 2.26-2.38 (2H, m), 3.25-3.37 (4H, m), 4.03-4.19 (1H, m), 4.42 (2H, d, |
| | J = 6.0 Hz), 6.55 (2H, d, J = 5.3 Hz), 6.80-6.86 (1H, m), 6.95 (1H, dd, J = 16.7, 7.7 Hz), 7.05 |
| | (1H, d, J = 6.1 Hz), 7.27 (1H, td, J = 8.0, 3.9 Hz), 7.47 (1H, s), 7.53 (1H, d, J = 0.9 Hz), 7.62 |
| | (1H, dd, J = 11.7, 8.2 Hz), 7.70 (1H, dd, J = 6.1, 4.2 Hz), 7.90 (1H, s), 8.49 (1H, dd, J = 6.9, 3.0 Hz) |
| 8804 | 1.96-2.05 (4H, m), 2.31-2.38 (2H, m), 3.25-3.34 (4H, m), 4.09 (1H, p, J = 7.0 Hz), 4.42 |
| | (2H, s), 6.56 (2H, s), 6.84 (1H, dd, J = 7.0, 1.6 Hz), 6.97 (1H, d, J = 7.6 Hz), 7.05 (1H, d, J = 6.2 |
| | Hz), 7.27 (1H, t, J = 8.0 Hz), 7.47 (1H, s), 7.53 (1H, d, J = 1.2 Hz), 7.63 (1H, d, J = 8.2 Hz), 7.70 |
| | (1H, d, J = 6.1 Hz), 7.90 (1H, s), 8.49 (1H, dd, J = 6.9, 1.0 Hz). Signals at 3.3 ppm overlap with |
| | water peak. |
| 8805 | 1.95 (2H, t, J = 6.6 Hz), 1.98-2.05 (2H, m), 2.26-2.34 (2H, m), 3.28-3.38 (4H, m), 4.13 |
| | (1H, p, J = 7.0 Hz), 4.43 (2H, s), 6.55 (2H, s), 6.83 (1H, dd, J = 7.0, 1.7 Hz), 6.94 (1H, d, J = 7.7 |
| | Hz), 7.05 (1H, d, J = 6.2 Hz), 7.26 (1H, t, J = 8.0 Hz), 7.47 (1H, s), 7.53 (1H, d, J = 1.2 Hz), 7.61 |
| | (1H, d, J = 8.3 Hz), 7.69 (1H, d, J = 6.1 Hz), 7.90 (1H, s), 8.49 (1H, d, J = 6.8 Hz). Signals at 3.3 |
| | ppm overlap with water peak. |
| 8806 | (CD₃OD) 2.09 (4H, td, J = 7.4, 3.9 Hz), 2.29-2.37 (2H, m), 2.82 (1H, tt, J = 8.5, 6.3 Hz), 3.42 |
| | (2H, t, J= 6.8 Hz), 3.51 (2H, s), 4.09 (2H, d, J = 6.4 Hz), 6.66 (1H, dd, J = 7.4, 2.4 Hz), 6.88 |
| | (1H, d, J = 2.5 Hz), 7.15 (1H, dd, J = 7.8,0.9 Hz), 7.31 (1H, dd, J = 6.3, 1.0 Hz), 7.36-7.44 (2H, |
| | m), 7.62 (1H, dt, J = 8.4, 1.0 Hz), 7.66 (1H, dd, J = 1.5, 0.7 Hz), 7.71(1H, d, J = 6.3 Hz), 8.28 |
| | (1H, dd, J = 7.4, 0.7 Hz) |
| 8807 | (CD₃OD) 2.05-2.14 (2H, m), 2.18 (2H, t, J = 6.6 Hz), 2.22-2.30 (2H, m), 2.87 (1H, tt, J = 8.3, |
| | 6.1 Hz),3.43 (2H, s), 3.48 (2H, q, J = 7.2 Hz), 4.05 (2H, d, J = 6.2 Hz), 6.59-6.69 (1H, m), 6.85 |
| | (1H, d, J = 2.5 Hz), 7.11 (1H, dd, J =7.8, 0.9 Hz), 7.29 (1H, dd, J = 6.4, 1.0 Hz), 7.34-7.49 (2H, |
| | m), 7.59 (1H, dt, J = 8.4, 1.0 Hz), 7.61-7.73 (2H, m), 8.22-8.31 (1H, m) |
| 8808 | 2.07-2.18 (2H, m), 2.37-2.47 (2H, m), 2.59-2.73 (1H, m), 3.95 (2H, d, J = 6.5 Hz), 4.07 |
| | (4H, d, J = 23.8 Hz), 6.56 (2H, s), 6.59 (1H, d, J = 7.6 Hz), 6.89 (1H, dd, J = 6.2, 0.9 Hz), 7.03 |
| | (1H, dd, J = 9.7, 2.4 Hz), 7.27 (1H, t, J = 7.9 Hz), 7.47 (1H, d, J = 9.7 Hz), 7.50 (1H, d, J = 1.1 |
| | Hz), 7.54 (1H, d, J = 8.3 Hz), 7.68 (1H, d, J = 6.1 Hz), 7.83 (1H, t, J = 0.9 Hz), 8.28 (1H, dd, J = |
| | 2.4, 0.8 Hz) |
| 9001 | 1.66-1.81 (2H, m), 1.90-1.99 (1H, m), 2.07-2.15 (1H, m), 3.94 (2H, dd, J = 15.2, 7.7 Hz), |
| | 4.00-4.09 (2H, m), 4.49 (1H, d, J = 7.3 Hz), 4.56-4.68 (2H, m), 6.57 (2H, s), 6.62 (1H, d, J = |
| | 7.7 Hz), 6.83 (1H, dd, J = 7.0, 1.7 Hz), 6.87 (1H, d, J = 6.1 Hz), 7.27 (1H, t, J = 7.9 Hz), 7.48 |
| | (1H, d, J = 1.6 Hz), 7.52 (1H, d, J = 1.2 Hz), 7.54 (1H, d, J = 8.3 Hz), 7.66 (1H, d, J = 6.1 Hz), |
| | 7.87 (1H, t, J = 1.0 Hz), 8.43 (1H, dd, J = 7.0, 0.9 Hz) |
| 9002 | 1.68-1.81 (2H, m), 1.90-1.99 (1H, m), 2.06-2.16 (1H, m), 3.88-4.00 (2H, m), 4.00-4.12 |
| | (2H, m), 4.49 (1H, d, J = 7.4 Hz), 4.55-4.71 (2H, m), 6.56 (2H, s), 6.61 (1H, d, J = 7.7 Hz), |
| | 6.83 (1H, dd, J = 7.0, 1.6 Hz), 6.87 (1H, d, J = 6.1 Hz), 7.27 (1H, t, J = 7.9 Hz), 7.48 (1H, s), |
| | 7.50-7.57 (2H, m), 7.66 (1H, d, J = 6.0 Hz), 7.87 (1H, s), 8.43 (1H, d, J = 7.3 Hz). |
| 9003 | 1.67-1.82 (2H, m), 1.90-1.98 (1H, m), 2.07-2.15 (1H, m), 3.88-3.98 (2H, m), 4.00-4.09 |
| | (2H, m), 4.49 (1H, d, J = 7.4 Hz), 4.56-4.68 (2H, m), 6.56 (2H, s), 6.61 (1H, d, J = 7.6 Hz), |
| | 6.83 (1H, dd, J = 7.0, 1.7 Hz), 6.87 (1H, d, J = 6.1 Hz), 7.27 (1H, t, J = 8.0 Hz), 7.48 (1H, s), |
| | 7.50-7.57 (2H, m), 7.66 (1H, d, J = 6.1 Hz), 7.87 (1H, s), 8.43 (1H, d, J = 6.9 Hz). |
| 10901 | 2.03 (2H, dd, J = 8.5, 6.1 Hz), 2.07-2.12 (2H, m), 2.15 (2H, t, J = 6.9 Hz), 3.32 (2H, s), 3.86 |
| | (2H, s), 4.26 (2H, t, J = 6.9 Hz), 4.65 (1H, s), 5.40 (2H, s), 6.51 (1H, dd, J = 7.4, 2.5 Hz), 6.74 |
| | (1H, d, J = 7.8 Hz), 6.84 (1H, d, J = 2.4 Hz), 7.00 (1H, dd, J = 6.3, 1.0 Hz), 7.14 (1H, d, J = 8.3 |
| | Hz), 7.36 (1H, t, J = 8.0 Hz), 7.41 (1H, d, J = 1.2 Hz), 7.48 (1H, d, J = 1.4 Hz), 7.86-7.97 (2H, m) |
| 10903 | 1.66-1.73 (2H, m), 1.80 (2H, t, J = 8.5 Hz), 1.87 (2H, t, J = 6.9 Hz), 1.94-2.01 (2H, m), 2.69 |
| | (1H, dd, J = 16.6, 5.3 Hz), 2.92 (1H, dd, J = 16.6, 4.5 Hz), 3.18 (2H, d, J = 5.8 Hz), 3.30 (2H, d, |
| | J = 2.0 Hz), 3.79-3.88 (3H, m), 4.02 (2H, t, J = 6.8 Hz), 5.68 (1H, t, J = 6.0 Hz), 6.50 (2H, s), |
| | 6.60 (1H, d, J = 7.7 Hz), 6.76 (1H, d, J = 1.2 Hz), 6.92 (1H, d, J = 1.2 Hz), 7.07 (1H, d, J = 6.1 |
| | Hz), 7.19 (1H, t, J = 8.0 Hz), 7.32 (1H, d, J = 8.3 Hz), 7.72 (1H, d, J = 6.0 Hz) |
| 10904 | 1.82-1.90 (2H, m), 1.97 (2H, d, J = 8.0 Hz), 2.04 (2H, t, J = 6.8 Hz), 3.24 (2H, d, J = 5.8 Hz), |
| | 3.79 (2H, s), 4.10 (2H, t, J = 6.8 Hz), 5.75 (1H, t, J = 6.0 Hz), 6.50 (2H, s), 6.65 (1H, d, J = 7.7 |
| | Hz), 6.94 (1H, dd, J = 9.7, 2.4 Hz), 7.09-7.14 (1H, m), 7.21 (1H, t, J = 8.0 Hz), 7.33 (1H, d, J = |
| | 8.3 Hz), 7.42 (1H, dd, J = 9.7, 0.9 Hz), 7.47 (1H, d, J = 1.1 Hz), 7.73 (1H, d, J = 6.1 Hz), 7.77 |
| | (1H, t, J = 0.9 Hz), 8.22 (1H, dd, J = 2.4, 0.8 Hz) |
| 10906 | 1.81 (2H, td, J = 6.7, 1.5 Hz), 1.88-1.95 (2H, m), 1.95-2.01 (2H, m), 3.22 (2H, d, J = 5.6 Hz), |
| | 3.30 (3H, s, in water peak), 3.77 (2H, s), 4.07 (2H, t, J = 6.8 Hz), 5.74 (1H, d, J = 2.7 Hz), 5.74- |
| | 5.79 (1H, m), 5.83 (1H, dd, J = 7.5, 2.7 Hz), 6.59-6.70 (3H, m), 7.09-7.14 (1H, m), 7.22 |
| | (1H, t, J = 8.0 Hz), 7.35 (1H, d, J = 8.3 Hz), 7.51 (1H, d, J = 7.5 Hz), 7.72 (1H, d, J = 6.2 Hz) |
| 10907 | 1.80 (2H, dd, J = 7.0, 2.5 Hz), 1.95 (2H, d, J = 7.8 Hz), 2.02 (2H, t, J = 6.8 Hz), 2.30 (3H, d, J = |
| | 1.0 Hz), 3.25 (2H, d, J = 6.2 Hz), 3.86 (2H, s), 4.07 (2H, t, J = 6.8 Hz), 6.09 (1H, d, J = 5.6 Hz), |
| | 6.14 (1H, t, J = 6.2 Hz), 6.25 (1H, dd, J = 2.1, 1.1 Hz), 6.51 (1H, dd, J = 7.4, 2.5 Hz), 6.87 (1H, |
| | d, J = 2.5 Hz), 7.35 (1H, d, J = 1.3 Hz), 7.68 (1H, d, J = 5.5 Hz), 7.71 (1H, dd, J = 1.3, 0.7 Hz), |
| | 8.34 (1H, dd, J = 7.4, 0.7 Hz), 10.91 (1H, s) |
| 10908 | 1.68-1.90 (7H, m), 1.96-2.03 (1H, m), 2.58-2.72 (2H, m), 3.18 (2H, d, J = 5.7 Hz), 3.28- |
| | 3.36 (2H, m), 3.82-3.88 (2H, m), 3.97-4.03 (3H, m), 5.69 (1H, t, J = 5.9 Hz), 6.50 (2H, s), |
| | 6.60 (1H, d, J = 7.7 Hz), 6.76 (1H, d, J = 0.9 Hz), 6.88 (1H, d, J = 1.2 Hz), 7.07 (1H, d, J = 6.1 |
| | Hz), 7.19 (1H, t, J = 8.0 Hz), 7.32 (1H, d, J = 8.3 Hz), 7.73 (1H, d, J = 6.1 Hz) |
| 10909 | 1H NMR (CD3CN) δ 1.82 (2H, dd, J = 7.2, 2.4 Hz), 1.89-1.92 (2H, m), 2.00 (2H, t, J = 6.8 Hz), |
| | 3.26 (2H, d, J = 6.0 Hz), 3.71 (2H, s), 4.05 (2H, t, J = 6.8 Hz), 4.99 (1H, s), 5.16 (1H, t, J = 5.8 |
| | Hz), 6.19 (1H, t, J = 6.0 Hz), 6.35 (1H, s), 6.86 (1H, dd, J = 9.8, 2.3 Hz), 7.31 (1H, d, J = 9.7 |
| | Hz), 7.38 (1H, d, J = 0.8 Hz), 7.55 (1H, s), 7.77 (1H, J = 5.7 Hz), 7.82 (1H, d, J = 2.0 Hz) |
| 10910 | 1.84-1.91 (2H, m), 1.98 (2H, t, J = 7.2 Hz), 2.05 (2H, t, J = 6.8 Hz), 3.24 (2H, d, J = 5.7 Hz), |
| | 4.09 (2H, t, J = 6.8 Hz), 4.21 (2H, s), 5.72 (1H, t, J = 5.9 Hz), 6.52 (2H, s), 6.64 (1H, d, J = 7.7 |
| | Hz), 7.08-7.11 (1H, m), 7.13 (1H, d, J = 9.9 Hz), 7.20 (1H, t, J = 8.0 Hz), 7.33 (1H, d, J = 8.3 |
| | Hz), 7.57 (1H, t, J = 51.5 Hz), 7.73 (1H, d, J = 6.0 Hz), 8.35 (1H, d, J = 9.9 Hz) |
| 10911 | 1.78-1.87 (2H, m), 1.88-1.96 (2H, m), 2.01 (2H, t, J = 6.9 Hz), 3.22 (2H, d, J = 5.7 Hz), 3.40 |
| | (3H, s), 3.72 (2H, s), 4.08 (2H, t, J = 6.8 Hz), 5.72 (1H, t, J = 5.9 Hz), 6.04 (1H, t, J = 7.1 Hz), |
| | 6.51 (2H, s), 6.63 (1H, d, J = 7.7 Hz), 6.78 (1H, dd, J = 7.5, 1.8 Hz), 7.10 (1H, d, J = 6.2 Hz), |
| | 7.20 (1H, t, J = 8.0 Hz), 7.24 (1H, dd, J = 6.9, 1.7 Hz), 7.33 (1H, d, J = 8.3 Hz), 7.72 (1H, d, J = |
| | 6.1 Hz) |
| 10912 | 1.81-1.86 (2H, m), 1.90-1.97 (2H, m), 2.01 (2H, t, J = 6.8 Hz), 3.23 (2H, d, J = 5.7 Hz), 4.08 |
| | (2H, t, J = 6.6 Hz), 4.16 (2H, s), 5.71 (1H, t, J = 5.8 Hz), 6.50 (2H, s), 6.63 (1H, d, J = 7.7 Hz), |
| | 7.08-7.11 (2H, m), 7.20 (1H, t, J = 8.0 Hz), 7.33 (1H, d, J = 8.2 Hz), 7.72 (1H, d, J = 6.1 Hz), |
| | 8.55 (2H, d, J = 4.6 Hz) |
| 10914 | 1.78-1.85 (2H, m), 1.88-1.97 (2H, m), 1.97-2.04 (2H, m), 3.23 (2H, d, J = 5.8 Hz), 4.08 |
| | (2H, t, J = 6.8 Hz), 4.11 (2H, s), 5.72 (1H, t, J = 6.0 Hz), 6.61 (2H, s), 6.66 (1H, d, J = 7.8 Hz), |
| | 6.72 (1H, dd, J = 8.3, 1.0 Hz), 6.93 (1H, ddd, J = 7.1, 5.0, 1.0 Hz), 7.11 (1H, d, J = 6.1 Hz), 7.22 |
| | (1H, t, J = 8.0 Hz), 7.35 (1H, d, J = 8.3 Hz), 7.61-7.69 (1H, m), 7.72 (1H, d, J = 6.1 Hz), 8.10 |
| | (1H, ddd, J = 5.0, 2.0, 0.8 Hz) |
| 10915 | 1.73-1.82 (4H, m), 1.84 (2H, t, J = 6.9 Hz), 3.14 (2H, d, J = 5.8 Hz), 3.89 (2H, s), 4.00 (2H, t, J = |
| | 6.8 Hz), 5.61 (1H, t, J = 5.9 Hz), 6.11 (1H, td, J = 6.6, 1.4 Hz), 6.33 (1H, dd, J = 9.3, 1.3 Hz), |
| | 6.49 (2H, s), 6.59 (1H, d, J = 7.7 Hz), 7.03 (1H, d, J = 6.1 Hz), 7.18 (1H, t, J = 8.0 Hz), 7.33 (2H, |
| | ddt, J = 8.9, 4.0, 2.1 Hz), 7.46 (1H, dd, J = 6.8, 2.1 Hz), 7.71 (1H, d, J = 6.1 Hz) |
| 10918 | (CDCl₃) 1.90-2.04 (4H, m), 2.11 (2H, t, J = 6.9 Hz), 3.15 (2H, s), 3.86 (2H, s), 3.97 (1H, s), |
| | 4.16-4.22 (2H, m), 6.56 (3H, ddd, J = 12.8, 8.1, 1.7 Hz), 6.93 (1H, d, J = 2.4 Hz), 7.07-7.14 |
| | (2H, m), 7.42 (1H, s), 7.50 (1H, s), 7.96 (1H, d, J = 7.4 Hz) |
| 10919 | (CDCl₃) 1.91-2.05 (4H, m), 2.11 (2H, t, J = 6.9 Hz), 3.17 (2H, d, J = 5.0 Hz), 3.88 (2H, s), 4.05 |
| | (1H, d, J = 6.0 Hz), 4.19 (2H, t, J = 6.9 Hz), 6.50 (1H, ddd, J = 8.2, 2.3, 0.9 Hz), 6.58-6.65 (2H, |
| | m), 6.65 (1H, ddd, J = 7.9, 2.0, 0.9 Hz), 7.00 (1H, d, J = 2.4 Hz), 7.05 (1H, t, J = 8.0 Hz), 7.41- |
| | 7.45 (1H, m), 7.51 (1H, d, J = 1.6 Hz), 7.98 (1H, d, J = 7.4 Hz) |
| 10920 | 1.76-1.83 (2H, m), 1.88 (3H, d, J = 1.1 Hz), 1.99-2.14 (4H, m), 3.25 (2H, d, J = 5.9 Hz), 3.83 |
| | (2H, s), 4.11 (2H, t, J = 6.8 Hz), 5.88 (1H, t, J = 6.1 Hz), 6.49 (2H, s), 6.62 (1H, d, J = 7.7 Hz), |
| | 6.80 (1H, s), 7.12 (1H, d, J = 6.1 Hz), 7.19 (1H, t, J = 8.0 Hz), 7.26-7.34 (2H, m), 7.59 (1H, d, |
| | J = 1.2 Hz), 7.73 (1H, d, J = 6.1 Hz), 8.18 (1H, d, J = 1.4 Hz) |
| 10921 | 1.80-1.88 (2H, m), 1.93-2.06 (4H, m), 3.36 (2H, d, J = 6.1 Hz), 3.87 (2H, s), 4.07 (2H, t, J = |
| | 6.8 Hz), 6.48 (1H, dd, J = 7.4, 2.5 Hz), 6.54 (1H, d, J = 5.5 Hz), 6.86 (1H, d, J = 2.5 Hz), 7.28 |
| | (1H, t, J = 6.3 Hz), 7.35 (1H, d, J = 1.3 Hz), 7.45 (1H, dd, J = 9.0, 2.3 Hz), 7.71 (1H, t, J = 1.0 |
| | Hz), 7.78 (1H, d, J = 2.3 Hz), 8.29-8.37 (2H, m), 8.37 (1H, d, J = 5.4 Hz) |
| 10922 | (CD₃OD) 1.99-2.14 (4H, m), 2.15-2.22 (2H, m), 3.41 (2H, s), 4.03 (2H, s), 4.21-4.27 (2H, |
| | m), 4.62 (2H, s), 6.80-6.93 (1H, m), 7.02 (2H, dd, J = 15.1, 2.4 Hz), 7.15-7.26 (1H, m), 7.56- |
| | 7.63 (2H, m), 7.79 (1H, d, J = 1.8 Hz), 8.07 (1H, d, J = 9.2 Hz), 8.38-8.44 (1H, m) |
| 10923 | 1.80 (2H, dd, J = 7.1, 2.5 Hz), 2.04 (4H, d, J = 8.1 Hz), 2.13 (3H, s), 3.24 (2H, d, J = 5.7 Hz), |
| | 3.88 (2H, s), 4.11 (2H, t, J = 6.8 Hz), 5.85 (1H, t, J = 6.1 Hz), 6.49 (2H, s), 6.62 (1H, d, J = 7.7 |
| | Hz), 6.82 (1H, d, J = 7.5 Hz), 7.12 (1H, d, J = 6.1 Hz), 7.19 (1H, t, J = 8.0 Hz), 7.32 (1H, d, J = |
| | 8.3 Hz), 7.38 (1H, d, J = 1.3 Hz), 7.69-7.75 (2H, m), 8.32 (1H, d, J = 7.4 Hz) |
| 10924 | 1.79-1.87 (2H, m), 1.96 (2H, d, J = 8.1 Hz), 2.04 (2H, t, J = 6.9 Hz), 3.24 (2H, d, J = 5.8 Hz), |
| | 3.81 (2H, s), 4.10 (2H, t, J = 6.8 Hz), 5.75 (1H, t, J = 6.0 Hz), 6.51 (2H, s), 6.64 (1H, d, J = 7.7 |
| | Hz), 7.11 (1H, d, J = 6.1 Hz), 7.22 (1H, s), 7.24 (1H, d, J = 2.1 Hz), 7.33 (1H, d, J = 8.3 Hz), |
| | 7.54 (1H, d, J = 1.1 Hz), 7.73 (1H, d, J = 6.0 Hz), 7.89 (1H, d, J = 1.1 Hz), 8.28 (1H, d, J = 2.1 Hz) |
| 10925 | 1.83 (2H, dd, J = 7.0, 2.5 Hz), 1.97 (2H, dt, J = 8.5, 4.2 Hz), 2.03 (2H, t, J = 6.9 Hz), 2.50 (3H, |
| | d, J = 1.6 Hz), 3.24 (2H, d, J = 5.8 Hz), 3.86 (2H, s), 4.10 (2H, t, J = 6.8 Hz), 5.75 (1H, t, J = 6.0 |
| | Hz), 6.41 (1H, dd, J = 2.5, 1.1 Hz), 6.50 (2H, s), 6.65 (1H, d, J = 7.7 Hz), 6.79 (1H, d, J = 2.4 |
| | Hz), 7.11 (1H, d, J = 6.1 Hz), 7.16-7.26 (1H, m), 7.33 (1H, d, J = 8.3 Hz), 7.42 (1H, d, J = 1.3 |
| | Hz), 7.56-7.64 (1H, m), 7.74 (1H, d, J = 6.1 Hz) |
| 10926 | 1.85 (2H, dd, J = 7.1, 2.1 Hz), 1.97 (2H, d, J = 8.8 Hz), 2.04 (2H, t, J = 6.8 Hz), 3.24 (2H, d, J = |
| | 5.7 Hz), 3.93 (2H, s), 4.10 (2H, t, J = 6.8 Hz), 5.78 (1H, t, J = 5.8 Hz), 6.52 (2H, s), 6.59 (1H, dd, |
| | J = 7.4, 2.2 Hz), 6.64 (1H, d, J = 7.7 Hz), 7.02 (1H, d, J = 2.2 Hz), 7.11 (1H, d, J = 6.1 Hz), |
| | 7.20 (1H, t, J = 8.0 Hz), 7.33 (1H, d, J = 8.3 Hz), 7.73 (1H, d, J = 6.1 Hz), 8.38 (1H, d, J = 7.5 Hz), |
| | 8.45 (1H, s) |
| 10928 | 1.75-1.86 (2H, m), 1.91 (2H, dt, J = 8.4, 4.2 Hz), 1.96 (2H, t, J = 6.9 Hz), 2.25 (3H, s), 3.22 |
| | (2H, d, J = 5.9 Hz), 3.29 (3H, s), 3.74 (2H, s), 4.06 (2H, t, J = 6.8 Hz), 5.64 (1H, d, J = 2.8 Hz), |
| | 5.73-5.81 (2H, m), 6.64-6.71 (3H, m), 7.13 (1H, d, J = 6.3 Hz), 7.23 (1H, t, J = 8.0 Hz), 7.35 |
| | (1H, d, J = 8.3 Hz), 7.71 (1H, d, J = 6.2 Hz) |
| 10929 | 1.81-1.90 (2H, m), 1.96 (2H, dt, J = 8.3, 4.1 Hz), 2.04 (2H, t, J = 6.8 Hz), 3.25 (2H, d, J = 5.8 |
| | Hz), 3.81 (2H, s), 4.10 (2H, t, J = 6.8 Hz), 5.82 (1H, t, J = 6.0 Hz), 6.68 (3H, d, J = 8.8 Hz), 7.09 |
| | (1H, dd, J = 9.9, 2.2 Hz), 7.14 (1H, d, J = 6.3 Hz), 7.23 (1H, t, J = 8.0 Hz), 7.36 (1H, d, J = 8.3 |
| | Hz), 7.67 (1H, d, J = 9.8 Hz), 7.72 (1H, d, J = 6.2 Hz), 8.20 (1H, d, J = 2.2 Hz), 9.05 (1H, s) |
| 10930 | 1.84 (2H, dd, J = 7.1, 2.2 Hz), 1.98 (2H, d, J = 8.8 Hz), 2.04 (2H, t, J = 6.9 Hz), 2.37 (3H, s), |
| | 3.24 (2H, d, J = 4.8 Hz), 3.87 (2H, s), 4.10 (2H, t, J = 6.8 Hz), 5.76 (1H,s), 6.54 (1H, dd, J = 7.5, |
| | 2.4 Hz), 6.57 (2H, s), 6.65 (1H, d, J = 7.7 Hz), 6.84 (1H, d, J = 2.3 Hz), 7.11 (1H, d, J = 6.3 Hz), |
| | 7.13 (1H, s), 7.21 (1H, t, J = 8.0 Hz), 7.34 (1H, d, J = 8.3 Hz), 7.73 (1H, d, J = 6.1 Hz), 8.06 |
| | (1H, d, J = 7.4 Hz), 8.19 (2H, s) |
| 10931 | 1.78-1.88 (2H, m), 1.93-2.00 (2H, m), 2.03 (2H, t, J = 6.9 Hz), 3.24 (2H, d, J = 5.8 Hz), 3.87 |
| | (2H, s), 4.09 (2H, t, J = 6.8 Hz), 4.26 (2H, s), 5.76 (1H, t, J = 6.0 Hz), 6.52 (2H, s), 6.64 (1H, d, |
| | J = 7.7 Hz), 6.94 (1H, dd, J = 8.4, 2.2 Hz), 7.03 (1H, d, J = 2.2 Hz), 7.11 (1H, d, J = 6.1 Hz), 7.20 |
| | (1H, t, J = 8.0 Hz), 7.33 (1H, d, J = 8.3 Hz), 7.51 (1H, d, J = 8.4 Hz), 7.73 (1H, d, J = 6.1 Hz), |
| | 8.30 (1H, s) |
| 10932 | (CD₃CN) 1.92-2.08 (8H, m), 2.12 (2H, t, J = 6.9 Hz), 4.17 (2H, dt, J = 16.4, 6.9 Hz), 4.91 (1H, |
| | t, J = 5.9 Hz), 5.56 (2H, s), 6.62 (1H, dd, J = 7.5, 2.5 Hz), 6.76 (1H, d, J = 7.7 Hz), 6.87 (1H, d, J = |
| | 2.5 Hz), 7.04 (1H, dd, J = 6.2, 1.0 Hz), 7.17-7.25 (1H, m), 7.34 (1H, t, J = 8.0 Hz), 7.84 (1H, |
| | dd, J = 6.1, 3.7 Hz), 7.97 (1H, t, J = 1.1 Hz), 8.15 (1H, d, J = 7.5 Hz) |
| 10933 | 1.76 (2H, dd, J = 7.1, 2.6 Hz), 1.98 (2H, t, J = 6.9 Hz), 2.03 (2H, d, J = 7.8 Hz), 3.22 (2H, d, J = |
| | 5.9 Hz), 3.29 (3H, s), 3.82 (2H, s), 4.08 (2H, t, J = 6.8 Hz), 5.85 (2H, d, J = 8.9 Hz), 6.46 (2H, |
| | s), 6.59 (1H, d, J = 7.8 Hz), 7.10 (1H, d, J = 6.2 Hz), 7.18 (1H, t, J = 8.0 Hz), 7.31 (1H, d, J = 8.3 |
| | Hz), 7.71 (1H, d, J = 6.1 Hz), 7.87 (1H, s) |
| 10934 | (CD₃OD) 8 1.36 (6H, d, J = 6.8 Hz), 1.97-2.08 (4H, m), 2.13 (2H, t, J = 6.9 Hz), 3.35 (2H, s), |
| | 3.87 (2H, s), 4.24 (2H, t, J = 6.9 Hz), 5.11 (1H, hept, J = 6.8 Hz), 5.91 (1H, d, J = 2.8 Hz), 6.12 |
| | (1H, dd, J = 7.8, 2.7 Hz), 6.83 (1H, dd, J = 6.6, 2.2 Hz), 7.11 (1H, dd, J = 6.3, 0.8 Hz), 7.32- |
| | 7.40 (2H, m), 7.56 (1H, d, J = 7.7 Hz), 7.73 (1H, d, J = 6.3 Hz) |
| 10936 | 1.83 (2H, dd, J = 7.1, 2.5 Hz), 1.93 (2H, dt, J = 8.4, 4.2 Hz), 1.99 (2H, t, J = 6.9 Hz), 3.22 (2H, |
| | d, J = 5.8 Hz), 4.04 (2H, s), 4.07 (2H, t, J = 6.8 Hz), 5.69 (1H, t, J = 5.9 Hz), 6.28 (1H, d, J = 1.8 |
| | Hz), 6.49 (2H, s), 6.63 (1H, dd, J = 7.8, 0.8 Hz), 7.09 (1H, dd, J = 6.3, 0.8 Hz), 7.20 (1H, t, J = |
| | 8.0 Hz), 7.33 (1H, d, J = 8.3 Hz), 7.72 (1H, d, J = 6.1 Hz), 8.63 (1H, d, J = 1.8 Hz) |
| 10937 | 1.78-1.86 (2H, m), 1.92 (2H, dt, J = 7.8, 3.9 Hz), 1.99 (2H, t, J = 6.8 Hz), 3.22 (2H, d, J = 5.8 |
| | Hz), 4.07 (2H, t, J = 6.8 Hz), 4.21 (2H, s), 5.69 (1H, t, J = 6.0 Hz), 6.51 (2H, s), 6.63 (1H, d, J = |
| | 7.7 Hz), 6.86 (1H, dd, J = 5.9, 1.2 Hz), 7.08 (1H, d, J = 6.1 Hz), 7.19 (1H, t, J = 8.0 Hz), 7.33 |
| | (1H, d, J = 8.3 Hz), 7.73 (1H, d, J = 6.1 Hz), 8.47 (1H, d, J = 5.8 Hz), 8.72 (1H, d, J = 1.2 Hz) |
| 10938 | 1.82 (2H, dd, J = 7.0, 2.5 Hz), 1.89-1.98 (2H, m), 2.01 (2H, t, J = 6.8 Hz), 2.81 (2H, t, J = 6.5 |
| | Hz), 3.23 (2H, d, J = 5.9 Hz), 3.28-3.32 (2H, m), 3.85 (2H, s), 4.09 (2H, t, J = 6.8 Hz), 5.76 |
| | (1H, t, J = 5.9 Hz), 6.51 (2H, s), 6.64 (1H, d, J = 7.7 Hz), 6.75-6.84 (2H, m), 7.11 (1H, d, J = |
| | 6.1 Hz), 7.20 (1H, t, J = 8.0 Hz), 7.33 (1H, d, J = 8.3 Hz), 7.64-7.79 (3H, m) |
| 10939 | 1.82 (2H, dd, J = 7.1, 2.3 Hz), 1.95 (2H, d, J = 8.8 Hz), 2.02 (2H, t, J = 6.8 Hz), 2.88 (2H, d, J = |
| | 6.6 Hz), 2.97 (3H, s), 3.24 (2H, d, J = 5.3 Hz), 3.48 (2H, t, J = 6.7 Hz), 3.84 (2H, s), 4.09 (2H, t, |
| | J = 6.8 Hz), 5.77 (1H, t, J = 5.6 Hz), 6.58 (2H, s), 6.65 (1H, d, J = 7.8 Hz), 6.75 (1H, d, J = 2.3 |
| | Hz), 6.81 (1H, dd, J = 8.6, 2.5 Hz), 7.12 (1H, d, J = 6.2 Hz), 7.21 (1H, t, J = 8.0 Hz), 7.34 (1H, d, |
| | J = 8.3 Hz), 7.72 (1H, d, J = 2.2 Hz), 7.74 (1H, d, J = 4.8 Hz) |
| 10940 | 1.75-1.83 (2H, m), 1.90 (2H, dt, J = 8.0, 5.6 Hz), 1.96 (2H, t, J = 6.8 Hz), 3.21 (2H, d, J = 5.8 |
| | Hz), 3.30 (3H, s), 4.06 (2H, t, J = 6.8 Hz), 4.11 (2H, s), 5.69 (1H, t, J = 5.9 Hz), 5.85 (1H, d, J = |
| | 7.1 Hz), 6.51 (2H, s), 6.63 (1H, d, J = 7.6 Hz), 7.09 (1H, d, J = 6.1 Hz), 7.20 (1H, t, J = 8.0 Hz), |
| | 7.33 (1H, d, J = 8.3 Hz), 7.72 (1H, d, J = 6.1 Hz), 7.92 (1H, d, J = 7.1 Hz) |
| 10941 | 1.83 (2H, dd, J = 7.1, 2.5 Hz), 1.97 (2H, d, J = 7.7 Hz), 2.03 (2H, t, J = 6.9 Hz), 3.24 (2H, d, J = |
| | 5.8 Hz), 3.88 (2H, s), 4.09 (2H, t, J = 6.8 Hz), 4.26 (2H, s), 5.75 (1H, t, J = 6.0 Hz), 6.55 (2H, s), |
| | 6.65 (1H, d, J = 7.7 Hz), 7.08 (1H, dd, J = 8.3, 2.5 Hz), 7.10-7.14 (2H, m), 7.21 (1H, t, J = 8.0 |
| | Hz), 7.34 (1H, d, J = 8.3 Hz), 7.41 (1H, d, J = 8.3 Hz), 7.73 (1H, d, J = 6.1 Hz), 8.52 (1H, s) |
| 10942 | 1.44 (2H, dd, J = 6.5, 2.5 Hz), 1.58-1.82 (8H, m), 3.10 (2H, d, J = 5.8 Hz), 3.67 (2H, s), 5.81 |
| | (1H, t, J = 6.0 Hz), 6.46 (2H, d, J = 4.7 Hz), 6.58 (1H, d, J = 7.7 Hz), 6.90 (1H, dd, J = 9.7, 2.4 |
| | Hz), 7.14-7.23 (2H, m), 7.29 (1H, d, J = 8.3 Hz), 7.40 (1H, d, J = 9.7 Hz), 7.46 (1H, d, J = 1.1 |
| | Hz), 7.71-7.79 (2H, m), 8.18 (1H, d, J = 2.4 Hz) |
| 10943 | 1.80 (2H, dd, J = 7.1, 2.4 Hz), 1.89 (2H, dt, J = 8.3, 4.1 Hz), 1.99 (2H, t, J = 6.8 Hz), 3.22 (2H, |
| | d, J = 5.8 Hz), 3.33 (3H, s), 3.62 (2H, s), 4.07 (2H, t, J = 6.8 Hz), 5.79 (1H, t, J = 5.9 Hz), 6.29 |
| | (1H, d, J = 9.8 Hz), 6.56 (2H, s), 6.63 (1H, d, J = 7.7 Hz), 7.10 (1H, d, J = 6.1 Hz), 7.16-7.25 |
| | (2H, m), 7.30-7.36 (2H, m), 7.72 (1H, d, J = 6.1 Hz) |
| 10944 | 1.82 (2H, dd, J = 7.1, 2.5 Hz), 1.88-1.96 (2H, m), 2.00 (2H, t, J = 6.9 Hz), 3.22 (2H, d, J = 5.8 |
| | Hz), 3.78 (3H, s), 3.86 (2H, s), 4.08 (2H, t, J = 6.8 Hz), 5.75 (1H, t, J = 6.0 Hz), 6.27 (1H, d, J = |
| | 2.2 Hz), 6.44-6.55 (3H, m), 6.63 (1H, d, J = 7.7 Hz), 7.10 (1H, d, J = 6.2 Hz), 7.20 (1H, t, J = |
| | 8.0 Hz), 7.33 (1H, d, J = 8.3 Hz), 7.72 (1H, d, J = 6.1 Hz), 7.91 (1H, d, J = 5.9 Hz) |
| 10945 | 1.74-1.82 (2H, m), 1.94-2.06 (4H, m), 3.06 (3H, s), 3.25 (2H, d, J = 5.9 Hz), 3.87 (2H, s), |
| | 4.10 (2H, t, J = 6.8 Hz), 5.64 (1H, dd, J = 7.8, 1.1 Hz), 5.93 (1H, dd, J = 9.0, 1.0 Hz), 5.96 (1H, |
| | d, J = 6.1 Hz), 6.60-6.70 (3H, m), 7.11-7.16 (1H, m), 7.21 (1H, t, J = 8.0 Hz), 7.27-7.37 |
| | (2H, m), 7.72 (1H, d, J = 6.1 Hz) |
| 10946 | 1.74-1.84 (2H, m), 1.92-2.03 (4H, m), 3.23 (2H, d, J = 5.9 Hz), 3.59 (3H, s), 3.96 (2H, s), |
| | 4.08 (2H, t, J = 6.8 Hz), 5.79 (1H, t, J = 6.0 Hz), 6.51 (2H, s), 6.63 (1H, d, J = 7.7 Hz), 7.10 (1H, |
| | d, J = 6.1 Hz), 7.16-7.24 (2H, m), 7.33 (1H, d, J = 8.3 Hz), 7.72 (1H, d, J = 6.1 Hz) |
| 10948 | 1.18 (6H, d, J = 6.9 Hz), 1.77-1.84 (2H, m), 1.88-1.96 (2H, m), 1.96-2.03 (2H, m), 2.87 |
| | (1H, h, J = 6.9 Hz), 3.22 (2H, d, J = 5.7 Hz), 4.07 (2H, t, J = 6.8 Hz), 4.18 (2H, s), 5.70 (1H, t, J = |
| | 5.9 Hz), 6.50 (2H, s), 6.63 (1H, d, J = 7.7 Hz), 6.68 (1H, d, J = 1.1 Hz), 7.09 (1H, dd, J = 6.3, |
| | 0.9 Hz), 7.20 (1H, t, J = 8.0 Hz), 7.33 (1H, d, J = 8.3 Hz), 7.73 (1H, d, J = 6.1 Hz), 8.62 (1H, d, J = |
| | 1.1 Hz) |
| 10949 | 1.14 (6H, d, J = 6.7 Hz), 1.80 (2H, dd, J = 7.1, 2.3 Hz), 1.93 (2H, d, J = 8.9 Hz), 1.98 (2H, t, J = |
| | 6.8 Hz), 3.00-3.09 (1H, m), 3.22 (2H, d, J = 5.8 Hz), 3.36 (3H, s), 3.75 (2H, s), 4.07 (2H, d, J = |
| | 6.8 Hz), 5.66 (1H, d, J= 2.6 Hz), 5.69 (1H, d, J= 2.6 Hz), 5.73 (1H, t, J = 5.9 Hz), 6.49 (2H, s), |
| | 6.63 (1H, d, J = 7.8 Hz), 7.10 (1H, d, J = 6.2 Hz), 7.20 (1H, t, J = 8.0 Hz), 7.33 (1H, d, J = 8.2 |
| | Hz), 7.72 (1H, d, J = 6.0 Hz) |
| 10951 | 1.81 (2H, dd, J = 7.1, 2.3 Hz), 1.94 (2H, d, J = 9.0 Hz), 1.99 (2H, t, J = 6.8 Hz), 3.23 (2H, d, J = |
| | 5.8 Hz), 3.40 (3H, s), 3.86 (2H, s), 4.07 (2H, t, J = 6.8 Hz), 5.72 (1H, t, J = 5.9 Hz), 6.09 (1H, d, |
| | J = 2.6 Hz), 6.48 (1H, d, J = 2.2 Hz), 6.49 (2H, s), 6.63 (1H, d, J = 7.7 Hz), 7.09 (1H, d, J = 6.1 |
| | Hz), 7.20 (1H, t, J = 8.0 Hz), 7.33 (1H, d, J = 8.3 Hz), 7.72 (1H, d, J = 6.1 Hz) |
| 10952 | 1.81 (2H, dd, J = 7.1, 2.4 Hz), 1.92 (2H, dt, J = 8.2, 4.1 Hz), 1.99 (2H, t, J = 6.9 Hz), 3.23 (2H, |
| | d, J = 5.8 Hz), 3.77 (2H, s), 4.08 (2H, t, J = 6.8 Hz), 5.64 (1H, d, J = 2.5 Hz), 5.73 (1H, t, J = 6.0 |
| | Hz), 5.77 (1H, dd, J = 7.3, 2.5 Hz), 6.50 (2H, s), 6.64 (1H, d, J = 7.7 Hz), 7.10 (1H, d, J = 6.2 |
| | Hz), 7.16-7.25 (2H, m), 7.33 (1H, d, J = 8.3 Hz), 7.73 (1H, d, J = 6.1 Hz), 11.04 (1H, s) |
| 10953 | 1.77-1.87 (2H, m), 1.88-1.98 (2H, m), 2.01 (2H, t, J = 6.8 Hz), 2.36 (3H, s), 3.23 (2H, d, J = |
| | 5.9 Hz), 3.86 (2H, s), 4.08 (2H, t, J = 6.8 Hz), 5.77 (1H, t, J = 6.0 Hz), 6.53 (2H, s), 6.63 (1H, d, |
| | J = 7.7 Hz), 6.70 (1H, dd, J = 5.8, 2.5 Hz), 6.76 (1H, d, J = 2.5 Hz), 7.11 (1H, d, J = 6.1 Hz), 7.20 |
| | (1H, t, J = 8.0 Hz), 7.33 (1H, d, J = 8.3 Hz), 7.73 (1H, d, J = 6.1 Hz), 8.18 (1H, d, J = 5.8 Hz) |
| 10954 | 1.78-1.83 (2H, m), 1.93-1.99 (2H, m), 2.02 (2H, t, J = 6.8 Hz), 3.22 (2H, d, J = 5.8 Hz), 3.74 |
| | (3H, s), 3.86 (2H, s), 4.09 (2H, t, J = 7.0 Hz), 5.79 (1H, t, J = 5.9 Hz), 6.51 (2H, s), 6.61 (1H, d, |
| | J = 7.7 Hz), 6.98 (1H, d, J = 5.3 Hz), 7.10 (1H, d, J = 6.1 Hz), 7.20 (1H, t, J = 8.0 Hz), 7.33 (1H, |
| | d, J = 8.3 Hz), 7.72 (1H, d, J = 6.1 Hz), 8.07 (1H, d J = 5.3 Hz), 8.11 (1H, s) |
| 10955 | 1.76-1.81 (2H, m), 1.90 (2H, d, J = 8.8 Hz), 1.98 (2H, t, J = 6.8 Hz), 2.22 (3H, s), 3.22 (2H, d, |
| | J = 5.8 Hz), 4.05-4.08 (4H, m), 5.72 (1H, t, J = 5.9 Hz), 6.52 (2H, s), 6.53 (1H, s), 6.63 (1H, d, |
| | J = 7.7 Hz), 6.77 (1H, d, J = 4.7 Hz), 7.09 (1H, d, J = 6.1 Hz), 7.20 (1H, t, J = 8.0 Hz), 7.33 (1H, |
| | d, J = 8.3 Hz), 7.73 (1H, d, J = 6.2 Hz), 7.94 (1H, d, J = 5.2 Hz) |
| 10956 | 1.75-1.84 (2H, m), 1.87-1.95 (2H, m), 1.98 (2H, t, J = 6.8 Hz), 2.17 (3H, s), 3.21 (2H, d, J = |
| | 5.8 Hz), 4.03-4.11 (4H, m), 5.71 (1H, t, J = 6.0 Hz), 6.53 (2H, s), 6.60-6.65 (2H, m), 7.09 |
| | (1H, dd, J = 6.3, 0.8 Hz), 7.19 (1H, t, J = 8.0 Hz), 7.33 (1H, d, J = 8.3 Hz), 7.47 (1H, ddd, J = |
| | 8.4, 2.5, 0.7 Hz), 7.73 (1H, d, J = 6.0 Hz), 7.90 (1H, dt, J = 2.5, 0.9 Hz) |
| 10957 | 1.80 (2H, dd, J = 7.1, 2.3 Hz), 1.93 (2H, d, J = 9.1 Hz), 1.99 (2H, t, J = 6.8 Hz), 2.31 (3H, s), |
| | 3.22 (2H, d, J = 5.8 Hz), 4.07 (4H, t, J = 6.8 Hz), 5.72 (1H, t, J = 5.9 Hz), 6.50 (1H, s), 6.52 (2H, |
| | s), 6.63 (1H, d, J = 7.7 Hz), 6.78 (1H, d, J = 7.1 Hz), 7.09 (1H, d, J = 6.2 Hz), 7.20 (1H, t, J = 8.0 |
| | Hz), 7.33 (1H, d, J = 8.3 Hz), 7.53 (1H, t, J = 7.7 Hz), 7.72 (1H, d, J = 6.0 Hz) |
| 10958 | 1.88 (2H, d, J = 7.5 Hz), 1.98 (2H, d, J = 7.7 Hz), 2.05 (2H, q, J = 6.5 Hz), 3.21 (2H, d, J = 5.6 |
| | Hz), 3.91 (2H, s), 4.09 (2H, t, J = 6.8 Hz), 6.43 (1H, t, J = 5.8 Hz), 6.57 (1H, d, J = 7.6 Hz), 6.66 |
| | (1H, s), 6.89 (1H, s), 7.17 (1H, d, J = 8.8 Hz), 7.34-7.41 (2H, m), 7.72 (2H, t, J = 4.6 Hz), 8.16 |
| | (1H, d, J = 5.9 Hz), 8.36 (1H, d, J = 7.4 Hz), 8.83 (1H, s) |
| 10961 | 1.86 (2H, dd, J = 7.0, 2.5 Hz), 1.93-2.02 (2H, m), 2.05 (2H, t, J = 6.9 Hz), 3.24 (2H, d, J = 5.8 |
| | Hz), 4.00 (2H, s), 4.10 (2H, t, J = 6.8 Hz), 5.75 (1H, t, J = 6.0 Hz), 6.50 (2H, s), 6.64 (1H, d, J = |
| | 7.7 Hz), 6.86 (1H, dd, J = 7.5, 2.4 Hz), 7.11 (1H, d, J = 6.2 Hz), 7.21 (1H, t, J = 8.0 Hz), 7.28- |
| | 7.36 (2H, m), 7.73 (1H, d, J = 6.1 Hz), 8.44 (1H, d, J = 7.5 Hz) |
| 10962 | 1.61 (3H, s), 1.68-1.78 (2H, m), 1.95 (4H, t, J = 7.0 Hz), 3.21 (2H, d, J = 6.0 Hz), 3.25 (3H, s), |
| | 4.03-4.11 (4H, m), 5.83 (1H, t, J = 6.1 Hz), 6.48 (2H, s), 6.60 (1H, d, J = 7.7 Hz), 7.09 (1H, d, |
| | J = 6.1 Hz), 7.18 (1H, t, J = 8.0 Hz), 7.31 (1H, d, J = 8.3 Hz), 7.64-7.76 (2H, m) |
| 10963 | 1.75-1.90 (2H, m), 1.91-2.01 (2H, m), 2.03 (2H, t, J = 6.9 Hz), 3.24 (2H, d, J = 5.8 Hz), 4.00 |
| | (2H, s), 4.09 (2H, t, J = 6.8 Hz), 5.75 (1H, t, J = 6.0 Hz), 6.50 (2H, s), 6.64 (1H, d, J = 7.7 Hz), |
| | 7.10 (1H, d, J = 6.1 Hz), 7.16-7.25 (2H, m), 7.33 (1H, d, J = 8.3 Hz), 7.73 (1H, d, J = 6.1 Hz), |
| | 8.89-8.98 (2H, m) |
| 10964 | 1.09 (3H, t, J = 7.0 Hz), 1.75-1.85 (2H, m), 1.91 (2H, dt, J = 8.4, 4.2 Hz), 1.96 (2H, t, J = 6.9 |
| | Hz), 2.28 (3H, s), 3.22 (2H, d, J = 5.9 Hz), 3.73 (2H, s), 3.87 (2H, q, J = 7.0 Hz), 4.07 (2H, t, J = |
| | 6.8 Hz), 5.62 (1H, d, J = 2.8 Hz), 5.73 (2H, d, J = 2.9 Hz), 6.50 (2H, s), 6.63 (1H, d, J = 7.7 Hz), |
| | 7.10 (1H, d, J = 6.1 Hz), 7.20 (1H, t, J = 8.0 Hz), 7.33 (1H, d, J = 8.3 Hz), 7.73 (1H, d, J = 6.1 Hz) |
| 10965 | 1.73-1.93 (6H, m), 3.18 (2H, d, J = 5.9 Hz), 3.97-4.05 (4H, m), 5.70 (1H, t, J = 6.0 Hz), 6.31 |
| | (1H, dd, J = 7.8, 3.2 Hz), 6.49 (2H, s), 6.60 (1H, d, J = 7.7 Hz), 7.03-7.09 (1H, m), 7.20 (1H, t, |
| | J = 8.0 Hz), 7.33 (1H, d, J = 8.3 Hz), 7.72 (2H, dd, J = 4.6, 1.5 Hz), 8.15 (1H, d, J = 7.8 Hz) |
| 10966 | 1.77-1.87 (4H, m), 1.93 (2H, dt, J = 8.4, 4.2 Hz), 3.19 (2H, d, J = 5.9 Hz), 4.01 (2H, t, J = 6.8 |
| | Hz), 4.28 (2H, s), 5.74 (1H, t, J = 5.9 Hz), 6.46-6.53 (3H, m), 6.60 (1H, d, J = 7.6 Hz), 7.04- |
| | 7.10 (1H, m), 7.20 (1H, t, J = 8.0 Hz), 7.33 (1H, d, J = 8.3 Hz), 7.72 (1H, d, J = 6.1 Hz), 8.16 |
| | (1H, d, J = 6.8 Hz), 8.26 (1H, d, J = 2.7 Hz) |
| 10968 | 1.84 (2H, dd, J = 7.0, 2.5 Hz), 1.95 (2H, dt, J = 8.4, 4.2 Hz), 2.03 (2H, t, J = 6.8 Hz), 3.24 (2H, |
| | d, J = 5.8 Hz), 3.88 (2H, s), 4.09 (2H, t, J = 6.8 Hz), 5.83 (1H, t, J = 6.0 Hz), 6.69 (1H, d, J = 7.8 |
| | Hz), 6.77 (2H, s), 7.15 (1H, d, J = 6.1 Hz), 7.23-7.27 (1H, m), 7.27-7.30 (1H, m), 7.32 (1H, |
| | ddd, J = 8.5, 2.9, 1.5 Hz), 7.37 (1H, d, J = 8.3 Hz), 7.71 (1H, d, J = 6.3 Hz), 8.13 (1H, dd, J = |
| | 4.5, 1.5 Hz), 8.23 (1H, dd, J = 2.8, 0.8 Hz) |
| 10969 | 1.86 (2H, dd, J = 7.1, 2.4 Hz), 1.98 (2H, d, J = 7.8 Hz), 2.05 (2H, t, J = 6.9 Hz), 3.92 (3H, s), |
| | 4.10 (3H, t, J = 6.8 Hz), 5.75 (1H, t, J = 5.9 Hz), 6.30 (1H, s), 6.51 (2H, s), 6.64 (1H, d, J = 7.7 |
| | Hz), 6.75 (2H, dd, J = 7.5, 2.5 Hz), 7.11 (1H, d, J = 6.2 Hz), 7.16-7.26 (2H, m), 7.33 (1H, d, J = |
| | 8.3 Hz), 7.73 (1H, d, J = 6.1 Hz), 7.89 (1H, d, J = 1.0 Hz), 8.38 (1H, s), 8.89 (1H, d, J = 7.6 Hz) |
| 10970 | 1.84 (2H, dd, J = 7.1, 2.6 Hz), 1.97 (2H, d, J = 8.0 Hz), 2.04 (2H, t, J = 6.9 Hz), 3.08 (2H, d, J = |
| | 5.9 Hz), 3.90 (2H, s), 4.09 (2H, t, J = 6.8 Hz), 5.03 (1H, t, J = 6.0 Hz), 6.49 (1H, d, J = 2.2 Hz), |
| | 6.58 (1H, dd, J = 7.4, 2.5 Hz), 6.68 (1H, dd, J = 8.7, 2.2 Hz), 6.89 (1H, d, J = 2.5 Hz), 7.13 (1H, |
| | d, J = 8.7 Hz), 7.27 (1H, s), 7.36 (1H, d, J = 1.3 Hz), 7.72 (1H, d, J = 1.2 Hz), 8.36 (1H, d, J = |
| | 7.3 Hz), 10.86 (1H, s) |
| 10971 | 1.75-1.84 (2H, m), 1.87-1.94 (2H, m), 1.98 (2H, t, J = 6.8 Hz), 2.33 (3H, s), 3.21 (2H, d, J = |
| | 5.8 Hz), 4.07 (2H, t, J = 6.8 Hz), 4.18 (2H, s), 5.70 (1H, t, J = 5.9 Hz), 6.50 (2H, s), 6.62 (1H, d, |
| | J = 7.7 Hz), 6.69 (1H, t, J = 1.0 Hz), 7.06-7.12 (1H, m), 7.19 (1H, t, J = 8.0 Hz), 7.32 (1H, d, J = |
| | 8.3 Hz), 7.73 (1H, d, J = 6.1 Hz), 8.57 (1H, d, J = 1.1 Hz) |
| 10972 | 1.75-1.83 (2H, m), 1.87-2.09 (6H, m), 2.93 (2H, t), 3.22 (2H, d, J = 5.9 Hz), 3.75 (2H, s), |
| | 3.82 (2H, t), 4.07 (2H, t, J = 6.8 Hz), 5.55 (1H, d, J = 2.4 Hz), 5.68-5.80 (2H, m), 6.50 (2H, s), |
| | 6.63 (1H, d, J = 7.7 Hz), 7.10 (1H, d, J = 6.1 Hz), 7.20 (1H, t, J = 8.0 Hz), 7.33 (1H, d, J = 8.3 |
| | Hz), 7.72 (1H, d, J = 6.1 Hz) |
| 10973 | 1.84 (2H, dd, J = 7.1, 2.5 Hz), 1.96 (2H, dt, J = 8.3, 4.2 Hz), 2.04 (2H, t, J = 6.8 Hz), 3.24 (2H, |
| | d, J = 5.7 Hz), 3.87 (2H, s), 4.10 (2H, t, J = 6.8 Hz), 5.75 (1H, t, J = 6.0 Hz), 6.51 (2H, s), 6.64 |
| | (1H, d, J = 7.7 Hz), 6.92 (1H, dd, J = 8.8, 2.4 Hz), 7.10 (1H, d, J = 6.1 Hz), 7.20 (1H, t, J = 8.0 |
| | Hz), 7.30-7.36 (2H, m), 7.61 (1H, d, J = 8.8 Hz), 7.73 (1H, d, J = 6.1 Hz), 8.57 (1H, s) |
| 10974 | 1.77-1.85 (2H, m), 1.90-1.95 (2H, m), 1.99 (2H, t, J = 6.8 Hz), 2.45 (3H, s), 3.22 (2H, d, J = |
| | 5.9 Hz), 4.07 (2H, t, J = 6.8 Hz), 4.17 (2H, s), 5.71 (1H, t, J = 5.9 Hz), 6.50 (2H, s), 6.64 (2H, |
| | m), 7.09 (1H, d, J = 6.1 Hz), 7.20 (1H, t, J = 8.0 Hz), 7.33 (1H, d, J = 8.3 Hz), 7.73 (1H, d, J = |
| | 6.1 Hz), 8.34 (1H, d, J = 5.8 Hz) |
| 10975 | 1.78-1.87 (2H, m), 1.93 (2H, dt, J = 8.3, 4.2 Hz), 1.99 (2H, t, J = 6.9 Hz), 3.23 (2H, d, J = 5.8 |
| | Hz), 3.85 (2H, s), 4.08 (2H, t, J = 6.8 Hz), 5.74 (1H, t, J = 6.0 Hz), 5.84 (1H, d, J = 2.6 Hz), 6.05 |
| | (1H, dd, J = 7.9, 2.6 Hz), 6.51 (2H, s), 6.63 (1H, d, J = 7.6 Hz), 7.10 (1H, d, J = 6.1 Hz), 7.20 |
| | (1H, t, J = 8.0 Hz), 7.33 (1H, d, J = 8.3 Hz), 7.65 (1H, d, J = 7.9 Hz), 7.73 (1H, d, J = 6.1 Hz), |
| | 7.76 (1H, t, J = 60.0 Hz) |
| 10976 | 1.79-1.88 (2H, m), 1.88-2.10 (4H, m), 3.23 (2H, d, J = 5.8 Hz), 4.08 (2H, t, J = 6.8 Hz), 4.17 |
| | (2H, s), 5.69 (1H, t, J = 5.9 Hz), 6.50 (2H, s), 6.64 (1H, d, J = 7.7 Hz), 7.06-7.12 (1H, m), 7.20 |
| | (1H, t, J = 8.0 Hz), 7.33 (1H, d, J = 8.3 Hz), 7.72 (1H, d, J = 6.1 Hz), 8.13-8.20 (2H, m), 8.24 |
| | (1H, d, J = 1.3 Hz) |
| 11001 | 1.77-1.84 (2H, m), 1.87-1.95 (2H, m), 2.02 (2H, t, J = 6.9 Hz), 3.18 (2H, d, J = 5.8 Hz), 3.30 |
| | (3H, s), 3.79 (2H, s), 4.02 (2H, t, J = 6.8 Hz), 5.72 (1H, d, J = 2.8 Hz), 5.85 (1H, dd, J = 7.5, 2.7 |
| | Hz), 5.89 (1H, t, J = 6.0 Hz), 6.45-6.52 (2H, m), 6.61 (1H, d, J = 7.7 Hz), 7.16-7.22 (2H, m), |
| | 7.31 (1H, d, J = 8.3 Hz), 7.51 (1H, d, J = 7.5 Hz), 7.72 (1H, d, J = 6.1 Hz) |
| 12001 | 1.55 (2H, dd, J = 4.5, 1.9 Hz), 1.90 (2H, d, J = 5.6 Hz), 3.30 (3H, s), 3.43 (2H, s), 3.94 (2H, d, J = |
| | 6.2 Hz), 4.13 (2H, s), 5.12 (2H, s), 5.76 (1H, d, J = 2.8 Hz), 5.88 (1H, dd, J = 7.5, 2.8 Hz), |
| | 5.99 (1H, s), 6.53 (2H, s), 6.69 (1H, d, J = 7.7 Hz), 6.91 (1H, d, J = 6.2 Hz), 7.20 (1H, t, J = 8.0 |
| | Hz), 7.25-7.50 (6H, m), 7.52 (1H, d, J = 7.6 Hz), 7.70 (1H, d, J = 6.0 Hz) |
| 12007 | 1.70 (2H, d, J = 4.5 Hz), 1.94 (2H, d, J = 4.9 Hz), 3.25 (2H, s), 3.32 (3H,s), 3.69 (2H, d, J = 4.8 |
| | Hz), 4.21 (2H, s), 5.79 (1H, d, J = 2.7 Hz), 5.92 (1H, dd, J = 7.5, 2.8 Hz), 6.26 (1H, t, J = 6.0 |
| | Hz), 6.63 (2H, s), 6.72 (1H, d, J = 7.7 Hz), 7.19 (1H, d, J = 6.2 Hz), 7.23 (1H, t, J = 8.0 Hz), 7.39 |
| | (1H, d, J = 8.3 Hz), 7.55 (1H, d, J = 7.6 Hz), 7.73 (1H, d, J = 6.2 Hz), 8.35 (2H, s) 2 acid |
| | protons and an NH missing |
| 12008 | 1.69 (2H, d, J = 3.1 Hz), 1.97 (2H, d, J = 4.2 Hz), 3.31 (3H, s), 3.61 (2H,s), 3.98 (2H, d, J = 6.0 |
| | Hz), 4.18 (2H, s), 5.79 (1H, d, J = 2.8 Hz), 5.90 (1H, dd, J = 7.5, 2.6 Hz), 6.01 (1H, t, J = 6.2 |
| | Hz), 6.54 (2H, s), 6.73 (1H, d, J = 7.8 Hz), 7.00 (1H, d, J = 5.5 Hz), 7.12-7.25 (4H, m), 7.35 |
| | (1H, d, J = 8.4 Hz), 7.38-7.43 (2H, m), 7.53 (1H, d, J = 7.6 Hz), 7.73 (1H, d, J = 6.1 Hz), 8.28 |
| | (1H, s) acid proton missing |
| 12009 | 1.51-1.59 (2H, m), 1.84-1.95 (2H, m), 2.00 (3H, s), 3.31 (3H, s), 3.49 (2H, s), 3.95 (2H, d, J = |
| | 6.2 Hz), 4.14 (2H, s), 5.78 (1H, d, J = 2.7 Hz), 5.89 (1H, dd, J = 7.5, 2.7 Hz), 6.25 (1H, t, J = |
| | 6.8 Hz), 6.55 (2H, s), 6.79 (1H, d, J = 7.7 Hz), 6.82 (1H, d, J = 6.2 Hz), 7.21 (1H, t, J = 8.0 Hz), |
| | 7.33 (1H, d, J = 8.3 Hz), 7.54 (1H, d, J = 7.5 Hz), 7.72 (1H, d, J = 6.1 Hz), 8.30 (1H, s). Formic |
| | acid OH not seen |
| 12010 | 1.52-1.60 (2H, m), 1.88-1.98 (2H, m), 3.31 (3H, s), 3.44 (2H, s), 3.87 (2H, d, J = 5.9 Hz), |
| | 4.15 (2H, s), 5.09 (2H, s), 5.78 (1H, d, J = 2.8 Hz), 5.90 (1H, dd, J = 7.5, 2.8 Hz), 6.26 (1H, t, J = |
| | 6.1 Hz), 6.48-6.59 (1H, m), 6.64 (1H, d, J = 6.0 Hz), 6.91 (1H, d, J = 2.3 Hz), 6.93 (2H, d, J = |
| | 2.3 Hz), 7.27-7.41 (5H, m), 7.51 (1H, d, J = 6.1 Hz), 7.54 (1H, d, J = 7.5 Hz), 7.90 (1H, d, J = |
| | 9.1 Hz), 8.32 (1H, s) |
| 12011 | 1.34-1.47 (2H, m), 1.60-1.76 (2H, m), 2.90 (2H, s), 3.31 (3H, s), 4.15 (2H, s), 5.78 (1H, d, J = |
| | 2.8 Hz), 5.91 (1H, dd, J = 7.5, 2.7 Hz), 6.15 (1H, t, J = 5.7 Hz), 6.32 (2H, s), 6.52 (1H, d, J = |
| | 2.3 Hz), 6.59 (1H, d, J = 5.9 Hz), 6.88 (1H, dd, J = 9.0, 2.4 Hz), 7.53 (1H, d, J = 3.6 Hz), 7.55 |
| | (1H, d, J = 1.9 Hz), 7.84 (1H, d, J = 9.0 Hz) |
| 12012 | 1.49-1.63 (2H, m), 1.93 (2H, d, J = 5.5 Hz), 3.04 (3H, s), 3.47 (2H, s), 3.95 (2H, d, J = 6.0 Hz), |
| | 4.23 (2H, s), 4.35 (2H, s), 5.12 (2H, s), 5.93-6.06 (1H, m), 6.56 (2H, s), 6.71 (1H, d, J = 7.8 |
| | Hz), 6.92 (1H, d, J = 6.1 Hz), 7.10 (1H, dd, J = 8.3, 2.5 Hz), 7.14 (1H, d, J = 2.4 Hz), 7.21 (1H, t, |
| | J = 8.0 Hz), 7.26-7.48 (7H, m), 7.70 (1H, d, J = 6.1 Hz), 8.19 (1H, s). Formic acid OH not |
| | seen |
| 12013 | 1.55 (2H, d, J = 4.4 Hz), 1.90 (2H, d, J = 4.6 Hz), 3.31 (3H, s), 3.38 (2H, s), 3.60 (3H, s), 3.87 |
| | (2H, d, J = 6.1 Hz), 4.15 (2H, s), 5.78 (1H, d, J = 2.8 Hz), 5.90 (1H, dd, J = 7.5, 2.7 Hz), 6.25 |
| | (1H, t, J = 6.3 Hz), 6.59 (1H, d, J = 2.4 Hz), 6.65 (1H, d, J = 6.1 Hz), 6.93 (3H, dd, J = 9.2, 2.4 |
| | Hz), 7.50 (1H, d, J = 6.2 Hz), 7.54 (1H, d, J = 7.5 Hz), 7.89 (1H, d, J = 9.0 Hz), 8.33 (1H, s) |
| 12014 | 1.65 (2H, dd, J = 4.8, 1.9 Hz), 1.86-1.93 (2H, m), 3.04-3.11 (2H, m), 3.31 (3H, s), 3.45- |
| | 3.49 (4H, m), 3.81 (2H, d, J = 6.1 Hz), 4.14 (2H, s), 5.78 (1H, d, J = 2.7 Hz), 5.86-5.93 (2H, |
| | m), 6.53 (2H, d, J = 5.3 Hz), 6.69 (1H, d, J = 7.7 Hz), 6.99-7.04 (1H, m), 7.17-7.31 (6H, m), |
| | 7.37 (1H, d, J = 8.3 Hz), 7.53 (1H, d, J = 7.6 Hz), 7.74 (1H, d, J = 6.1 Hz) |
| 12015 | 1.63-1.72 (2H, m), 1.95-2.03 (2H, m), 3.31 (3H, s), 3.66 (2H, s), 3.98 (2H, d, J = 6.3 Hz), |
| | 4.20 (2H, s), 5.79 (1H, d, J = 2.8 Hz), 5.90 (1H, dd, J = 7.5, 2.8 Hz), 5.97 (1H, s), 6.52 (2H, s), |
| | 6.72 (1H, d, J = 7.7 Hz), 6.98 (1H, s), 7.22 (1H, t, J = 8.0 Hz), 7.29 (1H, ddd, J = 8.7, 6.5, 2.5 |
| | Hz), 7.35 (1H, d, J = 8.3 Hz), 7.39 (2H, dd, J = 6.7, 1.8 Hz), 7.53 (1H, d, J = 7.6 Hz), 7.56-7.60 |
| | (1H, m), 7.72 (1H, d, J = 6.1 Hz) |
| 12016 | 1.43 (2H, dd, J = 4.5, 1.8 Hz), 1.81-1.90 (2H, m), 2.57 (2H, t, J = 7.5 Hz), 2.88 (2H, t, J = 7.5 |
| | Hz), 3.30 (3H, s), 3.37 (2H, s), 3.97 (2H, d, J = 6.6 Hz), 4.08 (2H, s), 5.75 (1H, d, J = 2.7 Hz), |
| | 5.86 (1H, dd, J = 7.5, 2.7 Hz), 6.21 (1H, t, J = 6.6 Hz), 6.51 (2H, s), 6.80 (2H, dd, J = 15.9, 6.9 |
| | Hz), 7.13-7.27 (6H, m), 7.33 (1H, d, J = 8.3 Hz), 7.53 (1H, d, J = 7.5 Hz), 7.72 (1H, d, J = 6.0 Hz) |
| 12017 | 1.49 (2H, d, J = 3.9 Hz), 1.82 (2H, d, J = 4.6 Hz), 3.30 (3H, s), 3.36 (2H, s), 3.94 (2H, d, J = 6.4 |
| | Hz), 4.15 (2H, s), 4.27 (2H, d, J = 6.0 Hz), 5.78 (1H, d, J = 2.8 Hz), 5.88 (1H, dd, J = 7.5, 2.8 |
| | Hz), 6.29 (1H, t, J = 6.7 Hz), 6.48 (2H, s), 6.75 (1H, d, J = 7.8 Hz), 6.85 (1H, d, J = 6.2 Hz), 6.99 |
| | (1H, t, J = 6.0 Hz), 7.19 (1H, t, J = 8.0 Hz), 7.22-7.27 (1H, m), 7.31 (5H, d, J = 5.6 Hz), 7.52 |
| | (1H, d, J = 7.6 Hz), 7.67 (1H, d, J = 6.1 Hz) |
| 12018 | 1.54-1.61 (2H, m), 1.90-1.96 (2H, m), 3.01 (3H, s), 3.48 (2H, s), 3.95 (2H, d, J = 6.1 Hz), |
| | 4.24 (2H, s), 4.35 (2H, s), 5.12 (2H, s), 6.01 (1H, s), 6.55 (2H, s), 6.71 (1H, d, J = 7.8 Hz), 6.92 |
| | (1H, d, J = 6.1 Hz), 6.97 (1H, dd, J = 8.4, 2.3 Hz), 7.10 (1H, d, J = 2.2 Hz), 7.20 (1H, t, J = 8.0 |
| | Hz), 7.28-7.45 (6H, m), 7.51 (1H, d, J = 8.4 Hz), 7.71 (1H, d, J = 6.0 Hz), 8.25 (1H, s) |
| 12019 | 1.61 (4H, q, J = 5.9 Hz), 1.72 (2H, p, J = 7.1 Hz), 2.52 (2H, d, J = 5.7 Hz), 2.64-2.68 (2H, m), |
| | 2.72 (2H, s), 3.31 (3H, s), 3.36 (2H, d, J = 5.2 Hz), 4.09 (2H, s), 5.58 (1H, t, J = 5.3 Hz), 5.76 |
| | (1H, d, J = 2.8 Hz), 5.89 (1H, dd, J = 7.5, 2.8 Hz), 6.52 (2H, s), 6.57 (1H, d, J = 7.7 Hz), 6.98 |
| | (1H, d, J = 5.9 Hz), 7.09-7.14 (1H, m), 7.15-7.24 (5H, m), 7.35 (1H, d, J = 8.4 Hz), 7.52 (1H, |
| | d, J = 7.6 Hz), 7.73 (1H, d, J = 6.1 Hz) |
| 12020 | 1.56 (2H, dd, J = 4.6, 1.8 Hz), 1.91 (2H, d, J = 4.7 Hz), 3.30 (3H, s), 3.44 (2H, s), 3.93 (2H, d, J = |
| | 6.3 Hz), 4.13 (2H, s), 5.09 (2H, s), 5.76 (1H, d, J = 2.7 Hz), 5.87 (1H, dd, J = 7.5, 2.7 Hz), |
| | 5.95 (1H, s), 6.51 (2H, s), 6.69 (1H, d, J = 7.7 Hz), 6.90 (1H, d, J = 6.2 Hz), 7.20 (1H, t, J = 8.0 |
| | Hz), 7.27 (1H, s), 7.34 (1H, d, J = 8.3 Hz), 7.40-7.55 (3H, m), 7.70 (1H, d, J = 6.1 Hz) |
| 12021 | 1.59 (2H, dd, J = 4.4, 1.8 Hz), 1.89-1.95 (2H, m), 3.31 (3H, s), 3.49 (2H, s), 3.96 (2H, d, J = |
| | 6.3 Hz), 4.16 (2H, s), 5.19 (2H, s), 5.78 (1H, d, J = 2.8 Hz), 5.89 (1H, dd, J = 7.5, 2.8 Hz), 5.99 |
| | (1H, t, J = 6.5 Hz), 6.51 (2H, s), 6.72 (1H, d, J = 7.8 Hz), 6.94 (1H, d, J = 6.1 Hz), 7.21 (1H, t, J = |
| | 8.0 Hz), 7.31-7.37 (2H, m), 7.43 (1H, d, J = 7.8 Hz), 7.53 (1H, d, J = 7.6 Hz), 7.71 (1H, d, J = |
| | 6.1 Hz), 7.80-7.85 (1H, m), 8.56 (1H, ddd, J = 4.8, 1.8, 0.9 Hz) |
| 12022 | 1.56 (2H, d, J = 2.9 Hz), 1.91 (2H, d, J = 4.1 Hz), 3.30 (3H, s), 3.45 (2H, s), 3.92 (2H, s), 4.13 |
| | (2H, s), 5.19 (2H, s), 5.75 (1H, d, J = 2.8 Hz), 5.87 (1H, dd, J = 7.5, 2.8 Hz), 5.96 (1H, t, J = 6.0 |
| | Hz), 6.50 (2H, s), 6.67 (1H, s), 6.92 (1H, d, J = 6.0 Hz), 7.20 (1H, t, J = 8.0 Hz), 7.33 (1H, d, J = |
| | 8.3 Hz), 7.35-7.42 (2H, m), 7.45-7.57 (3H, m), 7.70 (1H, d, J = 6.1 Hz) |
| 12023 | 1.50 (2H, d, J = 2.8 Hz), 1.85 (2H, d, J = 4.8 Hz), 3.25 (3H, s), 3.39 (2H, s), 3.89 (2H, d, J = 6.2 |
| | Hz), 4.08 (2H, s), 5.11 (2H, s), 5.71 (1H, d, J = 2.8 Hz), 5.82 (1H, dd, J = 7.5, 2.8 Hz), 5.93 (1H, |
| | t, J = 6.6 Hz), 6.46 (2H, s), 6.65 (1H, d, J = 7.4 Hz), 6.86 (1H, d, J = 6.1 Hz), 7.15 (1H, t, J = 8.0 |
| | Hz), 7.29 (1H, d, J = 8.3 Hz), 7.35 (1H, dd, J = 7.5, 5.0 Hz), 7.47 (1H, d, J = 7.6 Hz), 7.66 (1H, |
| | d, J = 6.1 Hz), 7.78 (1H, dt, J = 7.9, 2.0 Hz), 8.49 (1H, dd, J = 4.8, 1.7 Hz), 8.59 (1H, d, J = 1.6 Hz) |
| 12024 | 1.56 (2H, dd, J = 4.6, 1.8 Hz), 1.91 (2H, d, J = 4.6 Hz), 3.30 (3H, s), 3.46 (2H, s), 3.95 (2H, d, J = |
| | 6.3 Hz), 4.14 (2H, s), 5.29 (2H, s), 5.76 (1H, d, J = 2.7 Hz), 5.87 (1H, dd, J = 7.6, 2.8 Hz), |
| | 5.96 (1H, s), 6.49 (2H, s), 6.69 (1H, s), 6.86 (1H, d, J = 6.1 Hz), 7.17 (1H, t, J = 8.0 Hz), 7.32 |
| | (1H, d, J = 8.3 Hz), 7.50-7.57 (4H, m), 7.60 (1H, d, J = 6.1 Hz), 7.90-7.94 (4H, m) |
| 12025 | 1.49-1.58 (2H, m), 1.79-1.89 (2H, m), 3.30 (3H, s), 3.43 (2H, s), 3.92-4.00 (2H, m), 4.13 |
| | (2H, s), 5.10 (2H, s), 5.76 (1H, d, J = 2.6 Hz), 5.88 (1H, dd, J = 7.6, 2.7 Hz), 6.15 (1H, d, J = 5.5 |
| | Hz), 6.44 (1H, s), 6.53 (1H, s), 7.25-7.46 (5H, m), 7.52 (1H, d, J = 7.5 Hz), 7.75 (1H, d, J = 5.7 Hz) |
| 12026 | 1.51 (2H, dd, J = 4.4, 1.8 Hz), 1.88 (2H, d, J = 4.8 Hz), 3.30 (3H, s), 3.35 (2H, s), 3.80 (3H, s), |
| | 3.93 (2H, d, J = 6.3 Hz), 4.11 (2H, s), 4.96 (2H, s), 5.72-5.77 (1H, m), 5.87 (1H, dd, J = 7.5, |
| | 2.7 Hz), 5.99 (1H, s), 6.51 (2H, s), 6.70 (1H, d, J = 7.7 Hz), 6.92 (1H, d, J = 6.1 Hz), 7.20 (1H, t, |
| | J = 8.1 Hz), 7.34 (1H, d, J = 8.3 Hz), 7.46 (1H, s), 7.51 (1H, d, J = 7.5 Hz), 7.72 (2H, d, J = 6.3 Hz) |
| 12027 | 1.43 (9H, s), 1.48-1.53 (2H, m), 1.83-1.91 (2H, m), 2.54 (2H, s), 3.30 (3H, s), 3.89 (2H, d, J = |
| | 5.9 Hz), 4.11 (2H, s), 5.75 (1H, d, J = 2.8 Hz), 5.88 (1H, dd, J = 7.5, 2.8 Hz), 6.59 (2H, s), |
| | 6.71 (1H, d, J = 7.7 Hz), 6.97 (1H, d, J = 6.2 Hz), 7.22 (1H, t, J = 8.0 Hz), 7.34 (1H, d, J = 8.3 |
| | Hz), 7.52 (1H, d, J = 7.6 Hz), 7.71 (1H, d, J = 6.1 Hz), 8.21 (1H, s) |
| 12028 | 1.51 (2H, dd, J = 4.5, 1.8 Hz), 1.86-1.94 (2H, m), 3.31 (3H, s), 3.58 (2H, s), 3.66 (2H, s), 3.98 |
| | (2H, d, J = 6.5 Hz), 4.13 (2H, s), 5.77 (1H, d, J = 2.7 Hz), 5.87 (1H, dd, J = 7.5, 2.7 Hz), 6.15 |
| | (1H, t, J = 6.7 Hz), 6.54 (2H, s), 6.79 (2H, dd, J = 7.1, 4.0 Hz), 7.17-7.27 (2H, m), 7.24-7.35 |
| | (5H, m), 7.53 (1H, d, J = 7.5 Hz), 7.69 (1H, d, J = 6.1 Hz) |
| 12031 | 0.96 (2H, q, J = 11.9 Hz), 1.12 (1H, q, J = 10.9 Hz), 1.19-1.27 (2H, m), 1.43-1.52 (2H, m), |
| | 1.64 (3H, t, J = 14.7 Hz), 1.73 (2H, d, J = 12.7 Hz), 1.76-1.85 (1H, m), 1.86-1.92 (2H, m), |
| | 2.14 (2H, d, J = 6.9 Hz), 3.31 (3H, s), 3.48 (2H, s), 3.97 (2H, d, J = 6.6 Hz), 4.13 (2H, s), 5.77 |
| | (1H, d, J = 2.7 Hz), 5.89 (1H, dd, J = 7.5, 2.7 Hz), 6.21 (1H, t, J = 6.7 Hz), 6.51 (2H, s), 6.79 |
| | (1H, d, J = 6.2 Hz), 6.81 (1H, d, J = 7.9 Hz), 7.20 (1H, t, J = 8.0 Hz), 7.32 (1H, d, J = 8.3 Hz), |
| | 7.53 (1H, d, J = 7.6 Hz), 7.70 (1H, d, J = 6.1 Hz) |
| 12034 | 1.50 (2H, d, J = 4.4 Hz), 1.90 (2H, d, J = 4.7 Hz), 3.30 (3H, s), 3.60 (2H, s), 3.74 (2H, s), 3.99 |
| | (2H, d, J = 6.5 Hz), 4.13 (2H, s), 5.77 (1H, d, J = 2.8 Hz), 5.87 (1H, dd, J = 7.5, 2.8 Hz), 6.16 |
| | (1H, t, J = 6.7 Hz), 6.53 (2H, s), 6.78 (2H, dd, J = 13.3, 7.0 Hz), 6.90 (1H, dd, J = 2.2, 1.0 Hz), |
| | 7.19 (1H, t, J = 7.9 Hz), 7.25 (1H, dd, J = 8.4, 1.8 Hz), 7.32 (1H, d, J = 8.3 Hz), 7.50-7.55 (2H, |
| | m), 7.57 (1H, d, J = 1.8 Hz), 7.67 (1H, d, J = 6.1 Hz), 7.97 (1H, d, J = 2.2 Hz) |
| 12035 | 1.64 (2H, dd, J = 4.6, 1.8 Hz), 1.87-1.98 (2H, m), 3.28 (3H, s), 3.39 (2H, s), 4.07 (4H, s), 5.71 |
| | (1H, d, J = 2.8 Hz), 5.83 (1H, dd, J = 7.5, 2.7 Hz), 6.22 (1H, t, J = 6.4 Hz), 6.51 (2H, s), 6.78 |
| | (1H, d, J = 7.7 Hz), 6.96-7.03 (1H, m), 7.23 (1H, t, J = 8.0 Hz), 7.35 (1H, d, J = 8.3 Hz), 7.43- |
| | 7.52 (4H, m), 7.62 (2H, dd, J = 7.6, 1.9 Hz), 7.73 (1H, d, J = 6.1 Hz) |
| 12036 | 1.58 (2H, dd, J = 4.5, 1.8 Hz), 1.80-1.90 (2H, m), 3.31 (3H, s), 3.54 (2H, s), 4.01 (2H, d, J = |
| | 6.3 Hz), 4.17 (2H, s), 5.79 (1H, d, J = 2.7 Hz), 5.89 (1H, dd, J = 7.5, 2.8 Hz), 6.20 (1H, t, J = 6.5 |
| | Hz), 6.50 (2H, s), 6.75 (1H, dd, J = 7.8, 0.9 Hz), 6.91-6.97 (1H, m), 6.98 (1H, d, J = 6.2 Hz), |
| | 7.23 (3H, td, J = 8.9, 7.6 Hz), 7.33 (1H, d, J = 8.3 Hz), 7.52 (3H, ddd, J = 7.9, 2.3, 1.3 Hz), 7.72 |
| | (1H, d, J = 6.1 Hz), 8.51 (1H, s) |
| 12038 | 1.48 (2H, d, J = 3.0 Hz), 1.81 (2H, d, J = 3.9 Hz), 2.26 (3H, s), 3.30 (3H, s), 3.94 (2H, d, J = 6.3 |
| | Hz), 4.12 (2H, s), 4.27 (2H, d, J = 5.9 Hz), 5.69 (1H, d, J = 1.9 Hz), 5.84 (1H, d, J = 2.3 Hz), |
| | 6.31 (1H, t, J = 6.5 Hz), 6.52 (2H, s), 6.74 (1H, d, J = 7.8 Hz), 6.86 (1H, d, J = 6.2 Hz), 7.02 (1H, |
| | t, J = 6.5 Hz), 7.17-7.25 (2H, m), 7.29-7.34 (5H, m), 7.67 (1H, d, J = 6.0 Hz), 2 protons |
| | hidden under the water peak |
| 12039 | 1.49 (2H, d, J = 3.2 Hz), 1.89 (2H, d, J = 4.0 Hz), 2.26 (3H, s), 3.30 (3H, s), 3.57 (2H, s), 3.66 |
| | (2H, s), 3.98 (2H, d, J = 6.4 Hz), 4.11 (2H, s), 6.58 (1H, d, J = 2.7 Hz), 5.83 (1H, d, J = 2.4 Hz), |
| | 6.16 (1H, t, J = 6.6 Hz), 6.54 (2H, s), 6.79 (2H, d, J = 6.3 Hz), 7 .. 18-7.34 (7H, m), 7.70 (1H, d, |
| | J = 6.1 Hz) |
| 12040 | 1.50 (2H, dd, J = 4.6, 1.8 Hz), 1.85-1.93 (2H, m), 3.33 (3H, s), 3.57 (2H, s), 3.87 (2H, s), 3.98 |
| | (2H, d, J = 6.6 Hz), 4.11 (2H, s), 5.75 (1H, d, J = 2.8 Hz), 5.85 (1H, dd, J = 7.5, 2.8 Hz), 6.15 |
| | (1H, t, J = 6.7 Hz), 6.54 (3H, d, J = 11.7 Hz), 6.73-6.83 (2H, m), 6.90 (1H, d, J = 7.1 Hz), 7.01 |
| | (1H, dd, J = 8.1, 7.1 Hz), 7.19 (1H, t, J = 8.0 Hz), 7.25-7.35 (3H, m), 7.52 (1H, d, J = 7.6 Hz), |
| | 7.67 (1H, d, J = 6.1 Hz), 11.11 (1H, s) |
| 12041 | 1.57-1.64 (2H, m), 1.72-1.79 (2H, m), 2.82 (3H, s), 3.30 (3H, s), 3.30 (2H, s), 3.85 (2H, d, J = |
| | 6.1 Hz), 4.12 (2H, s), 4.50 (2H, s), 5.74 (1H, d, J = 2.8 Hz), 5.87 (1H, dd, J = 7.5, 2.8 Hz), |
| | 6.16 (1H, t, J = 6.2 Hz), 6.47 (2H, s), 6.74 (1H, d, J = 7.8 Hz), 7.06-7.11 (1H, m), 7.18 (1H, t, J = |
| | 8.0 Hz), 7.23-7.39 (6H, m), 7.51 (1H, d, J = 7.5 Hz), 7.71 (1H, d, J = 6.1 Hz) |
| 12042 | 1.41-1.48 (2H, m), 1.82-1.89 (2H, m), 2.57 (2H, t, J = 7.5 Hz), 2.86 (2H, t, J = 7.5 Hz), 3.30 |
| | (3H, s), 3.38 (2H, s), 3.70 (3H, s), 3.97 (2H, d, J = 6.5 Hz), 4.08 (2H, s), 5.75 (1H, d, J = 2.8 |
| | Hz), 5.86 (1H, dd, J = 7.5, 2.8 Hz), 6.22 (1H, t, J = 6.7 Hz), 6.51 (2H, s), 6.72-6.88 (5H, m), |
| | 7.15 (1H, t, J = 8.0 Hz), 7.21 (1H, t, J = 8.0 Hz), 7.33 (1H, d, J = 8.3 Hz), 7.53 (1H, d, J = 7.5 |
| | Hz), 7.72 (1H, d, J = 6.1 Hz) |
| 12044 | 1.42-1.51 (2H, m), 1.84-1.91 (2H, m), 2.61-2.69 (2H, m), 2.94 (2H, t, J = 7.4 Hz), 3.31 |
| | (3H, s), 3.43 (2H, s), 3.97 (2H, d, J = 6.5 Hz), 4.11 (2H, s), 5.76 (1H, d, J = 2.7 Hz), 5.87 (1H, |
| | dd, J = 7.5, 2.8 Hz), 6.17 (1H, t, J = 6.6 Hz), 6.51 (2H, s), 6.77 (2H, t, J = 6.5 Hz), 7.20 (1H, t, J = |
| | 8.0 Hz), 7.33 (1H, d, J = 8.3 Hz), 7.46 (1H, t, J = 7.7 Hz), 7.53 (1H, d, J = 7.6 Hz), 7.62-7.69 |
| | (2H, m), 7.71 (1H, d, J = 6.1 Hz), 7.78 (1H, s) |
| 12045 | 1.41 (9H, s), 1.53-1.61 (2H, m), 1.86-1.95 (2H, m), 3.31 (5H, s), 3.85 (2H, d, J = 5.7 Hz), |
| | 4.15 (2H, s), 5.78 (1H, d, J = 2.7 Hz), 5.90 (1H, dd, J = 7.5, 2.8 Hz), 6.45 (1H, t, J = 5.9 Hz), |
| | 6.66 (1H, d, J = 2.2 Hz), 7.16 (1H, dd, J = 8.9, 2.2 Hz), 7.42 (1H, d, J = 5.8 Hz), 7.54 (1H, d, J = |
| | 7.6 Hz), 7.74 (1H, d, J = 8.9 Hz), 8.17 (1H, d, J = 5.8 Hz), 8.85 (1H, s) |
| 12046 | 1.43 (9H, s), 1.51-1.54 (2H, m), 1.88 (2H, d, J = 4.6 Hz), 2.25 (3H, s), 3.30 (3H, s), 3.34 (2H, |
| | s), 3.96 (2H, d, J = 6.1 Hz), 4.10 (2H, s), 5.66 (1H, d, J = 2.8 Hz), 5.80-5.85 (1H, m), 6.42 (1H, |
| | s), 6.78 (1H, d, J = 7.8 Hz), 7.27 (1H, d, J = 8.1 Hz), 7.44 (1H, t, J = 7.9 Hz), 7.93 (1H, d, J = 6.1 |
| | Hz), 8.41 (1H, d, J = 5.9 Hz), 9.13 (1H, d, J = 0.8 Hz) |
| 12047 | 1.29-1.36 (2H, m), 1.78-1.83 (2H, m), 2.70 (2H, t, J = 7.4 Hz), 3.24 (2H, s), 3.30 (3H, s), |
| | 3.37 (2H, t, J = 7.3 Hz), 3.96 (2H, d, J = 6.6 Hz), 4.01 (2H, s), 5.71 (1H, d, J = 2.8 Hz), 5.81 (1H, |
| | dd, J = 7.5, 2.7 Hz), 6.21 (1H, t, J = 6.8 Hz), 6.52 (2H, s), 6.76 (1H, d, J = 7.8 Hz), 6.82 (1H, d, J = |
| | 6.1 Hz), 7.21 (1H, t, J = 8.0 Hz), 7.33 (1H, d, J = 8.3 Hz), 7.50-7.58 (2H, m), 7.68-7.74 (2H, |
| | m), 7.96-8.02 (2H, m), 8.53 (1H, d, J = 6.0 Hz), 9.31 (1H, d, J = 0.9 Hz) |
| 12048 | 1.39-1.46 (2H, m), 1.83-1.89 (2H, m), 2.62 (2H, t, J = 7.3 Hz), 2.95 (2H, t, J = 7.5 Hz), 3.30 |
| | (3H, s), 3.40 (2H, s), 3.97 (2H, d, J = 6.5 Hz), 4.08 (2H, s), 5.75 (1H, d, J = 2.8 Hz), 5.85 (1H, |
| | dd, J = 7.5, 2.7 Hz), 6.20 (1H, t, J = 6.7 Hz), 6.51 (2H, s), 6.78 (1H, d, J = 7.8 Hz), 6.81 (1H, d, J = |
| | 6.2 Hz), 6.95 (1H, t, J = 55.9 Hz), 7.20 (1H, t, J = 8.0 Hz), 7.33 (1H, d, J = 8.3 Hz), 7.37-7.42 |
| | (2H, m), 7.46 (1H, d, J = 5.9 Hz), 7.48 (1H, s), 7.53 (1H, d, J = 7.6 Hz), 7.71 (1H, d, J = 6.0 Hz) |
| 12049 | 1.45 (2H, d, J = 4.3 Hz), 1.87 (2H, d, J = 4.3 Hz), 2.60 (2H, t, J = 7.5 Hz), 2.89 (2H, t, J = 7.5 |
| | Hz), 3.30 (3H, s), 3.40 (2H, s), 3.97 (2H, d, J = 6.6 Hz), 4.09 (2H, s), 5.76 (1H, d, J = 2.7 Hz), |
| | 5.86 (1H, dd, J = 7.5, 2.8 Hz), 6.19 (1H, t, J = 6.7 Hz), 6.51 (2H, s), 6.79 (2H, t, J = 6.9 Hz), |
| | 7.20 (1H, t, J = 8.0 Hz), 7.23-7.30 (3H, m), 7.33 (1H, d, J = 8.3 Hz), 7.36 (1H, s), 7.53 (1H, d, |
| | J = 7.6 Hz), 7.71 (1H, d, J = 6.0 Hz) |
| 12050 | 1.44 (2H, d, J = 4.4 Hz), 1.86 (2H, d, J = 4.6 Hz), 2.61-2.69 (2H, m), 2.99 (2H, t, J = 7.5 Hz), |
| | 3.30 (3H, s), 3.42 (2H, s), 3.96 (2H, d, J = 6.6 Hz), 4.09 (2H, s), 5.75 (1H, d, J = 2.8 Hz), 5.85 |
| | (1H, dd, J = 7.5, 2.7 Hz), 6.18 (1H, t, J = 6.6 Hz), 6.51 (2H, s), 6.78 (2H, t, J = 6.9 Hz), 7.20 |
| | (1H, t, J = 8.0 Hz), 7.33 (1H, d, J = 8.3 Hz), 7.49 (1H, t, J = 7.7 Hz), 7.53 (1H, d, J = 7.6 Hz), |
| | 7.55 (1H, d, J = 8.0 Hz), 7.61 (1H, d, J = 7.6 Hz), 7.65 (1H, s), 7.70 (1H, d, J = 6.0 Hz) |
| 12051 | 1.46 (2H, s), 1.84 (2H, s), 2.55 (2H, t, J = 7.8 Hz), 2.86 (2H, t, J = 7.7 Hz), 3.30 (3H, s), 3.40 |
| | (2H, s), 3.98 (2H, d, J = 6.5 Hz), 4.07 (2H, s), 5.74 (1H, d, J = 2.8 Hz), 5.90 (1H, dd, J = 7.5, 2.7 |
| | Hz), 6.23 (1H, s), 6.50 (2H, s), 6.78 (1H, d, J = 7.8 Hz), 6.85 (1H, d, J = 6.3 Hz), 7.08 (1H, t, J = |
| | 7.3 Hz), 7.12 (1H, d, J = 7.3 Hz), 7.21 (1H, t, J = 8.0 Hz), 7.32 (1H, d, J = 8.3 Hz), 7.52 (1H, d, J = |
| | 7.6 Hz), 7.60-7.66 (1H, m), 7.70-7.74 (2H, m). One exchangeable proton is not |
| | observed |
| 12052 | 1.23-1.27 (2H, m), 1.71-1.76 (2H, m), 2.62 (2H, t, J = 7.4 Hz), 3.15 (2H, s), 3.23 (3H, s), |
| | 3.33 (2H, t, J = 7.3 Hz), 3.89 (2H, d, J = 6.5 Hz), 3.94 (2H, s), 5.64 (1H, d, J = 2.8 Hz), 5.75 (1H, |
| | dd, J = 7.5, 2.8 Hz), 6.14 (1H, t, J = 6.7 Hz), 6.44 (2H, d, J = 5.3 Hz), 6.69 (1H, d, J = 7.8 Hz), |
| | 6.75 (1H, d, J = 6.2 Hz), 7.14 (1H, t, J = 8.0 Hz), 7.26 (1H, d, J = 8.3 Hz), 7.43-7.51 (3H, m), |
| | 7.55 (1H, dd, J = 8.5, 7.1 Hz), 7.65 (1H, d, J = 6.1 Hz), 7.82 (1H, d, J = 8.4 Hz), 8.52 (1H, d, J = |
| | 8.5 Hz), 8.83 (1H, dd, J = 4.1, 1.6 Hz) |
| 12053 | 1.45 (2H, d, J = 4.8 Hz), 1.84-1.89 (2H, m), 2.61 (2H, q, J = 7.7 Hz), 2.88 (2H, t, J = 7.4 Hz), |
| | 3.31 (3H, s), 3.40 (2H, s), 3.97 (2H, d, J = 6.6 Hz), 4.09 (2H, s), 5.76 (1H, d, J = 2.8 Hz), 5.86 |
| | (1H, dd, J = 7.5, 2.8 Hz), 6.19 (1H, t, J = 6.6 Hz), 6.50 (2H, d, J = 5.4 Hz), 6.79 (2H, t, J = 7.1 |
| | Hz), 7.21 (2H, td, J = 7.9, 4.1 Hz), 7.26-7.41 (3H, m), 7.48-7.56 (2H, m), 7.72 (1H, d, J = 6.1 Hz) |
| 12054 | 1.46-1.51 (2H, m), 1.86-1.91 (2H, m), 2.62 (2H, t, J = 7.3 Hz), 3.32 (3H, s), 3.98 (2H, d, J = |
| | 6.6 Hz), 4.12 (2H, s), 5.77 (1H, d, J = 2.8 Hz), 5.89 (1H, dd, J = 7.5, 2.8 Hz), 6.19 (1H, d, J = |
| | 7.0 Hz), 6.24 (1H, d, J = 9.3 Hz), 6.51-6.52 (2H, m), 6.79 (2H, d, J = 7.5 Hz), 7.18-7.25 (2H, |
| | m), 7.34 (1H, d, J = 8.3 Hz), 7.42 (1H, dd, J = 9.3, 2.7 Hz), 7.54 (1H, d, J = 7.6 Hz), 7.73 (1H, d, |
| | J = 6.1 Hz), 11.38 (1H, s). 2H are under DMSO peak |
| 12055 | 1.42-1.47 (2H, m), 1.81-1.87 (2H, m), 2.74 (2H, t, J = 7.4 Hz), 3.12 (2H, t, J = 7.4 Hz), 3.30 |
| | (3H, s), 3.44 (2H, s), 3.98 (2H, d, J = 6.6 Hz), 4.08 (2H, s), 5.74 (1H, d, J = 2.8 Hz), 5.81 (1H, |
| | dd, J = 7.5, 2.8 Hz), 6.18 (1H, t, J = 6.7 Hz), 6.50 (2H, d, J = 5.4 Hz), 6.77 (2H, t, J = 6.9 Hz), |
| | 7.20 (1H, t, J = 8.0 Hz), 7.32 (1H, d, J = 8.3 Hz), 7.43-7.50 (1H, m), 7.52 (1H, d, J = 7.5 Hz), |
| | 7.55-7.63 (2H, m), 7.86-7.92 (2H, m), 8.32 (1H, dd, J = 8.3, 1.8 Hz), 8.87 (1H, dd, J = 4.2, |
| | 1.8 Hz) |
| 12056 | 1.44-1.49 (2H, m), 1.84-1.93 (2H, m), 2.62 (2H, q, J = 7.2 Hz), 2.91 (2H, t, J = 7.5 Hz), 3.31 |
| | (3H, s), 3.43 (2H, s), 3.98 (2H, d, J = 6.5 Hz), 4.10 (2H, s), 5.76 (1H, d, J = 2.8 Hz), 5.87 (1H, |
| | dd, J = 7.5, 2.8 Hz), 6.20 (1H, t, J = 6.6 Hz), 6.52 (2H, s), 6.80 (2H, dd, J = 14.7, 7.0 Hz), 7.01 |
| | (1H, dd, J = 8.0, 2.5 Hz), 7.14-7.25 (4H, m), 7.27-7.38 (2H, m), 7.54 (1H, d, J = 7.5 Hz), |
| | 7.72 (1H, d, J = 6.1 Hz) |
| 12057 | 1.67 (2H, dd, J = 4.7, 1.8 Hz), 1.97 (2H, d, J = 4.8 Hz), 3.27 (3H, s), 3.66 (2H, s), 4.11-4.21 |
| | (4H, m), 5.76 (1H, d, J = 2.8 Hz), 5.86 (1H, dd, J = 7.5, 2.8 Hz), 6.47 (3H, s), 6.77 (1H, d, J = |
| | 7.7 Hz), 6.97-7.00 (1H, m), 7.02 (1H, td, J = 7.7, 1.3 Hz), 7.13-7.17 (1H, m), 7.20 (1H, d, J = |
| | 7.9 Hz), 7.30 (1H, d, J = 8.4 Hz), 7.32-7.35 (1H, m), 7.39 (1H, ddd, J = 7.9, 1.2, 0.6 Hz), 7.49 |
| | (1H, d, J = 7.6 Hz), 7.66 (1H, d, J = 6.0 Hz) |
| 12058 | 1.51 (2H, dd, J = 4.4, 1.8 Hz), 1.79-1.86 (2H, m), 3.31 (3H, s), 3.39 (2H, s), 3.94 (2H, d, J = |
| | 6.4 Hz), 4.16 (2H, s), 4.32 (2H, d, J = 5.6 Hz), 5.79 (1H, d, J = 2.7 Hz), 5.89 (1H, dd, J = 7.5, |
| | 2.7 Hz), 6.26 (1H, t, J = 6.6 Hz), 6.48 (2H, d, J = 5.5 Hz), 6.75 (1H, d, J = 7.8 Hz), 6.84 (1H, d, J = |
| | 6.1 Hz), 6.98 (1H, t, J = 5.9 Hz), 7.13-7.23 (3H, m), 7.31 (2H, pd, J = 6.3, 3.5 Hz), 7.40 (1H, |
| | td, J = 7.8, 1.9 Hz), 7.53 (1H, d, J = 7.6 Hz), 7.67 (1H, d, J = 6.0 Hz) |
| 12059 | 1.59-1.67 (2H, m), 1.85-1.93 (2H, m), 3.31 (3H, s), 3.54 (2H, s), 3.71 (3H, s), 3.98 (2H, d, J = |
| | 6.1 Hz), 4.18 (2H, s), 5.79 (1H, d, J = 2.7 Hz), 5.91 (1H, dd, J = 7.5, 2.8 Hz), 6.14 (1H, t, J = |
| | 6.4 Hz), 6.51 (2H, s), 6.76 (1H, d, J = 7.8 Hz), 6.84-6.92 (1H, m), 6.95-7.02 (3H, m), 7.22 |
| | (1H, t, J = 8.0 Hz), 7.35 (1H, d, J = 8.3 Hz), 7.54 (1H, d, J = 7.6 Hz), 7.57 (1H, s), 7.72 (1H, d, J = |
| | 6.1 Hz), 7.81-7.87 (1H, m) |
| 12060 | 1.68 (2H, d, J = 3.0 Hz), 2.02 (2H, d, J = 4.3 Hz), 3.31 (3H, s), 3.61 (2H, s), 4.22 (2H, s), 4.26 |
| | (2H, d, J = 6.4 Hz), 5.81 (1H, d, J = 2.8 Hz), 5.91 (1H, dd, J = 7.5, 2.8 Hz), 6.31 (1H, t, J = 6.4 |
| | Hz), 6.50 (2H, s), 6.81 (1H, d, J = 7.7 Hz), 6.96 (1H, d, J = 6.2 Hz), 7.23 (1H, t, J = 8.0 Hz), 7.30- |
| | 7.38 (2H, m), 7.46 (2H, t, J = 7.7 Hz), 7.54 (1H, d, J = 7.6 Hz), 7.64-7.73 (3H, m), 8.75 (2H, s) |
| 12061 | 1.57 (2H, d, J = 3.0 Hz), 1.85 (2H, d, J = 4.2 Hz), 3.31 (3H, s), 3.50 (2H, s), 3.71 (3H, s), 3.99 |
| | (2H, d, J = 6.1 Hz), 4.17 (2H, s), 5.78 (1H, d, J = 2.8 Hz), 5.89 (1H, dd, J = 7.5, 2.8 Hz), 6.23 |
| | (1H, t, J = 6.5 Hz), 6.49 (2H, s), 6.75 (1H, d, J = 7.6 Hz), 6.80-6.88 (2H, m), 6.97 (1H, d, J = |
| | 6.0 Hz), 7.21 (1H, t, J = 8.0 Hz), 7.32 (1H, d, J = 8.3 Hz), 7.37-7.45 (2H, m), 7.53 (1H, d, J = |
| | 7.6 Hz), 7.71 (1H, d, J = 6.1 Hz), 8.35 (1H, s) |
| 12062 | 1.48 (2H, d, J = 2.9 Hz), 1.81 (2H, d, J = 3.6 Hz), 3.30 (3H, s), 3.33 (2H, s), 3.74 (3H, s), 3.93 |
| | (2H, d, J = 6.3 Hz), 4.14 (2H, s), 4.19 (2H, d, J = 5.7 Hz), 5.77 (1H, d, J = 2.7 Hz), 5.88 (1H, dd, |
| | J = 7.5, 2.8 Hz), 6.30 (1H, t, J = 6.6 Hz), 6.49 (2H, s), 6.75 (1H, d, J = 7.7 Hz), 6.84 (1H, d, J = |
| | 6.2 Hz), 6.85-6.89 (2H, m), 6.92 (1H, t, J = 5.9 Hz), 7.19 (1H, t, J = 8.1 Hz), 7.23 (2H, d, J = |
| | 8.7 Hz), 7.30 (1H, d, J = 8.3 Hz), 7.52 (1H, d, J = 7.6 Hz), 7.67 (1H, d, J = 6.1 Hz) |
| 12063 | 1.41 (9H, s), 1.53-1.59 (2H, m), 1.87-1.95 (2H, m), 3.30 (3H, s), 3.34 (2H, s), 3.83 (2H, d, J = |
| | 5.8 Hz), 4.14 (2H, s), 5.77 (1H, d, J = 2.8 Hz), 5.90 (1H, dd, J = 7.6, 2.8 Hz), 6.16 (1H, t, J = |
| | 6.0 Hz), 6.85 (1H, d, J = 2.3 Hz), 7.30 (1H, dd, J = 8.9, 2.3 Hz), 7.50-7.56 (2H, m), 7.65 (1H, |
| | d, J = 8.9 Hz), 8.12 (1H, d, J = 5.5 Hz), 8.93-8.97 (1H, m) |
| 12064 | 1.82 (2H, dd, J = 4.5, 1.7 Hz), 1.92 (2H, d, J = 4.4 Hz), 3.47 (3H, s), 3.89 (3H, |
| | d, J = 5.2 Hz), 4.19 (3H, s), 5.79 (1H, d, J = 2.7 Hz), 5.88 (1H, dd, J = 7.5, 2.8 Hz), 6.56 (1H, s), |
| | 6.97 (0.4H, s), 7.05 (1H, d, J = 8.1 Hz), 7.10 (0.4H, s), 7.23 (0.4H, s), 7.47 (2H, d, J = 7.3 Hz), |
| | 7.50-7.57 (2H, m), 7.62 (2H, dd, J = 7.5, 3.6 Hz), 7.70 (1H, d, J = 8.3 Hz), 8.10 (1H, dd, J = |
| | 4.9, 1.2 Hz), 8.29 (1H, d, J = 2.8 Hz), 8.80 (2H, s), 13.10 (0.8H, s) |
| 12065 | 1.77 (2H, dd, J = 4.4, 1.8 Hz), 1.95-2.00 (2H, m), 3.31 (3H, s), 3.43 (2H, s), 3.97 (2H, d, J = |
| | 5.5 Hz), 4.20 (2H, s), 5.79 (1H, d, J = 2.7 Hz), 5.89 (1H, dd, J = 7.5, 2.8 Hz), 5.95 (1H, t, J = 5.6 |
| | Hz), 6.53 (2H, s), 6.70 (3H, dd, J = 6.7, 1.6 Hz), 7.08-7.14 (1H, m), 7.24 (1H, t, J = 8.0 Hz), |
| | 7.39 (1H, d, J = 8.3 Hz), 7.53 (1H, d, J = 7.6 Hz), 7.72 (1H, d, J = 6.1 Hz), 8.03-8.09 (2H, m) |
| 12067 | 1.48-1.54 (2H, m), 1.80-1.85 (2H, m), 3.31 (3H, s), 3.40 (2H, s), 3.81 (3H, s), 3.93 (2H, d, J = |
| | 6.3 Hz), 4.16 (2H, s), 4.25 (2H, d, J = 5.8 Hz), 5.78 (1H, d, J = 2.7 Hz), 5.89 (1H, dd, J = 7.5, |
| | 2.7 Hz), 6.27 (1H, t, J = 6.5 Hz), 6.48 (2H, s), 6.74 (1H, d, J = 7.7 Hz), 6.78 (1H, t, J = 6.0 Hz), |
| | 6.84 (1H, d, J = 6.1 Hz), 6.92 (1H, td, J = 7.4, 1.1 Hz), 6.95-6.99 (1H, m), 7.16-7.26 (3H, m), |
| | 7.30 (1H, d, J = 8.3 Hz), 7.53 (1H, d, J = 7.6 Hz), 7.66 (1H, d, J = 6.1 Hz) |
| 12068 | 1.55-1.61 (2H, m), 1.84-1.90 (2H, m), 3.31 (3H, s), 3.55 (2H, s), 3.98 (2H, d, J = 6.2 Hz), |
| | 4.18 (2H, s), 5.79 (1H, d, J = 2.8 Hz), 5.90 (1H, dd, J = 7.6, 2.8 Hz), 6.18 (1H, t, J = 6.4 Hz), |
| | 6.51 (2H, s), 6.75 (1H, d, J = 7.8 Hz), 6.96 (1H, d, J = 6.2 Hz), 7.10-7.16 (2H, m), 7.17-7.24 |
| | (2H, m), 7.33 (1H, d, J = 8.3 Hz), 7.53 (1H, d, J = 7.6 Hz), 7.55-7.61 (1H, m), 7.71 (1H, d, J = |
| | 6.1 Hz), 8.25 (1H, s) |
| 12069 | 1.49 (2H, dd, J = 4.5, 1.8 Hz), 1.80-1.85 (2H, m), 3.30 (3H, s), 3.38 (2H, s), 3.93 (2H, d, J = |
| | 6.4 Hz), 4.15 (2H, s), 4.35 (2H, d, J = 5.9 Hz), 5.78 (1H, d, J = 2.8 Hz), 5.88 (1H, dd, J = 7.5, |
| | 2.7 Hz), 6.25 (1H, t, J = 6.5 Hz), 6.48 (2H, s), 6.74 (1H, d, J = 7.7 Hz), 6.81 (1H, d, J = 6.1 Hz), |
| | 7.11 (1H, t, J = 6.0 Hz), 7.18 (1H, t, J = 8.0 Hz), 7.30 (1H, d, J = 8.3 Hz), 7.53 (1H, d, J = 7.6 |
| | Hz), 7.54-7.66 (5H, m) |
| 12070 | 1.47-1.51 (2H, m), 1.79-1.85 (2H, m), 3.30 (3H, s), 3.36 (2H, s), 3.94 (2H, d, J = 6.3 Hz), |
| | 4.14 (2H, s), 4.35 (2H, d, J = 5.9 Hz), 5.77 (1H, d, J = 2.7 Hz), 5.88 (1H, dd, J = 7.5, 2.8 Hz), |
| | 6.31 (1H, t, J = 6.6 Hz), 6.50 (2H, s), 6.75 (1H, d, J = 7.7 Hz), 6.83 (1H, d, J = 6.1 Hz), 6.93 (1H, |
| | dd, J = 2.2, 1.0 Hz), 7.02 (1H, t, J = 6.0 Hz), 7.19 (1H, t, J = 8.0 Hz), 7.26-7.33 (2H, m), 7.52 |
| | (2H, d, J = 7.8 Hz), 7.57 (1H, d, J = 1.7 Hz), 7.60 (1H, s), 7.97 (1H, d, J = 2.2 Hz) |
| 12071 | 1.62 (2H, dd, J = 4.8, 1.8 Hz), 1.90 (2H, d, J = 4.7 Hz), 3.28 (4H, d, J = 2.4 Hz), 3.44 (2H, s), |
| | 3.81 (3H, s), 4.09 (3H, d, J = 5.8 Hz), 5.71 (1H, d, J = 2.8 Hz), 5.84 (1H, dd, J = 7.6, 2.8 Hz), |
| | 6.22 (1H, t, J = 6.5 Hz), 6.50 (2H, d, J = 4.3 Hz), 6.76 (1H, d, J = 7.8 Hz), 6.96-7.03 (3H, m), |
| | 7.23 (1H, t, J = 8.0 Hz), 7.34 (1H, d, J = 8.3 Hz), 7.49 (1H, d, J = 7.6 Hz), 7.60-7.66 (2H, m), |
| | 7.73 (1H, d, J = 6.1 Hz) |
| 12072 | 1.32 (2H, dd, J = 4.4, 1.8 Hz), 1.57-1.70 (2H, m), 3.92 (2H, d, J = 5.9 Hz), 4.08 (2H, s), 4.32 |
| | (2H, d, J = 5.8 Hz), 5.73 (1H, d, J = 2.7 Hz), 5.87 (1H, dd, J = 7.5, 2.7 Hz), 5.93 (1H, s), 6.75 |
| | (2H, s), 6.87 (1H, d, J = 6.2 Hz), 6.96 (1H, t, J = 5.9 Hz), 7.20 (1H, dd, J = 12.7, 9.1 Hz), 7.24- |
| | 7.29 (1H, m), 7.35 (4H, d, J = 4.4 Hz), 7.52 (1H, d, J = 7.6 Hz), 7.57-7.62 (2H, m), 8.22 (2H, s) |
| 13001 | 1.61 (2H, d, J = 3.1 Hz), 1.92 (2H, d, J = 3.1 Hz), 3.31 (3H, s), 3.45 (2H,s), 3.52 (2H, d, J = 5.8 |
| | Hz), 4.47 (2H, s), 5.01 (2H, s), 5.67 (1H, s), 5.87 (1H, dd, J = 7.5, 2.5 Hz), 6.00 (1H, t, J = 6.0 |
| | Hz), 6.49 (2H, s), 6.68 (1H, d, J = 7.7 Hz), 7.16 (1H, d, J = 6.2 Hz), 7.20 (1H, t, J = 8.0 Hz), 7.27- |
| | 7.35 (6H, m), 7.51 (1H, d, J = 7.6 Hz), 7.71 (1H, d, J = 6.1 Hz), 8.26 (1H, s) acid proton missing |
| 13002 | 1.59-1.72 (1H, m), 1.97-2.19 (3H, m), 3.83-4.21 (4H, m), 4.59-4.91 (2H, m), 4.93-5.14 |
| | (2H, m), 5.92-6.11 (1H, m), 6.52 (2H, s), 6.69 (1H, dd, J = 7.8, 4.2 Hz), 7.10-7.45 (7H, m), |
| | 7.71 (1H, d, J = 6.1 Hz), 8.28 (1H, s). 5 protons hidden beneath water peak. Rotamers present |
| 13003 | 1.60 (2H, s), 1.92 (5H, s), 3.32 (3H, s), 3.49-3.58 (4H, m), 4.60 (2H, s), 5.66 (1H, s), 5.85- |
| | 5.92 (1H, m), 5.98 (1H, s), 6.50 (2H, s), 6.68 (1H, d, J = 7.8 Hz), 7.17 (1H, d, J = 6.1 Hz), 7.21 |
| | (1H, t, J = 8.0 Hz), 7.34 (1H, d, J = 8.2 Hz), 7.53 (1H, d, J = 7.6 Hz), 7.73 (1H, d, J = 6.1 Hz), |
| | 8.21 (1H, s) |
| 13004 | 1.74 (2H, s), 2.01 (2H, s), 3.28 (3H, s), 3.59 (2H, d, J = 5.9 Hz), 3.67 (2H, s), 4.51 (2H, s), 5.68 |
| | (1H, d, J = 2.8 Hz), 5.89 (1H, dd, J = 7.5, 2.8 Hz), 6.06 (1H, d, J = 6.3 Hz), 6.49 (2H, s), 6.73 |
| | (1H, d, J = 7.7 Hz), 7.15-7.38 (6H, m), 7.51 (2H, dd, J = 7.4, 5.1 Hz), 7.73 (1H, d, J = 5.9 Hz) |
| 13005 | 1.34 (9H, s), 1.57 (2H, s), 1.90 (2H, d, J = 8.1 Hz), 3.26-3.31 (2H, m), 3.34 (3H, s), 3.48- |
| | 3.53 (2H, m), 4.41 (2H, s), 5.69 (1H, d, J = 2.8 Hz), 5.90 (1H, dd, J = 7.5, 2.7 Hz), 5.98 (1H, t), |
| | 6.47 (2H, s), 6.67 (1H, d, J = 7.8 Hz), 7.17 (1H, d, J = 6.4 Hz), 7.20 (1H, d, J = 7.9 Hz), 7.32 |
| | (1H, d, J = 8.4 Hz), 7.53 (1H, d, J = 7.6 Hz), 7.72 (1H, d, J = 6.1 Hz) |
| 13006 | 1.68 (2H, s), 1.92 (2H, s), 3.22 (3H, s), 3.50 (2H, d, J = 6.0 Hz), 3.56 (2H, bs) 4.45 (2H, s), 5.62 |
| | (1H, d, J = 2.7 Hz), 5.84 (1H, dd, J = 7.6, 2.8 Hz), 5.98 (1H, s), 6.41 (2H, s), 6.65 (1H, d, J = 7.6 |
| | Hz), 7.03 (1H, s), 7.09-7.18 (3H, m), 7.27 (3H, t, J = 9.5 Hz), 7.44 (1H, d, J = 7.6 Hz), 7.66 |
| | (1H, d, J = 6.1 Hz), 8.48 (1H, s) |
| 13007 | 1.54 (2H, dd, J = 4.8, 1.9 Hz), 1.82 (2H, d, J = 4.8 Hz), 3.08 (2H, s), 3.32 (3H, s), 3.53 (2H, d, J = |
| | 5.8 Hz), 4.20 (2H, s), 5.80 (1H, d, J = 2.8 Hz), 5.90 (1H, dd, J = 7.5, 2.7 Hz), 6.01 (1H, t, J = |
| | 6.0 Hz), 6.51 (2H, s), 6.65-6.71 (1H, m), 7.12-7.17 (1H, m), 7.20 (1H, t, J = 8.0 Hz), 7.33 |
| | (1H, d, J = 8.3 Hz), 7.55 (1H, d, J = 7.6 Hz), 7.72 (1H, d, J = 6.1 Hz), 8.27 (1H, s) |
| 13008 | 1.46-1.55 (2H, m), 1.82-1.92 (2H, m), 2.48 (2H, t, J = 8.3 Hz), 2.79 (2H, t, J = 7.7 Hz), 3.31 |
| | (3H, s), 3.42 (2H, s), 3.47 (2H, d, J = 5.9 Hz), 4.61 (2H, s), 5.67 (1H, d, J = 2.7 Hz), 5.88 (1H, |
| | dd, J = 7.5, 2.8 Hz), 5.93 (1H, t, J = 6.0 Hz), 6.48 (2H, s), 6.63 (1H, d, J = 7.8 Hz), 7.11-7.28 |
| | (7H, m), 7.33 (1H, d, J = 8.3 Hz), 7.53 (1H, d, J = 7.6 Hz), 7.72 (1H, d, J = 6.1 Hz) |
| 13009 | 1.53-1.61 (2H, m), 1.83-1.90 (2H, m), 3.26 (3H, s), 3.40 (2H, s), 3.47 (2H, d, J = 5.8 Hz), |
| | 4.42 (2H, s), 5.00 (2H, s), 5.60 (1H, s), 5.80 (1H, d, J = 7.5 Hz), 5.94 (1H, t, J = 6.0 Hz), 6.43 |
| | (2H, s), 6.63 (1H, d, J = 7.6 Hz), 7.11 (1H, dd, J = 6.4, 0.8 Hz), 7.15 (1H, t, J = 8.0 Hz), 7.24- |
| | 7.34 (2H, m), 7.45 (1H, d, J = 7.6 Hz), 7.64-7.72 (2H, m), 8.45 (1H, d, J = 4.8 Hz), 8.49 (1H, s) |
| 13010 | 1.49-1.55 (2H, m), 1.87-1.92 (2H, m), 2.54 (2H, t, J = 7.6 Hz), 2.81 (2H, t, J = 7.5 Hz), 3.32 |
| | (3H, s), 3.45 (2H, s), 3.48 (2H, d, J = 5.9 Hz), 4.61 (2H, s), 5.67 (1H, d, J = 2.7 Hz), 5.88 (1H, |
| | dd, J = 7.5, 2.8 Hz), 5.94 (1H, t, J = 5.9 Hz), 6.48 (2H, s), 6.64 (1H, d, J = 7.8 Hz), 7.14 (1H, d, J = |
| | 6.2 Hz), 7.20 (1H, t, J = 8.0 Hz), 7.26 (1H, dd, J = 7.8, 4.8 Hz), 7.33 (1H, d, J = 8.3 Hz), 7.53 |
| | (1H, d, J = 7.5 Hz), 7.64 (1H, d, J = 7.8 Hz), 7.72 (1H, d, J = 6.1 Hz), 8.37 (1H, dd, J = 4.8, 1.7 |
| | Hz), 8.45 (1H, d, J = 2.3 Hz) |
| 13011 | 1.61-1.66 (2H, m), 1.78-1.84 (2H, m), 2.76 (3H, s), 3.32 (3H, s), 3.35 (2H, s), 3.49 (2H, d, J = |
| | 5.8 Hz), 4.44 (2H, s), 4.50 (2H, s), 5.73 (1H, d, J = 2.7 Hz), 5.90 (1H, dd, J = 7.5, 2.8 Hz), |
| | 5.94 (1H, t, J = 6.0 Hz), 6.47 (2H, s), 6.66 (1H, d, J = 7.8 Hz), 7.15-7.34 (8H, m), 7.54 (1H, d, |
| | J = 7.5 Hz), 7.72 (1H, d, J = 6.1 Hz) |
| 13012 | 1.55 (2H, dd, J = 4.4, 1.8 Hz), 1.85 (2H, d, J = 4.5 Hz), 3.31 (3H, s), 3.40 (2H, s), 3.52 (2H, d, J = |
| | 5.8 Hz), 4.20 (2H, d, J = 5.9 Hz), 4.58 (2H, s), 5.66 (1H, d, J = 2.7 Hz), 5.88 (1H, dd, J = 7.5, |
| | 2.8 Hz), 5.95 (1H, t, J = 5.9 Hz), 6.47 (2H, s), 6.67 (1H, d, J = 7.8 Hz), 6.96 (1H, t, J = 6.0 Hz), |
| | 7.16-7.36 (8H, m), 7.52 (1H, d, J = 7.5 Hz), 7.72 (1H, d, J = 6.1 Hz) |
| 13013 | 1.65 (2H, dd, J = 4.6, 1.8 Hz), 1.91-2.05 (2H, m), 3.32 (3H, s), 3.51 (2H, s), 3.55 (2H, d, J = |
| | 6.0 Hz), 4.50 (2H, s), 5.08 (2H, s), 5.69 (1H, d, J = 2.8 Hz), 5.88 (1H, dd, J = 7.5, 2.8 Hz), 6.01 |
| | (1H, t, J = 6.1 Hz), 6.48 (2H, s), 6.70 (1H, d, J = 7.7 Hz), 7.15-7.25 (2H, m), 7.28-7.41 (3H, |
| | m), 7.51 (1H, d, J = 7.5 Hz), 7.70-7.79 (2H, m), 8.52 (1H, ddd, J = 4.8, 1.8, 0.9 Hz) |
| 13014 | 1.72 (2H, dd, J = 4.7, 1.9 Hz), 1.88-1.95 (2H, m), 2.97-3.04 (2H, m), 3.32 (3H, s), 3.40- |
| | 3.47 (2H, m), 3.48-3.54 (4H, m), 4.35 (2H, s), 5.75 (1H, d, J = 2.8 Hz), 5.78 (1H, dd, J = 7.5, |
| | 2.7 Hz), 6.01 (1H, t, J = 6.0 Hz), 6.49 (2H, s), 6.69 (1H, d, J = 7.7 Hz), 7.15-7.36 (8H, m), 7.55 |
| | (1H, d, J = 7.5 Hz), 7.73 (1H, d, J = 6.1 Hz) |
| 13015 | 1.62 (4H, s), 1.67-1.73 (2H, m), 2.53 (2H, s), 2.60-2.64 (2H, m), 2.77 (2H, s), 3.32 (3H, s), |
| | 3.44 (2H, d, J = 5.6 Hz), 4.06 (2H, s), 5.78 (1H, d, J = 2.7 Hz), 5.85-5.94 (2H, m), 6.48 (2H, s), |
| | 6.64 (1H, d, J = 7.8 Hz), 7.11-7.26 (7H, m), 7.31 (1H, d, J = 8.3 Hz), 7.53 (1H, d, J = 7.6 Hz), |
| | 7.71 (1H, d, J = 6.1 Hz) |
| 13016 | 1.49-1.58 (2H, m), 1.85-1.95 (2H, m), 2.60-2.66 (2H, m), 2.95 (2H, t, J = 7.5 Hz), 3.27- |
| | 3.31 (2H, m), 3.47-3.56 (5H, m), 4.59 (2H, s), 5.65 (1H, d, J = 2.7 Hz), 5.87 (1H, dd, J = 7.5, |
| | 2.7 Hz), 5.96 (1H, t, J = 6.0 Hz), 6.48 (2H, s), 6.66 (1H, d, J = 7.7 Hz), 7.13 (1H, ddd, J = 7.6, |
| | 4.9, 1.2 Hz), 7.14-7.17 (1H, m), 7.20 (1H, t, J = 8.0 Hz), 7.25 (1H, d, J = 7.8 Hz), 7.33 (1H, d, |
| | J = 8.3 Hz), 7.52 (1H, d, J = 7.6 Hz), 7.65 (1H, t), 7.73 (1H, d, J = 6.1 Hz), 8.39 (1H, ddd, J = |
| | 4.9, 1.9, 1.0 Hz) |
| 13017 | 1.49-1.54 (2H, m), 1.89 (2H, d, J = 4.5 Hz), 2.90 (2H, t, J = 7.8 Hz), 3.32 (3H, s), 3.45 (2H, s), |
| | 3.48 (2H, d, J = 5.8 Hz), 4.61 (2H, s), 5.67 (1H, d, J = 2.8 Hz), 5.88 (1H, dd, J = 7.6, 2.7 Hz), |
| | 5.94 (1H, t, J = 6.0 Hz), 6.47 (2H, d, J = 5.5 Hz), 6.64 (1H, d, J = 7.7 Hz), 7.12-7.21 (3H, m), |
| | 7.25 (1H, t, J = 7.4 Hz), 7.33 (3H, ddd, J = 8.1, 4.5, 3.1 Hz), 7.53 (1H, d, J = 7.6 Hz), 7.72 (1H, |
| | d, J = 6.1 Hz). 2H under DMSO peak. |
| 13018 | 1.74 (2H, dd, J = 4.6, 1.8 Hz), 1.97 (2H, d, J = 5.5 Hz), 3.28 (3H, s), 3.54 (2H, d, J = 5.9 Hz), |
| | 3.68 (2H, s), 4.76 (2H, s), 5.80 (1H, d, J = 2.7 Hz), 5.86 (1H, dd, J = 7.5, 2.7 Hz), 5.99 (1H, t, J = |
| | 6.0 Hz), 6.43 (2H, s), 6.67 (1H, d, J = 7.7 Hz), 6.97 (1H, td, J = 7.7, 1.3 Hz), 7.08 (1H, td, J = |
| | 7.7, 1.2 Hz), 7.12-7.20 (2H, m), 7.23-7.33 (3H, m), 7.49 (1H, d, J = 7.5 Hz), 7.68 (1H, d, J = |
| | 6.1 Hz) |
| 13019 | 1.61-1.68 (2H, m), 1.86-1.92 (2H, m), 3.31 (3H, s), 3.53 (2H, d, J = 5.6 Hz), 3.57 (2H, s), |
| | 4.63 (2H, s), 5.71 (1H, d, J = 2.7 Hz), 5.89 (1H, dd, J = 7.5, 2.8 Hz), 5.96 (1H, t, J = 5.8 Hz), |
| | 6.48 (2H, s), 6.68 (1H, d, J = 7.7 Hz), 6.91 (1H, t, J = 7.3 Hz), 7.17-7.24 (4H, m), 7.34 (1H, d, |
| | J = 8.3 Hz), 7.47 (2H, dd, J = 8.6, 1.3 Hz), 7.53 (1H, d, J = 7.5 Hz), 7.72 (1H, d, J = 6.1 Hz), 8.56 |
| | (1H, s) |
| 13020 | 1.62 (2H, dd, J = 4.6, 1.7 Hz), 1.90-1.95 (2H, m), 3.47 (3H, s), 3.53 (2H, d, J = 5.8 Hz), 4.45 |
| | (2H, s), 5.08 (2H, s), 5.62 (1H, s), 5.84 (1H, d, J = 7.6 Hz), 5.99 (1H, t, J = 6.0 Hz), 6.47 (2H, s), |
| | 6.68 (1H, d, J = 7.7 Hz), 7.13-7.24 (2H, m), 7.28-7.36 (3H, m), 7.43 (2H, d, J = 7.2 Hz), 7.49 |
| | (1H, d, J = 7.5 Hz), 7.72 (1H, d, J = 6.1 Hz) |
| 13021 | 1.59 (d, J = 3.6 Hz, 2H), 1.90-1.97 (m, 2H), 3.30 (s, 3H), 3.53 (d, J = 5.8 Hz, 2H), 3.58 (s, |
| | 2H), 3.63 (s, 2H), 4.59 (s, 2H), 5.64 (d, J = 2.8 Hz, 1H), 5.86 (dd, J = 7.5, 2.7 Hz, 1H), 5.98 (t, J = |
| | 6.0 Hz, 1H), 6.48 (d, J = 4.4 Hz, 2H), 6.68 (d, J = 7.7 Hz, 1H), 7.14-7.17 (m, 1H), 7.18- |
| | 7.25 (m, 4H), 7.25-7.30 (m, 2H), 7.34 (d, J = 8.3 Hz, 1H), 7.51 (d, J = 7.6 Hz, 1H), 7.73 (d, J = |
| | 6.1 Hz, 1H) |
| 13022 | 1.57 (2H, dd, J = 4.5, 1.8 Hz), 1.82-1.95 (2H, m), 3.32 (3H, s), 3.37 (2H, s), 3.50 (2H, d, J = |
| | 5.8 Hz), 3.78 (3H, s), 4.46 (2H, s), 4.84 (2H, s), 5.66 (1H, s), 5.81-5.90 (1H, m), 5.98 (1H, t, J = |
| | 6.1 Hz), 6.46 (2H, d, J = 5.5 Hz), 6.66 (1H, d, J = 7.7 Hz), 7.15 (1H, d, J = 6.1 Hz), 7.19 (1H, t, |
| | J = 8.0 Hz), 7.32 (1H, d, J = 8.3 Hz), 7.36 (1H, s), 7.52 (1H, d, J = 7.6 Hz), 7.64 (1H, s), 7.71 |
| | (1H, d, J = 6.1 Hz) |

### Biological Methods

### Determination of FXIIa Inhibition

In vitro inhibition of Factor XIIa was determined using an IC₅₀ assay based on standard literature methods (see e.g Baeriswyl et al., ACS Chem. Biol., 2015, 10 (8) 1861; Bouckaert et al., European Journal of Medicinal Chemistry 110 (2016) 181). Human Factor XIIa (Enzyme Research Laboratories) was incubated at 25° C. with the fluorogenic substrate H-DPro-Phe-Arg-AFC (Peptide Protein Science) and various concentrations of the test compound. Protease activity was measured by monitoring the accumulation of liberated fluorescence from the substrate over 5 min at 25° C. The linear rate of fluorescence increase per minute was expressed as percentage (%) activity. The Km for the cleavage of the substrate by FXIIa was determined by standard transformation of the Michaelis-Menten equation. The compound inhibitor assays were performed at substrate Km concentration. IC₅₀ values were calculated as the concentration of inhibitor giving 50% inhibition (IC₅₀) of the uninhibited enzyme activity (100%). Data acquired from this assay are shown in Table 16 below using the following scale:

| Category | IC₅₀ (nM) |
|---|---|
| AA | 1-10 |
| A | 10-100 |
| B | 100-300 |
| C | 300-1,000 |
| D | 1,000-3,000 |
| E | 3,000-10,000 |
| F | 10,000-40,000 |
| G | 40,000-100,000 |

For the test compounds that did not achieve 50% inhibition the following scale is used:

| Category | |
|---|---|
| H1 | >20% inhibition @ 40 μM |
| H2 | >20% inhibition @ 100 μM |
| H3 | >20% inhibition @ 400 μM |

TABLE 16
Human FXIIa data, molecular weight and LCMS data

| Example | Human FXII | Molecular | LCMS |
|---|---|---|---|
| No | IC50 (nm) | weight | Mass Ion |
| 1001 | B | 385.5 | 386.2 |
| 1002 | B | 389.5 | 390.2 |
| 1002.1 | B | 389.5 | 390.2 |
| 1002.2 | C | 389.5 | 390.2 |
| 1004 | E | 374.5 | 375.2 |
| 1005.1 | C | 391.6 | 392.2 |
| 1006 | B | 399.5 | 400.2 |
| 1009 | F | 359.4 | 360.3 |
| 1010 | C | 393.9 | 394.3 |
| 1011 | F | 370.5 | 371.3 |
| 1012 | D | 373.5 | 374.3 |
| 1013 | C | 386.5 | 387.2 |
| 1014 | D | 384.5 | 385.2 |
| 1015 | F | 389.5 | 390.2 |
| 1016 | E | 388.5 | 389.2 |
| 1017 | B | 385.5 | 386.2 |
| 1018 | C | 385.5 | 386.2 |
| 1019 | C | 376.5 | 377.2 |
| 1101 | E | 385.5 | 386.2 |
| 1105 | E | 385.5 | 386.2 |
| 1109 | C | 400.5 | 401.2 |
| 1110 | E | 386.5 | 387.1 |
| 1113 | D | 400.5 | 401.2 |
| 1118 | D | 375.5 | 376.2 |
| 1125 | F | 385.5 | 386.2 |
| 1126 | F | 371.5 | 372.2 |
| 1127 | E | 386.5 | 386.2 |
| 1129 | H2 | 393.9 | 394.2/396.2 |
| 1130 | C | 397.9 | 398.2 |
| 1131 | F | 371.4 | 372.1 |
| 1132 | F | 449.5 | 450.1 |
| 1133 | B | 401.3 | 400.5 |
| 1134 | B | 391.3 | 390.5 |
| 1135 | C | 387.3 | 386.5 |
| 2020 | A | 401.5 | 402.2 |
| 2022 | B | 401.5 | 402.2 |
| 2204 | C | 453.4 | 454.4 |
| 2205 | C | 392.5 | 393.2 |
| 2206 | D | 470.5 | 471.2 |
| 2207 | D | 402.5 | 403.1 |
| 2208 | E | 388.4 | 389.2 |
| 2210 | D | 402.5 | 403.2 |
| 2211 | B | 430.5 | 431.2 |
| 2212 | A | 415.5 | 416.4 |
| 4401 | C | 373.5 | 374.2 |
| 5003 | B | 373.5 | 374.3 |
| 6601 | A | 401.5 | 402.2 |
| 6602 | A | 401.5 | 402.2 |
| 6605 | E | 389.5 | 390.3 |
| 6606 | D | 409.9 | 410.1 |
| 6608 | C | 453.4 | 454.3 |
| 6609 | B | 392.5 | 393.2 |
| 6610 | C | 405.5 | 406.2 |
| 6611 | D | 409.9 | 410.1 |
| 6612 | D | 402.5 | 403.2 |
| 6613 | C | 451.5 | 452.1 |
| 6614 | B | 402.5 | 403.2 |
| 6615 | E | 470.5 | 471.4 |
| 6616 | C | 402.5 | 403.4 |
| 6617 | A | 415.5 | 416.4 |
| 6618 | A | 416.5 | 417.4 |
| 6619 | C | 401.5 | 402.3 |
| 6620 | C | 452.5 | 453.4 |
| 6621 | B | 430.5 | 431.4 |
| 6622 | A | 415.5 | 416.4 |
| 6623 | E | 402.5 | 403.4 |
| 6624 | A | 415.5 | 416.4 |
| 6625 | C | 419.5 | 420.4 |
| 6626 | A | 415.5 | 416.3 |
| 6627 | D | 469.5 | 470.3 |
| 6628 | B | 430.5 | 431.4 |
| 6629 | A | 402.5 | 403.4 |
| 6630 | B | 469.5 | 470.4 |
| 6631 | B | 431.5 | 432.4 |
| 6633 | B | 415.5 | 416.4 |
| 6634 | D | 402.5 | 403.4 |
| 6635 | F | 430.5 | 431.5 |
| 6636 | E | 430.5 | 431.4 |
| 6637 | B | 430.5 | 431.4 |
| 6638 | D | 403.4 | 404.3 |
| 6639 | A | 441.5 | 442.3 |
| 6640 | B | 444.5 | 445.3 |
| 6641 | B | 443.5 | 444.4 |
| 6642 | B | 416.5 | 417.4 |
| 6643 | C | 442.5 | 443.3 |
| 6644 | C | 451.5 | 452.2 |
| 6645 | F | 335.4 | 336.2 |
| 6646 | G | 335.4 | 336.2 |
| 6647 | G | 336.4 | 337.2 |
| 6648 | F | 336.4 | 337.3 |
| 6649 | F | 352.4 | 353.3 |
| 6650 | B | 444.5 | 445.3 |
| 6651 | H1 | 424.9 | 425.1 |
| 6653 | C | 444.5 | 445.3 |
| 6654 | F | 444.5 | 445.3 |
| 6656 | H2 | 416.5 | 417.5 |
| 6658 | F | 374.4 | 375.3 |
| 6659 | G | 431.5 | 432.4 |
| 6660 | E | 365.4 | 366.1 |
| 6661 | F | 350.4 | 351.3 |
| 6663 | H2 | 380.4 | 381.4 |
| 7702 | C | 430.5 | 431.2 |
| 7703 | B | 429.5 | 430.2 |
| 7704 | C | 430.5 | 431.2 |
| 8801 | F | 385.5 | 386.4 |
| 8803 | D | 399.5 | 400.3 |
| 8804 | D | 399.5 | 400.6 |
| 8805 | D | 399.5 | 400.4 |
| 8806 | B | 399.5 | 400.2 |
| 8807 | C | 399.5 | 400.2 |
| 8808 | F | 385.5 | 386.2 |
| 9001 | F | 385.5 | 386.1 |
| 9002 | F | 385.5 | 386.6 |
| 9003 | F | 385.5 | 386.6 |
| 10901 | A | 415.5 | 416.2 |
| 10903 | C | 419.5 | 420.4 |
| 10904 | A | 415.5 | 416.2 |
| 10906 | B | 406.5 | 407.2 |
| 10907 | D | 403.5 | 404.2 |
| 10908 | C | 419.5 | 420.2 |
| 10909 | D | 423.9 | 424.1 |
| 10910 | B | 467.5 | 468.2 |
| 10911 | E | 406.5 | 407.2 |
| 10912 | F | 377.4 | 378.2 |
| 10914 | E | 376.5 | 377.2 |
| 10915 | F | 376.5 | 377.2 |
| 10918 | H3 | 383.9 | 384.3 |
| 10919 | H3 | 383.9 | 384.3 |
| 10920 | B | 429.5 | 430.4 |
| 10921 | G | 434.9 | 435.3 |
| 10922 | C | 449.9 | 450.6 |
| 10923 | B | 429.5 | 430.4 |
| 10924 | B | 449.9 | 450.3 |
| 10925 | A | 429.5 | 430.4 |
| 10926 | A | 416.5 | 417.2 |
| 10928 | A | 420.5 | 421.2 |
| 10929 | C | 416.5 | 417.4 |
| 10930 | A | 429.5 | 430.4 |
| 10931 | A | 430.5 | 431.4 |
| 10932 | B | 483.5 | 484.4 |
| 10933 | D | 440.9 | 441.0 |
| 10934 | C | 434.5 | 435.4 |
| 10936 | E | 366.4 | 367.3 |
| 10937 | E | 377.4 | 378.4 |
| 10938 | C | 444.5 | 445.4 |
| 10939 | C | 458.6 | 459.4 |
| 10940 | B | 407.5 | 408.3 |
| 10941 | C | 430.5 | 431.4 |
| 10942 | B | 413.5 | 414.3 |
| 10943 | D | 406.5 | 407.4 |
| 10944 | D | 406.5 | 407.4 |
| 10945 | E | 406.5 | 407.4 |
| 10946 | E | 380.4 | 381.4 |
| 10948 | D | 419.5 | 420.4 |
| 10949 | C | 448.6 | 449.4 |
| 10951 | C | 474.5 | 475.3 |
| 10952 | C | 392.5 | 393.2 |
| 10953 | B | 390.5 | 391.3 |
| 10954 | E | 406.5 | 407.2 |
| 10955 | D | 390.5 | 391.2 |
| 10956 | D | 390.5 | 391.3 |
| 10957 | E | 390.5 | 391.3 |
| 10958 | F | 400.5 | 401.5 |
| 10961 | C | 484.5 | 485.2 |
| 10962 | D | 421.5 | 422.2 |
| 10963 | E | 377.4 | 378.2 |
| 10964 | A | 434.5 | 435.2 |
| 10965 | H1 | 377.4 | 378.2 |
| 10966 | H1 | 377.4 | 378.2 |
| 10968 | E | 376.5 | 377.2 |
| 10969 | D | 416.5 | 417.2 |
| 10970 | H2 | 422.9 | 423.3 |
| 10971 | C | 391.5 | 392.2 |
| 10972 | A | 432.5 | 433.2 |
| 10973 | D | 416.5 | 417.2 |
| 10974 | E | 391.5 | 392.2 |
| 10975 | C | 442.5 | 443.2 |
| 10976 | F | 377.4 | 378.2 |
| 11001 | B | 406.5 | 407.4 |
| 12001 | A | 525.6 | 526.3 |
| 12007 | A | 391.5 | 392.2 |
| 12008 | A | 511.6 | 512.2 |
| 12009 | A | 433.5 | 434.4 |
| 12010 | B | 525.6 | 526.5 |
| 12011 | C | 391.5 | 392.4 |
| 12012 | B | 563.7 | 564.6 |
| 12013 | C | 449.5 | 450.4 |
| 12014 | A | 559.7 | 560.2 |
| 12015 | A | 546.0 | 547.3 |
| 12016 | A | 523.6 | 524.4 |
| 12014 | A | 524.6 | 252.4 |
| 12018 | A | 563.7 | 564.6 |
| 12019 | A | 509.6 | 510.4 |
| 12020 | B | 561.6 | 562.3 |
| 12021 | A | 526.6 | 527.6 |
| 12022 | A | 526.6 | 527.4 |
| 12023 | A | 560.0 | 560.3 |
| 12024 | B | 575.7 | 576.7 |
| 12025 | D | 534.0 | 534.2 |
| 12026 | B | 529.6 | 530.4 |
| 12027 | B | 491.6 | 492.4 |
| 12028 | A | 509.6 | 510.1 |
| 12031 | B | 545.6 | 546.4 |
| 12034 | A | 549.6 | 550.3 |
| 12035 | A | 495.6 | 496.3 |
| 12036 | AA | 510.6 | 510.6 |
| 12038 | AA | 538.6 | 539.6 |
| 12039 | A | 523.6 | 524.5 |
| 12040 | A | 548.6 | 549.4 |
| 12041 | AA | 538.6 | 539.4 |
| 12042 | A | 553.7 | 554.4 |
| 12044 | A | 548.6 | 549.7 |
| 12045 | F | 476.6 | 477.2 |
| 12046 | H2 | 490.6 | 491.3 |
| 12047 | A | 574.7 | 575.4 |
| 12048 | A | 573.6 | 574.7 |
| 12049 | A | 558.1 | 558.9 |
| 12050 | A | 591.6 | 592.7 |
| 12051 | A | 567.6 | 566.4 |
| 12052 | A | 574.7 | 575.3 |
| 12053 | A | 602.5 | 604.1 |
| 12054 | A | 540.6 | 541.3 |
| 12055 | A | 574.7 | 575.1 |
| 12056 | A | 589.6 | 590.6 |
| 12057 | AA | 508.6 | 509.2 |
| 12058 | A | 542.6 | 543.7 |
| 12059 | A | 540.6 | 541.7 |
| 12060 | AA | 545.6 | 546.3 |
| 12061 | AA | 540.6 | 541.3 |
| 12062 | A | 554.6 | 555.3 |
| 12063 | H2 | 476.6 | 477.3 |
| 12064 | A | 468.6 | 469.2 |
| 12065 | AA | 468.6 | 469.2 |
| 12067 | A | 554.6 | 555.3 |
| 12068 | AA | 528.6 | 529.6 |
| 12069 | A | 592.6 | 593.3 |
| 12070 | A | 564.6 | 565.4 |
| 12071 | A | 525.6 | 526.3 |
| 12072 | C | 542.6 | 543.3 |
| 13001 | A | 525.6 | 526.3 |
| 13002 | F | 606.6 | 607.2 |
| 13003 | B | 433.5 | 434.3 |
| 13004 | B | 546.0 | 546.3 |
| 13005 | B | 491.6 | 492.4 |
| 13006 | A | 511.6 | 512.3 |
| 13007 | B | 391.5 | 392.3 |
| 13008 | C | 523.6 | 524.3 |
| 13009 | A | 526.6 | 527.3 |
| 13010 | B | 524.6 | 525.3 |
| 13011 | C | 538.6 | 539.3 |
| 13012 | A | 524.6 | 525.4 |
| 13013 | B | 526.6 | 527.3 |
| 13014 | B | 559.7 | 560.2 |
| 13015 | B | 509.6 | 510.3 |
| 13016 | C | 524.6 | 525.3 |
| 13017 | C | 558.1 | 558.2 |
| 13018 | C | 508.6 | 509.3 |
| 13019 | C | 510.6 | 511.3 |
| 13020 | A | 560.0 | 561.3 |
| 13021 | C | 509.6 | 510.2 |
| 13022 | A | 529.6 | 530.3 |

### Determination of Related Protease Inhibition

In vitro inhibition of related proteases was determined using an IC₅₀ assay based on standard literature methods (see e.g. Shori et al., Biochem. Pharmacol., 1992, 43, 1209; Bouckaert et al., European Journal of Medicinal Chemistry 110 (2016) 181). Human serine protease enzyme Plasma Kallikrein, KLK1, FXa, Plasmin, Thrombin and Trypsin were assayed for enzymatic activity using an appropriate fluorogenic substrate at Km concentration, FXIa at fixed substrate concentration of 100 μM and various concentrations of the test compound. Protease activity was measured by monitoring the accumulation of liberated fluorescence from the substrate over 5 min at 25° C. The linear rate of fluorescence increase per minute was expressed as percentage (%) activity. IC₅₀ values were calculated as the concentration of inhibitor giving 50% inhibition of the uninhibited enzyme activity (100%).

Data acquired from this assay are shown in Table 17 using the scale shown in Table 18.

TABLE 17
Enzyme selectivity data

| | IC₅₀ | IC₅₀ | IC₅₀ | IC₅₀ | IC₅₀ | IC₅₀ | IC₅₀ |
|---|---|---|---|---|---|---|---|
| Example | (human | (human | (human | (human | (human | (human | (human |
| No | PKal) nM | KLK1) nM | plasmin) nM | FXa) nM | FXIa) nM | thrombin) nM | trypsin) nM |
| 1001 | G3 | E | G3 | F | G3 | F | G3 |
| 1002 | G3 | E | G3 | F | G3 | F | F |
| 1002.1 | G3 | E | | F | | E | |
| 1002.2 | G3 | | | | | | |
| 1004 | G3 | E | | D | | F | |
| 1005.1 | G3 | | | | | | |
| 1006 | G3 | D | | D | | F | |
| 1009 | G4 | G4 | | | | | |
| 1010 | | G3 | | D | | | |
| 1011 | G4 | F | | D | | F | |
| 1012 | G4 | G3 | | D | | D | |
| 1013 | G3 | | | | | | |
| 1014 | G3 | | | | | | |
| 1015 | G3 | | | | | | |
| 1016 | G3 | E | | G3 | | G3 | |
| 1017 | G3 | E | | F | | F | |
| 1018 | G3 | | | | | | |
| 1019 | G3 | E | | F | | F | |
| 1101 | G3 | E | | D | | F | |
| 1105 | G3 | E | | D | | F | |
| 1109 | G3 | D | | F | | F | |
| 1113 | G3 | | | | | | |
| 1118 | G3 | E | | G3 | | F | |
| 1125 | G3 | | | | | | |
| 1126 | G3 | | | | | | |
| 1127 | G3 | | | | | | |
| 1129 | G4 | | | | | | |
| 1130 | G3 | | | | | | |
| 1131 | G3 | | | | | | |
| 2020 | G3 | E | | G3 | | G3 | |
| 2022 | G3 | E | | G3 | | G3 | |
| 2204 | G3 | E | | G3 | | G3 | |
| 2205 | G3 | F | G3 | G3 | | G3 | G3 |
| 2206 | G3 | | | | | | |
| 2207 | G3 | | | | | | |
| 2208 | G3 | | | | | | |
| 2210 | G3 | | | | | | |
| 2211 | G1 | D | | G3 | | | |
| 2212 | F | D | G3 | F | | G3 | F |
| 4401 | G3 | D | | G3 | | G3 | |
| 5003 | G3 | E | | G3 | | G3 | |
| 6601 | G3 | E | | F | | F | |
| 6602 | G3 | E | G3 | F | | F | G3 |
| 6605 | G3 | F | | F | | F | |
| 6606 | G3 | | | | | | |
| 6608 | G3 | E | | G3 | | G3 | |
| 6609 | G3 | E | | G3 | | G3 | |
| 6610 | G3 | | | | | | |
| 6611 | G3 | | | | | | |
| 6612 | G3 | | | | | | |
| 6613 | | E | | F | | | |
| 6614 | G1 | E | | G3 | | | |
| 6615 | | E | | G3 | | | |
| 6616 | | E | | G3 | | | |
| 6617 | G3 | E | | F | | | |
| 6618 | G3 | E | G3 | F | | G3 | F |
| 6619 | | F | | | | | |
| 6620 | G3 | E | G3 | G3 | | G3 | E |
| 6621 | G3 | E | G3 | G3 | | G3 | G3 |
| 6622 | | D | | | | | |
| 6624 | F | D | G3 | D | | G3 | F |
| 6626 | | D | | | | | |
| 6627 | | E | | | | | |
| 6628 | | E | | | | | |
| 6629 | G3 | D | F | G3 | | G3 | F |
| 7703 | G3 | | | | | | |
| 7704 | G3 | | | | | | |
| 8801 | G3 | | | | | | |
| 8803 | G3 | | | | | | |
| 8804 | G3 | | | | | | |
| 8805 | G3 | | | | | | |
| 8806 | G3 | F | G3 | G3 | | G3 | G3 |
| 8807 | | F | | | | | |
| 8808 | G3 | | | | | | |
| 9001 | G3 | | | | | | |
| 9002 | G3 | | | | | | |
| 9003 | G3 | | | | | | |
| 10901 | G3 | E | | F | | F | |
| 10903 | G1 | | | | | | |
| 10904 | G3 | E | | G3 | | F | |
| 10906 | G3 | F | G3 | G3 | | G3 | F |
| 10907 | G3 | G3 | | G3 | | G3 | |
| 10908 | G3 | | | | | | |
| 10909 | G3 | | | | | | |
| 10910 | G3 | | | | | | |
| 10911 | G3 | | | | | | |
| 10912 | G3 | | | | | | |
| 10914 | G3 | | | | | | |
| 10915 | G3 | | | | | | |
| 10918 | G3 | G4 | | G3 | | | |
| 10919 | G3 | G4 | | G3 | | | |
| 10920 | E | E | | F | | | |
| 10921 | | F | | | | | |
| 10922 | | C | | | | | |
| 10923 | | C | | | | | |
| 10924 | | E | | | | | |
| 10925 | | E | | | | | |
| 10926 | G3 | E | G3 | G3 | | G3 | E |
| 10928 | G3 | F | G3 | F | | G3 | F |
| 10940 | G3 | F | G3 | G3 | | G3 | F |
| 10942 | | E | | G3 | | | |
| 11001 | G3 | F | G3 | G3 | | G3 | F |
| 12001 | G3 | E | | G3 | | G3 | |
| 12007 | G1 | F | | G3 | | | |
| 12008 | G1 | D | | G3 | | | |
| 12009 | G1 | F | | G3 | | | |
| 12010 | | A | | | | | |
| 12017 | F | E | F | F | | G3 | G3 |
| 12019 | G3 | D | G3 | G3 | | G3 | G3 |
| 12036 | F | D | E | F | | G3 | G3 |
| 12038 | E | E | F | D | | G3 | F |
| 13001 | G3 | G1 | | G1 | | | |
| 13002 | G3 | | | | | | |

TABLE 18
Scale used to present enzyme selectivity data

| Category | IC₅₀ (nM) |
|---|---|
| A | 10-100 |
| B | 100-300 |
| C | 300-1,000 |
| D | 1,000-3,000 |
| E | 3,000-10,000 |
| F | 10,000-40,000 |
| G1 | >4,000 |
| G2 | >20,000 |
| G3 | >40,000 |
| G4 | >100,000 |

### Pharmacokinetics

Pharmacokinetic studies of the compounds in Table 19 were performed to assess the pharmacokinetics following a single intravenous dose and a single oral dose in male Sprague-Dawley rats. Two rats were given a single intravenous dose of 1 mL/kg of a nominal 1 mg/ml (1 mg/kg) composition of test compound in 10% DMSO/10% Cremophor EL/80% SWFI vehicle. Two rats were given a single oral dose of 5 ml/kg of a nominal 1 mg/mL (5 mg/kg) composition of test compound in 10% DMSO/10% Cremophor EL/80% SWFI vehicle.

Following intravenous dosing, blood samples were collected over a period of 12 h. Sample times were 2, 5, 15 and 30 min then 1, 2, 4, 6, and 12 h. Following oral dosing, blood samples were collected over a period of 24 h. Sample times were 5, 15 and 30 min then 1, 2, 4, 6, 8, 12 and 24 h.

Following collection, blood samples were centrifuged and the plasma fraction analysed for concentration of test compound by LCMS. Oral bioavailability and half-life calculations from these studies were obtained using Phoenix WinNonlin (v8.0) and are shown below:

TABLE 19
Oral exposure data

| Example | Dose iv | Dose po | | T_{1/2} |
|---|---|---|---|---|
| No | (mg/kg) | (mg/kg) | F % | (min) |
| 1001 | 0.88 | 4.45 | 30.4% | 469.5 |
| 1002.1 | 1.1 | 5.7 | 41.0% | 441.3 |
| 2020 | 1.0 | 5.3 | 19.9% | 632.1 |
| 2022 | 1.06 | 5.3 | 42.6% | 188.0 |
| 6602 | 1.11 | 5.7 | 33.5% | 342.2 |
| 6624 | 1.04 | 5.2 | 14.8% | 398.1 |
| 10901 | 0.95 | 4.8 | 9.3% | 715.3 |
| 10906 | 0.54 | 2.7 | 40.5% | 152.9 |
| 12001 | 1.59 | 8.1 | 8.2% | 68.2 |
| 12016 | 0.48 | 5.2 | 0.4% | 64.8 |

1. A compound of formula (I), 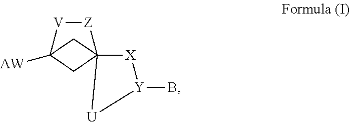
wherein:
U is absent —C(R16)(R17)-, CH₂C(R16)(R17) or C(R16)(R17)CH₂;
-V-Z- is:
absent, —CH₂—, or —CH₂—O—CH₂; or
V is CH₂, O or NR18, and Z is —C(R16)(R17)-CH₂— or —C(R16)(R17)-; or,
V is —CH₂—C(R16)(R17)- or —C(R16)(R17)-, and Z is CH₂, O or NR18;
wherein R18 is —H, alkyl, (CH₂)₀₋₆-aryl, (CH₂)₀₋₆-heteroaryl^{a}, C(═O)R19, C(═O)OR19, C(═O)NHR19, C(═O)N(alkyl)(R19), C(═O)SR19 or SO₂R19;
wherein R19 is alkyl, cycloalkyl, (CH₂)₀₋₆-aryl and or (CH₂)₀₋₆-heteroaryl^{a};
X is a bond, O, CR1R2, C˜O or NR12;
Y is O, CR1R2, CR1, C—O, N or NR12;
R1 is H, alkyl, alkoxy, OH, halo and or NR13R14;
R2 is H or small alkyl;
wherein when one of X or Y is C═O, the other is O, CR1R2, CR1, N or NR12;
wherein when X is NR12, Y is CR1R2, CR1 or C═O;
wherein when Y is NR12 or N, X is a bond, CR1R2 or C═O;
wherein when X is O, Y is CR1R2, CR1 or C═O;
wherein when Y is O, X is a bond, CR1R2 or C═O;
wherein when X is a bond, Y is O, N or NR12;
wherein when U is not absent, -V-Z- is absent;
wherein when -V-Z- is not absent, U is absent;
B is:
(i) heteroaryl^{a};
(ii) aryl;
(iii) a 5- to 6-membered non-aromatic heterocyclic ring containing one N ring member, which is unsaturated with 1 or 2 double bonds, wherein the non-aromatic heterocyclic ring is optionally substituted by 1, 2 or 3 substituents that are, independently, alkyl, alkoxy, aryl^{b}, OH, OCF₃, halo, oxo, CN, or CF₃; or
(iv) a fused 5,5-, 6,5- or 6,6-bicyclic ring containing an aromatic ring fused to a non-aromatic ring, wherein the bicyclic ring optionally contains one or two N ring members, wherein the fused 5,5-, 6,5- or 6,6-bicyclic ring is optionally substituted with 1, 2, or 3 substituted by up to three substituents that are, independently, alkyl, alkoxy, OH, OCF₃, halo, oxo, CN, or CF₃, wherein the 6,5-bicyclic ring is optionally attached via the 6- or 5-membered ring;
AW- is:
—(CH₂)₀₋₆—(CHR15)-(CH₂)₀₋₆-A, —(CHR12)-A, —O—(CHR12)-A, —(CH₂)₀₋₆-A, —(CH₂)₀₋₆—O—(CH₂)₀₋₆-A, —(CH₂)₀₋₆—NH—(CH₂)₀₋₆-A, —(CH₂)₀₋₆—NR12-(CH₂)₁₋₆—C(═O)-A, —(CH₂)₀₋₆—NH—C(═O)—(CH₂)₀₋₆-A, —C(═O)NR12-(CH₂)₀₋₆-A, —(CH₂)₀₋₆—C(═O)—(CH₂)₀₋₆-A, —(CH₂)₀₋₆-(phenyl)-(CH₂)₀₋₆-A, —NH—SO₂-A or —SO₂—NH-A;
A is a 4- to 15-membered mono-, bi-, or tri-cyclic ring system, containing one N ring member and optionally one, two or three further ring members that are, independently, N, O or S, optionally wherein the ring system is substituted, with 1, 2, 3 or 4 substituents that are, independently, halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, or CN;
wherein when A is a bicyclic ring system, the bicyclic ring system is fused, bridged or spiro;
wherein when A is a tricyclic ring system, each of the three rings in the tricyclic ring system is either fused, bridged or spiro to at least one of the other rings in the tricyclic ring system;
wherein when -V-Z- is —CH₂—, U is absent, and AW- is A-(C═O)—, A is not substituted by —(CH₂)₀-heteroaryl;
alkyl is a linear saturated hydrocarbon having up to 10 carbon atoms (C₁-C₁₀) or a branched saturated hydrocarbon of between 3 and 10 carbon atoms (C₃-C₁₀); alkyl is optionally substituted with 1, 2 or 3 substituents that are, independently, (C₁-C₆)alkoxy, OH, —NR13R14, —C(═O)OR13, —C(═O)NR13R14, CN, CF₃, or halo;
alkyl^{b} is a linear saturated hydrocarbon having up to 10 carbon atoms (C₁-C₁₀) or a branched saturated hydrocarbon of between 3 and 10 carbon atoms (C₃-C₁₀); alkyl^{b} is optionally substituted with 1, 2 or 3 substituents that are, independently, (C₁-C₆)alkoxy, OH, CN, CF₃, or halo;
small alkyl is a linear saturated hydrocarbon having up to 4 carbon atoms (C₁-C₄) or a branched saturated hydrocarbon of between 3 and 4 carbon atoms (C₃-C₄); small alkyl is optionally substituted with 1 or 2 substituents that are, independently, (C₁-C₆)alkoxy, OH, NR13R14, C(═O)OR13, C(═O)NR13R14, CN, CF₃, or halo;
aryl is phenyl, biphenyl or naphthyl; aryl is optionally substituted with 1, 2 or 3 substituents that are, independently, alkyl, alkoxy, methylenedioxy, ethylenedioxy, OH, halo, CN, —(CH₂)₀₋₃—O-heteroaryl^{a}, aryl^{b}, —O-aryl^{b}, —(CH₂)₁₋₃-aryl^{b}, —(CH₂)₀₋₃-heteroaryl^{a}, —C(═O)OR13, —C(═O)NR13R14, —(CH₂)₀₋₃—NR13R14, OCF₃ and or CF₃;
aryl^{b} is phenyl, biphenyl or naphthyl; aryl^{b} is optionally substituted with 1, 2 or 3 substituents that are, independently, alkyl^{b}, alkoxy, OH, halo, CN, or CF₃;
cycloalkyl is a monocyclic saturated hydrocarbon ring of between 3 and 6 carbon atoms (C₃-C₆); cycloalkyl is optionally substituted with 1 or 2 substituents that are, independently, alkyl, (C₁-C₆)alkoxy, OH, CN, CF₃, or halo;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy is optionally substituted with 1 or 2 substituents that are, independently, OH, CN, CF₃, or fluoro;
halo is F, Cl, Br, or I;
heteroaryl is a 5- or 6-membered carbon-containing aromatic ring containing one, two or three ring members that are N, NR8, S, or O; heteroaryl is optionally substituted with 1, 2 or 3 substituents that are, independently, alkyl, alkoxy, OH, OCF₃, halo, CN, or CF₃;
heteroaryl^{a} is a 5, 6, 9 or 10 membered mono- or bi-cyclic aromatic ring, containing 1, 2, 3 or 4 ring members that are, independently, N, NR12, S or O; heteroaryl^{a} is optionally substituted with 1, 2 or 3 substituents that are, independently, alkyl, alkoxy, OH, OCF₃, halo, CN, aryl^{b}, —(CH₂)₀₋₃-NR13R14, heteroaryl^{b}, —C(═O)OR12, —C(═O)NR13R14 or CF₃;
heteroaryl^{b} is a 5, 6, 9 or 10 membered mono- or bi-cyclic aromatic ring, containing 1, 2 or 3 ring members that are, independently, N, NR 12, S or O; wherein heteroaryl^{b} is optionally substituted with 1, 2 or 3 substituents that are, independently, alkyl^{b}, alkoxy, OH, halo, CN, aryl^{b}, —(CH₂)₁₋₃-aryl^{b}, or CF₃;
R8 is independently H, alkyl, cycloalkyl, or heterocycloalkyl;
heterocycloalkyl is a non-aromatic carbon-containing monocyclic ring containing 3, 4, 5, or 6, ring members, wherein at least one ring member is independently N, NR12, S, or O; heterocycloalkyl is optionally be substituted with 1 or 2 substituents that are, independently, alkyl, (C₁-C₆)alkoxy, OH, CN, CF₃, or halo;
R12 is independently H, alkyl, or cycloalkyl;
R13 and R14 are independently H, alkyl^{b}, aryl^{b} or heteroaryl^{b} or R13 and R14 together with the nitrogen atom to which they are attached form a carbon-containing 4-, 5-, 6- or 7-membered heterocyclic ring, optionally containing an additional heteroatom that is N, NR12, S, SO, SO₂, or O, which is saturated or unsaturated with 1 or 2 double bonds and is optionally mono- or di-substituted with substituents that are oxo, alkyl^{b}, alkoxy, OH, halo or CF₃;
R15 is alkyl, halo, CF₃, CN, OH, alkoxy, NR13R14, or CONR13R14;
R16 and R17 are independently H or small alkyl;
or a tautomer, isomer, stereoisome, deuterated isotope, or pharmaceutically acceptable salt and/or solvate thereof;
wherein the compound is not N-(2-chlorophenyl)-3-((5-cyano-1H-indazol-1-yl)-methyl)-N-methylbicyclo-[1.1.1]pentane-1-carboxamide.

2. The compound of formula (I) according to claim 1, or a tautomer, isomer, stereoisomer, a deuterated isotope, or a pharmaceutically acceptable salt and/or solvate thereof,
wherein, when not absent, -V-Z- is:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—CH₂—O—, —CH₂—CH₂—CH₂—, —CH₂—N(R18)- or —N(R18)-CH₂—; or
when -V-Z- is absent:
U is absent, CH₂ or —CH₂CH₂—.

3. The compound of formula (I) according to claim 1, or a tautomer, isomer, stereoisomer, a deuterated isotope, or a pharmaceutically acceptable salt and/or solvate thereof,
wherein, when not absent, -V-Z- is:
—CH₂—, —O—CH₂—, —O—C(CH₃)₂—, —CH₂—O—, —C(CH₃)₂—O—, —NH—CH₂—, —CH₂—NH—, —N(COCH₃)—CH₂, —CH₂—N(COCH₃), —CH₂—CH₂—, —O—CH₂—CH₂, —CH₂—O—CH₂, —CH₂—CH₂—O—, or —CH₂—CH₂—CH₂—; or,
wherein when -V-Z- is absent:
U is absent, CH₂ or —CH₂CH₂—.

4. The compound of formula (I) according to claim 2, or a tautomer, isomer, stereoisomer, a deuterated isotope, or a pharmaceutically acceptable salt and/or solvate thereof,
wherein -V-Z- is —CH₂—, —O—CH₂—, —CH₂—O—, —CH₂—CH₂—O—, —CH₂—N(R18)- or —N(R18)-CH₂— wherein R18 is:
(CH₂)₀₋₆-aryl, (CH₂)₀₋₆-heteroaryl^{a}, C(═O)R19, C(═O)OR19, C(═O)NHR19, C(═O)N(alkyl)(R19), or SO₂R19;
wherein R19 is alkyl, (CH₂)₀₋₆-aryl or (CH₂)₀₋₆-heteroaryl^{a}; or
when -V-Z- is absent:
U is absent, CH₂ or —CH₂CH₂—.

5. The compound of formula (I) according to claim 2, or a tautomer, isomer, stereoisomer, a deuterated isotope, or a pharmaceutically acceptable salt and/or solvate thereof,
wherein -V-Z- is —CH₂—, —O—CH₂—, —CH₂—O— or —CH₂—CH₂—O—.

6. The compound of formula (I) according to claim 1, or a tautomer, isomer, stereoisomer, a deuterated isotope, or a pharmaceutically acceptable salt and/or solvate thereof,
wherein X is a bond or CR1R2.

7. The compound of formula (I) according to claim 6, or a tautomer, isomer, stereoisomer, a deuterated isotope, or a pharmaceutically acceptable salt and/or solvate thereof,
wherein X is CH₂.

8. The compound of formula (I) according to claim 1, or a tautomer, isomer, stereoisomer, a deuterated isotope, or a pharmaceutically acceptable salt and/or solvate thereof,
wherein Y is O, CR1R2, N or NR12.

9. The compound of formula (I) according to claim 8, or a tautomer, isomer, stereoisomer, a deuterated isotope, or a pharmaceutically acceptable salt and/or solvate thereof,
wherein Y is N or NR12.

10. The compound of formula (I) according to claim 9, or a tautomer, isomer, stereoisomer, a deuterated isotope, or a pharmaceutically acceptable salt and/or solvate thereof,
wherein Y is N or NH.

11. The compound of formula (I) according to claim 1, or a tautomer, isomer, stereoisomer, a deuterated isotope, or a pharmaceutically acceptable salt and/or solvate thereof,
wherein Y is NR12.

12. The compound of formula (I) according to claim 11, or a tautomer, isomer, stereoisomer, a deuterated isotope, or a pharmaceutically acceptable salt and/or solvate thereof,
wherein Y is NH.

13. The compound of formula (I) according to any preceding-claim 1, or a tautomer, isomer, stereoisomer, a deuterated isotope, or a pharmaceutically acceptable salt and/or solvate thereof,
wherein B is heteroaryl^{a} or aryl.

14. The compound of formula (I) according to claim 13, or a tautomer, isomer, stereoisomer, a deuterated isotope, or a pharmaceutically acceptable salt and/or solvate thereof,
wherein B is heteroaryl^{a}.

15. The compound of formula (I) according to claim 14, or a tautomer, isomer, stereoisomer, a deuterated isotope, or a pharmaceutically acceptable salt and/or solvate thereof,
wherein B is a 9 or 10 membered bicyclic aromatic ring, containing 1 or 2 ring members that are, independently, N or NR12, optionally substituted as for heteroaryl^{a}.

16. The compound of formula (I) according to claim 15, or a tautomer, isomer, stereoisomer, a deuterated isotope, or a pharmaceutically acceptable salt and/or solvate thereof,
wherein B is:
isoquinolinyl, optionally substituted as for heteroaryl^{a}, or
azaindole, optionally substituted as for heteroaryl^{a}.

17. The compound of formula (I) according to claim 16, or a tautomer, isomer, stereoisomer, a deuterated isotope, or a pharmaceutically acceptable salt and/or solvate thereof,
wherein B is: 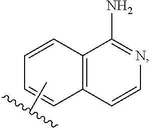
isoquinolinyl, substituted with NH₂ at the 1-position optionally further substituted with 1 or 2 substituents as for heteroaryl^{a}; or
7-azaindolyl 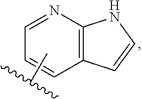
optionally substituted as for heteroaryl^{a}.

18. The compound of formula (I) according to claim 16, or a tautomer, isomer, stereoisomer, a deuterated isotope, or a pharmaceutically acceptable salt and/or solvate thereof,
wherein B is:
isoquinolinyl, that is 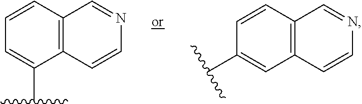 [SMILES: *c1cccc2cnccc12 ; *c1ccc2cnccc2c1]
optionally substituted as for heteroaryl^{a}; or
7-azaindolyl 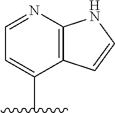 [SMILES: *c1ccnc2[nH]ccc12]
optionally substituted as for heteroaryl^{a}.

19. The compound of formula (I) according to claim 17, or a tautomer, isomer, stereoisomer, a deuterated isotope, or a pharmaceutically acceptable salt and/or solvate thereof,
wherein B is:
isoquinolinyl, substituted with NH₂ at the 1-position that is 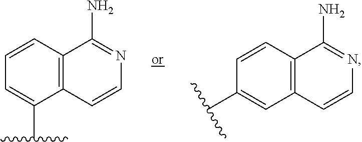 [SMILES: *c1cccc2c(N)nccc12 ; *c1ccc2c(N)nccc2c1]
optionally further substituted with 1 or 2 substituents as for heteroaryl^{a}; or
7-azaindolyl 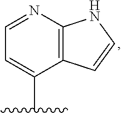 [SMILES: *c1ccnc2[nH]ccc12]
optionally substituted as for heteroaryl^{a}.

20. The compound of formula (I) according to claim 1, or a tautomer, isomer, stereoisomer, a deuterated isotope, or a pharmaceutically acceptable salt and/or solvate thereof, wherein AW- is —(CHR12)-A, —(CH₂)₀₋₆—C(═O)—(CH₂)₀₋₆-A, or —(CH₂)₀₋₆—O—(CH₂)₀₋₆-A.

21. The compound of formula (I) according to claim 20, or a tautomer, isomer, stereoisomer, a deuterated isotope, or a pharmaceutically acceptable salt and/or solvate thereof, wherein AW- is —(CHR12)-A, or —(CH₂)₀₋₆—O—(CH₂)₀₋₆-A.

22. The compound of formula (I) according to claim 1 or a tautomer, isomer, stereoisomer, a deuterated isotope, or a pharmaceutically acceptable salt and/or solvate thereof,
wherein A is a 4- to 12-membered mono- or bi-cyclic ring system, containing one N ring member and optionally one, two or three further ring members that are, independently, N, O or S, optionally wherein the ring system is substituted with 1, 2, 3 or 4 substituents that are, independently, halo, alkyl, OH, oxo, cycloalkyl, alkoxy, —(CH₂)₀₋₂-heteroaryl, heterocycloalkyl, C(═O)R12, C(═O)OR13, C(═O)NR13R14, NR13R14, CF₃, or CN;
wherein when A is a bicyclic ring system, the bicyclic ring system is fused, bridged or spiro.

23. The compound of formula (I) according to claim 22 or a tautomer, isomer, stereoisomer, a deuterated isotope, or a pharmaceutically acceptable salt and/or solvate thereof,
wherein A is:

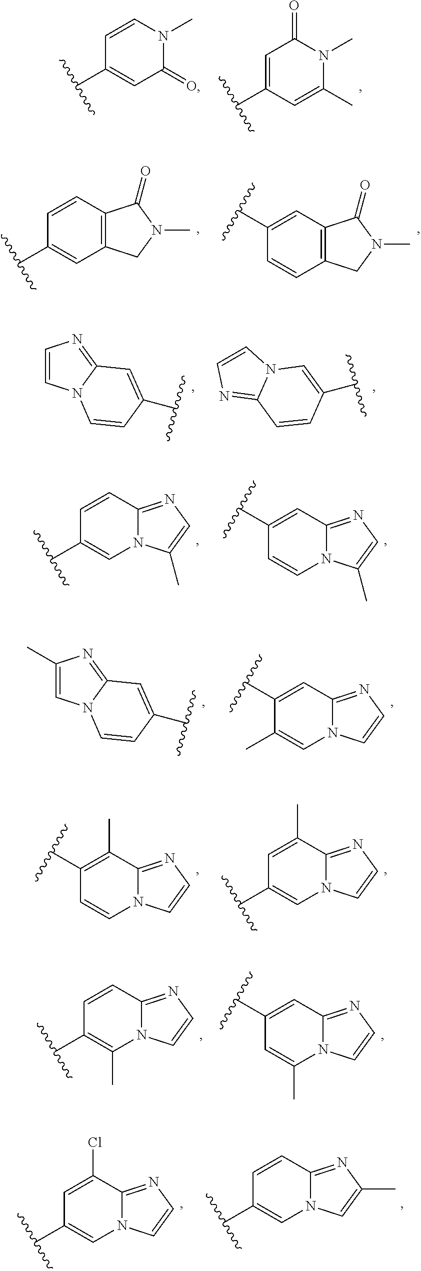

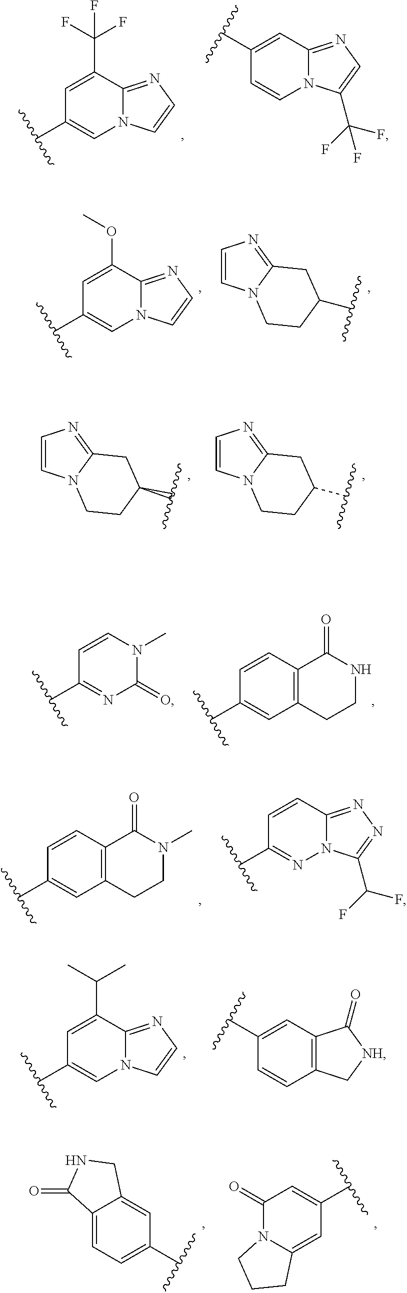

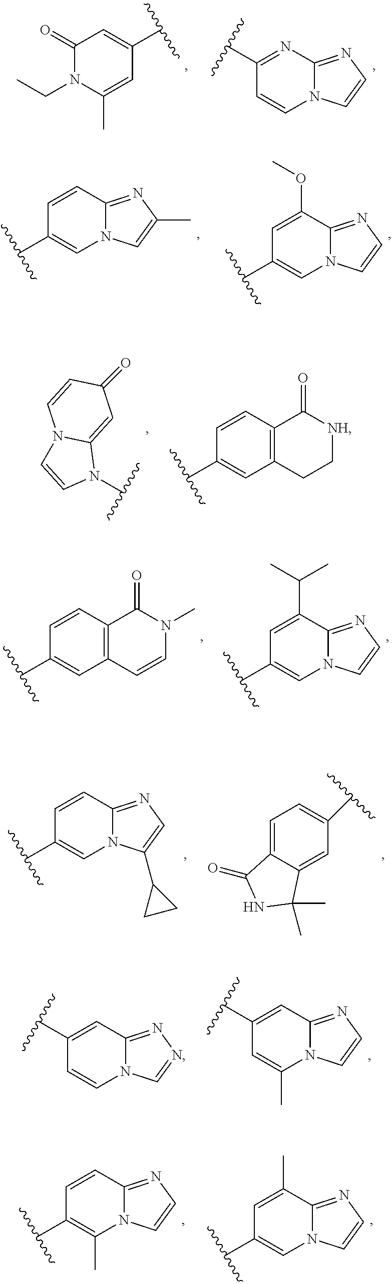

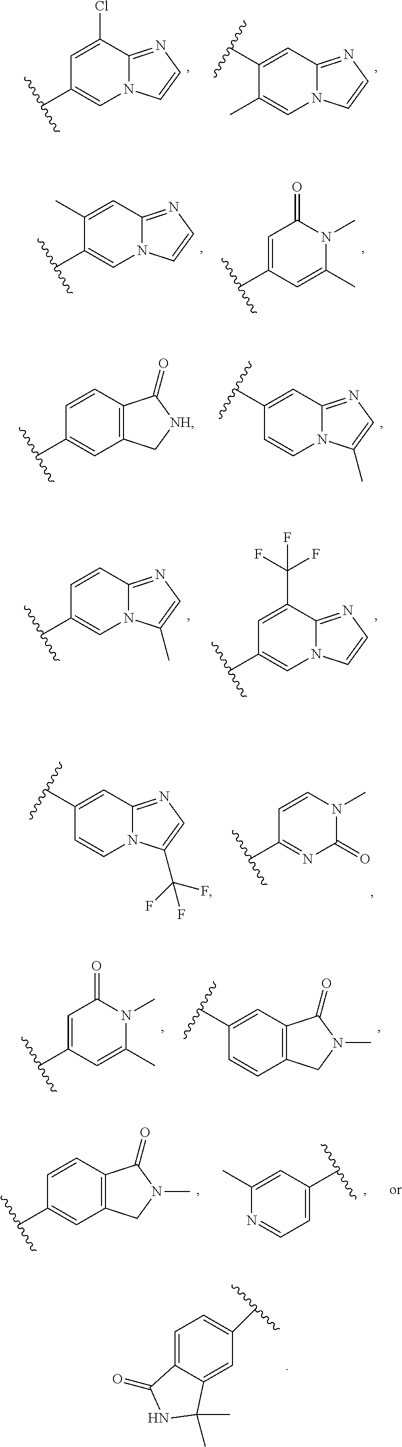

24. The compound of formula (I) according to claim 22 or a tautomer, isomer, stereoisomer,
a deuterated isotope, or a pharmaceutically acceptable salt and/or solvate thereof,
wherein A is:

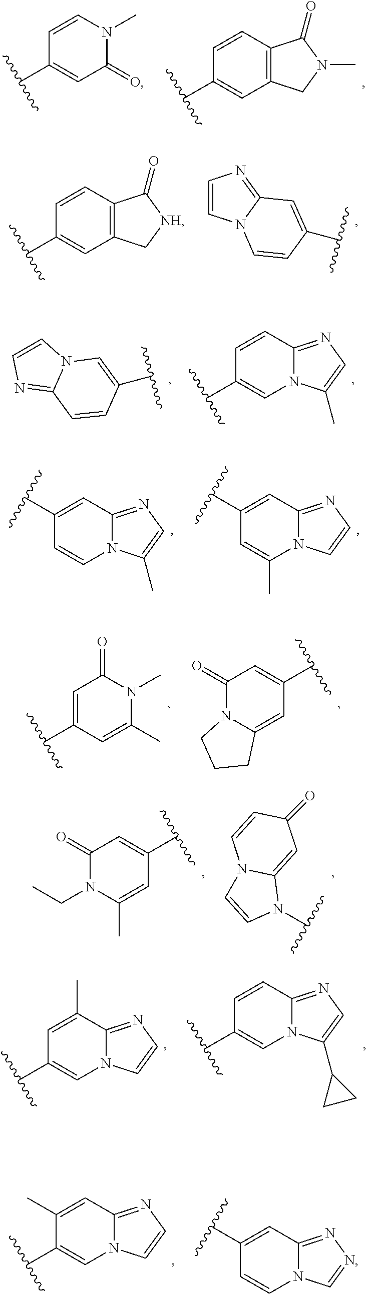

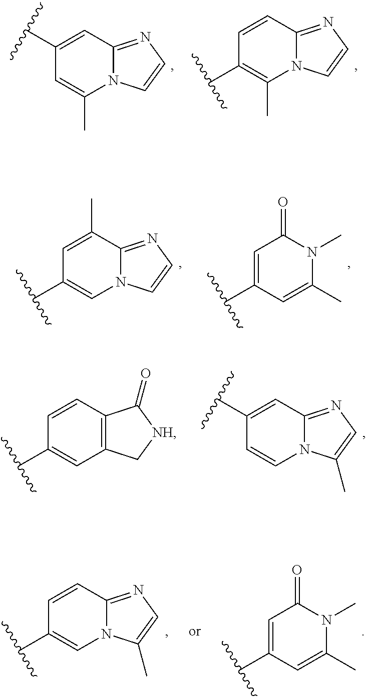

25. A compound that is from Table 1a, Table 1b, Table 2a, Table 2b, Table 3a, Table 4a, Table 5b, Table 6a, Table 6b, Table 7a, Table 7b, Table 8a, Table 8b, Table 9b, Table 10a, Table 10b, Table 11b, Table 12b, or Table 13b, or a pharmaceutically acceptable salt, solvate, or solvate of a salt thereof.

26. A pharmaceutical composition comprising: a compound, or a pharmaceutically acceptable salt and/or solvate thereof, according to claim 1, and at least one pharmaceutically acceptable excipient.

27. (canceled)

28. A method of treating a disease or condition in which Factor XIIa activity is implicated in a subject in need thereof, wherein the disease or condition in which Factor XIIa activity is implicated is a bradykinin-mediated angioedema, wherein the bradykinin-mediated angioedema is hereditary angioedema, the method comprising administering a compound of claim 1 to the subject.

29. A method of treating a disease or condition in which Factor XIIa activity is implicated in a subject in need thereof; wherein the disease or condition in which Factor XIIa activity is implicated is a bradykinin-mediated angioedema, wherein the bradykinin-mediated angioedema is non hereditary, the method comprising administering a compound of claim 1 to the subject.

30. A method of treating a disease or condition in which Factor XIIa activity is implicated in a subject in need thereof; wherein the disease or condition in which Factor XIIa activity is implicated is a thrombotic disorder, the method comprising administering a compound of claim 1 to the subject.

31. The compound according to claim 1, wherein the stereoisomer is an enantiomer, diastereoisomer, or racemic or scalemic mixture thereof.
